SEQUENCE LISTING (968 sequences)

SEQ ID NO: 1 - DNA, 31 bp, homo sapiens
gccttaggctactttcacagggcagaacaag

SEQ ID NO: 2 - DNA, 102 bp, homo sapiens
ctttcatgaattctgcatccgaggttggtgtatcgttgggaaaaagcagacttgcccttactgcaaagagaaagttgatttgaagaggatgatcagtaatcc

SEQ ID NO: 3 - DNA, 34 bp, homo sapiens
ggcggcggcgggaagatgatgctgctctttggcg

SEQ ID NO: 4 - DNA, 143 bp, homo sapiens
attccaaaccactacaacccttgccaaacacagcccactccatttcagaccgccttcctgaggagaaaatgcagactcagggttccagtaaccagtgatggattcaccccatctcccaaataaagtttacttgttttacattc

SEQ ID NO: 5 - DNA, 167 bp, homo sapiens
gctattaattctaatggctggattttgccctcttcttgtggagtataccatggtgaaaatatatcaaaacagttacacctgggaactttctcctcatttgtatttccttgctcctggacaacaaccacatgaccctctcactcaactactgatcatcaataaaacag

SEQ ID NO: 6 - DNA, 135 bp, homo sapiens
gttctggcccctgtcatcctcatgctgctcaattccttcttcaacgccatctccactcttgtggtcatcatggccagcacgccgctcttcactgtggtcatcctgcccctggctgtgctctacaccttagtgcag

SEQ ID NO: 7 - DNA, 713 bp, homo sapiens
gccatccagatcctgggcggcatgggctacgtgacagagatgccggcagagcggcactaccgcgacgcccgcatcactgagatctacgagggcaccagcgaaatccagcggctggtgatcgccgggcatctgctcaggagctaccggagctgagcccgcggcggactgccccaggactgcgggaaggcgcgggagccaggggcctccaccccaaccccggctcagagactgggcggcccggcgggggctccctggggaccccagatgggctcagtgctgccacccagatcagatcacatgggaatgaggccctccgaccattggcagctccgcctctgggcctttccgcctcctcaccactgtgcctcaagttcctcatctaagtggccctggcctcctgggggcggggttgtgggggggctgagcgacactcagggacacctcagttgtcctcccgcgggccctggtgccctggcatgaaggcccagtgcgacaggcccttggtggggtctgtcttttccttgaggtcagaggtcaggagcagggctggggtcaggatgacgaggcctggggtcctggtgttgggcaggtggtggggctgggccatggagctggcccagaggcccctcagccctttgtaaagtctgatgaaggcaggggtggtgattcatgctgtgtgactgactgtgggtaataaacacacctgtccccca

SEQ ID NO: 8 - DNA, 110 bp, homo sapiens
gtaaatgaatataaagaaaatcaaaacatcgcttatgtgtctctgagaccagcacagactacagttttaataaaaacagctaaggtctatcttgcccccttttcactcag

SEQ ID NO: 9 - DNA, 59 bp, homo sapiens
gccacctggaggatatgttgctcaccaccctttctgggcttcagcatgtactcccaccg

SEQ ID NO: 10 - DNA, 137 bp, homo sapiens
ggagggaggggtgggaagcaccatgcagtttgtatcctgggccacactgctaacgctcctggtgcgggacctggccgagatggggagcccagacgccgcggcggccgtgcgcaaggacaggctgcacccgaggcaag

SEQ ID NO: 11 - DNA, 163 bp, homo sapiens
ctgggcacattccggtctcatgatggggattattttattgaaccactacagtctatggatgaacaagaagatgaagaggaacaaaacaaaccccacatcatttataggcgcagcgccccccagagagagccctcaacaggaaggcatgcatgtgacacctcag

SEQ ID NO: 12 - DNA, 147 bp, homo sapiens
gtttaacatgcctcatgatgacaacaacaaatgtaaagaagaaggagttaagagtccccagcatgtcatggctccaacactgaacttctacaccaacccctggatgtggtcaaagtgtagtcgaaaatatatcactgagtttttaga

SEQ ID NO: 13 - DNA, 165 bp, homo sapiens
tgttcaaaaagctgtgacggtgggacccagaggagaagggctatttgtgtcaatacccgaaatgatgtactggatgacagcaaatgcacacatcaagagaaagttaccattcagaggtgcagtgagttcccttgtccacagtggaaatctggagactggtcagag

SEQ ID NO: 14 - DNA, 165 bp, homo sapiens
tgcttggtcacctgtggaaaagggcataagcaccgccaggtctggtgtcagtttggtgaagatcgattaaatgatagaatgtgtgaccctgagaccaagccaacatctatgcagacttgtcagcagccggaatgtgcatcctggcaggcgggtccctggggacag

SEQ ID NO: 15 - DNA, 270 bp, homo sapiens
tgctctgtgacctgtgggcaaggtagggcaacccggcaagtgatgtgtgtcaactacagtgaccacgtgatcgatcggagtgagtgtgaccaggattatatcccagaaactgaccaggactgttccatgtcaccatgccctcaaaggaccccagacagtggcttagctcagcaccccttccaaaatgaggactatcgtccccggagcgccagccccagccgcacccatgtgctcggtggaaaccagtggagaactggcccctggggagca

SEQ ID NO: 16 - DNA, 174 bp, homo sapiens
tgcactaagctgtgtggtggaggcataagaacaagactggtggtctgtcagcggtccaacggtgaacggtttccagatttgagctgtgaaattcttgataaacctcccgatcgtgagcagtgtaacacacatgcttgtccacacgacgctgcatggagtactggcccttggagc

SEQ ID NO: 17 - DNA, 174 bp, homo sapiens
tgctctgtgtcctgtggccgaggcgtacagcagaggcatgtgggctgtcagatcggaacacacaaaatagccagagagaccgagtgcaacccatacaccagaccggagtcggaacgcgactgccaaggcccacggtgtcccctctacacttggagggcagaggaatggcaagaa

SEQ ID NO: 18 - DNA, 95 bp, homo sapiens
gatggtacccgagtcgtagggaaatgcggtggttactgtggaaaatgcactccatcctctggtactggcctggaggtgcgagttttatagctaag

SEQ ID NO: 19 - DNA, 159 bp, homo sapiens
agaccaaagagattaggaaaagcctggcagtattctccaactccaaacaagctctagagtgctccaggaaaagttatattcagtatatgaataagtgttattctccattattaatgtgttctgaaaatatattatgaataaatacatcaccacacccaa

SEQ ID NO: 20 - DNA, 177 bp, homo sapiens
tgcactaagctgtgtggtggaggcataagaacaagactggtggtctgtcagcggtccaacggtgaacggtttccagatttgagctgtgaaattcttgataaacctcccgatcgtgagcagtgtaacacacatgcttgtccacacgacgctgcatggagtactggcccttggagctcg

SEQ ID NO: 21 - DNA, 143 bp, homo sapiens
cttacagtttccccagagtcagtattaaaggtggatgttgatatatgaaagttatgccttctcaccatgcttctgaaaacaacaggattttcactttgaatctatctgcaccgtttattagccagttctacaaggaatcattg

SEQ ID NO: 22 - DNA, 36 bp, homo sapiens
gaagctgccacttttgctagagagcaaggctttgag

SEQ ID NO: 23 - DNA, 103 bp, homo sapiens
cattaaagagatagccaaaaagacacaagccctgccaaagatgaactcaaagaggcagcgaatcgtgatcttcacccaagggagagatgacactataatggct

SEQ ID NO: 24 - DNA, 75 bp, homo sapiens
agaaaatattctctttggaatgggaaatcctctgcttgacatctctgctgtagtggacaaagatttccttgataa

SEQ ID NO: 25 - DNA, 171 bp, homo sapiens
ggcttttttcttacagtttccccagagtcagtattaaaggtggctcaccatgcttctgaaaacaacaggattttcactttgaatctatctgcaccgtttattagccagttctacaaggaatcattgatgaaagttatgccttatgttgatatactttttggaaatgagaca

SEQ ID NO: 26 - DNA, 115 bp, homo sapiens
actaaagacattaaagagatagccaaaaagacacaagccctgccaaagatgaactcaaagaggcagcgaatcgtgatcttcacccaagggagagatgacactataatggctacag

SEQ ID NO: 27 - DNA, 179 bp, homo sapiens
ctttccaggccgcctacatcggcatcgaggtgctcatcgccctggtctctgtgcccgggaacgtgctggtgatctgggcggtgaaggtgaaccaggcgctgcgggatgccaccttctgcttcatcgtgtcgctggcggtggctgatgtggccgtgggtgccctggtcatccccctcgcc

SEQ ID NO: 28 - DNA, 110 bp, homo sapiens
agtgtccccccattgggatggagtcacaccgtattgaggacaaccagatccgagcctcctccatgctgcgccacggcctgggggcacagcgcggccggctcaacatgcag

SEQ ID NO: 29 - DNA, 135 bp, homo sapiens
tggagaaaattccagtgtcagcattcttgctccttgtggccctctcctacactctggccagagataccacagtcaaacctggagccaaaaaggacacaaaggactctcgacccaaactgccccagaccctctcca

SEQ ID NO: 30 - DNA, 96 bp, homo sapiens
gtgttccaggctggtgatgtgccttcggaacgcccattctatcccccaaccttggtctccaacctgcccccagcctccccatgtgcccaggtggag

SEQ ID NO: 31 - DNA, 38 bp, homo sapiens
atgtcctgcggtttgacaacacctacagcttcattcat

SEQ ID NO: 32 - DNA, 148 bp, homo sapiens
cttcttgcgggagccaaagtgaacgaattaaccaagcatcgccagactgccctccatcttgctgcccagcaggacctgcccaccatctgctcagtcctcctagagaatggcgtggactttgctgccgtggatgagaatggaaacaatg

SEQ ID NO: 33 - DNA, 153 bp, homo sapiens
atgtatgagaactttgtggaggaggtggatgctgtggacaatgggatctcccagtgggcagagggggagcctcgatatgcactgaccactaccctgagtgcacgagttgctcgacttaatcctacctggaaccaccccgaccaagacactgag

SEQ ID NO: 34 - DNA, 39 bp, homo sapiens
tttgaagtgatggtagccgggaagttgattcactctaag

SEQ ID NO: 35 - DNA, 25 bp, homo sapiens
tggcgcttgaggctacaagtccaag

SEQ ID NO: 36 - DNA, 120 bp, homo sapiens
gctgcaagctgcaagctggtctctcttccacggtattcatggagcaccaactcagtgccaggcactgtcataggtcctggggtctagtcatggccctgctctctggagaccacataccag

SEQ ID NO: 37 - DNA, 26 bp, homo sapiens
gtaagaataaagtggatgaaaacatg

SEQ ID NO: 38 - DNA, 381 bp, homo sapiens
gtgactcctatagcagcagcccagacagcacacctatggggagcattgagtcactctcttctcattcctctgaacaaaatagcactacaaagtcagcttcctgccagcccagggagaaatctggagggattccttggattgcaaccccatcatcttccaatggacagaaaagccttggtctgtggacaactagtcctgaatcaagttccagagaagatgcaaccaagacagatgcagaatcagactgccagagtgttgcttcagtcactagcccaggagacgtttccccacccatagacctagtcaagaaagagccttatgggctttcaggactgaaaagagcttctgcttcttctctcagatccatctctgcagctgaag

SEQ ID NO: 39 - DNA, 157 bp, homo sapiens
gaaacaagagctacagtggatctattcaaagcttaacttctgtaggttccaaggagacacccaaagcttcaccaaacccagacctgcctccgaaaatgtgcaggagattaagactagacactgcctcaagcaatggctatcagcggcctggctcagt

SEQ ID NO: 40 - DNA, 66 bp, homo sapiens
agatgatgtgatccaaatctgtggccctgcagatggaatcagactttttaatgcattaaaaggccg

SEQ ID NO: 41 - DNA, 91 bp, homo sapiens
gggcagatttattgaaattaactagagatgatgtgatccaaatctgtggccctgcagatggaatcagactttttaatgcattaaaaggccg

SEQ ID NO: 42 - DNA, 25 bp, homo sapiens
tgggtccaagcctctcccgggtggc

SEQ ID NO: 43 - DNA, 28 bp, homo sapiens
tgggtccaagcctctcccgggtggccag

SEQ ID NO: 44 - DNA, 82 bp, homo sapiens
gtttgtaggtctgttggcctgtaggtttcggcacaagtttcagcgagagaaggagaaaactgccttggttggaaccttgcag

SEQ ID NO: 45 - DNA, 353 bp, homo sapiens
cctcagccccggaagccgagcgagcggccggcgcgctcatcgccggggagcccgccaggtggaccggcccgcgctccgcccccagcgagccggggacccacccaccaccccccgcaccgccgacgccgcctcgttcgtccggcccagcctgaccaatgccgcggtggaaacgggcttggagctggccccataagggctggcggcttcctccgacgccgcccctccccacagcttctcgactgcagtggggcggggggcaccaacacttggagatttttccggaggggagaggattttctaagggcacagagaatccattttctacacattaacttgagctgctggagggacac

SEQ ID NO: 46 - DNA, 82 bp, homo sapiens
gtgtggctatttacacgggaatggaaaccaagatggcattaaattatcaatcaaaatctcagaagcgatctgccgtggaaaa

SEQ ID NO: 47 - DNA, 198 bp, homo sapiens
ttcctcaaggcattcacggacttcctggccttcatggtcctctttaactacatcatccctgtgtccatgtacgtcacggtcgagatgcagaagttcctcggctcttacttcatcacctgggacgaagacatgtttgacgaggagactggcgaggggcctctggtgaacacgtcggacctcaatgaagagctgggacag

SEQ ID NO: 48 - DNA, 135 bp, homo sapiens
ctggagttcaagagctggaatcacgcgtcctggtgcctggagatttattaattttgacagggaacaaagtgctaatgccatgtgatgccgttctgattgaaggcagctgtgtggtggatgaaggcatgctgacag

SEQ ID NO: 49 - DNA, 87 bp, homo sapiens
gaaatggctttttctggggacgatttccacatcaagggagtgccggcacatgccatggtagttaagccctgcagaacagccagccag

SEQ ID NO: 50 - DNA, 218 bp, homo sapiens
cttcattgcccatgccattcgccactgctaccagcctgtgggaggaggagggagcccatcggacttctacctgtgctctctgctggccagcggcaccgcagccctggcctgtgtcttcctgggggtcaccgtggaccgatttggccgccggggcatccttcttctctccatgacccttaccggcattgcttccctggtcctgctgggcctgtgggatt

SEQ ID NO: 51 - DNA, 111 bp, homo sapiens
gtggttgtactacaatcccagagtcagacctagaagaaagatcagtagaacaagactctacagaactgtttaccaaccacagacatctcactgcagagacacccaggcctg

SEQ ID NO: 52 - DNA, 95 bp, homo sapiens
tagccctaagtcaaagcaggaggtgatggtccgtccccctacagtgatgtccccatctggaaacccccagctggattccaaattctccaatcagg

SEQ ID NO: 53 - DNA, 190 bp, homo sapiens
aatgtaattctgctgaccacataaagtcccaggattcccagcacacaccacactcgatgaccccatcaaatgctacagcccccaggtcttctaccccctcccatggccaaactactgccacagagcccacacctgctcagaagactccagccaaagtggtgtacgtgttttctactgagatggccaataa

SEQ ID NO: 54 - DNA, 317 bp, homo sapiens
tagccctaagtcaaagcaggaggtgatggtccgtccccctacagtgatgtccccatctggaaacccccagctggattccaaattctccaatcagggtaaacaggggggctcagccagccaatcccagccatccccctgtgactccaagagtgggggccatacccctaaagcactccctggcccaggtgggagcatggggctgaagaatggggctggaaatggtgccaagggcaaggggaaaagggagcgaagtatttccgccgactcctttgatcagagagatcctgggactccaaacgatgactctgacattaaag

SEQ ID NO: 55 - DNA, 144 bp, homo sapiens
aatgctgcggaacaaccgcatcagctgcatccacaacgacagcttcacgggcctgcgcaacgtccggctcctctcgctctacgacaaccagatcaccaccgtatccccaggagccttcgacaccctccagtccctctccacact

SEQ ID NO: 56 - DNA, 131 bp, homo sapiens
gtctccacggcagaggacaatgggatccttctgtacaacggggacaacgaccacattgcagttgagctgtaccagggccatgtgcgtgtcagctacgacccaggcagctaccccagctctgccatctacag

SEQ ID NO: 57 - DNA, 34 bp, homo sapiens
ccattcaccgcggagagggctgctctcgctgccg

SEQ ID NO: 58 - DNA, 112 bp, homo sapiens
actctgggacatctggagaaagctgtagtcttggaattaactttgaaacacttaaaagctttaaccgccttaaccgagcaacagcatcagaagataattgctttacagaatg

SEQ ID NO: 59 - DNA, 92 bp, homo sapiens
gaaagacccaagggagtggataacttggccctggaaccctgaccctgtgtctcctgcccggtggcagtaacaaagccttctgtctgcccaga

SEQ ID NO: 60 - DNA, 115 bp, homo sapiens
aaaaagaaaaaaccaaactgcagaagcagagagaggatgagctaatccagaagatccacaaactggtgcagaagagagacttcctggtggacgatgcggaggtcgagcggttaag

SEQ ID NO: 61 - DNA, 158 bp, homo sapiens
gcttttagagcactgtgatgtaacatgtcaagcagaaatagggagcatgtttacagccattctatgaaaaagtgttcggaatgtacagactagcacagaagctggactaattgaacaagtattgctgaaaatgagtgctgtagatgacatgatagcag

SEQ ID NO: 62 - DNA, 500 bp, homo sapiens
aactcaagctgatgcttcaaaaacgagaagcacctgttccaactaagactaaagtggccgttgatgagaataaagccaaagaattccttggcagcctgaagcgccagaagcggcagctgtgggaccggactcggcccgaggtgcagcagtggtaccagcagtttctctacatgggctttgacgaagcgaaatttgaagatgacatcacctattggcttaacagagatcgaaatggacatgaatactatggcgattactaccaacgtcactatgatgaagactctgcaattggtccccggagcccctacggctttaggcatggagccagcgtcaactacgatgactactaaccatgacttgccacacgctgtacaagaagcaaatagcgattctcttcatgtatctcctaatgccttacactacttggtttctgatttgctctatttcagcagatctttctacctactttggtgatcaaaaaagaagagttaaaacaacac

SEQ ID NO: 63 - DNA, 500 bp, homo sapiens; identical to SEQ ID NO: 62

SEQ ID NO: 64 - DNA, 389 bp, homo sapiens
aaatttgaagatgacatcacctattggcttaacagagatcgaaatggacatgaatactatggcgattactaccaacgtcactatgatgaagactctgcaattggtccccggagcccctacggctttaggcatggagccagcgtcaactacgatgactactaaccatgacttgccacacgctgtacaagaagcaaatagcgattctcttcatgtatctcctaatgccttacactacttggtttctgatttgctctatttcagcagatcttttctacctactttgtgtgatcaaaaaagaagagttaaaacaacacatgtaaatgccttttgatatttcatgggaatgcctctcatttaaaaatagaaataaagcattttgttaaaaagaa

SEQ ID NO: 65 - DNA, 83 bp, homo sapiens
gccagggacctggaagatctcagcccgattctcagatggcctggaatccaacagcagcacccagtttgaggtgaagaaatatg

SEQ ID NO: 66 - DNA, 83 bp, homo sapiens; identical to SEQ ID NO: 65

SEQ ID NO: 67 - DNA, 83 bp, homo sapiens; identical to SEQ ID NO: 65

SEQ ID NO: 68 - DNA, 83 bp, homo sapiens; identical to SEQ ID NO: 65

SEQ ID NO: 69 - DNA, 34 bp, homo sapiens
tggataaaaacctgactgtgagcgtccacgtgtc

SEQ ID NO: 70 - DNA, 123 bp, homo sapiens
tggatggtgtcccccatggtcgctgagactccccttcgaggattgcacccgcccgtcctaagcctccccacaaggcgaggggagttacccctaaaacaaagctattaaagggacagaatactt

SEQ ID NO: 71 - DNA, 136 bp, homo sapiens
cttttgaagagggagaacagcagattgctcagttacatgggtctggattttaattctttgccgtatcaagtacctacttcagatgaaggaggcgcaagacaaaatccaggacttgcctatggaaacccatattctg

SEQ ID NO: 72 - DNA, 1329 bp, homo sapiens
ttgttattgaagatgataggattgatgacgtgctgaaaaatatgaccgacaaggcacctcctggtgtctaactcccccaaagacaatgagttaagggagagaataagaacggcggtaacagttattggcaaaaagcatgaaaagagaaagcactttgaaatttattactagcttgctacccacgatgaaatcaacaacctgtatctggtatcaggccgggagacagatgaggcgagaggaggaggaggaggaggagaaggctctgggctcctctgcaaaaataaaaataaaaaaataaataaaattttaaaaataataaaaattcactatatacacatataaagaaataaaaagaagtctcagttgcagctatttgtcaaaattaatatccatttctttttatatacggtgaatattgcgcaattatagatctggattttgaaccacttaatgaagcggcaacaccaggtgttttgaggtgttggcattcttcgctgatttggctgttcccaatgtttacattatttaatcttgcaaaaatggttctgtgcacttggatgtgaaatgctgtccagttttattttttttatgttgttatccttggatgtacaaaaaattcagaaaatgatctctgtagatattctgttttattttggtcatctttagaagttatcaggaatgtgtttaaaacaagaagagaacttttctaaggaatgatacatagaaaagattttattttaaaatgagttgtaaagcttgtgtttctttgttgctgcaagctatctgcccaagttaatgcaaatggacacattttttatgtcagaaaaacacacacacacacacacacacacacacacacacacgaaaaacaaagaaaaaaatgcttgagctttttctaacttccccttgcagtctgttgtgtgagcagcctgtttatttctctaatattatgtcagtttattctctttaatggactgtaaaaaaatgtaatcacaagagtgccaaatatcttgaaatgccaaaaggcattttagtttcttttctctgtgctctgagtccacgtacaggaatgcttggagtgtcttttctgttatttatagggattctcttaaggcacaccagctgcctgttttgcatggtatttgcaaaaatgcctcttgcgtgaggaaatcttttaccattttttgtttgcaactttggacctcaagaggtttcccttcccttccccgttccctcttttcttaattcaatattctgtatgttgcaccttgaaccagcacacagggctatttctccaatgtacaataaaagaattgttcctgtgtctca

SEQ ID NO: 73 - DNA, 95 bp, homo sapiens
atggtaaagatcagcctgtggcttttacgtattgtgccaaaacatccatcctgagcctggagaagctctcactcaacattgccactgactgggag

SEQ ID NO: 74 - DNA, 37 bp, homo sapiens
ggaagttgaaaataccatctccgtggatcggccagtg

SEQ ID NO: 75 - DNA, 100 bp, homo sapiens
agcaacttgctgagcttcgtcaggaatttcttcgacaagaagaccagcttcaggactataggaagaacaatacttaccttgtgaagaggttagaatatga

SEQ ID NO: 76 - DNA, 38 bp, homo sapiens
gtgggtgtgccctgcacctgcctcttcccccattctca

SEQ ID NO: 77 - DNA, 34 bp, homo sapiens
gacccaggacttaattacccagtgcccagttgtg

SEQ ID NO: 78 - DNA, 171 bp, homo sapiens
aaaattatatcagaaatggagtctaagttagtagaacaacaacttgcagaagaaaacaagcttttaaaggagcaagaaaacatgaaagagctagtagtcaaccttctccgcatgactcaaatcaaaattgatgaaaaggaacaaaagtccaaggatttcctgaaagctcag

SEQ ID NO: 79 - DNA, 249 bp, homo sapiens
atgaatggctatcaaagaaggatcaaaaatgcaactgagaaaatgatggctcttgttgctgagctgtccatgaaacaagccctaaccattgaactccaaaaggaagtcagggagaaagaagacttcatcttcacttgcaattccaggatagaaaaaggtctgccactcaataaggaaattgagaaagaatggttgaaagtccttcgagatgaagaaatgcacgccttggccatcgctgaaaagtctcag

SEQ ID NO: 80 - DNA, 95 bp, homo sapiens
atcgatgggctagaggagaagctgtcccagtgtcggagagacctggaggccgtgaactccagactccacagccgggagctgagcccagaggccag

SEQ ID NO: 81 - DNA, 201 bp, homo sapiens
ctcactttcctcttcaggccaatggagcgacagggcccagattgtcttccaaaagaagctgatgccctccccgccccaggatgctgggcaagggctcatcgggagcaggggctcatccggagcaggggccagctttgtggggcaggagctccaggtgggtggcaggggcaggtgaacagagctattttccgaattaatata

SEQ ID NO: 82 - DNA, 1301 bp, homo sapiens
tttttttttcatcttaatgagtttctcataaagacacatcttggggcataccaaccttcatattcattcagtgtatccattggtgttaatattaacaatttcaactaaaaacaacaaacataatttttctaagtgtttcgagctgtgagatgtcaatttctacaatttagtttctagacatgctgtatatttaataaactttatgtaaactttaaaatattgcactgcatttatgaaaaaagctttctttgcctactgcagctatctaacattttatgaaactgacgcatgtccttcattattgaggctaaaagctcttgttcagattgcttgaggtttgaaaaagaggtgtgctagctttgcttagtttatttcttatttttggtatacatatacatatatataacattagaatgtgtgtactggaaatgctatgataggtattttgtgttaatccaaatgcaatgatagtttcttgtatgaatgtgcaagaggcctgtgaccgaatgctacggttgtgtggaggaaacttggagatgctgcaggttcgtgtcctggaattaggcaacatttgtcaggaaaccagtacaaaggaccaatgtgagatgcactctttttcaaacaggagatcaccactgccagaaagtgatagaagacaagaagaaaaaggaaagagtgggtttccacaaacctggactcatggaataacatggagtgattgtacattgcacatatctcccctctaaaacctgtagtacaacttctcccctcaagctgatattctgtgtctctcacctcaatgtccacaacagtcaatctcaaacaaggtagctttgaacatctcaaacagagtagctttgaaaatcaacattttggtaccagtgttttcattagaaataagtgtttttaactccccagatataatattgtaattgggaaacctgaatagttttaagagtccagtggagcctcagtgttttgaatagagatgactcattacacataatggtaggagttttctaccatatcatattgaagatatgaagaggtattttccagcttcccaagtttgagagttgggcatttacctcttacacttcagtcaacaaacaaactagtaattcctaatgcataattaatttggatgcaagataagattaagtttctggagttttgttttgacttctgaggtgctaaaaaataccatgtaaattctccctaccagctagaagggtagcaagacgttgtagagtgcttatcagagagtaccatttacttcaattgatcaatataaatatatc

SEQ ID NO: 83 - DNA, 224 bp, homo sapiens
ccacatccagttccaccaccaatgggaacagggccacgggcagctctgatgaccggtcggcaccctcagagggggatgagtgggaccgcatgatctcggacatcagcagcgacattgaggcgctgggctgcagcatggaccaggactcagcatgatggacagtggatgggggggcacccacaccttccgcgcagtcgtcataggccttcccagaaggagctgcc

SEQ ID NO: 84 - DNA, 160 bp, homo sapiens
gagtgggaactggtggtgctggggaagttgaagtggaacctggcagctgtcactcctcatgacttcattgagcacatcttgcgcaagctgccccagcagcgggagaagctgtctctgatccgcaagcatgctcagaccttcattgctctgtgtgccaccg

SEQ ID NO: 85 - DNA, 608 bp, homo sapiens
gttggtgatatgcagtacagctgctgcaaggaacttgtaatgcagcaactgagaagtcttcagtcatcctcctgcacagacaactttgtgtcacctgccaactagcccctctgcctccaccccggggctttcagagcatagtgtgaaacctccttgcttggactaccatgagttctttggcttgttatgaatcctgtaaaaagggaaggtggctctggaagagcaactgagaaaaagttcccaactgagcccttggaaaaaaaataaaggggagaggggaaaggcaggctgaggtcagtagaggtcagggtggtctgatagactatgaccctgtcttccctttttctcctctgggcctgaagccagggagtatgaatgaatgttcaaatggcagcttgttttaccttccttctccagcaagggttggcattggcccctgggagcgctggaatatgaaaccaagaggcaggcctggctcagggctgaagggctggggctagctctgaccagttcttgctgctctacctgccagagctggccaggtccttccaacttcccaggcttatcccaaacacatatgtggtgcttccagtcccaactaccctgcc

SEQ ID NO: 86 - DNA, 34 bp, homo sapiens
cttccagggctcaccagatcaacctctaaatatc

SEQ ID NO: 87 - DNA, 25 bp, homo sapiens
aaagctgtagaggaaccccttaatg

SEQ ID NO: 88 - DNA, 34 bp, homo sapiens
ctatacaacctcctaggaaagctgtagaggaacc

SEQ ID NO: 89 - DNA, 126 bp, homo sapiens
accatggcttgatcgtgagtggacccagcaaggaccccgatctggccagtgggcacggtccctacagcttcacccttggtcccaaccccacggtgcaacgggattggcgcctccagactctcaatg

SEQ ID NO: 90 - DNA, 242 bp, homo sapiens
ggtttggattttgaggccaaaaaccagcacaccctgtacgttgaagtgaccaacgaggccccttttgtgctgaagctcccaacctccacagccaccatagtggtccacgtggaggatgtgaatgaggcacctgtgtttgtcccaccctccaaagtcgttgaggtccaggagggcatccccactggggagcctgtgtgtgtctacactgcagaagaccctgacaaggagaatcaaaagatcag

SEQ ID NO: 91 - DNA, 146 bp, homo sapiens
ctaccgcatcctgagagacccagcagggtggctagccatggacccagacagtgggcaggtcacagctgtgggcaccctcgaccgtgaggatgagcagtttgtgaggaacaacatctatgaagtcatggtcttggccatggacaatg

SEQ ID NO: 92 - DNA, 207 bp, homo sapiens
gtgacacagtggtcttgtccctgaagaagttcctgaagcaggatacatatgacgtgcacctttctctgtctgaccatggcaacaaagagcagctgacggtgatcagggccactgtgtgcgactgccatggccatgtcgaaacctgccctggaccctggaagggaggtttcatcctccctgtgctgggggctgtcctggctctgctgt

SEQ ID NO: 93 - DNA, 139 bp, homo sapiens
gactatgacatcacccagctccaccgaggtctggaggccaggccggaggtggttctccgcaatgacgtggcaccaaccatcatcccgacacccatgtaccgtcctcggccagccaacccagatgaaatcggcaacttta

SEQ ID NO: 94 - DNA, 332 bp, homo sapiens
ctgctgtgactgacctacagtggactttctctctggaatggaaccttcttaggcctcctggtgcaacttaatttttttttttaatgctatcttcaaaacgttagagaaagttcttcaaaagtgcagcccagagctgctgggcccactggccgtcctgcatttctggtttccagaccccaatgcctcccattcggatggatctctgcgtttttatactgagtgtgcctaggttgccccttattttttattttccctgttgcgttgctatagatgaagggtgaggacaatcgtgtatatgtactagaacttttttattaaagaaacttttccca

SEQ ID NO: 95 - DNA, 34 bp, homo sapiens
gcaactttataattgagaacctgaaggcggctaa

SEQ ID NO: 96 - DNA, 147 bp, homo sapiens
gactatgacatcacccagctccaccgaggtctggaggccaggccggaggtggttctccgcaatgacgtggcaccaaccatcatcccgacacccatgtaccgtcctcggccagccaacccagatgaaatcggcaactttataattgag

SEQ ID NO: 97 - DNA, 847 bp, homo sapiens
aacctgaaggcggctaacacagaccccacagccccgccctacgacaccctcttggtgttcgactatgagggcagcggctccgacgccgcgtccctgagctccctcacctcctccgcctccgaccaagaccaagattacgattatctgaacgagtggggcagccgcttcaagaagctggcagacatgtacggtggcggggaggacgactaggcggcctgcctgcagggctggggaccaaacgtcaggccacagagcatctccaaggggtctcagttcccccttcagctgaggacttcggagcttgtcaggaagtggccgtagcaacttggcggagacaggctatgagtctgacgttagagtggtggcttccttagcctttcaggatggaggaatgtgggcagtttgacttcagcactgaaaacctctccacctgggccagggttgcctcagaggccaagtttccagaagcctcttacctgccgtaaaatgctcaaccctgtgtcctgggcctgggcctgctgtgactgacctacagtggactttctctctggaatggaaccttcttaggcctcctggtgcaacttaatttttttttttaatgctatcttcaaaacgttagagaaagttcttcaaaagtgcagcccagagctgctgggcccactggccgtcctgcatttctggtttccagaccccaatgcctcccattcggatggatctctgcgtttttatactgagtgtgcctaggttgccccttattttttattttccctgttgcgttgctatagatgaagggtgaggacaatcgtgtatatgtactagaacttttttattaaagaaacttttccca

SEQ ID NO: 98 - DNA, 31 bp, homo sapiens
cctgaggtgccccttcagcctgacaaacacg

SEQ ID NO: 99 - DNA, 266 bp, homo sapiens
tttcagagactcctccactctctgggaatgacacggactccctctcctgcgacagtggcagttcggcaactagcactccgtgtgtgtcccgcctggtcactggccaccacctgtgggccagcaagaatggccgccatgtcctgggcctgattgaggactatgaggccctgctcaaacagatcagccagggacagaggctccttgctgaaatggacattcaaacccaagaggctcccagctccacaagtcaagagctgggaacaaag

SEQ ID NO: 100 - DNA, 124 bp, homo sapiens
attggagaaatgaaggcagaggtcaccaaactacataaaaaattgtttgaacaagaaaagaagttgcaaaacaccatgaagcttttgcagctgagcaagcgccaggaaaaagtcatctttgatc

SEQ ID NO: 101 - DNA, 205 bp, homo sapiens
gctgttgttcctgaagctgaaagctcagaacgagagagagaagtttgctttctgctccatgaaaggctgtgagaggataaagattaaagctctgattccaaagaacgcaggcgtcagtgactgcacagccacagcttaccccaagttcaccgagagggctgtcgtagacgtgccgatgcccaagaagctctttggttctcagctg

SEQ ID NO: 102 - DNA, 120 bp, homo sapiens
agagctcagaaatatgctcagcaagtcttgcagaaggaatgtcgtcccaagtttgccaagacatcaatggcgctgttatttgagcacaggtatagcgtggacttactcccttttgtgcag

SEQ ID NO: 103 - DNA, 224 bp, homo sapiens
aaggcccccaaagacagtgaagctgagtccaagtacgatcctccttttgggttccggaagttctccagtaaagtccagaccctcttggaactcttgccagagcacgacctccctgaacacttgaaagccaagacctgtcggcgctgtgtggttattggaagcggaggaatactgcacggattagaactgggccacaccctgaaccagttcgatgttgtgataag

SEQ ID NO: 104 - DNA, 187 bp, homo sapiens
gttaaacagtgcaccagttgagggatattcagaacatgttggaaataaaactactataaggatgacttatccagagggcgcaccactgtctgaccttgaatattattccaatgacttatttgttgctgttttatttaagagtgttgatttcaactggcttcaagcaatggtaaaaaaggaaaccctg

SEQ ID NO: 105 - DNA, 159 bp, homo sapiens
ccattctgggtacgactcttcttttggaagcaggtggcagaaaaaatcccactgcagccaaaacatttcaggattttgaatccagttatcatcaaagagactgcctttgacatccttcagtactcagagcctcagtcaaggttctggggccgagataag

SEQ ID NO: 106 - DNA, 131 bp, homo sapiens
ctataaaaatgtgcccggcggcaaagctgcagaaggggcaccgctgagctgccgagaggaagctcgctctgcccggctgccctcttgtagtccgccggcgaggggcagttctcggtgaggaggaagagagc

SEQ ID NO: 107 - DNA, 24 bp, homo sapiens
gcacagcacccgcgcgggccctcc

SEQ ID NO: 108 - DNA, 125 bp, homo sapiens
cacaacagctccagctggcagcatcacttcccgccaatttatccaacttctgccaaggctctgaaatgccaacaacgtcgaggcctgcacttgatgtcaagggtggcacctcacctgcgaaggag

SEQ ID NO: 109 - DNA, 77 bp, homo sapiens
gatgccaaccaagagatgagctccgtggcctactccaaccttgcggtgaaagatcgcaaagcagtggccattctgca

SEQ ID NO: 110 - DNA, 69 bp, homo sapiens
gccagtggggctcccactagttcctcgggatctccaataggctctcctacaaccacccctcccactaaa

SEQ ID NO: 111 - DNA, 192 bp, homo sapiens
cccccatccttcaacctgcaccccgcccctcacttgctggctagtatgcacctgcagaaacttaatagccagtatcaggggatggctgctgccactccaggccaacccggggaggcaggacccctgcaaaactgggactttggggcccaggcgggaggggcagaatcactctctccttctgctggtgcccag

SEQ ID NO: 112 - DNA, 225 bp, homo sapiens
agccctgctatcatcgattcggacccagtggatgaggaagtgctgatgtcgctggtggtggaactggggttggaccgagccaatgagcttccggagctgtggctggggcagaatgagtttgacttcactgcggactttccatctagctgctaatgccaagtgtccctaaagatggaggaataaagccaccaattctgttgtaaataaaaataaagttacttacaa

SEQ ID NO: 113 - DNA, 90 bp, homo sapiens
gagtccctgcttcgcagagcaccggacttggagtcagagagacctggctgcgaatcccggctccgccactttctagcgctgtgaccttgg

SEQ ID NO: 114 - DNA, 121 bp, homo sapiens
ttttcaggtggcgaaactgactggtctggttagagcgcgtccaagccaagccgcagtgctgtttggatttttaaatttctccttctgagtgagaaaatagcaatcgagatggaaccatccg

SEQ ID NO: 115 - DNA, 34 bp, homo sapiens
tagcgctgtgaccttggttttcaggtggcgaaac

SEQ ID NO: 116 - DNA, 34 bp, homo sapiens
cgagatggaaccatccgcacaacagctccagctg

SEQ ID NO: 117 - DNA, 34 bp, homo sapiens
tagcgctgtgaccttggcacaacagctccagctg

SEQ ID NO: 118 - DNA, 163 bp, homo sapiens
ctataaaaatgtgcccggcggcaaagctgcagaaggggcaccgctgagctgccgagaggaagctcgctctgcccggctgccctcttgtagtccgccggcgaggggcagttctcggtgaggaggaagagagcagcggacggcacagcacccgcgcgggccctcc

SEQ ID NO: 119 - DNA, 594 bp, homo sapiens
gatgccaaccaagagatgagctccgtggcctactccaaccttgcggtgaaagatcgcaaagcagtggccattctgcactaccctggggtagcctcaaatggaaccaaggccagtggggctcccactagttcctcgggatctccaataggctctcctacaaccacccctcccactaaacccccatccttcaacctgcaccccgcccctcacttgctggctagtatgcacctgcagaaacttaatagccagtatcaggggatggctgctgccactccaggccaacccggggaggcaggacccctgcaaaactgggactttggggcccaggcgggaggggcagaatcactctctccttctgctggtgcccagagccctgctatcatcgattcggacccagtggatgaggaagtgctgatgtcgctggtggtggaactggggttggaccgagccaatgagcttccggagctgtggctggggcagaatgagtttgacttcactgcggactttccatctagctgctaatgccaagtgtccctaaagatggaggaataaagccaccaattctgttgtaaataaaaataaagttacttacaa

SEQ ID NO: 120 - DNA, 34 bp, homo sapiens
atcagtgtcgcccccaggcgtcctgttcagcatc

SEQ ID NO: 121 - DNA, 165 bp, homo sapiens
gcacattgcaagcaacccgtgccttgatggtggttggcatcctcctgggagtgatagcaatctttgtggccaccgttggcatgaagtgtatgaagtgcttggaagacgatgaggtgcagaagatgaggatggctgtcattgggggtgcgatatttcttcttgcag

SEQ ID NO: 122 - DNA, 85 bp, homo sapiens
gtctggctattttagttgccacagcatggtatggcaatagaatcgttcaagaattctatgaccctatgaccccagtcaatgccag

SEQ ID NO: 123 - DNA, 586 bp, homo sapiens
gtacgaatttggtcaggctctcttcactggctgggctgctgcttctctctgccttctgggaggtgccctactttgctgttcctgtccccgaaaaacaacctcttacccaacaccaaggccctatccaaaacctgcaccttccagcgggaaagactacgtgtgacacagaggcaaaaggagaaaatcatgttgaaacaaaccgaaaatggacattgagatactatcattaacattaggaccttagaattttgggtattgtaatctgaagtatggtattacaaaacaaacaaacaaacaaaaaacccatgtgttaaaatactcagtgctaaacatggcttaatcttattttatcttctttcctcaatataggagggaagatttttccatttgtattactgcttcccattgagtaatcatactcaactgggggaaggggtgctccttaaatatatatagatatgtatatatacatgtttttctattaaaaatagacagtaaaatactattctcattatgttgatactagcatacttaaaatatctctaaaataggtaaatgtatttaattccatattgatgaagatgtt

SEQ ID NO: 124 - DNA, 34 bp, homo sapiens
acacctgtcaatgccaggtatgaatttggcccag

SEQ ID NO: 125 - DNA, 34 bp, homo sapiens
aacaccgggaatccaacacagcgggcatcgacat

SEQ ID NO: 126 - DNA, 134 bp, homo sapiens
ctttctttgatcctaatgatgcatcatgccaggaaattctttttgatcccaaaacttcagtttcagaattatttgccattttgagacagtgggttcctcaggtccaacaaaacattgacattattggaaatgag

SEQ ID NO: 127 - DNA, 54 bp, homo sapiens
attgacagatatgactcttttacattatacctgcaaatctggagctcatggtat

SEQ ID NO: 128 - DNA, 119 bp, homo sapiens
atgtggatgccacttgcagtgattttaattttggaacagctttgcatattgcagcatacaacttgtgtgcaggtgctgtgaagtgcctcttggagcagggagcaaatcctgcatttagg

SEQ ID NO: 129 - DNA, 167 bp, homo sapiens
gcatggctccaaaattatggtctgtgagaaggacccagccctcaagaaccgctgccacattcgctacatgcacctgttctccaccatcaagtacaaggtctccataagtgtcagcaatgccctgggccacaatgccacagctatcacctttgacgagttcaccattg

SEQ ID NO: 130 - DNA, 283 bp, homo sapiens
aagagcggaggaccaggaaccagagagagagagagagaaaagagagaggagagacagagcgcttgggggcgaaaggagagagggagggaagggtgggtaaggaggagagagcggtctgctgcaaaccccaggaggagagcttggagcccaagccagaactcgagccctagccggagccgttcacagggaggcggctgccgggaccgtcagccctgcatgatgcatctccggctcttctgcatcctgctcgccgcggtctcaggagccgagggctggggctact

SEQ ID NO: 131 - DNA, 162 bp, homo sapiens
ggtaacccaggacccccaggtcccagcggttctccaggcaaggatgggcccccaggtcctgcgggtaacactggtgctcctggcagccctggagtgtctggaccaaaaggtgatgctggccaaccaggagagaagggatcgcctggtgcccagggcccacca

SEQ ID NO: 132 - DNA, 34 bp, homo sapiens
gcccaaaaggggaaccagggccacatggtattca

SEQ ID NO: 133 - DNA, 113 bp, homo sapiens
gggaatactggattgaccccaaccagggctgcgcgcgggactcgttcagggttttttgcaacttcacggcgggaggagagacctgcctctatcccgacaagaagtttgagatc

SEQ ID NO: 134 - DNA, 62 bp, homo sapiens
gccacatttgcaaccttggccatctgtccagaacctgctcccacctcaggcccaggccaacc

SEQ ID NO: 135 - DNA, 154 bp, homo sapiens
gtgcactgctgcaatgggctctgagctggagacggcgatggagaccctcatcaacgtgttccacgcccactcgggcaaagagggggacaagtacaagctgagcaagaaggagctgaaagagctgctgcagacggagctctctggcttcctggat

SEQ ID NO: 136 - DNA, 340 bp, homo sapiens
gcccagaaggatgtggatgctgtggacaaggtgatgaaggagctagacgagaatggagacggggaggtggacttccaggagtatgtggtgcttgtggctgctctcacagtggcctgtaacaatttcttctgggagaacagttgagcagacagccacattgggcagcgcccttcctctccaccctcccagacctgcctcttccccctgcttccacctcaccccacttatccctctccataaccccacccttgcccaccccacccccacccccaccaagggcgcaagagtagcggtccaagcctgcaactcatctttcattaaaggcttctctctcaccagc

SEQ ID NO: 137 - DNA, 127 bp, homo sapiens
cggaatgtggtggtcagatccatgcagccacatcaggacgaatattgtcccctggctatccagctccgtatgacaacaacctccactgcacctggattatagaggcagacccaggaaagaccattag

SEQ ID NO: 138 - DNA, 48 bp, homo sapiens
tgcaagaagaatctggacagtaccactgtggccgtgcatggtgaggag

SEQ ID NO: 139 - DNA, 265 bp, homo sapiens
gagggctccgacggcactgacggccatggcgcgttcgaacctcccgctggcgctgggcctggccctggtcgcattctgcctcctggcgctgccacgcgacgcccgggcccggccgcaggagcgcatggtcggagaactccgggacctgtcgcccgacgacccgcaggtgcagaaggcggcgcaggcggccgtggccagctacaacatgggcagcaacagcatctactacttccgagacacgcacatcatcaaggcgcagagccag

SEQ ID NO: 140 - DNA, 210 bp, homo sapiens
ctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttca

SEQ ID NO: 141 - DNA, 125 bp, homo sapiens
ctcttcgtcatctgaccagccgacaccaagaagcagagatggcccagaatgcagttcgccttcactatggactaccagttgtggttaagctcttacacccaccatcccactggcctctgataaag

SEQ ID NO: 142 - DNA, 61 bp, homo sapiens
actgctgatcttggacttgatattggtgcccagggagaaccccttggatatcgccaggatg

SEQ ID NO: 143 - DNA, 145 bp, homo sapiens
ctcatcatactggctagtggtggaccccaagctttagtaaatataatgaggacctatacttacgaaaaactactgtggaccacaagcagagtgctgaaggtgctatctgtctgctctagtaataagccggctattgtagaagctg

SEQ ID NO: 144 - DNA, 113 bp, homo sapiens
agaatcacgtcctggaactgcatgttcttgcgactcttgggacttcatcttaacttctcgctgccccagccatgttttcaaccatggcatccctcccccaattagttccctgt

SEQ ID NO: 145 - DNA, 984 bp, homo sapiens
gtctgtaactggttcatcaacgcccgccgcaggctcctccctgacatgctgagaaaggatggcaaagatccaaatcagttcacaatttcccgccgtggggccaagatttctgaaacgagctctgtggagtccgtgatgggcatcaaaaacttcatgccagctctagaggagaccccatttcattcctgtacagctgggccaaacccaaccctagggaggccactgtctcctaagccgtcatccccgggatcagttttggctcgtccatcagtgatctgccataccactgtgactgcattgaaagatgtccctttctctctctgccagtcggtcggtgtgggacaaaacacagatatacagcagatagcggccaaaaacttcacagacacctctctcatgtacccagaggacacttgtaaatctggaccaagtacgaatacacagagtggtcttttcaacactcctccccctactccaccggacctcaaccaggacttcagtggatttcagcttctagtggatgttgcactcaaacgggctgcagagatggagcttcaggcaaaacttacagcttaacccattttcaagcaaaacagttctcagaaatgtcatgattgccggggtgaaggcaagagatgaattgcattattttatatattttttattaatatttgcacatgggattgctaaaacagcttcctgttactgagatgtcttcaatggaatacagtcattccaagaactataaacttaaagctactgtagaaacaaagggttttcttttttaaatgtttcttggtagattattcataatgtgagatggttcccaatatcatgtgatttttttttcctccccttccctttttttgttattttttcagactgtgcaatacttagagaacctatagcatcttctcattcccatgtggaacaggatgcccacatactgtctaattaataaattttccattttttttcaaacaa

SEQ ID NO: 146 - DNA, 34 bp, homo sapiens
atctccgttgattccagaatcgtccgcactaaag

SEQ ID NO: 147 - DNA, 145 bp, homo sapiens
gtgagttcatcaaagcgctgtatgagtcagatgagaactgcgaagtggatcccagcaagtgctcggccgctgacctcccagagcaccagggcaacctcaagatgtgctgcgagctggccttctgcaagatcatcaactcctactg

SEQ ID NO: 148 - DNA, 237 bp, homo sapiens
tgtcttcccacgggagttgaaagaggtgtttgcctcgtggaggcaggagtgcagcagtcgcggccgcccggacatcagtgagcggctcatcagcgcctccctcttcctgcgcttcctctgcccagccatcatgtcgccctcactcttcaacctgctgcaggagtaccctgatgaccgcactgcccgcaccctcaccctcatcgccaaggtcacccagaacctggccaactttgccaa

SEQ ID NO: 149 - DNA, 71 bp, homo sapiens
gagaagcgcattgcctcgttggatgccgccaatgcccgcctcatgagtgccctgacccagctgaaagagag

SEQ ID NO: 150 - DNA, 45 bp, homo sapiens
tgaaagctggtggaatgcgaattgtgcagaaacacccacatacag

SEQ ID NO: 151 - DNA, 52 bp, homo sapiens
gagacaccaaagaagagaaagacaaggatgaccaggaatgggaaagccccag

SEQ ID NO: 152 - DNA, 53 bp, homo sapiens
gtggactggttttcactggcgagcgtcatcttcctactgctgttcgccccctt

SEQ ID NO: 153 - DNA, 230 bp, homo sapiens
gccttcctgccacgtatgctggagctgcagaatggccacatcgtgtgcctcaactccgtgctggcactgtctgccatccccggtgccatcgactactgcacatccaaagcgtcagccttcgccttcatggagagcctgaccctggggctgctggactgtccgggagtcagcgccaccacagtgctgcccttccacaccagcaccgagatgttccagggcatgagagtcag

SEQ ID NO: 154 - DNA, 239 bp, homo sapiens
accaccaaggccttcctgccacgtatgctggagctgcagaatggccacatcgtgtgcctcaactccgtgctggcactgtctgccatccccggtgccatcgactactgcacatccaaagcgtcagccttcgccttcatggagagcctgaccctggggctgctggactgtccgggagtcagcgccaccacagtgctgcccttccacaccagcaccgagatgttccagggcatgagagtcag

SEQ ID NO: 155 - DNA, 109 bp, homo sapiens
tctagataaacttttaggggctataacaacagcagcagaaagaaataacagggaacgattttcaccaattgtggaaggtttagaaaatcaggaagccttgcaattacag

SEQ ID NO: 156 - DNA, 93 bp, homo sapiens
gtgaggaagatcttctcagtcctccccaggacacaagcacagggttagaggaggtgatggagcaactcaacaactccttccctagttcaagag

SEQ ID NO: 157 - DNA, 138 bp, homo sapiens
cacaaaagctacatcgaatacacaaagactctgccactgaccccagcaccagaaatctttgggatgaatgccaatgcagatatcactaaggatcagtcagaaactcagctgctatttgataacattcttcttacacag

SEQ ID NO: 158 - DNA, 123 bp, homo sapiens
gacattgtttaaggatgcatctggaaatcttgacaaaaatgccagattttctgccatctacaggcaagacagcaataagctatccaatgatgacatgctcaagttacttgcagactttcggaa

SEQ ID NO: 159 - DNA, 34 bp, homo sapiens
atgtccatttcaacgaggatgtgctgatggagct

SEQ ID NO: 160 - DNA, 101 bp, homo sapiens
gtcaatgctggcccactagcatatgcgcgagctttcttagatgatacaaacacaaagcgatatcctgacaataaagtgaagctgcttaaggaagttttcag

SEQ ID NO: 161 - DNA, 34 bp, homo sapiens
aacgccatcagtgggactccaacaagcacaatgg

SEQ ID NO: 162 - DNA, 134 bp, homo sapiens
agttcggacctctgggatcggcgattcccctctggccagggctggttttaatgctctccgtgggcgggcttcggttgagtttggtccgcttttcctttctgctcctcaggggagcattgcttccttctctcgca

SEQ ID NO: 163 - DNA, 121 bp, homo sapiens
ctggaagctgaggctgtgttccgtgccatcaccattgccagccaaaccaattgccctctctacgtcacaaaggtcatgagcaagagtgcagctgacctcatctcacaagccaggaaaaaag

SEQ ID NO: 164 - DNA, 90 bp, homo sapiens
aggatgagatcatagtttcaggatcctcaggaaaccctggtactggcagcagccagcctctgctgtgcccacatgacccacaactctggc

SEQ ID NO: 165 - DNA, 162 bp, homo sapiens
gaatcactgggatcctcatgagtgcagcggggctgcctgtgtgtctcaccaggccaccaaagctggtcctacacccaccccccgtcagcaagagtgaaataaaatccatcccaggggtttccaacacaagccgcaagaccaccaaaaaacaagccaagaaag

SEQ ID NO: 166 - DNA, 226 bp, homo sapiens
gtgctggacatcaaccaggccgacgcagggaccctgcccctggactcctcccagaaggtgcgggaggccctgacctgtgagctgagcagggccgagtttgccgagtccctgggcctcaagccccaggacatgtttgtggagtccatgttctctctggctgacaaggatggcaatggctacctgtccttccgagagttcctggacatcctggtggtcttcatgaaag

SEQ ID NO: 167 - DNA, 3039 bp, homo sapiens
aaaaaaagaaaagaaaaaaaaagacagcatctcactatgttgccaaggctcatctcaagctcttgggctcaagagatcctcccacctcggcctcctgagtagctgggactgcaggtgtgtgtcatcatgaccaatgtgaattgcttttgaagctggttcatgggcatgtaggccaccgaagcaattttagaccacagtaagtcaagcttttttccctccgatgatcactgggtggttgcagcattttttgcataaacctgcctaagacttgtctatcgtctgtgatcaatatgccatattacactaaggtgctcctggaaaattgggtgcagttcaaattttcctacagcaaatcatttggcaaggccagccattggggaaaccagacaactagagataaccctgaaatgaatccttttgtaaattgaagcaccatcttttctttttttgcataaattggaggttttaattttagggcagttacctgaagtgaaatataccaacaatttcttgtgttctttaaattcctagttaggtgaatatttttgaaggtcctcttttgaataaagaggggaatggacaccacatttcaggtcttctcgaagtgtggaagggcaagagagcatcagtgagctgatggtggattgcttacatcggattccattggtatgaatttcccaaactggaaatcaaagcgccagggtggggttggggctgactgctggtgagggggctggccgctggctcccgtgacgtgcgtcatgggcacgcaggcgccattttgaatctatcgtcggcacgtgggtgccattttgaatccttagttgggcctttctaaatggagaatggctttggagggagacacgttttctgtggggagggtttgggggggagggaggagggaacaagctacatgctattttgtttgtagtattgtggaacagtcttgttatggagtgccagcttagaggttgttgcaaacttgtctagaagtgagagcatggttttttttagccctttgagagtctacatctaatgaacattcttgctcacccataaataacgtcaagcctcaatgtcaccgtcacgttgggatactctttctcatctggcatcctagacaggacaaggttggttacctttccttccatgaaccatgaacctgtgacggcatcattcatcctgacttcaccaagctccgcctgtgggtgaggccagagctcccactggcaatttttagaagagccagaggctccctgcttcctctagaaataacagttcagggtgaagcatggagggtttcagttcccagacaatggaaccatttagagacaacacagttggacatttccactttttccttgattcctggaagtccagtgggttctgcagctgaaaaagccctgggtcccagcagcagagagacaggacagaggggatgcttgggcggggagggacggtaacctgcagaacagattccatttttatagaacgagtacacgtttgctaaaacagtcctgctttcccagactggattcccaccacagggacagtcggaactcaggactagctccagcgacatctttcctccgaattcaagccttctatcacaatgtcaaaacagctatttataaagccattttcattgtacttgataacagcacgagtcccaaaacttttagaaataaaataggacattggcttgattgaaaagagggactttttaaaaattgttctttcgtcagaagccttttggatgacttacaatagctctgatgaagataccaccccagcgtcagtccaataggtcagtgagtttcaacaggcatccatccctcccatgaagggattctggtgatgggaagtttctgtaatgacaggaaagcattgaccctcattgattgtcaactttggtattagccatgaaagacaggatgctcattgggtgttctgtagagtgaggaatgctgcctattccctcccagaacgtctgacccaggggtgtgtgttgaggagccctgggggaaatggaccaagttttcccacagagcagtattaggctgaagagcaggtgactggtaggccccagctcccatcattccctcccaaagccattttgttcagttgctcatccacgctggattccagagagttttccaatttgggaagccatgagaaaggtttttaaatcttgggaagatggagagagggacataggatagttgactccaacatgacaggaagaggctggagattgggaattggccatcaaccaagcctgtagtagtaaagccatggtcccgcattggaattacttggggaacttatacagttctgatacccaggctctcctagaccagttcaaccaattctaggtgggggactcaggcatcagtgtgtttcgtagctccccgggtgttttccctgtgcagccgagcttgggaaactgccatgctttttggatgtcaaggcgctgttggaggctgggtgtgacagcacagagccaggttgtcttgtggaaaccacagccacgggtttgccactggctcagcatggcctcactgccagtcccagcctggctgagggacaagatggtttctcttgggagttcctgagtggagcacccttccaggctttttgaaagccagctgatctgtggagccttgttaagggactcaatacggtgtttggatattgatgtttttccttgagactgtcttgtccatcaataaagatggaggatgtctcctctttgaaccccgcttccccaccagtactctctctcccttagagtttatgagttattcaaggaggagacttcttaaagacagcaacgcaattcttgtaacttgtgtaaatagccccatctttcagagtgataccatttctacatttgataatgcctgtattcctgtaggatgtatatagtttaggggattttttttttgtttggttttgttttttagaagtcaatatgtctggttttatttattgcttgaaaaagatcatttgaaaaaaataaatacattttcaaccact

SEQ ID NO: 168 - DNA, 63 bp, homo sapiens
ccccccgtctcccaggagctcccctgctccagcgagggcagccgtgagaacctgctgcaccag

SEQ ID NO: 169 - DNA, 34 bp, homo sapiens
accccaacggcctgcaggtgaacttccacagccc

SEQ ID NO: 170 - DNA, 213 bp, homo sapiens
gatccccctctgtgtgcctgagcgaagcttgtgtctcagtgaccagctccatcttgagctccatggaccccacagtggacccctgccatgacttcttcagctacgcctgtgggggctggatcaaggccaacccagtccctgatggccactcacgctgggggaccttcagcaacctctgggaacacaaccaagcaatcatcaagcacctcctcg

SEQ ID NO: 171 - DNA, 147 bp, homo sapiens
ctcgggggctggaacatcacaggtccctgggccaaggacaacttccaggacaccctgcaggtggtcaccgcccactaccgcacctcacccttcttctctgtctatgtcagtgccgattccaagaactccaacagcaacgtgatccag

SEQ ID NO: 172 - DNA, 107 bp, homo sapiens
acaactacatgatctggaacctggtgcggaaaacaagctccttccttgaccagcgctttcaggacgccgatgagaagttcatggaagtcatgtacgggaccaagaag

SEQ ID NO: 173 - DNA, 111 bp, homo sapiens
gtggagcatgaccccgcccatggtgaacgcctactactcgcccaccaagaatgagattgtgtttccggccgggatcctgcaggcaccattctacacacgctcctcacccaa

SEQ ID NO: 174 - DNA, 191 bp, homo sapiens
gacgggagtatgacaaggacgggaacctccggccatggtggaagaactcatccgtggaggccttcaagcgtcagaccgagtgcatggtagagcagtacagcaactacagcgtgaacggggagccggtgaacgggcggcacaccctgggggagaacatcgccgacaacgggggtctcaaggcggcctatcgg

SEQ ID NO: 175 - DNA, 221 bp, homo sapiens
tgggaggatacccttgacaaatactgtgaccgggagtatgctgtgaagacccaccaccacttgtgttgccgccaccctcccagccctactcgggatgagtgctttgcccgtcgggctccttaccccaactatgaccgggacatcttgaccattgacatcggtcgagtcacccccaacctcatgggccacctctgtggaaaccaaagagttctcaccaagca

SEQ ID NO: 176 - DNA, 36 bp, homo sapiens
acagcccagattattgtcaataatgaacagcctcag

SEQ ID NO: 177 - DNA, 60 bp, homo sapiens
agtcttttcctgacatctctctgaacaacgagaaggtgctggtggagaccagttacccca

SEQ ID NO: 178 - DNA, 66 bp, homo sapiens
gccaaaccactcgactgccccccatcacctatactggccgcttttccctggagcctgcacccaaca

SEQ ID NO: 179 - DNA, 70 bp, homo sapiens
gcctccgcctcccagagcccacccctgagctgcgcagtgccatccaacgacagcagtcccatttactcag

SEQ ID NO: 180 - DNA, 38 bp, homo sapiens
cggcacccaccttccccacgccgaacactgacattttc

SEQ ID NO: 181 - DNA, 243 bp, homo sapiens
cctgagccacaaagccaggccttcccgggctcggcagggacagcgctccagtacccgcctcctgcctaccctgccgccaagggtggcttccaggttcccatgatccccgactacctgtttccacagcagcagggggatctgggcctgggcaccccagaccagaagcccttccagggcctggagagccgcacccagcagccttcgctaacccctctgtctactattaaggcctttgccactcag

SEQ ID NO: 182 - DNA, 568 bp, homo sapiens
tcgggctcccaggacctgaaggccctcaataccagctaccagtcccagctcatcaaacccagccgcatgcgcaagtaccccaaccggcccagcaagacgcccccccacgaacgcccttacgcttgcccagtggagtcctgtgatcgccgcttctcccgctccgacgagctcacccgccacatccgcatccacacaggccagaagcccttccagtgccgcatctgcatgcgcaacttcagccgcagcgaccacctcaccacccacatccgcacccacacaggcgaaaagcccttcgcctgcgacatctgtggaagaaagtttgccaggagcgatgaacgcaagaggcataccaagatccacttgcggcagaaggacaagaaagcagacaaaagtgttgtggcctcttcggccacctcctctctctcttcctacccgtccccggttgctacctcttacccgtccccggttactacctcttatccatccccggccaccacctcatacccatcccctgtgcccacctccttctcctctcccggctcctcgacctacccatcccctgtgcaca

SEQ ID NO: 183 - DNA, 728 bp, homo sapiens
aaaaaaaaaagaaaagccaagcaaaccaatggtgatcctctattttgtgatgatgctgtgacaataagtttgaacctttttttttgaaacagcagtcccagtattctcagagcatgtgtcagagtgttgttccgttaacctttttgtaaatactgcttgaccgtactctcacatgtggcaaaatatggtttggtttttctttttttttttttttgaaagtgttttttcttcgtccttttggtttaaaaagtttcacgtcttggtgccttttgtgtgatgcgccttgctgatggcttgacatgtgcaattgtgagggacatgctcacctctagccttaaggggggcagggagtgatgatttgggggaggctttgggagcaaaataaggaagagggctgagctgagcttcggttctccagaatgtaagaaaacaaaatctaaaacaaaatctgaactctcaaaagtctatttttttaactgaaaatgtaaatttataaatatattcaggagttggaatgttgtagttacctactgagtaggcggcgatttttgtatgttatgaacatgcagttcattattttgtggttctattttactttgtacttgtgtttgcttaaacaaagtgactgtttggcttataaacacattgaatgcgctttattgcccatgggatatgtggtgtatatccttccaaaaaattaaaacgaaaataaagtagctgcgattggg

SEQ ID NO: 184 - DNA, 64 bp, homo sapiens
taagaaaatggccaaagccctctgccacctcaacgtccctgtcactgtggtgctagatgctgct

SEQ ID NO: 185 - DNA, 124 bp, homo sapiens
tgaacagagtatctgtcaggcaagagctgctgtgatggtttatgatgatgccaataagaagtgggtgccagctggtggctcaactggattcagcagagttcatatctatcaccatacaggcaac

SEQ ID NO: 186 - DNA, 727 bp, homo sapiens
caatcaggcaggaactgagcaagtcaaatactgcatagaggaacagactaaggagagataggactttaatctggaggaaaaatatcctacaaacaacaactgttcacaacagcaaacccctacatttatgagctgtaagaagaaaatggagacaaacagaaggagggaaaaaccaacctactctgaaagccttcagacattatgactctggtgataagctctttccctctccgtttgctgcttttttctggcctttacaacagaatggaagagaatcatttaagagttcctgtaacagttatgcagaaaatactaaaacccatcaggcaagatcaccacgcattgaaatattttcatatcaagataaagtcgcacattttccacaatacattgctaaaataaagaggagaaaggcttaggaagtttttttgcagagagtgctggtaaagaattgagcaagtttgctattgtattgtaatgtttctctcaggtttgttcttcctatcatgtttgatattccatgaataattgagatcagccctatgtaagttaagatcataatatgtggaacaaatggaattgtaagtgctttcaaagggtaatatttataagaaagtgtccgaaaaatgtttcttcagcttgagaaattttagaatgataggaagtttctcgagttagccttcatgcaattttgtagattaaaacataaaatttgtccagaacttaa

SEQ ID NO: 187 - DNA, 34 bp, homo sapiens
tatgattcattacacagaccaaaatccacaccct

SEQ ID NO: 188 - DNA, 34 bp, homo sapiens
tcacctgttatctccagaccaaaatccacaccct

SEQ ID NO: 189 - DNA, 1652 bp, homo sapiens
atcgatgaccccaacagcaaccttgaggaggcgattaatgaggcagaggccatcacctctgtgaacagcctgggaagcaagcaagccttgaatacagattaccttgattctgattaccaaagaggacagctttacccattctcccttagcagtgatgtccaggtggccacatttactctcacaaattcagccccaatgactcagtccttccaggaacggtggtatgtgaatctccacagcctaatggaccgggctttgaccccacagtgtggcagtggggaagacctatatatcctcacaggcacagtgccctcagactacagagttaaagacaaagtggcagtccctgagtttgtttggctggcagcctgttgtgctgtccctggaggaggctgggccatgggctttgtcaagcacacccgggacagtgacatcatagaagatgtgatggtaaaagatcttcagaaactgcttccatttaaccctcagctgtttcagaacaactgtggtgaaactgagcaagacacagagaaaatgaaaaaaatcctggaagtggttaaccaaatccaggatgaagaacgaatggtacaatctcaaaagagttctagtcccctttctagcaccaggagcaagaggtctactctgttgcctccagaggcatctgagggaagtagtagctttttgggaaaactcatgggcttcattgctaccccattcatcaagctttttcaattaatttattaccttgtggtagcaatcctgaagaacattgtctatttcctgtggtgtgttaccaagcaggtgattaatggcatagaaagttgcctttaccgcctgggctcagccaccatctcatacttcatggccattggggaagagttggtgagcattccctggaaggtgctcaaggtcgtggccaaagtcatcagggctctcctccggatcctttgttgtctgctgaaggccatttgccgagttctgagcatccctgtccgtgtccttgtggatgtggccactttccctgtgtacaccatgggcgctattccaattgtttgcaaggacattgcactgggccttggtggcactgtctcactgctctttgacactgcttttggtaccctgggtggcctatttcaggtggtttttagtgtctgcaagcggattggctacaaggttacttttgacaattctggggagttataaactcaaaaaactaatagtatccagtcacagtgaatttgaaagctggaatagtttgtctttacaatgggtttctgttcactgtcagttatcattatattttggcctttggtggggatgtctgcttgtttttgcaaaagaagatggcagaatttagacttgacagaggagaaatgctcagggtgagattaggtgtagtaatctgctgtttacctccagttatatgtgcaaactcccaagccactaataacttcagttatgcactctaacacagacgaccacctgaaatgcactggtatttatttctgataattaaaaattacaggggagggaagaactagaaaaagaacaactttagaccaaaggtgtctgagaaaaggagaaagggagcttgttcttcccattgctctttgtgatttaaggcaaaacagattaaaaaaaaaatctgcagc

SEQ ID NO: 190 - DNA, 110 bp, homo sapiens
tttgctccaagaatatcctagccatccttggcttctcctctatcatagctgtgatagctttgcttgctgtggggttgacccagaacaaagcattgccagaaaacgttaag

SEQ ID NO: 191 - DNA, 34 bp, homo sapiens
tgccagaaaacgttaagtatgggattgtgctgga

SEQ ID NO: 192 - DNA, 146 bp, homo sapiens
gtcctggaatctcaaaatttgttcagaaagtaaatgaaataggcatttacctgactgattgcatggaaagagctagggaagtgattccaaggtcccagcaccaagagacacccgtttacctgggagccacggcaggcatgcggttg

SEQ ID NO: 193 - DNA, 34 bp, homo sapiens
agaatgcagggttaaaggtcctggaatctcaaaa

SEQ ID NO: 194 - DNA, 81 bp, homo sapiens
ggtgccaggatcattactggccaagaggaaggtgcctatggctggattactatcaactatctgctgggcaaattcagtcag

SEQ ID NO: 195 - DNA, 123 bp, homo sapiens
gttgcaagtaatgaaattctcagggacccatgctttcatcctggatataagaaggtagtgaacgtaagtgacctttacaagaccccctgcaccaagagatttgagatgactcttccattccag

SEQ ID NO: 196 - DNA, 135 bp, homo sapiens
tttgaaatccagggtattggaaactatcaacaatgccatcaaagcatcctggagctcttcaacaccagttactgcccttactcccagtgtgccttcaatgggattttcttgccaccactccagggggattttggg

SEQ ID NO: 197 - DNA, 34 bp, homo sapiens
tggccaaggacattcaggttgcaagtaatgaaat

SEQ ID NO: 198 - DNA, 114 bp, homo sapiens
gcattttcagctttttactttgtgatgaagtttttaaacttgacatcagagaaagtctctcaggaaaaggtgactgagatgatgaaaaagttctgtgctcagccttgggaggag

SEQ ID NO: 199 - DNA, 138 bp, homo sapiens
ataaaaacatcttacgctggagtaaaggagaagtacctgagtgaatactgcttttctggtacctacattctctccctccttctgcaaggctatcatttcacagctgattcctgggagcacatccatttcattggcaag

SEQ ID NO: 200 - DNA, 521 bp, homo sapiens
atccagggcagcgacgccggctggactttgggctacatgctgaacctgaccaacatgatcccagctgagcaaccattgtccacacctctctcccactccacctatgtcttcctcatggttctattctccctggtccttttcacagtggccatcataggcttgcttatctttcacaagccttcatatttctggaaagatatggtatagcaaaagcagctgaaatatgctggctggagtgaggaaaaaaatcgtccagggagcattttcctccatcgcagtgttcaaggccatccttccctgtctgccagggccagtcttgacgagtgtgaagcttccttggcttttactgaagcctttcttttggaggtattcaatatcctttgcctcaaggacttcggcagatactgtctctttcatgagtttttcccagctacacctttctcctttgtactttgtgcttgtataggttttaaagacctgacacctttcataatctttgctttataaaagaacaatattga

SEQ ID NO: 201 - DNA, 34 bp, homo sapiens
tatggaagatacaaaggagtctaacgtgaagaca

SEQ ID NO: 202 - DNA, 128 bp, homo sapiens
agtctaacgtgaagacattttgctccaagaatatcctagccatccttggcttctcctctatcatagctgtgatagctttgcttgctgtggggttgacccagaacaaagcattgccagaaaacgttaag

SEQ ID NO: 203 - DNA, 151 bp, homo sapiens
gtcctggaatctcaaaatttgttcagaaagtaaatgaaataggcatttacctgactgattgcatggaaagagctagggaagtgattccaaggtcccagcaccaagagacacccgtttacctgggagccacggcaggcatgcggttgctcag

SEQ ID NO: 204 - DNA, 138 bp, homo sapiens
cagtttgaaatccagggtattggaaactatcaacaatgccatcaaagcatcctggagctcttcaacaccagttactgcccttactcccagtgtgccttcaatgggattttcttgccaccactccagggggattttggg

SEQ ID NO: 205 - DNA, 760 bp, homo sapiens
gccgcggacactgcagtgggctgggcgctcggctacatgctgaacctgaccaacctgatccccgccgacccgccggggctgcgcaagggcacagacttcagctcctgggtcgtcctcctgctgctcttcgcctccgcgctcctggctgcgcttgtcctgctgctgcgtcaggtgcactccgccaagctgccaagcaccatttaggggccgacgggggcagctgccccatccctcccccaacccctgtatccccaccccgtactcccacccctcccacaacccctgtacctcccacccctgtatccccacccctccacccacccctctcccaacctctctccccgcccctgtatcctgcattcctccacccaccctctatcccccaccgctccaccccaccactgtcttctccatccttccaccccaccctcagcgtctctgcccctaaggcagcccaggaaataggaactgagactctggtacccacaggagcctgggtgggcaaagagcgctcaatccagctccttgaacccctccagcccgcttcagcctgggcatcactgcaggccccgtgctcctcctcctcctcctcagggctgggtctccagagagtggggccttggtcctgagaatcagcccttagaggctccttctgtgtagtctgggtctgtactggggagggtcacagcccacgggctggcagccagcccagcacctacttgtaaaaattttgtaataaaaagtttttcctagagacgtg

SEQ ID NO: 206 - DNA, 234 bp, homo sapiens
aagaacctgtggcacatgtgggtgcacccgcatggagtggaaaccacgggtgccctggacttaggtggtgcctccacccaaatatccttcgtggcaggagagaagatggatctgaacaccagcgacatcatgcaggtgtccctgtatggctacgtatacacgctctacacacacagcttccagtgctatggccggaatgaggctgagaagaagtttctggcaatgctcctgcag

SEQ ID NO: 207 - DNA, 111 bp, homo sapiens
gcttttgcaggattctactacacagccagtgctttaaatctttcaggtagcttttccctggacaccttcaactccagcacctggaatttctgctcacagaattggagtcag

SEQ ID NO: 208 - DNA, 86 bp, homo sapiens
agctcgggcctgcgtctgcactatgtctcggctggacgaggtaacggacccctcatgctgtttctgcacggcttccctgagaactg

SEQ ID NO: 209 - DNA, 77 bp, homo sapiens
tggctacggatcatcacctaccttttcccagacggacagagaacatggttcaaaaacaagtgcaaaggccctttatg

SEQ ID NO: 210 - DNA, 159 bp, homo sapiens
ttgactacctttgtcctggatcggaaagatgctatgatcactgttgatgatggaataaggaaattgaaattgcttgatgccaagggcaaagtgtggactcaagatatgattcttcaagtggatgacagagctgtgagcctgattgatttagaatcaaag

SEQ ID NO: 211 - DNA, 150 bp, homo sapiens
aatgaactggagaattttcctttaaacacaatccagcactgccaagctgtgatgcattcatgcagctatgattcagttcttgcactggtgtgcaaagagccaacccagaacaagccagatcttcatctcttccagtgtgatgaggttaag

SEQ ID NO: 212 - DNA, 137 bp, homo sapiens
gatgatttccaatgcagaccctagtataccgcctccacccagagctcctgcccctgcgccccctgggaccgtcacccaggtggatgttagaagtcgagtggcagcctggtctgcatgggcagccgaccaaggggact

SEQ ID NO: 213 - DNA, 74 bp, homo sapiens
ttgagaaaccaaggcagtatcatgagcaggaagaaacacctgagatgatggcagcccgcattgacagagatgtg

SEQ ID NO: 214 - DNA, 75 bp, homo sapiens
gccaaactgaagtctcatattcagaatcctagtgctgcagatttggttcactttttgtttactccattaaatatg

SEQ ID NO: 215 - DNA, 149 bp, homo sapiens
gtggtgcaggcaacaggaggtcctgaactagccagttcagtacttagtcccctattgaataaggacacaattgatttcttaaattatactgtcaatggtgatgaacggcagctgtggatgtcattgggaggaacttggatgaaagccag

SEQ ID NO: 216 - DNA, 184 bp, homo sapiens
agcagagtggccaaaagaacagtttattccaccatatgttccacgattccgcaatggctgggagcccccaatgctgaactttatgggagccacaatggaacaagatctttatcaactggcagaatctgtggcaaatgtagcagaacatcagcgcaaacaggaaataaaaagattatccacagag

SEQ ID NO: 217 - DNA, 134 bp, homo sapiens
cattccagtgtatcagagtatcatccagccgatggctatgcgttcagtagcaacatttacacaagaggatcccacctggaccaaggggaagctgctgttgcttttaagccaacttctaatcgccatatagatag

SEQ ID NO: 218 - DNA, 109 bp, homo sapiens
aaattatgaaccactcaaaacacaacccaagaaatatgccaaatccaagtatgactttgtagcaaggaacaacagtgagctctcggttctaaaggatgatattttagag

SEQ ID NO: 219 - DNA, 144 bp, homo sapiens
atacttgatgatcggaagcaatggtggaaagttcgaaatgcaagtggagactctggatttgtgccaaataacattttggatattgtgagacctccagaatctggattggggcgtgctgatccaccttatactcatactatacag

SEQ ID NO: 220 - DNA, 223 bp, homo sapiens
aaacaaaggatggagtatggcccaagaccagctgatactccccctgctccatcacctcctccaacaccagctcctgttcctgttccccttcccccttccactccagcacctgttcctgtgtcaaaggtcccagcaaatataacacgtcaaaacagcagctccagtgacagtggtggcagtatcgtgcgagacagccagagacacaaacaacttccggtggacc

SEQ ID NO: 221 - DNA, 181 bp, homo sapiens
gaaggaaatctcagatggaggaagtgcaagatgaactcatccacagactgaccattggtcggagtgccgctcagaagaaattccatgtgccacggcagaacgtgccagttatcaatatcacttacgactccacaccagaggatgtgaagacgtggttacagtcaaagggattcaaccctgt

SEQ ID NO: 222 - DNA, 1269 bp, homo sapiens
cccatgcaaacctgagtgagcacaggatttgcttctaggcccattatttttattaaaactgaaaaaatttaaactgaattttttgaccttggaaaatatttttcttactttaccaaggtgaagtttccttaattagactaattattttatccccatcccagggtataaacaggaattgttttgatagtggtggagttattcactgcaacaaagcaacaatgttgtccatgattcaaaatctaagcagtttcgattttgcctgtgaatatggtgtctgtcattcagggcatagctcactgtaggctagcctctgcttacttaagtctcttctctgacatactcaatggaagaatatttagatttatttaaagttcttaatgccaacagtttaaaaaaaaattaaaacatttgaatgaactgtaaagtacagccataccttggacatgcaaatataaatctatggagcattctcaagagagtttgtcatggctctgttgattgcaactccttgtatagcttgtattttgatttagtttatattctgcttattatgtatactgtgttcttatatatgagaaagcacaaatgcgaaagaggtcatgtcttctcaaaatctagcaaaggaagtagtctgcattggtgtgcattacagtattttgcttaatgaaagcctcagttctgaatgttgatatgagtagttaaaaggaagtggggccattttatgtgtttatctgtgtcaagtatttctggtaataagaagcacttaatttacacatattttaatcctgtgaaagattccacatagagaaaagaaagatacctaaccttcaacaaatgttatttttggaaacacaatttttgtcattaaatgttatattatttcacatatataaaacagatgttatgtaagaatgttgtatattttaacataaatcatttagagaaattatctagattcattaattttcatagtgcctttttcacatgagtcagctggaaagtctgcaataaacagtatttgctgtctgttaagatagcagtggcagctccgagttacaagaaattatgagaagacgacaggaaaaaatcagtgctgccgctagtgattcaggagtggaatcttttgatgaaggaagcagtcactaatttgtttgtttgtatttaaactccattgtttttggcattattccaacatgctttgttttaagaagccttgaagggaatgtcagattcatttttcttgatgtaatttatcaccataaaaaaaaa

SEQ ID NO: 223 - DNA, 162 bp, homo sapiens
cacttgactacctttgtcctggatcggaaagatgctatgatcactgttgatgatggaataaggaaattgaaattgcttgatgccaagggcaaagtgtggactcaagatatgattcttcaagtggatgacagagctgtgagcctgattgatttagaatcaaag

SEQ ID NO: 224 - DNA, 34 bp, homo sapiens
agatacacacctcaaaggtactccctcctcccgg

SEQ ID NO: 225 - DNA, 337 bp, homo sapiens
tttgttgctcagcccaactgccaacagttgcttgccaccctgtggtatgatggcttccctggatggcggcggaaacactgggtagtcaagcttctaacctgcatgaccattgggttcctgtttcccatgctgtctatagcctacctgatctcacccaggagcaaccttgggctgttcatcaagaaaccctttatcaagtttatctgccacacagcatcctatttgaccttcctctttatgcttctcctggcttctcagcacattgtcaggacagaccttcatgtacaggggcctcccccaactgtcgtggaatggatgatattgccttgggttctag

SEQ ID NO: 226 - DNA, 140 bp, homo sapiens
gtttcatttggggtgagattaaggaaatgtgggatggtggatttactgaatacatccatgactggtggaacctgatggattttgcaatgaactccctctacctggcaactatttccctgaagattgtggcctatgtcaag

SEQ ID NO: 227 - DNA, 1635 bp, homo sapiens
tgaccatcctcagaatctcatgccattttcccgcagagctgagggatgtggtcagcagtccccaccagctatcagataaggcaaactaccctgcccatctttgttgccattcagtgctacgcgctgcccatgctgggtcaatgaacagcatcaaagggaggctgaagagatttcaaagaattgtcagcttgtggtttctgggtataagtggcttgtttcgttcttaaatactacaacttcttattgtgggggctattgagttttcttgtttcttaaagatgtgttagtttcctaatgtgtgccaatgaaatatttaccccatagtatatgtcatatctaataagttaggcgaattatttttctgtttgtgtgtttcagttaacttactacatttgaaaatttgatagtatcaaaattagtatttagaatttgtgctattttttaaaaacatctactacactctaaccaatagtcactacatctcatacattggcaagataaatagttgtcatatttgttgggtcctattctactgttgatagttttaattatcattgctatcaaataatttctaagataaaagcctgaatttgaaatatttttatcagaaaaatggcatattgctgaagtgtcaaaatatgagaaaagagcatcttcattttaattaactgctttataaaaatggcaactttaggtatagataacctaattcaggttgcagttttaatattcattgggggcagtggaattgcatgtgtaaattatgttagatatatatcataacaaccctagtggattcatggaattataaacgtgatccaaattctactgtcttccaagaaagctacatttatacatccaaacaaattcttttccacagcttaagagtacctcatcagaatacagtggttcaagttaaaaaataaagtccataatgcatatggtcatcattctagacctgataaccactgagaacctgctgagcaaggacggcttttaccaacattagcacaaaggtttcctcagtttagactctaacatatgacctgaaacaactctaggtctgagtcctagagattacataaatagtgtgtagcccagaaaagtaaaataggtgtcacgctgttttttttaatgtcaacaccaataggaatccatttagattaatgtatgtaatttctgataaaagtaagagaaatgaggaactttcgaactgcaaatatgcctccaaactagcttttgataagtgtggtttctgttatttgaatggattcccatctttgtctctagcctggagaagagcaaaatggagggggaggggccagaaataggaggaggttttgaagaataaagcctcatatccaggaaaaagtgatcaagtgaaaaagctgcatcaagacccatttggcaagaggttgtccgaaaataattccttgatcatatctgaagtggttatttactcaaatcaatttctcccctgcttcgctaagactgtcataaaatggccttgacatttaatcacaggttactttccagaaaggtttctaaagttacagattcagcttttcaaattgtcaaattcactttgaataataatagaacctgggctacattcctaactcatattaacagcac

SEQ ID NO: 228 - DNA, 248 bp, homo sapiens
atttcgccgccagctttctgaaccctgtaactcctttcctcctttgccgacgatgccaagggaaggacgtcctatgtaccaacgccagatgtctgagccaaacatccccttcccaccacaaggctttaagcaggagtaccacgacccagtgtatgaacacaacaccatggttggcagtgcggccagccaaagctttccccctcctctgatgattaaacaggaacccagagattttgcatatgactcag

SEQ ID NO: 229 - DNA, 28 bp, homo sapiens
tcagtcaacttcaagaggcttggttagc

SEQ ID NO: 230 - DNA, 136 bp, homo sapiens
ggtgcgcggagcggttcaccgtcttcggagcggttcggcccagcctttcgcccaggcgcccaggcccgctgcgcgcgtgcgtgagcgcgcctgcgccgccggggccgctgcaaggggaggagagaggccgcctcag

SEQ ID NO: 231 - DNA, 119 bp, homo sapiens
gaggatcccttttcccccagaaattactcaatgctgaaacctctcaaagtggtattagagacgctgaaagcaccatggacgggttttatgatcagcaagtcccttttatggtcccaggg

SEQ ID NO: 232 - DNA, 88 bp, homo sapiens
aaatctcgatctgaggaatgcagagggcggcctgtgattgacagaaagaggaagtttttggacacagatctggctcacgattctgaag

SEQ ID NO: 233 - DNA, 48 bp, homo sapiens
agctatttcaggatctcagtcaacttcaagaggcttggttagctgaag

SEQ ID NO: 234 - DNA, 51 bp, homo sapiens
cacaagttcctgatgatgaacagtttgtcccagattttcagtctgataacc

SEQ ID NO: 235 - DNA, 130 bp, homo sapiens
tggtgcttcatgccccacctccaaccaagatcaaacgggagctgcacagcccctcctctgagctgtcgtcttgtagccatgagcaggctcttggtgctaactatggagaaaagtgcctctacaactattg

SEQ ID NO: 236 - DNA, 1494 bp, homo sapiens
aaaaacgaaaaacaaaaaacaaaaacaaaccttttggtaactgtgcactgtgtcctttagccagggccgtgccaacttatgaagacactgcagcttgagaggggctttgctgaggcttccccttggccatgtgaaagcccgccttgttgcctgctttgtgctttctgcaccagacaacctgatggaacatttgcacctgagttgtacatttttgaagtgtgcagggcagcctggacacaagcttagattctctatgtatagttccccgtgttcactaacatgccctctctggaaagcatatgtatataacatgtgtcatgtcctttggaaacctggtcacctggtgaaaacccttgggattcttccctgggcatgactgatgacaatttccatttcatcagtttgttttgttttcctttttctttaaatcttggactttaaaccctacctgtgtgattcagtagggtttgagacttacgtgtgatactgacaggtaagcaacagtgctagcattctagattcctgcctttttttaaaaagaaattattctcattgctgtattatattggaaaagttttaaacaaccaagctaaagctatgtgaaagttgagctcaaagtagaggaaaagttactggtggtaccttgctgcctgctctgctggtagaattctgtgctccccgtgacacttagtacattaagaatgactacactgttcctcgtatgtgaaggaggcagtgctgactccgtgagtgtgagacacgtgctttgaactgcttttctattcatggagcactccatagtctcaaactgtcccccttatgaccaacagcacatttgtgaagaggttcgcagggataaggggtgcactttatagctatggaaacatgagattctcctctattggaagctaattagcccacaaaggtggtaaacctgtagattgggccttaattagcattgtactctaatcaaaggactctttctaaaccatatttatagctttcttaacctacacatagtctatacatagatgcatattttacccccagctggctagagatttatttgttgtaaatgctgtatagatttggttttcctttctttacttaccctggtttggattttttttttttttcttttgaatggatttatgctgtcttagcaatatgacaataatcctctgtagcttgagctacccctcccctgctgtaacttacgtgacctgtgctgtcactgggcataggacagcggcatcacggttgcattcccattggactcatgcacctcccggatggtttttgtttttttcgggggttctttggggtttgtttgtttgcttcttttccagagtgtggaaagtctacagtgcagaaaggcttgaacctgccagctgatttgaaatactttcccctgcgcagggccgtatgcatcctgccaagctgcgttatattctgtactgtgtacaataaagaagtttgcttttcgtttaccaa

SEQ ID NO: 237 - DNA, 222 bp, homo sapiens
gaatgcgtcatgaaaggcgtcacttccacgagagtttatgagagagcataagccaagggacgttgacctggactgaagttcgcattgaactctacaacattctgtgggatatattgttcaaaaagatattgttgttttccatgatttagcaagcaactaattttctcccaagctgattttattcaatatggttacgttggttaaataaactttttttagatt

SEQ ID NO: 238 - DNA, 2546 bp, homo sapiens
cccccaccatgccgtggcccctgctgctgctgctggccgtgagtggggcccagacaacccggccatgcttccccgggtgccaatgcgaggtggagaccttcggccttttcgacagcttcagcctgactcgggtggattgtagcggcctgggcccccacatcatgccggtgcccatccctctggacacagcccacttggacctgtcctccaaccggctggagatggtgaatgagtcggtgttggcggggccgggctacacgacgttggctggcctggatctcagccacaacctgctcaccagcatctcacccactgccttctcccgccttcgctacctggagtcgcttgacctcagccacaatggcctgacagccctgccagccgagagcttcaccagctcacccctgagcgacgtgaaccttagccacaaccagctccgggaggtctcagtgtctgccttcacgacgcacagtcagggccgggcactacacgtggacctctcccacaacctcattcaccgcctcgtgccccaccccacgagggccggcctgcctgcgcccaccattcagagcctgaacctggcctggaaccggctccatgccgtgcccaacctccgagacttgcccctgcgctacctgagcctggatgggaaccctctagctgtcattggtccgggtgccttcgcggggctgggaggccttacacacctgtctctggccagcctgcagaggctccctgagctggcgcccagtggcttccgtgagctaccgggcctgcaggtcctggacctgtcgggcaaccccaagcttaactgggcaggagctgaggtgttttcaggcctgagctccctgcaggagctggacctttcgggcaccaacctggtgcccctgcctgaggcgctgctcctccacctcccggcactgcagagcgtcagcgtgggccaggatgtgcggtgccggcgcctggtgcgggagggcacctacccccggaggcctggctccagccccaaggtggccctgcactgcgtagacacccgggattctgctgccaggggccccaccatcttgtgacaaatggtgtggcccagggccacataacagactgctgtcctgggctgcctcaggtcccgagtaacttatgttcaatgtgccaacaccagtggggagcccgcaggcctatgtggcagcgtcaccacaggagttgtgggcctaggagaggctttggacctgggagccacacctaggagcaaagtctcacccctttgtctacgttgcttccccaaaccatgagcagagggacttcgatgccaaaccagactcgggtcccctcctgcttcccttccccacttatcccccaagtgccttccctcatgcctgggccggcctgacccgcaatgggcagagggtgggtgggaccccctgctgcagggcagagttcaggtccactgggctgagtgtccccttgggcccatggcccagtcactcaggggcgagtttcttttctaacatagccctttctttgccatgaggccatgaggcccgcttcatccttttctatttccctagaaccttaatggtagaaggaattgcaaagaatcaagtccacccttctcatgtgacagatggggaaactgaggccttgagaaggaaaaaggctaatctaagttcctgcgggcagtggcatgactggagcacagcctcctgcctcccagcccggacccaatgcactttcttgtctcctctaataagccccaccctccccgcctgggctccccttgctgcccttgcctgttccccattagcacaggagtagcagcagcaggacaggcaagagcctcacaagtgggactctgggcctctgaccagctgtgcggcatgggctaagtcactctgcccttcggagcctctggaagcttagggcacattggttccagcctagccagtttctcaccctgggttggggtcccccagcatccagactggaaacctacccattttcccctgagcatcctctagatgctgccccaaggagttgctgcagttctggagcctcatctggctgggatctccaaggggcctcctggattcagtccccactggccctgagcacgacagcccttcttaccctcccaggaatgccgtgaaaggagacaaggtctgcccgacccatgtctatgctctacccccagggtagcatctcagcttccgaaccctgggctgtttccttagtcttcattttataaaagttgttgcctttttaacggagtgtcactttcaaccggcctcccctacccctgctggccggggatggagacatgtcatttgtaaaagcagaaaaaggttgcatttgttcacttttgtaatattgtcctgggcctgtgttggggtgttgggggaagctgggcatcagtggccacatgggcatcaggggctggccccacagagaccccacagggcagtgagctctgtcttcccccacctgcctagcccatcatctatctaaccggtccttgatttaataaacactataaaaagttctttgcttta

SEQ ID NO: 239 - DNA, 172 bp, homo sapiens
gctgtctacaaggcctggctgtgctcagaatacttcagcgtgacccagcaggaatgccagcgctgggtgccctgcaagcaatactgcctggaggtgcagacccggtgcccctttatactccccgacaatgaggaaatggtgtacggagggctccctggctttatctgtacag

SEQ ID NO: 240 - DNA, 2456 bp, homo sapiens
ggttgctggatacttcaccaaagcgtctggaaaccaagtgctgtgacgtgcagtgggtctcctgtgaggcgaagaagaagaagttcaaggagtctgaggcccccaaaacccaccagcagcaattccaccactcctatttccaccactaccaccaacagtaccatcactaccacccccatcatgatcccccaggccgtgtcagcaacaagcccgccctgctgccggtctctgggggctcccgcctcagccctagcaggatccggctctgcgtccttgttctcatgctcctccataccgtggtgtccttctccagcaaccagggtggtgggggattggggctggagacactgcctgccctagaggagggcctgacacgggaagagtgacagtagggagggaggacagacctccaccacactgacatcagctccagctcccccaggttgggggggagggggctcctcccatgggaggtgtaggataaggtgggggcgggggaaatgggggaatgacacatcccccccaacctacccccaccccaaaagcagctccaacagaatggccagagggcctcagggagctgcaaaactcatccaggagaaaaaggcaggagcagcaagtgacccctcccaagctcccatctatggggcttagcaaaaaaagggaggaagtgggggctggatatgcccacccctgccaaaagccctgaccccagggaaggaggctgctcacccagccttggccctgcagggaatgttggggggcacagaggggagaagctctttcccccactccacattccttttgttgccaagatccttaattccctcctgcccatatccctacaggcgacggcagacagtgcaatggccctcctgccacttcagcacaccttgccccacctgggaccatcacacgtgaagcaccaggctgggagatgaggtgcacacagttgcagctaggtcggggccccagttaagctgtgccccaccaccctaggcaatgaggggcaggaaaggggtacagaatgaatggtgaaagagagtggagatacggaagggggagaggagaggagaaatgtggacagagggcttgaagacatggccgaataggctactgccacccggctttggggagatgggcgataagtgttgaggtaggctcgagaactgctccccaaatcagtgagaattgcattaggagccctctggggaaagagcacaggaactgagttgctgggcctcaaaacaaaaggctgcaggcacatggttagactgccgctgtctgaaggaccatatcttacagaaggtgcacatactcccaagggccactcttgccctggagatcttggccttggggccaaagacatttccatttccgatctccatggggaggagggacttaagcccaagtgggtcaggcctggcccaggtccttatactcccctatcttgtcctagcccaggcctccatgaaggagaacctggtggcattgccccagagctctccagagatgagcctcccctatccccaccctgtccccgtccccagcctaccaaagagtattcacacctttccatccctgactctatgggttactgtcccaattgcgaaacacccatactcctcttcaccctgtagcagggtcttctgctccagtcaccccggcctcttgcaagagaaagtccagggactggcaagaagaccctttgactaaagataatgtgctgtcatatcttggctgagaaactgttgagtcaggctagaggactggacaagagaagaggcattgagagcccccttgggagccattagccttgctctggtctgctgtgtggggttggggagtaggggggatcccacattgcctgggacatctgaatgctgatacgaatggacaggaaagggagccagagctgtatacctgggacaggccagggagcctttagtgccagcagggcctgtgtcctgggatgtgatgctagtgagtgaatgggagggcgggcctggagctgcgtgagagtgaaaccaagaagcggatggggaagggagagctggccttggtcagcttagggatgaatggagaaggaatcattcatgggcctcggctaggtcccagactgattacatgagactggggagtccttccctgccagttttctcatttattacagcctatcccccaaccctacccctgtgccctgcctacacactcccgtttggccctcagcactttgggcctggtcacataccccatccctagagtgctcttttcagctggggaggggaagaggtgctggctcctttccagacatgcttatgtataagtaaaggggcaggtgcttgggctccagagagaccctaggtactgccttctaactcctgcatcctcagaaggggaaaagggactgggaagtaaagggaaaccatatggccccagggaatggacccttggag

SEQ ID NO: 241 - DNA, 117 bp, homo sapiens
gtgcttgggtaaatcatggtcggggaatggtcaaggaacataacaatcatttaatctggacatatgatgcaccacatttggggtactggatagcagctccacttccaggaactagag

SEQ ID NO: 242 - DNA, 203 bp, homo sapiens
gtgagctttgaatgggcatagacagggttcccagcccaatagtcgggattggggtataggttctacatgggaaagtatccagtctcttgctttactcagtgtcctgtgaccaccagaggcatgagagggacaacacactcttttcgctctaccacaaggggtgactagaagatgagcaagtctaataaaaagatacggcctta

SEQ ID NO: 243 - DNA, 272 bp, homo sapiens
tggcagggccatggcggaggcccgagtctcccagcctggggcatctccacgctctgtaacgctgagctccaggcacccgtgaagccccacgggtcaaggctggtgggccggggctgggaggcctgcacgcctgggttctgggtccctaaaccagtacccatccaccacagccaccatgatctggcttcgaaacaggaggtgccttgagccgctccagggcaccccgaagtgggtccctgttctgggggagctgcaaaagaccctccagaagg

SEQ ID NO: 244 - DNA, 349 bp, homo sapiens
gatgaaacgaggaggaagagatagtgaccgtaattcatcagaagaaggaactgcagagaaatccaagaaactgaggactacaaatgagcattctcagacttgtgattggggtaatctccttcaggacattattctccaagtatttaaatatttgcctcttcttgaccgggctcatgcttcacaagtttgccgcaactggaaccaggtatttcacatgcctgacttgtggagatgttttgaatttgaactgaatcagccagctacatcttatttgaaagctacccatccagagctgatcaaacagattattaaaagacattcaaaccatctacaatatgtcagcttcaag

SEQ ID NO: 245 - DNA, 96 bp, homo sapiens
aatgaatatgaacagttaaattatgcaaaacaactgaaagagagattggaggcttttacaagagattttcttcctcacatgaaagaggaagaggag

SEQ ID NO: 246 - DNA, 34 bp, homo sapiens
aattccccaatgaccagggttcgcagccaggcca

SEQ ID NO: 247 - DNA, 88 bp, homo sapiens
ccggacacacgggggcacagaatcactgaaggggaaaaatacagaaaatatgggtttctacggcacattaaaaatgattttttacaaa

SEQ ID NO: 248 - DNA, 1374 bp, homo sapiens
atggagaacgttgataattctctggatggcagtgatgtgtcagaaccagcaaaaccagaagccggacttgaagtggctcagtcgatcctgagtaagttctctatgaaatccctatttgggtttacgagtaaattagaatctgtgaatccagaagaggaagatgctgtgctgaaagcattccatagtttagacgtgaatcccacatctcagcaggatgattccagcaatggcttggatccacaggaagcagggtctcgggtttcacctgacctcggaaatgatgagaaaattgcaagcgtggagacagaatcagagggaagtcagagaaaagaagcagggacatctctccttgctcaagagctgcttccactgtctactttgaaagggacaaaagacgatgtcatttgtgttcgtgggaccctggtgcacaccaccagtgattctgactctgatgatggtggccaggagccagaggagggaagcagcaccaatggcccaaagtcccctagtggtgttttatctgagccatctcaggagtccaaagaaaacccaggaggtttcagggaaaatactgtcactggggaaatgaatggtgcagagctctgtgcagaagatcctcaaaggattccacctgagatgagcagcaaactggaagctggcaacggtggtcttcagacagagcgtcgccccagccaggaccaggtgggagaagaagggtcccaagacctacctgcagtaacaaaccagaattcgagtgttggtatcactgagagtgcctcttctaaaaaagaagtctctggagagaagtcattccaacttccagcttttttcagtgggcttcgtgtgctgaagaagggggctaccgctgagggaggagagaccatcacggaaattaaaccaaaggatggggacttagctttgctcaaattgacgcagcctgtgcagaagtccttagtgcaggcagggctgcagacagtgaagagtgagaagaaagcaactgatccgaaggccactcccactctcctggagcagctctctctgctgctcaacattgacatgcccaaaactgaaccgaagggagcagaccctgagtcgcccaggagagaagagatgggctgcaatgctgaccaagagagccagagcggccctggagtaccacaaacccagggtggtgaggtcaagccaaagtcgccagagacagccctggaggcttttaaagccctattcatccggccccccagaaaggggaccacagctgacacctctgagctggaagctctcaagcgcaagatgagacatgagaaggagtcgctaagagctgtgtttgaacggtccaattctaagccagcggatggcccctctgattccaaaagt

SEQ ID NO: 249 - DNA, 200 bp, homo sapiens
gcgagcaccgcgccggccctgagcctcccgctcgctccccacggccgcggtgcatgttcgcctcctgccactgtgtgccgagaggcaggaggaccatgaaaatgatccactttcggagctccagcgtcaaatcgctcagccaggagatgagatgcaccatccggctgctggacgactcggagatctcctgccacatccag

SEQ ID NO: 250 - DNA, 105 bp, homo sapiens
agggaaaccaaagggcagtttctcattgaccacatctgcaactactacagcctgctggagaaggactactttggcattcgctatgtggacccagagaagcaaagg

SEQ ID NO: 251 - DNA, 79 bp, homo sapiens
ctcatccaccatacaccatgtgctttagagtgaaattctacccacatgaacccttgaagattaaagaagagctcacaag

SEQ ID NO: 252 - DNA, 34 bp, homo sapiens
cttcaagcaaatgaaaactcatccaccatacacc

SEQ ID NO: 253 - DNA, 98 bp, homo sapiens
ataccttttataccttcagattaaaagggacatttttcatggccgcctgctgtgctccttttctgatgctgcctacctgggtgcctgtattgttcaag

SEQ ID NO: 254 - DNA, 123 bp, homo sapiens
ctgagcttggtgattacgatcctgatgagcatcctgagaattacatcagtgagtttgagattttccccaagcagtcacagaagctggaaagaaaaatagtggaaattcataaaaatgaactca

SEQ ID NO: 255 - DNA, 88 bp, homo sapiens
ggggcagagcccaccagttgctgaatttaacttgctcctgaaagctcacactttggaaacctacggggtggatcctcacccatgcaag

SEQ ID NO: 256 - DNA, 89 bp, homo sapiens
gattcaacaggcacaacaacatttttaggattcacagctgcaggctttgtggtctttcagggaaataagagaatccatttgataaaatg

SEQ ID NO: 257 - DNA, 34 bp, homo sapiens
atcctcacccatgcaaggattcaacaggcacaac

SEQ ID NO: 258 - DNA, 89 bp, homo sapiens
aaaaaagccatgttggcattccatacttcaacaccagctgcctgcaaacatctttggaagtgtggagtggaaaaccaggccttttataa

SEQ ID NO: 259 - DNA, 75 bp, homo sapiens
gtatgcaaaatccagtcagatcaagactgtatcaagcagcaagatattttttaaaggaagtagatttcgatatag

SEQ ID NO: 260 - DNA, 34 bp, homo sapiens
aaccaggccttttataagtatgcaaaatccagtc

SEQ ID NO: 261 - DNA, 69 bp, homo sapiens
tgggaaagttgccaaagaggtggtggaggccagttccaagatccagagggagcctcctgaggtgcacag

SEQ ID NO: 262 - DNA, 34 bp, homo sapiens
agtagatttcgatatagtgggaaagttgccaaag

SEQ ID NO: 263 - DNA, 125 bp, homo sapiens
agccaacattactcagagccgcagttcccactccttgaacaaacagctcatcattaacatggaacccctgcagcccctgcttccttcccccagcgagcaagaagaagaacttcctctgggtgagg

SEQ ID NO: 264 - DNA, 34 bp, homo sapiens
cctcctgaggtgcacagagccaacattactcaga

SEQ ID NO: 265 - DNA, 462 bp, homo sapiens
gtgttccattgcctaaagaggagaacatttctgctcccttgatctccagctccccagtgaaggcagcccgggagtatgaagatccccctagtgaagaggaagataaaataaaagaagaacctttaaccatctctgaactagtgtacaacccaagtgccagcctgctccccacccctgtggatgacgatgagattgacatgctctttgactgtccttctaggcttgagttggaaagagaagacacagattcatttgaggatctggaagcagatgaaaacgcctttttgattgctgaagaagaggagctgaaggaggctcgccgtgctttgtcgtggagctatgacattctgactggccatattcgggtgaacccactggtcaagagtttttccaggctccttgtggtgggcctgggactgctgctctttgtatttcccctgctcctcctccttttggagtcag

SEQ ID NO: 266 - DNA, 660 bp, homo sapiens
gtattgatctctccttcttatgcgaaatccgccagacaccagagtttgagcagtttcactatgaatactactgtcccctcaaggagtgggtggctgggaaagtccacctcatcctctacatgctgggttgctcatgaagttaatctctcacgtgactaagggctatattcaatgctagtgatttctttttttcagcaaatgcctggttctgaagggtcacggggctgtcaacaggtgttccttactcataattgattattcaaacctttaagttagctttccataattcactgcacttaaataagtttaaatcaaatacagttattttagttacaggttaggaagatggtctttaaataaccaaaaatatgtttattttttattatagtgtagacatacccttcatctattatatcataatacatgttacattggactgaattagattttcccatttctaatagttggcaccattataagctataaggttcagaatcagaattttagtaacaactcaagagaaagttgttgaatataatccttagtgaaaacagtgtcctctaaccaatgcctatacaactaaatttatgctgggtttttggttctgtttttttaaaaatatttttatgtgttcaaactattttggtaaatttttagcaa

SEQ ID NO: 267 - DNA, 34 bp, homo sapiens
cctccttttggagtcaggtattgatctctccttc

SEQ ID NO: 268 - DNA, 34 bp, homo sapiens
acttcctctgggtgagggtgttccattgcctaaa

SEQ ID NO: 269 - DNA, 124 bp, homo sapiens
ctgagcttggtgattacgatcctgatgagcatcctgagaattacatcagtgagtttgagattttccccaagcagtcacagaagctggaaagaaaaatagtggaaattcataaaaatgaactcag

SEQ ID NO: 270 - DNA, 1025 bp, homo sapiens
actatatgtcggaaaccaaagacctccactgatcgacacagcttgagcctcgatgacatcagactttaccagaaagacttcctgcgcattgcaggtctgtgtcaggacactgctcagagttacacctttggatgtggccatgaactggatgaggaaggcctctattgcaacagttgcttggcccagcagtgcatcaacatccaagatgcttttccagtcaaaagaaccagcaaatacttttctctggatctcactcatgatgaagttccagagtttgttgtgtaaagtccgtctgtgtgcagctgtacaggcagcttactgtttgctagaggatgcgaaagtcataagttctttacatattacttgtgccatatcttcttcaccctaaacatagctctttctttataatatttgtgatgatggaaacaaaagccttggaacaattgcactttaagtattacacagaagtaaaagaactacagaaaatgtacagcaagacaagtgcccggaagttcactgatccttcagaaggaaatgcgctttactgattgcaaagccttcagaatattggagtgtggtgtgtttgctcatctgatgctttttagttcagttacatgtaacatcacatttttttatcacgtgaaagatgttagatttgtttgcttataaattttttaccactcccacataaaatgctcatagtttgggagaggaaagagggaagattctctcttcttttaacagagagatgattgctctgtatacccattgcttcctccctgaggctgtcccaaagtgaacactgatggagtggtcaaaatcataaggttgtagcaagccaaagatacgtatgtgacagaagcacataagcaataagcagaaaaccagaagtgcatgctgtgatgcctgtgactccttcatcccgctcagtgccatgtcctcttttgtgatcttccagaaagctccaggattcatttgagttccacatccaagtaacagatgaattatattcatgttgtaatgca

SEQ ID NO: 271 - DNA, 2555 bp, homo sapiens
ggccactagcgcggcgccgccagccgggagccagcgagccgagggccaggaaggcgggacacgaccccggcgcgccctagccacccgggttctccccgccgcccgcgcttcatgaatcgcaagtttccgcggcggcggcggctgcggtacgcagaacaggagccgggggagcgggccgaaagcggcttgggctcgacggagggcacccgcgcagaggtctccctggccgcagggggagccgccgccggccgtgcccctggcagccccagcggagcggcgccaagagaggagccgagaaagtatggctgaggaggaggcgcctaagaagtcccgggccgccggcggtggcgcgagctgggaactttgtgccggggcgctctcggcccggctggcggaggagggcagcggggacgccggtggccgccgccgcccgccagttgacccccggcgattggcgcgccagctgctgctgctgctttggctgctggaggctccgctgctgctgggggtccgggcccaggcggcgggccaggggccaggccaggggcccgggccggggcagcaaccgccgccgccgcctcagcagcaacagagcgggcagcagtacaacggcgagcggggcatctccgtcccggaccacggctattgccagcccatctccatcccgctgtgcacggacatcgcgtacaaccagaccatcatgcccaacctgctgggccacacgaaccaggaggacgcgggcctggaggtgcaccagttctaccctctagtgaaagtgcagtgttccgctgagctcaagttcttcctgtgctccatgtacgcgcccgtgtgcaccgtgctagagcaggcgctgccgccctgccgctccctgtgcgagcgcgcgcgccagggctgcgaggcgctcatgaacaagttcggcttccagtggccagacacgctcaagtgtgagaagttcccggtgcacggcgccggcgagctgtgcgtgggccagaacacgtccgacaagggcaccccgacgccctcgctgcttccagagttctggaccagcaaccctcagcacggcggcggagggcaccgtggcggcttcccggggggcgccggcgcgtcggagcgaggcaagttctcctgcccgcgcgccctcaaggtgccctcctacctcaactaccacttcctgggggagaaggactgcggcgcaccttgtgagccgaccaaggtgtatgggctcatgtacttcgggcccgaggagctgcgcttctcgcgcacctggattggcatttggtcagtgctgtgctgcgcctccacgctcttcacggtgcttacgtacctggtggacatgcggcgcttcagctacccggagcggcccatcatcttcttgtccggctgttacacggccgtggccgtggcctacatcgccggcttcctcctggaagaccgagtggtgtgtaatgacaagttcgccgaggacggggcacgcactgtggcgcagggcaccaagaaggagggctgcaccatcctcttcatgatgctctacttcttcagcatggccagctccatctggtgggtgatcctgtcgctcacctggttcctggcggctggcatgaagtggggccacgaggccatcgaagccaactcacagtattttcacctggccgcctgggctgtgccggccatcaagaccatcaccatcctggcgctgggccaggtggacggcgatgtgctgagcggagtgtgcttcgtggggcttaacaacgtggacgcgctgcgtggcttcgtgctggcgcccctcttcgtgtacctgtttatcggcacgtcctttctgctggccggctttgtgtcgctcttccgcatccgcaccatcatgaagcacgatggcaccaagaccgagaagctggagaagctcatggtgcgcattggcgtcttcagcgtgctgtacactgtgccagccaccatcgtcatcgcctgctacttctacgagcaggccttccgggaccagtgggaacgcagctgggtggcccagagctgcaagagctacgctatcccctgccctcacctccaggcgggcggaggcgccccgccgcacccgcccatgagcccggacttcacggtcttcatgattaagtaccttatgacgctgatcgtgggcatcacgtcgggcttctggatctggtccggcaagaccctcaactcctggaggaagttctacacgaggctcaccaacagcaaacaaggggagactacagtctgagacccggggctcagcccatgcccaggcctcggccggggcgcagcgatcccccaaagccagcgccgtggagttcgtgccaatcctgacatctcgaggtttcctcactagacaactctctttcgcaggctcctttgaacaactcagctcctgcaaaagcttccgtccctgaggcaaaaggacacgagggcccgactgccagagggaggatggacagacctcttgccctcacactctggtaccaggactgttcgcttttatgattgtaaatagcctgtgtaagatttttgtaagtatatttgtatttaaatga

SEQ ID NO: 272 - DNA, 221 bp, homo sapiens
gattatacactcaccatgtatttccagcagtcttggaaagacaaaaggctttcttattctggaatcccactgaacctcaccctagacaatagggtagctgaccaactctgggtaccagacacctactttctgaatgacaagaaatcatttgtgcatggggtcacagtgaaaaatcgaatgattcgactgcatcctgatggaacagttctctatggactccg

SEQ ID NO: 273 - DNA, 68 bp, homo sapiens
gtccccccgtggctgtggggatgaacattgacattgccagcatcgatatggtttctgaagtcaatatg

SEQ ID NO: 274 - DNA, 221 bp, homo sapiens
gattataccttgacaatgtactttcaacaagcctggagagataagaggctgtcctataatgtaatacctttaaacttgactctggacaacagagtggcagaccagctctgggtgcctgatacctatttcctgaacgataagaagtcatttgtgcacggagtgactgttaagaaccgcatgattcgcctgcatcctgatggcaccgtcctttatggactcag

SEQ ID NO: 275 - DNA, 83 bp, homo sapiens
aatcacaaccacagctgcctgcatgatggacctaaggaggtacccactggatgaacaaaactgcaccttggaaattgagagct

SEQ ID NO: 276 - DNA, 517 bp, homo sapiens
atggacccccatgagaacatcttactgagcactctcgagataaaaaatgaaatggccacatctgaggctgtgatgggacttggagaccccagaagcacaatgctagcctatgatgcctccagcatccagtatcggaaagctgggttgcccaggcatagttttggccgaaatgctctggaacgacatgtggcgcaaaagaaaagtcgcctgaggagacgcgcctcccaactgaaaatcaccatccctgacttgactgatgtgaatgccatagatcggtggtcccgcatattcttcccagtggttttttccttcttcaacatcgtctattggctttattatgtgaactaaaacatggcctcccactggaagcaaggactagattcctcctcaaaccagttgtacagcctgatgtaggacttggaaaacacatcaatccaggacaaaagtgacgctaaaataccttagttgctggcctatcctgtggtccatttcataccatttgggttgcttctgctaa

SEQ ID NO: 277 - DNA, 5483 bp, homo sapiens
gtaatgaatacactaaggtccttgtggttttccagttaaaacgcaagtgatttttacacatggtggctagacagcctgagtgctccatgttgtctgtttagtatgcagcctactcagaggggaataatttggaagatattcttagaaggctcaacagcattgtcagtcatttggtcataaccagatactcctcatcttcctttttaaggtcggcatgctgttgagatggcattgtccttatgaaacattatctgtaatgccatgcaaacacattacttgaagatgatctatgattttcttactgagatgataatggtgtcatgggaagattattcccactggtttctaaaagattctcttgtgatttgagcaggctaggggaatcaagacagagactcaggaaaccgatttggccagtccctttgcttgagcacggagaatcaactatgatgtctttcattttggcatttagagatgaggagacttctggacaatatcatgcatggtggccaagaaaatttttgctaaacggctatttcctaggtggttaaagtaagagttaacaacacagagcagagtagaaaatcagaaacgacggttgtaacaaaaagcaagcaagcaagaaagcatgacccttatcagctccacacagcatctggtctatacatactgtgtctagtggcagagggtgagtaagagagattgtttttccaacatcactagcttttgacagcatacctaaagaggtttttatagtttaatcgaagataaccatgaaatacaaaaattagagaaatgagaattgtcttagatattcaggttagattcagagtcattcaaatatacaaatacagagcacaaactagcaaacagattgtttttctcagacgaagtcagctgtccctgcaatttttgtccctataattttatttcttaatttaatagagtatttcaagagaaggatttgttcatcactttaaaggattcatggtcctctccagctttttttttgcttccaaaattctgattatattcttaggagactggcaaagtaaggctttggagagattgagtgaactatattttcttatatgcaatgaaattcaccattgtatttttcaaagcaaataatacttcaaggagtatgaacacacagagagttaatttgtgcattcattggtttggggcttgattttcttaagagtgctttcattgtatatgttgcaatctgcctaatcaaggaaaatatgaaaactagtggaaaataataacaaagccacattaacatgataaatgttatgagcctcagttgatattgcagaaaaatatataactgttaatcaggcaggcatgatgtattttttttctatttttaagttatgccattgtgcttctttctacactaattggacataagcattcatatgttggacaagaggagaataagctaatatgtcttccttctatcaatataaggctcatattttgcttttcttatagaaaatagtgaattgctaaattttccttaaagttgaatttgattgaactttttcttggaggatgactttgctatttctattttgaaaacaaagaaagctcttctttttagagtaatatccctaattgtgcatagcagccagtatccacctgtgaataatgtatgttttcttgattccttgtttccttcatgatttagcagcttggaatatgatggagtactattgcccttgcattccagctgtccaagaacaacaacaaacctttatccttcagctttctgaatttgccccaaaatggaatggtattttgtttttcaccagtgtaggaaaccttggccattatattttgattacataatcttgagcacctctgagtatcatctttgggcagaatctgaccaagattcttctcacaggctcagagtagagattcctcatgtttctccaggacacactttctcctaggcaatcattttggggcatcacactaagtaaagtgcctgccccttgtcaacagggggtaaagtttgactaaaaaacatcaggaaggaacatattgataactcaggggcacaactccaaaatgccattctgaaaaggctcttctagagtcaactcagagcccaagtgtagaagtgggctcttatgtgcaatcactgtacaagccataaaaatctttgatattgccatttatgattacaacaaaattttagatgtccaagaaagtcaacagaacaatccagatcaaagtgtagagaagcatctttgcagcttgagcctcaaaagcataaaaccaggaattcatttctgctctcagtgatacaagaaacaaggcaaaggaaaaaagatgggaccttagaagcaatgccctagagtaaaaagcatacattggcactcagtaaccacagagatagaaacaactgactcagcctctccagaaataacagtagatgtagaatttgttcttaaatatatgtttcgttgaaatgaaatgcagtcatcaaatgcgaggaggggtgctagacctgataagtctaggaagaccagtcagcttgatagccagttgttaaactaaaggaccacaaaggtgtgtttagtagttctgatatgggattactatcagtttatacatatgtatagattttcttttaattataaacagccagatagccagcaattaaggtgttttttcatctttgtgaaattcatggacaaattctgactctaaaatgcacagtctacatgaaatctttgatctcaatctgtatatatatatattgtacatgaccgctagtaggtatgaaatgcatttcagagttgaaaactcatgcaacataatcatgtgtttgcataaggaatgttaaaattctttctactacaatttattaacaggggaaaagattcaagcagtgccatctagaccttttctcttctcatttcttcaaaagcagtgctaagtaaaataataataataaaaatggtttatgaaccaaaagactttacattctagcattaggaacacacacaaaaaaatctgtcagcttacaaaagcacaacacataaagaaagagcagtggaatgcgtaatggggaaatgaatcatgtttaaagcaagacttgcattaaattggcaactaagataatttccattttctataggtaaccgtgttccagtttaatgattaccttcatactcaacaagaaaaatggcaaagcaacatcatcaaaataggtcttacaacatatctacatgtatatatgtgtatatatagattcatataaacatacatacatatatatgcattcatatgttggaaaacaaaaggaataagctaatatgttttatttccttcattaaaccagtatatgagtatttttaatttggcaaacaaaaatcaacatactgaaataagtaatacagggtactgagcatttctctatcagcaaatcaatgcctacagtttttatgaaccattgcacagttcaactcgacaatagtactgtaccccagaacatcagaagttaagtagttgtcacattgcttgtctaattgctctctttttcaaggaaacaaataacattgtccattggtgagtcaaaaacaaacatgtcttaagaaaatgaaaaatgataaaaattcctccaaagaggaagaaaaaaaaaaaactaaaccccaccatgttggttagggcaaaatactaagtattcacaactgtagcaatcatgccatttggctaaatgtacagagtacatgtaatgtgagcaaccataactttcactgaacgtgtgtaaatattaaaacagatttcttaaatatttttaacttctaatttgtattttatggtaaggcaatgtgctaattccgtttatggcttaagttgatgtgttataatatgtaaataattaaacatgctgtaaatgaatagtcaatagatacaaataattgtttagtttttttcttaatttttatttattttttactaagtactattactgcagtgcccccagcagagtaatttaaacacccttttgcataaatggtttgggaatcccagacagaagggtttcaatttgctatcacacacacagtcacacacagtcacacacacacacacacacacactcaaacacacataatcttttgctcacccactctgtctctctgcccgtctatgggaaacactctactatttatgtagtcaatataacaaacaagagatagcctttgaaatggcaattgggaaaagcaaaaatgatcattgataacttaatagacttttttatatgtcagagttcaagaaaccaaagtttgcaacttaaatctcagttttcaaacagtaaaagtttgtatagatggcactacagaaaaataaaaatggttattcctaccagctccagaaatgtgttacacaatatacactagaagactttttttttaataatagtattttgtccttaggtagtattgttcctttcaaccaaagactccagaattattgaaaaacaaaaaaggaaaaaaaaaatgagataatgggggaaaaaaaggacgctgcttcacaccaaaggtttaaaaaaatattttccaaatgcactaggaacattgtaaaaaccagaaaggagtgaacagaatgctaatgaagatactgccctaaagaaatctgtacaaaaaaaaaaaaaaaaagcagcactcttaaaaagctactaggtttttggaaattgaagtaaaatatttctgtagatatattcaaaattctgacatcaaattttaatttgcttaaagaagttttatttgtgatgaagatcaatatgattcaaatgatgaaaaacaaaactgtatgtcctttgagttcccaaaccaaatgtcactgccgtgtcacatcctctaatctgatgcccatgtgtgtgtccttgcgccatttgaagcaagttgacagttccatcagtttgtttttgtttttgtttttggttcaataatttgagttcaaccatcgtcattttaaacataattaatcccaacaaagttgcctatagtttaatttaaacctcacagcatttttaatttattttgcatgggtgtttaaatagttctgttacgaatattgtccctgcacagttctaaattgtccttttataaaaaaaaaaagtgatattctagtttcttcacaatgtagtgattatcagccttactaattaccttgtagcctttcttaatatgcacataatgcacattttccgatggctagaaaaatgcaaaacacagtggatatcattgcatctattttcatgtctttctaaaaacaggactattaaaatctctgggatacatgagatagtaaaaaatgaggattataaatgaatagcacataagaattattttcttgaatttaaacttattgcagccagtttcagcatgtaaatatataataatgttggctagtgtgtaattcttgaactaagaaatataaataaaaataaaaagattgtg

SEQ ID NO: 278 - DNA, 34 bp, homo sapiens
gcctggatgtcaacaagatggacccccatgagaa

SEQ ID NO: 279 - DNA, 221 bp, homo sapiens
gagtacaccatgacggtgttcctgcaccagagctggcgggacagcaggctctcctacaaccacaccaacgagaccctgggtctggacagccgcttcgtggacaagctgtggctgcccgacaccttcatcgtgaacgccaagtcggcctggttccacgacgtgacggtggagaacaagctcatccggctgcagcccgacggcgtgatcctgtacagcatccg

SEQ ID NO: 280 - DNA, 449 bp, homo sapiens
taataatggccgtaagcttaaaatagatccagggaggagctcattaacgtgaacatagaaagcagttccgcacctctggccttactcctcttggaaattgctttggtccatttttacttccttttattcgacgcaccagaaaataagacttttaccaacatttttactgcatttgacgatgaactaatttagaccggctaaaataattgttccactgggacacaggaattcaacctcagttcagaaaatccctgacatctgacgtaggaggatttataggtttagtggaaattgctttctcctgctctccagattgcatcctgtgggttgattttttttttgcatgagtaaacatccttctaataatgaacagaccaataatgtcttaagagagaaaaagaacaatcttttcctttttgctgtttctggagagagctgtttgaatttgg

SEQ ID NO: 281 - DNA, 181 bp, homo sapiens
attacccattttttagtgaagctccaaactttgctgatcaaactggtgtatttgaatattttacaaatattgaagaccctggcgagcatcacttcacgctacgccaagttctcaaccagagacccattacatgggctgccgatgcatccaacacaatcagtattataggagactacaactg

SEQ ID NO: 282 - DNA, 183 bp, homo sapiens
ggggagagcggtggcggcggctgcgccgggctgtgagtctctcgccgccggaggaagatgaggctgaagattgggttcatcttacgcagtttgctggtggtgggaagcttcctggggctagtggtcctctggtcttccctgaccccgcggccggacgacccaagcccgctgagcaggatgagg

SEQ ID NO: 283 - DNA, 101 bp, homo sapiens
aaacaagcaattcaagccagcattaaagagtttggatttaacatggtggcaagtgacatgatctcactggaccgcagcgtcaatgacttacgccaagaaga

SEQ ID NO: 284 - DNA, 80 bp, homo sapiens
gttttgatataccttgatgcccactgtgaggtggcagttaactggtatgcaccacttgtagctcccatatctaaggacag

SEQ ID NO: 285 - DNA, 219 bp, homo sapiens
aattatgttagagttgtggaggtttggtgggatgaatataaagactacttctatgctagtcgtcctgaatcgcaggcattaccatatggggatatatcggagctgaaaaaatttcgagaagatcacaactgcaaaagttttaagtggttcatggaagaaatagcttatgatatcacctcacactaccctttgccacccaaaaatgttgactggggagaa

SEQ ID NO: 286 - DNA, 99 bp, homo sapiens
atcagaggcttcgaaactgcttactgcattgatagcatgggaaaaacaaatggaggctttgttgaactaggaccctgccacaggatgggagggaatcag

SEQ ID NO: 287 - DNA, 144 bp, homo sapiens
aacctgcacagatttactcatattccttcaggaaagtgtttagatcgctcagaggtcctgcatcaagtattcatctccaattgtgactccagtaaaacgactcaaaaatgggaaatgaataacatccatagtgtttagagagaa

SEQ ID NO: 288 - DNA, 360 bp, homo sapiens
gaagacagagatgtcaatgaccccatgcccaaccgaggcggcaatggactagctcctggggaggacagattcaaacctgtggtaccatggcctcatgttgaaggagtagaagtggacttagagtctattagaagaataaacaaggccaaaaatgaacaagagcaccatgctggaggagattcccagaaagatatcatgcagaggcagtatctcacatttaagcctcagacattcacctaccatgatcctgtgcttcgcccagggatcctcggtaactttgaacccaaagaacctgagcctcctggagtggttggtggccctggagagaaagccaagccattggttttgggaccagaattc

SEQ ID NO: 289 - DNA, 184 bp, homo sapiens
actgtgtggccccgcaacccagcacaaccaggtatctgcttggaacccagccaccataaagcctgctagctaaaaaaaattttacatctctcagttcattcggcacagacccctgcctcattcagctgtgactctgcttggaaaattcatcagttacaaagcagccaatgcaattatctcaagg

SEQ ID NO: 290 - DNA, 113 bp, homo sapiens
ggttcttcagtgggtgagttatatcttgatgatggccattcattccaatacctccaccagaagcaatttttgcacaggaagttttcattctgttccagtgttctgatcaatag

SEQ ID NO: 291 - DNA, 583 bp, homo sapiens
gaacaggcccgagtactaaaggagagatgccaaggtgaaagtacccaacttcaaaatgagatacaaaagctacagaagaccctgaaaaaaaaaaccaagagatatatgtcgcataagctaaagatctaaacaacagagcttttctgtcatcctaacccaaggcataactgaaacaattttagaatttggaacaagtgtcactatatttgataataattagatcttgcatcataacactaaaagtttacaagaacatgcagttcaatgatcaaaatcatgttttttccttaaaaagattgtaaattgtgcaacaaagatgcatttacctctgtaccaacagaggagggatcatgagttgccaccactcagaagtttattcttccagacgaccagtggatactgaggaaagtcttaggtaaaaatcttgggacatatttgggcactggtttggccaagtgtacaatgggtcccaatatcagaaacaaccatcctagcttcctagggaagacagtgtacagttctccattatatcaaggctacaaggtctatgagcaataatgtgatttctggacattgcccatgg

SEQ ID NO: 292 - DNA, 243 bp, homo sapiens
gtaccagtcaaaaagataaaaattttaatctgccccgactctgtatccggaagttcttcccaaagaaaaaatgttttgtcttcgatctgcccattcaccgcaggaagcttgcccagcttgagaaactacaagatgaagagctggaccctgaatttgtgcaacaagtagcagacttctgttcctacatctttagcaattccaaaactaaaactctttcaggaggcatcaaggtcaatgggcctc

SEQ ID NO: 293 - DNA, 605 bp, homo sapiens
ggcgggactgaagagctggaagaaagcagtcagtaccctcccaacggcccccctcgaaggtctccactctcctctgggctcctccttgcctaatgcagggggtcaccgctggagaagaaccaccactgtcctcgatgtgtcccaagcctggagcgaatccgtcctcttggctctcccagccctatcacaggaatcattcctgggtttctgtccctctgaggctcaccaggtgtagttggccttgttctttgggggtattagcccttctactttctttctcacccaccctgtaccctcttctgtgtgtctctgctatccccctttcctcccaccacccatgtgcatgagcaaatgtgcaacaaaaccctgggactttgcagtcaaatgaagctgagcttcccacatcccgttcttcggtttgccatgagacgatgtggggtttccactgtgtgtctgtcatcacctctttgtctttttttcctaaccctgatctcatacttgtatagagtaataacaacagttgtacttccaccaaaggcatgtggcatatttaccaatgtcatgtattctgaacaaaggcaaaaaatacaaattcctaccattaa

SEQ ID NO: 294 - DNA, 52 bp, homo sapiens
atgatgatcatgctgggagccatctgtgccatcatcgtggtagttattgtaa

SEQ ID NO: 295 - DNA, 34 bp, homo sapiens
gacagagtgcccagcgggtcacatgcatcggacg

SEQ ID NO: 296 - DNA, 152 bp, homo sapiens
ccaaacccatgcgtatcaaagaggatattctcgcctgctcagctgctgagctcaactacggtctggcccagtttgtgagagaaatcactcggcccaatggtgaacgatatgaacctgacagtatctactatttgtgtcttggcatccagcag

SEQ ID NO: 297 - DNA, 59 bp, homo sapiens
ggcccacggacctcagcatgaaaggcggggcctctaccacctccaccacccccacgccc

SEQ ID NO: 298 - DNA, 34 bp, homo sapiens
ctgagagccacagtcaggtctgtcctcaggggtc

SEQ ID NO: 299 - DNA, 89 bp, homo sapiens
gaataatcggtggagatggattgtgaaatggaggtgcataaatatccagtttgccaaccgagctaagaaatgctggataaaagtaaaag

SEQ ID NO: 300 - DNA, 34 bp, homo sapiens
agcttggagcaaagaagatgatgtctcactctgt

SEQ ID NO: 301 - DNA, 80 bp, homo sapiens
cctttcagacggaaggaaagctctacctgatcctggacttcctgcggggaggggacctcttcacccggctctccaaagag

SEQ ID NO: 302 - DNA, 162 bp, homo sapiens
gttgttcatcgagacctgaagccgagtaacatcctgtacagggatgagtcggggagcccagaatccatccgagtctgcgacttcggctttgccaagcagctgcgcgcggggaacgggctgctcatgacaccctgctacacggccaatttcgtggccccggag

SEQ ID NO: 303 - DNA, 359 bp, homo sapiens
ggtggccctttgggctccctgtccaggatttgcgctctggagggtagggcttgctgggctggggactggaggggaacgtggagctccttctgcctcctttcctgccccatgacagcaggcagatcccaggagagaagagctcaggagatgggaagaggatctgtccaggggttagacctcaagggtgacttggagttctttacggcacccatgctttctttgaggagttttgtgtttgtgggtgtggggtcggggctcacctcctcccacatccctgcccagaggtgggcagagtgggggcagtgccttgctccccctgctcactctctgctgacctccggctccctgtgctgccccag

SEQ ID NO: 304 - DNA, 124 bp, homo sapiens
aacacgcccaactacttcgacacgtcgcagcccaggatgcgggagacggccttcgaggaggacgtgcagctgccgcgggcctatatggagaacaaggccttctccatggatgaacacaatgcag

SEQ ID NO: 305 - DNA, 138 bp, homo sapiens
ccccgcgctgtgtactgtaaggacgtgctggacatcgagcagttctccactgtgaagggcgtcaatctggaccacacagacgacgacttctactccaagttctccacgggctctgtgtccatcccatggcaaaacgag

SEQ ID NO: 306 - DNA, 208 bp, homo sapiens
tctactgtctcagtttacattttgtatatttgtatttaaatgaaagtgagactttgagggtgtatattttctgtgcagccactgttaagccatgtgttccaaggcattttagcggggagggggttatcaaaaaaaaaaaaatgtgactcaagacttccagagcctcaaatgagaaaatgtctttattaaatgtagaaagtgatccata

SEQ ID NO: 307 - DNA, 78 bp, homo sapiens
gaagtttgtgtgtatgttacagcccgaagtgtgcctcttggggacaaatcacatattcagtgttttacattcataaag

SEQ ID NO: 308 - DNA, 62 bp, homo sapiens
atctggataatacagagaaaaatgaaaaactgaaattcagtatcattgtgcggcttcctccg

SEQ ID NO: 309 - DNA, 94 bp, homo sapiens
gcctttgggctgcccatcaccgtggcccatgtggatggccaaacccacatgttatttggctctgaccggatggagctgctggcgcacctgctgg

SEQ ID NO: 310 - DNA, 34 bp, homo sapiens
aggagggggtcctggaggctctggcccacgtgga

SEQ ID NO: 311 - DNA, 173 bp, homo sapiens
agtgtctctatgtattcccctgccagtggaactaccgtcccgatcactgcatgtacggaagcaactgcagagaggctgagcatgaaggtgtgtctgttctgcatggaaaccgaggcgtctaccatgacgataagcaaccaacgttcagagcactctatgaagcaatacgggat

SEQ ID NO: 312 - DNA, 260 bp, homo sapiens
tttccctttcaagacaatctctttcaatccatgtattacccccttcagctgaagtttttggagactgtgcacactttatgtggacgaatcccgcaagtttttctgaagcaaattgagaaaacaatgaaaagggcttatgagaaacacgtcatcatccatgttggccccaaccagatgcactgaatattttgtcttgttgcaagtcaattaggtgtcttgtgaccaaggaaatactaatctctaagctgcctgggtctttt

SEQ ID NO: 313 - DNA, 135 bp, homo sapiens
tgtgtgaatatttaatggtgctccatgactgttgagttttaaaaacctcgttaaattttgccaaatcagttgcccccaaaagggaatatgcttttccttatttttttttctaaaatgctatttatctctaaggaa

SEQ ID NO: 314 - DNA, 1558 bp, homo sapiens
gtttgagccgtcgtgcttcaccggtctacctcgctagcatgtcgggccgcggcaagactggcggcaaggcccgcgccaaggccaagtcgcgctcgtcgcgcgccggcctccagttcccagtgggccgtgtacaccggctgctgcggaagggccactacgccgagcgcgttggcgccggcgcgccagtgtacctggcggcagtgctggagtacctcaccgctgagatcctggagctggcgggcaatgcggcccgcgacaacaagaagacgcgaatcatcccccgccacctgcagctggccatccgcaacgacgaggagctcaacaagctgctgggcggcgtgacgatcgcccagggaggcgtcctgcccaacatccaggccgtgctgctgcccaagaagaccagcgccaccgtggggccgaaggcgccctcgggcggcaagaaggccacccaggcctcccaggagtactaagagggcccgcgccgcggccggccgccaggcctccccatgccaccacaaaggcccttttaagggccaccaccgccctcatggaaagagctgagccgcttcagactgcggggcaagcgggccgcggctcccttcccctcccctcccctcgcccgccttcgccgcccggcctcgagtccccgcccgcccccgctcccgtcccgcaccgcctgccgcgtcggcctcgggccctgccctgtccgccgtccgccctccggtagggttcgggccttccggatgcggcttgggcgctcttcggggacctccgtggcgcggaagacccgagcctgccggggggaggccggcggcgccgcacctgcccgcctcggcgttcgtgactcagccgccccatcccgagtcgctaaggggctgcggggaggccgcagcaccttctggaagacttggccttccgctctgacgcagggccgaggtgggcagtccaggccgagaggccggcggccctgaaggtgagtgaggccctcggcagctgcagccggggtgtctggtacccccccggcgtggtgcttagcccaggactttcagacgcggccgctggccgggaggctttggtgggagagacgcgatcgccgatttcggtctggcgccccttctgcggccgggacccaggcctttcacatcagctctccctccatcttcattcataggtctgcgctggggccgggacgaagcacttggtaacaggcacatcttcctcccgagtgactgcctcctaggaggacatttaggggagggcagaggcctgcagtttggcttcacggctggctatgtggacagcaagagtcgttttcgcggaagccgactggcagccaggcctgtcgggccccccgacgccgccccatttcccttccagcaaactcaactcggcaatccaagcacctagataccagcacaagtcggttaatccctgtctggactgagcctccgttggcttctgaactggaattctgcagctaacccttccacgactagaaccttaggcattggggagttttagatggactaattttattaaaggattgttttttttttaa

SEQ ID NO: 315 - DNA, 333 bp, homo sapiens
ttggagaacatcagcttgccagcagctctgaggtgcaaaatggaagtcccatgtctcagactgagactgtgtctaggtcagtcgcacccatgagaggtggagagatcactgcacactggctcttgaccaacagcacaacatctgcagatgtgacaggaagctctgcttcatatcctgaaggtgtgaatgcttcagtgttgacccagttctcagactctactgtacagtctggaggaagtcacacagcattgggagataggagttattcagagtcttcatctacatcttcctcggaaagcttgaattcatcagcaccacgtggagaacgttcga

SEQ ID NO: 316 - DNA, 34 bp, homo sapiens
acagttcacgcctctagggagtccaacgcggtgg

SEQ ID NO: 317 - DNA, 175 bp, homo sapiens
aaagaataaaaatgacataagcaaactcatcttcaaaagtggagatttccaaatgtccccgtatgctgaataccccaaaaatcctcgctcacaagaatggggccgagaagctattgaaatgcatgagaatggaagtaccaaaaacctcctccagatgacggatgtgtactactcg

SEQ ID NO: 318 - DNA, 34 bp, homo sapiens
cggatgtgtactactcgcctacaagtgtaaggaa

SEQ ID NO: 319 - DNA, 199 bp, homo sapiens
atttttagaacaaggaatctgtaggtatggtgcccagtgtacttcagcacattcccaggaagaactagcagaatggcagaaaagatatgcttcacggctgataaaattgaaacagcaaaatgagaataaacagctctcaggcagttacatggaaaccttgatagaaaagtggatgaattcattgtctcctgagaaagtg

SEQ ID NO: 320 - DNA, 334 bp, homo sapiens
gttcaaccttaaagtgcaattgcagattctggcaagcttcatgctcactggtgtttctggaggtgcaaagtatgctcagaatggacaactttttggtcgctttaagcttactgaaacactttctgaagatactttggctggacgactggtgatgaccaaagtcaatgctgtttatttattaccagtccctaaacagaagttagtacagacccagggaaccaaagagaaggtttatgaagctactattgaagaaaaaacaaaggaatatatatttttaaggctatctagggaatgctgtgaagaacttaatcttcggcctgactgtgacacacag

SEQ ID NO: 321 - DNA, 200 bp, homo sapiens
tttgggaacggtttattgccctgtgtcatgaaaacagtagcctacatggaatcacttttgaacagatcaaagcccagttagaggctttagaactaaagaagacatatgtgttgaatccgctggcacctgaatttatcccccgggctctaagactgcagcattcaggaagtaccaacaaacagcagcaatcaccacccaag

SEQ ID NO: 322 - DNA, 114 bp, homo sapiens
ctcagctttctcctgcatatcaggcgggacccaacaatgctttttttaatagtgcagttgctcatcggccacagtctcctcctgcagaagctgtaattccggagcagcagcccc

SEQ ID NO: 323 - DNA, 205 bp, homo sapiens
gaaattttgggaacggtttattgccctgtgtcatgaaaacagtagcctacatggaatcacttttgaacagatcaaagcccagttagaggctttagaactaaagaagacatatgtgttgaatccgctggcacctgaatttatcccccgggctctaagactgcagcattcaggaagtaccaacaaacagcagcaatcaccacccaag

SEQ ID NO: 324 - DNA, 1332 bp, homo sapiens
acctcacgagtggaaatgtggtattctcttttttttctctcccttttttgtttggttcaaggcagctcggtaactgacatcagcaacttttgaaaacttcacacttgttaccatttagaagtttcctggaaaatatatggaccgtaccatccagcagtgcatcagtatgtctgaattggggaagtaaaatgccctgactgaattctcttgagactagatgggacatacatatatagagagagagtgagagagtcgtgtttcgtaagtgcctgagcttaggaagttttcttctggatatataacattgcacaagggaagacgagtgtggaggataggttaagaaaggaaagggacagaagtcttgcaataggctgcagacattttaataccatgccagagaagagtattctgctgaaaccaacaggttttactggtcaaaatgactgctgaaaataattttcaagttgaaagatctagttttatcttagtttgccttctttgtacagacatgccaagaggtgacatttagcagtgcattggtataagcaattatttcatcagttctcagattaacaagcatttctgctctgcctgcaggcccccaggcacttttttttttggatggctcaaaatatggtgctgctttatataaaccttacatttatatagtgcacctatgagcagttgcctaccatgtgtccaccagaggctatttaattcatgccaacttgaaaactctccagtttgtaggagtttggtttaatttattcagtttcattaggactatttttatatatttatcctcttcattttctcctaatgatgcaacatctattcttgtcaccctttgggagaagttacatttctggaggtgatgaagcaaggagggagcactaggaagagaaaagctacaatttttaaagctctttgtcaagttagtgattgcatttgatcccaaaacaagatgaatgtatgcaatgggatgtacataagttatttttgcccatgcctaaactagtgctatgtaatggggttgtggttttgtttttttcgatttcgtttaatgacaaaataatctcttaatatgctgaaatcaagcacgtgagagtttttgtttaaaagataagagacacagcatgtattatgcacttcatttctctactgtgtggagaaagcaataaacattatgagaatgttaaacgttatgcaaaattatacttttaaatatttgttttgaaattactgtacctagtcttttttgcattactttgtaacctttttctatgcaagagtctttacataccactaattaaatgaagtcctttttgacta

SEQ ID NO: 325 - DNA, 100 bp, homo sapiens
gactatgatgccaacatcgtagctgtggtgaatgacacagtgggcaccatgatgacctgtggctatgacgaccagcactgtgaagtcggcctgatcatcg

SEQ ID NO: 326 - DNA, 157 bp, homo sapiens
agaacagcccatcttcaccacccgagcgcatgtcttccagattgaccccaacaccaagaagaactggatgcctgcgagcaagcaggcggtcaccgtttcctacttctatgatgtcacaaggaacagctatcggatcatcagtgtggacggagccaag

SEQ ID NO: 327 - DNA, 187 bp, homo sapiens
aaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacag

SEQ ID NO: 328 - DNA, 120 bp, homo sapiens
gaggctcgagtccccataatcagcaatgatgtctgcaatggcgctgacttctatggaaaccagatcaagcccaagatgttctgtgctggctaccccgagggtggcattgatgcctgccag

SEQ ID NO: 329 - DNA, 143 bp, homo sapiens
gccaacaggccggggtactccaggaggctcgagtccccataatcagcaatgatgtctgcaatggcgctgacttctatggaaaccagatcaagcccaagatgttctgtgctggctaccccgagggtggcattgatgcctgccag

SEQ ID NO: 330 - DNA, 337 bp, homo sapiens
ttcaattttctttagttttaattgaaaatgtttgcttttaaaactgataggtattgttggaaagcaggatgaagcctgagccagtggaaaagcttgttacagaaaaaacattttgtgttattgctgtggtgtgcatgatttgcaaagattaagtgcattttctctgtctatactgattattgtatatagaggatgttataaatatacatatacatttttgccattatgtaaatcccatgatttcaactgtaaacatctgtccattggtgtagctttacaaaccattcactgattttgtgtaatttaacaatagatatgaaataaagtttaaattaca

SEQ ID NO: 331 - DNA, 3316 bp, homo sapiens
gcagaggacgaagaggaggaggatgaggaggacacagaagatggcgtcagcttccagccctacattgaaccaccttctttcctggggcaagagcaccaggctccagggcactcggaggctggtggggtggactcagggaggcccagggctcctctggtcccaagcgaaggctcctctgcttgggattcttcagacagaagctgggccagcactgtggactcctcctgggacagggctgggtcctctggctatttggctgagaaggggccaggccaagggccgggtggggatgggcaccaagaatctctcccaccacctgaattctccaaggactcgggtttcctggaagagctcccagaagataacctctcctcctgggccacctggggcaccttaccaccggagccgaatctggtccctgggggacccccagtttctcttcagacactgaccttctgctgggaaagcagccctgaggaggaagaggaggcgagggaatcagaaattgaggacagcgatgcgggcagctggggggctgagagcacccagaggaccgaggacaggggccggacattggggcattacatggccaggtgagctgtcccccgacatcccaccgaatctgatgctgctgctgcctttgcaaggactactgggcttcccaagaaactcaagagcctccgtacctcccctgggcggcggaggggcattgcacttccgggaagtccacctagcggctgtttgcctgtcgggctgagcaacaagatgcccctccctcctgtgacccgccctctttaggctgagctataagaggggtggacacagggtgggctgaggtcagaggttggtggggtgtcatcacccccattgtccctagggtgacaggccagggggaaaaattatccccggacaacatgaaacaggtgaggtcaggtcactgcggacatcaagggcggacaccaccaaggggccctctggaacttgagaccactggaggcacacctgctatacctcatgcctttcccagcagccactgaactcccccatcccagggctcagcctcctgattcatgggtcccctagttaggcccagataaaaatccagttggctgagggttttggatgggaagggaagggtggctgtcctcaaatcctggtctttggagtcatggcactgtacggttttagtgtcagacagaccggggttcaaatcccagctctgctgttcactggttgtatgatcttggggaagacatcttccttctctgcctcggcttcctcatctgcagctacgcctgggtgtggtgagggttctaggggatctcagatgtgtgtagcacggagcctgctgtgtcctgggtgctctctacgtggtggccggtagaattctccatctatccaggctccaggagacccctgggcatctcccacctgtggcccctaaacccagagtgactgagagcacttaacattcagcttgtctcatccccagtctacctccttccttctaccctcactgcctcccagtcaggagagtgagctctcagaagccagagccccacccaaggggaccctggtctctccgccttcacctagcaatgggaaccctgcttcccaggggaggaaccaactgctccaccttctagggacccagtttgttggagtaggacagtaacatggcaggaatcggacttctgggcctgtaatcccagtttggatggcacgttagactcttggttgaccgttgtggtccttagaagtcccattctcccttccagttatgagaaaccaatgccttctagattcaggtgactatccttacctgggggtgctgatgcatcctcagttaacctacacccacctgaatatagatgagcgtagctgagttttcacccgtaggaccgaagtgttttgtggtggagtatctgaacaaccttggctctgtggccattcaacctgccaggactaacatttctggatttgtgaagaagggatcttcaaagccattgaacccacagagctgtgttgctttaaagccaccacaagggtacagcattaaatggcagaactggaaaagcttcttagggcatctcatccagggattctcaaaccatgtcccccagaggccttgggctgcagttgcagggggcgccatggggctataggagcctcccactttcaccagagcagcctcactgtgccctgattcacacactgtggctttccacgtgaggttttgtttagagggatccactactcaagaaaaagttagcaaaccactccttttgttgcaaaggagctgaggtcaagggtggcaaaggcacttgtccaaggtcgcccagcagtgctgctctgatgacttgtgcacatccccaagggtaagagcttcgatctctgcacagccgggccaacctctgaccccttgtccatgtcagtaaaatatgaaggtcacagccaggatttctaagggtcaggaggccttcaccgctgctggggcacacacacacacatgcatacacacatacgacacacacctgtgtctccccaggggttttccctgcagtgaggcttgtccagatgattgagcccaggagaggaagaacaaacaaactacggagctggggagggctgtggcttggggccagctcccagggaaattcccagacctgtaccgatgttctctctggcaccagccgagctgcttcgtggaggtaacttcaaaaaagtaaaagctatcatcagcatcatcttagacttgtatgaaataaccactccgtttctattcttaaaccttaccatttttgttttgttttgtttttttgagtcggagttttgttctttttgcctaggctggagtgcagtggtacaatctcggctcactgcaacctccacctcccgggttcaagtgattctcctgcctcagcctcccaagtagctgggattacaggcacccgccaccacacctggctaatttttttgtatttttagtagagacggggtttcaccatgttggccaggctggtctcgaactcctgacctcaggtgatccgcccgcctcggcctcccaaagtgctgggattacaggcgtgagccaccgcgcccagccaaaccttactatttttttaaagaattttttccagagtttaatttctgacatagcttaagttttccagtaactctaaactccatctcctttatcgtcattaagtcattcacaaaaagccaggagaagcatttggaaagggcatgataatcagtataataatttgccttgtgtggtcagcacttaactgtttacaaagccctttcacgtgca

SEQ ID NO: 332 - DNA, 75 bp, homo sapiens
gcatcaacttctaactcatctcgccaggagaggcctccagtgaaacccttcattctcactcggaacatggctcaa

SEQ ID NO: 333 - DNA, 107 bp, homo sapiens
ggcccatgccgtagacatctggactcagtgctgcagcaactccagactgaggtctaccgaggggctcaaacactctacgtgcccaattgtgaccatcgaggcttcta

SEQ ID NO: 334 - DNA, 120 bp, homo sapiens
ggcccatgccgtagacatctggactcagtgctgcagcaactccagactgaggtctaccgaggggctcaaacactctacgtgcccaattgtgaccatcgaggcttctaccggaagcggcag

SEQ ID NO: 335 - DNA, 536 bp, homo sapiens
tgatgatactgatttatttaatggatctcaaaacaaacttttaacttaaggcacttttattttgccaacaaataacaataaacaaacattgaaacggttcactataaaataacaaatggctaatgtacctgaatttttcagtaaaaaaatgaacttctaataccagttgcctagtgtccacctcctatcaatgttacaagcatggcactcagaacagagacaatggaaaatattaaatctgcaatctttatgatgtaaatttaccatcctgatgtataaatattttgtggtttataaatttttttgctaaaacctacagaaaataagcactgaactgtcttatgattcaccatttgttcacactgaaaattttcattgatggggaatacaaaaatgttttaataactgcatttaaaaataaatgcaggctcacttttccacatgtaaaaagcaatgtaatacctgcacatcataaaatgtgaaaagggggttatttttgtccaaatacacccattaaaaaggttttaaaaatacaa

SEQ ID NO: 336 - DNA, 67 bp, homo sapiens
cccgacattgtcacctcctctttgaggggttagaagaagctgagatctcccgacagagctggaaatg

SEQ ID NO: 337 - DNA, 34 bp, homo sapiens
cctccgtttatcttcagcccgacattgtcacctc

SEQ ID NO: 338 - DNA, 49 bp, homo sapiens
cattgcactttgactatggaaacagaggctattgatggctatataacgt

SEQ ID NO: 339 - DNA, 168 bp, homo sapiens
gtgaacgccccttccactgtaaccagtgtggagcttcttttactcagaagggcaaccttctgagacacataaagttacactctggagagaagccgttcaaatgtcctttctgtagctacgcctgtagaagaagggacgccctcacaggacacctcaggacccattctg

SEQ ID NO: 340 - DNA, 144 bp, homo sapiens
tacctcctatggaagattgtaaggaacaagagcctattatggacaacaatatttctctggtgccttttgagagacctgctgtcatagagaagctcacggggaatatgggaaaacgtaaaagctccactccacaaaagtttgtgg

SEQ ID NO: 341 - DNA, 78 bp, homo sapiens
gtaaactgaaatgtgacgtctgtggcatggtttgcattgggcccaatgtgcttatggtacataaaaggagtcacactg

SEQ ID NO: 342 - DNA, 34 bp, homo sapiens
taaaaggagtcacactggtgaacgccccttccac

SEQ ID NO: 343 - DNA, 351 bp, homo sapiens
gtggaggaagagaagggcgtgctcgtgtcccagaattacacgctgccgtgtctcagccctgacgcccagccgccaaacaacatccacagcccagccatccgggcatatctcaagacaatcagacagctagacaacaaatctgttattgatgagatcatagagcacctcgacaaactcatatttcaagatgcaccagaaacaaacatttctgtgccaacagacacccatgaatgtaaacgcttcatcctgactatttctcaacagttttcagagtgcatggacctcgcactaaaatcattgacctctggagcccaacaggccaccacttaaggccatctcttcctttcggat

SEQ ID NO: 344 - DNA, 170 bp, homo sapiens
gttttctcacgatgagtcggaggaggatatcgtgtaaagatctgggccatgctgactgccaagggtggctgtataagaaaaaggaaaagggaagtttcctaagcaacaaatggaaaaagttctgggtgatactgaaggggtcgtcactgtactggtatagcaatcaaatg

SEQ ID NO: 345 - DNA, 86 bp, homo sapiens
aatgttacagtgaaagtgaacaggaagatccagaaatagctgcggagacaccaccccctcctcacgcttcccagactcagtctttg

SEQ ID NO: 346 - DNA, 194 bp, homo sapiens
aagagacaaaagtgtctgaagatgatgaaatggagaagctgtacaaatcattagagcaagctagtctatctcctcttggggaccgacgaccttcgactaaaaaggagttgagaaaatcctttgttaagcggtgtaaaaatccatctataaacgagaaactccacaaaatccgaacattgaatagcacattaaag

SEQ ID NO: 347 - DNA, 238 bp, homo sapiens
tgtaaagaacatgatctggccatgattaaccagttgctggatgacccgaagctgacagccaggaaatacagagagtggaaagtcatgaacaccctgctgatccaggacatctatcagcagcagcgggcttcgcctgcccctgatgacactgatgacaccccccaggaactcaagaaatcaccttcttctccctctgttgaaaattccatttgagacaaagtcagggttttctcctctt

SEQ ID NO: 348 - DNA, 5320 bp, homo sapiens
atattttatcacaagcaactcttcaagatgttgcaaaagcttacatttttccttaaaaggaaaactgaaacccagtccttcaagcatcagcttcccatctaaagatgcacgttagatgaagataatcaccgaatacaccagggctcctcatttcagtcgtaacgcatccattgagattggaatgattgagttcagttgaaaggcgttttttgtgttcctactgggagctcacggtgatgtggggctctaggggggatgtgagaaatgtctcttctgttcattgctctccagagaatgctatggacaagaatcaaattttaccaggagtaattgcccatggaaacattctgtttttcaaaagaagaatgtacatattccacttgactgggaagctgtgtggcccaaatcactgtttgagtggaggaatgttttctagagcagtgactggaaggacaaacgtggtgttgcagaacaaaaattccttgcctttggtatggaatggtggtgccttttccatgtgatgagaaatgcttcatagacagtggcatgtgggagagctgaaggaggaggcgtgttccatattccactcattcaaaggacagcttggtgttttcacactttatgcaggttaggggatctgattaatgtttcctttccttacaacagctagagaaaacatctcatcacaattggagacctctacttcctttgagctttcctactcaatggtaggagataggaatatgttccgcaaagaatgtctactgctcttgtccttggctgtgatccctatacatagtgtgaatttatatccaggtttcccaggatcaatattgacaagacgactttggcatctgaattgttttaatatctttactcaaaaaaacaaaaaaacaaaaacccatggaagcaacaaaagagttaaacatgagggatttatttttgtatgtttatatgtcacttgtgtgctatgtgaatagctatgtttgttatttaaatatatttatagaagttcaaattactagtgtttaaaagatgatatactattatatattgtgcttagtatatatttgatgagtctatactttttgcttgagaaaaatagaaatactattgattcactggtgttatgatgttgcactgtgtctgaggaatgctcaggaaatcttgtgggaaaaaaatggggcatgcattcataatttttcattttcttccccaccccaacccccatgagctcttaaatttagatttctatctcttgtaagtcactcttttaaccagcaaaggactttcatcttagattatcctttctattaaataaactttaagctttgtgccatttatgcctgcaaaccttagaccaaaggcaacacaactaatgtcatcttttcattaaagactaatagaaatttagaaatcattgtgttaaagagagctatagagaaacaatatgaaagtcttcccttactaatcaccatgaatatttctcaccataggaataaccacaacccaccaagtacccatgttggccctgcctacccgtaacaaattgttcacacaacctattttgttttatagcttcaaagtgtggcttgacattcaaatggaaggaaccacctgaattatatctgcaaacaaaatggaaatagttgttgaatagttccccaagctttactaagaagccttaatactctgtaaacaaacaaacaaacaaaaaacaaccttagcattaagaaataaaaacaaaaaacaagaagcaagttgtactgctagaaacttctctaagataagaggccaggccagtcagtttggcccagcaaacatgtgggtacttctccacctagggaagccactgaagatgagtttgacctgtggtgacctgacagagagaacattcagatcctcagattgcaccctgatgccctggtttaggaaagcagtgctttaaccttttgcccctggaaattactgattctttctgtgattagcagataccagaagggaaattatttaggattggcaatgtaaagagtattgaaggaacaacaaataacaatagcagaaataacagtgtgacataaagtgcccttccacctatttttctttggtttgcaacaccagcctgtagacttccagtttttcttaaattgaagagctccagacacttttgagcagggtttgtgaatagctttgaatgctttcctttggcttttcgtgaagacatatttccccagcctttcaactgtaaactcctttagtaccattgaaacttaaggcaaaaaagatccctgggattacttagccaaggcttaaatttatttttcaaagtcaagaacattagcctagaagtttttgctttgctagttaaagcttactttgcagagcttcccaaagtgaaagggaaactgaacttcacaattttgcacctggctttctggagccccattgcctgctcattcgtgtttttccccctgtaaattattttgcttttgaccaggttgtgctagaaagtagggctgcttctgtgagttctcagcctaggctgtggccactaagacgtgggtggggggatagacaggagtgagagggagagtgggtgacactttccgtgacacttaatgtaggaagacatgggcttctctctcttggcctcaagccacttttctggtccaaagaatattgggaaaactggattactttcagtatatttcttgagaccgggtcatctctgaaatgcatttggggtagtttcctggggtccaagggccaattcctatacccagtcctgcagtgtctgtgagctgtggaatgagagagaaaaccatggctgctcaggaaggaagtggagaatctctcagcacacgtagatccaaatctgtggcacctgctaaagattaggatttggagaggactcctctttggggttgtgaagaggaccttttccctctgttgttagcctgtagatccagaacggagcctgatgtgatgccttcactggttgatatattgcaaatagacactctgagccctgcattgcaagggcgtcttttgctttaaagaattcttagttttgccacaacagctcaaatctcagaataggatcaaaggcctagaaagactcctaaagtataggattacaaagactccaggaagcgtaagaaagagattgtgtttggagtcagatttcaatgtgaaacccatgccttacatttacacagcactttgtagtttgcaaattggtgtgttttcagccccctcccgctgccccatgctctgagatgattgtggattattacagtgaagtgcctggcccggagcccccagtgatgaagcaggaacttgagtctcaggactcccagcggctctctgcccatttcctcctctccgccccccacctcgcttcacctgttaccttgtgaagttgctgttggcacatagcccactaggaaaccaagaagtttgactgggagcgtgtgtttcattgcactgggcttagcatacggctggccctggtggctgtttggctaaattctccatttgaaacctaaagggccctctctgtgcttgagcacagtctcctcctagtggtcctgcctcaacctgctggcgggctgtctatggattggggtttcacctaatcatgctgtgtttcctttcttttctttctgtacattagactacttgggaggtgctgtgtgatccttggatgaaaaacatatatttaaagaaatggagagtgtttattttgtttgctttttgaggcatgtgctgtcaaatgttagagaccaaacttagtgattagaggttggtcctttgaccttcttctcttaattgcttttcatgtaatcatgataatttactcaggtagcactccagtcacaaacaatgtcacagagatgaaacagattcaaagaaaaaatatttttaagtcttttgaaaagaagtgctattcaaatttaagacatgtttaaattcataggtacattatctcttagtaagaaatacttttttttttctttttttagacagagtcttgctctgttgcccaggctggagtgcagtggcacgattttagctcactgcaacctctgcctccctggttgaagtgattctcctgcctcagcctcccgagtagctgggactacgcccggctaatttttatatttttagtagagaccggggtttaccatgttgcccaggctggtttcaaactcctgacctcaggtgatctgcctgcctctgcctcccagagtgctgggattacaggtgtgagccaccacacctggccgaaagacagtattttgatatagccacatacatgatttaatgagtaatgttaagtacagaaaagctgccatatccacatagttttagggggcatttcttcttcagtctaacaagaatatgatatccaattgtggttttccaatttatttctcttccaatttcttttgtatacctcacaggagaaattgaatcctagaactgggagatgactgccctgcccagggttttgtaatacataattgaaaataaaagtccctgaaactaaatgtttgcagcctgtatgaaatggactgtggaagggggataatttctctcctcacagaaagccactcgtctccttgaatgaggcattgagcagacacacgtttctctgtgtcgtggggcaggaagcaattcccttgcctcttcagcacatctcagttctttcccacatgaccccagaaccacatagaaaacattgaaaataatggattcttcattgtatttgaattttttttttttaacctttagcccaaaagggaagtgtgcctcgtggaaagagcaaatgccgtaaaatagaaaccaggagttttaatcgccatatattttaatgtgcttgcatgatgggaatgtgacttttagcactgcatggtgtttgggggcaagatattaatgtggaaatcttagcttaagttttactgtgctctcatcctgtgtcagctacccctctaaactgaaaaagaggcatggctcatttctctcctgctttatgtttttgaggttgacttgtaagataaaaataaaaataagaaaaaaatatatctaggcaaatgacatgaaaaaaaagttgaaatatactactggtgttcatttttgtcctgcagtgttgctttctcaaagaaataaacatgtggctggaagtgtttaatggctgcgttttgatcgtctacaacaaggttacagtgccctctggtggcagtcatcaaaatcgcttctagacttgtttttatttttaaataaaagatctaattatattggaaatctgtttcaacaa

SEQ ID NO: 349 - DNA, 99 bp, homo sapiens
gagtactcagctgtgaagtccctggaagtgattgtccgggccaacatcacagtgaagtcctccataaagaacttgatgctccgagatgcctccacagtg

SEQ ID NO: 350 - DNA, 141 bp, homo sapiens
tgtggcttttttaaaagaaaatatcaacaactgaacttggagagcatcaggaaggcccagctgaaatcagagaatctgctcgaagaagagaattaggacctgctatccactgggagaggctatcagccagtcctgggactt

SEQ ID NO: 351 - DNA, 147 bp, homo sapiens
agcctcagacgctgcgtggagcggcggagccggagggaagcaaaggaccgtctgcgctgctgtccccgccccgcgcgctctgcgcccctcgtccctggcggtcgctccgaagctcagccctcttgcctgccccggagctgtcccggg

SEQ ID NO: 352 - DNA, 161 bp, homo sapiens
ctgggcacctgccccaccaaggaggacaaggaggcctttgccatcgtgtcagtgcccgtgtctgagatccgagacctggactttgccaatgacgccagctccatgctggccagtgccgtggagaaactcaacgagggcttcatcagccagaatgaccgcag

SEQ ID NO: 353 - DNA, 438 bp, homo sapiens
ccgatccagtttaaatgccaaaatggaagagtgtgttaaactaaactgtgaacaaccttatgtgactacagcaataataagcatcccaacacctccagtaaccacaccagaaggagacgataggccagaatcccctgagtactcaggaggaaatattgtcagagtttctgctttgtaagacaattggaataaggtctaagagaattcgagccctggctgtgaaaagaatctcaacatagaagaaagaagaaacaataaatattctgcagattaatgcagcaaagaaagaaggttggtagtgaaacacaaagcttccaatcttaaggatgtgaataaaaccaccaaatggcatttctagacagtttgacctgttatacagagtaatattctgtggccctttgactttgtgaatgagcacaatgaaatgccgcctactga

SEQ ID NO: 354 - DNA, 1019 bp, homo sapiens
gtgtgaaccccagcgccaactaccacgccctgtaccgctacttcgtggagctctggatctacttggggctggcctggctgtccctttttgtcaactggaaggtgagcatgtttgtggaagtccacaaagccattaagaagcggcggcggcgacggaaggagtcctttgagagctccccacactcccggaaggccctgcaggtgaaggggagcacagcctccaaggacgtcaacatcttcagctttctttccaagaaggaagagacctacaacgacctcatcaagcagatcgggaagaaggccatgaagacaagcgggggtggggagacgggcccgggcccagggctggggcctcaaggcggtgggctcccagcactgcccccttccctggtgcccctggtagtctactccaagaaccgggtgcccaccttggaagaggtgtcacagacactgaggagcaaaggccacgtatcaaggtccccagatgaggaggctgtggcacgggcccctgaagacagctcccctgcccccgaggtgttcatgaaccagctggaccgcatcagcgaggaatgcgagccatgggacgcccaggactaccacccactcatcttccaggacgccagcatcaccttcgtgaacacggaggctggcctctcagacgaggagacctccaagtcctcgctagaggacaacttggcaggggaggagagcccccagcagggggctgaagccaaggcgcccctgaacatgggcgagttcccctcctcctccgagtccaccttcaccagcactgagtctgagctctctgtgccttacgaacagctgatgaatgagtacaacaaggctaacagccccaagggcacatgaggcagggccggctccccaccccacctttgatggcctcttcccccctcaccctagggtgtcccgagatgaccgggacgcctggcccctggtgggggggcagcctcggaactgggagtggggggccaggggccttcctaaccttccatcatcctc

SEQ ID NO: 355 - DNA, 143 bp, homo sapiens
tgggattcttaccccgatgtgctggcaatgcagttcaactgggacggctactataaggaagtgggctctgctttcatcggcagcagccctgagtttgagtttgcactctactccctgtgcttcatcgccaggccaggcaaagt

SEQ ID NO: 356 - DNA, 186 bp, homo sapiens
aaatgcatcttctgcagacaccgggttaagagggtctctggagcctgcatccagtgttcctacggtcgctgcccggcctccttccatgtcacttgtgcccatgctgctggggtactgatggagcctgatgactggccttatgtggtgaacattacatgctttcgacataaggtcaaccccaacgtg

SEQ ID NO: 357 - DNA, 263 bp, homo sapiens
atcccagtgtctgagtgaactaacagtcctgctttcagccaccatttgcacaagacacccagcactgaaagtcccgctgccaggagcaagggatcctttggaagcacccgccctttgtgccttgttgggggaaaccggtgacgcagaagtgagtgtggatacaccagagtttgcattggaaggaatgagtgtcacgtggggagggaaggggccagtggaccttttgtaagctttccactcaataaaatgaacctgtatggcaa

SEQ ID NO: 358 - DNA, 102 bp, homo sapiens
ggggatggctgaagatggaacgatcgttcttcctcaagaagcggcgggcagattttgtggctggctctctgagtggacgggtcatagtggctgggggacttg

SEQ ID NO: 359 - DNA, 72 bp, homo sapiens
accagcggcagaccacaggcagggcagaggcacgtctgggtcccctccctccttcctatcggcgactcccag

SEQ ID NO: 360 - DNA, 248 bp, homo sapiens
tgagtacaccgtgcacctgggcagtgatacgctgggcgacaggagagctcagaggatcaaggcctcgaagtcattccgccaccccggctactccacacagacccatgttaatgacctcatgctcgtgaagctcaatagccaggccaggctgtcatccatggtgaagaaagtcaggctgccctcccgctgcgaaccccctggaaccacctgtactgtctccggctggggcactaccacgagcccagatg

SEQ ID NO: 361 - DNA, 396 bp, homo sapiens
ggtgactcagggggaccgttggtgtgcagaggtaccctgcaaggtctggtgtcctggggaactttcccttgcggccaacccaatgacccaggagtctacactcaagtgtgcaagttcaccaagtggataaatgacaccatgaaaaagcatcgctaacgccacactgagttaattaactgtgtgcttccaacagaaaatgcacaggagtgaggacgccgatgacctatgaagtcaaatttgactttacctttcctcaaagatatatttaaaccaacctcatgccctgttgataaaccaatcaaattggtaaagacctaaaaccaaaacaaataaagaaacacaaaaccctcagtgctggagaagagtcagtgagaccagcactctcaaacactggaa

SEQ ID NO: 362 - DNA, 3243 bp, homo sapiens
atgaaaactgcaaggacaagtactacaactgcaacgtggtggtccaggcaagactctgtgtctacaactactacaagaccgcctgctgtgcctcctgcacccgtgtggccaacaggcagacgggcttcctggggagcagataacactcctgcacccccatcagtagggcagcatcactgccttcccgggggcttcagcagtgcgcctggctggctgctgctccaccacgggccccctggcccaggcgctgccaaccaacttagtcaccacccctgcctccggtgaatgcaccccgtggtacccaggggctttttacacaagatgtttgaaagccacagtcagtcctttaagcatcaccatgtactgatgatcccctccttggacctggcatctgctaatggtgccctttgaaagtcaagcagtgggaagtacatggagctctcagccctgctcccatctggcaccttcaagtcagcagatgggccactgactgagcactgccccgtccctggtgctactggtctttctaaacttagcaccctggagagtccaaggaggcagcgcccccaacccagcgccccactaagccttgctgacacgcgtgcatccctctgtgacctcagcccagatgtgcctgttttcattctcaaagacattagactgttttcctgccctatgacacagatagctcacatgaatattgtgctttatttagcaggtgtactcacagatactagctccttagcagctcacaacatcccagaatgggaggcagggggtgactcattatccccattttactgacagggaaactgaggctcaacttaagtaattgacctgccaggtatattcacccatccagtggaagagctgagtccccgccccagtcatctaccagtatccagcctggggcctgtacttagatgtgaaaggtgctgcttcatttctgaccaagagactgagaagtttcccagaatgcaaacaaagcccaggcccctgaaatctttccggtcaagcctttatcccagcactcagttgttttggatgtctgttcctacttgcccttacccccaaagttacagatcctagttacaggactctgccagctttgttaaactgtccgtgagacaagaaagccattggggaaaccaggtgattgcctgaaattcttactccgttccaagtgctgttcctcccaggaaatcaaaggccagggtccttatggccgtggagccttcccgaccacagagccaacttgtgaagcacacagctctgcagcctgggctctgccctgcctcagccgcctcccccacgctcttcaccacgttcctggagagtccggccaacctgtcccagccgaaacactgctgtattagaaaaagtctctttctggtctttctggttttgtttatgaatttccctctgtggccacaaattcctcccctcccccatgactcacagtccatatggcccacccccagacttgagcaccaagctctgcattaatgcagttggcctgcgacaaggagctgtggacccttccccatctcttccaattcactttccccaactatccagttccagaggccgcaggcctggaaggatgcagtgcatattgaaaggtggaccctctgaaaacagttaagaggaatatatgtatgttttacccattaagaaaaaatggcaagctaaacaaatgttaaacttacagaaaatttgtcttatggtcctgagcatatttcccttttagagcaagcctggattcttagcaaagtgtttcccccatttgctcttttagctgacaaatctgccactgtgatgatggtttgcagcttttggaagcagtatggcaacctggcctgacatgctctttaggcttccactaacctggggctttcagaaattctatttggcctttctgtgggtagctttccagcttctcttctagggagccccaggcatcatttcccaaaagcatccccatctcctgattctcttggaactcctacagataagcatcctggcagaggcccaggctcccaaaccgacaaagtgaaaagagaccagagaggccaagcatattgactggtgctgttcagggcctgctcttttccactcaccacttgttttgctgcttgtcacgaggagagttgttcctgtatgtggctgctctcagatctttccaagcaagccagtcatttgaagaggttttcttttcatgctggagggcaggctaagatcaatgagtggaagagagaaaggctgttttagctcaagttaaaggaacaccttctagccatcaaagccgcccaacagaggcaagggccaccacacatgagagagcgctctgtccttaaagggaattctctgttgagtgggaggtgaacaccctggttcttccaactcaggaattctcgtggctgggctgggtcagtgatggctttgtctctttatgtctaaagtgccctatggctgctgaaggttacctaaccattctttaaaaggagaatgaccctccatgggaatggccagcctgccaactgtgcaattgaagaagacccgatggatcaaccccatgtctcccttggggagaaagtgcataaaccaggggtctctttttttttttcaacaaaccattgagctgttcttggagttcatctctggagaggttatacattattagaagtttgattattattatagtttgatcaatttatttgtcttagagatccaatttttactaattccctagttttttatttcagcatctgaatgtctttctccctagcacagtgcatacaatcagggccttgggtatttccagtgataactttccttggagaggatctaagaaaagcccagatttcggtagccatctccctccaaatatgtctctttctgctttcttagtgcccattatttccccctctcctttcttctgtcactgccatctccttcttggtcttcccattgttctttaactggccgtaatgtggaattgatatttacattttgatacggtttttttcttggcctgtgtacgggattgcctcatttcctgctctgaattttaaaattagatattaaagctgtcatatggtttcctcacaaaagtcaacaaagtccaaacaaaaatagtttgccgttttactttcatccattgaaaaaggaaattgtgcctcttgcagcctaggcaaaggacatttagtactatcgattctttccaccctcacgatgacttgc

SEQ ID NO: 363 - DNA, 154 bp, homo sapiens
gagagtgatgggagtgagaacctgtgaatctagatgtctgttcagtggttccaagggtgaagtctgctgtattgagcctctgcattctaagcctgagttgaaagatcatcccatcacacagttttcattattggccatggcatccttgacaaaa

SEQ ID NO: 364 - DNA, 91 bp, homo sapiens
agagtggatcatctcctgaaacaagattgtcttacagaagcgttggctcttgcgtggtctttccatgaaggaaaagcaaaagcagtagtgg

SEQ ID NO: 365 - DNA, 129 bp, homo sapiens
gagccaaggcaaagagggaccactgtgctcatggacccatcgctgccttccaaggaccatttcccagagctactcaacttttaagcccctgccatggttgctcctggaaggagaaccagccaccctgag

SEQ ID NO: 366 - DNA, 80 bp, homo sapiens
aggccagcactgtgacacatggtcttatgtgctgataccggcagcccagcctgggcagttcactgtggaccacggtgtgg

SEQ ID NO: 367 - DNA, 115 bp, homo sapiens
gggctcctcatgatttgaagtgtgtaactaacaatttgcaagtgtggaactgttcttggaaagcaccctctggaacaggccgtggtactgattatgaagtttgcattgaaaacag

SEQ ID NO: 368 - DNA, 85 bp, homo sapiens
atttacatgatcggagtaagtctaggagcccacatatctgggtttgttggagagatgtacgatggatggctggggagaattacag

SEQ ID NO: 369 - DNA, 168 bp, homo sapiens
gatttcagtattttaaatgtgaccaccagaggtctgtatacctgtacctgtcttccctgagagagagctgcaccatcactgcgtatccctgtgactcctaccaggattataggaatggcaagtgtgtcagctgcggcacgtcacaaaaagagtcctgtccccttctgg

SEQ ID NO: 370 - DNA, 96 bp, homo sapiens
gctattatgctgataattggaaagaccatctaagggggaaagatcctccaatgacgaaggcattctttgacacagctgaggagagcccattctgca

SEQ ID NO: 371 - DNA, 1074 bp, homo sapiens
gcctcagctgtgtcggtatgatcttgtcctgatggaaaacgttgaaacagtcttccaacctattctttgcccagagttgcagttgtaactgttgccaggacacatggccataaataatagaaagaaagctacaaccacaggctgtttgaaagcttcacctcacctttctgcaaggcagaaaaagtatgaaaaaaaccaaggcttttttcagtagcgtcctatggatgtcacattgtacatcaaacaaccttgtgattataaaacgatcctgggaaggagcccctaactagggcaagtcagaaatagccaggctcgcagcagcgcagcgctgtgtctgctgtgtcctggggcctcccttgttccgacctgtcaattctgctgcctgtcacgcgggtggttctgcccatcgcggctgcgggtcaagcatcttcaagggaaggacggactggaggcctcaccgtggactcaactctgcattctccgtgccacattcctccagttcccacacgtagaagggaacgaaactgacgtctacctcatggggctgctgtgtgggtttgggaggcaaaaatctatgaagggttttttgaaatcccataggtgccacatctatgagatgtttgataaatgtgaatatgcttttacatttgggcttatctaatttgcaataagagagcctctctctatcaacaccagcttctctctcgggctgtttgctcagggaaggcaagaaagccacgtgctggccctctgccttctctaaagtgctgttggagcatggaggagctggaggagatggggatggactgacagctaagagggcggctgctgggactagatagtggatgaagaaagaaggacgaggaagccgtggggcagcctcttcacatggggacaggggatggagcatgaggcaagggaaggaaaagcagagcttatttttcacctaaggtggagaaggatcactttacaggcaacgctcattttaagcaacccttaagaaatgtttatgtttctttattaccaatgtaatctatgattattgaaggaaatttagaaaatgcgtagatacaa

SEQ ID NO: 372 - DNA, 109 bp, homo sapiens
gcagaaggagcttctcttgatgacatttacatgatcggagtaagtctaggagcccacatatctgggtttgttggagagatgtacgatggatggctggggagaattacag

SEQ ID NO: 373 - DNA, 122 bp, homo sapiens
gtgagtggtagccgccgctgaggccgagcctgcactggggccacccagccaggcctgggggcagcctctccccagcctccccgtgccaaaaatcttttcattaaagaatgttttggaacttt

SEQ ID NO: 374 - DNA, 117 bp, homo sapiens
gaatttttaccattcgcctgcccaaagaaacccctggccagcattttgaggggctgaacatgttaactgctcttctggcaccaagaaaatccaggacagcaaaaccacttgtggaag

SEQ ID NO: 375 - DNA, 123 bp, homo sapiens
gaatttttaccattcgcctgcccaaagaaacccctggccagcattttgaggggctgaacatgttaactgctcttctggcaccaagaaaatccaggacagcaaaaccacttgtggaagaaatag

SEQ ID NO: 376 - DNA, 166 bp, homo sapiens
gaactatggaggccgcgcccgggacccccccgccgccgccatcagagtcgccgccgccgccatcgccgccgccgccatcaacgccttcgcctcctccgtgttcccccgacgcccgcccggccaccccgcacctcctccaccaccgcctcccgctccctgacgacag

SEQ ID NO: 377 - DNA, 459 bp, homo sapiens
acaagtgcccttgaattccatctgtgacatgcgcgtggctcaggactccaaccttacctccacacacctggagaacaacctcattgaccggcgccgcatcccgcccactgccttctcctgcacccgagcctatcacagcgtggtcctccagccccagcggcggggggaggagggctcctagtcctgcctgccgccttcctccagcacacactctgcttctcgagggggtggaggggcaggctgaggtgtgcttggcctttgcaccactgggagaaaggtctagaagaccccactgcttccagtgcacccacttcccattgactggtgaggccgcttcacttagcaaatggcccaggggcgaaaggtggcactggtttgtacttgaacataataaatgcagcttgggcaggctcaccagccagcctccctccctccaggagctacgctgcatgtcctggc

SEQ ID NO: 378 - DNA, 144 bp, homo sapiens
gttcagtgcctgggctgctgtttttggggttacccgcctcttcatcctcacccttgccgtgctggccattggctttggactggctcgcatggaaaaccaggcatttgatcccgagaaagggaacttcaacactttgttttgcag

SEQ ID NO: 379 - DNA, 258 bp, homo sapiens
tcgaggacaggaaggaaaaacctgcagagaagtcagctgtatccacttctgtacctacaaagccaacagaaaatatctcaaaggcctcttcatgtaccccagtgcctgtgaccatgacagcaactccacctcttccaaagcctgtgaatacttctctttccccttccccagcattggctttgccaaacctggctaatgtggatctggcaaagatcagttccatccttagcagtttaacatcagtcatgaaaaatactg

SEQ ID NO: 380 - DNA, 201 bp, homo sapiens
gggtcagtcctgcatcaagaccttctccaggaacgcccaccagccccagcaacctcaccagtggcctgaaaacacctgcacctgccacgacaacatctcacaaccctctggcaaatatcctctccaaggtggagatcaccccagagagcattctgtctgcactttccaaaacccagacacagtcagcccctgcactgcaag

SEQ ID NO: 381 - DNA, 1035 bp, homo sapiens
ccaaaagcttcaactattctcctaactcatcaacttctgaagtctcttcaacttcagccagcaaggcctcaattgggcaaagcccagggctcccaagcactacttttaaactaccttccaactctttggggtttacagctacccacaatactagccctgctgccccacctactgaagttaccatctgccaatcttcagaggtctccaagccaaagctggagtcagagtccacctccccaagcctggaaatgaagattcacaacttcttaaaaggtaatcctggtttcagtggcttaaacttaaacatcccaatcctgagcagtttggggtccagcgccccatcagagagccatccctcagacttccagcgtggccctactagcacctcaatcgacaacattgatggaacccctgtacgggatgaacggagtgggacacccacccaggatgagatgatggacaagcccacatccagcagtgtagatactatgtccctgctttctaagatcattagccctggttcctcaacacccagcagtacaagatcaccaccccctgggagagatgaaagctacccccgagagctctccaattctgtatctacatatcgaccctttggtctgggcagtgaatctccctataagcagccttctgatggaatggagagaccatcttccctgatggactcttcacaggaaaagttctacccagatacttctttccaagaagatgaggattaccgagattttgagtattcagggcctccaccctctgccatgatgaacctagagaagaaaccagccaaatctatcctgaaatcaagcaagctgtctgataccaccgagtaccagccaattctgtccagttatagccacagagcccaagaatttggggtaaagtctgccttccctccatctgtaagggccctcctggactctagtgagaactgtgaccgtctctcatcttcccctgggctatttggtgccttcagcgtaagagggaatgaacctgggtctgaccggtcaccatcaccga

SEQ ID NO: 382 - DNA, 140 bp, homo sapiens
gtaagaatgattcatttttcacccctgactccaaccacaatagcttgtctcaatctaccactgggcatctcagtttgccacagaagcagtacccagactctcctcacccagtcccacatcgttcccttttctctccgcag

SEQ ID NO: 383 - DNA, 1428 bp, homo sapiens
aacacccttgccgctcccacgggtcacccacccacgtcaggcgtggagaaagtcctggcctccaccatttccaccacgtcgacgattgaatttaagaatatgcttaaaaacgcctcacgtaagccctcagatgataagcattttggccaggctcccagcaagggcactccaagtgatggtgtcagtctctcaaacctcacccaacccagcttgaccgccactgatcagcagcaacaagaagagcactaccgcatagaaacccgcgtctcctcctcctgcttagacttgcctgatagcacagaagaaaagggggcccctatagaaaccttgggttatcacagtgcatccaataggaggatgtcaggggagccgatccagaccgtagagtccatccgagttcctgggaagggaaatagaggacatgggcgtgaggcttcaagggtgggttggtttgatctgagcacatcaggtagctcttttgacaatggcccttcaagtgcctctgagttggcatcccttgggggtgggggcagcggaggcctcactggctttaaaacagcaccatacaaggaacgggcacctcaatttcaggagagtgtcggcagctttcgttccaacagtttcaactcaacatttgagcatcatcttcccccatcccccttggaacatgggacacccttccagagagagccagtggggccatcatctgccccacctgtccctcctaaggatcatggtggtatcttctctcgagatgcacccactcatctaccctctgtggatctttcgaaccccttcacaaaggaggcagccctggcccatgctgccccaccccctcctcctggagagcacagtggaattcctttccctaccccacctcctcctccccctcctggggaacatagcagcagtggtgggagtggtgtccccttttctactccaccccctcctccaccccctgttgaccactctggagttgtacccttcccagccccaccactggcagagcacggagtggcaggggctgtggcagtatttcccaaggaccatagttccctccttcaagggaccctggctgagcattttggggtactcccaggacccagggaccacgggggccccacccaacgggacctcaacggccctggccttagccgtgtacgagagagcctcaccctaccctcccattctctggaacacctgggcccaccccatggaggaggaggtgggggaggcagcaacagcagcagtggcccccccttgggtccctcacacagagacaccatcagccggagtggtataatcttacggagtccccggccagactttcggcctagggaaccttttctcagcagagacccatttcacagtttaaagagacccaggccaccttttgctaggggccctccgttctttgcaccaaaacgc

SEQ ID NO: 384 - DNA, 98 bp, homo sapiens
atttcttcctgaagtccaggctccaagaacaagatgtctggagaagatccacttctttctatacccacatgtgcaacccctgggtctccctgttgggg

SEQ ID NO: 385 - DNA, 163 bp, homo sapiens
attggaactagatggaaagatgttgtctccaaatgcattcgatgccactttcagccactacttttgttttatgcaaacccagatggcacagcagtttctactgaggatgcactcaggcaggtcatcagctggtcacattacaaatctgttgcagaaaatatgg

SEQ ID NO: 386 - DNA, 70 bp, homo sapiens
gtcacgctgctccagttccacccaaacccagggaaggagacccagaaaaaacaaagatgccccaaagaag

SEQ ID NO: 387 - DNA, 144 bp, homo sapiens
gtaatgtgtgaagtgctgttttcagctacagcaaatgtgcatgaaaatttaacatcttccatccaaagcagattaaaggctcagggtcacagccacatgaatgcccaggctctgctggaagaaggtgatgcagggagctcggag

SEQ ID NO: 388 - DNA, 202 bp, homo sapiens
aattcatctgagaaatctgatatgcttggaaataccaattcttcagttttgtattttatactgggtctacactttgaagaggataaaaaggcgttagaaagcatccttccaacagcacccagcgctggcttaaagagacagtttccggatgacgtggaggatgcacctgacctgaacgcccctggaaaaattcccaagaaag

SEQ ID NO: 389 - DNA, 94 bp, homo sapiens
atctctcacttcaaaggagcccaaactctgagacagaagaaagtcaggggctctcgcttgatgtaactgactttgagtgtgccctctgcatgag

SEQ ID NO: 390 - DNA, 115 bp, homo sapiens
attgctctttgaacctgtcactacgccctgtggacacacattttgcctaaaatgccttgagcgctgccttgaccacgccccacactgtcctttgtgcaaagacaaactttcggaa

SEQ ID NO: 391 - DNA, 163 bp, homo sapiens
gctttcagagtatggatgcatgctggagattaaggacgtgagaacgtttcctgatggaagttctgttgtagacgcgattggcatcagtcggttccgagtgctaagccaccgccacagagatggctataacacagcggacattgaatatcttgaagatgaaaag

SEQ ID NO: 392 - DNA, 150 bp, homo sapiens
gtggagggtccagagtatgaagaacttgccgctctccacgattctgtgcatcaacagtctgtttcctggttcgcgtctctccaggatcgcatgaaagaacaaattttaagtcattttggggtaatgccagacagagaacctgagcctcag

SEQ ID NO: 393 - DNA, 10940 bp, homo sapiens
cgtccattcattgtgcttcatcaagtgcttgtggatgaggttccaaaatgggacgcttgccaaacattgagtcctcctcaaaaatgacaattctgtgtctggtgggatctgaccttgtgtgaggttagcctgaagtctgaatggagcccatagttggaaaacaacctaagaaaatctcttagaagcaggtgcttggggaatgcagttcactgacagcacaggaccctgcagatggtttacatgtggtttgggtttcacgagaaagaaggattcacttcccagtcagcatctggctctgccagatggtaaaggcgtgctttagtgtgtagacaatatgggggaaccacgtttttatctggaagtggatttcttagaacacaggctaaccaaaactacgcttaggctttgcgtgttgctgtgaagttgtctgtgaaatcgaataatcacaccattgttcagtgcaggagcccaaactagtccttacccaagaagtagtagcctctggatagaactgtgtttaatgtcctgttgtagtcccaggtgttgtaaattgcatgttgtaatcaaacgaatgtcaaaacataagaaagtataccttggatatagaaaaacctgagaacagtatcattcacttgaggatatatatatatatatttacacacaataaagtgagttaaaattgtatatgcattgggatgtcaaacataaaaccaccaagtgcaaagaatgctttgaaagtagaaccttgtctcattgatcagtggttactaagcatttaggaaacagtcatctttttctattgggatttgcattagaaattagccccatcagtaaatttcagtttactatccatttattaatatacaaaaacattaaaaaatctgccatgccatggtaatctatctattccaggaaaatagatcagaatatatgaagggaggatggccggtatgggatgtaaatattcagtaagtttttctccaacaccaacctaaaattatctttatttttttgaaagaaagttttacggtgatcagaccaggtgttatctgttaaaggacagtagtatgacttccatcctgtaatgcacgttggtgatattgattggaataactgaatctgagatactcatatgttctctctggcccaaccagctggtgcatttggttggcttcctcgtctgtaaggtggacattcttaaccagagggtcacgaccacatcacagctgcaagttttgctttgttcattgaaggtgattttttagttttaaaatatatatatctcataatatacaatgttgatggtatatcaatccatcaagcattttattagaaagactttataatattattaatttttctttagttttcagtagataaagtagtttcagtagataaagactttataatattattaatttttcttttagttttcagtctcatcatgggagttagaaaataccagcatcacttaattgtaaagagaaaatgtgaaagtattttccaatccaatgtttcaagtgcctcacttattgtgaatctcaaatatcatcttgagatggtttaatggcagtttggccaagtgatttctaagagattttattaatttatgcattaacaatagtattcaactttcttaggcattataacctaacccgatcatttgattggaataactgaatcattcattgattggaataaaataaagaaaacaaaatgcatctcacatgccagaagacctttaccagaagtggttcatgaaaatcagtatgcattgaccattctctgcctttcccttttggaaaatttttttgtaccttcatatcacagctgaatagttatcaatgccatttccatttggaagtatctaaataaccacaatgtgatcacagcttctccacgagccttcctgaagtaaagctggtatgcttgctataatacttggaacctgttgctttctcttcctcactctttaaatgtcataaggtcacacagttcttggaatcagaactattcagttatttcttcatgattctgcctggctttctgagtcttctccttcggcatgttatgtaaaagtctgaacaacaatgatgacgcagacgagagcaacatacgtttttattttttgataggtttttatttttttgataccagagggaattaactatgatcacatcgtgctctctctgttcagcatcacacgcccacatactgcagtgtgggagtagattggtacttactgagcacttagaaacatcacgcatcaggaaccgggtcagggtgacagcatcctacggtcatatcatgaaggaagcctcccttacaaacctaaaaaaaaaaaaccccagcttcagtttgctaccaattaagctaaagcagaacggtctgcttcctcctccgcctgcccccttctgcctcttttctgttggggagatgccaattctaaggatttctcagaaaataatgtaatctctcattcaatgtcaggacattccagcttaattgtactgaaagtgctccaataggcatgctctcgttacattggaatcaaagcagaaaagagtaatagcagcgtgggggagtcatttttcttcttgaggaacaatacatgctgttctgtatttttccattcatcgcccaaacctgcctgtgtctttgaaatgcaagtaagatcccaaaggcagctttctgaaagcccgtggcatttttagcttatcccatcccccgtgtgtgtcctcatcttcagaattgaaagttaggatactttttaaagtttcaaaagaaaataatttaaaatgttaaggtttatccatagaaacacaataattcttaaccaattcatgtaccaaattcaacagtattatctcatttttagggtcacaaatgtgtcctttttgcacttaaacctcctccctctctcaaaaacaaaaacaaacaaacaaacaaaaaacacaccaagactgtttaacaaattgtatctcaactcttaatatatgttaagtcttaccacaggacatttgaatcatcaaggaacagttgaagtttatggatggtagaataacaatgaactatgatattatcactttattataaactttttggaaaattggcagttgctaccatcgaaatactccattgcctgtgttacatagaatttgttataatttttaagggctttaaaaaaatacccatctgtttcttctccttcttgttttcttttgtgccccaccacttaaattacttgggtaaataccactcttcaaaatttgaatactgtctatcaaataagaagaagtgtgaaagatatgaagaagaaaggtgatagcaaattagaagaaaataaatgtgggtgatttcttttagttgaaagcacagagttttatttttccccagtataactattgagtagggtagggaggtccctgtatccccatttttattttttttgagatggggtctcactctgtcacccaggctggagtgcaatggcgcaatctcgtctcaccacaacctccgcctcctgggttcaagtgattctcttgccttggccccctgagtagctgggattacaggcacgcgccaccacacccagctaatttttgtattttttttttttagtagagatggggtttcaccatgttggtcaggctggtctcgaactcctgaccccatgatctgcccgcctcggcctcccacagtgctgggattacaggcgtgagccaccgtgcccggcccatctccccatgttttagcatagacagcagcggcattcagtggaaggagttcttccccttcaacatcgtccttcccaaaggccacattctgattctattgttcaccaaaaactaattcccagggaagcccgctctggatgggaaggatgagctgcgtgagccatcgcccacacagggagataaccgaacccctttgccagcatcaccccctctctactggggtgagacacttcattcagagcactggctctgactcccaggggaaagtctttttccctttacaactcctcacctcagcctcaccttgactttcacttctcaactgctgaattctataaaatagccttttagcagtgactcagcttttaggtgagcacagataatgaagaaattgaagtgccaccattgcactcccccaagttggggacataaactcgaatttgcacccaggtctgataaaagtaactgattgaggacgcagaaaggtggatgccattgacagcgttgactgttctcacaattcctacagaaatgaaagaaggagaggcccagcagaccacaccgggccatgaggggaagcatgggagacaggcagaagcaggagtgaggaggaacgtggctcagaccctctactgtatgttctgcaggaaagggcagggaaatagctaagactggctggtctgaataacagctcagactctaggctatacaggtggtctctagccatctggcacctagccccgggtcgttagggtgggggatattgcttcctaggttgtaagagccagatagaggaggatgggctctggattggtcactttgctcccagggtagccctttgctgtatctaagaattggctcaccctgggatgggcagtttctcctgggtcagcgaggcccctgagatgtcaaaatatcataaaatatagaaatcatgattaatacaattggtcctctgctgttttgacatactaaggtcacttttgatagcagaaacttttaaagagtagggcatacgttgccctgaagagcatagaaaacattttattctgagagtagaaaggaggaagacacgtggagatacaggagtcccttaggctggcccatagctgagtctctcatgtgtgtggcattagtcaagaatgtagttctgcagtttgcggggggggggtctctaacatttcttggtgcagttgttaacatgtgttaaacatatgagtctgatgatgatatcgcggtgtttctcggctcggggtggcaggtctggcggttgtgtggctgggggtgtgcactgatagttcagtaaaggataagtagagactcgtggtgttcatgtgtcaccacagggagggagtggctgtggagacccaggacaggaagtcgcggtggaaagcaggctggggtggtaggagaaggcaccgggggtgggtgctcctcccccaggggttgaggagttgactgatagggggcagttacctcctgggaaagcaagacgctcccttaagttttagaatggtacagagagtagtcccttctaccagagtttccatttagacatgggagtgtagggggccatgtttcggcagaagcagggcatgtatcagcattagtggactcctcagtattaaaaggaagaacagcaaacatgtcatgtgcccactttgtgcctcaggagaggtagcccgtggacatcgctagggcagaggttttaatatctgggacaaagttatctcccacggcgaggtcgtggctgggaccatgtcatcttcagccgtgagctgctgggctgggcaatgtcttctgggatgtgagacgtggggtctcctttctaagcggacccccactcgtgctgatgtgggtcccagcatttgccgcgtttggttgaaacagtggcttacctggagggtctgggtctctggatcataaaccagcgaaagcccccacatcagtggtgacaagatcaatagaccccagaatgtgtgcagctgtgcttgatagggttttggttcttgctttttggtggaagtttaggctcaagtgggtcccgtatcggatgagcagctgcacggcctgtgggagccctggggttagtgggtccaggagagaatgtgttctctctctgggctccagccctgatggggtggggataagatgcaagatttataggaatagggggaaacattatggaaggccccaatatgtaatgagcttttatatacaagaatgcaagacatagccctctttttatgtttaaaggggactatggtaatgttttttaaagtttgtgtgaggtgtgagtcaaccgaaaggccctaaattaagagagattcaggtttgtggtgcaagatagaggcccatttcggggtctgtttgcagaccagaatttgaaatggagttgtttgaggagaccattgtgtgtcttcgtgaaaccgattgtgtggaacctatcagggctgtggaagttgtagcgaatgccttttcccagagcccagcattgtggattccgagaagtggcatgtgtgtctcagtgacttccagtgatgcctgccactctgaagagatgaaggagtgtcctggcaagacctggaatcccagctgtagcacctgtggagagatgtgatttagatttggatttggggtatctgtgaggaagagtcccagagtttttcatcctgaagggggcaactcttgggcagtggtccaacagttggccataaatgtgaagctggggcgctgtgctgaccagggcatccagcgttaagacgactgcctaactttggccacatgcgttgttttttgggttccgtttcgtatcagggcatgctggctaaaagatgggaagcagcaacattctcttgggctccatcatttatatgagggttggcaatgccatctgtaccctctgtgaactcccactgctgttcactcagttaatctaagggacaccaaggggtctgcgggaggatgacctcacaagggactgaggacatgggagaccaccctcctgtgggtagaatacaccagagggcccaggcttgactctgtcctgtgttaaggaggcatcggtagttttgctgagcttgtcaggtgtttataatggacagttggagattggggtcctaggcttaggtctttgagagcctctgtgtccaggagagcccagtaggcatccagccatttactgctttaatagggtagcatgaggctgagagggatagtttcttgcatccaaagcccttaggcaccttatgtccatcctaagtgggccagagactccagggctgcatagagcttgccagagcttctataatgaaggggtctctgagggcaccaacaggagtgcccatggattttaaggtttgattatagaggttgtttatggagggtgccccatttaaagcaagctgatttgtaagcagcagcaagaagattaagtaaaatttgtaaatgaggactacattgccatcagaacctcgaaagtatctaaagatgttggacttgtatgtccttaatgagtgtgacaatgagtttcctcatttgtgctcctggagaaggcggatgtggtgaagaccctgtctgcagacattgtgtgccatggcaaagccgtggagctccctgtgggtggcctgcaaaggtgtatgtgcccctcagggcagaaggcaaacggcagccaagaagctgtgcaagtagacacttaatgggacatgttagccaaatctgtaagagcaaaatattggccagttatttattgtgtagaattaataattttaataataatggaaattgggtaatggatgggactgcagcaataaggttgtagtaatccaccatgaggcacacttgtttttttccaggtttaaggataggaaagattgggctgttcaatggagaaacaagggtataatcacccctttattaattagtaagttttaatccttgaatacctcatattaactgttttaactggaggtccatggggcatcattttatcaagctagtttataactgccaaagactgactttaattttaatttattatttgttttattagagtgtctgtgttcaatatgggatattagggcgttgggtactatgaccacaggaaatttagacaggctacagttaaagtgaagcataccttacccatccaccccccattttatatttagttgcctttttaaaaagattataggggtacaatgtttagatttagtgggatctccaggtataactgtaatttgagccccagtgttaagactatgaagctttgtcaatgggtacattttagcaaatgttacaattaatttagaacctaagttatggagacacaaaagccaataggcacccttttatgttttggttaaatgtttcagtatatacatcttatttatttgtaatattagtatataatttgttgtatacatttttagtgtataagcgttggatttctaattggatcagattagggaccttccgtttagctgcatatgtacatatacatgtacaatttattatatatttgcgttaaaatagcctatctgcatgtgtatatatgtgtgtatgtgtatgtatatgcactcacacgcataaatacacagtctatttagttacctttaatgttttttcccttgtacctaggctttttctcgctttttcctttttttctgattttgtggcaatttagttggaaggaggcggtcccagcatgttgacaggcagggtgttcagagtgcccaggcacactggtggggggtggttacaggctcacgtagctcaggggcttctgcaggtctcaggggagtgggaacaaagtgtcccaccccttccccttttcctcaaacctcaagccactggtctctatggatagatcctttgcatcccaccggattgaggaatgagtcacaacagctgcaaggctcttaaagcaacatttaaaccttttggcggctgtcatttctgtgaggagggtgcctctcaccggccgcatggccggaggatccctgcagcgctttggagaccaacacccagatcctttgcccaggagtgcgattaattcctcactggatgctgggggagggcccctcaggtgagcagcccaccactgacttcagcgttgctggctcggttatcagactctcatccaacacaagctcacagggaaagccgttccttgctccttgtggagggagctaccgtcattgccctgagaccaccagccaagaaagtaggtatgtccaggtagggaattcagagggacccagtgcatccaattatacaattatacccagaaggtcctgtgtaggggactgcgattgacatcaccctagtctgcagcaccaaggactgaatgagctcagtcctcttataatttaggctggactgtcacagacactggcagacacagcatacgtggtgcagccaaagtgcaaacatgccagcagcggccatgctccccagggtgggggtccagttagtaagccacgcgcagccaagaggcgaggcatgccctgtgccacacacggactcaccctgctcactgtgcccgtggtatcgaaatgtacccacgtttaattcataaaggagaggctgctgtcattgaaagaaaagtttgttacttgcatttctggagaaaaggagcgcaccaggccacgcagggccacaggaggaggacgcaccagagtggtcaggaggcagaactaggcgagcagctttccactgtgtctccatggcaaaggcgaagatgggcgggggcagagtgtaggattggcaggtttgaatgtcttgggcagtagctacaggggtggtctccagctgcctggtgcctggccctgggtgatcagggtgaggggatactgccttctgcagtggaagagtcaaatcgaggagatggactctgagttggttagtgtgcaaaggtgcactcccaagggacccctttgctatctctaagaattggcctgccctgggaagggcagtctctccccagtcagtgaggtccccaagatgtgaaaacattatacattataaaaaagcatgattaatataagctcattctagcatttcaggttacagcttctagaagaggtttgtagtctcaaatgagtaggtttttcctctagagaggggcgggcctggaccttcaagcaccccttggtgtgtttaggagctcaggagcagaagcacctgcctgcagccctgcagctaaggaagttctctcagtcactcagagcagggaggggctgagagagtcatgtgaggctcccggggtactacgacagccctcgaggtgaaggattggccctgatcataatagagaaccctgaggaagtttactgtcatgagtctcggctggttggcgcatgtgacctttgaaggatgaagatggagtttgcaacatgagtatctctaaccttttgcttttcagggatcattttcaaaaattgcattggggccttcgttatttaccatagtattttcactttcatagttttgtcacctttttgtactgtgaacagttcaaccagtgaccgacttctctctcatgctgtttaccccacacacaatttcccactcaattctgaaaataagaacctgttaataggttggaaagctgtgtactctattcatatattgttctttcatgctagtggagagtggtgtcattagcatcttaattttagagttgtgaaatgattttaccaattaggaattgaatgtgtattttttttctgtttaataagaagagcaaatttgaataaataagctggtgtagataaacttaataatcatgctttttcttgtttggagataggtgatgtgttgtcatatcctgtgatacaggtcactcatctggccttctgtttctgaagtttaagtctggtttgaatatgtaataatactactcagcatttcttgttgcctaagtgagacgaaacttaaatgttatgatatttacttcatgtattcttgtactgttcatttcaattaattggtattgtatatctaatatgtgatatttgaactgaataaaacttacagtgttgtaaatgttctttaataaataatcacacctaagtaataggctagactgatgagaaattagatcaaatcagtgcctaggcctttgtgtttgtgtcgtagggatgtactaacacatcaccacagactgacggcttttcacaataaatcagttttcttcgctca

SEQ ID NO: 394 - DNA, 34 bp, homo sapiens
agcaacacggagcacaggtctctgctgctgatga

SEQ ID NO: 395 - DNA, 231 bp, homo sapiens
ggccagagcctgtgctgctgttcgccaatcgcatcgacatccggcaggtgctgccacaccgctctgagtacacactgctgcttaacaacctggagaatgccattgcccttgatttccaccaccgccgcgagcttgtcttctggtcagatgtcaccctggaccggatcctccgtgccaacctcaacggcagcaacgtggaggaggttgtgtctactgggctggagagcccag

SEQ ID NO: 396 - DNA, 218 bp, homo sapiens
taccatttactggacagactggggcaacaccccccgtattgaggcctccagcatggatggctctggacgccgcatcattgccgatacccatctcttctggcccaatggcctcaccatcgactatgccgggcgccgtatgtactgggtggatgctaagcaccatgtcatcgagagggccaatctggatgggagtcaccgtaaggctgtcattagccagg

SEQ ID NO: 397 - DNA, 132 bp, homo sapiens
tgtctacaccatgtgctatggagaatggcggctgtagccacctgtgtcttaggtccccaaatccaagcggattcagctgtacctgccccacaggcatcaacctgctgtctgatggcaagacctgctcaccag

SEQ ID NO: 398 - DNA, 304 bp, homo sapiens
gttttaacaagtgcggctcgagaaatggcggctgctcccacctctgcttgcctcggccttctggcttctcctgtgcctgccccactggcatccagctgaagggagatgggaagacctgtgatccctctcctgagacctacctgctcttctccagccgtggctccatccggcgtatctcactggacaccagtgaccacaccgatgtgcatgtccctgttcctgagctcaacaatgtcatctccctggactatgacagcgtggatggaaaggtctattacacagatgtgttcctggatgttatcag

SEQ ID NO: 399 - DNA, 135 bp, homo sapiens
gtacctcttctggacagactggggccacattgccaagatcgaacgggcaaacttggatggttctgagcggaaggtcctcatcaacacagacctgggttggcccaatggccttaccctggactatgatacccgcag

SEQ ID NO: 400 - DNA, 109 bp, homo sapiens
gatctactgggtggatgcgcatctggaccggatcgagagtgctgacctcaatgggaaactgcggcaggtcttggtcagccatgtgtcccacccctttgccctcacacag

SEQ ID NO: 401 - DNA, 145 bp, homo sapiens
caagacaggtggatctactggacagactggcagaccaagtcaatccagcgtgttgacaaatactcaggccggaacaaggagacagtgctggcaaatgtggaaggactcatggatatcatcgtggtttcccctcagcggcagacag

SEQ ID NO: 402 - DNA, 136 bp, homo sapiens
tgcctggcctggtaccaccagctcctagggctactggcatgagtgaaaagagcccagtgctacccaacacaccacctaccaccttgtattcttcaaccacccggacccgcacgtctctggaggaggtggaaggaag

SEQ ID NO: 403 - DNA, 68 bp, homo sapiens
atgctctgaaagggatgccaggctgggcctctgtgcacgttccaatgacgctgttcctgctgctccag

SEQ ID NO: 404 - DNA, 88 bp, homo sapiens
gggaaggacttcatatcagctacgccattggtggactcctcagtattctgctgattttggtggtgattgcagctttgatgctgtacag

SEQ ID NO: 405 - DNA, 2331 bp, homo sapiens
ggagggcctgaccataactacaccaaggagaagatcaagatcgtagagggaatctgcctcctgtctggggatgatgctgagtgggatgacctcaagcaactgcgaagctcacgggggggcctcctccgggatcatgtatgcatgaagacagacacggtgtccatccaggccagctctggctccctggatgacacagagacggagcagctgttacaggaagagcagtctgagtgtagcagcgtccatactgcagccactccagaaagacgaggctctctgccagacacgggctggaaacatgaacgcaagctctcctcagagagccaggtctaaatgcccacattctcttccctgcctgcctgttccttctcctttatggacgtctagtccttgtgctcgcttacaccgcaggccccgcttctgtgtgcttgtcctcctcctcctcccaccccataactgttcctaagccttcaccggagctgtttaccacgtgagtccataactacctgtgcacaagaaatgatggcacatcacgagaatttagacctggattttaccatgaacctcacatcttgtactccatcctgggccccctgaaactgcttattcgtgattcctcaccagcgtagagctccacctcccctttccccagtaccctcagtgcctgcttctcagtgctgatgcagctgatgacccaggactgcgctctgccccatcacagccagcatgactgcttctctgagagaacttgcccatcaggggctgggacatgggggtgtgggtaaagacagggatgaaggatagaggctgagagaagaaggaagaatcagcccagcaggtatgggcatctgggaaacctccagcctcaagtgtgttagtaacatgaaaaagctttggggggtagttggatctgggtgtctggtccattgctggcagtggacattattcttgccctaagagacactgccttttcagcagcagatactggtgagatgggggtggctcaggctgttcttcctcctcctagaatgtctggagctgtttctacattcagataactgggtcccctatcacaaggctactggctaataggaattccctcctggtgccaccactggccagtacctttcctaagtctttgctcaaattaaccaggttgtgagccagtggcttgagtgaatgttaggccttgggggctgagtctctgaaaagtctaagaagctctgcctagaccaaatatggtatacctcctgacccctctctccctcatgtcctgggattctggggaagagacctagaaacaagctttcaaagaaaaaccagaagttgtcataaatggtcagaaagaacgatcaggttggagacttgggaaacccagggcctaaagagaagtatccatgagggtcaaacttcctgttgaacttcctatgttctttctcaagtgctcagggatctaagttagtggacagcaagcctgtggctacggggtggtgatgttcctcttccagctgtcccctcagctaaggggcttagtttccatgtgggatgccatcacttggttcatgctcattcacacaaagggcacgtgtctcagcctggtatcagggaaattgagacttatttttgccctaaaacgtctccctagctgttcttcgtggggtttttttgtttgtttttttgcctaatttgctttttctgaccaagccttgtggcaccagcaatctccaaagtcctgtggtgggagggctgaataaataaaaatacaaagaggtgggtaaggagtaggaaggtagagagcaccactgatgaggccctcctagcccatggcagacccagacctcttctcccccaggaattagaagtggcaggagagaacaacaggggctgggaatggaggggagaatttctaggggaagtttcctgagttgaaacttctcctgtggttactggtattgagaaatcagctaccaaagtgaaaaaggacaagatcaattcttttctagtcagttctaagactgctagagagagataccaggcccttagccttgctctcagtagcgtcagccccagttctgagcctccccacattacacttaacaagcagtaaaggagtgagcactttgggtccttagactcacgtctggggaggaagagcaagtagaaaagtggcattttcttgattggaaagggggaaggatcttattgcacttgggctgttcagaatgtagaaaggacatatttgaggaagtatctatttgagcactgatttactctgtaaaaagcaaaatctctctgtcctaaactaatggaagcgattctcccatgctcatg

SEQ ID NO: 406 - DNA, 118 bp, homo sapiens
tgtaatggttttaacgttactcactggagagattggactttctggagttatttaaccactatgttcagtattttaggactttatgataatttaatataaatttagcttttcttaatca

SEQ ID NO: 407 - DNA, 554 bp, homo sapiens
atcttgtctggactaccagactcaagagaccaaatcaagcctttctaagacccttgaacaagtcttgcacgacactattgtcctcccttacttcattcaattcatggaacttcggcgaatggagcatttggtgaaattttggttagaggctgaaagttttcattcaacaacttggtcgcgaataagagcacacagtctaaacacagtgaagcagagctcactggctgagcctgtctctccatctaaaaagcatgaaactacagcgtcttttttaactgattctcttgataagagattggaggattctggctcagcacagttgtttatgactcattcagaaggaattgacctgaataatagaactaacagcactcagaatcacttgctgctttcccaggaatgtgacagtgcccattctctccgtcttgaaatggccagagcaggaactcaccaagtttccatggaaacccaagaatcttcctctacacttacagtagccagtagaaatagtcccgcttctccactaaaagaattgtcaggaaaactaatgaaaa

SEQ ID NO: 408 - DNA, 119 bp, homo sapiens
tgtaactcagctccacctgaaggataattgtttatctgatgctggggtgcggaagatgacagcaccagttcgagtgatgaaaagaggccttgagaatctaacattattagacttatcat

SEQ ID NO: 409 - DNA, 209 bp, homo sapiens
ttccctgagcagcggacgttcatgctgggagggcggcgggttggaagcaggtgccaccatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcgcg

SEQ ID NO: 410 - DNA, 110 bp, homo sapiens
gtgggttggtcacttctgtgcaaattaatagaagtctgtccaggtacaatgcaaagcttaatgggaccccaggatgttggaaatgattgggaagtccttggtgttcacca

SEQ ID NO: 411 - DNA, 89 bp, homo sapiens
attgattcttaaaatgctaacagttcataatgccagtgtaaacttgtcagtgattggactgaagaccttagatctcctcctaacttcag

SEQ ID NO: 412 - DNA, 135 bp, homo sapiens
tctcagaggagcaactgactgaatttgttgagaacaaagattatatgatattgttaagtgcgttaacaaattttaaagatgaagaggaaattgtgcttcatgtgctgcattgtttacattccctagcgattcctt

SEQ ID NO: 413 - DNA, 132 bp, homo sapiens
gcaataatgtggaagtcctcatgagtggcaatgtcaggtgttataatattgtggtggaagctatgaaagcattccctatgagtgaaagaattcaagaagtgagttgctgtttgctccataggcttacattag

SEQ ID NO: 414 - DNA, 120 bp, homo sapiens
gtaattttttcaatatcctggtattaaacgaagtccatgagtttgtggtgaaagctgtgcagcagtacccagagaatgcagcattgcagatctcagcgctcagctgtttggccctcctca

SEQ ID NO: 415 - DNA, 143 bp, homo sapiens
ctgagactattttcttaaatcaagatttagaggaaaagaatgagaatcaagagaatgatgatgagggggaagaagataaattgttttggctggaagcctgttacaaagcattaacgtggcatagaaagaacaagcacgtgcag

SEQ ID NO: 416 - DNA, 80 bp, homo sapiens
gaggccgcatgctgggcactaaataatctccttatgtaccaaaacagtttacatgagaagattggagatgaagatggcca

SEQ ID NO: 417 - DNA, 55 bp, homo sapiens
tttcccagctcatagggaagtgatgctctccatgctgatgcattcttcatcaaag

SEQ ID NO: 418 - DNA, 52 bp, homo sapiens
gaagttttccaggcatctgcgaatgcattgtcaactctcttagaacaaaatg

SEQ ID NO: 419 - DNA, 130 bp, homo sapiens
ttaatttcagaaaaatactgttatcaaaaggaatacacctgaatgttttggagttaatgcagaagcatatacattctcctgaagtggctgaaagtggctgtaaaatgctaaatcatctttttgaaggaag

SEQ ID NO: 420 - DNA, 113 bp, homo sapiens
gcatgccagaagaatccagggaggatacagaatttcatcataagctaaatatggttaaaaaacagtgtttcaagaatgatattcacaaactggtcctagcagctttgaacagg

SEQ ID NO: 421 - DNA, 145 bp, homo sapiens
ttcattggaaatcctgggattcagaaatgtggattaaaagtaatttcttctattgtacattttcctgatgcattagagatgttatccctggaaggtgctatggattcagtgcttcacacactgcagatgtatccagatgaccaag

SEQ ID NO: 422 - DNA, 140 bp, homo sapiens
aaattcagtgtctgggtttaagtcttataggatacttgattacaaagaagaatgtgttcataggaactggacatctgctggcaaaaattctggtttccagcttataccgatttaaggatgttgctgaaatacagactaaa

SEQ ID NO: 423 - DNA, 171 bp, homo sapiens
tttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcag

SEQ ID NO: 424 - DNA, 259 bp, homo sapiens
gtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaa

SEQ ID NO: 425 - DNA, 189 bp, homo sapiens
atatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaag

SEQ ID NO: 426 - DNA, 218 bp, homo sapiens
ggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaa

SEQ ID NO: 427 - DNA, 251 bp, homo sapiens
actctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaagg

SEQ ID NO: 428 - DNA, 149 bp, homo sapiens
aaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttg

SEQ ID NO: 429 - DNA, 94 bp, homo sapiens
ctgctatgcctttcttgcctccttctatgacaatcctaaaattatctcagaacaaattttcctgtattccagaagcaattttaaatcttccaca

SEQ ID NO: 430 - DNA, 128 bp, homo sapiens
gtcgtgaggaattctatagtactcatccccattttatgacgcagcgagcattgtaccttgctgtctatgacctcagcaagggacaggctgaagttgatgccatgaagccttggctcttcaatataaag

SEQ ID NO: 431 - DNA, 219 bp, homo sapiens
gctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaag

SEQ ID NO: 432 - DNA, 202 bp, homo sapiens
atccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcag

SEQ ID NO: 433 - DNA, 89 bp, homo sapiens
gagtccttcttcattttcaagacccagcactgcagttaagtgacttgtactttgtggaacccaagtggctttgtaaaatcatggcacag

SEQ ID NO: 434 - DNA, 155 bp, homo sapiens
tttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagag

SEQ ID NO: 435 - DNA, 147 bp, homo sapiens
aacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaag

SEQ ID NO: 436 - DNA, 192 bp, homo sapiens
gctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaag

SEQ ID NO: 437 - DNA, 147 bp, homo sapiens
gagatctcttagtaaatccagatcaaccaaggctcaccattccaatatctcagattgcccctgacttgattttggctgacctgcctagaaatattatgttgaataatgatgagttggaatttgaacaagctccagagtttctcctag

SEQ ID NO: 438 - DNA, 101 bp, homo sapiens
gtgatggcagttttggatcagtttaccgagcagcctatgaaggagaagaagtggctgtgaagatttttaataaacatacatcactcaggctgttaagacaa

SEQ ID NO: 439 - DNA, 191 bp, homo sapiens
gagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgag

SEQ ID NO: 440 - DNA, 161 bp, homo sapiens
atacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccag

SEQ ID NO: 441 - DNA, 171 bp, homo sapiens
ggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctg

SEQ ID NO: 442 - DNA, 101 bp, homo sapiens
atccagttaaagaatatggttgtgccccatggcctatggttgagaaattaattaaacagtgtttgaaagaaaatcctcaagaaaggcctacttctgcccag

SEQ ID NO: 443 - DNA, 195 bp, homo sapiens
gtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgag

SEQ ID NO: 444 - DNA, 194 bp, homo sapiens
gaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaag

SEQ ID NO: 445 - DNA, 73 bp, homo sapiens
caaacaaaaaaattttcttttggttggaaccgctgatggcaagttagcaatttttgaagataagactgttaag

SEQ ID NO: 446 - DNA, 185 bp, homo sapiens
cttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaact

SEQ ID NO: 447 - DNA, 153 bp, homo sapiens
gttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaag

SEQ ID NO: 448 - DNA, 209 bp, homo sapiens
ggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctag

SEQ ID NO: 449 - DNA, 72 bp, homo sapiens
gaagccttaaaaatgtcatgctggtattgggctacaaccggaaaaatactgaaggtacacaaaagcagaaag

SEQ ID NO: 450 - DNA, 215 bp, homo sapiens
agatacaatcttgcttgaccgtttgggacatcaatcttccacatgaagtgcaaaatttagaaaaacacattgaagtgagaaaagaattagctgaaaaaatgagacgaacatctgttgagtaagagagaaataggaattgtctttggataggaaaattattctctcctcttgtaaatatttattttaaaaatgttcacatggaaagggtactcaca

SEQ ID NO: 451 - DNA, 1426 bp, homo sapiens
ttttttgaaatagctcgtgtgtatgaaggaatgttattatttttaatttaaatatatgtaaaaatacttaccagtaaatgtgtattttaaagaactatttaaaacacaatgttatatttcttataaataccagttactttcgttcattaattaatgaaaataaatctgtgaagtacctaatttaagtactcatactaaaatttataaggccgataattttttgttttcttgtctgtaatggaggtaaactttattttaaattctgtgcttaagacaggactattgcttgtcgatttttctagaaatctgcacggtataatgaaaatattaagacagtttcccatgtaatgtattccttcttagattgcatcgaaatgcactatcatatatgcttgtaaatattcaaatgaatttgcactaataaagtcctttgttggtatgtgaattctctttgttgctgttgcaaacagtgcatcttacacaacttcactcaattcaaaagaaaactccattaaaagtactaatgaaaaaacatgacatactgtcaaagtcctcatatctaggaaagacacagaaactctctttgtcacagaaactctctgtgtctttcctagacataatagagttgtttttcaactctatgtttgaatgtggataccctgaattttgtataattagtgtaaatacagtgttcagtccttcaagtgatatttttatttttttattcataccactagctacttgttttctaatctgcttcattctaatgcttatattcatcttttccctaaatttgtgatgctgcagatcctacatcattcagatagaaacctttttttttttcagaattatagaattccacagctcctaccaagaccatgaggataaatatctaacacttttcagttgctgaaggagaaaggagctttagttatgatggataaaaatatctgccaccctaggcttccaaattatacttaaattgtttacatagcttaccacaataggagtatcagggccaaatacctatgtaataatttgaggtcatttctgctttaggaaaagtactttcggtaaattctttggccctgaccagtattcattatttcagataattccctgtgataggacaactagtacatttaatattctcagaacttatggcattttactatgtgaaaactttaaatttatttatattaagggtaatcaaattcttaaagatgaaagattttctgtattttaaaggaagctatgctttaacttgttatgtaattaacaaaaaaatcatatataatagagctctttgttccagtgttatctctttcattgttactttgtatttgcaattttttttaccaaagacaaattaaaaaaatgaataccatatttaaatggaataataaaggttttttaaaaactttaa

SEQ ID NO: 452 - DNA, 107 bp, homo sapiens
tttcccagctcatagggaagtgatgctctccatgctgatgcattcttcatcaaaggaagttttccaggcatctgcgaatgcattgtcaactctcttagaacaaaatg

SEQ ID NO: 453 - DNA, 168 bp, homo sapiens
gtctcaatgggggcactggggctggagggcaggggtgggaggctccaggggaggggttccctcctgctagctgtggcaggagccacttctctggtgaccttgttgctggcggtgcctatcactgtcctggctgtgctggccttagtgccccaggatcagggaggactg

SEQ ID NO: 454 - DNA, 153 bp, homo sapiens
gatgaaaaggagcagcttatacagtccaagagttccgttgccagtctcgttgggagatcaaaaaccatcgttcagctaaaaccacgcagtccagaccatgtgttaaagaacaccatttctgtcaaggctgtctgtgactacaggcagatcgag

SEQ ID NO: 455 - DNA, 155 bp, homo sapiens
attactatttgcaaaaatgatgaatgtgtgctagaagataattctcagcggaccaaatggaaagtgatcagccccacagggaacgaggcaatggtgccgtcagtctgcttcctcatccccccacccaataaggatgccattgagatggccagcag

SEQ ID NO: 456 - DNA, 127 bp, homo sapiens
ggtcgaacaatcttatcagaaggttatggccctttggcatcagctgcatgttaacaccaaaagccttatctcttggaactatctgcgtaaagaccttgaccttgtacagacctggaacctagaaaag

SEQ ID NO: 457 - DNA, 84 bp, homo sapiens
ctatgtcaccttcacctgcgtcgggattgagcaagggtgagagagagagagaaatcaattccacgaattttggagaatgtccga

SEQ ID NO: 458 - DNA, 145 bp, homo sapiens
atgttggatgtttacagtctaatttcttttgacaatccagatggtggagtttggaagcaaggatttgacattacttatgaatctaatgaatgggacactgaaccccttcaagtctttgtggtgcctcattcccataacgacccag

SEQ ID NO: 459 - DNA, 34 bp, homo sapiens
atcagtacagcatccaggtggccttccacatcca

SEQ ID NO: 460 - DNA, 92 bp, homo sapiens
attccaactgatgcaccctgggaggctcaacatgctgacaaatgggacaaaatgaccatgaaagagctcattgacaaaatctgctggacaaa

SEQ ID NO: 461 - DNA, 112 bp, homo sapiens
ggtgattcgtcaacccgtgggcaggattttctttgcgggcacagagactgccacaaagtggagcggctacatggaaggggcagttgaggctggagaacgagcagctagggag

SEQ ID NO: 462 - DNA, 63 bp, homo sapiens
gtcttaaatggtctcgggaaggtgaccgagaaagatatctgggtacaagaacctgaatcaaag

SEQ ID NO: 463 - DNA, 58 bp, homo sapiens
gaagaagaaaatctgtgagctctatgccaaagtgctgggatcccaagaagctttacat

SEQ ID NO: 464 - DNA, 65 bp, homo sapiens
attcttcagcttgtctctaaccgaggaagcattgattgggagctactcattcagaaaattaaaag

SEQ ID NO: 465 - DNA, 291 bp, homo sapiens
ttgcaggagaaataacaaggcattgaagaatggcagatgaacggaaagatgaagcaaaggcacctcactggacctcagcaccgctaacagaggcatctgcacactcacatccacctgagattaaggatcaaggcggagcaggggaaggacttgtccgaagcgccaatggattcccatacagggaggatgaagagggtgcctttggagagcatgggtcacagggcacctattcaaataccaaagagaatgggatcaacggagagctgacctcagctgacagagaaacagcag

SEQ ID NO: 466 - DNA, 62 bp, homo sapiens
cagcaggtggggaatcagctctggctcccagtgtatttaaacaggcaaaggacaaagtctct

SEQ ID NO: 467 - DNA, 102 bp, homo sapiens
ggtcagagccaattcgcagagcagggaagagtggtacctcaacacccactacccctgggtctactgccatcactcctggcaccccaccaagttattcttcac

SEQ ID NO: 468 - DNA, 239 bp, homo sapiens
gcacaccaggcactcctggaacccctagctatcccaggacccctcacacaccaggaacccccaagtctgccatcttggtgccgagtgagaagaaggtcgccatcatacgtactcctccaaaatctcctgcgactcccaagcagcttcggcttattaaccaaccactgccagacctgaagaatgtcaaatccaaaatcggatcaacagacaacatcaaataccagcctaaaggggggcag

SEQ ID NO: 469 - DNA, 82 bp, homo sapiens
gtacaaattgttaccaagaaaatagacctaagccatgtgacatccaaatgtggctctctgaagaacatccgccacaggccag

SEQ ID NO: 470 - DNA, 110 bp, homo sapiens
gcggacgtgtgaaaattgagagtgtaaaactagatttcaaagaaaaggcccaagctaaagttggttctcttgataatgctcatcatgtacctggaggtggtaatgtcaag

SEQ ID NO: 471 - DNA, 282 bp, homo sapiens
attgacagccaaaagttgaacttcagagagcatgctaaagcccgtgtggaccatggggctgagatcattacacagtccccaggcagatccagcgtggcatcaccccgacgactcagcaatgtctcctcgtctggaagcatcaacctgctcgaatctcctcagcttgccactttggctgaggatgtcactgctgcactcgctaagcagggcttgtgaatatttctcatttagcattgaaataataatatttaggcatgagctcttggcaggagtgggctctga

SEQ ID NO: 472 - DNA, 78 bp, homo sapiens
cagctgaagaaacagctaatctgcctccttctccacccccatcacctgcctcagaacagactgtcacagtggaggaag

SEQ ID NO: 473 - DNA, 341 bp, homo sapiens
ggtcagagccaattcgcagagcagggaagagtggtacctcaacacccactacccctgggtctactgccatcactcctggcaccccaccaagttattcttcacgcacaccaggcactcctggaacccctagctatcccaggacccctcacacaccaggaacccccaagtctgccatcttggtgccgagtgagaagaaggtcgccatcatacgtactcctccaaaatctcctgcgactcccaagcagcttcggcttattaaccaaccactgccagacctgaagaatgtcaaatccaaaatcggatcaacagacaacatcaaataccagcctaaaggggggcag

SEQ ID NO: 474 - DNA, 477 bp, homo sapiens
ttgagctcttcccaaatggccaaagtgtcggggagaagattttcaagacagcaggctttgtaaaaccatttacggaggcacagctgctgtgcacacaggctggtggacagttggcctctccacgctctgccgctgagaatgccgccttgcaacagctggtcgtagctaagaacgaggctgctttcctgagcatgactgattccaagacagagggcaagttcacctaccccacaggagagtccctggtctattccaactgggccccaggggagcccaacgatgatggcgggtcagaggactgtgtggagatcttcaccaatggcaagtggaatgacagggcttgtggagaaaagcgtcttgtggtctgcgagttctgagccaactggggtgggtggggcagtgcttggcccaggagtttggccagaagtcaaggcttagaccctcatgctgccaatatcctaataaaaaggtgaccat

SEQ ID NO: 475 - DNA, 34 bp, homo sapiens
cgtgggaatcatgacagcccgacgtgccctccct

SEQ ID NO: 476 - DNA, 29 bp, homo sapiens
ttgatctgaagttaaacaaaaaaccacga

SEQ ID NO: 477 - DNA, 34 bp, homo sapiens
aataaaaaccggcacaggattgatctgaagttaa

SEQ ID NO: 478 - DNA, 34 bp, homo sapiens
gattgatctgaagttaaacaaaaaaccacgagct

SEQ ID NO: 479 - DNA, 111 bp, homo sapiens
gaggaagtattctgcttcatcaggtggtctatgcgaagaagcgacagctgctaaagtggcatcctgggattttgttgaagccacacaaagaacaaattgcagttgtttgag

SEQ ID NO: 480 - DNA, 684 bp, homo sapiens
gcacaaaaatctcaagtcaagaaatgctggacaacaaggacaggcaccatctttaggtcagcaacaacaaatacttcctaagcacaagaccaatgagaagcaagaaaagagtgaaaagccacagaaacgccccttgactccttttcaccatcgtgtgtctgttagtgatgatgttggcatggacgcagattcagccagccaaagacttgtgatctctgctccagacagtcaagtgagattttcaaatatccgaactaatgatgtagcaaagactcctcagatgcatggcaccgaaatggcaaattcacctcaaccacccccacttagtcctcacccttgtgatgtggttgatgaaggagtgactaaaacaccttcaactcctcagagtcaacatttttatcaaatgccaacaccagatcccttggttccttctaaaccaatggaagataggatagacagtttgtcccagtctttcccacctcaatatcaggaagctgtagaacctacagtatatgttggtacagcagtaaacttggaagaagatgaagccaatatagcctggaagtattacaagttcccaaagaaaaaagatgtagagtttttaccacctcaacttccaagtgataaattcaaggatgatccagttggaccttttggacaggaaagtgtaacatcagttacaga

SEQ ID NO: 481 - DNA, 101 bp, homo sapiens
gcctctttgcacctccacgtgccttcagtgcaatctgacgagctgcttcacagtaaacactcccacccacttgactcaaatcagacttcagatgtcctcag

SEQ ID NO: 482 - DNA, 195 bp, homo sapiens
gtttgttttggaacagtacaatgcactctcctggctaacctgtgaccctgcaacccaggacagacgctcatgtctcccaattcattttgtggtgctgaatcagttatataactttattatgaatatgctgtgatcttcatttgatggaactgtgcaagaaaagaacaaggaaaaatggatgtttcgctgcaggat

SEQ ID NO: 483 - DNA, 1210 bp, homo sapiens
acctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaatagg

SEQ ID NO: 484 - DNA, 192 bp, homo sapiens
acacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcag

SEQ ID NO: 485 - DNA, 130 bp, homo sapiens
ggtttctcgatcaggaccatcaacccctcatgtgaattttctcctggactcccatccagtgtctccagaagtgattgtggagcatacattaaaccaaaatggctacacactggttatcactgggaagaag

SEQ ID NO: 486 - DNA, 90 bp, homo sapiens
tgtggctggtgccacgacaaatgtgtgcgatcggaggaatgcctgagcgggacatggactcaacagatctgtctgcctgcaatctacaag

SEQ ID NO: 487 - DNA, 162 bp, homo sapiens
tgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattag

SEQ ID NO: 488 - DNA, 231 bp, homo sapiens
atcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagag

SEQ ID NO: 489 - DNA, 81 bp, homo sapiens
ggcattttggttgtgtatatcatgggactttgttggacaatgatggcaagaaaattcactgtgctgtgaaatccttgaaca

SEQ ID NO: 490 - DNA, 110 bp, homo sapiens
aatccaactgtaaaagatcttattggctttggtcttcaagtagccaaaggcatgaaatatcttgcaagcaaaaagtttgtccacagagacttggctgcaagaaactgtat

SEQ ID NO: 491 - DNA, 485 bp, homo sapiens
atatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctcttgccaaaattgcactattataggacttgtattgttatttaaattactggattctaaggaatttcttatctgacagagcatcagaaccagaggcttggtcccacaggccacggaccaatggcctgcagccgtgacaacactcctgtcatattggagt

SEQ ID NO: 492 - DNA, 34 bp, homo sapiens
accaaatcttttattagtggtgggagcacaataa

SEQ ID NO: 493 - DNA, 1214 bp, homo sapiens
ataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaatagg

SEQ ID NO: 494 - DNA, 707 bp, homo sapiens
aaaagtcattgaagagcttgtggggctgtgggacctgcgccctctggaggaattccatacacccactcaactctggcaaataggaaattgtcaagtaggagacaaggagcaaagtcctatcacagcgggaggggacgccagcgcctgcagaggctgagcagggaaaaagccagtgccccagcggaagcacagctcagagctggtctgccatggacatcctggtcccactcctgcagctgctggtgctgcttcttaccctgcccctgcacctcatggctctgctgggctgctggcagcccctgtgcaaaagctacttcccctacctgatggccgtgctgactcccaagagcaaccgcaagatggagagcaagaaacgggagctcttcagccagataaaggggcttacaggagcctccgggaaagtggccctactggagctgggctgcggaaccggagccaactttcagttctacccaccgggctgcagggtcacctgcctagacccaaatccccactttgagaagttcctgacaaagagcatggctgagaacaggcacctccaatatgagcggtttgtggtggctcctggagaggacatgagacagctggctgatggctccatggatgtggtggtctgcactctggtgctgtgctctgtgcagagcccaaggaaggtcctgcaggaggtccggagagtactgagaccg

SEQ ID NO: 495 - DNA, 28 bp, homo sapiens
ctggcagcagtaagatcttccctggcaa

SEQ ID NO: 496 - DNA, 500 bp, homo sapiens
cacccagcagcaatgacgataacccctggatccaggtgaacctgctgcggaggatgtgggtaacaggtgtggtgacgcagggtgccagccgcttggccagtcatgagtacctgaaggccttcaaggtggcctacagccttaatggacacgaattcgatttcatccatgatgttaataaaaaacacaaggagtttgtgggtaactggaacaaaaacgcggtgcatgtcaacctgtttgagacccctgtggaggctcagtacgtgagattgtaccccacgagctgccacacggcctgcactctgcgctttgagctactgggctgtgagctgaacggatgcgccaatcccctgggcctgaagaataacagcatccctgacaagcagatcacggcctccagcagctacaagacctggggcttgcatctcttcagctggaacccctcctatgcacggctggacaagcagggcaacttcaacgcctgggttgcggggagctacggt

SEQ ID NO: 497 - DNA, 106 bp, homo sapiens
gggcttgcatctcttcagctggaacccctcctatgcacggctggacaagcagggcaacttcaacgcctgggttgcggggagctacggtaacgatcagtggctgcag

SEQ ID NO: 498 - DNA, 45 bp, homo sapiens
ccagggggcccgtaactttggctctgtccagtttgtggcatccta

SEQ ID NO: 499 - DNA, 34 bp, homo sapiens
gtgacaggcatcatcacccagggggcccgtaact

SEQ ID NO: 500 - DNA, 142 bp, homo sapiens
ttccctggcaactgggacaaccactcccacaagaagaacttgtttgagacgcccatcctggctcgctatgtgcgcatcctgcctgtagcctggcacaaccgcatcgccctgcgcctggagctgctgggctgttagtggccac

SEQ ID NO: 501 - DNA, 96 bp, homo sapiens
ccacccccaggtcttcctgctttccatgggcccgctgcctcttggcttctcagcccctttaaatcaccatagggctggggactggggaaggggagg

SEQ ID NO: 502 - DNA, 521 bp, homo sapiens
gtgttcagaggcagcaccaccacacagtcacccctccctccctctttcccaccctccacctctcacgggccctgccccagcccctaagccccgtcccctaacccccagtcctcactgtcctgttttcttaggcactgagggatctgagtaggtctgggatggacaggaaagggcaaagtagggcgtgtggtttccctgcccctgtccggaccgccgatcccaggtgcgtgtgtctctgtctctcctagcccctctctcacacatcacattcccatggtggcctcaagaaaggcccggaagcgccaggctggagataacagcctcttgcccgtcggccctgcgtcggccctggggtaccatgtggccacaactgctgtggccccctgtccccaagacacttccccttgtctccctggttgcctctcttgccccttgtcctgaagcccagcgacacagaagggggtggggcgggtctatggggagaaagggagcgaggtcagaggagggcatgggttggcagg

SEQ ID NO: 503 - DNA, 85 bp, homo sapiens
gtgggcgtttggggccctctatgctggcttttcaccccagaggacacaggcagcttccaaaatatatttatcttcttcacgggaa

SEQ ID NO: 504 - DNA, 34 bp, homo sapiens
ggactggcagcagtaagatcttccctggcaactg

SEQ ID NO: 505 - DNA, 850 bp, homo sapiens
atcttccctggcaactgggacaaccactcccacaagaagaacttgtttgagacgcccatcctggctcgctatgtgcgcatcctgcctgtagcctggcacaaccgcatcgccctgcgcctggagctgctgggctgttagtggccacctgccacccccaggtcttcctgctttccatgggcccgctgcctcttggcttctcagcccctttaaatcaccatagggctggggactggggaaggggagggtgttcagaggcagcaccaccacacagtcacccctccctccctctttcccaccctccacctctcacgggccctgccccagcccctaagccccgtcccctaacccccagtcctcactgtcctgttttcttaggcactgagggatctgagtaggtctgggatggacaggaaagggcaaagtagggcgtgtggtttccctgcccctgtccggaccgccgatcccaggtgcgtgtgtctctgtctctcctagcccctctctcacacatcacattcccatggtggcctcaagaaaggcccggaagcgccaggctggagataacagcctcttgcccgtcggccctgcgtcggccctggggtaccatgtggccacaactgctgtggccccctgtccccaagacacttccccttgtctccctggttgcctctcttgccccttgtcctgaagcccagcgacacagaagggggtggggcgggtctatggggagaaagggagcgaggtcagaggagggcatgggttggcagggtgggcgtttggggccctctatgctggcttttcaccccagaggacacaggcagcttccaaaatatatttatcttcttcacgggaa

SEQ ID NO: 506 - DNA, 91 bp, homo sapiens
gtttttgccaatccagaagactgtgtagcatttggcaaaggagaaaatgccaagaagtatcttcgaacagatgacagagtagaacgtgtac

SEQ ID NO: 507 - DNA, 153 bp, homo sapiens
actgtaaaggatcagaaagtcattggcagagagaagcagcagtattaccctaggaataggagaaagtgggtcagcaactccctacaggcaagactgatggggagaagatgcatttcaagacgcgaaagaaaactgactctagatgtagagaag

SEQ ID NO: 508 - DNA, 298 bp, homo sapiens
gtggtgacagctgaccacatccaactcattgtgccccttgtgctggagcagaacctgtggtcatgtattcctggtgaagacaccatcatgaatgtccctggcttcttcctggtgcgtcgtgagaatccagagtactttcctcgtgggagcagttactgggaccgctgtgtagtagggggctacctctctccaaagacagtcgcagatacatttgagaaggtagtggctggctccatcaattggccagccatagggtccctcttggactatgtgatccgcccggccccacccccagaag

SEQ ID NO: 509 - DNA, 778 bp, homo sapiens
gtactgggtcacagggattcctcctttcccccccaaatattaactccagaaactaggcctgactggggacacctgagagtagtatagtagtgcaaaatggaagactgatttttgactctattataatcagcttcagagattccttaaaccttcctaatttcctgctccagggcagtaaacacaaatatttcttcaaggggtgatgaaaacctcggaagttttaatttgaggttatctgctacgaaacagtatttctaaaaggctaaagtgataagtctcttgcttttttttgatcctgctcttatattcttttttttcctcagagaaatcaggagggtagttagaggtataaaacaggaggaaatattatggaaaatgaaaatagggaaaataattgaatcattttagaagtagctaatttcttttctcaaaagagtgtcccttcttcacacctactcactttacaactttgctcctaactgtgggttgaaaactctagctaaagaaagttatcaaatcttaacatgcattcctactattatgatagtttttaaggtttcaattcaatcttctgaacggcataagtcctattttagccttacctcctgcatttgcaatacgtaatactgatcagtgggcacagttcttcagctacattgagaccctgaaatgaacaattatattctgactcgacatcttgtccccaatccttccaaaaatattgatggtgatttgtgctaccatttactcgtttatttaataaagacattcaatccca

SEQ ID NO: 510 - DNA, 34 bp, homo sapiens
agtagagttaagcccagatggttctgactctaga

SEQ ID NO: 511 - DNA, 206 bp, homo sapiens
ctgaagattcatttttgtctgccattataaattatactaatagctctacagtccactttaagttgtcccctacatatgtattatatatggcatgccggtatgtattgtccaaccagtacagacctgacatcagccctacagagcgcacacataaagtcattgcagtcgtcaacaagatggtgagcatgatggagggtgtcatccag

SEQ ID NO: 512 - DNA, 85 bp, homo sapiens
gaccaaactcttacccaggatctactggagcagctgttggagcccatgacgcctgtcgggatgcaaaagagaagcgctctaaaag

SEQ ID NO: 513 - DNA, 34 bp, homo sapiens
gagaagcgctctaaaagccaagatgcagattcac

SEQ ID NO: 514 - DNA, 34 bp, homo sapiens
aggtgctgcagatgatggatggacgtctagctgc

SEQ ID NO: 515 - DNA, 163 bp, homo sapiens
atctccttggccagcaggacgcccaaggaccagagcctgtgtcacgtcctggaacaggtgtgcggcgtcctcaagcaggggagctgagccttccagggcagggtgggctccagttgtcttgagggtccggatgggctcaggtaataaagaaacggaagcagca

SEQ ID NO: 516 - DNA, 167 bp, homo sapiens
ctctttggcctatagcagctgtggaaatttatacctcccgggtgctggaggctgttaatgggacagatgctcggttaaaatgcactttctccagctttgcccctgtgggtgatgctctaacagtgacctggaattttcgtcctctagacgggggacctgagcagttt

SEQ ID NO: 517 - DNA, 211 bp, homo sapiens
gtattctactaccacatagatcccttccaacccatgagtgggcggtttaaggaccgggtgtcttgggatgggaatcctgagcggtacgatgcctccatccttctctggaaactgcagttcgacgacaatgggacatacacctgccaggtgaagaacccacctgatgttgatggggtgataggggagatccggctcagcgtcgtgcacactg

SEQ ID NO: 518 - DNA, 56 bp, homo sapiens
tacgcttctctgagatccacttcctggctctggccattggctctgcctgtgcactg

SEQ ID NO: 519 - DNA, 92 bp, homo sapiens
atgatcataatagtaattgtagtggtcctcttccagcattaccggaaaaagcgatgggccgaaagagctcataaagtggtggagataaaatc

SEQ ID NO: 520 - DNA, 76 bp, homo sapiens
aaaagaagaggaaaggctcaaccaagagaaaaaggtctctgtttatttagaagacacagactaacaattttagatg

SEQ ID NO: 521 - DNA, 1818 bp, homo sapiens
gaagctgagatgatttccaagaacaagaaccctagtatttcttgaagttaatggaaacttttctttggcttttccagttgtgacccgttttccaaccagttctgcagcatattagattctagacaagcaacacccctctggagccagcacagtgctcctccatatcaccagtcatacacagcctcattattaaggtcttatttaatttcagagtgtaaattttttcaagtgctcattaggttttataaacaagaagctacatttttgcccttaagacactacttacagtgttatgacttgtatacacatatattggtatcaaaagggataaaagccaatttgtctgttacatttcctttcacgtatttcttttagcagcacttctgctactaaagttaatgtgtttactctctttccttcccacattctcaattaaaaggtgagctaagcctcctcggtgtttctgattaacagtaaatcctaaattcaaactgttaaatgacatttttatttttatgtctctccttaactatgagacacatcttgttttactgaatttctttcaatattccaggtgatagatttttgttgttttgttaattaatccaagatttacaatagcacaacgctaaatcacacagtaactacaaaaggttacatagatatgaaaagattggcagaggccattgcaggatgaatcacttgtcacttttcttctgtgctgggaaaaataatcaacaatgtgggtctttcatgagcagtgacggatagtttagcttactatgtttcccccccaattcaatgatctataacaacagagcaaagtctatgctcatttgcagactggaatcattaagtaatttaataaaaaaattgtgaaacagcatattacaagtttgaaaattcagggctggtgaaaaaaatcaactctaaatgatgataattttgtacagttttatataaaactctgagaactagaagaaattattaactttttttcttttttaattctaattcacttgtttattttgggggaggaagactttggtatggagcaaagaaataccaaaactactttaaatggaataaaaccaactttattctttttttcccccatactggtagataaagcaaactttataagtgggctattgaaagaaaagttacaagcttaagatacagaagcatttgttcaaaggatagaaagcatctaaaagtttaggctcaagatcaatctttacagattgatattttcagtttttaatcgactggactgcagatgttttttcttttaacaaactggaattttcaaacagattatctgtatttaaatgtatagaccttgatatttttccaatactattttttaaaaaattgtatgatttacatatgaacctcagttctgaaattcattacatatctgtctcattctgccttttatactgtctaaaaaagcaaagttttaaagtgcaattttaaaactgtaaattacatctgaaggctatatatcctttaatcacattttatattttttcttcacaattctaacctttgaaaatattataactggatatttcttcaaacagatgtcctggatgatggtccataagaataatgaagaagtagttaaaaatgtatggacagtttttccggcaaaatttgtagcttatgtcttggctaaatagtcaaggggtaatatgggcctgttgtttagtgtctccttcctaaagagcacttttgtattgtaatttattttttattatgctttaaacactatgtaaataaacctttagtaataaagaattatcagttatataa

SEQ ID NO: 522 - DNA, 34 bp, homo sapiens
actaacaattttagatggaagctgagatgatttc

SEQ ID NO: 523 - DNA, 325 bp, homo sapiens
gtaaggttcacaaataggttgatttctttcttcagctttctgacatgtccagcccatctctaatgaggactcccagatcatcactttatggctgttaggtgtttcccatatgaaattagaggagctgggtcagggagacaaaagtcttctattagtcttatggatagctcctccttgagtgtattttgtgcaaaagattaagaagctggactctactgccattaaagctgagagaatcctaaggttatttgtggcttcggggttatatttattactactactactaataaatattcaacaagtaaataaatcttttttaaatcaa

SEQ ID NO: 524 - DNA, 34 bp, homo sapiens
actaacaattttagatggtaaggttcacaaatag

SEQ ID NO: 525 - DNA, 148 bp, homo sapiens
tacgcttctctgagatccacttcctggctctggccattggctctgcctgtgcactgatgatcataatagtaattgtagtggtcctcttccagcattaccggaaaaagcgatgggccgaaagagctcataaagtggtggagataaaatc

SEQ ID NO: 526 - DNA, 237 bp, homo sapiens
ggggccccagaggagcaggagtctgggagggcccagagttcaccctctagtggatccaggaggagcagcacccgagccctggagtggcccagtacccttccaagaggccacagtcccagccaggacaaagtatgcggcccatcctggtgcgacagcgtgggacaatgtgaacatggactcgaagacatggccctttctctgtagttgattttttaaatgtgccattattgtttttaa

SEQ ID NO: 527 - DNA, 182 bp, homo sapiens
gacgaggcctttgccctgtggattgagcggtggggaaagctgtatgaggaggagtccccgtcccgcaccatcatccagtacatccacgacaactacttcctggtcaacctggtggacaatgacttcccactggacaactgcctctggcaggtggtggaagacacattggagcttctcaacag

SEQ ID NO: 528 - DNA, 118 bp, homo sapiens
gtggatgtgaaattggaccccaaggatttgcgaatagatacatttcgagccaaaggagcaggagggcagcatgttaataaaactgatagtgccgtcagacttgtccacatccccacag

SEQ ID NO: 529 - DNA, 77 bp, homo sapiens
gaaggatgttaacttggcggagttcgctgtggctgcaggggaccagatgctttacaggagtgaggacatccagctag

SEQ ID NO: 530 - DNA, 118 bp, homo sapiens
gggatgtcccactcttgttcaagcattgccaggccctagcacacaagtcactgcaggcagcaaccatacggcagtacttttaatggatggacaggtcttcacatttggaagtttttct

SEQ ID NO: 531 - DNA, 204 bp, homo sapiens
tgaaacactccataagaacaatgcccttaaagttgtgcgagagctacaagcccaaattgctgaacttcaggaagactttgaatccgagaaggcttcacggaacaaggccgaaaagcagaaaagggacttgagtgaggaactggaagctctgaaaacagagctggaggacacgctggacaccacggcagcccagcaggaactacg

SEQ ID NO: 532 - DNA, 351 bp, homo sapiens
caatacagtgggacagttaggaatgcacccggggcctcctgcagatttcggaaattggcaagctacgggattccttcctgaaagatcaactgtgtcttaaggctctccagcctatgcatactgtatcctgcttcagacttaggtacaattgctcccctttttatatatagacacacacaggacacatatattaaacagattgtttcatcattgcatctattttccatatagtcatcaagagaccattttataaaacatggtaagaccctttttaaaacaaactccaggcccttggttgcgggtcgctgggttattggggcagcgccgtggtcgtcactcagtcgctctgca

SEQ ID NO: 533 - DNA, 77 bp, homo sapiens
gtgtttcagctcaactcaccagtacacgtctgcggagaaacacatctgttggcaccccgttctggatggcccctgag

SEQ ID NO: 534 - DNA, 158 bp, homo sapiens
ggtacctctgaaagtggagcaagcaaacaatgctcgtgatgccctggcaaagacagtgtatagccatctttttgatcatgtggtaaacagagtaaatcagtgttttccttttgaaacatcatcctattttattggagtcctagatattgctggttttg

SEQ ID NO: 535 - DNA, 117 bp, homo sapiens
gagaaagacctttgaatctccgaatgcctaatttacagaacagacaaccccatcattttgtggtggatggggagctgagcagactttaccccagtgaggcaaagtcccactcatcag

SEQ ID NO: 536 - DNA, 865 bp, homo sapiens
agttcgaggaagggctgctggacagatgtcctgccccaggaccccatcccgcgctggtggagggtcgcaggagcagcgtgaaagtggaggctgaggccagccggcagtgagggttggactggtgtcttcagacccaggacctcagacttctggctcacacagacccccacgctctccatccccggaatctagtcacaaccctggatccttcctctgcccttctcctgcctccccacctgctccatgggcataagactgtggggcttcaagcaataacaagcagaggcctggagagaggacacaaggagggtgcgtggtgccctcacccctgcccagagcgagggggcaaggactctgcctccagggcatctggggttttcccctccctcacacaacacactcccattctctttaggtttgcaccagtggtgtgagcagttggactcagtttggacaagggggaaagggggacttccctgggaaggtccagctaaaagtggcaacatttgcccccagaattgggggcctgggaacactggacctgctccttctcccctctccttccccgtttttgtgcttctggtttgtttctttaattaatttaacaagtgctgcagtttgccctcccattcccatctatcccccaagtcctttgcaatttcttccctgccctacataggggcggtgggtgtgggatcccttcactggccccctcgggaggcctgggttggactcagggtctcctccagctgggggctggaccgcagcacctatctgagcagttagagcgtctttcttttcagattgtgtacagtagattatttattttgttattttggaataaaatttattttatggcttagga

SEQ ID NO: 537 - DNA, 155 bp, homo sapiens
gaattgcgccagtacctgtccaaccttgctgaccagtgcaacagtgagaacaatgctgtggacatgcccctcgtcatcatcctggacaacctacaccacgtgagctctctgggagagatcttcaatgggctgctcaactgcaagtaccacaaatg

SEQ ID NO: 538 - DNA, 116 bp, homo sapiens
gaggatatcattggccactcctcgacagctttataaatcttccaatatgactcagcgctggcaaagaagggaaatttcaaacttcgaatatttgatgttccttaatactattgcag

SEQ ID NO: 539 - DNA, 89 bp, homo sapiens
aatcgccactgggctcgtcacgacagcctccttcagctccaacccggctaattaatccccactcatggatataggcacacaaactaaag

SEQ ID NO: 540 - DNA, 34 bp, homo sapiens
aagcgcaagaggaagggcgctgccaagctgcagg

SEQ ID NO: 541 - DNA, 57 bp, homo sapiens
ctacctgggagccctccgtgaggccaagaatgacagcgagcagtttgcagccctgct

SEQ ID NO: 542 - DNA, 1269 bp, homo sapiens
accactgtccaaagagcctctccagagtcaagacccagaggcacactctctggcagcttcacaatgagcttcttctggtcaggtcgacagagacatctttgacgcaatctctgatgcttccagcaatcctcaaccttgtctgccctgccctaccccaactgtgtccacatccctgccgccaccccaccaaaaagctgcagaacattcttttgtcatctgatgaggtagagctatgttgggaatccaccaatgtgggcttggctttcccccacactgtagttagacagatagacagatagcccaggagccaggtgtcagggagcactgctgagagtatcacaataggatctgtcacggggttcatatcagatgaagcgccgtatccactgcttcacagagcaaaacattcaatcccataaccaggcacaggggaactaacttggactaactaaccagaaaaccttgttaacgtataacttgttccagtactacatctctgcctgctggctcatgacaattgctcagcacattttcccctcttgaagaaaggttgcaagaagaactaaattatcctcaaaagatttctgcttcattagtaaagagtcagtgatggaatagggtgactctgcagaatagtggcctctagggtaggagcttgttgtgttgtccgtgggcctggaatgatcctggtggctgatcagggtccttctcccactctgggctgtatcaaccctgacggtcttggtctttggctcccctttatctggattctgagcacgctgactgtcctgttaatgccttccctccaaggaccagtatttggagattaattagattacaactctatctatgttacctttgtccttcctggtcaccttgcagattcaagacatgttcaaagcaacacattcacaacccatttctattctatagcaacctcgtctgtgactccttagcctggagaacaatctaccaagaagagaaagtatctggaattaagaagtcctaccatccaagccctacttcctggttgtgtggccttggaaaagtgactcaacctctttatattcagtttcctaaccatgaagtggaaatgataacacctgcctcattggggcactataacaagtgaaggacttaggaaaacatctggagtatagcgcctggcacccaggagatgcttaataaatgggaaccaggattctttttcttttcttttttcttttctttttttttttttttgagacagagcctcaatctgtcacctgg

SEQ ID NO: 543 - DNA, 142 bp, homo sapiens
ctccttttcccagcagcagtttgcccaccaggggaatcctgcagtgtatagtatggtgcacatgaatggcagcagtggtcacatgggacagatgaacatgaaccccatgcccatgtctggcatgcctatgggtcctgatcag

SEQ ID NO: 544 - DNA, 34 bp, homo sapiens
ggtccggttcggcctggcaggtgttcgggacgcg

SEQ ID NO: 545 - DNA, 73 bp, homo sapiens
ggtataatgcaagatgtccaattacttgtcatgccccagggatttattgctcagtgcccagatcttaatcgca

SEQ ID NO: 546 - DNA, 168 bp, homo sapiens
tttacattcagcccatgatgggggccggcctgaattatgaatgttaccgattcggcatttccccatccacaaatgcgctggtgatcggtgccacggtgatggagggcttctacgtcatcttcgacagagcccagaagagggtgggcttcgcagcgagcccctgtgcag

SEQ ID NO: 547 - DNA, 221 bp, homo sapiens
cctggaactctagcacgccgagtgaacttgaatctttggctatttaaggaggactgggtttgttgtgaagttgcggtgatccagcgcagagccccgtcctgattgatcgcatcgcggggctcagatgactgtaaaatgaatagatgaaattcttgcttctcgaagattttcttgggcatctcccggaaagtgcgttttaaggcgaagtcatgatgtattct

SEQ ID NO: 548 - DNA, 34 bp, homo sapiens
tttggctatttaaggaggactgggtttgttgtga

SEQ ID NO: 549 - DNA, 73 bp, homo sapiens
aaaaaaaaaagtactaggtgcaaataacattatgaagtggtatttaattaaaaatccatgcagtgacagctga

SEQ ID NO: 550 - DNA, 251 bp, homo sapiens
gatctcctccctcacagcatcactacatgcacacagccgcaaaaaggtcaagaggaaacatccccgacgggcccaggactccaaaaagcccccaagggcccctcgtaccagcaaggcccagcgccgccgtctcacaggcaaagcacggcacagcggggagtgagaccgagaacgaagcggtgccccagtctaggctgcggggacctgtccttgctcagcttggctgtccctgtagcccagcctgcacctag

SEQ ID NO: 551 - DNA, 36 bp, homo sapiens
gaaccaacatgcctgcaaccttcggccatctggcag

SEQ ID NO: 552 - DNA, 137 bp, homo sapiens
gaaccaacatgcctgcaaccttcggccatctggcaggtggctacgacgcccagtactacgggtacctgtggagcgaggtgtattccatggacatgttccacacgcgcttcaagcaggagggtgtcctgaacagcaag

SEQ ID NO: 553 - DNA, 34 bp, homo sapiens
cggtcaggccagcctggatgcaccactgttggga

SEQ ID NO: 554 - DNA, 105 bp, homo sapiens
gttctgtggatggagttataaaggaagtgaatgtgagcccatgccccacccaaccctgccagctgagcaaaggacagtcttacagcgtcaatgtcaccttcacca

SEQ ID NO: 555 - DNA, 108 bp, homo sapiens
gttctgtggatggagttataaaggaagtgaatgtgagcccatgccccacccaaccctgccagctgagcaaaggacagtcttacagcgtcaatgtcaccttcaccagca

SEQ ID NO: 556 - DNA, 342 bp, homo sapiens
gtttctcatctctaagtgcctcattgagttcggtgcatctggccaatgagtctgctgagactcttgacagcacctccagctctgctgcttcaacaacagtgacttgctctccaatggtatccagtgattcgttgaagaggaggtgctctgtagcagaaactgagctccgggtggctggttctcagtggttgtctcatgtctctttttctgtcttaggtggtttcattaaatgcagcacttggttagcagatgtttaattttttttttaacaacattaacttgtggcctctttctacacctggaaatttactcttgaataaataaaaactcgtttgtcttgtc

SEQ ID NO: 557 - DNA, 282 bp, homo sapiens
acctcgaggcccccggggaggggcagggcctggagcttcccactaaaaacatgttttgatgctgtgtgcttttggctgggcctcgggctccaggccctgggaccccttgccagggagacccccgaacctttgtgccaggacacctcctggtcccctgcacctctcctgttcggtttagacccccaaactggagggggcatggagaaccgtagagcgcaggaacgggtgggtaattctagagacaaaagccaattaaagtccatttcagacctgcggcttctg

SEQ ID NO: 558 - DNA, 33 bp, homo sapiens
gcagaagagcactgatcgtactggctcactcag

SEQ ID NO: 559 - DNA, 102 bp, homo sapiens
agtaagaaggcagtgctttccatcaccactggtggcagtggctccatgtactctctgcaagggatccacggggacatgaatgtcattctctggccaattcag

SEQ ID NO: 560 - DNA, 123 bp, homo sapiens
gatcaccattgtggaccaaagaaaaacttgaaccaatcacacttcaaagtggtcagtctttagtacttccctgcagacccccaattggattaccaccacctataatattttggatggataatt

SEQ ID NO: 561 - DNA, 132 bp, homo sapiens
cggctccatactggatcacagcccctcaaaatcttgtgctgtccccaggagaggatgggaccttgatctgcagagctaatggcaaccccaaacccagaattagctggttaacaaatggagtcccaatagaaa

SEQ ID NO: 562 - DNA, 125 bp, homo sapiens
gttcactgttgacaaggatcatctagtggtagctgatgtcagtgacgatgacagcgggacctacacgtgtgtggccaacaccactctggacagcgtctccgccagcgctgtgcttagcgttgttg

SEQ ID NO: 563 - DNA, 123 bp, homo sapiens
tccccagtgctccctcgtctttgaagattgtgaatccaacactggactctctcactttggaatgggatccaccgagccacccgaatggcattttgacagagtacaccttaaagtatcagccaa

SEQ ID NO: 564 - DNA, 34 bp, homo sapiens
taatactccagaaggagtccccagtgctccctcg

SEQ ID NO: 565 - DNA, 177 bp, homo sapiens
ttaacagcacacatgaattaggccctctggtagatttgaaaattcctgccaacaagacacggtggactttaaaaaatttaaatttcagcactcgatataagttttatttctatgcacaaacatcagcaggatcaggaagtcaaattacagaggaagcagtaacaactgtggatgaag

SEQ ID NO: 566 - DNA, 34 bp, homo sapiens
cttaaagtatcagccaattaacagcacacatgaa

SEQ ID NO: 567 - DNA, 79 bp, homo sapiens
ttaaagaaaaggaagatgcccatgctgaccctgaaatccagcctatgaaggaagatgatgggacatttggagaatacag

SEQ ID NO: 568 - DNA, 34 bp, homo sapiens
ggaagtacctcttgatctggtacagcctccaacc

SEQ ID NO: 569 - DNA, 106 bp, homo sapiens
attaacaacaacctgagacacacggatgaaatgttctggaaccacgtatccatgtgcattttaaatggaggtggtatccggtcgcccattgatgaacgcaacaatg

SEQ ID NO: 570 - DNA, 146 bp, homo sapiens
gcgtcagccctcacgtcacttcgccagcagtagcagaggcggcggcggcggctcccggaattgggttggagcaggagcctcgctggctgcttcgctcgcgctctacgcgctcagtccccggcggtagcaggagcctggacccaggc

SEQ ID NO: 571 - DNA, 158 bp, homo sapiens
ctttctctcaggttctccagcttctcccatgggtgatatcctgcagaccccacagttccagatgagacggctgaagaagcagcttgctgatgagagaagtaatagggatgagctggagctggagctagctgagaaccgcaagctcctcaccgagaagg

SEQ ID NO: 572 - DNA, 73 bp, homo sapiens
ggatgagaaagatagtcaaggagaaaacatgtttctgcgctgtgtgagtgagcttgtgattgaaagccgagag

SEQ ID NO: 573 - DNA, 95 bp, homo sapiens
aggaagcggggggctagcggcctggcgctgcggcgaccgaccggggcgtcaggatccctggcccccggagagcgaagggcgggcgggtcccggag

SEQ ID NO: 574 - DNA, 71 bp, homo sapiens
ccctggggattttggatgctgatctggactcctccgtgcccgctgacattatctcatccacagacaaactt

SEQ ID NO: 575 - DNA, 136 bp, homo sapiens
tggccaacaagtggatacatgagtatggacaggaattccgcagaccctgcagctccagccctgaagactgaactgttggcagagagaagctggtggagtcctgtggctttccagagaagccaggagccaaaagctg

SEQ ID NO: 576 - DNA, 56 bp, homo sapiens
gagactccataaaggcatatggtgctgggctcctgtcatcctttggtgaattacag

SEQ ID NO: 577 - DNA, 68 bp, homo sapiens
acagcctttaattgctggcatgtaccttatgatgtctgttgacactgttatcccagcaggagaaaaag

SEQ ID NO: 578 - DNA, 158 bp, homo sapiens
gagcagaagaggaagagccgggggctgccgtagcctttggagatggacgagcagcccaggctgatgcattcccatgctggggtcgggatggccggacaccccggcctgtcccagcacttgcaggatggggccggagggaccgagggggagggcgggag

SEQ ID NO: 579 - DNA, 34 bp, homo sapiens
ccaacgtgcaatcacaggtggatacccttcgcca

SEQ ID NO: 580 - DNA, 75 bp, homo sapiens
gaagcaggacatcggcgacatcctccaccagatcatgaccatcaccgaccagagcttggacgaggcgcaagcaaa

SEQ ID NO: 581 - DNA, 34 bp, homo sapiens
gcggacggcgacggcaggaagcaggacatcggcg

SEQ ID NO: 582 - DNA, 74 bp, homo sapiens
gaaacatgccctgaactgtcacagaatgaaaccagcgctcttcagcgtcctgtgtgagatcaaagagaaaacag

SEQ ID NO: 583 - DNA, 34 bp, homo sapiens
gacgaggcgcaagcaaagaaacatgccctgaact

SEQ ID NO: 584 - DNA, 102 bp, homo sapiens
gtctcagcatcagaggagcccaggaggaggaccctcccgatccccagctaatgagactggacaatatgcttttggcagaaggggtttcaggtcctgagaaag

SEQ ID NO: 585 - DNA, 113 bp, homo sapiens
ccgcggcagcctctggaggttcttcagataactctattgaacactcagattacagagccaaattgacccagatcagacaaatctatcacacagaactggagaaatatgaacag

SEQ ID NO: 586 - DNA, 34 bp, homo sapiens
ggcggcagcagctgcagccgcggcagcctctgga

SEQ ID NO: 587 - DNA, 34 bp, homo sapiens
gatcaaagagaaaacaggtctcagcatcagagga

SEQ ID NO: 588 - DNA, 115 bp, homo sapiens
gcatgtaatgaatttactacacatgtgatgaaccttctccgagaacagagtagaacacgtcccatttctccaaaagagattgaaagaatggtgggcatcatccatcgaaaattta

SEQ ID NO: 589 - DNA, 76 bp, homo sapiens
gttccattcagatgcagctcaaacaaagcacttgtgaagcagttatgattttaagatcaaggttccttgatgccag

SEQ ID NO: 590 - DNA, 34 bp, homo sapiens
tggagaaatatgaacaggcatgtaatgaatttac

SEQ ID NO: 591 - DNA, 136 bp, homo sapiens
acggaaaaggcgtaacttcagtaaacaggccacagaaatcttgaatgaatatttttactcacacctcagcaacccctaccccagtgaagaagccaaagaggagctggccaagaaatgcagcatcacagtgtcacag

SEQ ID NO: 592 - DNA, 161 bp, homo sapiens
gtatccaattggtttggcaacaaacgaatcaggtacaagaagaacattggcaagtttcaggaagaagccaacctctatgctgcaaagacggccgtgacagctgcacacgcagtagcagcagctgtgcagaacaaccagaccaattcgcccaccacaccaaa

SEQ ID NO: 593 - DNA, 112 bp, homo sapiens
agtcggagccaatgtgcaatcacaggttcttctggttcttttaacctcccaaattctggggacatgttcatgaacatgcagagtctgaatggggattcttaccaagggtccc

SEQ ID NO: 594 - DNA, 451 bp, homo sapiens
gctaatggaggctggcaggacgcaacaactccatcttctgtgacttctcctacagaaggcccaggaagtgtgcactcggatacctctaactaatctctggccacacttttccctgagctacatgccttgataagtgcattcagagcaataggaggaaaaggaaagcgtttttgtagcccaccatctacagctttactgtaaaaccttgtcttattcgagaacttggtaaatctgttttttaaggaatcataatcatttgtatttatacttaaaaacacacaatgttaaaaaaaataaagcactttatccaattaggccaagatttaacattgttgacagtcctgtagctattttatcataatttattatcaatattttacattaatggtttcacagttgccaattacttggccttaagggtaaaaagtacaatatacactaaacctcaacc

SEQ ID NO: 595 - DNA, 809 bp, homo sapiens
gttaaagcagatgcaaaaattcacctcacctaaattgaacttcttgcatatttccattactgacttggattgtctttctttcatatcactaatggagttggaataaagagctgtttgcctatccctgttaatgatggttgtgtttaagaatcttcctcgtcacgtttgtgttcagatctcttatgttataattagatcagagactggtagcatcgtttctctctctgaaagcaccagtgcccagagtctgctcggtaataaaattatggatccagattgttctgagagacgaagatacttgctgctgatagaggtgaaaacgagattgatccgtctggggttttacggtgtgcactgggtgctgcacagacttgtcaaggtttgctacgtcctctgggcatctgcaaaaggccctgctctctggagtgttgtatatagtgtagcaaaagagtatttatacatcccaccaatcaaaacacagctttattacctcatgcgaactcatacaaaccaatagaatttcaacatgttctgtagcttagagtgctcacttactacctctgaacaatactcacgctgtagtttgtctctttcttatctttttgcatcttgtaattaactctttgtttcccttcataaaatgtaatgtacattgtaatcttttaaaagaaaaatcagggttgcacttgcaacttttaaaaaaccgagtgtggaaacattgggtcttaattcaacacaggatcggtaaaactgttgtaaatactgagaaacattttgaatgttcttcatcttattactaatccatgcaa

SEQ ID NO: 596 - DNA, 267 bp, homo sapiens
ccgccgccgccgccgccgcctcagccttcgcctcagccgccgcccgctcccgcccgcgcgcggcgggatggacgatcaatccaggatgctgcagactctggccggggtgaacctggctggccactcggtgcaggggggcatggccctgccgcctcccccgcacggccacgaaggggcggacggcgacggcaggaagcaggacatcggcgacatcctccaccagatcatgaccatcaccgaccagagcttggacgaggcgcaagcaaa

SEQ ID NO: 597 - DNA, 242 bp, homo sapiens
gtctcagcatcagaggagcccaggaggaggaccctcccgatccccagctaatgagactggacaatatgcttttggcagaaggggtttcaggtcctgagaaaggtgggggatcggcggcagcagctgcagccgcggcagcctctggaggttcttcagataactctattgaacactcagattacagagccaaattgacccagatcagacaaatctatcacacagaactggagaaatatgaacag

SEQ ID NO: 598 - DNA, 191 bp, homo sapiens
gcatgtaatgaatttactacacatgtgatgaaccttctccgagaacagagtagaacacgtcccatttctccaaaagagattgaaagaatggtgggcatcatccatcgaaaatttagttccattcagatgcagctcaaacaaagcacttgtgaagcagttatgattttaagatcaaggttccttgatgccag

SEQ ID NO: 599 - DNA, 166 bp, homo sapiens
gtatccaattggtttggcaacaaacgaatcaggtacaagaagaacattggcaagtttcaggaagaagccaacctctatgctgcaaagacggccgtgacagctgcacacgcagtagcagcagctgtgcagaacaaccagaccaattcgcccaccacaccaaattccg

SEQ ID NO: 600 - DNA, 1260 bp, homo sapiens
gctaatggaggctggcaggacgcaacaactccatcttctgtgacttctcctacagaaggcccaggaagtgtgcactcggatacctctaactaatctctggccacacttttccctgagctacatgccttgataagtgcattcagagcaataggaggaaaaggaaagcgtttttgtagcccaccatctacagctttactgtaaaaccttgtcttattcgagaacttggtaaatctgttttttaaggaatcataatcatttgtatttatacttaaaaacacacaatgttaaaaaaaataaagcactttatccaattaggccaagatttaacattgttgacagtcctgtagctattttatcataatttattatcaatattttacattaatggtttcacagttgccaattacttggccttaagggtaaaaagtacaatatacactaaacctcaaccgttaaagcagatgcaaaaattcacctcacctaaattgaacttcttgcatatttccattactgacttggattgtctttctttcatatcactaatggagttggaataaagagctgtttgcctatccctgttaatgatggttgtgtttaagaatcttcctcgtcacgtttgtgttcagatctcttatgttataattagatcagagactggtagcatcgtttctctctctgaaagcaccagtgcccagagtctgctcggtaataaaattatggatccagattgttctgagagacgaagatacttgctgctgatagaggtgaaaacgagattgatccgtctggggttttacggtgtgcactgggtgctgcacagacttgtcaaggtttgctacgtcctctgggcatctgcaaaaggccctgctctctggagtgttgtatatagtgtagcaaaagagtatttatacatcccaccaatcaaaacacagctttattacctcatgcgaactcatacaaaccaatagaatttcaacatgttctgtagcttagagtgctcacttactacctctgaacaatactcacgctgtagtttgtctctttcttatctttttgcatcttgtaattaactctttgtttcccttcataaaatgtaatgtacattgtaatcttttaaaagaaaaatcagggttgcacttgcaacttttaaaaaaccgagtgtggaaacattgggtcttaattcaacacaggatcggtaaaactgttgtaaatactgagaaacattttgaatgttcttcatcttattactaatccatgcaa

SEQ ID NO: 601 - DNA, 120 bp, homo sapiens
catggccgccccgccgcgccccgcgccatcgccccccgccccgcggcgcctcgacacgagcgacgtcctgcagcagatcatggccatcaccgaccagagcctggacgaggcacaggccag

SEQ ID NO: 602 - DNA, 146 bp, homo sapiens
catgtgagtcaggatctggaagcctcgtcatgttcttcaacacaaggaaaatttaaccgagagcagttttacaaatttatcattttccctggcaagtggattaaagtctggtatgatcgactgaccttgctggcattacttgatcg

SEQ ID NO: 603 - DNA, 93 bp, homo sapiens
gggccaaagccaggcaccctcccagttacgttgtgtatggcctgaagctcttctctccagtgtttctacaatcagctagggactacttaatag

SEQ ID NO: 604 - DNA, 110 bp, homo sapiens
gaaccgtggagcatgcaacacatcccggcactgttttcggccttctgtggcctcttggtcgccctttcttaccatctgagccgtcagagcagtgacccatctgtactcat

SEQ ID NO: 605 - DNA, 93 bp, homo sapiens
acggatgtcttaaaatgggatctcatcgtctgcgcagtggttgctgtcctctcatttgcagtcagcgccagcactgtattcctgtcattgcga

SEQ ID NO: 606 - DNA, 142 bp, homo sapiens
atgttgcccatttaatgtggttcgaaagactctatgtttggcttcagtgttttgaaaaatacatcttgtacccagcgctaattttgaatgccctcactattgatgcatttttaataagcaatcaccggagacttggtaccca

SEQ ID NO: 607 - DNA, 342 bp, homo sapiens
cacctctccgagccatgtgagccccctgatgccaccgagcaggggcagcttcatgaccgatgtctggctgaggctgtggcggacactctcggggttgtctgcaggagagcaagccaggaggacatgggcctggacgacacggcctcgcagcaaagtgtgtcagacgagcagtgacgggcgtgcggccgggcggggaggctggctcccccacacctcccacctgcattgctctccctcgtgctccccaaatcaccacaaccaaccaataccgcgatccatgagggactcctcctgtggaaaaggagagctgttccagaacacagaactgatctcaggtttttg

SEQ ID NO: 608 - DNA, 360 bp, homo sapiens
gtacttgctgggacggattcttgcgggaagcgcggactccgagggggtggcagccccgcgccgcctccgccgtgccgccgaccacgatgtgggctctgagctgccccctgagggcgtgctgggggcgctgctgcgtgtgaaacgcctagagaccccggcgccccaggtgcctgcacgccgcctcttgccaccctgagcactgcccggatcccgtgcaccctgggacccagaagtgcccccgccatcccgccaccaggactgctccccgccagcacgtccagagcaacttaccccggccagccagccctctcacccgaggatccctaccccctggccccacaataaacatgatctgaagca

SEQ ID NO: 609 - DNA, 130 bp, homo sapiens
gggcaggagctgatgggtgcattaactgcacagagggctacttcatggaggatgggagatgcgtgcagagctgtagtatcagctattactttgaccactcttcagagaatggatacaaatcctgcaaaaa

SEQ ID NO: 610 - DNA, 241 bp, homo sapiens
cgaggccgagtcctgttcttctgagggacggaccccagctggggtggaaaagcagtaccagagagcctccgaggcgcgcggtgccaaccatggagcgggccggccccagcttcgggcagcagcgacagcagcagcagccccagcagcagaagcagcagcagagggatcaggactcggtcgaagcatggctggacgatcactgggactttaccttctcatactttgttagaaaagccaccag

SEQ ID NO: 611 - DNA, 90 bp, homo sapiens
gactttgctgcttatttggcattttgtggtattgttcttcataatgctcagctctatgagacttcactgctggagaacaagagaaatcag

SEQ ID NO: 612 - DNA, 138 bp, homo sapiens
gtgctgcttgaccttgctagtttaatttttgaagaacaacaatcattagaagtaattttgaagaaaatagctgccactattatctctttcatgcaagtgcagaaatgcaccattttcatagtggatgaagattgctcc

SEQ ID NO: 613 - DNA, 95 bp, homo sapiens
aacaagctgactgacctggagatacttgcattgctgattgctgcactaagccacgatttggatcaccgtggtgtgaataactcttacatacagcg

SEQ ID NO: 614 - DNA, 64 bp, homo sapiens
ggcaatgctgatgacagcttgtgatctttctgcaattacaaaaccctggcctattcaacaacgg

SEQ ID NO: 615 - DNA, 1415 bp, homo sapiens
gccctgacccacgtgtcagaggactgtttccctttgctagatggctgcagaaagaacaggcagaaatggcaggcccttgcagaacagcaggagaagatgctgattaatggggaaagcggccaggccaagcggaactgagtggcctatttcatgcagagttgaagtttacagagatggtgtgttctgcaatatgcctagtttcttacacactgtctgtatagtgtctgtatttggtatatactttgccactgctgtatttttatttttgcacaacttttgagagtatagcatgaatgtttttagaggactattacatattttttgtatatttgttttatgctactgaactgaaaggatcaacaacatccactgttagcacattgataaaagcattgtttgtgatatttcgtgtactgcaaagtgtatgcagtattcttgcactgaggtttttttgcttggggattattttaaataattggtttttgtgttttctgaattaccattttttcaagaatgtttggaatctttcctttttcaaaagtaggttaggagcaaattatcatacattctgtgacatttaaagcctttataggatagtgaaaaatgctggctgagtggattttaagagaaataattgtatttgttaacagtgtctttttttaaaaagttaaggcactctgaaacaaatggaaagtcctatgaaactgtattgtaaagaaaacattatttaattgatatgctgttttgtgagagaacaggcaagacagaactttgtcacttcagtgcagtacatttttctgaaagctacccataaaatcactttcatctcacctacctgatgcaaagcaggtgaaaccttaggagatgatccagtcactgacttgattgagggataagtgtgatttagaaatggaatggccttggatgtctatcagtgaagaaaaatgttctgttagaagatctctctaagagtttttttccttctgagcttccttttcaaaataaaagtgacaattgtagcattgacttgaagtgagacatggttatagataagagagtacaaaatgactctttttcctgtcaattgaaatttaaagaaaagttttaattatataaatagcaaagggctattgccaatactagggtcaaaaatgaatttgagggaacagtgggtaagaaactttatgcctgaataacatttagcagtattgtgattgaaaaattgccatattttgatgtataggacaagtcaactgagatccagagaatcctggatgtgaatgctaaacactggcccttaactcacattcaatgtattttcttcccataacatttagtatagttaatattttcttagaatttgagcccatttaagtggattaatattctacatgtgtgcccctaaagacacattt

SEQ ID NO: 616 - DNA, 533 bp, homo sapiens
cttctgctctactacagagagtgaagaagaaagtattaaagctcactttaccattccatatacttactaaaagcctgtgtaaacatgtcttaatgaatgttgttgaaagcaatgtaaatagttgaaaatataaatttatattacagtttaagaaaaccttatgaggcatcactagccactgttaatatctatttgtattcttataccttttcaatatatttgaacaaatatagtttctggcactatttttatactaggaaaaggagttactatgtatattatgcttagcttttaaggcattttaaataaccatgaatgttgatttcattactttcctttcctccatcacgagagtcatttcagatgactctttcatgacaaaatcactttaaaggaacacttacctccgattcctgtataaagtcatgagatggtcaaggtggttttccattgtgcaaattcttcacctgtcagtggtttcctcattttgccatgctttgtaaaaataaaaagaatgatcaagtaggtatgaa

SEQ ID NO: 617 - DNA, 34 bp, homo sapiens
aaggtggtcagtccctggtgtagctgcccagcac

SEQ ID NO: 618 - DNA, 167 bp, homo sapiens
gagcgctgcccgcgcggggaccacaaccaaagtcgcggccgccgcagccatgcgctgggtgtgggcgctgctgaagaatgcgtccctggcaggggcgcccaagtacatagagcacttcagcaagttctccccgtccccgctgtccatgaagcagtttctggacttcg

SEQ ID NO: 619 - DNA, 72 bp, homo sapiens
gtatatacagagcctgatggatttggtggaattccatgagaaaagcccagatgaccagaaagcattatcaga

SEQ ID NO: 620 - DNA, 185 bp, homo sapiens
ctttgtagatacactcatcaaagttcgaaatagacaccataatgtagtccctacaatggcacaaggaatcatagagtataaagatgcctgtacagttgacccagtcaccaatcaaaatcttcaatatttcttggatcgattttacatgaaccgtatttctactcggatgctgatgaaccagcaca

SEQ ID NO: 621 - DNA, 78 bp, homo sapiens
atgcctttgagtgttcaaggatgctctgtgatcagtattatttatcatctccagaattaaagcttacacaagtgaatg

SEQ ID NO: 622 - DNA, 99 bp, homo sapiens
aatgcaatgcgggcaacagttgaacaccaggaaaatcagccttcccttacaccaatagaggttattgttgtcttgggaaaagaagaccttaccattaag

SEQ ID NO: 623 - DNA, 111 bp, homo sapiens
atttcagacagaggaggtggtgttcccctgagaattattgaccgcctctttagttatacatactccactgcaccaacgcctgtgatggataattcccggaatgctcctttg

SEQ ID NO: 624 - DNA, 114 bp, homo sapiens
gctggttttggttacggcttgccaatttctcgtctgtatgcaaagtactttcaaggagatctgaatctctactctttatcaggatatggaacagatgctatcatctacttaaag

SEQ ID NO: 625 - DNA, 2281 bp, homo sapiens
gctttgtcttctgagtctatagaaaaacttccagtttttaacaagtcagccttcaaacattatcagatgagctctgaggctgatgactggtgtatcccaagcagggaaccaaagaacctggcaaaagaagtggccatgtgaagagggacactcaggacactttacgggatcaaagtgggtctacaccagtgctgcttcctgaatgtttgtgtgtgaacccttgtttcctccaaaacaaacgacagcaacgaaaactccttaatcagaacactgatccaatgaggaatggagcttgtttctgtgacccaggagaacttagtgcaagactacaggagttaacagatggccagctccttattttttaatgtagaataactcctgagtttatatcaaatcctgaagaaataagcctcagttttccatctgtttttgataagaataagaaagggagtgagtgtgaagatggtggttagcagtttcactaagactgatattttaggcctcttgttcacatcaaaagatattggtgtcagaataccagcattttcctgccatgcaaaggattaaaacttagtttacactatgtggttacaaatatatgtcaatgtacattttgaacatatttatgtgctatggaaggaaatgctggtgactaaaataaggtttactctgaaagaggaggaattttattcaaagcattcaaacattttattcaagtgtttcaaaattcaaagcattgtattcaaagttgcagtgaaggcatcaacttatgtaaaaactcagaaggaaggctcctctgataaaaacacagctcctttattatgctgcttttcttgttcactttacacactaagtaaacacttattgtcaggtgcctagtcttgagtgaattgttagatgtgcactgaactcgggatgttggggattggagagagagaattgccaaagtaacagcaaaaatatctcttactttgctttgtttataaataaattagtagattggaaaaactagtgttagggaaagaaatcacatgttcagagcctaattcagtaggaagggcttttctctaccctgaaatgaaggtaatccaaaggcatccattttctaggcttaaaagatatatttttgatatatttaattatattctctacactccagcattaatatgtctgtttaaaaattactaattctcaaatggctcaagaacattagaatttaagtaccttttagagtaattattttaagcaaatagcctggacgtaagagattctcatgccagcatgctttcatttgtcagttgttgtgactgagagataatgaatgacacctgaaatgcatatggtatttttgggagagttaaggtataatttgaaggttggcagaccagttgcgctgattactcttagagaagaagaaatggaaaaatgaaagaaggcaggaaggaaagaaaggatataggaagagagggaagcagaaggcaggcatttttctattttccccacaaattatttcaaaaaaaatctgtattttctgggatatgtcattggcaagaggaagaactggtgttttgaaagcagtatggattctttaaatgcctctcactcttacaagatagtaggctttgagataataaacttacccgtgtcaattaacatttaaactggcatatagaaaaaaaggaggatttttctgcattgtaaaataatcagtatggtttatatgttgaatttgacatttgtgtgtaatttcatggtggcctagtgttgtggtgcttctggtaatggtaatagaagctcaactatttttttgtggatttcagtttttatcatcagaagtcctagacagtgacatttcttaatggtgggagtccagctcatgcatttctgattatacaaaacagtttgcagtaggttatttgtcatttcagttttttactgaaatttgagctaaacatttttacatgtaaatacttgtatttaccaaagatttaaatcagttgattaattaattaactcaaatactgtgaactatctctaaaacactagaaaaaagaaatgttagtatctcaattacaccaactgtgcaaatgaactttgataaaatagaaataatctacattggcctttgtgaaatctggggaagagctttaggattctagtagatggatactgaatactcaggcccacttaaattattaatgtatacattgtgtttttgtctttatgctatgtacagagaaatgtgataattttttataataaatattttttatgatgataa

SEQ ID NO: 626 - DNA, 130 bp, homo sapiens
gaaataatggtatatctagccatgtatatgcgaacccagcctggcctctttgctgaaatgtttcgacttcgaattggtctgatcatacaagttatggcaacagaactggcccactcccttcgatgctcag

SEQ ID NO: 627 - DNA, 245 bp, homo sapiens
acgctggacgagtcggaccgaggctaggacgtggccggcgctctccagccctgcagcagaagaacttcccgtgcgcgcggatcctcgctccgttgcacgggcgccttaagttattggactatctaatatctatgtatttatttcgctggttctttgtagtcacatattttatagtcttaatatcttgtttttgcatcactgtgcccattgcaaataaatcacttggccagtttgcttttctacca

SEQ ID NO: 628 - DNA, 251 bp, homo sapiens
ggccatacgctggacgagtcggaccgaggctaggacgtggccggcgctctccagccctgcagcagaagaacttcccgtgcgcgcggatcctcgctccgttgcacgggcgccttaagttattggactatctaatatctatgtatttatttcgctggttctttgtagtcacatattttatagtcttaatatcttgtttttgcatcactgtgcccattgcaaataaatcacttggccagtttgcttttctacca

SEQ ID NO: 629 - DNA, 34 bp, homo sapiens
ggaggagctgaggttcggttcggtccccgccggg

SEQ ID NO: 630 - DNA, 148 bp, homo sapiens
agtcccaggagccgagctccagcactagagccagctgcgagcggagggcaccaactccgcagaactggcttttcaatgggacacctgtggctcctgggtatttggggcctctgtgggctgctcctgtgtgccgcggatcccagcacag

SEQ ID NO: 631 - DNA, 90 bp, homo sapiens
atggctctcaaataatccccaaagtcacagaaataatacctaaatatggcagtataaatggagcaacaaggctgactataagaggggaag

SEQ ID NO: 632 - DNA, 109 bp, homo sapiens
agcaatgccggaagattcctacactgttagagtcagtgtggacggggttcctgttacggaaaataacacctgcaaaggtcacatcaacagctgggaatgtaccttcaac

SEQ ID NO: 633 - DNA, 58 bp, homo sapiens
gcaaaaagttttagaaccccaacaataagaagcatcacacctttatctggaactccag

SEQ ID NO: 634 - DNA, 54 bp, homo sapiens
agtttacattggaggaatgccctgtgagcttctcataccacaatctgataattt

SEQ ID NO: 635 - DNA, 111 bp, homo sapiens
aggtcaccatgattttcccttcacaaggaagcattcgaggtggcaccacgctgacaataagtgggcgtttctttgatcagacagatttccccgtcagagttctagttggag

SEQ ID NO: 636 - DNA, 90 bp, homo sapiens
gtgaaccttgtgatattttgaatgtcacagaaaatagtatatgttgcaagacaccccccaaacctcatattctcaaaactgtatatccag

SEQ ID NO: 637 - DNA, 269 bp, homo sapiens
gagggagaggcctgaagcttgaggtgtggaataatagccgtccaatacgtttggaagagatactggaatacaatgaaaaaacgcctgggtacatgggtgccagttgggtagattcagcttcctatatttggctcatggaacaagacacatttgttgcacgctttagtggatttttggtggctccagattctgatgtttatagattctacatcaagggtgatgaccgttatgctatttattttagccagactggacttccagaagataag

SEQ ID NO: 638 - DNA, 136 bp, homo sapiens
gttataacattggaaaactgggaaacaactaatgcaattaatgaggttcagaagatcaaggtaaccagcccatgtgtggaagctaattcatgttcactttaccaatatagattaatctataatatggaaaaaactg

SEQ ID NO: 639 - DNA, 158 bp, homo sapiens
gagactttgatctgcttggttatgaagtagttgaagggaataatgtcacactggatattacagaacaaaccaaaggaaaacccaacttggagacattcacactgaattgggatgggatcgcttctaagccactcactctatggtcatcagaagctgaa

SEQ ID NO: 640 - DNA, 150 bp, homo sapiens
gaacatattaacagagggcagaagacagctgaaaccgatgcttactgtggtcgttattccctgaaaaacccagctgttctttttgactcagcagatgttaaaccaaacagacgaccatatggagatattttattgtttccttataatcag

SEQ ID NO: 641 - DNA, 125 bp, homo sapiens
ttatgtttagcatacaaaggattcctggcaaattatattggtctaaaatttcagtaccaagacaatagcaagattactagaagcactgatacacagtttacatacaactttgcttatggaaacaa

SEQ ID NO: 642 - DNA, 133 bp, homo sapiens
aaaactgagggtacagttgatatttcagttactaccaatggatttcaagccacagcaagggatgcttttagttataattgtttacagacaccaattataactgattttagtccaaaagtacgaacaatactag

SEQ ID NO: 643 - DNA, 196 bp, homo sapiens
gagaagttaatttaacaattaagggctataattttggaaatgaactcacacaaaacatggcggtgtatgttggaggaaaaacctgccagattcttcactggaacttcacagatattagatgccttttgcccaagttgtctcctggaaaacatgatatctatgtagaagtcagaaactggggttttgcatcaacaag

SEQ ID NO: 644 - DNA, 171 bp, homo sapiens
gttcattttcttaccaatttacttctcctggaatccattattatagcagcgggtatgttgatgaggctcactccatttttctccaaggagtcattaatgttttaccagctgaaaccagacacattcccttgcacctgtttgtgggtcgctctgaagccacatatgcttatg

SEQ ID NO: 645 - DNA, 985 bp, homo sapiens
gttacaattggtagctacccctgtgtcgtagaagaaagtagtgaggattcaattacatgtcatattgaccctcaaaactcaatggatgttggtatcagggaaactgtcactttgactgtctacaacctgggcactgctatcaatacgttgtccaatgaatttgataggcgatttgtacttttgccaaacattgacctggtgttgccaaatgcaggatcaactacaggaatgacaagcgtgaccataaaaggctctggatttgccgtttcttctgcaggtgtaaaagtccttatgggtcatttcccatgtaaagttctatcagtgaattatacggccattgaatgtgaaacatcccctgctgcccaacagcttgtggatgtagatcttctaatacatggagtgcctgcccagtgccagggaaactgcaccttttcatacttagaaagcatcactccttacataacaggagtcttcccaaactctgtcataggatctgtaaaagttcttattgaaggagaaggtttggggactgttttggaggacattgctgttttcattggaaatcaacagttcagagcaatagaggttaatgaaaacaacatcactgctcttgtgactcctctcccagttggacatcattctgttagtgttgtggtgggaagtaaaggcttggctctgggaaacctgactgtcagcagccccccagtagcatctctatcaccaacttctggaagcattggtggtggaactacactggtgatcacaggaaatggcttctatccaggcaacactacagtcactattggggatgaaccttgtcaaattatttccatcaaccccaatgaagtctactgccgcactcccgctgggaccactggaatggtcgatgttaaaatctttgttaatacaattgcttatccacctttgctttttacatatgccctggaggatactccatttctcagaggaattatcccaagcagag

SEQ ID NO: 646 - DNA, 157 bp, homo sapiens
tgaaaatatggaggatgttcatatcaccatagctgaagccaaatgtgatgttgagtattccaacaagacacacatcatctgcatgacagatgcccatactctatcagggtgggctccagtttgtgtccacatcagaggtgtcggcatggccaaactg

SEQ ID NO: 647 - DNA, 157 bp, homo sapiens
gataatgctgactttctttatgttgatgcctggtcctccaatttctcatgggggggaaaatctcccccagaagaaggatctcttgttgttattacaaaaggacagaccattctgctggatcaaagcacccctattttgaaaatgttgcttattcagg

SEQ ID NO: 648 - DNA, 130 bp, homo sapiens
attggaacagagacatccccattccaacacaaggctgtcattaccttgcatggtcacctgcgatctcctgagctccctgtctatggtgccaaaacactggctgtgcgggagggaatcctggatctgcacg

SEQ ID NO: 649 - DNA, 130 bp, homo sapiens
gtgtgcctgttcctgtgacctggactcgcttggctcatactgcaaaggcaggggaaagaattttaattttacaagaagcagtaacatggaaaccaggagataacattgtaattgcaagcacaggacacag

SEQ ID NO: 650 - DNA, 242 bp, homo sapiens
acacagtcaaggagagaatgaaaaaatgaccattgcatctgtgtctgctgatggcataaacataacactaagtaacccactaaattacacacacttaggaattacggtcacactccctgatggaactctgtttgaagcaagagcagaagttggaattcttacaagaaatattttaataagaggatctgataatgttgagtggaataacaaaattcctgcatgtcctgatggatttgacacag

SEQ ID NO: 651 - DNA, 137 bp, homo sapiens
gagaatttgctacacagacctgtctccaaggaaagtttggagaagaaataggaagtgaccaatttggaggctgcgttatgtttcatgctcctgtacctggtgctaacatggtaactgggagaatagaatatgtagag

SEQ ID NO: 652 - DNA, 1030 bp, homo sapiens
gtattccatgctggccaggctttccggttggggcgatatccaatacattggcacctgcttggagacttacagtttaaatcttatgtaagaggctgtgcaattcaccaggcctataacagagctgttactattcataacacacaccatcttctggttgagaggaatattatatatgatattaagggaggagcattttttatagaagatggtattgaacatggcaatatcctccagtataacttggcagtatttgtacagcaaagtaccagtcttctgaatgatgatgtgaccccggctgcattttgggtcaccaacccgaacaataccatacgacacaatgctgttgctggtggcactcactttggcttttggtaccggatgaacaaccaccctgatgggccatcctatgacagaaacatttgtcaaaaaagagttccccttggcgaattttttaacaatactgtccattctcaaggttggtttggaatgtggatctttgaggaatatttccccatgcaaacgggatcttgtacatctacagtgcctgcacctgcaatatttaactcacttactacttggaattgtcaaaaaggagctgaatgggtcaatggaggtgcccttcagttccataactttgtgatggtgaataactatgaggctggaattgagactaagaggatcctggctccttatgttggagggtggggtgaaaccaatggagcggtgattaaaaatgccaaaatagtcggccatcttgatgaactgggaatggggtctgcattttgcacagcaaaaggcctggttctcccatttagtgaaggcttgactgtctcttctgtgcactttatgaactttgaccgtcccaactgtgtagctttgggagtgacatccatctctggagtttgtaatgacagatgtgggggttggagtgcaaagtttgttgacgtccagtattctcacacaccgaacaaggctggctttcgctgggaacatgaaatggtaatgattgatgttgatggctcacttacag

SEQ ID NO: 653 - DNA, 192 bp, homo sapiens
ggcacaaaggacataccgtcattccacacagctcattgctagacccttctcattgtactcaggaagctgagtggagcattgggttccctggatcagtctgtgatgcttcagtcagctttcaccgtttagcgttcaaccagccttctccagtatctctgcttgaaaaggatgtggttctttcagactcttttg

SEQ ID NO: 654 - DNA, 152 bp, homo sapiens
gcacaagcattattccatttcagaagaaacgactgactcatatgtctggatggatggctctgattccaaatgcaaatcacattaactggtattttaaaggtgtggatcacataaccaacatttcatatacatcgacattctatggattcaag

SEQ ID NO: 655 - DNA, 160 bp, homo sapiens
gaagaagactatgtaattatatcacataacttcactcaaaatcctgacatgtttaatattattgatatgaggaatggttcctcaaatccattgaattggaatactagcaagaatggggactggcaccttgaagcaaacactagtactctatattacttgg

SEQ ID NO: 656 - DNA, 172 bp, homo sapiens
tgtcaggaagaaatgaccttcatcagagtcagctcatttctgggaacctggatcctgatgtgaaagacgttgttattaatttccaagcttactgttgtattctccaggattgctttcctgtacatccgccatcaagaaaaccaattcccaagaagcgaccagccacatataa

SEQ ID NO: 657 - DNA, 130 bp, homo sapiens
ggaggtagattaatcggtggctgggaagataacccttttaaaggagacttaaagattgttcttagaggaaatcatactacacaagactgggctcttccagaaggaccaaatcaaggggcaaaggtcttag

SEQ ID NO: 658 - DNA, 119 bp, homo sapiens
gggtgtttggtgagctggatcttcatggaattccacattcaatatataaaactaagctctcagaaactgcatttgcaggttccaaagtcctgtctctgatggatgctgtggattggcag

SEQ ID NO: 659 - DNA, 130 bp, homo sapiens
gagggagaagagattgtgataacaaccacaagctacgatttccaccagacagaaacaagaagtatcgttaaaatcctgcatgatcataaaattctcattcttaatgatagcctttcctatactcactttg

SEQ ID NO: 660 - DNA, 174 bp, homo sapiens
ctgaaaaataccatgtccctggaactggtgagagctacacgttagcagctgatgttgggatactgagtaggaacatcaaaatagttggtgaagattaccccggttggtctgaggactcttttggagcacgcgtactggttggctcattcactgaaaatatgatgacatttaaag

SEQ ID NO: 661 - DNA, 104 bp, homo sapiens
gaaatgcaagaataagtaatgtggaattttatcacagtggtcaagaaggcttcagggatagcacagatccaagatatgctgtaacgtttcttaacctaggacag

SEQ ID NO: 662 - DNA, 112 bp, homo sapiens
agctcattaattgttggaagtagccctgggtttaattgctctgatgtcctaactaatgatgatcctaatattgaactcactgctgctcatcggagtcctagatctccatcag

SEQ ID NO: 663 - DNA, 117 bp, homo sapiens
gtgggagaagtgggatttgttggcctacctttgcttcagctcataacatggcaccccgaaagccccatgcaggaatcatgagttacaatgccatcagtggccttttggacatctcag

SEQ ID NO: 664 - DNA, 168 bp, homo sapiens
gaattattagagattcaacctgtaagtaccttccagagtggcagagctatcagtgctttgggatggaatatgcaatgatggttattgaaagtctggatcctgacacagaaactcgaagactttccccagtggctataatgggcaacggttatgttgatcttattaatg

SEQ ID NO: 665 - DNA, 147 bp, homo sapiens
gtgactgcccagccagttgaaaggtctgcatttcctgttcatcacgtggccttcgtgtcctcactcttagtgatcactcagccggtggcagcacagccaggacagccatttcctcagcagccttcggtaaaggcaacagattctgac

SEQ ID NO: 666 - DNA, 154 bp, homo sapiens
ggtaactgtgtatcagttggaattactgcactaactttgagggccatactcaaggactccaataataaccaagtcaatggccttagtggaaatacaacaattccgtttagcagctgttgggccaactacacagaccttactccccttagaacag

SEQ ID NO: 667 - DNA, 1192 bp, homo sapiens
gaagctactaaagtgctgttccgaagaataggctgaaacaaaaatataagaattattagctactttgttgggcaataggcaaaagtctatagcattttcatgaaaatatactaaaaatatttttatgatatataaaatgtactaattagctttaaacactaaaatcagatttcttcaaaatataaatttgttttgattctttatatttatatgtttttatttcatttcaataaacttccagaaatttgtcatttggaagtagatatgacacctctaagttattgtaccaacaaatcatagaatcctttaaataatggaaagagcttgtctagttccgacttaagctctttacatcagattggaagcattttaagtattctttttaatacttgagggggggggttcttattttctctatccatttacaaggttttgatttccaataatctaagccattctagtttctaaggattttggagagacgagattgtcttcacacaatctgagatgacaatctcatctcagatttttcacactgagttcctgtctttcagattcacagtaatcccaggaattctagactgtcctgtatttctggatctgcccaactctggccaatattgggaaaagttttcttgtccttggaatgaacttttggaacaagtccaaactccttctctgcctacccctccttcccactcaaaactgccactactaagagcaggactcccctcaaccccgggcatgagttgagggttgaggggttcccttaactggtggatgcttattcactgtttactggatgaatgaagtataaagtgtgggaagtcaacaacagaacagaacttattttcaagaagataaattagagaatgcaaaaaagctacagactaaggtagctaagttaaccgaactctctaacagtattgaaaatagcaattctttactcagaaaattctaaagggaatacttaatttgacagaactcctaataaagacattgtagccagatccagagccttccagcaagtgacactcagcaaacatttgggacagctgagagaattcaacaaagcccataccgttgtctcttgtataacatctcctagtacattcacctcagatcattgttagtcttacttagcagaaaatgtataaacacttaccaggtagtaaaatcaaatatcttggta

SEQ ID NO: 668 - DNA, 34 bp, homo sapiens
cggcggcccgcagcggggctccttgcacagcagc

SEQ ID NO: 669 - DNA, 144 bp, homo sapiens
aggtaaattttgagccatttgttgaagatcactgtggaacatttagttcatttatgccaagtgcctggaggagtcgggattgtgagtccaccttgccatatatatgtaaaaaatatctaaaccacattgatcatgaaatagttg

SEQ ID NO: 670 - DNA, 195 bp, homo sapiens
aaaaagatgcgtggaaatattatgctacccactgtgagcctggctggaatccctacaatcgtaattgctacaaacttcagaaagaagaaaagacctggcatgaggctctgcgttcttgtcaggctgataacagtgcattaatagacataacctcattagcagaggtggagtttcttgtaaccctccttggagatg

SEQ ID NO: 671 - DNA, 33 bp, homo sapiens
atattcacactgcagaggcgctgccagaaaaag

SEQ ID NO: 672 - DNA, 752 bp, homo sapiens
aaaactctgaaatcaatgcgaataatagtttttgcattaatgtatctctactaaaatttgggggaattttaaaactaatctggtatctattcagacatttacctgcactcgtaccattaagaaagacagaaagaagcaaaaaaaaattaatcttgtatatgagaggaaaaggaaagggcttctgagaggattcttagttgtttcttttgaattcacttttaatagcaggatttggaaaatactaatttctgtgcttaagggtcacaggttctgggctctcaactgatatttaaggtgacattcatttttattaggtctaacatctcaagctaaaggagaaagaaaaataccttcttttaaatggcaaagaccttcattagcagcacacttttataaacaaccatatagttaaaatgtggccttaaactttcaattactaaatgatgattaagttggatattttaaaatgtcttatacataagcttaaagtaatatattgaaactttaacagttgtactgttaaaactcatggactttctgaaaaactaaatatataaaataatatgtaacactcttaataactggaagatataaaatgtaacttggatttaaaggagaagagctaaagatctgaattatagtctcattagaaccagacagatgttgttgagaagtactaaataaattagtaatctagatatctttattatgtaaatgagtttaggtgttctatttaataaactattttctgg

SEQ ID NO: 673 - DNA, 259 bp, homo sapiens
tccggtcctgccacaagacgagggggcatggcctctgcgagtcactctgctacaatccagcttctaatccgcccaaaagcggcagccaatcggagcgcgaggacgtggtctggaggtaccgccggagatctgggaccactcagggcatcagggggcgtggtctggtccccattgcgggcccgggaggggaatggtttctatggccaaagtttggttttctcaacactgtctaaatttggattaaaactttgaactttta

SEQ ID NO: 674 - DNA, 455 bp, homo sapiens
gtggaaatttggactcccagggagaccctcttcccggtgtgccgctgcctccttcggaccccacgcgccaggagacccctccccccagatctcccccggtggctaattcgggttccacggggttctctcgccgagggagtgggcgtggaggaggtcccaccccctgggggcccgcgtgggatgccgggatcgcccctccggtcctgccacaagacgagggggcatggcctctgcgagtcactctgctacaatccagcttctaatccgcccaaaagcggcagccaatcggagcgcgaggacgtggtctggaggtaccgccggagatctgggaccactcagggcatcagggggcgtggtctggtccccattgcgggcccgggaggggaatggtttctatggccaaagtttggttttctcaacactgtctaaatttggattaaaactttgaactttta

SEQ ID NO: 675 - DNA, 94 bp, homo sapiens
tggccacacacccctgaccatcacaggcttcaacctggatgtcattcaggagccaaggatccgagtcaaattcaatggcaaagaatctgtcaat

SEQ ID NO: 676 - DNA, 94 bp, homo sapiens
tggaagcactgccatcactgtgagtgggacccacctgctgacggtccaggagccccgggtccgtgccaagtaccgcggcattgagaccaccaat

SEQ ID NO: 677 - DNA, 170 bp, homo sapiens
tgatcctgtacgactgcgccatgggccacccggactgcagccactgccaagcggccaacaggagcctgggctgcctgtggtgtgctgacggccagcctgcctgtcgctatgggcccttgtgcccgccgggggctgtggagctgctgtgtcctgcgcccagcattgatgca

SEQ ID NO: 678 - DNA, 61 bp, homo sapiens
catccatcccacctgccactaccttgggtgtccttacagctctgccctgaccccagcccct

SEQ ID NO: 679 - DNA, 90 bp, homo sapiens
aagtacatgggtgtgttcacgtacgtgctcgtgctgggtgttgcagctgtccatgcctggcacctgcttggagaccagactttgtccaat

SEQ ID NO: 680 - DNA, 94 bp, homo sapiens
catcaagtacatgggtgtgttcacgtacgtgctcgtgctgggtgttgcagctgtccatgcctggcacctgcttggagaccagactttgtccaat

SEQ ID NO: 681 - DNA, 34 bp, homo sapiens
cgggccaggttgtccaggtcactctgactgctga

SEQ ID NO: 682 - DNA, 136 bp, homo sapiens
taacgtcatcctgttcccctgggaacaacagcaggccttcatctcctggctcaggactccacgccagcagccccactgcatctcaaaataactccaaagcagcagtgaactccgcctccagttttaactcttcagg

SEQ ID NO: 683 - DNA, 85 bp, homo sapiens
atcttggtaccgatggaatcattccacctacctccactgagaccaaaaagtttctcctactccagctggccctgtattcccctgg

SEQ ID NO: 684 - DNA, 34 bp, homo sapiens
ggaagaatcaaatcaaggatctactccaagtgac

SEQ ID NO: 685 - DNA, 34 bp, homo sapiens
tgcacagagccgcagggctgagaccacgtccatg

SEQ ID NO: 686 - DNA, 123 bp, homo sapiens
ggctacaataaggcagtggattggtgggcattaggagtgctaatctatgaaatggcagctggctatcccccattctttgcagaccaaccaattcagatttatgaaaagattgtttctggaaag

SEQ ID NO: 687 - DNA, 1111 bp, homo sapiens
cacaggccgctgcctcagcatccaccccgcagcccacgtgtggcaagccggggaaggggtggagtgaacggccggagaccacgtggagaaaggggccgctttggcccttccatctgggtgccgggagcccctaggccctccggccatggccgacagcggcgatgctggcagctccggcccctggtggaaatcgctcaccaacagcagaaagaaaagcaaggaagccgcagtgggggtgccgcctcccgcccagcccgctcccggggagcccacgccacctgcgccgcccagcccggactggaccagcagctcccgggagaaccagcaccccaatctcctcgggggcgccggcgagccccccaaaccagacaagttatacggggacaaatccggcagcagccgccgcaatttgaagatctcgcgctccggccgctttaaggagaagaggaaagtgcgcgccacgctgctcccggaggcgggcaggtccccggaggaggcaggctttcctggtgacccccacgaggacaagcagtagccccaatagcctgcgcgctccaggactgcctacccagcactaccccaaacccccagttccaaacccgagacttcaggcccgcccccttacgcgttgtctcattccaccaaattcagaatatttacacaatgccttcatgattttatttttctggaaattgaagtgtcaattgggttctcaatatttcatgactccaaggatgcattaaatatttatttgtggtaagagaagatacctgccgcggaggagggtggcataattatttttttttcaaaaagctattctggggccctggccccctggctcctgtcacagctcagcttgaactctgtagcctctcaaatgaagaaggtggctgttatttaggactctgtggaaagcaaatcacagtgctgtttttaatgcctgagaaatgcactttaggggtagacttataccttaagtgaaggagtgggggaggaagaagcattcttaagtagaagcattttaaaataaggcaggattgcttttaatgttaaactacaaaactgtacagcctattttagtgtggactattaaaacccttgcactgtaagg

SEQ ID NO: 688 - DNA, 35 bp, homo sapiens
cttcatgtggaccccagtgtcataagggaactcca

SEQ ID NO: 689 - DNA, 277 bp, homo sapiens
gatcagcgaacaagaggagcacgttaagggggaagatgaagacatccttgggcctggatatacggaggactccaccgacgtgtacagctcccagtttgaaaccattttggacaacacttctttatactacagtgcagagtccctggagacattatactcagagcctgatagctattttagctttgaaatgcccctcactccaatgatacaacagcgcattaaagaaggtggtcagttcttggagaggacatcagggggaggacatcaggatatcctg

SEQ ID NO: 690 - DNA, 119 bp, homo sapiens
agtgtgtctgcagatggtggcatcgtgatgggctattctagtggcgtcaccaatgggctgaatgatgccagcgactccatctacacgaaaggcaccccggagattgctttctggggaag

SEQ ID NO: 691 - DNA, 34 bp, homo sapiens
gagagagacagggaaaggatcagcgaacaagagg

SEQ ID NO: 692 - DNA, 195 bp, homo sapiens
caatgctggggtgaaaacaacacggctagaagctcattctgaaatggggagcactgaaattttggaaaaggagaccccagaaaatctcagtaatggtaccagcagcaatgtggaagcagccaaaaggttggccaaacgcctttatcagctggacagattcaaaagatcagatgttgcaaaacaccttggcaagaa

SEQ ID NO: 693 - DNA, 59 bp, homo sapiens
atggagtccattgccttacctgtgcaataatgcttcttaataccgatctacatggccac

SEQ ID NO: 694 - DNA, 194 bp, homo sapiens
agatgatgaagagaaaaaaaagtctccctcagaaagtactgaggagaaagctaacggaacacatccaaagaccatcagtcgtattggaagtactactaacccatttttggacattcctcatgatccaaatgctgctgtgtacaaaagtggattcttggctcggaaaattcatgcagatatggatggaaagaaga

SEQ ID NO: 695 - DNA, 144 bp, homo sapiens
gaggagcaactgaagtcacatgaaagtaagctgaagcagatcaccaccgagctggccgagcaccgctcatatccccccgacaagaaggtcaaagccaaggacgtcgatgagtacaaactgaaagaccactatctggagtttgag

SEQ ID NO: 696 - DNA, 251 bp, homo sapiens
aaaacccgctatgaaatgtatgtcagcattctcaaggaaggaggcaaagagctactgagtaacgatgaaagcgaggctgcaggactgaagaagtcgcactcgagtccttcgctgaacccggatacttctccaatcactgccaaagtcaagcgtaacgtgtcagagaggaaggatcaccgacctgaaacaccaagcattaagcaaaaagttacttagagtccatctgcggccaggaagtgctggtcatggag

SEQ ID NO: 697 - DNA, 6441 bp, homo sapiens
caaaatagggtttttcaagatctttctggtaatccgtgaatatatttaaaaaaaaaaagtctgtgacaaaacggtgcattagtaattttttctattgtatatttttgttagtttctgtacagattgtctttgctcttgatttcttttgctttgatgatttttgcaacttgatagctaatgcaccttttctgtgaggaggaggggatcgtgatttcagaatgaattatgtatcccttctcttttggttttctcttgtttgcagtctgctcagttgttttatgtattctcatatcaactgttaaacttttttttaaggttaaagaatttaatccattgtgaaacacttaactggacaaactgtagttttagtaaattctagctggagttaatatacgcctttatatgtgaaatcttgcccagtcacagaggtagaattgagcactcacagatgctccagtaagaatcacagtgctgggaatctagttgctccaatatgaggcagcttcatgtgcagcttagcacttgttgttgagatcggaccctgctggaagcagggaaaagaagcgtgaagatcgtaggattgagaacttagggaagcacattagcttgcttgaagtgctgattccatttcagccaagcaagggaaagaggaagtggagtcattttgcctttgaaggctgaggaaagattgatacccagttaattttgtttgctaaaggatgggggcaataatcggcccttgaggagctgcagcagtaggcatgtgctcagtctgcaggaattgttacctcactcccacagggtctagactagaaatccatcatctctatcgttgatatccttccatccaggaatagatttttcttactctacatatgtgtgtgtgcgtgcgtgtgtgtgcgtgtgtgggcatggggttgtgtcctggttgtgatattgaggtcttccttcctaacaaattaatactaaaatgaaacagcttttcttgtgtccttaagacaaaataaggaaggaaaacgtagctgcagttgtccacgatggatattggttctttaaaatatatctgaaagtagtagtcagaatgaattatggttggaaaactgaggaatcttctggttgcaggtgcaaagtgactttgtttattcttgtctcagtctccttgatagccacttcactctgctactactcaactttctcctaaaaatacttcatctattttcagtcctttctttctgtctactcaaaatggttctattaactttgcagtcatgagcttgttccagttacagtccctttgaagttcagggtgataaacagaatattcttctgtagaggaagagaaaggagtgaaagtttagcccactgagacctagagctttgtgatttcctaaccttgaaactctgtaatccctaaagttaaaatctccgcaagtggcacaacttcagaactaatagtatcactttgatttttctttttcctcccttagaaagtttctctagttctatagtttatttgttgaaggtactatgaccaaagaatcagctgctctacaggaatagcatggttccagtgaattagagaaaacctgctgtaaagccatggtagtgtctaagtggtatgttattatgatgtactagcatttatttacagaattatttattaacgtttacttccttcccctctgtaaatgtccatgactattgcccagagaaggcttacccctctctagggttgcagttgctttctttgtaataagtattttgccacacctgtaaaaaaaaaaacctcacttttaactctctgccttgtttgggtaaaggcagtaactaagtttatgtttcagaactgcaaaacaaacaggatagttaccaatatggcccatgtatcagattgatttttgtagcctctcactgaatccaacatatccacaagcaagttatctgtctttctacctgataatctaaattatcaggatatttgttttctgcctaaatgtttatactaagccgaggggagagaggtacctagaccatgtcatctacaagcttcagtaactaaagaaaaaggaacttccctgagtggcttgaatgtgtttgcccacagtctatatctatgtatatagaatgtctgtatgtattttacttatttaatatacattgaatggtaccttgctacagtatttctgacatttagagtagtgttgaaatactcggctagcatcagcaccactatagcactgtccgtgtcatatgagtcactaatattaactccagggacttctggataggctaatagatcattggatacgaagggctcttttgaagcttcagtataccatgtttgcatagtttatctttaaaaacaactttaaaggttcttttgtgagccaggatctcagactgccgtagcatgatgctgtccatctttagcgcatgggctgagaacacctcttccctgaggcttctgaaggttgctgtctgtcatgagtgcatgaaggaggccaagagtttatgctatgggaggaaacagtcactgatttgcctagattctgagagtctggcccatagccaaccacattttcctttgggataatttatttcctgtggcatctagccagaagaaattgaggatgtttcctttcacagctgctccaagcctgttgcccaattcacggtacaagggagcaccccttccctttcctctgaaggtacgccacccacctccgtcgcccacctcagcgcccaggagccttgggacttccttccatatgataaatcattcttcttcacgtcaatacacttcatattaatttctagtacagaaaatcttgacagctatcagaatgccttggtcatagtgttgttgcaaaattgaccatacaggtggcccatgtataaaatctgaattttaggggtttgtccccacctcgcatgctggcttttacagggaggtgtctgggattcctcattagcaatcaaaacttaattactgggatgcagagtccttactttatcgccagcccgtaggcatttctgaagtgcacttttttgaaacatcattttgctaactctcagcagtgtctaattaaactgagcaatacttttgtgaattttaattaatctcagcaaaaccatgatgggagagagtcctctgatggaaatgtagtccctggattatgtgtaacctttttattcctcttagatgcagaggatagaaagcattttttggtgcagtggtcttgtggcaaacacaagaccctctatgcgtctccaactgttatcctaatctagaaaatgaggactggcccctgggcaaaagtgacatgaggaatttactctggaagaggaaaatctgggtggctttccaaggctaagataggtttgtatttcaccctgtggccaagctacagaacttctgagattgtggaagaatttttgcaaccagcagggaaagaggcctcttactgcctaaacacaaagttacactgagcttttctactgtcctttgcctattgctccctctatcatgtaaagatctgggaaggatgagaggcagggcctgcttgtcatgagctgcactcttttctttttaactaatcattgacaattggaagaaaattgacgttaaagaagtttctccattgtcttactaacaaaaccttttgggtttcattaattgtccttgaaattgagttcctttggcatttttccttgcagtcatcagttaagcatgttgcatcctgaattcacagaagtttagctttgcaggtttgaatctctgtaatttaactcccgtggacttggtcgagttttcagcaggttgggagccacctctcttcatttcagcagtgagtcatcccttgacttttcaaatgacagaattttttccaattgtaaaattagcactgtaaaacaaagaaccaaagtggcatcctaagagttgttaaacctgaagtctagtttatgaggaattgtccaagttggagtttaaatagtatctgcttttgtctcaaagcatctaagttattctgacagaaaatggtaagtcagctttgcaggcagatgcgcctctgggcctcctaccttgctccacagctttctggccatcttgtctcccaggccatgccactgctctgccacatgtcagcaaatttctttccaccagtcttatagcatcttacatgatcaaatcatcacagaataaccccgtgatagattattgatagcaatagagaggggctttgtcactgatttttctctcagattccttttccatctctcatccataaaggaaggactgaaatccaaaggcattctccttttgtacctacagtatccagaacccacgtgggcagccttctgcttatgacaataattggcccattgcatgcagagagaatgtcttcatagagagaatgtcattaaatacttgaatctgcatgacagtttgacttgaatgcaacagcaggaaaattttgcaagttacataattgtatatacagtaggttttcttaagtctcttcggttcatcctttgtaatttgtgtgtgtatctgtagtattgcaggcttttggagactattcttacaggcagtatgtcagtcatcaaagaaaatgctgtcacctgccattgttgtatttgtgggtatttatagttgtatgtatgtaaatgcatcagtgtgtagattgcatatcagtgtatggtacatgtacatcaaaattatttttgtccttaatcagtgtgatatgaaaagcaagtacaacctcataggactgattatataatgaagttgttgagagtatatatagtggtattgttttattaaacttaaactcaaataatattttgattaaaatttttaataagactttatgctagaaaattctttgagctttgaatcaccagggcaaaaatgactttcaactaaccttgtgaatcttttgcagtgtactgtgtgcaataccaagggcatagctccctgtaatttgggaaatacagaaagaaaagaaaaaaaaaaaaaaaggcagcctgtgcagtcttagtaactttagtattaagagcacttaaagtcaaactgacaattttgggcttattacaaaatgtgatgctttaaagcacacgttctttattgttgttgtaattagtccataaaaaatatagctttcggaagaattaagtacccaccatatcatttatgtatttgtgtatgttttacgggagatcaaaccactctcgtggtgccgcatccgtactcgcttgacttggaagaaatatcacaagcactaaagtatatcagggcatcccaggattgggtactgtatcctaggtttgcagttgcagaaattagcatctagtgtcacaggtaaaagaatttcaggaccaggtttaaactttattttaaatatttttatacttaggtctctttttcctgcctctccccaaagaagagccactggccttagttgtttgagcttactgcttatattatagagtgtaaataggtaactagagactaaaattttattaaccagcatgtttggtatatttaaagcagtgactgagtgtgtttgagtgagtggctgagtgcagtgtcttttgtttaaacacactgcctcgtgtctttgtagctgattcagagagtttgaattgtggggtgggagactaacttcagctccaggctgcagtaatgtgttggtagttacacttgaggcatttttttgttgttgttaattaactctatagtctcaaactatttttgcaaatatatcatttttcctaattggttcttgacgtgcagtggactggctctgtgaatgattggcagggtcttagttttgcgagagtatttccttctaagaattattgtgatctgcagaaacagccatttgattcaaaaatcatgtagaaaaggagtaggagaagcaaaacgtttcatttttgggccttaaccatttgaaatgtttggactttaaacataaagccatggagtttataaagccaagtaaccatttgatatggataataatatctactctagagagagtatatatatgcacattgatttttaatgctgttaagatacttttgtaaaactgtaggaacaagagtaattagaccaaattgaagcttaggggacagtaaagtggttgctttccatttagggtaaccatgcatgtggttagtcctctcctcctgagattcagaaccagttgactgtccccttaggtgtataaggagaaaagttgacatgtctgggacctctgacatgtgtacacatgcttgcacacatgcacacacagtgaatgttttaagttatacaaacataagaccttaagatgcaaagagccagaatattctaaagaggtgatgaacagagggggtggaaactgcatcacagatgttttccaagggccagggtggaatctgagctctagtgtctga

SEQ ID NO: 698 - DNA, 149 bp, homo sapiens
gcctttccgttccgaacgtccacggcgccctggcccccctggccatcccctcggcggcggcggcagctgcggcggcaggtcggatcgccatcccgggcctggcgggggcaggaaattctgtattgctggtcagcaacctcaacccagag

SEQ ID NO: 699 - DNA, 34 bp, homo sapiens
ttgccatcaataccaaagttggccaacagcgaaa

SEQ ID NO: 700 - DNA, 77 bp, homo sapiens
atgaccactctagagtccacctgacaccggttgaaggggttccagattctgattacatcaatgcttcattcatcaac

SEQ ID NO: 701 - DNA, 34 bp, homo sapiens
acagcgacaaagacttgaaagccaccattagcca

SEQ ID NO: 702 - DNA, 177 bp, homo sapiens
actgtttcagactccaagccagatttgtcactccagctgatcttctttgatggtgaagaggcttttcttcactggtctcctcaagattctctctatgggtctcgacacttagctgcaaagatggcatcgaccccgcacccacctggagcgagaggcaccagccaactgcatggcatg

SEQ ID NO: 703 - DNA, 32 bp, homo sapiens
gaggaagccatagcactcgcagagaaatatgg

SEQ ID NO: 704 - DNA, 679 bp, homo sapiens
aattaatttgttgcagctttgtaaatatttctttaagattcagcctgagagttaggagaaatatttcagagccaaaagtgccttatacaaccttagcctattatagtaaatcattcaaggattcagaattttgcagtcacagaagagtgtatttattatgtagaatgaatgagggtactgtcacctgccttaatgtaggtaggcccagagtcttacatttaagatcttacatgcagttataaaaccgccacagtcttcaatccagatttgaagactcatgccataggtgacattctaaaataccattaaagccacttaaatgttaaataagaatatacatgcacatcagctcaatgtctttgagtattaattttatgtaagcattctatttaacatgaatataggacaaatcatggctatatctatagaccttggataaactggattgaccaattatacactcacggtgacttttttattggtgggaaggggattggggtggggcaggctggcttaatgtaatatgagcaaccaaagtgggacttctgtctccccgctatattcccattgctctgaatggttgattgaagggtcagggaactagattttatggctttagttcactgtgattgtacatttatacttggcctatgtgctggccgcacctgaacatagctgg

SEQ ID NO: 705 - DNA, 147 bp, homo sapiens
ctgcaaaccagtgtgccgtgccagccaaggacagggtggactgcggctacccccatgtcacccccaaggagtgcaacaaccggggctgctgctttgactccaggatccctggagtgccttggtgtttcaagcccctgcaggaagcag

SEQ ID NO: 706 - DNA, 136 bp, homo sapiens
ggaatccctggagatagagaaagtcaggctgtggttgacccccctcaagcagctagtactggggctcctcagtcttcagcagtggctgcagctgcagcaactacgacagcaacaactacaacaacaagttctggag

SEQ ID NO: 707 - DNA, 128 bp, homo sapiens
tgacgggtcagtcttacgagaatatggtaactgagatcatgtcaatgggctatgaacgagagcaagtaattgcagccctgagagccagtttcaacaaccctgacagagcagtggagtatcttttaatg

SEQ ID NO: 708 - DNA, 1056 bp, homo sapiens
gtgagacaaatggctgccgcactgctacgacggcttttgtcctctgggtttgaggaggtttatccaaatctgcctgctgatgttcagagagatgtcaagattgaactgattctggctgttaagttagaaacacatgctagcatgaggaaaaaactttgtgatatttttgcagtgctggccaggaatttgatagatgaggatggcactaaccactggccggaaggtctgaagtttcttattgattcaatctactccaaaaatgtggttctatgggaagttgcacttcacgttttctggcactttcctgggatttttgggacccaagagcggcatgatttggatatcatcaaacggttgttggaccagtgtattcaagatcaagaacatccagcaatcaggacattatccgctagagctgcagctgcatttgtacttgctaatgagaataatattgctcttttcaaagactttgcagacttgcttcctggaatcttacaggctgtgaatgactcatgctaccaggatgatgattcagtgctagaatcccttgttgagattgcagataccgtacctaagtacttgggtccttatttagaagatactctacagttgagtttgaagttatgtggagactctaggcttagtaatctgcagcgccagctggccctcgaagttatagtgaccttgtctgaaactgccactccgatgttgaaaaaacatacaaatattattgcacaggcagttcctcatatattagcaatgatggttgatctacaagatgatgaggactgggtaaatgctgatgaaatggaagaagatgattttgacagcaatgcagttgctgcggagagtgcactagacagactggcttgtgggcttggtggaaaagttgttttaccaatgaccaaggagcatatcatgcagatgcttcagagccctgactggaagtatcgacatgctggattaatggccttatctgccattggagaaggatgccatcaacaaatggaatcaattctagatgaaacagttaactccgttttgctttttcttcag

SEQ ID NO: 709 - DNA, 113 bp, homo sapiens
gattccagaggagaaccagccacccagcaagcaggtgaagccactcttccgccacttccgccggatagactcctgtctgcagacccgggtggccttccggggctctgatgaga

SEQ ID NO: 710 - DNA, 71 bp, homo sapiens
ccagtagaagatagaaggcgtgtacgagctattctaccttacacaaaagtaccagacactgatgaaataag

SEQ ID NO: 711 - DNA, 147 bp, homo sapiens
agaagagcaagttagtaccttgtaccaaaacatcaaccttgtggagccaagattaattcagccatatgaacatgttataaagaacttcatccgtgagatcagacttcaaagcacagaaatggaaaatttggccattgcggtgaagag

SEQ ID NO: 712 - DNA, 116 bp, homo sapiens
gaaaaccagaaagttaagaaagaccttttagaagcacagacaaacatagcctttcttcagagtgagttagatgctttgaaaagtgattatgctgatcagagtctgaatactgaaag

SEQ ID NO: 713 - DNA, 69 bp, homo sapiens
ggatctggaaataatccgagcatacacagaagatcgaaatagtcttgagaggcaaattgaaatactcca

SEQ ID NO: 714 - DNA, 85 bp, homo sapiens
aacagctaaccggaagctacatgacagtaatgatggccttagaagtgcccttgaaaacagttatagcaagttcaacagatctttg

SEQ ID NO: 715 - DNA, 173 bp, homo sapiens
gaacatatgtgatgaccgtaacagcaattgatgctgacgatcccaatgccctcaatgggatgttgaggtacagaatcgtgtctcaggctccaagcaccccttcacccaacatgtttacaatcaacaatgagactggtgacatcatcacagtggcagctggacttgatcgagaa

SEQ ID NO: 716 - DNA, 138 bp, homo sapiens
aaagtgcaacagtatacgttaataattcaagctacagacatggaaggcaatcccacatatggcctttcaaacacagccacggccgtcatcacagtgacagatgtcaatgacaatcctccagagtttactgccatgacg

SEQ ID NO: 717 - DNA, 186 bp, homo sapiens
ttttatggtgaagttcctgagaacagggtagacatcatagtagctaatctaactgtgaccgataaggatcaaccccatacaccagcctggaacgcagtgtacagaatcagtggcggagatcctactggacggttcgccatccagaccgacccaaacagcaacgacgggttagtcaccgtggtcaaa

SEQ ID NO: 718 - DNA, 254 bp, homo sapiens
ccaatcgactttgaaacaaataggatgtttgtccttactgttgctgcagaaaatcaagtgccattagccaagggaattcagcacccccctcagtcaactgcaaccgtgtctgttacagttattgacgtaaatgaaaacccttattttgcccccaatcctaagatcattcgccaagaagaagggcttcatgccggtaccatgttgacaacattcactgctcaggacccagatcgatatatgcagcaaaatattag

SEQ ID NO: 719 - DNA, 143 bp, homo sapiens
atacactaaattatctgatcctgccaattggctaaaaatagatcctgtgaatggacaaataactacaattgctgttttggaccgagaatcaccaaatgtgaaaaacaatatatataatgctactttccttgcttctgacaatg

SEQ ID NO: 720 - DNA, 234 bp, homo sapiens
gaattcctcctatgagtggaacaggaacgctgcagatctatttacttgatattaatgacaatgcccctcaagtgttacctcaagaggcagagacttgcgaaactccagaccccaattcaattaatattacagcacttgattatgacattgatccaaatgctggaccatttgcttttgatcttcctttatctccagtgactattaagagaaattggaccatcactcggcttaatg

SEQ ID NO: 721 - DNA, 234 bp, homo sapiens
gtgattttgctcagcttaatttaaagataaaatttcttgaagctggtatctatgaagttcccatcataatcacagattcgggtaatcctcccaaatcaaatatttccatcctgcgtgtgaaggtttgccagtgtgactccaacggggactgcacagatgtggacaggattgtgggtgcggggcttggcaccggtgccatcattgccatcctgctctgcatcatcatcctgctta

SEQ ID NO: 722 - DNA, 103 bp, homo sapiens
tccttgtgctgatgtttgtggtatggatgaaacgccgggataaagaacgccaggccaaacaacttttaattgatccagaagatgatgtaagagataatatttt

SEQ ID NO: 723 - DNA, 1389 bp, homo sapiens
ggccttaaagcggctgacaatgaccccacagctccaccatatgactccctgttagtgtttgactatgaaggcagtggctccactgctgggtccttgagctcccttaattcctcaagtagtggtggtgagcaggactatgattacctgaacgactgggggccacggttcaagaaacttgctgacatgtatggtggaggtgatgactgaacttcagggtgaacttggtttttggacaagtacaaacaatttcaactgatattcccaaaaagcattcagaagctaggctttaactttgtagtctactagcacagtgcttgctggaggctttggcataggctgcaaaccaatttgggctcagagggaatatcagtgatccatactgtttggaaaaacactgagctcagttacacttgaattttacagtacagaagcactgggattttatgtgcctttttgtacctttttcagattggaattagttttctgtttaaggctttaatggtactgatttctgaaacgataagtaaaagacaaaatattttgtggtgggagcagtaagttaaaccatgatatgcttcaacacgcttttgttacattgcatttgcttttattaaaatacaaaattaaacaaacaaaaaaactcatggagcgattttattatcttgggggatgagaccatgagattggaaaatgtacattacttctagttttagactttagtttgtttttttttttttcactaaaatcttaaaacttactcagctggttgcaaataaagggagttttcatatcaccaatttgtagcaaaattgaattttttcataaactagaatgttagacacattttggtcttaatccatgtacacttttttatttctgtatttttccacttcactgtaaaaatagtatgtgtacataatgttttattggcatagtctatggagaagtgcagaaacttcagaacatgtgtatgtattatttggactatggattcaggttttttgcatgtttatatctttcgttatggataaagtatttacaaaacagtgacatttgattcaattgttgagctgtagttagaatactcaatttttaatttttttaatttttttattttttattttctttttggtttggggagggagaaaagttcttagcacaaatgttttacataatttgtaccaaaaaaaaaaaaaaaggaaaggaaagaaaggggtggcctgacactggtggcactactaagtgtgtgtttttttaaaaaaaaaatggaaaaaaaaaagcttttaaactggagagacttctgacaacagctttgcctctgtattgtgtaccagaatataaatgatacacctctgaccccagcgttctgaataaaatgctaattttgga

SEQ ID NO: 724 - DNA, 147 bp, homo sapiens
gagtcagcagaagttgaagaaatagtgttcccaagacaattcagtaagcacagtggccacctacaaaggcagaagagagactgggtcatccctccaatcaacttgccagaaaactccaggggaccttttcctcaagagcttgtcagg

SEQ ID NO: 725 - DNA, 140 bp, homo sapiens
tccttgtgctgatgtttgtggtatggatgaaacgccgggataaagaacgccaggccaaacaacttttaattgatccagaagatgatgtaagagataatattttaaaatatgatgaagaaggtggaggagaagaagaccag

SEQ ID NO: 726 - DNA, 845 bp, homo sapiens
gagagaaatatgaacttcacgcgggaacagagtcgacacccagcgtggtggttcatgtctgtgaaagtgaaactgaagaggaagaagagacaaaaaaccccaaacagaaaattgcccagacgaggcgccccgaccctccgaccgcagcgttgaatgagccccagacctttgattgcgcgctgtgaggcccttggttgtggtgcgaggcggctgccctggtgggctctggccatggcgctctgtgcctgcggccgatgcgttgctgcgaacagcataggtgagactctgccgagtgaggtataggtcttctcaccacgcctgtactgcagacacggtcgtgtagagtagcagctgatttgacctgtcccagattttaagtgatattccaaaagggactttacattaaaggagaagcccccaagatgtggccacccttaaccatttttaaatagtaacaaattaggaaaacagctcccctcccctccagccatgtaagtcctcctgattctgtatcacatgagacaccaaaaactggaaatgtagtcacacccagtacagtaccatttttatgaatttaaaaatagtcttataattttaattgtttgtgggtatttttctactggtataaatagcttctaattaataaatcgatcaaaggttgttactcagtatacatgaaattttttgcaggaaaagaaaccagaaatacagaataattcatagaaatcatggttctcaagtatttgttcaattagctttggggaagaaatgccaaatccactttgaggatgagtcttacgacacagatccacccacatgttgctcttatctggaatttgggacaa

SEQ ID NO: 727 - DNA, 110 bp, homo sapiens
caaagtttggtaaattttccggcaaagatgatggctctagttttaaagcagcgttgctgtcaggccctcctggtgttggcaaaaccaccacagcttccctggtgtgtcag

SEQ ID NO: 728 - DNA, 140 bp, homo sapiens
agcctccattatggcatgctgaatttaccaaagaagaattggttcagaagctccgttccaccacaaaaagtgcagatcacttaaacggcctgcttcgggaaatagaggcaaccaatgcagtccttatggagcaaattaag

SEQ ID NO: 729 - DNA, 3380 bp, homo sapiens
gatgaatttgacaagctgaggcctctctgctacaccaacacagacatcttcctgctctgcttcagtgtcgtgagcccctcatccttccagaacgtcagtgagaaatgggtgccggagattcgatgccactgtcccaaagcccccatcatcctagttggaacgcagtcggatctcagagaagatgtcaaagtcctcattgagttggacaaatgcaaagaaaagccagtgcctgaagaggcggctaagctgtgcgccgaggaaatcaaagccgcctcctacatcgagtgttcagccttgactcaaaaaaacctcaaagaggtctttgatgcagccatcgtcgctggcattcaatactcggacactcagcaacagccaaagaagtctaaaagcaggactccagataaaatgaaaaacctctccaagtcctggtggaagaagtactgctgtttcgtatgatgctggcaagacacccagaaaggctattttcagatgaaatcgatattagaagctatattagctgaaacaactccttttactgcgtagaacctatatcgagagtgtgtgtatatgtattataggaggagctctcaattttatgtattctttctgcctttaattttcttgtttgtttgagcttagggatgagatacttatgcaagatatttttgaagtaaattaaacatttttcacatctctggaaatttagagttctagacctctggttaatttatatctaatatgaagaagacacctctaatctggatgttaagaatgaagttctgctacattataatgtacagaagagcaaaagggaggaacactatggttaaccctctcttgattaagggctacttaatgcacagtgcattatgtacacaggtcaaccatggtaacaatagttcttagctttgaaactccatgcaaaccatgccttttttttaaggagcaaaaatctgagaaaaaaagtgagagacctctgcctacaaaacctcaaaccagtcacttttgtcaattgctaatacccagttacttatgatttaaaaacaaccaacagaaaacatcccactgactgtatggcactctgtagtcaaaaaaggaaacttccttattgggacttttctttcttagtccagttgtgttgacacatatgaacacagacaaagtgctatgcggaggaaagcaagtgttggtcagtagtttcatgttttagggagtggttcctgtggagatcagaaagtgacatttgctttcggtactgtaatacgtgcaccaaactgcctcaatcctaggtaacgagggcaacagggagcacctgtctggattgtttttaaacctccatactcaagctgtctcttcggcagggaggtgaatactcttgaaaggccaacagcaagtgtttgtgggacacaacacagataattttttcttaagtcggccaagatgtacttctctgtgtgcacacccatgcacactcatgcacacagatacataggtctgtatggctgtatttgctgttgattcagactttcacaccattaatggggaaaagcgtggccacaaaaacagatgctaggaagcttggcttcctcttcttgttgacccttttttgaaccaacatcttttttattatattcagagtatgtttttaagtgtatcttaatatatacattttttaggacatcttaaatctaaacaaaaaataaaatgaacatctcttgaaacctgttaaaacaaccagttaaagccacagatggctttcagggcagtagcagcagaggccagtggactctgaggactcctgaggggcggggcgtgtagccagccaggtgcatgccgggaccatggcccccatacttggctgcttcctgtgacagtgaaatacatccttcaaggtggcagctgttagggctgaatcttctggagaaaaaggtgccatctcaggagaatagcttttactctggtaggaatgcttccgagacaccacaaggcagcctgaacactcagttgcagggtcgggcttgcggtgggtgacccagagccaccaaagtcacatccacaactaatgagggaaatctgtaaagccagttagatagaagaattttatttttctgtgggttttgtgttgtcttttttatgttaaaaagaaatccagtttgtgtttttctatagaaaaagtaaaagatcaggttatactttaggttaggggttctatttattcctgttagtaaataaaattaacaaatttctttgtttaacaaaagattaatctttaaaccactaaaatacatagactgattgattattcaacacattggaattgatgtcggtcatagtttcctgaagcatttagttacaacctgaaggaataaaatgatttgtggaaatgcttaaaatagacctaactgaatacagtctcatcttgccgcgcctggcttacctatctgtggaaagctaggcttcccaggctgggctctgcctgtctggtgcctggaggtgtgggagggaagatgagttatttaactggtaagcgatttgaaacactatttttatattaaagtaaatggcatggagtatagtgcaaattcatttttaagatagaacacaaaacttgaaagaagttttatgcgtgtgacagtgtatggggctgcagttggtctccctggaggggacttccacacctcctgcctttaggccatgggtggaaagtgctcagtgaagtacacctgtgtggcccagttctgaaagctttatacagttgaattttaagtggggttgataacaccttggactgttagtgttaaaaatctagtgggttgacctttaaatgcaacagtttttaaaatatattgctgcattttatagaatagtaaaggtacgattatacttgagattttcctccatttttatttcttcgtgaacatagagtttggggccgaaaatgtttttaaagtatgtgtttgagttaaatataaagttggttcacttcaaagctaaaaaattgttaaacttgcagcttggtattgcagagaagattttataagaattttgctttagagaatgccactttggctgaactacaagtgtaggccaccattataatttataaatacagcatacttcaaaactgtttgttatctcttgttaccatgtatgtataaatggaccttttataaccttgttctctgcttgacagactcaagagaaactacccaggtattacacaagccaaaatgggagcaaggccttctctccagactatcgtaacctggtgccttaccaagttgtgcttttctgttttcaagtgtaaatgatgttgagcagaatgttgtacttgaaaatgctataagtgagatggtatgaaataaattctgacttatgaa

SEQ ID NO: 730 - DNA, 86 bp, homo sapiens
cccaccagtgaggaggacctctgccccatctgctatgcccaccccatctctgctgtgttccagccctgtggccacaagtcctgcaa

SEQ ID NO: 731 - DNA, 34 bp, homo sapiens
gccgcaacgtactccgggtcggccttgcgctcgg

SEQ ID NO: 732 - DNA, 112 bp, homo sapiens
tgctggaggtgagctctgccatccccatcatcatgggctccaacatcggcacctctgtcaccaacaccatcgtggccctgatgcaggcgggggacaggactgacttccggcg

SEQ ID NO: 733 - DNA, 237 bp, homo sapiens
ctccgatctagaggcagattcctgactaatcccagagggctggcccagcctgtgctccccgggctgctaggaagcgatgaccactcttgttagcccaagttgaagaaagccgggctgtgcctgggagccgagagaggcggtaatatttagaagctgcacaggagaggaacatgaactgacgagtaaacatgtatggaaattattctcacttcatgaagtttcccgcaggctatggag

SEQ ID NO: 734 - DNA, 248 bp, homo sapiens
gctcccctggccacactggctctacatccatgagcccatcagcagccttgtccacagggaagccaatggacagccaccccagctacacagataccccagtgagtgccccacggactctgagtgcagtggggacccccctcaatgccctgggctctccatatcgagtcatcacctctgccatgggcccaccctcaggagcacttgcagcgcctccaggaatcaacttggttgccccacccagctctcag

SEQ ID NO: 735 - DNA, 34 bp, homo sapiens
tcccgcaggctatggaggctcccctggccacact

SEQ ID NO: 736 - DNA, 118 bp, homo sapiens
gttaccaacatatgtcatgctgctgacaagcagcttttcaccctcgttgaatgggccaagcgtattccccacttctctgacctcaccttggaggaccaggtcattttgcttcgggcag

SEQ ID NO: 737 - DNA, 34 bp, homo sapiens
tgtactctttaacccagatgccaagggcctgtcc

SEQ ID NO: 738 - DNA, 130 bp, homo sapiens
acaaatgaccctgttaccaacatatgtcatgctgctgacaagcagcttttcaccctcgttgaatgggccaagcgtattccccacttctctgacctcaccttggaggaccaggtcattttgcttcgggcag

SEQ ID NO: 739 - DNA, 202 bp, homo sapiens
cctgacaaaggtcagctagccccttgaggacatcagctttggcctcagggtcctaatggcagcagaaccactgacagagctagaggagtccattgagaccgtggtcaccaccttcttcacctttgcaaggcaggagggccggaaggatagcctcagcgtcaacgagttcaaagagctggttacccagcagttgccccatctg

SEQ ID NO: 740 - DNA, 116 bp, homo sapiens
aatcgaccacggaaatttgacacctccgggcttggaagcagctctctcctccttccccgctgcttataaacctcagccctgaggctccagctcactctaccccatctccttgccgg

SEQ ID NO: 741 - DNA, 34 bp, homo sapiens
ccccatctccttgccgggtcagccctgacaaagg

SEQ ID NO: 742 - DNA, 208 bp, homo sapiens
cctgacaaaggtcagctagccccttgaggacatcagctttggcctcagggtcctaatggcagcagaaccactgacagagctagaggagtccattgagaccgtggtcaccaccttcttcacctttgcaaggcaggagggccggaaggatagcctcagcgtcaacgagttcaaagagctggttacccagcagttgccccatctgctcaag

SEQ ID NO: 743 - DNA, 31 bp, homo sapiens
ttattctgccccagctgatgtttgagccagc

SEQ ID NO: 744 - DNA, 108 bp, homo sapiens
cagatcaagtggactgcagcgacctttcagaacacaacggacttgacagggaagagtccatggaggtggaggccccggttgctaacaaaagcggcagcggcacttcca

SEQ ID NO: 745 - DNA, 34 bp, homo sapiens
tgacacagttaacaaaacagatcaagtggactgc

SEQ ID NO: 746 - DNA, 34 bp, homo sapiens
aattcctgccccatctgccagaagaagttcacca

SEQ ID NO: 747 - DNA, 8276 bp, homo sapiens
catcatctgagggttctccataaacacggttagccttccaagctgctgtcctgccagttgatatgaacatggcctgttcctcacagaaagactaacccatggcactgccagaagatatccttggatcgtggacagattaactggattaactgaaaaaagcacccattcagtcaatattatgggttttggaaaaatcttgctttgctgtggatttaaaatgaggtgcataatataatgccaactcattccaagtggtcattggcagaccattagctggaaaattgcttccagttttcatcactgtgcataactcaagttccctttggggagctgtctgtgcccttcactagaagtcctgttttagcatggcaactactggattattttattatgtgtgcagatggcatgatgttttagaaaaaaaaaattacagcaattctgcaatctcagtggaagtgttcacaatattatgccctggtaggaatgtaaacacgtctcgattatgttagttatgatgtctaaaggtatactgtccaactcttaagcactttatatgagaacactacaccaagatgaatcagcacattcagcacatgtgagaaatcaagtattttctttttatttttataaatcttagtggtttttttttttccggctcagactgtccaagacaaactccactactcgcatatccccagttgtttgtggtaggttgatgtaaatatggccacaatactgttagacttcacctccagatactttattgaacaactggtttacttctagcaccaaagggaagaagaagggggtccagtgatggtgatatcttagcttgcttctttgttcacaggcactattgaccacatgaacatacaggtactcaaatacaggaggcttctatttggctcctccaagggaggagttcctccaagggaaaaaaggggatggtattcttttaatcaaagaccatggtggatggatggtaagggcaacagaatgcaattcttttgtgtggtctgacgttaacacttcggaaaaaattctaagtcaagttgaaatcgccattctctcaagagagaaacatcaagagagaaacatcaagtgaccccactatgtgcctagagaaggaaaagggtcaagtcctatactaggttccttacttagggtagaacctactagtcccttgttaatcctttctgggattatacctgtgactaatttttttttttttaactatgtcttacgacaaagcacagtcctgggaaaccagtcaacttaattattgcttgtctttgagcattagagacatgttcatattgacttgctacataacactgtagcatgttttttcctgaaaatatcaagttggttgaatggaaagctcagcaaagtatgttctgtgtgcccactgtcccagcctgggactgacgggcatcagaggctttgctttaaaatatgggtaaccagttctggaagacttccaacgcaagcaagcttggtgttttagggtgtctccagatacataagcattgtctgcctccgctcagggtctcaggaaatacacagcccattggaaaccacccacgcctgagatttggaaacctgacggaaagggaggaaggtccaaatgagtcagcagcaggatgactcaccacaagcctggcaggctgcagagccagagaagggggcttccaacaaggaactcagagaagttgcagtcaccaccaagatctggcccttcggcaggtcagcaccaggactgccaggccctctgcagaccgtaattctcttcacatctataattaggaggccaaactccagctgccaacacccttggacacaatattccagtctccactgcccatctcatgtggttcagttctaatagcaggtcttcttcttcctttcattccttcctttattcagcaagcatttgttgagcatctactatgtgccaagcattgtgcttgggcaacaacagtccacaaagcagactccccacctgcccttatgaaactgtatctcctcctccccaacattatcaccaccccaccatttccacaatgaaaactgaattcagacagcattctccattctgccagagtaacctaggaagaagaacatggcagcagacggatcctcagggaggaggcagcagagtgacagatggtaaactgcatcaggccacatatttgcacctttcagatacagccaactcttccagggcctagactgagctggttggagctccccaaagcagcattcagttgcttttccttgatatccaccctggctacctatattccctttctttatataggcacaaccattcaccactacgctggattatctaacactccaattctataactcatccaggggacacacagttgagtctgatgaagcctaagaactgccaggtgctgagatattgtaactgggaattaagatttagccaaggacattgtgagactactgctgactctcttggaaaactggacatgctttgtagtgcaaatgttcactaaatgatggtggttcattgttacttactttggcctttgcaaaatattctgaacaattcaataagtagagtttcctagtttgtttgtttttttaagtcaaaggaacgctgtgaaattattgaaagatgatgggtgaagagggacctaggagaaggcaaaatgaagaccattaaagagccccattgaagactgtatctctaaaaatgccttaatgcctggcacatattccatatttgcatatgaactactattcttttccaaactgtcaacttctgtttgaaatcattgtgggggatagtttggaaatatggtggcataaaatccaagcagtcaagagctaaaccagcaaaaaagaggaatgacttcattcacaggctttggcttctttattatgtggtggtcctctgtgatctcccatggttaacttcaactgtgctgtactttcttcccaagatgcatgtacggaatgttacctcctagaaatccagaaatagttgtggtgagttttgtttgataggaagcaagaggtggggaagtaaaggatacatatttttatgtctaaggactgtaaactgttgatgcttgacttttccttttacctacacctgtgctacacctttattaaaaggctgacacccagacttgtgcaggaaaggcactcttgggaatgaacagaaatgaataattatgagactttccatggtgccaaggggattcgcttatttccactccagaactgaatatgcagagcattgccagtcctgctagtgtgaaaagcaaattacttcaatagtttgagacgtacctaggggaagagagatcctatcaactttctttgcgggtagtggcgtaccttcaaatccttgctcaggctctgtgaggtgtgcacccgcagatttcacttctacctccggaatcctggcaaacaagccaggaatctctccagagtcacttcgcagttgctcagaaagaggctaacggtccttagtcctgaggaaattctgccatttttaacattcaaggagaaaaggcgtatcagctgtacgtgagaatatcaactaaggataaaaacctctaatggatcccccttgttacttgcaaatgctttctttgtctgcaaatgtcacataattgattttgcataggaacaacaaaagtctcaaggaattgaagtttttaataccccccttagcactttactttcaccaaaacctcctctttctagtacatgagcagttttgttctttgtgatgttttcctctacattctttagaataattttgttacttgacaactagacttgtcaatcgtcttagactcttttgaaaaatctaatctttgaaaatgacacaaattcctttattgctttaaggctgttttcattcacccctcccacagccccctccactgcacttttttgttttttgcggggatttttttctttcatctctcttcttttaacatgatgctgtgggtagttttcaagaattctataaagaactcaacaaaaagtgtatttgttacctttttccccagtcctaatatttcttcttaagcttgatcttggtgaaaatttgttgcccaaaaagcagtgaggtagggattaataacgctgaccagtcatctggaagattaattaatcccagtgcagccctgagatcaggaaggaggacaggccaggagatgtttctactccaggcaccactaaggactctatttcaaaggcagatcctgctccttagtctttttagatctgaatctaatcctgaatccagaaaattatcctatgaattctggtttatcaacgcacatgattcctggcaccattgcatagcttcaaggtaaaagagagccttgtttccattattttgctatggtggcttttgggaagacagagagcattcttttgaaagcgggaaacttaaggaaaagttggccaagtacacaggaaagttctaccacaccttaatataaagaacaaaatagatgcttctcatttggggaaagtagctaaaaggacagtaacagtactgatggccttctgtccatcaccttagctccacagggaaaattctgggtgagagcaagcactgtttctaaaatctaaattttttcaggtgaaatttaccagcaacaaatgtaaaagggaactgccatcaggcatagggctagttttatacttcctgcctaacggcaaaatgactgagacatgacttccatggaacagcagaaaatggatgccaaagatatcataaaactgaagttcaatttgccacgtaaggctatttaaaaaccctgatgcactccaagcattcttccttcagagctttcctcctacaattatatcaagcacaagaaggcaccaacagcatatacttaatgtaatgtaaaagtaaacatatgacttgtgttgaattaccattggctggttgacataataagtgtcatagatatttagacattatttctgatcatacaaaattattttcataatttcatattcttttcaattttcataaattcttttcaattttcataaattggagccaatggtttgggaggttttttgtttgtttgtttgtttggttggttggttatttgttgcgttgttgttttccaaacctcagatattttgagaagccttagaaaggctcttaagcccaggcctttttgcctcatgtgcagagtattaataacagccctactgatgtgaacctcaaggaaggaggctattaaattaatccatcactggagatactaatacctcaaacaattaaaacactgaagactctgctggttgccttccacattgtttataacctttttaaattcagtcatttggatcaagagaaataaatggttggtttcatatggcttttgccacattagcacagacacatctaccaaatgtagaagtccagactgagtggatgcatgagagcaaacagatctgtaatatgattgcatttaatcaccgccaccgcccaaattctgtaaaaatatattcaaatatgtaatgctaaaaaaaatgcaaaacaggagactcttactagatcatgctttcacttagggtaggataactgatcttgaagataacttcatagaaaaatttgctggctgaaatgccccaggcacctcagggttggtcctgtcctattccacaaactgatcatggtcttctcccatcttctcagaggagttagccttgcctctaaaatagcaaaaatgccatgaaattatctttctaacctctatagagacttcttatccaagtataattccaaaaatatgatttgagtttgagcatgcctgtggtccttgattctatgatccctaaatcagttctacttttctcactctctctctctctctctctctctctctctctctctctctatatatatatatatatatatatatatatatatatatatatatatatatatatataatctccagtattttttttcaaccacaatttagtgcatttagcgtgttaatgttgggaactgcctgctggctctatgtctacttttgacaatgcttatgtccagataccatacagtgcatttgagagaggaccaaggttatgttttcccacataccagctgctgcagaggtttcgaagtgcctgacccagttctttttcacatcaaaaaggccttcggttgcagatggaaaccttaaagatgaatgtagagtggggtaggaaattcatgtttgaatgctgaaacccttgtcttttttagttttattcaattcccacctcagatcaccagaaagggaggcctctttggtgggtgttggccctcactgggggtaaaaggggttctatttgcttaaactcatgctcgtaggcaattttagggctctctggggctctgaatggtttctccaccaccacctttcttccaccaatagaaagatgttgtccttacaaggccatgtctgagaagaccatggtttaactagtaaaagaagctcacattgcagagagctgaataataccaggctcttgactttgtactgtgtgtcacagtcatgattgggacagaaggaaccagtctgctgaaaagtcaagccagcacaaatatttggaaactaattccactgaaactcttgtatcattaaatcatactattgtagtttcagttaatgtaattatttaatatcaggcactaaattaaatagctttgcacaaaattttagaaaagatcaatcttgtaacaaaatttaatattaacagtaaaaagcacctgagaaaagaaaatatcgatctgttgtggtttatatacttgagttattcttagaactttgtggagattgacactgctctgataaaaaggaagaaaatgttttgaattcaagaatgctagactaatagaaatagcccagtctctctgggccactgctcttcagtatggggtgctctctgtccctccttagcagatggggttgtttggaggctaaaatgagctaagggaagaagctatactgtgcctccaatatcccttatgtgtaattagagtgccgatctgggctgcaggctttattcaatgattcctttcccttacttgaggctttctctgtagagctgttatttctgagcctccattctgggatttacaaaggaaacagaatgatgaagaagtaacaatcagcattttacacacacgcacacacacacacacacacacactttttctaatatatgaggctagtctgcagagtccacagcccaggtgtccatttatttccacttcttcacctcccaaagatttagagctccatgatctaccaaggtgaagactgccaatcccaggttggagaagacccgtcagagtgtggaggtgccaaactgagaggcacaggaagcagacagccacatcaaacttttttgatgaactactgcaaattggtgtttgttctaggaaagaaagaggaagaaaaattacacaatgtgaattgaacataagagcagtgctctttacagagtatgagaagggaggatgggggagaatctgattaaaaaaatgattcattccttcacagacactaacaaacatggctaaaaagcacatgtcagaacacagaagcctaggtagatggttgacatttttataacttccttaagtgagtagttaaaccagcagtcttaattctgttggtcttccaagagtgtttaattacataagtattacctgtattcatttcccacaactgttgggtttttctttctttttttttttttttttttttttcctctgtgcatcctagaaaaactcccaggactagacttaggaggaggcaatcaagttatgtggtaaaacaagagtgccttttctgttggatatccactttagtttcctggcttccagggcataagatgtttagaaactttttttctctaaacataagaattattgtgtaccacaattttgaaccaccgatttccatatcttcagcagctatcaacttgccaattccctttgggtctcctttgtatattcttatgtttccttctgtttccagttgtcctcaaaaagagttgaggggggcatgactcttataaaatggataaaaatgaaactgtacagatgtttgcctcccttgtatctgtgagcatgatctctatcaggctggaaaatctgctttatcattttgtatatttgactattttgtattcagcattacttgactccttatgtgcatggcaatgtattaaaatgtgggatttctattaccgtgtaa

SEQ ID NO: 748 - DNA, 579 bp, homo sapiens
agtgtcaatccatatctacaaggacagagactggataatgttgttgcaaagaagtctgtcccccatttttcagatgaggataaggatccagagtaaagagaagatgctagacgaaaacccacattacctgttaggcctcagcatggcttatgtgcacgtgtaaatggagtccctgtgaatgacagcatgtttcttacatagataattatggatacaaagcagctgtatgtagatagtgtattgtcttcacaccgatgattctgctttttgctaaattagaataagagcttttttgtttcttgggtttttaaaatgtgaatctgcaatgatcataaaaattaaaatgtgaatgtcaacaataaaaagcaagactatgaaaggctcagatttcttgcagtttaaaatggtgtctgaggttgtactattttggccaagtctgtagaaagctgtcatttgattttgattatgtagttcatccagcccttgggcattgttatacaccagtaaagaaggctgtactcaagaggaggagctgacacatttcacttggctgcgtcttaataaacatgaatgcaagca

SEQ ID NO: 749 - DNA, 1212 bp, homo sapiens
gccgtccccagtgctgctgagctacggtgtccccttcgacgtggccaggaacgcgctccggctcagcgtgggccgcagcaccaccagggccgaggtggacctcgtcgtgcaggacctgaagcaggccgtggcgcagctggaggaccaggcctagcactggggccgccttccccaccccgcttctgggaagcccgtggcagggcacagggttgtccctccagttccctcctgagggctgtgccaggatgactgtctcatgccccctctgcattttgtcctggagtgccagcgagtgtgcacccccagtttccttccctggacccctgcagagctcacagggcccaggacaccaacgccgcataggactgcccacatgggaccgcccacataggaccgcccacataggaccgcccacatgggaccgcccacatgggaccgcccacatgggaccgcccacatagaaccgtcctccagtggtgaagcggaaacacttagctttatccaccctccccactgggaactgggcacgcctgttgtgagtgccctttcctggaaggtgtttttatctggaagatagaatccaagtatttataactcattgtcagccaaatgctatcatgaacgtaggaaacttgatttttttgttttgatcatggcctctacatgcacctttccagagtggtctcttctcaggccctttttagtccctttccaaagctgcccccaccaccctgctcctctggcctcagtgcacagtggcccccagcctcggccaggcctgctctgctcagcgcccaccgcccaccacccctcctgcttccccgagcctgaccctgttccgcccactggcaatcagggctgcgcacttccctgtccacggtccccaggccttcctgtcttgtcccttttgatcattattaactcagggtttcagctccaacctcgctgagttggtgcagctccaggtcattcctggggtgggaatcggatcatccctgactcagcttttaccttaattttatttgcagaggattcttttctcaaaatgctctggcatttggacacacatcacatgtcgatatttgcataggagtcattttcagtggaataacatttttaatgtgtggttttacggttcaaggaactacttgatgattttgaggaaacacttgccagaaactaaattaacgaataaaagatttcagtgcccga

SEQ ID NO: 750 - DNA, 100 bp, homo sapiens
actcgccgcagcctgcgcgccttctccagtccgcggtgccatggcccccgcccgtctgttcgcgctgctgctgttcttcgtaggcggagtcgccgagtcg

SEQ ID NO: 751 - DNA, 139 bp, homo sapiens
atccgagagactgaggtcatcgacccccaggacctcctagaaggccgatacttctccggagccctaccagacgatgaggatgtagtggggcccgggcaggaatctgatgactttgagctgtctggctctggagatctgg

SEQ ID NO: 752 - DNA, 199 bp, homo sapiens
gtgcctctagataaccatatccctgagagggcagggtctgggagccaagtccccaccgaacccaagaaactagaggagaatgaggttatccccaagagaatctcacccgttgaagagagtgaggatgtgtccaacaaggtgtcaatgtccagcactgtgcagggcagcaacatctttgagagaacggaggtcctggcag

SEQ ID NO: 753 - DNA, 106 bp, homo sapiens
ctgggaaggggcttgtggaggcagctcacttctgctatctcatggctcacgtgccctttggccactacaccgtgaagacagaccatctggtcttgctgggcagcag

SEQ ID NO: 754 - DNA, 80 bp, homo sapiens
catatcagatggacagatgtatcattttggggtccaccattcaacccactacaaggagttttttaaacaaaaataggaaa

SEQ ID NO: 755 - DNA, 114 bp, homo sapiens
cgagtggcatcgtgttccagtgggggactggtttggcatcatgtggacgacggttgtgccctgggcagactcttccattgtttttcacagcaaaggaaccaagcagagtgacag

SEQ ID NO: 756 - DNA, 148 bp, homo sapiens
gtgtatgtttggggaagcaacaagcatgggcaactggctaatgaggctgctttccttcctgtgccccagaaaatagaagcacattgtttccagaatgaaaaggtcactgccatctggagtggatggacacacctggttgctcagacag

SEQ ID NO: 757 - DNA, 225 bp, homo sapiens
ttggtaaaatattaaagaagatcaacaaggccatcgtctccaagaagaataaagacattgtgacagtggctaacgccgtgtttgttaagaatgcctctgaaattgaagtgccttttgttacaaggaacaaagatgtgttccagtgtgaggtccggaatgtgaactttgaggatccagcctctgcctgtgattccatcaatgcatgggttaaaaatgaaaccaggg

SEQ ID NO: 758 - DNA, 198 bp, homo sapiens
atatgattgacaatctgctgtccccagatcttattgatggtgtgctcaccagactggtcctcgtcaacgcagtgtatttcaagggtctgtggaaatcacggttccaacccgagaacacaaagaaacgcactttcgtggcagccgacgggaaatcctatcaagtgccaatgctggcccagctctccgtgttccggtgtg

SEQ ID NO: 759 - DNA, 110 bp, homo sapiens
tgacccagaccacagatccatcacggcagctggaaggggggaacctggccctggccatcctgctgcctctaggcttggtcattgtcctcggcagtggcgtttacatctac

SEQ ID NO: 760 - DNA, 118 bp, homo sapiens
tgacccagaccacagatccatcacggcagctggaaggggggaacctggccctggccatcctgctgcctctaggcttggtcattgtcctcggcagtggcgtttacatctactacaccaa

SEQ ID NO: 761 - DNA, 91 bp, homo sapiens
gttctttttcttcaggatcaggcacagagactgactgaatggctccaattatcaggatttgaaaacccagtatcagaatctaccactttgt

SEQ ID NO: 762 - DNA, 593 bp, homo sapiens
atgttaattattcatactgcatgactgaggattttggaggcagagagagattcatctgcaatatttggaacaccaatggaggtctacgtcaacacagaatttatacagcagctggtgctagtcagagctaatgacagaatttcagtttaataaaaagacccccaactgagcacaccatcttgaaaaaagtatacttatcaaacagctttcaatcagttcaagagagacaccttaattggggagaggaagaattgcagagtagtttgtaatcatgccaattccagatcaataactgcatgtctgttctttggtagaaatagcttttgctttatattaagtaatcacatatatattctctctatttggataaggaaaccttcgctttatttgacaatgtataatgatatactcttctaattcacctctgtgtcttcacaataaacatgagtaaaatttagacaagtgatggtaaaggtcaatataattatttatttttaaaataaattttgtatctaacaggaaagcagttcttatgaaatttttatattttcaaaaattgttttgttcaaataaaattttatgagtaaagttaa

SEQ ID NO: 763 - DNA, 4094 bp, homo sapiens
gccactgtgtaaatagaactgcctaatgttgagagtggtttttagcattaggtttagcaagggggagatccgtgggttgtgcgtcagctttgggtgaattttgtttctaccctgtcacggggaaagttcgggttgagtccaggagtgcacactgctgctgccacccaatgcgctacatatcacttttttttgttttgttttgttttgtttttaaaagatcattttatcttaaaaaggaaagctgatccaagtaaacacgaaagtatttgacacaccccacagattttacatgtgtgtaaatgtttcactttaaaatctctatgacagatacacaggaaacatgagatggtttctgctaatgagtggcccttgagtacacacttagatgctgtctgccctgtaaatttggatctggtgccccagggcagtcaactcttctagcacaggctgaaaacacgtgtgtgtcaactgaggttcacaccacttggggaatgagcctgttttctttccaggtcaggccctggtgtgagatcaattttagggctcctaattggagcacagaaatgtatttggtcaaatttcattgaaggtgatttcttcctttttctgttgtactttatggaaaaaccaagatggaacctgaaagttaatgtatccttgcttttagcaagagactcagtttcttataactagtcctaaggacatatgccgcaattgagtaattttacttttaccctatgaaaataacatggtgctgcactataatatactctcggaatcagtgtgttagttacagctatacagtgaagggggataactgtttctaaatttctttaaggtgatgccaaaaaatggattaaaaaatctgatcagatttaatgttaccagatttagtgcattatttaatgctattggatcttttggataattcttggcttaatttcttagctacttgaaggttaatttgcaagacttttaaaaccttagaaaagttttaaggttgcaaagttatcaacactggggcagagggtggagaggccaatgcgggtagaaggaggcagttatgtttatattgaaggtgaaatttttctttcatttagaatggaaaacatccccaaatgtatcattataaactagtcagccttgactacacaaaattgacctttaagttgcttgagaaaaacacaatgcaaatcgttcagaagggtcaacatcctttggtgctaaaatcttgtgtatgttttcagaatggctttttctgtatgttatagaataatcactaaaggaaaggtagttgaatttaaagtcatgaagcaagactctttaattcagttattttaaacaagaattaaaaccccaaacatccttggcaggctttgaagcacacagaattttctagtatttcttattaaatacaccaataataaacatatacttagtttacctgatgagtttagacataattctatataatgttcgcttatattcatttattaactagcaacacttgtgcaagaacaaggtattctgcttgaaaacactgatttttaagttcctgttgcccgattgtaaagaaaatcatctgacacagaagcctggattttgctctcctaacactggtgttttccttgagcctctaattcaaataaaacaaactgatacaccttacagccttattcatctgtgtctggataactcacttttcatctattgaataagaaaaagtgtttaaacttaataaaataaaaaattgtgcaccaattactacagatccttgaaacagtaagccttggattttagtcaagtaatccagtttttttttaatttaaaaaaaaccatcaccttttaaagaactttaaaaacacctgtgatgccaatgtggacattgccgctgccatgtctttacacgcatgtgtcgtatagctctgtcatcgagttgaggaagtccatggtttgcaaataagggtgggggaatcaaaatttcaagatggaaaaataaaacaaatagtaaaacccatttacatgtaggaaagaaaagttgagcttcaacccaatgtgccactttgatataaaacaagtatgatatatatataaactgaagatataaaacaagtatgatcttgcctttgaaatcataaatgtttggggtacctgttctttgccagttaagatacatatcttattatctttgtttttttcaagtctatgctcctgtttgaagcttttcctgtaatttaggttgtctgtgaaatacctataacatataattcctatagagtatgccacattttttttctaactcatttcaaatgaaattctctcagattctagtttttgagcttgtccactagatctgaaaataaagcatcctttcctgagtccacttgaactaattgtgaatttgttacttaatttactggcatcttgggaaacaagttttgctgtggcaggaaggctgttttgagagtgagcgttgagtctactctggtttgtggatgacattgcattagggttatttcctgtattaccagtgcccccttgtggcaatatactttatgacttggaatgcaaacaccacttttaaaagcctgttttcaagtttttgaaaggcatttgttttctgttgtttgccaataatctgaaagtatcatgtgaaaggaattattttaaaagcattttaaacctttcccaaaggtaaaatgtgctcagattataggcacttgtaattctaaccttgtatccatttgatcaaagaaaatttgtagaacatttttacctctcatgatcacaatcattagtgtctccctttataacgacctatgttttgtttactttgaaacaccaaatttgttgtcttggttttttttacatgacagaactcatgcagtttcttttttagttgaaagttcacatcttgccccttgaatagtttgaacatttctttcttaattcttttttgtttttgttttgcactgtaaaactgatgaaaactttaagccaatcttaatgcactagcctctgtagtgtaaggaagtgtgagaaggagtttcttaatgagtttacacgtttaaatcaagcatattataagttatatgccatgtgttgaaggcttttctatttatatatataaagatttttagtatttgtttttagtgtccttgtgttgcaataatttaactcctttgactcagttgctgaaaatatttcttctattaagaagtgctcttgacttcaacacccaaacactgaaaatcatgtcaatgttacccaaagataagtttttatacagatacacaccttataagttctgatttattttcttactcattatgctgtgttctaaataaatcatgaatgagaagagtgctttattgtgaaattatttaaaactgtcctttaaaagaaaaagaggaaacgatgaacaaaaactaatctaattgccaagttagaattcattatttaatttacctcctatgcaatgattaatgctgcaaaatgtatggttatgttaccgtatattcacaaaagaaatattattacaaggtttcagaggtagccaattgcattcttttggaaatttactgtactgtttcaatgtgttaagtgccttgttgtaaagtaaaattttaagtctagattcattattttcctgacatatatttttcattatgatatctactgtatgctattgtgatagttttatgaaatgcttacattttaatcaaatatgtaaatttcagaagctcttttttcctacccaccagtaccttaatcattggtttatcacattggattcaaattcaggttccttttttgataaagaggaaatttgttttccatgcttgtgattttgtgtttgtaaacatttcaggaggaatttaggagtgtaactatagtttatacttctataatgttgtcttacatttacatttttaagtgcctaattgttaaaactgatttatgtttctcttctaaagaggatatgtgtacttggtttctaatctctttggttttgctttttaaaaaatgcaagagcctatactttgtatttacctaataaaccacagtgacatcaacagtcttttcaaaattaa

SEQ ID NO: 764 - DNA, 85 bp, homo sapiens
gagatgccgcaggacctgaggagccccgcgcgaaccccgccttccgaggaggactcagcagaggcagagcgcctcaaaaccgaag

SEQ ID NO: 765 - DNA, 1075 bp, homo sapiens
ttggtactgatagagaagaagcagcagctgacggggcagttggcagatgccaaggagctgaaggagcacgtggaccgccgggagaagttggtgtttggcatggtctcccgctacctgcctcaggaccagctccaagattaccagcactttgtcaagatgaaatctgctctcatcattgaacagcgagagctggaggagaagatcaagctcggggaagagcaactcaaatgtctcagggagagtctactcctggggcccagcaatttctaattctaccagcactctgccacagcatccctgcccagccatgtgggaagtgctttcaatcttctttgttagcagtttctcagcaagtagatagcaattagcagtttgttccagccctctaccctggatgtctctcactaccccttccctagcagtggtcctaaccagctaggagaccctggggaagccacaagcttctacccaagggagctgcagcaaggtgtgatcttagaaccacactctccttcccacagttgccaagggcaagtacttgctgcacagagaaccaaggaagtgccttcattctgctttgtactaggacaccaaagacatcaagtactcatcacccacccatatcatcaacagcctctaaaggctcagagggaatctgccttgcagctctactctgccccagggcttgtggccagccatttctcacagagagctggctgccttgagggcattcacctggcaccagtttcagggcctcacccaagctttgcaggggaaagcacagagggaggaattacactgaaaaaaatgcaagcaaaggttgagtacccccaggtgccccttaggaaggaaccaggtttaaataggctctacccttacctttcccagcagcaagttcaggggaagaggcctactcttagccctggctagtgtgaccctcttcctgtcctaagactttggtcctaccacctcttgtttcatctttcctttacattgctgggggttaccgcaggtgcctaccccagggcttcaccatatgggccattaatagctctactaaaactgacttctagat

SEQ ID NO: 766 - DNA, 84 bp, homo sapiens
gcgagaagagggaagttggagagggaaaagttagcccagaaggaaagcattttctgcagatcagcctgaatccaccgtggctag

SEQ ID NO: 767 - DNA, 190 bp, homo sapiens
acaagcaccttgccctccagttcatagactgggtcctgagagggaccgctcaggtgatgttcatcaacaatcctctcagcggcctcatcatcttcatagggctgctgatccagaatccctggtggacaatcactgggggcctggggacagtggtctcgaccttaacagctctcgccttgggccaagacag

SEQ ID NO: 768 - DNA, 95 bp, homo sapiens
ccacaggccactacaacctcttcttccccacaacactggtagagcctgtgtcttcagtgcccaatatcacctggacagagatggaaatgcccctg

SEQ ID NO: 769 - DNA, 175 bp, homo sapiens
gtgggcgtgccaccaggcacctgggccttctgccttgccaccatcatcttcctgctcctgacgacaaacaacccagccatcttcagactcccactcagcaaagtcacctaccccgaggccaaccgcatctactacctgacagtgaaaagcggtgaagaagagaaggcccccagcg

SEQ ID NO: 770 - DNA, 50 bp, homo sapiens
cactgtttgctgcctacctgggtgctgccctggctaacatgttatctgtg

SEQ ID NO: 771 - DNA, 34 bp, homo sapiens
acaggacctgaatgaccgattgtttcttgcttac

SEQ ID NO: 772 - DNA, 819 bp, homo sapiens
gtttaggtttagccttcaacctgcaacccgccttaaccataattggcaaatacttctataggaagcgacccatggcaaatggattggccatggcaggaagtcctgttttcttaagttcattggctcctttcaatcagtacctttttaatacttttggctggaaaggaagcttcctgattttgggaagtctacttttgaatgcctgtgtggctggttccctcatgagaccccttggacccaatcaaaccacttctaagtctaaaaataagactggcaaaacagaagatgattcaagcccaaagaaaatcaaaacgaagaaatcaacttgggaaaaagttaataagtatttagatttctccctttttaagcatagaggatttctgatatatctgtctggaaatgtcattatgttcctaggtttttttgcccccattatattcttggctccatatgctaaagaccaaggaattgatgagtactcggcagcttttctgctatctgttatggctttcgttgatatgtttgctaggccttctgtaggattaattgcaaactccaaatatattcgacctcgaattcagtacttcttcagttttgcaatcatgttcaatggagtgtgtcacctcttgtgcccactggcacaggactacacaagcctggtattatatgctgtattttttggccttggatttgggagtgttagcagtgttctctttgaaactctcatggacctcgtgggtgcaccaagattttccagtgccgtcggacttgtcacaattgtggagtgtggcccagttcttcttggccctcctcttgcag

SEQ ID NO: 773 - DNA, 996 bp, homo sapiens
gttaaaaaaagtccttcatgaaaaagaaagatcttaagcagcatgatggattcagaagctcatgaaaagaggccaccaatactaacatcttcaaaacaagatatatcacctcatattacaaatgttggtgagatgaagcattacttgtgtggctgctgtgcagccttcaacaatgtcgcaatcacatttcccattcagaaggtcctctttcgacaacagctgtatggcatcaaaacccgggatgcaatacttcagttgagaagggatggatttcgaaatttgtatcgtggaatccttcccccattgatgcagaagacaactacgcttgcacttatgtttggtctgtatgaggatttatcctgccttctccacaagcatgtcagtgctccagagtttgcaaccagtggcgtggcggcagtgcttgcagggacaacagaagcaattttcactccactggaaagagttcagacattgcttcaagaccacaagcatcatgacaaatttaccaacacttaccaggctttcaaggcactgaaatgtcatggaattggagagtattatcgaggcttggtgcccattcttttccggaatggactcagcaatgtcttgtttttcggccttcgaggtcccattaaggagcatctgcctaccgcaacgactcacagtgctcatctggtcaatgattttatctgtggaggtctattgggtgccatgttgggattcttgttttttccaattaatgttgtaaaaactcgcatacagtctcagattggtggggaatttcagtctttccccaaggttttccaaaaaatctggctggaacgggacagaaaactgataaatcttttcagaggtgcccatctgaattaccatcggtccctcatctcttggggcataatcaatgcaacttatgagttcttgttaaaggttatatgaaaaaaccatcagttaagtgccatttatcaactgaatagaccttctaagaagaatgcagtt

SEQ ID NO: 774 - DNA, 204 bp, homo sapiens
atttgcttggaacggtagaagaaatccttccaagcctctcagaaaatatcagtgtttgggggatgaaagattctgttccacaaggtgtaatttcactcaaagaaaaactgagcacctcacctgatgagcccgtgccacgcagccaccatgttgtctcactcctcaagtctacttgtctttacatttttacctctggaacaacag

SEQ ID NO: 775 - DNA, 195 bp, homo sapiens
agagaaggagaaaaggatcataaggtgcgtttggcaattggaaatggcatacggagtgatgtatggagagaatttttagacagatttggaaatataaaggtgtgtgaactttatgcagctaccgaatcaagcatatctttcatgaactacactgggagaattggagcaattgggagaacaaatttgttttacaaa

SEQ ID NO: 776 - DNA, 199 bp, homo sapiens
gagaacctggacttctcatttctcgagtgaatgcaaaaaatcccttctttggctatgctgggccttataagcacacaaaagacaaattgctttgtgatgtttttaagaagggagatgtttaccttaatactggagacttaatagtccaggatcaggacaatttcctttatttttgggaccgtactggagacactttcag

SEQ ID NO: 777 - DNA, 131 bp, homo sapiens
gttatgaaggaagagcaggaatggcttctattattttaaaaccaaatacatctttagatttggaaaaagtttatgaacaagttgtaacatttctaccagcttatgcttgtccacgatttttaagaattcag

SEQ ID NO: 778 - DNA, 343 bp, homo sapiens
ctttcatatgctttcttaggaagagtgagaggggggtatatgattctttatgaaatggggaaagggagctaacattaattatgcatgtactatatttccttaatatgagagataattttttaattgcataagaattttaatttcttttaattgatataaacagtagttgattattctttttatctatttggagattcagtgcataactaagtattttccttaatactaaagattttaaataataaatagtggctagcggtttggacaatcactaaaaatgtactttctaataagtaaaatttctaattttgaataaaagattaaattttactgaaatatttta

SEQ ID NO: 779 - DNA, 32 bp, homo sapiens
gtagggcctgctcttttctaccatggcagccc

SEQ ID NO: 780 - DNA, 151 bp, homo sapiens
gtatatgatgggcctccccatgtggatccttagtgctgtggcagagcccttgttattgtgctgggattttccctccagctcccggccggaagctgggctcacgtgggagctcagtgccctcctgctacagatctgtctcttccttacaatg

SEQ ID NO: 781 - DNA, 139 bp, homo sapiens
gtcaccagtcatttatggctccttcttgtagtgttttgcactgccctgccgcctgtacctgtagcaacaatatcgtagactgtcgtgggaaaggtctcactgagatccccacaaatcttccagagaccatcacagaaat

SEQ ID NO: 782 - DNA, 72 bp, homo sapiens
acgtttggaacagaacacaatcaaagtcatccctcctggagctttctcaccatataaaaagcttagacgaat

SEQ ID NO: 783 - DNA, 504 bp, homo sapiens
gagcccttatattatttggagctatcgcattgaatttggtgccttctagtatgctcttaagacccatccatatcaaaagtgagaacaattctggtattaaagataaaggcagcagtttgtctgcacatggtccagaggcacatgcaacagaaacacactgccatgagacagaagagtctaccatcaaggacagtactacgcagaaggctggactacctagcaaaaatttaacagtctcacaaaatcaaagtgaagagttctacaatgggcctaacaggaacagactgttattaaagagtgatgaagaaagtgataaggttatttcgtggagctgcaaacaactgtttgacatttctctctttagaaatcctttcttctacatatttacttggtcttttctcctcagtcagttagcatacttcatccctacctttcacctggtagccagagccaaaacactggggattgacatcatggatgcctcttaccttgtttctgtagcag

SEQ ID NO: 784 - DNA, 34 bp, homo sapiens
atgaggccatgaatgaggtcgggtcagcccaggc

SEQ ID NO: 785 - DNA, 85 bp, homo sapiens
aacaaagaacaacagtccagggccaagtttactttttgcatacacagactggagttagcacgtggcacgaccccaggataccaag

SEQ ID NO: 786 - DNA, 187 bp, homo sapiens
tcttgaagccggtagtggagttactgagtaatccagattacattaaccaaatgctgcttgcccagctggcgtacagagagcaaatgaatgagcatcacaagagagcctacacctatgccccctcttacgaggacttcatcaagctcattaacagcaactctgatgtggagttcttgaagcaactaag

SEQ ID NO: 787 - DNA, 1145 bp, homo sapiens
gcatcgccctgctctgatccgccagacgcggagcccgggcccggggcagcgaaagggccgccgcagcagaagccctgtatgctgccggtgcgctgcgtcccggcccccgccacgctccgcctccgggcggaggagcccggcctgcgccggcagctgtcggaggagccgagcccgcggagctcccctctgctgctgaggcggctctcggtggaagagtccggtctgggcctcggcctgggcccgggccgctccccgcacctgaggcgcctgtcgcgcgccggcccgcgtctgctgagccctgacgccgaggagttgcccgccgcgccgccgccgtccgcggtgcccctggagccgtccgagcacgagtggctcgtgcggactgccgggggccgctggacccaccagctgcacgggctgctgctgcgcgaccgcggcttggcggccaagcgcgacttcatgtctggcttcacggccctgcattgggccgccaagagcggcgacggcgagatggcgctgcagctggtggaggtcgcgcggcgcagtggcgcaccagtcgacgtgaacgcacgctcccacggcggctacacgccgctgcacctggctgcactgcacggccacgaggacgctgccgtgctgctggtggtgcgtctgggtgcccaggtgcacgtgcgtgatcacagcgggcgtcgcgcctaccagtacctgcggcccggctcctcgtacgcgctgcgccgccttcttggcgatccaggcctgcgaggcaccacggagccagatgcgaccggtggtggaagtggcagtcttgctgccaggcgccccgtacaggtggccgccaccatcctcagttccaccaccagtgcatttctgggcgtcctggctgacgacctcatgctccaggacctggcccgaggcttgaagaagtcgagctccttcagcaagttcttgagcgcctcgcccatggctccacgtaaaaagacaaagatccgcggtggtctgccagccttctcagaaatctctcgtcgacctactccggggcctttagctggtctagtgcccagttttcctccaacaacctgaaggtccctggggctaccacctggttgatctatcgtggcctgctcgtcgcagtccaaacccgcccaaga

SEQ ID NO: 788 - DNA, 72 bp, homo sapiens
gtcagggctaaggccatggccccgcccccaccgccactggctgccagcaccccgctcctccatggcgagttt

SEQ ID NO: 789 - DNA, 346 bp, homo sapiens
aatgctttgtgtaggctatttcttttcatccttaacacaaatggaagggaggtattactgtaaatttgcagatggggaaactgaagtcatcaaggttaaataacttgctgaattcccaccaccagtggatcacaaagctgtgtgacactggacatgacttcccaaaagaaccccatcttgccatcctatgatctgtagatgaaagtagggtttgctgatggccctgagaaaatcttaacaaactggatgaggctcaaagacttttccagggatatggctccagaactgtgaccttccctttatgggtccagcatgacatcgtcctgctgctccatatggcatcaat

SEQ ID NO: 790 - DNA, 3547 bp, homo sapiens
aagaggagcaggaaacctcaggggattttggcagtggtgggtccgtggtcctgctggatgacctagaatatgaacgggagctgggaccaaaggacaaagaggggaagctgagggtgcacacccgagccgtgacagaggatgatgaggatgaggatgatgacaaagaggatgaggtcgggtacatatggtagtgcccacaagaaagaggacacaagttttgcacaaaattgcaagtcacttcctattcctgcatttgtatctaagactccaaggcaccaaggtctcttctccattgttgctctctatacccgacctaaggtttggaagacaactgcttgttcccagaggattctgattttgcatatgtttgtatgggagaaagggtgttgtgttttttttttgttgttgtttattttttggatagggaagtcattggcttaattagagcctccttcctttctgtgagatttttccaacaagcatgtgatttacgtggaattctgacagtgcagggagccccaaccctcttaaatgtcaaagaccctttttgattacccacactggtggttattacagcatggttcccagccttacagtgtctaagtgcttctcttgtgtcctgtagatgttgtgaaaaagaaaaaaacaaaaaatacaccacactgtactttttccccctgcccccgttactgccggtgattattattaaaaattagtttttttcacatcattatatctggcttcctataaacaacagccttaattcagtcaagactccctttggggaattcattttattaaaaattggtgtctggatacttccctgtacatgcataaatatgcatgcatgtacagaaagactgtatgtgtgtgccttgcacacacacccatacctctcagaaaaagtgtttgggtatcttaaaaactcgaaaaacaatgataaatttctcagcttgtccagacctggaacaaaatttctggaataagaaatttgtattaaagtccttttttgcactaacagttggctcttgtagcctgcaggctgaggaagtctcttctctgtgcatcagcagagttactgaaagcctctgattgagaaaaaacctccgtctgcctaaatcacttttctcgcagaagccatgcgactcccacacgacacgggcagcttcacaagccatctctttcatttctgcttgaagccccttggctgcagcaatcctgtctgccataggtttcttccttccttacctactcaagggctttttctaaggcatgcacacatatctcctgttctctgagagtaccatggtgttccttaaaagaagaaaatttctaattctgaactcaatgttttgcttttactccctttctactgacaaatcatgataagggcacaaaagctgtacagatttttttttttaaccactcaatcccaaatggaggcctacaaagaacatcgtaataacacatggaagcaaaccctgggtttttaagagcaaattctgtccccccctcactcccccaagtgacaagatactaatgaagaaagttcttcaccatagtgtttgttttaactaaactcattggagtctagttccaaatttggtagggtcatcatctctacattccttaggatttctctccctatcaagctggcccagatacaagtaccaaacagtagtctctgaagttcccccatttccttcagtaccagtctataagctactgtccgccactgattttcatttatcagggtgtcctaatcagaatcagccacccaagcaagcctctctggcccacatatctatctcttgccttcccccatgaacttcagcctgtccacacaaaagccacataaactcaagcaagaaatatgttcagccaaaacatgattatagtggcagctgaccaatcccccatcccaaattgaccatttagatgtaccaactcaccttaaattagcatgttccaatccagtcggcattgcctgaatacagtagcatcatacctatagttggtcttagataagaaatgaactacttgatatagcaaagtcctttggcttcgtaaataaccctgaggttttgtacttactttccccataggaagacagaccataggcaaactctgttttgggatctcaactccatcacctttgtttcaatattttttttctctcttgaacaaaactgagataatttagaaaacaggtgcttaattgcaataaaattactatgaagtatattaaaaatcacgacattgtaaaatctcactttagatcatcaaagaaaaccattgttactatctcctttgagcttaggaaaatgtacaagagaacaaattaaaattgaaaaattgatttcacttagaaaaacttctaggaacagggtgaaccactgattttaatttgcctaattatcttatgacaagtatcaaattaagatgacacttaaagatccttagcattaacttaatgatggagaagagtgctcaatagacagttcccagtaaggtaatgagatgccattttcagagacattctaagaagatattttgattcattaaaacattaaataaaaagccctcctcagattggaacccccaaatcgatggagccacattaataatacttttcatgcctcactttgacatgacagcattcgatttttttaaagatctttaatactttccatgagtactaaagattgtaatgagttaccttatccttagaagtagaatgtttgctttcttcttcttggaaatagtcctccaaaaagtccacttgtaccagtgacaagaagtcacttgtatagtgaccaagtacacataaaatactgattataaaaatattgtaataaaccacttcctcatttgatacaggtattaagctgaatcctcactaattcacttttgaaaagttcttaatgaacagttttattcttattccatgaacagctcttttaatacaatttccttggtatccaacttaaatcccagaattttgttccaccatggttagttatttgccatatgaatgtattcttgtttctaccaatattctaggcatgagaataccttaatactgagttggagattttgtatctgtttctcctcctttacgcccattttcctacccacagcagcaaatgacaacgtgtctgtccaggtctgtccccctgctcatcccaggatgccactcacatttttttcttcttgttaccttgaccacgctgtacagtaacatccaagagcccattctacagtgggtggttttggtctttttataactttttctcaaagtcactgatgtttgttcctgttaaatgtatagcattgtaatgagagcccatcaaatcctgagtgtcagtttgttgtccctattgtagatgaaatagtgatgtagcaaaaacctagtaaattctgaatgcttttccacgtagacttatctggaatgtgaacacaactctttggttaatagtaaatgcttaactgtagtcctgagtaggtgcatttctgtctgtctcaataaattttactttgtctgcaa

SEQ ID NO: 791 - DNA, 117 bp, homo sapiens
gatccccaacaacagcctgccctacttccacaagcgcccgcaggcccgtatgctgctcctggcgctcttctgcgtggccgtcagcgtggtgtggggcgtcttccgcaacgaggacca

SEQ ID NO: 792 - DNA, 130 bp, homo sapiens
caacaagtggcccccacggatgatgagactgggaaggagctggtcttggctctctacgactatcaggagaagagtccccgagaggtcaccatgaagaagggagatatccttaccttactcaacagcacca

SEQ ID NO: 793 - DNA, 149 bp, homo sapiens
gattggtggaaagtggaagtgaacgatcgtcagggttttgtgccggctgcgtacgtgaagaaattggaccccgcccagtcagcctcccgggagaatctcctggaggagcaaggcagcatagcactgcggcaggagcagattgacaatca

SEQ ID NO: 794 - DNA, 34 bp, homo sapiens
gagcagattgacaatcagacacgcataactaagg

SEQ ID NO: 795 - DNA, 93 bp, homo sapiens
gcgcgcaatcgtcagacgaactcagtgtacggccgccacgcgctgcccagccacaggctccgagtgccggcgcgggccgcctgggtggtgcag

SEQ ID NO: 796 - DNA, 99 bp, homo sapiens
aattctcccaataatatgagcctgagtaatcaaccgggcactccaagggatgatggcgaaatggggggaaatttcttaaatccttttcagagtgagagt

SEQ ID NO: 797 - DNA, 1078 bp, homo sapiens
ggatcccaatcacccttcagtaccttatcattattatgaaccttttggacctgatgaatgtacaatgtacctctcccatgagcgaggacgcaagggcagtcatcaccgctttatcacagagaaacgagtctttaagaactgggcacggacattcaatattcacttttttcaaccagactggaaaccagaatcacttgctataaatcatcctgagaataaacctgtgttctaaggaatgagcatgccagactgtaatcccaggtattcactgcatcagacaccgagacactgaacttcctgagccaccagacaggaaagggtagcagaaaacagcttcactcctcaggaagtaccatggacagacgcctaccaggggtgacaaagcagtgcagttggattgtaaggaaaaattccggaattaatgcatcctaatgaatgttgtccccttcaatggtgttaccttaggagctgaacattcaattcagttacaccactatgactaaaaacagtttggatctcttagtattgcctttgaaactgcaacataagcaactcaacaatattagttgcattcctttatagacataccatgtcaaagacgtttttctatcaagttgtattctttcctgttctataacctttgtcatctgttagactctgtatgtgtgatttgtaaaaagcaggctgaaactatggacatgatttctgaagagcacatctccactgactttcataaagcaaatgtccaatatttatttattgagagttttttagtgcaatctgggccagtatttttatagattatgattatgtggtaatttatccttcctaactctttaatcctgaatgatggttggaaatggcctagaattaggttactctgttcacaatgctcattgttagcatgcaattggtatttgacttggaagtgttgtgttgtattttttgaacccctaggcttcaggaaaactgctcttttgtaaaaagaatagcgatgacattttctaatgtgcagaaatgttccaaaaggacaaaattgaaaaccaaaaactatgttattaaaacaaaaaaatgctaa

SEQ ID NO: 798 - DNA, 72 bp, homo sapiens
aaattgaggcaaaagaagcatgtgactggctccgtgctgccgggttcccgcaatacgctcagttatatgagg

SEQ ID NO: 799 - DNA, 56 bp, homo sapiens
tcaactttgaccactttgaaattttgcgagccattgggaaaggcagttttgggaag

SEQ ID NO: 800 - DNA, 98 bp, homo sapiens
cacctgagatgttcagctccagaaaaggagcaggctattcctttgctgttgactggtggtccctgggagtgacggcatatgaactgctgagaggccgg

SEQ ID NO: 801 - DNA, 117 bp, homo sapiens
agaccgtatcatattcgctccagtacttccagcaaggaaattgtacacacgtttgagacgactgttgtaacttacccttctgcctggtcacaggaaatggtgtcacttcttaaaaag

SEQ ID NO: 802 - DNA, 34 bp, homo sapiens
tgtcacttcttaaaaagctactcgaacctaatcc

SEQ ID NO: 803 - DNA, 129 bp, homo sapiens
aaaggcaggctgaattgtgatcctacctttgaacttgaggaaatgattttggagtccaaacctctacataagaaaaaaaagcgtctggcaaagaaggagaaggatatgaggaaatgcgattcttctcag

SEQ ID NO: 804 - DNA, 77 bp, homo sapiens
ttgtggaagatatcaataagcgcagagagccgctccccagcctggaggctgtgtatctcatcactccatccgagaag

SEQ ID NO: 805 - DNA, 104 bp, homo sapiens
cttgtccagatgccctgtttaatgaactggtaaaatcccgagcagccaaagtcatcaaaactctgacggaaatcaatattgcatttctcccgtatgaatcccag

SEQ ID NO: 806 - DNA, 65 bp, homo sapiens
cctgaatgagatgcgctgcgcctacgaggtgacccaggccaacggaaagtgggaggtgctgatag

SEQ ID NO: 807 - DNA, 34 bp, homo sapiens
gtgggaggtgctgataggatccacacacatcctc

SEQ ID NO: 808 - DNA, 110 bp, homo sapiens
gcatcacggaggaaaacctgaacaaactgatccagcacgcccagatacccccggaggatagtgagatcatcaccaacatggctcacctcggcgtgcccatcgtcaccgat

SEQ ID NO: 809 - DNA, 155 bp, homo sapiens
cgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgatag

SEQ ID NO: 810 - DNA, 206 bp, homo sapiens
cttttgaccagatcggtggttacgggcagctggaggcagcctacgcccaggccattccctccaggaccattgccaacaccacctgccacctgccacgtacagacgccatgcacatgtttcgagacccccacacaggggacctgccgtggaccgggatgacctttggcctgaccatcatggccacctggtactggtgcaccgaccag

SEQ ID NO: 811 - DNA, 34 bp, homo sapiens
taacatcatgcaggcagctgtggtacaatatgaa

SEQ ID NO: 812 - DNA, 87 bp, homo sapiens
gccttcgatgtggtggagaggagcttcctggagtccattgacgacgccttggctgagaaggcaagcctccagtcgcaattgccagag

SEQ ID NO: 813 - DNA, 161 bp, homo sapiens
tgatggagaagggagaaaaaggacctcatctacctgcagcaatgagtccctaagtgtgggaggaacctctgtcactcctcgccggatctcctggcggcagcgcattttcctcagggttgcttctcccatgaacaaatctccctcagcaatgcaacagcaag

SEQ ID NO: 814 - DNA, 156 bp, homo sapiens
gacacccagagacatgagacctaccaccagcaggggcagtgccaggtgctggtgcagcgctcgccctggctgatgatgcggatgggcatcctcggccgtgggctgcaggagtaccagctgccctaccagcgggtactgccgctgcccatcttcacc

SEQ ID NO: 815 - DNA, 142 bp, homo sapiens
actcccctggacccccaaaaggagaaaaaggagaggaaagagaaaaggcaaagaagaaggaaaaagggcttgagtgttcagactggaagccagaagcaggcctttctccaccaaggaaaaaacgagaagaccccaaaaccag

SEQ ID NO: 816 - DNA, 147 bp, homo sapiens
gaaatggccagtgatgaactgagggagttgaggaatgccatgacccaggaggccatccgtgagcaccagatggccaagactggcggcaccaccactgacctcttccagtgcagcaaatgcaagaagaagaactgcacctataaccag

SEQ ID NO: 817 - DNA, 278 bp, homo sapiens
tgcttgcattaaaggacttcctcatcctttttttcatgaaactgagcttgcttaatcagagatggagcaaactgactgcaaaccctaccagcctctaccaaaagtcaagcatgaaatggatctagcttacaccagttcttctgatgagagtgaagatggaagaaaaccaagacagtcatacaactccagggagaccctgcacgagtataaccaggagctgaggatgaattacaatagccagagtagaaagaggaaagaagtagaaaaatctactcaag

SEQ ID NO: 818 - DNA, 57 bp, homo sapiens
gtttcaaattctctcctgtttgttgtgacatggaggctcaagctgggtctactcaag

SEQ ID NO: 819 - DNA, 241 bp, homo sapiens
atgtgcagagcagcccacacaaccagttcaccttcagacccctcccaccgccacctccgcctcctcatgcctgcacctgtgccaggaagccaccccctgcagcggactctcttcagaggagatcaatgactacccgcagccagcccagcccagctgctccagctcccccaaccagcacgcaggattcagtccatctgcataacagctgggtcctgaacagcaacataccattggagaccag

SEQ ID NO: 820 - DNA, 239 bp, homo sapiens
gcatttcctgttcaaacatggatctggttcctctgcgatcttcagtgcagccagtcagaactaccctctgacatccaataccgtgtactcgccccctcccaggcctcttcctcgaagcaccttttcccgacctgcctttacctttaacaaaccttacaggtgctgcaactggaagtgcacagcattgagcgccactgcaatcacagtgactttggccttgttactagcctatgtgattg

SEQ ID NO: 821 - DNA, 200 bp, homo sapiens
tgtttcagaagggacgggcgatagacactggagaagttgacattggtgcacaggtcatgcagaccattccacctggtttattctggcgtttccagattactatccaccatccaatatatctgaagttcaatatttctttagccaaggactctctgctgggaatttatggcagaagaaacattccacctacacatactcag

SEQ ID NO: 822 - DNA, 24 bp, homo sapiens
ctcactacttagatgctgtccgag

SEQ ID NO: 823 - DNA, 217 bp, homo sapiens
atggtttaaccgactgtgtggatcctgactgttgtcaacaaagcaactgttatataagtcctctctgccagggctcaccagatcctcttgacctcattcagcaaagccaaactctcttctctcagcacacttcaagacttttttatgatcgaatcaaattcctcattggcaaggacagtactcatgtcattcctcctgaggtgtcatttgacagcag

SEQ ID NO: 824 - DNA, 120 bp, homo sapiens
gcgtgcctgtgtgattcgaggccaagtggtggccatagatggaactcctctagtgggagtgaatgtcagtttcttgcaccacagtgattatgggtttaccatcagccggcaagatggaag

SEQ ID NO: 825 - DNA, 262 bp, homo sapiens
ctttgacctcgtggccatcggtggcatctctgtcatcttaatcttcgaccgatcccctttcctgcctgagaagagaacactctggttgccttggaatcagtttattgtggtagagaaagtcaccatgcagagagttgtatcagacccgccatcctgcgatatctccaactttatcagcccaaaccctattgtgcttccttcaccgctcacatcatttggagggtcctgtccagagaggggaactattgttcctgagctgcag

SEQ ID NO: 826 - DNA, 268 bp, homo sapiens
gttgtacaggaggaaattcccattccctccagctttgtgaggctgagttacctgagcagccgcacccctgggtataaaaccctgctacggatccttctgacacattcaacgattcctgtaggcatgataaaagtacacctcacagtagctgtggaagggcgactcacacagaagtggtttcccgccgcaattaatcttgtctacacatttgcttggaacaagaccgacatctatggacagaaggtttggggcctggcagaggctttgg

SEQ ID NO: 827 - DNA, 144 bp, homo sapiens
tatctgtgggatatgaatatgaaacgtgccctgactttattctctgggagcaaaggacagtcgttttacaaggttttgagatggatgcttctaacctaggaggctggtctttgaataagcatcacattttgaatcctcaaagtg

SEQ ID NO: 828 - DNA, 250 bp, homo sapiens
gaatcatacataaagggaatggagaaaatatgttcatttcccagcagcccccagtcatatcaaccataatgggtaatggacaccaaaggagtgtagcctgcaccaactgcaatggcccagcccacaacaacaaactctttgctcctgtcgccttagcttctggccctgatggcagtgtgtatgttggcgacttcaattttgtaaggagaatatttccctcgggaaactccgttagtattttggaattaag

SEQ ID NO: 829 - DNA, 155 bp, homo sapiens
gcatcacagttgataggcatggatttatttactttgtggatgggactatgattcgcaaaattgatgagaatgctgtgatcacaactgtaatcggctcaaatggtctgacttccacacaaccactgagctgtgactcaggaatggacatcactcag

SEQ ID NO: 830 - DNA, 367 bp, homo sapiens
gtgcgattagagtggccaacagaccttgcagtaaatcctatggacaattcattgtatgtcttggataacaacattgtgctgcaaatttctgagaacaggcgtgttcggatcatcgcaggacgccccattcactgccaggtgccaggcatcgatcatttcctggtcagcaaggtagcaattcactccactctagagtcagcgagggccatcagtgtctcccacagcgggctgctcttcatagctgaaacagacgagaggaaagtaaaccgcattcagcaagtaaccaccaatggggagatctacatcatcgctggtgcccccactgactgtgactgcaaaattgatccaaactgtgactgtttttcag

SEQ ID NO: 831 - DNA, 511 bp, homo sapiens
gtgatggtggctatgccaaagatgcaaagatgaaagccccttcctccttagcagtgtcgcctgatggaaccctctatgtggcagacctcggaaatgttcgaattcgtaccatcagcaggaaccaagcccacctgaatgacatgaacatttatgagattgcttcacccgctgatcaggaactgtaccagttcactgtaaatggaacccacctacacaccctgaacttgataacaagggactatgtttataacttcacctacaattctgaaggtgacttgggcgcgattaccagcagcaatggcaattcagtgcacattcgccgtgatgcaggcggaatgccgctatggcttgtggtgcctggcggacaagtatactggctgactataagcagcaatggagtcctgaaaagagtgtcagcccaaggctataatctggccttaatgacctatccaggaaacacagggcttctggctaccaaaagtaacgaaaatggatggacaaccgtttatga

SEQ ID NO: 832 - DNA, 236 bp, homo sapiens
aaaatactcaaagtacctatcgggtgaatccagatggttccctgcgtgtcacttttgccagcgggatggagatcggcctcagctcagagccccacatcctggcaggggcagtcaaccctaccctgggcaaatgcaacatctcattgcccggagagcacaatgcaaacctcatcgagtggcggcagaggaaggagcaaaacaaaggcaatgtttcggcttttgaaaggaggctgagg

SEQ ID NO: 833 - DNA, 297 bp, homo sapiens
gcccacaacagaaacctactctccatagattttgatcatataacccgcacaggaaagatctatgatgaccatcgaaaattcacccttcgaattctttatgaccagactgggcgacccattctgtggtctcctgtaagcagatataatgaagtgaacatcacatattcaccttcgggattggtgacgtttattcaaagaggaacgtggaatgaaaaaatggaatatgaccagagtgggaaaattatttcaagaacttgggctgatgggaaaatttggagctatacctacttagaaaaa

SEQ ID NO: 834 - DNA, 388 bp, homo sapiens
tctgtgatgcttctcctacacagccagcggcgttacatctttgagtatgaccaaccagattgtctgctctcagtcaccatgcctagcatggtgcgtcacagcttacaaaccatgctttcagtgggctactaccggaacatctacaccccaccggacagtagcacttcttttatccaagactatagtcgagatggccgattgctacagaccctgcatctggggacagggcgcagagtcttatacaagtacaccaagcaagcaaggctttctgaggttctctatgataccactcaggtcacattaacatatgaagagtcttctggagtgattaagacaatacacctgatgcatgacggattcatctgcacaatcagatacaggcaaacag

SEQ ID NO: 835 - DNA, 1221 bp, homo sapiens
gacctcttattggacgccagattttcagattcagtgaagaaggccttgtgaatgcacggttcgactacagctacaacaatttccgagtcacaagcatgcaagctgtaatcaatgaaacccctttgcctatagatctttaccgatatgttgatgtctctggcagaacagagcagtttggaaaattcagtgtaattaattacgatttaaatcaggtcataactactacagtgatgaaacacaccaaaatcttcagtgccaatggacaagtcattgaagtccaatatgaaatcctaaaggcaattgcctactggatgaccattcaatatgataatgtgggccgcatggtaatatgtgacataagggtaggagtagatgccaatataacaaggtacttctatgaatacgatgctgatgggcaacttcagactgtttctgtaaatgacaaaacccagtggcgttatagttacgatctgaatggaaacatcaacctcttaagccatgggaagagtgctcgtcttactcctctccgatatgacctccgagaccgcatcaccagattaggagaaattcagtataaaatggatgaagatggctttctgaggcagaggggaaatgatatttttgaatataattctaatggcctgctgcagaaagcctacaataaggcttctggctggactgtgcagtattactatgatgggcttgggcgacgtgtcgcgagtaagtccagcctagggcagcaccttcagttcttttatgcagaccttaccaaccccataagagttactcatttgtacaaccacacaagctcggagattacatctctgtattatgatctccaaggtcaccttattgccatggagttaagcagtggtgaagaatattatgtagcctgtgataatacaggtaccccactagctgtgttcagcagccgaggtcaggtcataaaggagatactatacacaccttatggcgatatctatcatgacacttaccctgactttcaggtcattattggttttcatggaggactctatgatttccttactaaattagtgcacctggggcaaagggattatgatgttgttgctggcagatggacaacgcctaatcatcacatatggaaacagttgaacctccttcctaaaccattcaacctctactcctttgaaaataactacccagttggcaaaattcaagatgttgcaaagtataccacag

SEQ ID NO: 836 - DNA, 143 bp, homo sapiens
acatcagaagttggttggagctatttggtttccaattacacaatgtactacctggatttcccaaacctgaattagaaaatttagaattaacttacgagcttctacggcttcagacaaaaactcaagagtgggatcctggaaag

SEQ ID NO: 837 - DNA, 788 bp, homo sapiens
actatcctgggcattcagtgtgaactccagaaacagctcaggaatttcatttccttggaccaactacctatgactccccgatacaatgatggacggtgccttgaaggagggaagcaaccaaggtttgctgctgtcccttctgtttttgggaaaggtataaaatttgccatcaaggatggcatagtaacagctgatattataggagtagccaatgaagatagcaggcggcttgctgccattctcaataatgcccattacctggaaaacctacattttaccatagaggggagggacactcactacttcattaagcttgggtctctggaggaagacctggtgctcatcggtaacactggggggaggcggattctggagaatggtgtcaatgtcactgtgtcccagatgacttctgtgttgaatgggaggactagacggtttgcagatattcagctccagcatggagccctgtgcttcaacatccggtatgggacaactgtcgaagaggaaaagaatcacgtgttggagattgccagacagcgcgcagtggcccaggcctggactaaggaacaaagaaggctgcaagagggggaagaggggattagggcatggacagaaggggaaaagcagcagcttttgagcactgggcgggtacaaggttacgatgggtattttgttttgtctgttgagcagtatttagaactttctgacagtgccaataatattcactttatgagacagagcgaaataggcaggaggtaacaaaaatatctctgcctttgcgtcaccaa

SEQ ID NO: 838 - DNA, 4601 bp, homo sapiens
agactgcctgtttttaaaacataaaatggtttattgtattggttttctagatcagaactctgtatatgtaaatatggaggaaaaacatatccaactgcctttcaatgtgacggaagatggtattttaatattgtttgtttaaactctttaagaaatgacagagatttttagttcttgtgtggcagtattcaaaataacacaagtagaactcaaacagctaaaaacagttttcagaaagcaccactttcaatttgccgagccatgcatatgttccaatatccagaaagaacccaaggttctctatctctattgtgagaagcagtttcatccttaactgttggcagaacttacgggctatttgaataggtggtgcaatagtatctgaaacttgcctttcgaaagactgccagccctttgacgttttccagatctgttataggaaacttaaaaacaggtgtaaaatgtcttcagccaccatctcctagagtgaggacccaattgcccttccttcttgattattcctccttgcttgttaaagtaaatgccatattgttgtgctgtgttttggcgtgtggtggctgggttctgtctaccatgcttccctgtgggtgtggtaaccagactgtatagccgctatttgctcgtgtgtacatgataccaaagcagctggccagcgtgacctctctcacacgacctgttttgactcaattttttactaaaagttgttcagctgtattggtatcatgtaaacatagcttttattaacctgggtaggaatttctcatttatatataggatgtgttttggtcatagtttcacattagtgattcagtatctatacactgacccaatggttttgtgcacatgaacggtaatttacttaaaagtatgattctggtacaaaaacaaacaaaggctttagcaggcatacgtgtctgggatgccgatacatacattaactactactgcagaaattcataagagccaaaaccttaaaaaaatagacctggtacttaagtgaaagtactaaagggaagaccagaccaaacatcacagcagttgctgccacattgtttcagcccacttagatttatctttcaaatgtacaattctgtattgaacatctcccagccatcttcaggaaatcgaatcaagtaaatcctttccaaccgaaaacatttcaactaactatagagaggcagactcatttttactaaaataatttatacagttagttattttcgttctccgtacttacccatttatctttatttaatcgtctctactgcctaggaaaataactattttccaggacgggttatttgttctgcgatcatttaaaatttggagaaaggtcaggattagtgttaatatcagctgcagtttctcaatctctaggaatcctgcagtaaaacaagccccttggtgagctggaagatttgtgcccagtgacaaagagatagtttgtaaaatgctgtgtaattgtaagttaccacaaatgaaaatacatgacagcacaatgtggcccgtagaaaattcccctgagccagcttctgcactttcatcaccgaatctgaacatttgctatgtctgaaggcaaatttatgatggaatgttagtttggattctttccagatgctacctaaatgcagtgtggggtcattgccttgctttgcgatgacagtttctttgaaaatatgcaaagtcataagctcatgttaaggtttttcaagagtctgcctcctactacacaaaggaaagcaagggaaaggaaatgaccctggcaaacagtagggaagggtgtattcaaacatttcattttcaaaaccttcgggttagaataccacttacacatgtattctgagagacagaattcatgaggaactcatctctctttataactggaaacacaccagcttgatatattgctaatccatactaaaatcatattattgggttttttctgaatcaggcctgtattaatggtacagtatttattcagaatggaattctaaaattactaacaaacttgttgaaaatttgaatacctccacaccaacctaaaaatggaccttaagttcctagaacctctgatgttcttttaaattaatggaaaaataatttgtgaactgtatatagagagtgcattcataaatgtgattatgtattttatcacaaatccaaaatgtcaatattagagtctattttgcttatattttaagcaattatacgtttttgcaattcattgatgatgtatcattttcaaactgctttaaatatccattagaaacaaatatttgaagcttttacttaatagtgattaccttgaactgtgcatttctagtttgtaatacgtatttggttggttcgtgcctttagtttgttaaagttacatttgtattatattcaggaaatgcactttttattacttacagctgtggttttaatactgccttgaactattattattctttttacaactcctaaagcttgagggaggaaagaaaaaaaaaacaaaactactaatcagtagtaaatcgaagagaaacattttggcatttcttaagaagaagatggagatattgagtatatcacttcctattcagctgaatagaaagaatgccttcattgacttgcagttctgcagtttaaattattgaaagaacaattcgtttgcatttcctgatgaaagtaaaagcatttttcagagaaacatatgaatttctcatacccagcagacagatggctgacactgcacagccacacaccattcgagtaagttaaagtgagagcatagtagttggactctcctatgaagaacattctgggctggaggcagggaatactccatggttgtttctttttcctacttaagcccattttgtttgtgcttttctgttttgttttgttttcactcttgcactacagtctagagatccaaatgaactgaaaagttcaaagtttaacacatttaaatatgtttacttttagttgtcattctaatcgttattgattagaagcatgactcctgaaggaaagggaaataaatctcaattcatactaacttgcaacaaaacacttttaccatataaataagtatatgatttatttttaacccaaaaaatgtataaaataagtgtgtcctttactgtcaatttatctagaagatctataatatatagactacatatatataatatatacaacatagccaaatgtatgaaaacttgacaatgtataatttggaattcacatgctacctatgtagacaggtatgaaattaagttataattttcatgagacattttcatcactgttgacacagtttcaagcattccatcatgttattttgactctttttcttttttttttctttaaaaatatatttttaactagaccaggccccactataatatcacttaagagagtcagggcaaagtttttgcatttatgaagatgtgttcatgtaagggtgattgtaatggagttcattggtaatagaagcaaaagtacagtaacgaagtattgaaaagaaaattttggagacattggagcatattatatatagcttgtggaaagacataaggctacagatggaatggaacattcctgttttcttgaagaaattcacatacacatagctgacctgactagtacttcagctcttccacagccttctataaaggttctttcttctgcaaagaaaacaaaacaaaacaaaacaaaacaaaaaaaacaaaaaaagcgcaaaaaacaaaaaaacaaaaaaaagcaaagtaaaatttaaaaatacagaaaacaaacaacaaaaaagaattcaaccataaatagtgactattattttcagtgtgtccttcatgtgaaagctattaaggaccaaatatactactgttcataagaagaaattactttctaaacagtaactgaaaatacttagagttaaacttgctgtggattttgtcttggcagttgtcatcttacattatttgtcaaaggaaatgtgtttggcagttaaaaatctttccttagatttagtggtggactttaacctcttaaataaatgttagtatatcagattgtgtccttgaaaaatattttacttgtatgaatcatgacaacgtctaaatctttactattcttctggcaaaagcatcagtaagaaagaaggcgaaaaagagaagtatagcctttatgtcagaaaaacattctttttagctgcttactttctcatgaaaagtaaagatgtttacagtgtatgccaagttttcagtttctgtataacaacaggtagaggttctaatcatattgaaaattgtgttataatggtctgagccatgttgctaggaaacaataggttccaattttgtattcctgctctcctgtgctgaaaagtgactggatactgtacaggttcatgttctctggctgcagttaaatggtcttttgcattttgctctggctttcaggccagaagcatgcatttttctacaagagcatcacaacaacatgctgtaaatatttaaagttaaacattatgtgttgatatttgaaagaaaagtactttgaatatttcatttttaaaaaataaaattgccaatgaa

SEQ ID NO: 839 - DNA, 111 bp, homo sapiens
ccgaagggagccgatcggaaacagaagactgaccgggagaagatggagaaaagaactgcccaagagaaggagaaataccagccgtcctatgaaaccaccatcctcacagag

SEQ ID NO: 840 - DNA, 694 bp, homo sapiens
tggtctgaagagcccagaggaggagtttggccaggtggactgtggcagatcaataaagaaaggcttcttcaggacagcactgccagagatgcctgggtgtgccacagaccttcctacttggcctgtaatcacctgtgcagccttttgtgggccttcaaaactctgtcaagaactccgtctgcttggggttattcagtgtgacctagagaagaaatcagcggaccacgatttcaagacttgttaaaaaagaactgcaaagagacggactcctgttcacctaggtgaggtgtgtgcagcagttggtgtctgagtccacatgtgtgcagttgtcttctgccagccatggattccaggctatatatttctttttaatgggccacctccccacaacagaattctgcccaacacaggagatttctatagttattgttttctgtcatttgcctactggggaagaaagtgaaggaggggaaactgtttaatatcacatgaagaccctagctttaagagaagctgtatcctctaaccacgagaccctcaaccagcccaacatcttccatggacacatgacattgaagaccatcccaagctatcgccacccttggagatgatgtcttatttattagatggataatggttttatttttaatctcttaagtcaatgtaaaaagtataaaaccccttcagacttcta

SEQ ID NO: 841 - DNA, 191 bp, homo sapiens
gagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaag

SEQ ID NO: 842 - DNA, 693 bp, homo sapiens
atcccttcacatgatagaagacagactctttgatgaggtcggcggagcagttcactagccaatgatgagagcagaaaggcctagacctgcagccagaagtgaaggcggctcagttctccgggatgcttctctacctcctgagcaccaattcctggattccagtcactggctcacctttagaatgtctgttgctattcactgctcccctcgctcctcttaacagcttggggaggtgaccagtggttcaggagggactagacaattacctgtccagtgtggtatggtaggaagagtgtaggtgttggcacgtgaccaaaattcacatccctcctcatggcagtcattcagtatgtgtacttgtacaagttatttaacccattggagcctaaattccctcatctataaaatggggataatattatctacctcacaagcttatgaaaactaaacatgatgaatcaaaagcacttggcatgtgagggctattaaaatagcctgattttttttttctccccctctccccaatgtatttgctctggcccttgctttttaccctccagagctaagaggtagcagagtctcttgggatgagtgattcaccctcttacttggcgaccactgatgagatcaacaacaggtgaactataaacctattatttattgcagaactaataaaaaatccaaagccttgta

SEQ ID NO: 843 - DNA, 958 bp, homo sapiens
gatttttagactctgaggagcagttggagctaatccacattatggaaatggaaaccaccgaacctgagccagactgtgtagtgcagcctccctctcctcctgatgacttttcatgccaaatgagactctctgagaagatcactccattgaagacttgttttaagaaaaaggatcagaaaagattgggaactggaaccctgaggtctttgaggccaatattaaacactcttctagaatctggctcacttgatggggtttttagatctaggaaccagagtacagatgagaacagcttacatgaacctatgatgaagaaagccatggaaatcaattcatcatgcccaccagcagaaaataatatgtctgttctgattcctgataggacaaatgttggggaccagataccggaagcccatccttccactgaagctccagaacgagtggttccaatccaagatcacagctttccatcagaaaccctcagtgggacggtggcagattccacaccagctcacttccagactgatcttttgcacccagtttcaagtgatgttcctactagtcctgactgcttagacaaagtcatagattatgttccaggcattttccaagaaaacagttttacaatccaatacattctggacaccagtgataagctgagtactgagctctttcaggacaaaagtgaagaggcttcccttgacctcgtgtttgagctggtgaaccagttgcagtaccacactcaccaagagaacggaattgaaatttgcatggactttctgcaaggcacttgtatttatggcagggattgtttgaagcaccacactgtcttgccatatcattggcagatcaaaaggacaactactcaaaagtggcagagtgtattcaatgattctcaggagcacttggaaagattttactgtaacccagaaaatgatagaatgagaatgaagtatgg

SEQ ID NO: 844 - DNA, 169 bp, homo sapiens
aggacaagaattttgggcagatttgaatgccatgaacgtgtatgaaacaactgaatttgaccaactacgaaggctgtccacaccaccctctagcaatgtcaactctatttaccacacagtctggaaattcttctgtagggaccactttggatggagagagtatcccgag

SEQ ID NO: 845 - DNA, 437 bp, homo sapiens
gaaaaaggaatatatgaacaggaaaatgtttggccgtgacaggataataaatgagagacatttatttcatggaacatcccaggatgtggtagatggaatctgcaaacacaactttgaccctcgagtctgtggaaagcatgctacaatgtttggacaaggcagttattttgcaaagaaggcaagctactctcataacttttctaagaagtcctccaaaggagtccacttcatgtttctggccaaagtgctgacgggcagatacacaatgggcagtcatggcatgagaaggcccccgccagtcaatcctggcagtgtcaccagtgacctttatgactcttgtgtggataatttctttgagcctcagatttttgtcatttttaatgatgaccagagttacccttattttgttatccaatatgaagaagtcagtaacactg

SEQ ID NO: 846 - DNA, 41 bp, homo sapiens
aaaacgcacaagccagaccctggcacgccccagcacacgag

SEQ ID NO: 847 - DNA, 114 bp, homo sapiens
gacccttcttctccgcgagcaagccctgcccactcgccccgggaaaatggaatcgacaaaaatcgcctgctaaagaaggatgcttctagcagtccagcttccacggcctcctcg

SEQ ID NO: 848 - DNA, 623 bp, homo sapiens
tccaaagagtcctcgtcagtgcttagctgtgacatctctgtggatgataagtacatagtcactggctcgggggacaagaaggctacagtctatgaagtcatctactgaaaacattatgtggtttaacgtttatagttgaattgggccaaaatgtttcgaatttatagaaatagaaaagttgtaactttaaaagagaaaaaaaattacaaacacctgtttccaaaccttgacagaaaactactttgagtctacaaagaggaggcgacaagtccatcagcagaaagtcacctgtctacatagaccaaatggagcaccaaggccaagcggacagaggggccatgggttgtaggattgaggaacggaatctgccgactcacatgacagcccattctttctttctgggtgatctggggatcacgccttgcccaagtgtgagattacctttctgttccttgcagttcacctcactttccgtcctttgtagagcagtggtgtctccaatgaacttgtttcctggttttgcatcttgtgaaatgtttttttgtatttttgttgaaggttaaacatttgtataaattgtaaatatatttggtttattacagtaaaggctttagtaccaataa

SEQ ID NO: 849 - DNA, 135 bp, homo sapiens
cctggaagcaagatggtgtcctctgccaaagccgcagtgcccaccgtgagtgaccaggccgcagccatgcagctgagccagtgtgccaagaacctggccaccagcttggcggagctgcgtaccgcctcgcagaag

SEQ ID NO: 850 - DNA, 85 bp, homo sapiens
tgatggagaaccttgtgacctgtctttgaatataacctggtatctgaaaagcgctgattgttacaatgaaatctataacttcaag

SEQ ID NO: 851 - DNA, 34 bp, homo sapiens
cactatcttcctgaagtttgatggagaaccttgt

SEQ ID NO: 852 - DNA, 108 bp, homo sapiens
gaagtagagttgtatttggaaaaacttaaggaaaaaagaggcttgtctgggaaatatcaaacatcatcaaaattgttccagaactgcagtgaactctttaaaacacag

SEQ ID NO: 853 - DNA, 49 bp, homo sapiens
accttttctggagattttatgcatcgactgcctcttttaggagaaaaac

SEQ ID NO: 854 - DNA, 34 bp, homo sapiens
ttggattggtgctgtcatcttcctgggaatgctt

SEQ ID NO: 855 - DNA, 34 bp, homo sapiens
ggatatggcatcgtcaagccacgccttggagtca

SEQ ID NO: 856 - DNA, 34 bp, homo sapiens
gccttgtgctggtggatatttattagcctgactc

SEQ ID NO: 857 - DNA, 34 bp, homo sapiens
tagtgacatgtcagtcggactggcgggagctgtg

SEQ ID NO: 858 - DNA, 70 bp, homo sapiens
gccttttcaccattgtctgaggaagaggaggaggatgaacaaaaggagcctatgctgaaagaaagctttg

SEQ ID NO: 859 - DNA, 34 bp, homo sapiens
tctgcaaacaaccagaggtttgccttttcaccat

SEQ ID NO: 860 - DNA, 595 bp, homo sapiens
gaacgaatgatcacacactttgaaaggtccaaaatggagtaaggaatgggaagatttgcagttaaagatggctaccatcagggaagagatcagcatctgtgtcagtcttctgtacggctccatgggattaaaggaagcaatgacatcctgatctgttccttgatctttgggcattggagttggcgagaggtgtcagaacaaagagaacatcttactgaaaacaagttcataagatgagaaaaatctacgagcttcttatttacaacactgctgccccctttcctcccagactctgacatggatgttcatgcaacttaagtgtgttgttcctgaactttctgtaatgtttcattttttaaatctgacaaactaaaaagtttaacgtcttctaaaagattgtcatcaacaccataatatgtaatctccaggagcaactgcctgtaatttttatttatttagggagttacataggtgatgggggaaattgttaactacctttcattttcctgggaagtcaaggttacatcttgcagaggttgttttgagaaaaaagggcccttctgagttaaggagccatagttctatcaatgatcaa

SEQ ID NO: 861 - DNA, 86 bp, homo sapiens
ttgatggagaaccttgtgacctgtctttgaatataacctggtatctgaaaagcgctgattgttacaatgaaatctataacttcaag

SEQ ID NO: 862 - DNA, 114 bp, homo sapiens
gcagaagaagtagagttgtatttggaaaaacttaaggaaaaaagaggcttgtctgggaaatatcaaacatcatcaaaattgttccagaactgcagtgaactctttaaaacacag

SEQ ID NO: 863 - DNA, 54 bp, homo sapiens
accttttctggagattttatgcatcgactgcctcttttaggagaaaaacaggag

SEQ ID NO: 864 - DNA, 62 bp, homo sapiens
tgactgttgaagtgaagggtccctatgaatacctcacacttgaagactatcccttgatgatt

SEQ ID NO: 865 - DNA, 127 bp, homo sapiens
gcccattgtggacctcattggtgccatggagacccagtctgagccctctgagttagaactggacgatgtcgttatcaccaacccccacattgaggccattctggagaatgaagactggatcgaagat

SEQ ID NO: 866 - DNA, 34 bp, homo sapiens
gggtctcatgtcccactgcattgccatcttgaag

SEQ ID NO: 867 - DNA, 116 bp, homo sapiens
atttgtcacactctgacagagaagcttgttgccatgacaatgggctctggggccaagatgaagacttcagccagtgtcagcgacatcattgtggtggccaagcggatcagccccag

SEQ ID NO: 868 - DNA, 135 bp, homo sapiens
gcgctgcagcacctcctgcccccttccggctgctttggcccatccttgggggcgctctgagcctgaccttcgtgctggggctgctttctggctttttggtctggagacgatgccgcaggagagagaagttcacca

SEQ ID NO: 869 - DNA, 91 bp, homo sapiens
gggctgagtcacctgcaccagcataaagtgattcatcgagatattaaagggcaaaatgtcttgctgactgaaaatgcagaagttaaactag

SEQ ID NO: 870 - DNA, 131 bp, homo sapiens
tggactttggagtcagtgctcagcttgatcgaacagtgggcaggaggaatactttcattggaactccctactggatggcaccagaagttattgcctgtgatgaaaacccagatgccacatatgatttcaag

SEQ ID NO: 871 - DNA, 34 bp, homo sapiens
ccacatatgatttcaagagtgacttgtggtcttt

SEQ ID NO: 872 - DNA, 79 bp, homo sapiens
ctctctgtgacatgcaccccatgagagctctcttcctcatcccccggaacccagcgcctcggctgaagtctaagaagtg

SEQ ID NO: 873 - DNA, 205 bp, homo sapiens
ctccatcctgaatctgccaggggagtcgacgctgcggagggactttctgaggctgcagctggccaacaaggagcgttctgaggccctacggaggcagcagctggagcagcagcagcgggagaatgaggagcacaagcggcagctgctggccgagcgtcagaagcgcatcgaggagcagaaagagcagaggcggcggctggaggag

SEQ ID NO: 874 - DNA, 189 bp, homo sapiens
gaatataagcgcaaacaattggaagaacagagacaagcagaaagactgcagaggcagctaaagcaagaaagagactacttagtttcccttcagcatcagcggcaggagcagaggcctgtggagaagaagccactgtaccattacaaagaaggaatgagtcctagtgagaagccagcatgggccaaggag

SEQ ID NO: 875 - DNA, 165 bp, homo sapiens
gtagaagaacggtcaaggctcaaccggcaaagttcccctgccatgcctcacaaggttgccaacaggatatctgaccccaacctgcccccaaggtcggagtccttcagcattagtggagttcagcctgctcgaacaccccccatgctcagaccagtcgatccccag

SEQ ID NO: 876 - DNA, 98 bp, homo sapiens
ccaacagtaagtcagaaggatcacctgtgctcccccatgagcctgcgaaggtgaaaccagaagaatccagggacattacccggcccagtcgaccagct

SEQ ID NO: 877 - DNA, 165 bp, homo sapiens
acgtctggagagaagaagcgatctggccacagtgacagcaatggctttgctggccacatcaacctccctgacctggtgcagcagagccattctccagctggaaccccgactgagggactggggcgcgtctcaacccattcccaggagatggactctgggactgaa

SEQ ID NO: 878 - DNA, 171 bp, homo sapiens
ctctgtttactagcgaacttcttaggcaagaacaggccaaactcaatgaagcaagaaagatttcggtggtaaatgtaaacccaaccaacattcggcctcatagcgacacaccagaaatcagaaaatacaagaaacgattcaactcagaaatactttgtgcagctctgtggg

SEQ ID NO: 879 - DNA, 140 bp, homo sapiens
cctacattcattccaatcagataatgggctggggcgagaaagctattgagatccggtcagtggaaacaggacatttggatggagtatttatgcataagcgagctcaaaggttaaagtttctatgtgaaagaaatgataag

SEQ ID NO: 880 - DNA, 140 bp, homo sapiens
gcctcggatgccctcaaggcctgaattgctggtgaagccagccctgccggatggttccttggtcattcaggcttcagaggggccgatgaaaatacagacactgcccaacatgagatctggggctgcttcacaaactaaag

SEQ ID NO: 881 - DNA, 463 bp, homo sapiens
gttgcaaataaaaatgtggctaaagtaccacctcctgttcctacaaaaccaaaacagattaatttgccttattttggacaaactaatcagccaccttcagacattaagccagacggaagttctcagcagttgtcaacagttgttccgtccatgggaactaaaccaaaaccagcagggcagcagccgagagtgctgctatctcccagcataccttcggttggccaagaccagaccctttctccaggttctaagcaagaaagtccacctgctgctgccgtccggccctttactccccagccttccaaagacaccttacttccacccttcagaaaaccccagaccgtggcagcaagttcaatatattccatgtatacgcaacagcaggcgccaggaaaaaacttccagcaggctgtgcagagcgcgttgaccaagactcataccagagggccacacttttcaagtg

SEQ ID NO: 882 - DNA, 38 bp, homo sapiens
tactccattcgccattccataagtatgcctgctatgag

SEQ ID NO: 883 - DNA, 39 bp, homo sapiens
ttactccattcgccattccataagtatgcctgctatgag

SEQ ID NO: 884 - DNA, 235 bp, homo sapiens
gttaccattaagaatatcgaaagggagctcatttgcccagcatgcaaggagctgtttacccacccattgattctcccttgccaacatagtatctgtcataaatgtgtaaaagaactcctgctgactctcgatgattcattcaacgatgtgggatcagacaactccaatcaaagcagtcctcgacttcggctcccctcccctagtatggataaaattgaccgaattaacagaccag

SEQ ID NO: 885 - DNA, 34 bp, homo sapiens
gccttggcagagctgagcaggcattttggagatc

SEQ ID NO: 886 - DNA, 146 bp, homo sapiens
ggtgtttttggctgcaattgcatgaaatcccaatggtgtagaccagtggcgatggatctaggagtttaccaactgagacatttttcaatttctttcttgtcatccttgctggggactgaaaacgcttctgtgagacttgataatag

SEQ ID NO: 887 - DNA, 123 bp, homo sapiens
cgcccacggtctcgcttcctgaactccgttcactcctagcctccggacgggcccggctcttcgacgtgcgctctcgcgaggaggcggcagctgggaccatcccaggggcgctcaacatcccgg

SEQ ID NO: 888 - DNA, 138 bp, homo sapiens
cgggttttcagaccacccaactactctggtaccattgctttggccctgttagtgtcgcttgttggaggtttgctttatttgagaaggaacaacttggagttcatctataacaagactggttgggccatggtgtctctg

SEQ ID NO: 889 - DNA, 77 bp, homo sapiens
ctatgacttctggccagatgtggaaccatatccgtggacctccatatgctcataagaacccacacaatggacaagtg

SEQ ID NO: 890 - DNA, 30 bp, homo sapiens
ttcgttccaagtaccacggctatccttata

SEQ ID NO: 891 - DNA, 65 bp, homo sapiens
tgatctggactttgagtgagaagatgtgatttggaccatggcacttaaaaactctataacctcag

SEQ ID NO: 892 - DNA, 141 bp, homo sapiens
attcgggttttcagaccacccaactactctggtaccattgctttggccctgttagtgtcgcttgttggaggtttgctttatttgagaaggaacaacttggagttcatctataacaagactggttgggccatggtgtctctg

SEQ ID NO: 893 - DNA, 90 bp, homo sapiens
tgtatagtctttgctatgacttctggccagatgtggaaccatatccgtggacctccatatgctcataagaacccacacaatggacaagtg

SEQ ID NO: 894 - DNA, 284 bp, homo sapiens
ctttttaattaaatgaagccaagtgggatttgcataaagtgaatgtttaccatgaagataaactgttcctgactttatactattttgaattcattcatttcattgtgatcagctagcttattcttgtgtactttttttaaactgtgggttttcctagtaaatttaatttacagaaatcaatggtagcatttagtaatctacaaaggaaatatcaaagtgtttttcaagcctgttatattcagtgtgtgccacaggattgaaataaatgacaatgtaattatgaa

SEQ ID NO: 895 - DNA, 34 bp, homo sapiens
gctcattaaaggattcggttgccgaggagcccaa

SEQ ID NO: 896 - DNA, 44 bp, homo sapiens
gccagttcatgggttaatttttcttttcaagtggcagccaggag

SEQ ID NO: 897 - DNA, 126 bp, homo sapiens
gtaattaataatgcttgtgctactcaagccatagtgagtgtgttactgaactgtacccaccaggatgtccatttaggcgagacattatcagagtttaaagaattttcacaaagttttgatgcagct

SEQ ID NO: 898 - DNA, 62 bp, homo sapiens
atgaaaggcttggcactgagcaattcagatgtgattcgacaagtacacaacagtttcgccag

SEQ ID NO: 899 - DNA, 64 bp, homo sapiens
gtgcatgcaatcaagatgattggatcagtgcagtaaggcctgtcatagaaaaaaggatacaaaa

SEQ ID NO: 900 - DNA, 103 bp, homo sapiens
gtacagtgaaggtgaaattcgatttaatttaatggccattgtgtctgacagaaaaatgatatatgagcagaagatagcagagttacaaagacaacttgcagag

SEQ ID NO: 901 - DNA, 34 bp, homo sapiens
gccgccggggattccaggttgccgaggagcccaa

SEQ ID NO: 902 - DNA, 106 bp, homo sapiens
gccagttcatgggttaatttttcttttcaagtggcagccaggagaagaaccagcaggctctgtggttcaggactcccgacttgacacgatattttttgctaagcag

SEQ ID NO: 903 - DNA, 82 bp, homo sapiens
gagaatcctgattattctgaagaaatccttaagggcgagttggcatggatcatctacaaaaattctgtaagcataattaaag

SEQ ID NO: 904 - DNA, 34 bp, homo sapiens
gataaaatgccaatcaggttttcgaaaggcctta

SEQ ID NO: 905 - DNA, 34 bp, homo sapiens
gataaaatgccaatcagcaccattcaccgccttg

SEQ ID NO: 906 - DNA, 216 bp, homo sapiens
ggccaggtcatgaacattttcgtgatactccccgccctgatcatccccctcacgacggtcattccccagccagcagagaacgctcctcttctctccaaggcatggacatggcatccctacctccccgaaagcgcccctggcatgatggcccaggcacttctgagcacagagagatggaggccccaggaggcccttctgaagaccgaggaggcaaag

SEQ ID NO: 907 - DNA, 239 bp, homo sapiens
ggaggaagatggcgggcgggctgctctgaagagacctcggcggcggcggaggaggagagaagcgcagcgccgcgccgcgccggggcccatgtggggaggagtcggagtcgctgttgccgccgccgcctgtagctgctggacccgagtgggagtgagggggaaacggcaggatgaagttcgccgagcacctctccgcgcacatcactcccgagtggaggaagcaatacatccagtatgag

SEQ ID NO: 908 - DNA, 52 bp, homo sapiens
gctttcaaggatatgctgtattcagctcaggaccaggcaccttctgtggaag

SEQ ID NO: 909 - DNA, 224 bp, homo sapiens
agaagctcgcagaggctcagcgcaggtttgctacacttcagaatgagcttcagtcatcactggatgcacagaaagaaagcactggtgttactacgctgcgacaacgcagaaagccagtcttccacttgtcccatgaggaacgtgtccaacatagaaatattaaagaccttaaactggccttcagtgagttctacctcagtctaatcctgctgcagaactatcag

SEQ ID NO: 910 - DNA, 172 bp, homo sapiens
tttcgagtatttacagcccccttccataaggtaggctttgctgatttctggctggcggatcagctgaacagcctgtcagtgatactgatggacctggaatatatgatctgcttctacagtttggagctcaaatgggatgaaagtaagggcctgttgccaaataattcagaag

SEQ ID NO: 911 - DNA, 195 bp, homo sapiens
aatcaggaatttgccacaaatatacatatggtgtgcgggccattgttcagtgcattcctgcttggcttcgcttcatccagtgcctgcgccgatatcgagacacaaaaagggcctttcctcatttagttaatgctggcaaatactccacaactttcttcatggtgacgtttgcagccctttacagcactcacaaag

SEQ ID NO: 912 - DNA, 222 bp, homo sapiens
gcgatttgtgtggaacttcttccgcctggagaatgaacatctgaataactgtggtgaattccgtgctgtgcgggacatctctgtggcccccctgaacgcagatgatcagactctcctagaacagatgatggaccaggatgatggggtacgaaaccgccagaagaatcggtcatggaagtacaaccagagcatatccctgcgccggcctcgcctcgcttctca

SEQ ID NO: 913 - DNA, 459 bp, homo sapiens
gagttgatgcaggacggcgcgtccctgagcgacagcaccgaggacgaggaggagggggcgagcctgggcgacggcagcggggcggaaggcggcagctgcagcagcagcaggcggtcgggcggcgatggcggggacgaagtggagggcagcggtgtgggagctggcgaaggagagactgtccagcacttcccgctcgcgcggcccaagtctctaatgcagaagctccaatgctccttccagacctcctggctcaaggactttccctggctgcgctattccaaggatactggtcttatgtcttgcggctggtgccaaaagacccctgcagatgggggaagcgtggaccttcccccagtggggcatgatgagctttcgcgagggacccgcaactacaagaaaaccctcctcctgaggcaccacgtctctaccgagcacaaactccacgaagccaacgcccag

SEQ ID NO: 914 - DNA, 2605 bp, homo sapiens
ctttggctgaagtccaaatgacattcctataatctgcttatattctcctcctagagtttggaccatgcttcacctattgactacgatctaccttcagcataaatcaaccttcctggaaccacagctctgccacccttccatagagctatatgttttgtttccatctaaagactcatgctttcccaacaaacagaacctcctggctatttacaaaccagattgaatgggcatttgaatactgcttggaactgttggtccctttttctttgcaggctagcccatcaggcaggtgtatgcttttagccaagcccaccaattccaacttattcctctcttctccaacaaccctacttgaggttccagcatgtcttatagccaagtcaatatgctctaggataaggatttctgaaaggtatttagtgagagttggggatggtgtgtcataattactgtatctgtagcatgtccttgctgccttggacaagggacctggccaaataaatctccactgaagactgacagccagaccctgcagtagtttctaactctagaagcctcaatgtagtcaccatctaggattactactcattgctattgagctctacctttatggaccacctctgaagtccttcatctaattgaggatcttaaaaaacaatgaatggcaaaaaacttcctacactatctcatctgaaaaatcctggggcacatacattaaaaccatttgtgcacaaaactcaggaagtcgggattttattgcttatgttttactcttggtacacaccagctttgtgtagattcctgatgcttctgaacaaaagtagtgttatgctagaaaggcaaaactccctaaaatgttaaaactgttcagcaatgatcactgctggtaattaccagccctctgttccctgcttcttgtttctgatgtatgtccccaaaatggatatcctggagaaatatcagtaatcacatattccctagcaaccataccattggccttttccatgaagaagaggcaacattgagtggcggacaacactttttggcatctctccaattcagctgttagcatctcctagtgtcttcttcacagctaacagctttggctacttgctcatgacactccgtctctgatcacagagcaaatccactgtctccattggtttccagtactagtactcaattctactcaatcccacctgattccaggagacataatggagaagtgcacgaaatcatcatctaccatgcaggtagagccttcctttcttcaggcagagaatctgattctgcggcttcaaatgcagcatccaaccacggagaacactgctaaaaggggccaagtcatgcctgccctggccaccacagtgatgcctgtaccatactcacttgagcatctcacccagtttcatggtgaccctgccaattgctcagagttcctcactcaggtgactacctacttgacagctctccaaatctctaatcctgcaaatgatgcccagatcaaactcttttttgattacctatctcagcagttagaaagttgtgggatcatatctgggcctgacaagagtaccttactgaagcaatatgagaatcttattcttgagttccagcagtcatttggtaaacccacaaaacaggaaatcaatcctctgatgaatgctaagtttgacaaaggagacaactcctctcagcaggaccctgctactttccacctcctcgctcaaaatctgatctgtaatgaaaccaatcagagtggtcagttcgaaaaggcactagctgatcccaaccaggatgaagagagtgtcactgatatgatggacaatcttccagacctgatcactcagtgcattcagttggacaagaaacatagtgacaggccagagctcctacagtcagagacccagctcccattgttggcttccttgatccaacaccaagccctctttagccccacagatccaccacccaagaaagggcctatacagctgcgagaaggccagctgcctctcaccccagccaaacgagcccgccagcaagaaactcagttgtgcctctactgcagccaatctggtcacttcacaagagattgccttgccaaacgttctcgagctccggcaacgacaaataacacagctcaccagtaagaggagaccaggaaagtcactttttaaacttacatcccagccttactgatttataacctgcattaacttttaaaatacagatggggaactgtgacaccactttataggcagttatgaactgacacgctttgggggaatttaaacaatcagatcttgttcattggcaaattacccctcacgaaacctaacccgagaatgatgaaaacaattcacaggcactttctgcagttgggattaattctccaagagcaccaaaaacctgcaggcaaaactttgcccagtctgagccaatgaaaataaccgtggaagcacattgggaaccttatcctgaccaatgggtgacatctgggttgagtttgtgtcccaaatcctgaatcttgtgtgcctagaaatgagcccaagacttttaccttcacctttattggtctgtatcctagtttcctgttggcttagga

SEQ ID NO: 915 - DNA, 138 bp, homo sapiens
gaggtgtgtgagtcagcagaagacactgtcttggacttggtagactattgccaccgcaaactgaccctgctggtggcccagagtggctgtggtggcccccctgagggggagggatcccaggacagcaaccccatgcag

SEQ ID NO: 916 - DNA, 127 bp, homo sapiens
gtaactgtgacatttgatgatgtggccttatatttttcggaacaagagtgggagatcctggagaagtggcagaagcaaatgtataagcaagagatgaagaccaattacgagacccttgattccctgg

SEQ ID NO: 917 - DNA, 127 bp, homo sapiens
gactccatctcccttgaggatgtggctgtgaacttcaccctggaggagtgggctttgctggatcctggccagaggaatatctacagagatgtgatgcgggcaaccttcaagaacctggcctgtatag

SEQ ID NO: 918 - DNA, 101 bp, homo sapiens
gtgcagcagaaggaaagtggcttaaaatataaatcttttctggacttcgcgggtcttgatttgcagatcttctggaatttttacagtaaattaaagcaaaa

SEQ ID NO: 919 - DNA, 24 bp, Artificial
  probe
caccgctgagctgccgagaggaag

SEQ ID NO: 920 - DNA, 24 bp, Artificial
  probe
gcagttctcggtgaggaggaagag

SEQ ID NO: 921 - DNA, 24 bp, Artificial
  probe
tccaacttctgccaaggctctgaa

SEQ ID NO: 922 - DNA, 24 bp, Artificial
  probe
gtcgaggcctgcacttgatgtcaa

SEQ ID NO: 923 - DNA, 24 bp, Artificial
  probe
ggcctactccaaccttgcggtgaa

SEQ ID NO: 924 - DNA, 24 bp, Artificial
  probe
gtgaaagatcgcaaagcagtggcc

SEQ ID NO: 925 - DNA, 24 bp, Artificial
  probe
cctcgggatctccaataggctctc

SEQ ID NO: 926 - DNA, 24 bp, Artificial
  probe
ctcgggatctccaataggctctcc

SEQ ID NO: 927 - DNA, 24 bp, Artificial
  probe
cacttgctggctagtatgcacctg

SEQ ID NO: 928 - DNA, 24 bp, Artificial
  probe
tcactctctccttctgctggtgcc

SEQ ID NO: 929 - DNA, 24 bp, Artificial
  probe
atgaggaagtgctgatgtcgctgg

SEQ ID NO: 930 - DNA, 24 bp, Artificial
  probe
actgcggactttccatctagctgc

SEQ ID NO: 931 - DNA, 24 bp, Artificial
  probe
gcaaaccctaccagcctctaccaa

SEQ ID NO: 932 - DNA, 24 bp, Artificial
  probe
gggagaccctgcacgagtataacc

SEQ ID NO: 933 - DNA, 24 bp, Artificial
  probe
cctgtttgttgtgacatggaggct

SEQ ID NO: 934 - DNA, 24 bp, Artificial
  probe
ttgtgacatggaggctcaagctgg

SEQ ID NO: 935 - DNA, 24 bp, Artificial
  probe
gcccacacaaccagttcaccttca

SEQ ID NO: 936 - DNA, 24 bp, Artificial
  probe
agcacgcaggattcagtccatctg

SEQ ID NO: 937 - DNA, 24 bp, Artificial
  probe
tggttcctctgcgatcttcagtgc

SEQ ID NO: 938 - DNA, 24 bp, Artificial
  probe
gcgccactgcaatcacagtgactt

SEQ ID NO: 939 - DNA, 24 bp, Artificial
  probe
ctcactacttagatgctgtccgag

SEQ ID NO: 940 - DNA, 24 bp, Artificial
  probe
ctcactacttagatgctgtccgag

SEQ ID NO: 941 - DNA, 24 bp, Artificial
  probe
ggctcaccagatcctcttgacctc

SEQ ID NO: 942 - DNA, 24 bp, Artificial
  probe
gccaaactctcttctctcagcaca

SEQ ID NO: 943 - DNA, 24 bp, Artificial
  probe
gagaacactctggttgccttggaa

SEQ ID NO: 944 - DNA, 24 bp, Artificial
  probe
tgcttccttcaccgctcacatcat

SEQ ID NO: 945 - DNA, 24 bp, Artificial
  probe
cctgctacggatccttctgacaca

SEQ ID NO: 946 - DNA, 24 bp, Artificial
  probe
actcacacagaagtggtttcccgc

SEQ ID NO: 947 - DNA, 24 bp, Artificial
  probe
tctctgggagcaaaggacagtcgt

SEQ ID NO: 948 - DNA, 24 bp, Artificial
  probe
gcttctaacctaggaggctggtct

SEQ ID NO: 949 - DNA, 24 bp, Artificial
  probe
tgtggatgggactatgattcgcaa

SEQ ID NO: 950 - DNA, 24 bp, Artificial
  probe
tgacttccacacaaccactgagct

SEQ ID NO: 951 - DNA, 24 bp, Artificial
  probe
tcgtaccatcagcaggaaccaagc

SEQ ID NO: 952 - DNA, 24 bp, Artificial
  probe
accagcagcaatggcaattcagtg

SEQ ID NO: 953 - DNA, 24 bp, Artificial
  probe
tacacagccagcggcgttacatct

SEQ ID NO: 954 - DNA, 24 bp, Artificial
  probe
acaagtacaccaagcaagcaaggc

SEQ ID NO: 955 - DNA, 24 bp, Artificial
  probe
cctggtgctcatcggtaacactgg

SEQ ID NO: 956 - DNA, 24 bp, Artificial
  probe
acgtgttggagattgccagacagc

SEQ ID NO: 957 - DNA, 24 bp, Artificial
  probe
cagccaccatctcctagagtgagg

SEQ ID NO: 958 - DNA, 24 bp, Artificial
  probe
acaggttcatgttctctggctgca

SEQ ID NO: 959 - DNA, 24 bp, Artificial
  probe
agaaccgtcctccagtggtgaagc

SEQ ID NO: 960 - DNA, 24 bp, Artificial
  probe
tccagagtggtctcttctcaggcc

SEQ ID NO: 961 - DNA, 24 bp, Artificial
  probe
ccacatccagttccaccaccaatg

SEQ ID NO: 962 - DNA, 24 bp, Artificial
  probe
caggactcagcatgatggacagtg

SEQ ID NO: 963 - DNA, 24 bp, Artificial
  probe
cctgaggtgcacagagccaacatt

SEQ ID NO: 964 - DNA, 24 bp, Artificial
  probe
tgaggtgcacagagccaacattac

SEQ ID NO: 965 - DNA, 24 bp, Artificial
  probe
ccagttgcctagtgtccacctcct

SEQ ID NO: 966 - DNA, 24 bp, Artificial
  probe
atgcaggctcacttttccacatgt

SEQ ID NO: 967 - DNA, 24 bp, Artificial
  probe
cacttgctggctagtatgcacctg

SEQ ID NO: 968 - DNA, 24 bp, Artificial
  probe
atgaggaagtgctgatgtcgctgg

(19) United States
(12) Patent Application Publication
(10) Pub. No.: US 2017/0349951 A1
(43) Pub. Date: Dec. 7, 2017

(54) COMPOSITIONS AND METHODS FOR DIAGNOSING THYROID CANCER

(71) Applicant: DIAXONHIT, PARIS (FR)

(72) Inventors: PASCALE BEURDELEY, FONTENAY SOUS BOIS (FR); MICHAEL BRENNER, DAMASCUS, MD (US); ANNE-CHARLOTTE JARRIGE, PARIS (FR); DIEGO PALLARES, PARIS (FR)

(21) Appl. No.: 15/536,772

(22) PCT Filed: Dec. 16, 2015
(86) PCT No.: PCT/EP2015/080101

§ 371 Date: Jun. 16, 2017

(30) Foreign Application Priority Data
Dec. 17, 2014 (EP) EP14198610.9

(51) Int. Cl. C12Q 1/68

(52) U.S. Cl. CPC C12Q 1/6886; C12Q 2600/158; G01N 2800/046

(57) ABSTRACT

The present application relates to methods and compositions for detecting thyroid cancer in mammals, in particular in humans. The invention provides novel biomarkers which, in combination, allow effective, specific and sensitive detection of thyroid cancer, particularly to assist in the diagnosis of thyroid lesions in human subjects. The invention further relates to tools and/or kits (such as reagents, probes, primers, chips or arrays) suitable for implementing said methods, and the preparation and uses thereof.

The present application relates to methods and compositions for detecting thyroid cancer in mammals, in particular in human beings. The invention provides novel biomarkers which allow effective, specific and sensitive detection of thyroid cancer, particularly to assist in the diagnosis of thyroid lesions in human subjects. The invention further relates to tools and/or kits (such as reagents, probes, primers, chips or arrays) suitable for implementing said methods, and the preparation and uses thereof.

Thyroid cancer is the most common malignancy of the endocrine system (Curado, Edwards et al. 2007). Over 60 000 new cases are diagnosed each year in the US; despite its low malignancy potential, it is estimated that 1,890 patients will die of this disease in 2014 (http://seer.cancer.gov/statfacts/html/thyro.html accessed Oct. 15, 2014).

Three different types of thyroid cancer have been defined according to their histological features: differentiated thyroid cancer (DTC) deriving from epithelial cells from the thyroid follicles; medullary thyroid cancer (MTC); and anaplastic thyroid cancer (ATC). Approximately 90% of diagnosed thyroid cancers correspond to DTC, with papillary (PTC) histology being the most frequent (75%), followed by follicular (FTC) (10%), Hürthle cells (5%), and poorly differentiated carcinomas (1-6%). Overall, MTC accounts for approximately 10% of all thyroid tumors and ATC barely 1% [2]. Although the papillary thyroid cancer (PTC) is the most frequent, the follicular thyroid cancer (FTC) is less common but more aggressive. Hürthle cell thyroid cancer (HCC) is an uncommon but distinct and generally more aggressive type of thyroid cancer, which is now classified by the World Health Organization (WHO) as a subtype of follicular cancer (Harris and Bible 2011). Most DTC patients have a very good prognosis if diagnosed at early stages, with a 91% survival rate at 20 years when the classical treatment with surgery followed by suppression of thyroid stimulating hormone (TSH) is employed.

Thyroid nodules are a frequent clinical finding during thyroid examination with a prevalence ranging between 4 and 7% in the general population. This prevalence is even higher in case of ultrasonography of the cervical area and can reach 50% of patients over the age of 65. The most accurate tool for evaluating thyroid nodules is fine needle aspiration (FNA), with an accuracy >95%. There are several classification systems for thyroid cytology, the most recognized and the most widely used is the Bethesda classification (Cibas and Ali, 2009) composed of 6 categories for which the recommended patient management is different:
1. Bethesda category I: Non diagnostic (frequency: about 10%): the cytological analysis is not possible because, for instance, insufficient cells have been collected, the repeat of the FNA is recommended.
2. Cat II: Benign (frequency: about 70%): the malignancy rate is below 3%, the patients are referred to clinical follow up.
3. Cat III: Follicular lesion or neoplasm of unknown significance: the malignancy rate is of 5% to 15%, the patients are referred to clinical follow up and repeated FNA.
4. Cat IV: Follicular lesion or neoplasm: the malignancy rate is in the range of 15% to 30%. The recommended approach can include partial or complete thyroidectomy. The frequency of follicular neoplasms and atypia/follicular lesions of undetermined significance is about 15%.
5. Cat V: Suspicious of malignancy: the malignancy rate is of 60% to 75% and complete removal of the thyroid is recommended.
6. Cat VI: Malignant: the malignancy rate is 97% to 99%, the patient's thyroid gland should be resected.
   The frequency of category V and category VI (suspicion for malignancy and malignancy) is ≦5%.

Because FNA is unable to provide a definitive diagnosis for the nodules in categories 3, 4 and 5, most patients with indeterminate cytology undergo diagnostic surgery (i.e., partial thyroidectomy) to establish a histopathological diagnosis. If malignancy is identified, the patients require a complete thyroid resection. In contrast, if the pathological exam reveals a benign nodule, partial thyroidectomy has proven to be unnecessary (frequency: 60 to 80%). This situation of indeterminate cytology is encountered in up to 25% of the patients with a thyroid nodule undergoing FNA and shows the need of more powerful molecular tools to decrease the number of unnecessary surgeries.

Recent work has investigated the potential benefit of combining microscopic and mutation analyses (Nikiforov et al, 2011). There are several molecular tests available such as Thyroseq (Nikiforova et al, 2013) or Afirma (Alexander et al, 2012). These tests, however, have either low specificity or low sensitivity. Epigenetic and peripheral blood markers have also been studied but they have limited sensitivity and fail to detect 30% to 40% of cancers (Hu et al, 2006). WO2013/086429 provides long lists of genes presented as potentially related to thyroid cancer. Eszlinger et al (Endocrine reviews 28(3), 2007, 322) and Huang et al (PNAS 98(26), 2001, 15044) discuss gene expression profiling of thyroid tumors. A number of genes are listed in these documents but no hint towards specific combinations thereof is provided which would allow specific and effective thyroid cancer management.

There is a need for alternative methods of detecting thyroid cancer that are specific and sensitive, therefore increasing the number of definitive diagnoses and reducing unnecessary diagnostic surgeries.

### SUMMARY OF THE INVENTION

The present invention provides novel biomarkers which allow very specific and sensitive diagnosis of thyroid cancer. Starting from approximately 20 000 genes and 35 000 alternative splicing events, the inventors have been able to identify target genes as highly relevant biomarkers of thyroid cancer. By combining expression markers from these genes and/or domains of these genes, it is possible to determine with a high level of certainty, the malignant vs benign stage of thyroid lesions in human samples. The invention specifically improves the ability to distinguish malignant versus benign profiles within a population of FNA samples being considered as “indeterminate” (especially in categories III and IV from the Bethesda classification) during a cytological exam. By providing a determinant diagnosis to subjects initially identified as “indeterminate” the invention improves the clinical management of patients with thyroid nodules in order to avoid unnecessary thyroid surgeries.

An object of the invention more particularly relates to a method for aiding in the diagnosis of thyroid cancer in mammals, comprising measuring a set of target biomarkers in a biological sample from the mammal and diagnosing said thyroid cancer by comparing the measured value to a reference value, wherein the set of target biomarkers comprises an expression product of at least two genes selected among CITED1, TENM1, CCM2, SCLY and IGSF10 or at least two distinct domains of at least one expression product of a gene selected among CITED1, TENM1, CCM2, SCLY and IGSF10.

A further object of the invention relates to a method as defined above for determining whether the mammal has or is likely to have a malignant thyroid lesion.

Another object of the invention relates to a method for diagnosing thyroid cancer in mammals, comprising measuring an expression product of at least two genes selected among CITED1, TENM1, CCM2, SCLY and IGSF10, or at least two distinct domains of at least one expression product of a gene selected among CITED1, TENM1, CCM2, SCLY and IGSF10, in a biological sample from the mammal, and diagnosing said thyroid cancer by comparing the measured value to a reference value.

The invention further relates to the use of a set of target biomarkers comprising an expression product of at least two genes selected among CITED1, TENM1, CCM2, SCLY and IGSF10, or at least two distinct domains of at least one expression product of a gene selected among CITED1, TENM1, CCM2, SCLY and IGSF10, for the diagnosis in vitro or ex vivo of thyroid cancer.

In a preferred embodiment, the method comprises measuring in a biological sample from the subject an expression product of CITED1 and an expression product of TENM1. In a further preferred embodiment, the method comprises measuring in a biological sample from the subject an expression product of CITED1, TENM1, CCM2, SCLY and IGSF10 genes.

The above biomarkers may be further combined with additional biomarkers. In this regard, in a particular embodiment, the set of biomarkers further comprises an expression product of the FRMD3 gene.

In further particular embodiments of the invention, the set of biomarkers further comprises at least one protein or RNA encoded by a gene selected from Table A, preferably each gene listed in Table A.

In further particular embodiments of the invention, the set of biomarkers further comprises at least one protein or RNA encoded by a gene selected from Table B, preferably each gene listed in Table B.

In a particular embodiment of the invention, the set of biomarkers comprises one expression product of CITED1, TENM1, CCM2, SCLY, IGSF10 and FRMD3 and of each gene listed in Table A and in Table B.

In a specific mode of carrying out the invention, the method comprises:

a. providing a biological sample (comprising thyroid cells) from the subject having a thyroid condition or suspected to have a thyroid condition; and
b. measuring in said biological sample the expression of at least two genes selected among CITED1, TENM1, CCM2, SCLY and IGSF10, or at least two distinct domains of at least one expression product of a gene selected among CITED1, TENM1, CCM2, SCLY and IGSF10.

A further object of the invention resides in a kit comprising a solid support comprising at least two capture agents attached thereto, wherein each capture agent binds to an expression product of a distinct gene selected among CITED1, TENM1, CCM2, SCLY and IGSF10, or to at least two distinct domains of at least one expression product of a gene selected among CITED1, TENM1, CCM2, SCLY and IGSF10.

Another object of the invention relates to a method of treating thyroid cancer in a subject, the method comprising (i) measuring the expression product of at least 2 genes selected among CITED1, TENM1, CCM2, SCLY and IGSF10 in a sample from the subject, said measure allowing the diagnosis of thyroid cancer, and (ii) resecting the cancer when the subject is diagnosed to have thyroid cancer in step (i).

The invention may be used on any mammal, in particular any human subject. It is particularly effective to discriminate between malignant and benign thyroid lesions or nodules in a subject, and therefore to determine whether a thyroid lesion in a subject is malignant. The invention may be used on cellular samples, tissue biopsy or fine thyroid needle aspirate.

### LEGEND TO THE FIGURES

FIG. 1: ROC curves generated using a p-value cut-off of 10⁻³. A. Correlation with Malignant profile, B. Correlation with Benign profile and C. Difference between Correlation with Benign and Malignant profiles.

FIG. 2: Correlation of each specimen with the average benign and malignant profiles (cut-off 10⁻³).

FIG. 3: ROC curves generated using a p-value cut-off of 10⁻⁵. A. Correlation with Malignant profile, B. Correlation with Benign profile and C. Difference between Correlation with Benign and Malignant profiles.

FIG. 4: Correlation of each specimen with the average benign and malignant profiles (cut-off 10⁻⁵).

FIG. 5: ROC curves generated using a p-value cut-off of 10⁻⁷. A. Correlation with Malignant profile, B. Correlation with Benign profile and C. Difference between Correlation with Benign and Malignant profiles.

FIG. 6: Correlation of each specimen with the average benign and malignant profiles (cut-off 10⁻⁷).

### DETAILED DESCRIPTION OF THE INVENTION

The present invention provides novel methods for diagnosing thyroid cancer based on the analysis of new biomarkers. Upon comprehensive analysis of all of the human genes in biological samples from patients with malignant or benign nodules, the inventors identified genes which represent strong diagnostic biomarkers of thyroid cancer and may be used to determine, with high level of certainty, the malignant vs benign status of thyroid lesions in human subject samples. Expression of these gene products is strongly correlated to the disease and allows discrimination between malignant and benign nodules of the thyroid gland based on only two genes or on only two distinct domains of one of said genes.

An object of the invention more particularly relates to a method for (aiding in) the diagnosis of thyroid cancer in mammals, comprising the measurement of an expression product of at least two genes selected among CITED1, TENM1, CCM2, SCLY and IGSF10 or of two distinct domains of one of said genes, in a sample from the mammal and the diagnosis of said thyroid cancer by comparing the measured value to a reference value.

Another object of the invention relates to a method for (aiding in) the diagnosis of thyroid cancer in mammals, comprising the measurement of the expression of at least two distinct domains of at least one, preferably at least 2 genes selected among CITED1, TENM1, CCM2, SCLY and IGSF10 in a sample from the mammal and the diagnosis of said thyroid cancer by comparing the measured value to a reference value.

In a particular embodiment, the method comprises:
a) providing a biological sample from a subject having a thyroid condition (e.g. a thyroid lesion or nodule) or suspected to have a thyroid condition; and
b) measuring in said biological sample the expression product of genes as defined above.

In a further particular embodiment, the invention is conducted after cytological exam and allows the diagnosis of patient samples remaining “indeterminate” after said cytological exam. In this regard, a particular method of the invention comprises:
a) the provision (e.g., the collection) of a biological sample from the subject having a thyroid condition or suspected to have a thyroid condition;
b) a cytological exam of the sample to classify the thyroid condition (in a category comprised between 1 and 6) according to the Bethesda classification, and
c) for any sample of indeterminate cytology, the measurement of the expression product of target genes as described above.

Target Genes

The invention discloses the identification of genes whose expression products allow efficient diagnosis of thyroid cancer. Said genes have been selected from a set of approximately 20,000 human genes and demonstrate a greater than 97% specificity in a thyroid cancer diagnostic, which is remarkable. These genes, in various combinations, are sufficient to produce strong diagnostic results of thyroid cancer, which is very advantageous when compared with current proposed tests comprised of more than 100 genes.

Preferred target genes for use in the invention are CITED1, TENM1, CCM2, SCLY and IGSF10. As disclosed in the examples, detecting a gene product of at least 2 of these genes, or at least two domains of a gene product of one of these genes, is sufficient to provide a reliable diagnosis of thyroid cancer.

The full length nucleic acid sequences of these genes are publicly available and may be found in the NCBI Entrez gene database. The identifications for each of these genes is provided below as well as in Table C:

| Entrez | Gene | |
|---|---|---|
| Gene ID | name | Gene description |
| 4435 | CITED1 | Cbp/p300-interacting transactivator, with |
| | | Glu/Asp-rich carboxy-terminal domain, 1 |
| 10178 | TENM1 | odz, odd Oz/ten-m homolog 1(Drosophila) |
| 51540 | SCLY | selenocysteine lyase |
| 83605 | CCM2 | cerebral cavernous malformation 2 |
| 257019 | FRMD3 | FERM domain containing 3 |
| 285313 | IGSF10 | immunoglobulin superfamily, member 10 |

Within the context of the present invention, the term “CITED1 gene” designates preferably a human CITED1 gene, particularly a nucleic acid molecule or sequence comprising (i) a sequence of gene#4435 or a sequence complementary thereto; or (ii) a natural variant of a sequence of (i) such as a polymorphism; or (iii) a sequence having at least 90% identity, preferably at least 95, 96, 97, 98 or 99%, to a sequence of (i) or (ii); or (iv) a sequence hybridizing under stringent conditions to a sequence of (i) or (ii).

The same definition applies to the TENM1, CCM2, SCLY and IGSF10 genes by reference to gene#10178, 83605, 51540 and 285313, respectively, as well as to any other gene quoted in the present application, by reference to the corresponding reference sequence (see Tables A to C).

The present invention shows that the above genes or alternative isoforms of those genes are deregulated in thyroid cancer subjects so that any domain or sequence of an expression product encoded by said genes may be measured. For improved detection, however, especially when the method is based on a combined measure of the expression product of only 1 to 3 of said genes, the invention shows that particular domains or sequences (“target sequences”) may be targeted in the expression products of these genes for improved thyroid cancer diagnosis.

In this regard, in relation to CITED1 gene, the method preferably comprises measuring (e.g., determining the presence, absence or amount of) at least one sequence encoded by exon4, exon3, or exon(−2) in an expression product of the CITED1 gene. As shown in the experimental section, by designing reagents (e.g., probes or primers) that detect such preferred domains of CITED1 gene products, a more specific and sensitive detection of thyroid cancer is obtained. In a more preferred embodiment, the method comprises measuring (e.g., determining the presence, absence or amount of) a target sequence of CITED1 comprised in any of the sequences listed as SEQ ID NOs: 106-119.

With regard to TENM1, in a particular embodiment, the method comprises measuring (e.g., determining the presence, absence or amount of) at least one sequence encoded by exon1, 3, 13, 15, 17, 18, 19, 25 or 32 in an expression product of the TENM1 gene. As shown in the experimental section, by designing reagents that detect such preferred domains of TENM1 gene products, a more specific and sensitive detection of thyroid cancer is obtained. In a more preferred embodiment, the method comprises measuring (e.g., determining the presence, absence or amount of) a target sequence of TENM1 comprised in any of the sequences listed as SEQ ID NOs: 817-838.

With regard to CCM2, in a particular embodiment, the method comprises measuring a target sequence comprised in SEQ ID NO: 83.

With regard to SCLY, in a particular embodiment, the method comprises measuring a target sequence comprised in SEQ ID NO: 749.

With regard to IGSF10, in a particular embodiment, the method comprises measuring a target sequence comprised in SEQ ID NO: 335.

Moreover, in a further particular embodiment, the invention comprises measuring at least two distinct target sequence of an expression product the above target genes. The invention indeed shows that by monitoring two or more distinct domains in e.g., CITED1 or TENM1 gene products, an efficient and reliable diagnosis of thyroid cancer is made.

In this regard, in a particular embodiment, the method comprises measuring at least one sequence encoded by exon4 of the CITED1 gene and at least one sequence encoded by exon 3 and/or exon(−2) of the CITED1 gene.

In another particular embodiment, the method comprises measuring at least one sequence encoded by exon17, 18, 19, 25, or 32 of the TENM1 gene and at least one sequence encoded by exon1, 3, 13 or 15 of the TENM1 gene.

The results shown in the experimental section demonstrate that, by detecting distinct target sequences or domains of a single target gene as defined above, a very reliable detection of thyroid cancer is obtained. For instance, by detecting 3 domains of CITED1, a sensitivity and specificity of 75% and 90%, respectively, can be achieved, and by detecting several distinct target sequences as defined above of CITED1 and TENM1, a specificity of 92.5% can be obtained, which is remarkable and suitable to design a commercial test.

In a preferred embodiment, the invention comprises measuring a gene product of at least the CITED1 and TENM1 genes.

In another embodiment, the invention comprises measuring a gene product of at least the CITED1 and CCM2 genes.

In another embodiment, the invention comprises measuring a gene product of at least the CITED1 and SCLY genes.

In another embodiment, the invention comprises measuring a gene product of at least the CITED1, TENM1, CCM2, SCLY and IGSF10 genes.

Furthermore, while the above 5 target genes already provide highly reliable test, it is possible to further improve the method by combining said genes with further marker genes identified by the inventors.

In this regard, in a particular and preferred embodiment, the method further comprises the determination of at least one gene product of the FRMD3 gene (gene#257019). In a particular embodiment, for measuring FRMD3 gene expression, the method comprises measuring a target sequence comprised in any of the sequences listed as SEQ ID NOs: 249 to 269.

In a preferred embodiment, the invention comprises measuring a gene product of at least the CITED1 and FRMD3 genes.

In another embodiment, the invention comprises measuring a gene product of at least the CITED1 and TENM1 and FRMD3 genes.

In another embodiment, the invention comprises measuring a gene product of at least the CITED1, TENM1, CCM2, SCLY, IGSF10, and FRMD3 genes.

Moreover, to design more universal tests, the invention combines additional marker(s) identified by the inventors, selected from the expression products of the genes listed in table A and/or in Table B below:

TABLE A

| Entrez | | |
|---|---|---|
| Gene | | |
| ID | Gene name | Gene description |
| 607 | BCL9 | B-cell CLL/lymphoma 9 |
| 954 | ENTPD2 | ectonucleoside triphosphate diphosphohydrolase 2 |
| 956 | ENTPD3 | ectonucleoside triphosphate diphosphohydrolase 3 |
| 1000 | CDH2 | cadherin 2, type 1, N-cadherin (neuronal) |
| 1756 | DMD | dystrophin |
| 2059 | EPS8 | epidermal growth factor receptor pathway |
| | | substrate 8 |
| 2563 | GABRD | gamma-aminobutyric acid (GABA) A receptor, |
| | | delta |
| 3489 | IGFBP6 | insulin-like growth factor binding protein 6 |
| 4038 | LRP4 | low density lipoprotein receptor-related protein 4 |
| 4133 | MAP2 | microtubule-associated protein 2 |
| 5090 | PBX3 | pre-B-cell leukemia homeobox 3 |
| 6258 | RXRG | retinoid X receptor, gamma |
| 6271 | S100A1 | S100 calcium binding protein A1 |
| 6447 | SCG5 | secretogranin V (7B2 protein) |
| 6585 | SLIT1 | slit homolog 1 (Drosophila) |
| 6715 | SRD5A1 | steroid-5-alpha-reductase, alpha polypeptide 1 (3- |
| | | oxo-5 alpha-steroid delta 4-dehydrogenase |
| | | alpha 1) |
| 6920 | TCEA3 | transcription elongation factor A (SII), 3 |
| 7024 | TFCP2 | transcription factor CP2 |
| 7991 | TUSC3 | tumor suppressor candidate 3 |
| 10921 | RNPS1 | RNA binding protein S1, serine-rich domain |
| 22807 | IKZF2 | IKAROS family zinc finger 2 (Helios) |
| 23043 | TNIK | TRAF2 and NCK interacting kinase |
| 23248 | RPRD2 | regulation of nuclear pre-mRNA domain |
| | | containing 2 |
| 23362 | PSD3 | pleckstrin and Sec7 domain containing 3 |
| 25963 | TMEM87A | transmembrane protein 87A |
| 55164 | SHQ1 | SHQ1 homo log (S. cerevisiae) |
| 93035 | PKHD1L1 | polycystic kidney and hepatic disease 1 |
| | | (autosomal recessive)-like 1 |
| 120892 | LRRK2 | leucine-rich repeat kinase 2 |
| 134548 | SOWAHA | ankyrin repeat domain 43 |
| 143872 | ARHGAP42 | Rho GTPase activating protein 42 |
| 153090 | DAB2IP | DAB2 interacting protein |
| 164832 | LONRF2 | LON peptidase N-terminal domain and ring |
| | | finger 2 |
| 165215 | FAM171B | family with sequence similarity 171, member B |
| 200879 | LIPH | lipase, member H |

TABLE B

| Entrez | | |
|---|---|---|
| Gene ID | Gene name | Gene description |
| 35 | ACADS | acyl-CoA dehydrogenase, C-2 to C-3 short chain |
| 132 | ADK | adenosine kinase |
| 134 | ADORA1 | adenosine A1 receptor |
| 165 | AEBP1 | AE binding protein 1 |
| 334 | APLP2 | amyloid beta (A4) precursor-like protein 2 |
| 430 | ASCL2 | achaete-scute complex homolog 2 (Drosophila) |
| 627 | BDNF | brain-derived neurotrophic factor |
| 720 | C4A | complement component 4A (Rodgers blood |
| | | group) |
| 721 | C4B | complement component 4B (Chido blood group) |
| 770 | CA11 | carbonic anhydrase XI |
| 825 | CAPN3 | calpain 3, (p94) |
| 835 | CASP2 | caspase 2, apoptosis-related cysteine peptidase |
| 836 | CASP3 | caspase 3, apoptosis-related cysteine peptidase |
| 894 | CCND2 | cyclin D2 |
| 953 | ENTPD1 | ectonucleoside triphosphate diphosphohydrolase 1 |
| 961 | CD47 | CD47 molecule |
| 1001 | CDH3 | cadherin 3, type 1, P-cadherin (placental) |
| 1014 | CDH16 | cadherin 16, KSP-cadherin |
| 1188 | CLCNKB | chloride channel Kb |
| 1271 | CNTFR | ciliary neurotrophic factor receptor |
| 1281 | COL3A1 | collagen, type III, alpha 1 |
| 1290 | COL5A2 | collagen, type V, alpha 2 |
| 1465 | CSRP1 | cysteine and glycine-rich protein 1 |
| 1474 | CST6 | cystatin E/M |
| 1499 | CTNNB1 | catenin (cadherin-associated protein), beta 1, |
| | | 88 kDa |
| 1508 | CTSB | cathepsin B |
| 1611 | DAP | death-associated protein |
| 1717 | DHCR7 | 7-dehydrocholesterol reductase |
| 1728 | NQO1 | NAD(P)H dehydrogenase, quinone 1 |
| 1730 | DIAPH2 | diaphanous homolog 2 (Drosophila) |
| 1809 | DPYSL3 | dihydropyrimidinase-like 3 |
| 1837 | DTNA | dystrobrevin, alpha |
| 1846 | DUSP4 | dual specificity phosphatase 4 |
| 1877 | E4F1 | E4F transcription factor 1 |
| 1889 | ECE1 | endothelin converting enzyme 1 |
| 1893 | ECM1 | extracellular matrix protein 1 |
| 1958 | EGR1 | early growth response 1 |
| 1967 | EIF2B1 | eukaryotic translation initiation factor 2B, |
| | | subunit 1 alpha, 26 kDa |
| 2065 | ERBB3 | v-erb-b2 erythroblastic leukemia viral oncogene |
| | | homolog 3 (avian) |
| 2115 | ETV1 | ets variant 1 |
| 2119 | ETV5 | ets variant 5 |
| 2167 | FABP4 | fatty acid binding protein 4, adipocyte |
| 2202 | EFEMP1 | EGF containing fibulin-like extracellular matrix |
| | | protein 1 |
| 2542 | SLC37A4 | solute carrier family 37 (glucose-6-phosphate |
| | | transporter), member 4 |
| 2560 | GABRB1 | gamma-aminobutyric acid (GABA) A receptor, |
| | | beta 1 |
| 2561 | GABRB2 | gamma-aminobutyric acid (GABA) A receptor, |
| | | beta 2 |
| 2569 | GABRR1 | gamma-aminobutyric acid (GABA) receptor, rho 1 |
| 2581 | GALC | galactosylceramidase |
| 2595 | GANC | glucosidase, alpha; neutral C |
| 2635 | GBP3 | guanylate binding protein 3 |
| 2859 | GPR35 | G protein-coupled receptor 35 |
| 2869 | GRK5 | G protein-coupled receptor kinase 5 |
| 3014 | H2AFX | H2A histone family, member X |
| 3098 | HK1 | hexokinase 1 |
| 3249 | HPN | hepsin |
| 3476 | IGBP1 | immunoglobulin (CD79A) binding protein 1 |
| 3679 | ITGA7 | integrin, alpha 7 |
| 3682 | ITGAE | integrin, alpha E (antigen CD103, human |
| | | mucosal lymphocyte antigen 1; alpha |
| | | polypeptide) |
| 3685 | ITGAV | integrin, alpha V (vitronectin receptor, alpha |
| | | polypeptide, antigen CD51) |
| 3710 | ITPR3 | inositol 1,4,5-triphosphate receptor, type 3 |
| 3751 | KCND2 | potassium voltage-gated channel, Shal-related |
| | | subfamily, member 2 |
| 3977 | LIFR | leukemia inhibitory factor receptor alpha |
| 4000 | LMNA | lamin A/C |
| 4013 | VWA5A | von Willebrand factor A domain containing 5A |
| 4050 | LTB | lymphotoxin beta (TNF superfamily, member 3) |
| 4124 | MAN2A1 | mannosidase, alpha, class 2A, member 1 |
| 4128 | MAOA | monoamine oxidase A |
| 4233 | MET | met proto-oncogene (hepatocyte growth factor |
| | | receptor) |
| 4240 | MFGE8 | milk fat globule-EGF factor 8 protein |
| 4257 | MGST1 | microsomal glutathione S-transferase 1 |
| 4281 | MID1 | midline 1 (Opitz/BBB syndrome) |
| 4301 | MLLT4 | myeloid/lymphoid or mixed-lineage leukemia |
| | | (trithorax homolog, Drosophila); translocated to, 4 |
| 4524 | MTHFR | methylenetetrahydrofolate reductase (NAD(P)H) |
| 4628 | MYH10 | myosin, heavy chain 10, non-muscle |
| 4646 | MYO6 | myosin VI |
| 4664 | NAB1 | NGFI-A binding protein 1 (EGR1 binding |
| | | protein 1) |
| 4665 | NAB2 | NGFI-A binding protein 2 (EGR1 binding |
| | | protein 2) |
| 4753 | NELL2 | NEL-like 2 (chicken) |
| 4781 | NFIB | nuclear factor I/B |
| 4897 | NRCAM | neuronal cell adhesion molecule |
| 4907 | NT5E | 5′-nucleotidase, ecto (CD73) |
| 4915 | NTRK2 | neurotrophic tyrosine kinase, receptor, type 2 |
| 4926 | NUMA1 | nuclear mitotic apparatus protein 1 |
| 4967 | OGDH | oxoglutarate (alpha-ketoglutarate) |
| | | dehydrogenase (lipoamide) |
| 5053 | PAH | phenylalanine hydroxylase |
| 5066 | PAM | peptidylglycine alpha-amidating |
| | | monooxygenase |
| 5087 | PBX1 | pre-B-cell leukemia homeobox 1 |
| 5125 | PCSK5 | proprotein convertase subtilisin/kexin type 5 |
| 5162 | PDHB | pyruvate dehydrogenase (lipoamide) beta |
| 5164 | PDK2 | pyruvate dehydrogenase kinase, isozyme 2 |
| 5166 | PDK4 | pyruvate dehydrogenase kinase, isozyme 4 |
| 5255 | PHKA1 | phosphorylase kinase, alpha 1 (muscle) |
| 5270 | SERPINE2 | serpin peptidase inhibitor, clade E (nexin, |
| | | plasminogen activator inhibitor type 1), member 2 |
| 5315 | PKM | pyruvate kinase, muscle |
| 5362 | PLXNA2 | plexin A2 |
| 5365 | PLXNB3 | plexin B3 |
| 5371 | PML | promyelocytic leukemia |
| 5504 | PPP1R2 | protein phosphatase 1, regulatory (inhibitor) |
| | | subunit 2 |
| 5567 | PRKACB | protein kinase, cAMP-dependent, catalytic, beta |
| 5650 | KLK7 | kallikrein-related peptidase 7 |
| 5655 | KLK10 | kallikrein-related peptidase 10 |
| 5725 | PTBP1 | polypyrimidine tract binding protein 1 |
| 5747 | PTK2 | PTK2 protein tyrosine kinase 2 |
| 5786 | PTPRA | protein tyrosine phosphatase, receptor type, A |
| 5793 | PTPRG | protein tyrosine phosphatase, receptor type, G |
| 5873 | RAB27A | RAB27A, member RAS oncogene family |
| 5887 | RAD23B | RAD23 homolog B (S. cerevisiae) |
| 5921 | RASA1 | RAS p21 protein activator (GTPase activating |
| | | protein) 1 |
| 5981 | RFC1 | replication factor C (activator 1) 1, 145 kDa |
| 6196 | RPS6KA2 | ribosomal protein S6 kinase, 90 kDa, polypeptide 2 |
| 6284 | S100A13 | S100 calcium binding protein A13 |
| 6297 | SALL2 | sal-like 2 (Drosophila) |
| 6299 | SALL1 | sal-like 1 (Drosophila) |
| 6385 | SDC4 | syndecan 4 |
| 6415 | SEPW1 | selenoprotein W, 1 |
| 6441 | SFTPD | surfactant protein D |
| 6449 | SGTA | small glutamine-rich tetratricopeptide repeat |
| | | (TPR)-containing, alpha |
| 6569 | SLC34A1 | solute carrier family 34 (sodium phosphate), |
| | | member 1 |
| 6695 | SPOCK1 | sparc/osteonectin, cwcv and kazal-like domains |
| | | proteoglycan (testican) 1 |
| 6709 | SPTAN1 | spectrin, alpha, non-erythrocytic 1 (alpha-fodrin) |
| 6812 | STXBP1 | syntaxin binding protein 1 |
| 6843 | VAMP1 | vesicle-associated membrane protein 1 |
| | | (synaptobrevin 1) |
| 7033 | TFF3 | trefoil factor 3 (intestinal) |
| 7039 | TGFA | transforming growth factor, alpha |
| 7048 | TGFBR2 | transforming growth factor, beta receptor II |
| | | (70/80 kDa) |
| 7050 | TGIF1 | TGFB-induced factor homeobox 1 |
| 7064 | THOP1 | thimet oligopeptidase 1 |
| 7069 | THRSP | thyroid hormone responsive |
| 7088 | TLE1 | transducin-like enhancer of split 1 (E(sp1) |
| | | homolog, Drosophila) |
| 7159 | TP53BP2 | tumor protein p53 binding protein, 2 |
| 7164 | TPD52L1 | tumor protein D52-like 1 |
| 7224 | TRPC5 | transient receptor potential cation channel, |
| | | subfamily C, member 5 |
| 7262 | PHLDA2 | pleckstrin homology-like domain, family A, |
| | | member 2 |
| 7462 | LAT2 | linker for activation of T cells family, member 2 |
| 8170 | SLC14A2 | solute carrier family 14 (urea transporter), |
| | | member 2 |
| 8202 | NCOA3 | nuclear receptor coactivator 3 |
| 8321 | FZD1 | frizzled homolog 1 (Drosophila) |
| 8506 | CNTNAP1 | contactin associated protein 1 |
| 8557 | TCAP | titin-cap (telethonin) |
| 8645 | KCNK5 | potassium channel, subfamily K, member 5 |
| 8654 | PDE5A | phosphodiesterase 5A, cGMP-specific |
| 8714 | ABCC3 | ATP-binding cassette, sub-family C |
| | | (CFTR/MRP), member 3 |
| 8848 | TSC22D1 | TSC22 domain family, member 1 |
| 8869 | ST3GAL5 | ST3 beta-galactoside alpha-2,3-sialyltransferase 5 |
| 8909 | ENDOU | endonuclease, polyU-specific |
| 9076 | CLDN1 | claudin 1 |
| 9122 | SLC16A4 | solute carrier family 16, member 4 |
| | | (monocarboxylic acid transporter 5) |
| 9194 | SLC16A7 | solute carrier family 16, member 7 |
| | | (monocarboxylic acid transporter 2) |
| 9203 | ZMYM3 | zinc finger, MYM-type 3 |
| 9213 | XPR1 | xenotropic and polytropic retrovirus receptor 1 |
| 9223 | MAGI1 | membrane associated guanylate kinase, WW and |
| | | PDZ domain containing 1 |
| 9249 | DHRS3 | dehydrogenase/reductase (SDR family) member 3 |
| 9353 | SLIT2 | slit homolog 2 (Drosophila) |
| 9414 | TJP2 | tight junction protein 2 (zona occludens 2) |
| 9455 | HOMER2 | homer homolog 2 (Drosophila) |
| 9617 | MTRF1 | mitochondrial translational release factor 1 |
| 9688 | NUP93 | nucleoporin 93 kDa |
| 9697 | TRAM2 | translocation associated membrane protein 2 |
| 9882 | TBC1D4 | TBC1 domain family, member 4 |
| 9902 | MRC2 | mannose receptor, C type 2 |
| 9931 | HELZ | helicase with zinc finger |
| 9969 | MED13 | mediator complex subunit 13 |
| 10205 | MPZL2 | myelin protein zero-like 2 |
| 10241 | CALCOCO2 | calcium binding and coiled-coil domain 2 |
| 10351 | ABCA8 | ATP-binding cassette, sub-family A (ABC1), |
| | | member 8 |
| 10454 | TAB1 | TGF-beta activated kinase 1/MAP3K7 binding |
| | | protein 1 |
| 10551 | AGR2 | anterior gradient homolog 2 (Xenopus laevis) |
| 10577 | NPC2 | Niemann-Pick disease, type C2 |
| 10585 | POMT1 | protein-O-mannosyltransferase 1 |
| 10810 | WASF3 | WAS protein family, member 3 |
| 10962 | MLLT11 | myeloid/lymphoid or mixed-lineage leukemia |
| | | (trithorax homolog, Drosophila); translocated to, |
| | | 11 |
| 11098 | PRSS23 | protease, serine, 23 |
| 11123 | RCAN3 | RCAN family member 3 |
| 11216 | AKAP10 | A kinase (PRKA) anchor protein 10 |
| 22925 | PLA2R1 | phospholipase A2 receptor 1, 180 kDa |
| 23015 | GOLGA8A | golgin A8 family, member A |
| 23052 | ENDOD1 | endonuclease domain containing 1 |
| 23077 | MYCBP2 | MYC binding protein 2 |
| 23081 | KDM4C | lysine (K)-specific demethylase 4C |
| 23140 | ZZEF1 | zinc finger, ZZ-type with EF-hand domain 1 |
| 23154 | NCDN | neurochondrin |
| 23211 | ZC3H4 | zinc finger CCCH-type containing 4 |
| 23220 | DTX4 | deltex homolog 4 (Drosophila) |
| 23250 | ATP11A | ATPase, class VI, type 11A |
| 23345 | SYNE1 | spectrin repeat containing, nuclear envelope 1 |
| 23348 | DOCK9 | dedicator of cytokinesis 9 |
| 23355 | VPS8 | vacuolar protein sorting 8 homolog (S. cerevisiae) |
| 23493 | HEY2 | hairy/enhancer-of-split related with YRPW |
| | | motif 2 |
| 23499 | MACF1 | microtubule-actin crosslinking factor 1 |
| 23514 | SPIDR | scaffolding protein involved in DNA repair |
| 23541 | SEC14L2 | SEC14-like 2 (S. cerevisiae) |
| 23635 | SSBP2 | single-stranded DNA binding protein 2 |
| 25797 | QPCT | glutaminyl-peptide cyclotransferase |
| 25825 | BACE2 | beta-site APP-cleaving enzyme 2 |
| 25833 | POU2F3 | POU class 2 homeobox 3 |
| 25915 | NDUFAF3 | NADH dehydrogenase (ubiquinone) 1 alpha |
| | | subcomplex, assembly factor 3 |
| 25976 | TIPARP | TCDD-inducible poly(ADP-ribose) polymerase |
| 25987 | TSKU | tsukushi small leucine rich proteoglycan |
| | | homolog (Xenopus laevis) |
| 26022 | TMEM98 | transmembrane protein 98 |
| 26034 | IPCEF1 | interaction protein for cytohesin exchange |
| | | factors 1 |
| 26224 | FBXL3 | F-box and leucine-rich repeat protein 3 |
| 26234 | FBXL5 | F-box and leucine-rich repeat protein 5 |
| 26297 | SERGEF | secretion regulating guanine nucleotide |
| | | exchange factor |
| 26470 | SEZ6L2 | seizure related 6 homolog (mouse)-like 2 |
| 26953 | RANBP6 | RAN binding protein 6 |
| 26960 | NBEA | neurobeachin |
| 27112 | FAM155B | family with sequence similarity 155, member B |
| 27241 | BBS9 | Bardet-Biedl syndrome 9 |
| 27344 | PCSK1N | proprotein convertase subtilisin/kexin type 1 |
| | | inhibitor |
| 28965 | SLC27A6 | solute carrier family 27 (fatty acid transporter), |
| | | member 6 |
| 29087 | THYN1 | thymocyte nuclear protein 1 |
| 29781 | NCAPH2 | non-SMC condensin II complex, subunit H2 |
| 29798 | C2ORF27A | chromosome 2 open reading frame 27A |
| 29842 | TFCP2L1 | transcription factor CP2-like 1 |
| 50509 | COL5A3 | collagen, type V, alpha 3 |
| 51115 | RMDN1 | regulator of microtubule dynamics 1 |
| 51195 | RAPGEFL1 | Rap guanine nucleotide exchange factor (GEF)- |
| | | like 1 |
| 51310 | SLC22A17 | solute carrier family 22, member 17 |
| 51330 | TNFRSF12A | tumor necrosis factor receptor superfamily, |
| | | member 12A |
| 51364 | ZMYND10 | zinc finger, MYND-type containing 10 |
| 51377 | UCHL5 | ubiquitin carboxyl-terminal hydrolase L5 |
| 51479 | ANKFY1 | ankyrin repeat and FYVE domain containing 1 |
| 51566 | ARMCX3 | armadillo repeat containing, X-linked 3 |
| 51704 | GPRC5B | G protein-coupled receptor, family C, group 5, |
| | | member B |
| 51809 | GALNT7 | UDP-N-acetyl-alpha-D- |
| | | galactosamine:polypeptide N- |
| | | acetylgalactosaminyltransferase 7 (GalNAc-T7) |
| 53405 | CLIC5 | chloride intracellular channel 5 |
| 53905 | DUOX1 | dual oxidase 1 |
| 54103 | GSAP | gamma-secretase activating protein |
| 54344 | DPM3 | dolichyl-phosphate mannosyltransferase |
| | | polypeptide 3 |
| 54470 | ARMCX6 | armadillo repeat containing, X-linked 6 |
| 54471 | MIEF1 | mitochondrial elongation factor 1 |
| 54532 | USP53 | ubiquitin specific peptidase 53 |
| 54587 | MXRA8 | matrix-remodelling associated 8 |
| 55129 | ANO10 | anoctamin 10 |
| 55220 | KLHDC8A | kelch domain containing 8A |
| 55294 | FBXW7 | F-box and WD repeat domain containing 7 |
| 55339 | WDR33 | WD repeat domain 33 |
| 55450 | CAMK2N1 | calcium/calmodulin-dependent protein kinase II |
| | | inhibitor 1 |
| 55521 | TRIM36 | tripartite motif containing 36 |
| 55558 | PLXNA3 | plexin A3 |
| 55650 | PIGV | phosphatidylinositol glycan anchor biosynthesis, |
| | | class V |
| 55652 | SLC48A1 | solute carrier family 48 (heme transporter), |
| | | member 1 |
| 55740 | ENAH | enabled homolog (Drosophila) |
| 55755 | CDK5RAP2 | CDK5 regulatory subunit associated protein 2 |
| 55813 | UTP6 | UTP6, small subunit (SSU) processome |
| | | component, homolog (yeast) |
| 56171 | DNAH7 | dynein, axonemal, heavy chain 7 |
| 56654 | NPDC1 | neural proliferation, differentiation and control, 1 |
| 56848 | SPHK2 | sphingosine kinase 2 |
| 56928 | SPPL2B | signal peptide peptidase-like 2B |
| 56999 | ADAMTS9 | ADAM metallopeptidase with thrombospondin |
| | | type 1 motif, 9 |
| 57121 | LPAR5 | lysophosphatidic acid receptor 5 |
| 57154 | SMURF1 | SMAD specific E3 ubiquitin protein ligase 1 |
| 57184 | FAM219B | chromosome 15 open reading frame 17 |
| 57185 | NIPAL3 | NIPA-like domain containing 3 |
| 57214 | CEMIP | cell migration inducing protein, hyaluronan |
| | | binding |
| 57474 | ZNF490 | zinc finger protein 490 |
| 57477 | SHROOM4 | shroom family member 4 |
| 57493 | HEG1 | HEG homolog 1 (zebrafish) |
| 57639 | CCDC146 | coiled-coil domain containing 146 |
| 57664 | PLEKHA4 | pleckstrin homology domain containing, family |
| | | A (phosphoinositide binding specific) member 4 |
| 58480 | RHOU | ras homolog gene family, member U |
| 60314 | C12ORF10 | chromosome 12 open reading frame 10 |
| 63891 | RNF123 | ring finger protein 123 |
| 64091 | POPDC2 | popeye domain containing 2 |
| 64478 | CSMD1 | CUB and Sushi multiple domains 1 |
| 64769 | MEAF6 | MYST/Esa1-associated factor 6 |
| 65986 | ZBTB10 | zinc finger and BTB domain containing 10 |
| 79143 | MBOAT7 | membrane bound O-acyltransferase domain |
| | | containing 7 |
| 79159 | NOL12 | nucleolar protein 12 |
| 79365 | BHLHE41 | basic helix-loop-helix family, member e41 |
| 79745 | CLIP4 | CAP-GLY domain containing linker protein |
| | | family, member 4 |
| 79852 | EPHX3 | epoxide hydrolase 3 |
| 79856 | SNX22 | sorting nexin 22 |
| 79875 | THSD4 | thrombospondin, type I, domain containing 4 |
| 79897 | RPP21 | ribonuclease P/MRP 21 kDa subunit |
| 79968 | WDR76 | WD repeat domain 76 |
| 80003 | PCNXL2 | pecanex-like 2 (Drosophila) |
| 80714 | PBX4 | pre-B-cell leukemia homeobox 4 |
| 81847 | RNF146 | ring finger protein 146 |
| 81849 | ST6GALNAC5 | ST6 (alpha-N-acetyl-neuraminyl-2,3-beta- |
| | | galactosyl-1,3)-N-acetylgalactosaminide alpha- |
| | | 2,6-sialyltransferase 5 |
| 83660 | TLN2 | talin 2 |
| 83891 | SNX25 | sorting nexin 25 |
| 84220 | RGPD5 | RANBP2-like and GRIP domain containing 5 |
| 84239 | ATP13A4 | ATPase type 13A4 |
| 84417 | C2ORF40 | chromosome 2 open reading frame 40 |
| 84527 | ZNF559 | zinc finger protein 559 |
| 84561 | SLC12A8 | solute carrier family 12 (potassium/chloride |
| | | transporters), member 8 |
| 84705 | GTPBP3 | GTP binding protein 3 (mitochondrial) |
| 84954 | MPND | MPN domain containing |
| 89797 | NAV2 | neuron navigator 2 |
| 89866 | SEC16B | SEC16 homolog B (S. cerevisiae) |
| 89927 | C16ORF45 | chromosome 16 open reading frame 45 |
| 90161 | HS6ST2 | heparan sulfate 6-O-sulfotransferase 2 |
| 90231 | KIAA2013 | KIAA2013 |
| 90627 | STARD13 | StAR-related lipid transfer (START) domain |
| | | containing 13 |
| 91775 | NXPE3 | family with sequence similarity 55, member C |
| 92014 | SLC25A51 | mitochondrial carrier triple repeat 1 |
| 93611 | FBXO44 | F-box protein 44 |
| 113201 | CASC4 | cancer susceptibility candidate 4 |
| 113444 | SMIM12 | small integral membrane protein 12 |
| 114800 | CCDC85A | coiled-coil domain containing 85A |
| 115353 | LRRC42 | leucine rich repeat containing 42 |
| 122786 | FRMD6 | FERM domain containing 6 |
| 124056 | NOXO1 | NADPH oxidase organizer 1 |
| 125206 | SLC5A10 | solute carrier family 5 (sodium/glucose |
| | | cotransporter), member 10 |
| 126133 | ALDH16A1 | aldehyde dehydrogenase 16 family, member A1 |
| 129285 | PPP1R21 | KLRAQ motif containing 1 |
| 140469 | MYO3B | myosin IIIB |
| 140688 | NOL4L | nucleolar protein 4-like |
| 146439 | CCDC64B | coiled-coil domain containing 64B |
| 149175 | MANEAL | mannosidase, endo-alpha-like |
| 149461 | CLDN19 | claudin 19 |
| 154467 | CCDC167 | chromosome 6 open reading frame 129 |
| 155054 | ZNF425 | zinc finger protein 425 |
| 158158 | RASEF | RAS and EF-hand domain containing |
| 163702 | IFNLR1 | interferon, lambda receptor 1 |
| 166929 | SGMS2 | sphingomyelin synthase 2 |
| 168391 | GALNTL5 | UDP-N-acetyl-alpha-D- |
| | | galactosamine:polypeptide N- |
| | | acetylgalactosaminyltransferase-like 5 |
| 196410 | METTL7B | methyltransferase like 7B |
| 202374 | STK32A | serine/threonine kinase 32A |
| 222171 | PRR15 | proline rich 15 |
| 253970 | SFTA3 | surfactant associated 3 |
| 283208 | P4HA3 | prolyl 4-hydroxylase, alpha polypeptide III |
| 283316 | CD163L1 | CD163 molecule-like 1 |
| 284418 | FAM71E2 | family with sequence similarity 71, member E2 |
| 285180 | RUFY4 | RUN and FYVE domain containing 4 |
| 285533 | RNF175 | ring finger protein 175 |
| 286144 | TRIQK | chromosome 8 open reading frame 83 |
| 286256 | LCN12 | lipocalin 12 |
| 340595 | ZCCHC16 | zinc finger, CCHC domain containing 16 |
| 342184 | FMN1 | formin 1 |
| 373156 | GSTK1 | glutathione S-transferase kappa 1 |
| 386653 | IL31 | interleukin 31 |
| 400746 | NCMAP | chromosome 1 open reading frame 130 |
| 401474 | SAMD12 | sterile alpha motif domain containing 12 |
| 440270 | GOLGA8B | golgin A8 family, member B |
| 440335 | SMIM22 | small integral membrane protein 22 |
| 645121 | CCNI2 | cyclin I family, member 2 |
| 727936 | GXYLT2 | glucoside xylosyltransferase 2 |
| 728492 | SERF1B | small EDRK-rich factor 1B (centromeric) |
| 100131187 | TSTD1 | thiosulfate sulfurtransferase (rhodanese)-like |
| | | domain containing 1 |
| 100293534 | C4B_2 | complement component 4B (Chido blood |
| | | group), copy 2 |
| 100509091 | LOC100509091 | extracellular matrix protein 2-like |

For each gene included in Table A and in Table B, a most preferred sequence (or domain) to be targeted for measuring gene expression according to the invention is provided in Table C. Accordingly, measuring BCL9 preferably comprises measuring a target sequence comprised in anyone of the sequences listed as SEQ ID NOs: 52 to 54 in Table C; measuring ENTPD2 preferably comprises measuring a target sequence comprised in SEQ ID NO: 205 as listed in Table C, etc.

Specific combinations of target gene products are provided in the following table D, which combinations can provide strong specificity and sensitivity for detection of thyroid cancer:

TABLE D
Combinations of marker genes

| Signature # | Expression Product(s) |
|---|---|
| 1 | CITED1 (several exons) |
| 2 | CITED1 and TENM1 |
| 3 | TENM1, CITED1, CCM2, SCLY |
| 4 | CITED1, TENM1, CCM2, SCLY and |
| | IGSF10 |
| F | TENM1, CITED1, CCM2, SCLY, |
| | FRMD3, IGSF10 |
| D | TENM1, CITED1, CCM2, SCLY, |
| | FRMD3, IGSF10 + all genes of table A |
| B | TENM1, CITED1, CCM2, SCLY, |
| | FRMD3, IGSF10 + all genes of table |
| | A + all gene of Table B |

Methods for Measuring Gene Expression Products

Within the context of the invention, the “measure” of a gene or gene expression product designates a measure or determination of the presence, absence, amount or alteration of such gene product. More preferably, the term “measure” or “measuring” designates a determination of the presence or amount of a gene product or domain, or of the level of expression of a gene. The gene product may be a RNA (e.g., a messenger RNA) or a polypeptide (or protein).

In a first preferred embodiment, the gene product is an RNA (which may be converted during the method into a cDNA). Various techniques for detecting a nucleic acid (e.g., RNA) in a sample can be used in the present invention, such as for example northern blot, selective hybridisation, the use of supports covered with oligonucleotide probes, nucleic acid amplification such as for example RT-PCR, quantitative PCR or ligation-PCR, etc. These methods can include the use of a nucleic probe (for example an oligonucleotide) capable of detecting selectively or specifically the target nucleic acid in the sample. Amplification can be performed according to various methods known to the person skilled in the art, such as PCR, LCR, transcription mediated amplification (TMA), strand displacement amplification (SDA), NASBA, the use of allele specific oligonucleotides (ASO), allele specific amplification, RNAseq. Detection can also be made using, e.g., Southern blot, single-strand conformation analysis (SSCA), in situ hybridisation (e.g., FISH), gel migration, heteroduplex analysis, NextGen sequencing, etc.

According to a preferred embodiment, the method comprises determining the presence or absence or (relative) amount of an RNA encoded by a target gene as defined above, preferably by selective hybridisation or selective amplification.

Measure by Selective Hybridization

Selective hybridisation is typically performed using nucleic probes, preferably immobilised on a support, such as a solid or semi-solid support having at least one surface, flat or not, for immobilising nucleic probes. Such supports are, for example, a slide, bead, membrane, filter, column, plate, etc. They can be made out of any compatible material, such as in particular glass, silica, plastic, fiber, metal, polymer, etc. The nucleic probes can be any nucleic acid (DNA, RNA, PNA, etc.), preferably single-stranded, with a sequence specific to a target RNA or DNA molecule. The probes typically comprise from 5 to 400 bases, preferably from 8 to 200, more preferentially less than 100, and even more preferentially less than 75, 60, 50, 40 or even 30 bases. The probes can be synthetic oligonucleotides, produced on the basis of the sequences of the target genes, according to standard synthesis techniques. Such oligonucleotides typically comprise from 10 to 50 bases, preferably from 20 to 40, for example approximately 25 bases. In a particularly advantageous embodiment, so as to improve the detection of signal, several different oligonucleotides (or probes) are used to detect the same target molecule (transcribed from the amplification of the patient's RNA to produce either cRNA or cDNA) during hybridisation. This may include specific oligonucleotides of various regions of the same target sequence, or centred differently on a given region. Advantageously, use is made of probe sets comprising 1-3 probes, which can be overlapping or not, completely or partially, and which are specific of the same target molecule. Use can also be made of probe pairs, in which one member is paired perfectly with the target sequence, and the other presents a mismatch, thus making it possible to estimate background signal. Probes can be designed to hybridise with a region of an exon or an intron, or with an exon-exon or exon-intron junction region. Thus, the probes make it possible to reveal and to distinguish various alternative spliced isoforms.

In a preferred embodiment, use is made of probes whose sequence comprises all or part of a nucleic acid sequence selected from anyone of SEQ ID NOs: 1-918 or of a sequence complementary thereto. In a preferred mode, use is made of nucleic acid probes having a length between 15 and 50 bases, more preferentially between 15 and 40 bases, and whose sequence is identical to a fragment of a sequence selected from anyone of SEQ ID NOs: 1-918 or of a sequence complementary thereto. The probe can also be designed in the opposite orientation. Such probes constitute another object of the present application, as well as the use thereof for diagnosing thyroid cancer in a subject.

Specific examples of probes are provided in Table E. These probes have been designed to bind target sequences as disclosed in Table C. Further probes can be designed using information provided in the invention, directed specifically to each target sequence of Table C. Each probe as disclosed in Table E represents a specific object of the invention.

TABLE E

| | | | SEQ |
|---|---|---|---|
| Probe Set ID | Probe ID | Probe Sequence | ID # |
| HSX2_01064533 | HSX2_01064533-39 | CACCGCTGAGCTGCCGAGAGGAAG | 919 |
| | HSX2_01064533-104 | GCAGTTCTCGGTGAGGAGGAAGAG | 920 |
| HSX2_01064539 | HSX2_01064539-42 | TCCAACTTCTGCCAAGGCTCTGAA | 921 |
| | HSX2_01064539-77 | GTCGAGGCCTGCACTTGATGTCAA | 922 |
| HSX2_01064543 | HSX2_01064543-27 | GGCCTACTCCAACCTTGCGGTGAA | 923 |
| | HSX2_01064543-46 | GTGAAAGATCGCAAAGCAGTGGCC | 924 |
| HSX2_01064546 | HSX2_01064546-23 | CCTCGGGATCTCCAATAGGCTCTC | 925 |
| | HSX2_01064546-24 | CTCGGGATCTCCAATAGGCTCTCC | 926 |
| HSX2_01064547 | HSX2_01064547-31 | CACTTGCTGGCTAGTATGCACCTG | 927 |
| | HSX2_01064547-166 | TCACTCTCTCCTTCTGCTGGTGCC | 928 |
| HSX2_01064548 | HSX2_01064548-32 | ATGAGGAAGTGCTGATGTCGCTGG | 929 |
| | HSX2_01064548-127 | ACTGCGGACTTTCCATCTAGCTGC | 930 |
| HSX2_01477556 | HSX2_01477556-78 | GCAAACCCTACCAGCCTCTACCAA | 931 |
| | HSX2_01477556-189 | GGGAGACCCTGCACGAGTATAACC | 932 |
| HSX2_01477563 | HSX2_01477563-15 | CCTGTTTGTTGTGACATGGAGGCT | 933 |
| | HSX2_01477563-23 | TTGTGACATGGAGGCTCAAGCTGG | 934 |
| HSX2_01477568 | HSX2_01477568-13 | GCCCACACAACCAGTTCACCTTCA | 935 |
| | HSX2_01477568-174 | AGCACGCAGGATTCAGTCCATCTG | 936 |
| HSX2_01477573 | HSX2_01477573-25 | TGGTTCCTCTGCGATCTTCAGTGC | 937 |
| | HSX2_01477573-189 | GCGCCACTGCAATCACAGTGACTT | 938 |
| HSX2_01477615 | HSX2_01477615-1 | CTCACTACTTAGATGCTGTCCGAG | 939 |
| | HSX2_01477615-1 | CTCACTACTTAGATGCTGTCCGAG | 940 |
| HSX2_01477625 | HSX2_01477625-72 | GGCTCACCAGATCCTCTTGACCTC | 941 |
| | HSX2_01477625-106 | GCCAAACTCTCTTCTCTCAGCACA | 942 |
| HSX2_01477635 | HSX2_01477635-73 | GAGAACACTCTGGTTGCCTTGGAA | 943 |
| | HSX2_01477635-192 | TGCTTCCTTCACCGCTCACATCAT | 944 |
| HSX2_01477640 | HSX2_01477640-81 | CCTGCTACGGATCCTTCTGACACA | 945 |
| | HSX2_01477640-162 | ACTCACACAGAAGTGGTTTCCCGC | 946 |
| HSX2_01477645 | HSX2_01477645-41 | TCTCTGGGAGCAAAGGACAGTCGT | 947 |
| | HSX2_01477645-87 | GCTTCTAACCTAGGAGGCTGGTCT | 948 |
| HSX2_01477665 | HSX2_01477665-35 | TGTGGATGGGACTATGATTCGCAA | 949 |
| | HSX2_01477665-106 | TGACTTCCACACAACCACTGAGCT | 950 |
| HSX2_01477675 | HSX2_01477675-104 | TCGTACCATCAGCAGGAACCAAGC | 951 |
| | HSX2_01477675-288 | ACCAGCAGCAATGGCAATTCAGTG | 952 |
| HSX2_01477695 | HSX2_01477695-17 | TACACAGCCAGCGGCGTTACATCT | 953 |
| | HSX2_01477695-242 | ACAAGTACACCAAGCAAGCAAGGC | 954 |
| HSX2_01477710 | HSX2_01477710-333 | CCTGGTGCTCATCGGTAACACTGG | 955 |
| | HSX2_01477710-515 | ACGTGTTGGAGATTGCCAGACAGC | 956 |
| HSX2_01477711 | HSX2_01477711-469 | CAGCCACCATCTCCTAGAGTGAGG | 957 |
| | HSX2_01477711-4356 | ACAGGTTCATGTTCTCTGGCTGCA | 958 |
| HSX2_01771392 | HSX2_01771392-460 | AGAACCGTCCTCCAGTGGTGAAGC | 959 |
| | HSX2_01771392-673 | TCCAGAGTGGTCTCTTCTCAGGCC | 960 |
| HSX2_02073060 | HSX2_02073060-1 | CCACATCCAGTTCCACCACCAATG | 961 |
| | HSX2_02073060-141 | CAGGACTCAGCATGATGGACAGTG | 962 |
| HSX2_02319619 | HSX2_02319619-4 | CCTGAGGTGCACAGAGCCAACATT | 963 |
| | HSX2_02319619-6 | TGAGGTGCACAGAGCCAACATTAC | 964 |
| HSX2_02487762 | HSX2_02487762-163 | CCAGTTGCCTAGTGTCCACCTCCT | 965 |
| | HSX2_02487762-422 | ATGCAGGCTCACTTTTCCACATGT | 966 |
| HSX2_02509166 | HSX2_02509166-208 | CACTTGCTGGCTAGTATGCACCTG | 967 |
| | HSX2_02509166-401 | ATGAGGAAGTGCTGATGTCGCTGG | 968 |

An object of the invention thus relates to a nucleic acid having anyone of the nucleotide sequences as described in Table E, as well as to the use of these nucleic acids for diagnosing thyroid cancer.

The probes can be synthesised in advance and then deposited on the support, or synthesised directly in situ, on the support, according to methods known to the person skilled in the art. The probes can also be manufactured by genomic or molecular techniques, for example by amplification, recombination, ligation, etc.

The probes can by hybridized with nucleic acids, preferably RNAs contained in test samples under standard, conventional conditions allowing specific hybridization to occur. Hybridisation can be performed under standard conditions, known to and adjustable by the person skilled in the art (see Sambrook, Fritsch, Maniatis (1989) Molecular Cloning, Cold Spring Harbor Laboratory Press). Most notably, hybridisation can be performed under conditions of high, medium or low stringency, according to the level of sensitivity needed, the quantity of material available, etc. For example, suitable hybridisation conditions include a temperature between 55° C. and 63° C. for 2 hours to 18 hours. Other hybridisation conditions, adapted to high density supports, are for example a hybridisation temperature between 45° C. and 55° C. After hybridisation, various washes can be performed to eliminate the non-hybridised molecules, typically in SSC buffers including SDS, such as a buffer comprising 0.1× to 10×SSC and 0.5% to 0.01% SDS. Other wash buffers containing SSPE, MES, NaCl or EDTA can also be used.

In a typical embodiment, the nucleic acids (or arrays or supports) are pre-hybridised in hybridisation buffer (Rapid Hybrid Buffer, Amersham) typically containing 100 μg/ml of salmon sperm DNA at 65° C. for 30 min. The nucleic acids of the sample are then placed in contact with the probes (typically applied to the support or the array) at 65° C. for 2 hours to 18 hours. Preferably, the nucleic acids of the sample are marked beforehand by any known marker (biotin, radioactive, enzymatic, fluorescent, luminescent, etc.). The supports are then washed in 5×SSC, 0.1% SDS buffer at 65° C. for 30 min., then in a 0.2×SSC, 0.1% SDS buffer. The expression profile is analysed according to standard techniques, such as for example by measuring labelling on the support by means of a suitable instrument (for example InstantImager, Packard Instruments). Hybridisation conditions naturally can be adjusted by the person skilled in the art, for example by modifying hybridisation temperature and/or the salt concentration of the buffer as well as by adding auxiliary substances such as formamide or single-strand DNA.

A particular object of the invention thus resides in a method to diagnose thyroid cancer in mammals comprising contacting, under conditions allowing hybridisation between complementary sequences to occur, (i) nucleic acids from a sample from the subject and (ii) a set of probes specific of target gene expression products as defined above; and diagnosing thyroid cancer by measuring hybridization to determine expression values and comparing said values to a reference value.

In a preferred embodiment, the method uses:
from 1 to 5 probes, preferably from 1 to 3, typically 2, overlapping or not, each comprising all or part of the sequence of CITED1 gene, preferably of anyone of the sequences listed as SEQ ID NOs: 106-119, or of a sequence complementary thereto; and
from 1 to 5 probes, preferably from 1 to 3, typically 2, overlapping or not, each comprising all or part of the sequence of TENM1 gene, preferably of anyone of the sequences listed as SEQ ID NOs: 817-838, or of a sequence complementary thereto,

wherein the term “part” advantageously indicates a region of 15 to 50 consecutive nucleotides, preferably of 15 to 30 nucleotides, even more preferably of 20-30 nucleotides.

In a more preferred embodiment, the method uses:
from 1 to 5 probes, preferably from 1 to 3, typically 2, overlapping or not, each comprising all or part of the sequence of CITED1 gene or of a sequence complementary thereto, preferably of anyone of the sequences listed as SEQ ID NOs: 106-119, or of a sequence complementary thereto;
from 1 to 5 probes, preferably from 1 to 3, typically 2, overlapping or not, each comprising all or part of the sequence of TENM1 gene or of a sequence complementary thereto, preferably of anyone of the sequences listed as SEQ ID NOs: 817-838, or of a sequence complementary thereto; and
from 1 to 5 probes, preferably from 1 to 3, typically 2, overlapping or not, each comprising all or part of the sequence of SCLY gene or of a sequence complementary thereto, preferably of SEQ ID NO: 749, or of a sequence complementary thereto;

wherein the term “part” advantageously indicates a region as defined above.

In a particularly preferred embodiment, the method uses:
from 1 to 5 probes, preferably from 1 to 3, typically 2, overlapping or not, each comprising all or part of the sequence of CITED1 gene or of a sequence complementary thereto, preferably of anyone of the sequences listed as SEQ ID NOs: 106-119, or of a sequence complementary thereto;
from 1 to 5 probes, preferably from 1 to 3, typically 2, overlapping or not, each comprising all or part of the sequence of TENM1 gene or of a sequence complementary thereto, preferably of anyone of the sequences listed as SEQ ID NOs: 817-838, or of a sequence complementary thereto;
from 1 to 5 probes, preferably from 1 to 3, typically 2, overlapping or not, each comprising all or part of the sequence of SCLY gene or of a sequence complementary thereto, preferably of SEQ ID NO: 749, or of a sequence complementary thereto;
from 1 to 5 probes, preferably from 1 to 3, typically 2, overlapping or not, each comprising all or part of the sequence of CCM2 gene or of a sequence complementary thereto, preferably of SEQ ID NO: 83, or of a sequence complementary thereto;
from 1 to 5 probes, preferably from 1 to 3, typically 2, overlapping or not, each comprising all or part of the sequence of IGSF10 gene or of a sequence complementary thereto, preferably of SEQ ID NO: 335, or of a sequence complementary thereto; and
from 1 to 5 probes, preferably from 1 to 3, typically 2, overlapping or not, each comprising all or part of the sequence of FRMD3 gene or of a sequence complementary thereto, preferably of anyone of the sequences listed as SEQ ID NOs: 249 to 269, or of a sequence complementary thereto;

wherein the term “part” advantageously indicates a region as defined above.

In another preferred embodiment, the method uses:
at least 1 probe that specifically hybridizes with exon 4 of the sequence of CITED1 gene or of a sequence complementary thereto; and
at least 1 probe that specifically hybridizes with exon 3 of the sequence of CITED1 gene or of a sequence complementary thereto and,
at least 1 probe that specifically hybridizes with exon(−2) of the sequence of CITED1 gene or of a sequence complementary thereto.

In another preferred embodiment, the method uses:
at least 1 probe that specifically hybridizes with exon1, 3, 13, or 15, of the sequence of TENM1 gene or of a sequence complementary thereto; and
at least 1 probe that specifically hybridizes with exon17, 18, 19, 25, or 32 of the sequence of TENM1 gene or of a sequence complementary thereto.

The invention may utilize additional probes specific for any group or combination of target genes of the invention, as preferably listed in the above table.

Measure by Selective Amplification

Selective amplification is preferably performed using a primer or a pair of primers to amplify all or part of one of the target nucleic acids in the sample, when the target nucleic acid is present. The primer can be specific for a target sequence according to anyone of SEQ ID NOs: 1 to 918, or for a region flanking the target sequences in a nucleic acid of the sample. The primer typically comprises a single-strand nucleic acid, with a length advantageously between 5 and 50 bases, preferably between 5 and 30 bases. Such a primer constitutes another object of the present application, as well as the use thereof (primarily in vitro) for diagnosing thyroid cancer in a subject. The primers can be designed to hybridise with a region of an exon or an intron, or with an exon-exon or exon-intron junction region. Thus, the primers reveal and distinguish various forms of gene splicing.

In this respect, another object of the invention resides in the use of a set of nucleotide primers comprising at least 2 pairs of primers, each pair of primers comprising a sense and/or antisense nucleic acid primers complementary to and specific of a target nucleic acid selected from anyone of the sequences listed as SEQ ID NOs: 1-918, or the complementary strand thereof, for in vitro or ex vivo diagnosis of thyroid cancer in a subject.

Another specific object of the invention resides in a method to diagnose thyroid cancer in mammals, comprising contacting, under conditions allowing amplification, nucleic acids from a sample from mammals and a set of primers comprising at least 2 pairs of primers, each pair of primers comprising nucleic acid primers complementary to and specific of a target nucleic acid selected from anyone of the sequences listed as SEQ ID NOs: 1-918 or the complementary strand thereof to obtain an amplification profile, and comparing said amplification profile to a reference value to diagnose thyroid cancer.

The invention also relates to a composition or kit comprising a set of primers comprising at least 2 pairs of primers, each pair of primers comprising a sense and/or antisense nucleic acid primers complementary to and specific of a target nucleic acid selected from anyone of the sequences listed as SEQ ID NOs: 1-918 or the complementary strand thereof.

Measuring Polypeptide

In another embodiment, the method comprises the measurement of a polypeptide encoded by a target gene such as defined previously. Measuring or assaying a polypeptide in a sample can be performed by any known technique, most notably by means of a specific ligand, for example an antibody or an antibody fragment or derivative. Preferably, the ligand is a specific antibody of the polypeptide, or a fragment of such an antibody (for example Fab, Fab′, CDR, etc.), or a derivative of such an antibody (for example a single-chain variable-fragment antibody, scFv). The ligand is typically immobilised on a support, such as a slide, bead, column, plate, etc. The presence or quantity of target polypeptide in the sample can be detected by revealing a complex between the target and the ligand, for example by using a labelled ligand or by using a second labelled indicator ligand, etc. Immunological techniques that can be used and are well known are ELISA, IHC and RIA techniques, etc. If necessary, the quantity of polypeptide detected can be compared to a reference value, for example a median or mean value observed among patients who do or do not have thyroid cancer, or with a value measured in parallel in a control sample. Thus, it is possible to demonstrate variation in expression levels.

Specific antibodies of target polypeptides can be produced by conventional techniques, most notably by immunization of a non-human mammal with an immunogen comprising the polypeptide (or an immunogenic fragment thereof), and recovery of the antibodies (polyclonal) or cells producing monoclonal antibodies. Production techniques for polyclonal or monoclonal antibodies, single-chain variable-fragment antibodies and human or humanised antibodies are described for example in Harlow et al., A Laboratory Manual, CSH Press, 1988; Ward et al., Nature 341 (1989) 544; Bird et al., Science 242 (1988) 423; WO 94/02602; U.S. Pat. No. 5,223,409; U.S. Pat. No. 5,877,293 and WO 93/01288. The immunogen can be produced by synthesis, or by expression, in a suitable host, of a nucleic acid target such as defined previously. Such antibodies, monoclonal or polyclonal, as well as derivatives with the same antigenic specificity, constitute further objects of the present application, as well as the use thereof to diagnose thyroid cancer.

Changes in protein expression and/or structure can also be detected by means of techniques, known by the person skilled in the art, involving mass spectroscopy, more generally grouped under the name proteome analysis.

Implementation of the Method

The method of the invention is applicable to any biological sample from the tested mammal, most notably any nature of sample that includes nucleic acids or polypeptides from a thyroid cell, most preferably from a thyroid lesion or nodule. Specific examples include any tissue, organ or biological fluid that includes nucleic acids or polypeptides from a thyroid cell, most preferably from a thyroid lesion or nodule. Most preferred samples are a tissue biopsy from thyroid, or a fine needle thyroid aspirate.

In one preferred and particularly advantageous embodiment, the sample is a sample collected by the physician for cytological analysis of the subject's thyroid lesion or nodules. Upon completion of such cytological analysis, if the status of the sample is indeterminate, then this sample may be used to perform the current test.

The sample can be obtained by any known technique, for example by drawing, by non-invasive techniques, or from sample collections or biobanks, etc. Further, the sample can be pre-treated to facilitate access to the target molecules, for example by lysis (mechanical, chemical, enzymatic, etc.), purification, centrifugation, separation, etc. The sample can also be labelled to facilitate the determination of the presence of target molecules (biotin, fluorescent, radioactive, luminescent, chemical or enzymatic labelling, etc.). The nucleic acids of the sample can in addition be separated, treated, enriched, purified, reverse transcribed, amplified, fragmented, etc. In a particular embodiment, the nucleic acids of the sample are RNAs, most notably mRNA of the sample. In a very specific embodiment, the nucleic acids are the product of amplification of RNA, most notably of mRNA, or cDNA prepared from RNA, most notably mRNA of the sample.

In a particular embodiment, the method comprises:
a) providing a biological sample from a subject having a thyroid condition (e.g. a thyroid lesion or nodule) or suspected to have a thyroid condition; and
b) measuring in said biological sample the expression product of genes as defined above.

In a further particular embodiment, the invention is conducted after a cytological exam and allows the diagnosis of patient samples remaining “indeterminate” after said cytological exam. In this regard, a particular method of the invention comprises:
a) the provision (e.g., the collection) of a biological sample from the subject having a thyroid condition or suspected to have a thyroid condition;
b) a cytological exam of the sample to classify the thyroid condition (e.g., in a category comprised between 1 and 6) according to the Bethesda classification, and
c) for any sample of indeterminate cytology, the measurement of the expression product of target genes as described above.

In a typical embodiment, the method measures a gene expression product and compares such expression signal to a reference value in order to diagnose thyroid cancer. In this regard, the reference value may be any control value, or any mean value in healthy subjects, or 2 reference values characteristic of a diseased and a non-diseased subject, respectively. In this regard, in a preferred embodiment of the invention, the measured value is compared to a reference value of a control benign thyroid condition and to a reference value of a control malignant thyroid condition, wherein, when the measured value is closer to the reference value of the control benign thyroid condition, then the tested thyroid condition is benign, and when the measured value is closer to the reference value of the control malignant thyroid condition, then the tested thyroid condition is malignant.

The invention is applicable to any mammal, preferably human beings. The data provided in the examples show that the invention can detect the presence of malignant or benign lesions when the cytology was unable to provide a diagnostic result. 60 samples (40 benign and 20 malignant lesions) were used to validate the relevance of the invention, which addresses a clinical unmet need and allows reduction or avoidance of unnecessary surgeries.

Another object of the present application relates to a product comprising a support on which are immobilised nucleic acids comprising a sequence complementary to and/or specific for at least 2 genes selected from CITED1, TENM1, SCXLY, CCM2 and IGSF10 or the complementary strand thereof, more preferably at least 3, 4 or 5; or for at least 2 distinct domains of an expression product of at least one gene selected from CITED1, TENM1, SCXLY, CCM2 and IGSF10, more preferably at least 3, 4 or 5.

A particular object of the invention relates to a product comprising a support on which are immobilised:
at least 1 probe that specifically hybridizes with an RNA encoded by a CITED1 gene; and
at least 1 probe that specifically hybridizes with an RNA encoded by a TENM1 gene.

A particular object of the invention relates to a product comprising a support on which are immobilised:
at least 1 probe that specifically hybridizes with exon 4 of the sequence of CITED1 gene or of a sequence complementary thereto; and/or
at least 1 probe that specifically hybridizes with exon 3 of the sequence of CITED1 gene or of a sequence complementary thereto, and/or
at least 1 probe that specifically hybridizes with exon (−2) of the sequence of CITED1 gene or of a sequence complementary thereto.

Another particular object of the invention relates to a product comprising a support on which are immobilised:
at least 1 probe that specifically hybridizes with exon1, 3, 13, or 15, of the sequence of TENM1 gene or of a sequence complementary thereto; and
at least 1 probe that specifically hybridizes with exon17, 18, 19, 25, or 32 of the sequence of TENM1 gene or of a sequence complementary thereto.

Another particular object of the invention relates to a product comprising a support on which is immobilised:
from 1 to 5 probes, overlapping or not, each comprising all or part of the sequence of CITED1 gene or of a sequence complementary thereto; and
from 1 to 5 probes, overlapping or not, each comprising all or part of the sequence of TENM1 gene or of a sequence complementary thereto,

wherein, the term “part” is as defined above.

Another particular object of the invention relates to a product comprising a support on which is immobilised:
from 1 to 5 probes, preferably from 1 to 3, typically 2, overlapping or not, each comprising all or part of the sequence of CITED1 gene or of a sequence complementary thereto, preferably all or part of anyone of the sequences listed as SEQ ID NOs: 106-119, or of a sequence complementary thereto;
from 1 to 5 probes, preferably from 1 to 3, typically 2, overlapping or not, each comprising all or part of the sequence of TENM1 gene or of a sequence complementary thereto, preferably all or part of anyone of the sequences listed as SEQ ID NOs: 817-838, or of a sequence complementary thereto;
from 1 to 5 probes, preferably from 1 to 3, typically 2, overlapping or not, each comprising all or part of the sequence of SCLY gene or of a sequence complementary thereto, preferably of SEQ ID NO: 749, or of a sequence complementary thereto;
from 1 to 5 probes, preferably from 1 to 3, typically 2, overlapping or not, each comprising all or part of the sequence of CCM2 gene or of a sequence complementary thereto, preferably of SEQ ID NO: 83, or of a sequence complementary thereto;
from 1 to 5 probes, preferably from 1 to 3, typically 2, overlapping or not, each comprising all or part of the sequence of IGSF10 gene or of a sequence complementary thereto, preferably of SEQ ID NO: 335, or of a sequence complementary thereto; and
from 1 to 5 probes, preferably from 1 to 3, typically 2, overlapping or not, each comprising all or part of the sequence of FRMD3 gene or of a sequence complementary thereto, preferably all or part of anyone of the sequences listed as SEQ ID NOs: 249 to 269, or of a sequence complementary thereto;

wherein the term “part” advantageously indicates a region as defined above.

The invention also relates to a kit comprising a product such as defined previously and reagents for a hybridisation reaction.

The invention further relates to the use of a product or kit defined above for in vitro or ex vivo diagnosis of thyroid cancer in a subject.

Another object of the present application relates to a product comprising a support on which is immobilised at least two ligands that specifically bind a polypeptide coded by at least 2 genes selected from CITED1, TENM1, CCM2, SCLY, IGSF10 and FRMD3.

The support can be any solid or semi-solid support having at least one surface, flat or not (i.e., in two or three dimensions), allowing the immobilisation of nucleic acids or polypeptides. Such supports are for example a slide, bead, membrane, filter, column, plate, etc. They can be made of any compatible material, such as most notably glass, silica, plastic, fibre, metal, polymer, polystyrene, Teflon, etc. The reagents can be immobilised on the surface of the support by known techniques, or, in the case of nucleic acids, synthesised directly in situ on the support. Immobilisation techniques include passive adsorption (Inouye et al., J. Clin. Microbiol. 28 (1990) 1469) and covalent bonding. Techniques are described for example in WO 90/03382 and WO 99/46403. The reagents immobilised on the support can be placed in a predetermined order, to facilitate detection and identification of the complexes formed, and according to a variable and adaptable density.

In one embodiment, the inventive product comprises a multiplicity of synthetic oligonucleotides, of length between 5 and 100 bases, and specific for one or more genes or RNAs such as previously defined.

The products of the invention typically comprise control molecules, which are used to calibrate and/or standardise the results.

Another object of the present application relates to a kit comprising a compartment or container comprising at least one, preferably several, nucleic acids comprising a complementary and/or specific sequence of one or more genes or RNAs such as previously defined and/or one, preferably several ligands of one or more polypeptides such as previously defined. Preferably, the product comprises at least 5 distinct nucleic acids and/or ligands selected from the nucleic acids and ligands mentioned above.

Another object of the invention relates to the use of a product or kit such as defined above for the diagnosis of thyroid cancer in a mammalian subject, preferably a human subject.

It is understood that any equivalent technique can be used within the scope of the present application to determine the presence of a target molecule.

Further aspects and advantages of the present invention will appear upon consideration of the following examples, which must be regarded as illustrative and non-restrictive.

### EXAMPLES

A. Material and Methods

1. Biological Samples:

Candidate signatures have been identified using retrospective cohorts (DeCanThyr, Hospices Civils de Lyon and FNAC, Centre Baclesse—Caen) on a total of 60 subjects, 20 patients had malignant nodules and 40 patients had benign lesions. All samples were previously classified as “indeterminate” according to the Bethesda classification and were associated with the histopathological analysis results of the surgical specimen. Table 1 below provides details about the distribution of cancerous and benign thyroid lesions in each Bethesda indeterminate category.

TABLE 1

| | Bethesda | | Histology | Histology |
|---|---|---|---|---|
| Samples | classification | Cytology based | benign | malignant |
| | III | 12 | 11 | 1 |
| 60 patients | IV | 29 | 22 | 7 |
| | V | 19 | 7 | 12 |

2. Total RNA Extraction or Storage

Dedicated fine needle aspirations were collected in 350 μl of RLT buffer (RNeasy Micro kit, Qiagen) and stored at −80° C. Total RNA was extracted using the manufacturer's recommended procedure and stored at −80° C.

The acceptance criteria were the following:
Absorbance measure (A260/A280) greater then 1.7
Ribosomal ratio (28S/18S) greater or equal to 0.4
RIN value (Agilent Bioanalyzer 2100) greater than 4

3. HsGWSA2.0.2.Dx POC 1M Agilent Array

The Hs GWSA 2.0.2.Dx POC 1M Microarray on the Agilent platform is a tool created to detect and quantify gene expression and alternative expression of transcripts. To achieve this, gene transcripts are identified that represent reference and alternative expression patterns and are processed to resolve the discriminative regions in these expression pattern. The identified alternative event patterns may include: alternative exons, novel splice sites, elimination of splice sites, skipping of normally constitutive exons or some combination thereof.

Analysis was performed on publically available sequence data from a set of human genes identified in the NCBI release 189 and using the NCBI Human genome release 37.3. These sequences were analyzed with a proprietary software system that processed two overlapping datasets: modeling the exon structure of the annotated reference forms of the genes and generating models to identify alternative events identified by these reference forms within the genes.

The reference form analysis for the genes involved identification of expression regions that represented every exon of the reference exemplar sequence data. The alternative event analysis was performed by modeling expression patterns of the identified alternative transcripts and deriving the discriminative expression regions.

After the expression regions were identified a second analysis was performed to identify a minimally redundant expression path through the gene for all selected expression features (reference form expression and included discriminative expression regions). A series of probes were designed from this path: probes that target the expressed exons of the reference forms, junctions to constitutive exons of the reference forms and probes designed against the identified discriminative regions and the junctions (explicit and implicit) indicated by the evidence of the alternative transcripts. This combination of exon and junction probes enables the platform to specifically monitor the expression of the individual components of the alternatively spliced mRNA populations.

19,391 human genes were submitted to the analysis process. 34056 alternative events were identified that matched the selection and scoring criteria of the analysis and selection algorithms. Probes were designed to the sense strand of the sequence as a 24 nucleotide sequence with a 35 nucleotide poly-T linker and the microarrays were produced by Agilent Technologies.

4. RNA Amplification

15 to 50 ng of total RNA were used as matrix for target synthesis using the Ovation Pico WTA System v2 kit (NuGen, San Carlos, Calif.), as recommended by the manufacturer. The quality and quantity of cDNA generated were evaluated using Agilent's 2100 Bioanalyser (Agilent Technologies) and the NanoDrop 1000 Spectrophotometer (Labtech International, UK). 2 μg of cDNA molecules were labeled using Agilent SureTag DNA labeling kit, following manufacturer's recommended protocol. The quality of the Cy3-labelled cDNA is assessed by measuring the yield of cDNA and the specific activity, proportional to the degree of labeling, by measuring the absorbance of the purified product at 260 nm and 550 nm.

5. Hybridization with GWSA Slide

10 μg of cDNA amplified and labeled with Cy3 were used for the hybridization on the HsGWSA2.0.0.Dx POC 1M Agilent array following manufacturer's recommendation, at 55° C. for 17 hours. Non-specific hybridization signals were removed by a 2-step wash of the arrays, following manufacturer's protocol. Hybridization signals were measured using the G5761A SureScan Dx Microarray Scanner System (Agilent Technologies, USA).

6. Data Extraction and Normalization

Intensity signal measurements were extracted with Feature Extraction 11.0.1.1 software (Agilent Technologies). The files (.Tiff) processed with Feature Extraction were quantile normalized using GeneSpring Gx software (Agilent Technologies) prior to statistical analysis. Expression values below 20 were all floored at 20. Probe sets were filtered out if the ratio of the maximum to minimum intensity across the training set was lower or equal to 2.5 or if the difference between the maximum and minimum intensity was lower or equal to 50. 207 043 probe sets from the 448 863 probe sets on the chips remained for further analysis.

7. Statistical Analysis and Signature Identification

The point biserial correlation coefficient was calculated for probe sets between probe set expression and a binary indicator, the value of which was defined by the malignancy status of the patient (0 for cancer free and 1 for patients with Cancer) (van't Veer, et al. 2002). The probe sets with the highest absolute correlation are those most correlated with the occurrence of thyroid cancer. T-tests to evaluate whether point biserial correlation values were significantly different from 0 were used.

To develop a prediction rule to classify samples from new patients with unknown status, we chose the nearest centroid prediction rule (Michiels et al, 2005). This prediction rule classifies new patients according to the correlation between the expression of their molecular classifier probe sets (or genes or events) and the average profiles in the two categories (the average Benign and Malignant profiles) are defined as the vector of the average expression values of the molecular classifier probe sets in benign and malignant samples in the training set, the predicted category being the one with the highest correlation. The molecular classifier probe sets were defined as those for which the p-value of the point biserial correlation coefficient with the benign or malignant binary status variable was below a particular threshold.

Leave-one-out cross-validation (LOOCV) was used to estimate the prediction accuracy of the rule determined on the training set. One sample is left out, and the remaining samples are used to build the prediction rule, which is then used to classify the left-out sample. The entire model-building process was repeated for each leave-one-out training set to provide unbiased estimates of the prediction accuracy of the prediction rule determined on the entire training set: correlation coefficients were recalculated, sorted according to their absolute value, the molecular classifier probe sets defined, and the nearest centroid prediction rule constructed. We predicted the outcome of the sample that was left out in the first place, on the basis of the highest correlation coefficient it had with the average benign and malignant profiles from the remaining other samples. At the end of the leave-one-out cross-validation procedure, we counted in how many cases the predictions were correct and in how many cases the predictions were incorrect to estimate the misclassification rate of the prediction rule determined on the entire training set. The results obtained are listed in the table below. ROC-curves are constructed to determine optimal cut-offs based on the correlation values with the cancer and benign profiles, and area under the ROC curve (AUC) has been calculated for estimating predictive accuracy.

### Classifier Size Reduction

Starting from the list of probe sets (PS) included in the classifier developed using the nearest centroid prediction (NCP) rule and the 25 PS with p-value below 1e⁻⁷, the correlation between each PS expression level was estimated using Pearson correlation coefficients and associated p-values.

Based on these correlations coefficients (Approach C), 3 different selection methods were used in order to reduce the size of the signature:
1. For a given gene: selection of the PS the most correlated with the other PS of a given gene and the PS the least correlated with the other PS of this gene.
2. For a given gene: selection of the PS the least correlated with the other PS of this gene and the PS the most correlated with the other PS of this gene but slightly correlated with the PS of the other genes.
3. For a given gene: selection of the PS the most correlated with the other PS of this gene, the PS the least correlated with the other PS of this gene and the PS the most correlated with the other PS of this gene but slightly correlated with the PS of the other genes.

Two other approaches were used to reduce the size of the classifiers:
by steps of 5 PS from 5 to 25 PS contained in the 25PS classifier (Approach A)
by selecting the PS for which the correlation with all the patient samples is the most significant (Approach B).

B. Results

Expression values below 20 were all floored at 20. Probe sets (PS) were filtered out if the ratio of the maximum to minimum intensity across the training set was lower or equal to 2.5 or if the difference between the maximum and minimum intensity was lower or equal to 50. 207 043 probe sets out of the 448 863 probe sets per chip remained for further analysis.

The probe sets associated to the highest fold changes and the most statistically significant of a difference between malignant and benign nodules were identified and are listed in Table 2.

TABLE 2
Summary of PS Identification

| Cut-off | Number of significant PS | Signature |
|---|---|---|
| 1e⁻² | 4196 | A |
| 1e⁻³ | 918 | B |
| 1e⁻⁴ | 278 | C |
| 1e⁻⁵ | 109 | D |
| 1e⁻⁶ | 51 | E |
| 1e⁻⁷ | 25 | F |
| 1e⁻⁸ | 12 | G |

Molecular signatures have been developed using the p-value thresholds described above. Table 3 summarizes the leave one out cross-validation results. In this table, the signatures error rates, sensitivities and specificities obtained with the nearest centroid prediction rules are denoted as Error Rate (diff), Sensitivity (diff) and Specificity (diff) because based on the difference in the correlation with the Malignant and Benign profiles.

The accuracy of the signatures is measured by the area under the ROC [Receiver Operating Characteristics] curve. The ROC curve is created by plotting the true positive rate (sensitivity) against the false positive rate (1-specificity) at various threshold settings. The point with coordinate (0,1) (also called perfect classification) in the ROC space is associated to a sensitivity of 100% and a specificity of 100%. A test with perfect discrimination has a ROC curve that passes through the upper left corner (100% sensitivity, 100% specificity). Therefore the closer the ROC curve is to the upper left corner, the higher the overall accuracy of the test (Zweig & Campbell, 1993).

The Area Under the ROC Curve (AUC) based on the difference between the correlation values (Malignant and/or Benign profiles) are labeled as AUC (diff), AUC (M) or AUC (B).

TABLE 3
Summary of Leave-One-Out Cross Validation.

| | | Error | Sensi- | Speci- | | | |
|---|---|---|---|---|---|---|---|
| Sig- | Cut- | Rate | tivity | ficity | AUC | AUC | AUC |
| nature | off | (diff) | (diff) | (diff) | (diff) | (B) | (M) |
| A | 1e⁻² | 18.3% | 60.0% | 92.5% | 70.4% | 76.5% | 62.9% |
| B | 1e⁻³ | 13.3% | 70.0% | 95.0% | 81.9% | 87.4% | 69.8% |
| C | 1e⁻⁴ | 13.3% | 75.0% | 92.5% | 81.9% | 86.5% | 78.0% |
| D | 1e⁻⁵ | 13.3% | 75.0% | 92.5% | 83.0% | 86.1% | 79.3% |
| E | 1e⁻⁶ | 16.7% | 70.0% | 90.0% | 81.8% | 79.5% | 77.1% |
| F | 1e⁻⁷ | 18.3% | 65.0% | 90.0% | 86.0% | 67.5% | 85.1% |
| G | 1e⁻⁸ | 23.3% | 60.0% | 85.0% | 77.0% | 52.1% | 77.0% |

### Performance of Signature B (918PS)

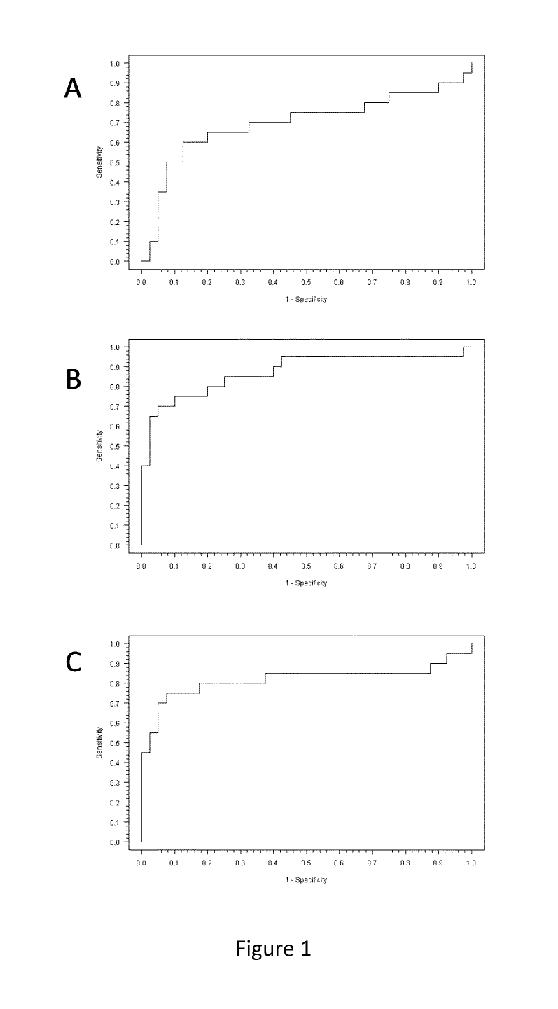

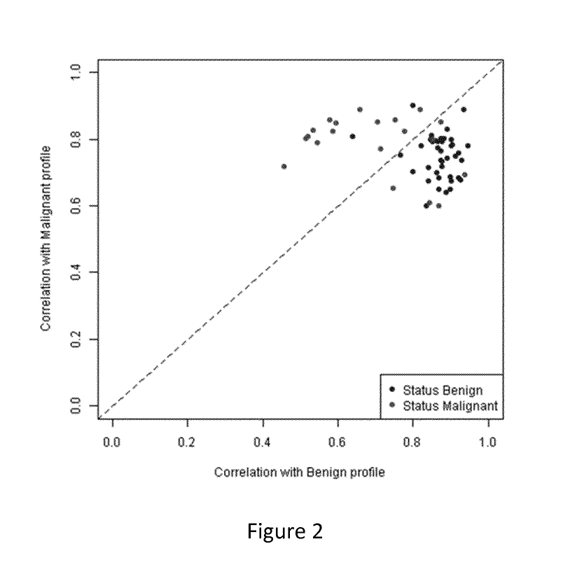

Signature B, based on the combination of 918 probe sets with p-values below 1e⁻³, produces the lowest overall error rate (13.3%). For this signature, the leave-one-out cross validated estimations of sensitivity and specificity are 70% and 95%, respectively. The ROC curves associated to the 918PS signature are presented in FIG. 1. The correlation of each FNA sample with the average malignant and the benign profiles using LOOCV with a p-value cut-off of 10³ is shown in FIG. 2.

Signature B contains a plurality of probes that allows specific detection of at least one expression product of CITED1, TENM1, CCM2, SCLY, IGSF10 and FRMD3 and of each gene listed in Table A and in Table B.

### Performance of Signature D (109PS)

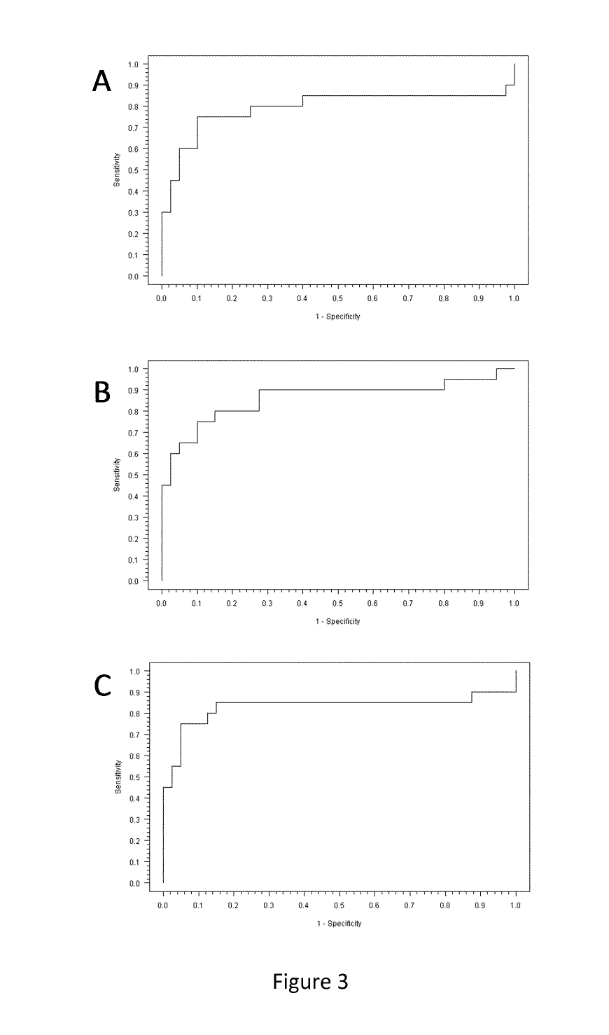

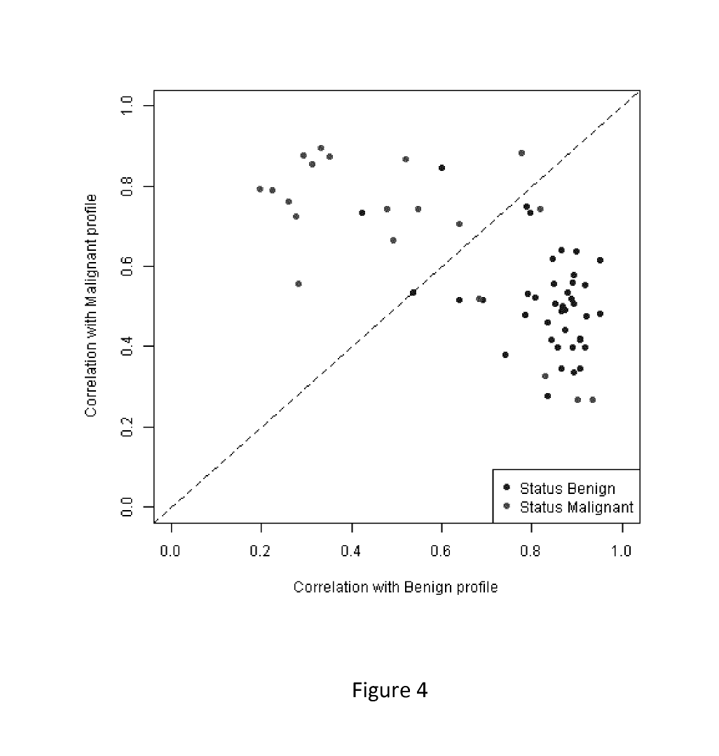

Signature D, based on the combination of 109 probe sets with p-values below 1e⁻⁵, produces also the lowest overall error rate (13.3%). For this signature, the leave-one-out cross validated estimations of sensitivity and specificity are 75% and 92.5%, respectively. The ROC curves associated to the 109PS signature are presented in FIG. 3. The correlation of each FNA sample with the average malignant and the benign profiles using LOOCV with a p-value cut-off of 10⁻⁵ is shown in FIG. 4.

Signature D contains a plurality of probes that allows specific detection of at least one expression product of CITED1, TENM1, CCM2, SCLY, IGSF10 and FRMD3 and of each gene listed in Table A.

### Performance of Signature F (25PS)

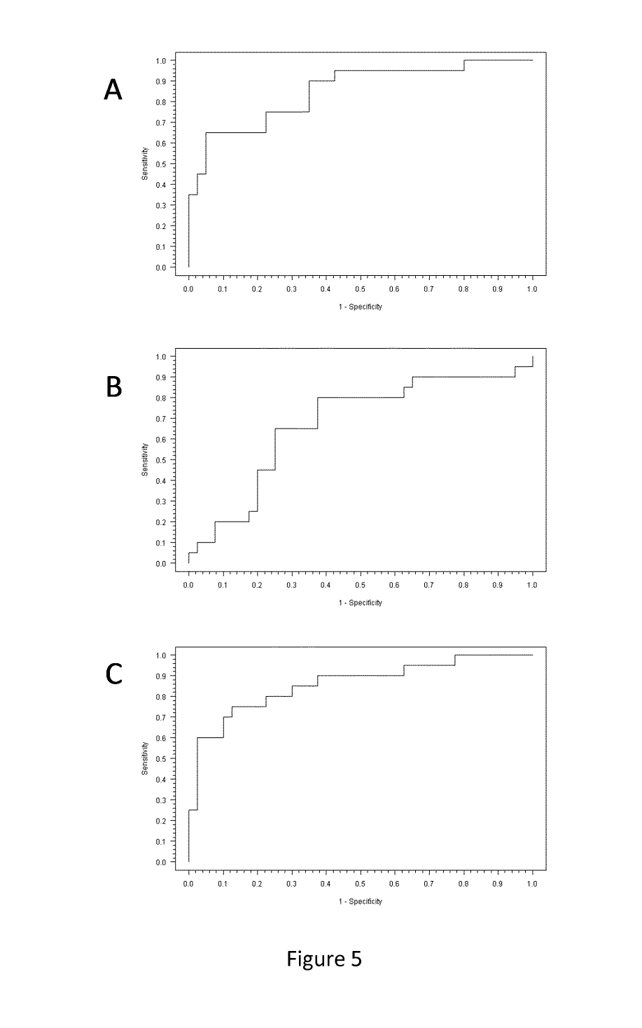

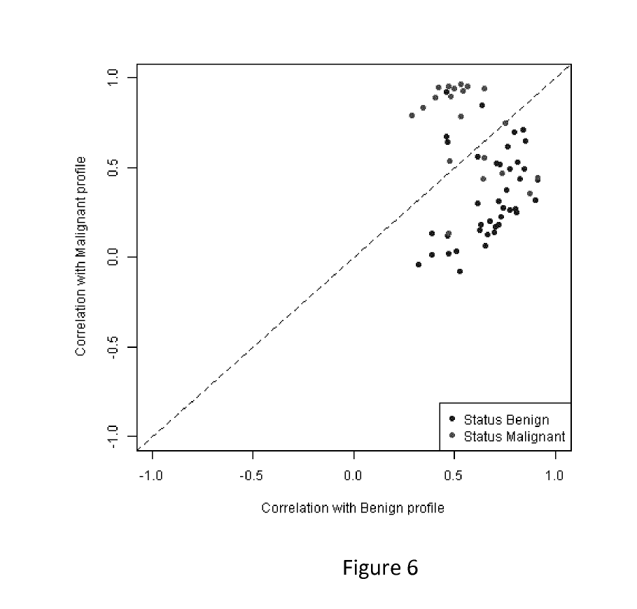

Signature F, based on the combination of 25 probe sets with p-values below 1e⁷, produces an error rate of 18.3%. For this signature, the leave-one-out cross validated estimations of sensitivity and specificity are 65% and 90%, respectively. The ROC curves associated to the 25PS signature are presented in FIG. 5. The correlation of each FNA sample with the average malignant and the benign profiles using LOOCV with a p-value cut-off of 10⁻⁷ is shown in FIG. 6.

Each of these signatures may be used to effectively determine the malignant status of a thyroid lesion with very high specificity and a good sensitivity.

### Analysis of the Content of Signature F (25PS)

In order to identify the smallest signature composition able to classify patient with high accuracy, 3 approaches (see Material and Methods) were selected and applied on the composition of the 25PS signature (90% specificity and 65% sensitivity). The results show that Signature F actually targets 6 distinct marker genes, namely CITED1, TENM1, SCLY, CCM2, IGSF10 and FRMD3. The composition and associated fold changes of the 25 PS are listed in Table 4. These target genes represent valuable markers which can be used to analyse thyroid cancer in patients.

TABLE 4
25 PS signature composition and associated fold changes

| | Fold-Change | | |
|---|---|---|---|
| Column ID | (M/B) | Ranking | Gene |
| HSX2_01477710-333, HSX2_01477710-515\|HSX2_01477710 | 3.52 | 1 | TENM1 |
| HSX2_01477640-162, HSX2_01477640-81\|HSX2_01477640 | 4.88 | 2 | TENM1 |
| HSX2_01477635-192, HSX2_01477635-73\|HSX2_01477635 | 3.57 | 3 | TENM1 |
| HSX2_01477563-23, HSX2_01477563-15\|HSX2_01477563 | 6.53 | 4 | TENM1 |
| HSX2_01477645-87, HSX2_01477645-41\|HSX2_01477645 | 5.67 | 5 | TENM1 |
| HSX2_01477615-1\|HSX2_01477615 | 5.73 | 6 | TENM1 |
| HSX2_01477675-104, HSX2_01477675-288\|HSX2_01477675 | 4.40 | 7 | TENM1 |
| HSX2_01064548-127, HSX2_01064548-32\|HSX2_01064548 | 14.39 | 8 | CITED1 |
| HSX2_01064539-42, HSX2_01064539-77\|HSX2_01064539 | 11.43 | 9 | CITED1 |
| HSX2_02073060-1, HSX2_02073060-141\|HSX2_02073060 | 2.08 | 10 | CCM2 |
| HSX2_02509166-208, HSX2_02509166-401\|HSX2_02509166 | 8.21 | 11 | CITED1 |
| HSX2_01771392-673, HSX2_01771392-460\|HSX2_01771392 | 2.99 | 12 | SCLY |
| HSX2_01064533-104, HSX2_01064533-39\|HSX2_01064533 | 1.85 | 13 | CITED1 |
| HSX2_01064546-23, HSX2_01064546-24\|HSX2_01064546 | 12.74 | 14 | CITED1 |
| HSX2_01477625-72, HSX2_01477625-106\|HSX2_01477625 | 5.46 | 15 | TENM1 |
| HSX2_01064543-27, HSX2_01064543-46\|HSX2_01064543 | 10.95 | 16 | CITED1 |
| HSX2_01477568-13, HSX2_01477568-174\|HSX2_01477568 | 3.14 | 17 | TENM1 |
| HSX2_01477711-4356, HSX2_01477711-469\|HSX2_01477711 | 3.45 | 18 | TENM1 |
| HSX2_01477695-242, HSX2_01477695-17\|HSX2_01477695 | 3.17 | 19 | TENM1 |
| HSX2_02487762-422, HSX2_02487762-163\|HSX2_02487762 | 1.79 | 20 | IGSF10 |
| HSX2_01477665-106, HSX2_01477665-35\|HSX2_01477665 | 3.40 | 21 | TENM1 |
| HSX2_01477573-25, HSX2_01477573-189\|HSX2_01477573 | 3.40 | 22 | TENM1 |
| HSX2_02319619-6, HSX2_02319619-4\|HSX2_02319619 | 5.56 | 23 | FRMD3 |
| HSX2_01477556-78, HSX2_01477556-189\|HSX2_01477556 | 1.76 | 24 | TENM1 |
| HSX2_01064547-31, HSX2_01064547-166\|HSX2_01064547 | 3.44 | 25 | CITED1 |

Starting from the list of probe sets (PS) included in the 25PS signature, the correlation between each PS expression level was estimated using Pearson correlation coefficients and associated p-values. Based on these correlation coefficients, 3 different selection methods (Approach C methods 1, 2 and 3 as described in Material and Methods) as well as the following two approaches were used to reduce the size of the signatures:
by steps of 5 PS from 5 to 25 PS contained in the 25PS signature (Approach A)
by selecting the PS for which the correlation with all the patient samples is the most significant (Approach B)

Signatures associated to error rates below or equal to 20% were identified for the 3 approaches used and are listed in Table 5.

TABLE 5
optimal signatures identified during signature size reduction analysis.

| # PS | Sensitivity | Specificity | Target Genes |
|---|---|---|---|
| 15 | 65% | 87.50% | TENM1, CITED1, CCM2, SCLY |
| 8 | 75% | 82.50% | TENM1, CITED1, CCM2, SCLY, |
| | | | FRMD3, IGSF10 |
| 8 | 70% | 87.50% | TENM1, CITED1, CCM2, SCLY, |
| | | | FRMD3, IGSF10 |
| 10 | 70% | 90.00% | TENM1, CITED1, CCM2, SCLY, |
| | | | FRMD3, IGSF10 |
| 4 | 65% | 92.50% | TENM1, CITED1 |

### PS Analysis Focusing on CITED1 and TENM1

Various probe sets are designed that target particular regions of the CITED1 or TENM1 gene products. Tables 6 and 7 identify the exons targeted by these probes for TENM1 and CITED1, respectively, and the upregulations of these exons measured in the malignant samples of cytological indeterminate thyroid lesions.

TABLE 6
Upregulation and exons identified with the PS composing
the 25 PS signature and covering TENM1 gene.

| | | Fold-Change |
|---|---|---|
| ProbeSet ID | Targeted exon | (M vs. B) |
| HSX2_01477556 | 1 | 1.76 |
| HSX2_01477568 | 4 | 3.14 |
| HSX2_01477695 | 29 | 3.17 |
| HSX2_01477573 | 5 | 3.40 |
| HSX2_01477665 | 23 | 3.40 |
| HSX2_01477711 | UTR | 3.45 |
| HSX2_01477710 | 32 | 3.52 |
| HSX2_01477635 | 17 | 3.57 |
| HSX2_01477675 | 25 | 4.40 |
| HSX2_01477640 | 18 | 4.88 |
| HSX2_01477625 | 15 | 5.46 |
| HSX2_01477645 | 19 | 5.67 |
| HSX2_01477615 | 13 | 5.73 |
| HSX2_01477563 | 3 | 6.53 |

TABLE 7
Upregulation and exons identified with the PS composing
the 25 PS signature and covering CITED1 gene.

| | | Fold-Change |
|---|---|---|
| ProbeSetID | Targeted exon | (M vs. B) |
| HSX2_01064533 | (−2) | 1.85 |
| HSX2_01064547 | 4 | 3.44 |
| HSX2_02509166 | 4 | 8.21 |
| HSX2_01064543 | 4 | 10.95 |
| HSX2_01064539 | 3 | 11.43 |
| HSX2_01064546 | 4 | 12.74 |
| HSX2_01064548 | 4 | 14.39 |

Various combinations of such probes were assessed for relevance. The invention shows that when a limited number of target genes are monitored according to the invention, it is highly preferred to use two distinct probes specific for distinct regions of the expression product of said gene. In this regard, monitoring exon4 and/or exon3 and/or exon-2 of CITED1 and targeting exons 32, 25, 19, 18, 17, and/or 1, for TENM1 is particularly advantageous.

The combination of the following particular exons of CITED1 provides strong sensitivity:

For_CITED1 gene, the targeting of exon3, exon4 and exon-2 with the following probe sets increases performance:

### ProbeSets:

### HSX2_01064548

### HSX2_01064539

### HSX2_02509166

### HSX2_01064533

Sensitivity 73% and Specificity 83%

### ProbeSets:

### HSX2_01064548

### HSX2_01064539

### HSX2_02509166

### HSX2_01064533

### HSX2_01064546

### HSX2_01064543

### HSX2_01064547

Sensitivity 75% and Specificity 90%

These results show that an effective and reliable detection of thyroid cancer can be implemented by monitoring 2 or 3 distinct domains of CITED1 expression product.

As discussed below, by further combining such marker with TENM1, a further specific and sensitive detection is obtained.

### Signature H: CITED1 & TENM1

In this experiment, two probeSets specific for CITED1 and two probeSets specific for TENM1 were combined. The specific probeSets used are listed below.

### TENM1:

### HSX2_01477675

### HSX2_01477556

### CITED1:

### HSX2_01064533

### HSX2_01064546

Sensitivity 65% and Specificity 92.5%

Further exon analysis results linked to the performances listed in table 5 are reported in Table 8, focusing only on CITED1 and TENM1.

TABLE 8
targeted exons for TENM1 and CITED1 in optimal signatures

| | | Targeted | | |
|---|---|---|---|---|
| | | exons | | |
| # PS | Targeted exons TENM1 | CITED1 | Sensitivity | Specificity |
| 15 | 32, 18, 17, 3, 19, 13, 25, 15 | 3, 4, (−2) | 65% | 87.50% |
| 8 | 32, 18 | 3, 4 | 75% | 82.50% |
| 8 | 32, 18, 17 | 3, 4, (−2) | 65% | 87.50% |
| 8 | 25, 1 | 4, (−2) | 70% | 87.50% |
| 10 | 25, 19, 1 | 4, (−2) | 70% | 90.00% |
| 4 | 1, 25 | 4, (−2) | 65% | 92.50% |

These results show that by detecting exon1, 19 and/or 25 of TENM1 and exon 4 and/or (−2) of CITED1, highly relevant diagnosis can be made.

### REFERENCES

Curado, M. P., B. Edwards, et al. (2007). “Cancer Incidence in Five Continents.” IARC Scientific Publications 9(160): 1-10.
Cibas, E. S. and S. Z. Ali (2009). “The Bethesda System for Reporting Thyroid Cytopathology.” Thyroid 19(11): 1159-1165.
Nikiforov, Y. E., N. P. Ohori, et al. (2011). “Impact of mutational testing on the diagnosis and management of patients with cytologically indeterminate thyroid nodules: a prospective analysis of 1056 FNA samples.” J Clin Endocrinol Metab 96(11): 3390-3397.
Alexander E K, Kennedy G C, Baloch Z W, Cibas E S, Chudova D, Diggans J, Friedman L, Kloos R T, LiVolsi V A, Mandel S J, Raab S S, Rosai J, Steward D L, Walsh P S, Wilde J I, Zeiger M A, Lanman R B, Haugen B R. Preoperative diagnosis of benign thyroid nodules with indeterminate cytology. N Engl J Med. 2012 Aug. 23; 367(8):705-15. doi: 10.1056/NEJMoa1203208. Epub 2012 Jun. 25
Hu S, Ewertz M, Tufano R P, Brait M, Carvalho A L, Liu D, Tufaro A P, Basaria S, Cooper D S, Sidransky D, Ladenson P W, Xing M. Detection of serum deoxyribonucleic acid methylation markers: a novel diagnostic tool for thyroid cancer. J Clin Endocrinol Metab. 2006 January; 91(1):98-104. Epub 2005 Nov. 1.
Nikiforova M N, Wald A I, Roy S, Durso M B, Nikiforov Y E. Targeted next-generation sequencing panel (ThyroSeq) for detection of mutations in thyroid cancer. J Clin Endocrinol Metab. 2013 November; 98(11):E1852-60.
Zweig M H1, Campbell G. Receiver-operating characteristic (ROC) plots: a fundamental evaluation tool in clinical medicine. Clin Chem. 1993 April; 39(4):561-77.
Van't Veer, L. J., H. Dai, et al. (2002). “Gene expression profiling predicts clinical outcome of breast cancer.” Nature 415(6871): 530-536.
Michiels S, Koscielny S, Hill C. Prediction of cancer outcome with microarrays: a multiple random validation strategy. Lancet 2005; 365:488-92.

| Entrez | Entrez | | SEQ | |
|---|---|---|---|---|
| ID | Name | ProbeSet ID | ID # | Target Sequence |
| 51115 | RMDN1 | HSX2_01747164 | 1 | GCCTTAGGCTACTTTCACAGGGCAGAACAAG |
| 285533 | RNF175 | HSX2_02344199 | 2 | CTTTCATGAATTCTGCATCCGAGGTTGGTGTATCGTTGGGAAAAAGCAGACTTGC |
| | | | | CCTTACTGCAA AGAGAAAGTTGATTTGAAGAGGATGATCAGTAATCC |
| 10921 | RNPS1 | HSX2_02538806 | 3 | GGCGGCGGCGGGAAGATGATGCTGCTCTTTGGCG |
| 79897 | RPP21 | HSX2_02030256 | 4 | ATTCCAAACCACTACAACCCTTGCCAAACACAGCCCACTCCATTTCAGACCGCCTT |
| | | | | CCTGAGGAGA |
| | | | | AAATGCAGACTCAGGGTTCCAGTAACCAGTGATGGATTCACCCCATCTCCCAAAT |
| | | | | AAAGTTTACTT GTTTTACATTC |
| 10351 | ABCA8 | HSX2_01491300 | 5 | GCTATTAATTCTAATGGCTGGATTTTGCCCTCTTCTTGTGGAGTATACCATGGTGA |
| | | | | AAATATATCA |
| | | | | AAACAGTTACACCTGGGAACTTTCTCCTCATTTGTATTTCCTTGCTCCTGGACAAC |
| | | | | AACCACATGAC CCTCTCACTCAACTACTGATCATCAATAAAACAG |
| 8714 | ABCC3 | HSX2_01355653 | 6 | GTTCTGGCCCCTGTCATCCTCATGCTGCTCAATTCCTTCTTCAACGCCATCTCCACT |
| | | | | CTTGTGGTCA |
| | | | | TCATGGCCAGCACGCCGCTCTTCACTGTGGTCATCCTGCCCCTGGCTGTGCTCTAC |
| | | | | ACCTTAGTGC AG |
| 35 | ACADS | HSX2_00754990 | 7 | GCCATCCAGATCCTGGGCGGCATGGGCTACGTGACAGAGATGCCGGCAGAGCG |
| | | | | GCACTACCGC |
| | | | | GACGCCCGCATCACTGAGATCTACGAGGGCACCAGCGAAATCCAGCGGCTGGTG |
| | | | | ATCGCCGGGC |
| | | | | ATCTGCTCAGGAGCTACCGGAGCTGAGCCCGCGGCGGACTGCCCCAGGACTGCG |
| | | | | GGAAGGCGC |
| | | | | GGGAGCCAGGGGCCTCCACCCCAACCCCGGCTCAGAGACTGGGCGGCCCGGCG |
| | | | | GGGGCTCCCT |
| | | | | GGGGACCCCAGATGGGCTCAGTGCTGCCACCCAGATCAGATCACATGGGAATGA |
| | | | | GGCCCTCCGA |
| | | | | CCATTGGCAGCTCCGCCTCTGGGCCTTTCCGCCTCCTCACCACTGTGCCTCAAGTT |
| | | | | CCTCATCTAA |
| | | | | GTGGCCCTGGCCTCCTGGGGGCGGGGTTGTGGGGGGGCTGAGCGACACTCAGG |
| | | | | GACACCTCAG |
| | | | | TTGTCCTCCCGCGGGCCCTGGTGCCCTGGCATGAAGGCCCAGTGCGACAGGCCC |
| | | | | TTGGTGGGGT |
| | | | | CTGTCTTTTCCTTGAGGTCAGAGGTCAGGAGCAGGGCTGGGGTCAGGATGACGA |
| | | | | GGCCTGGGGT |
| | | | | CCTGGTGTTGGGCAGGTGGTGGGGCTGGGCCATGGAGCTGGCCCAGAGGCCCC |
| | | | | TCAGCCCTTTG |
| | | | | TAAAGTCTGATGAAGGCAGGGGTGGTGATTCATGCTGTGTGACTGACTGTGGGT |
| | | | | AATAAACACA CCTGTCCCCCA |
| 79968 | WDR76 | HSX2_02034207 | 8 | GTAAATGAATATAAAGAAAATCAAAACATCGCTTATGTGTCTCTGAGACCAGCAC |
| | | | | AGACTACAGT TTTAATAAAAACAGCTAAGGTCTATCTTGCCCCCTTTTCACTCAG |
| 835 | CASP2 | HSX2_00819195 | 9 | GCCACCTGGAGGATATGTTGCTCACCACCCTTTCTGGGCTTCAGCATGTACTCCCA |
| | | | | CCG |
| 56999 | ADAMTS9 | HSX2_01905394 | 10 | GGAGGGAGGGGTGGGAAGCACCATGCAGTTTGTATCCTGGGCCACACTGCTAAC |
| | | | | GCTCCTGGTG |
| | | | | CGGGACCTGGCCGAGATGGGGAGCCCAGACGCCGCGGCGGCCGTGCGCAAGG |
| | | | | ACAGGCTGCA CCCGAGGCAAG |
| 56999 | ADAMTS9 | HSX2_01905410 | 11 | CTGGGCACATTCCGGTCTCATGATGGGGATTATTTTATTGAACCACTACAGTCTAT |
| | | | | GGATGAACAA |
| | | | | GAAGATGAAGAGGAACAAAACAAACCCCACATCATTTATAGGCGCAGCGCCCCC |
| | | | | CAGAGAGAGC CCTCAACAGGAAGGCATGCATGTGACACCTCAG |
| 56999 | ADAMTS9 | HSX2_01905487 | 12 | GTTTAACATGCCTCATGATGACAACAACAAATGTAAAGAAGAAGGAGTTAAGAG |
| | | | | TCCCCAGCATG |
| | | | | TCATGGCTCCAACACTGAACTTCTACACCAACCCCTGGATGTGGTCAAAGTGTAG |
| | | | | TCGAAAATAT ATCACTGAGTTTTTAGA |
| 56999 | ADAMTS9 | HSX2_01905592 | 13 | TGTTCAAAAAGCTGTGACGGTGGGACCCAGAGGAGAAGGGCTATTTGTGTCAAT |
| | | | | ACCCGAAATG |
| | | | | ATGTACTGGATGACAGCAAATGCACACATCAAGAGAAAGTTACCATTCAGAGGT |
| | | | | GCAGTGAGTTC CCTTGTCCACAGTGGAAATCTGGAGACTGGTCAGAG |
| 56999 | ADAMTS9 | HSX2_01905598 | 14 | TGCTTGGTCACCTGTGGAAAAGGGCATAAGCACCGCCAGGTCTGGTGTCAGTTT |
| | | | | GGTGAAGATC |
| | | | | GATTAAATGATAGAATGTGTGACCCTGAGACCAAGCCAACATCTATGCAGACTTG |
| | | | | TCAGCAGCCG GAATGTGCATCCTGGCAGGCGGGTCCCTGGGGACAG |
| 56999 | ADAMTS9 | HSX2_01905636 | 15 | TGCTCTGTGACCTGTGGGCAAGGTAGGGCAACCCGGCAAGTGATGTGTGTCAAC |
| | | | | TACAGTGACC |
| | | | | ACGTGATCGATCGGAGTGAGTGTGACCAGGATTATATCCCAGAAACTGACCAGG |
| | | | | ACTGTTCCATG |
| | | | | TCACCATGCCCTCAAAGGACCCCAGACAGTGGCTTAGCTCAGCACCCCTTCCAAA |
| | | | | ATGAGGACTA |
| | | | | TCGTCCCCGGAGCGCCAGCCCCAGCCGCACCCATGTGCTCGGTGGAAACCAGTG |
| | | | | GAGAACTGGC CCCTGGGGAGCA |
| 56999 | ADAMTS9 | HSX2_01905650 | 16 | TGCACTAAGCTGTGTGGTGGAGGCATAAGAACAAGACTGGTGGTCTGTCAGCGG |
| | | | | TCCAACGGTG |
| | | | | AACGGTTTCCAGATTTGAGCTGTGAAATTCTTGATAAACCTCCCGATCGTGAGCA |
| | | | | GTGTAACACA |
| | | | | CATGCTTGTCCACACGACGCTGCATGGAGTACTGGCCCTTGGAGC |
| 56999 | ADAMTS9 | HSX2_01905665 | 17 | TGCTCTGTGTCCTGTGGCCGAGGCGTACAGCAGAGGCATGTGGGCTGTCAGATC |
| | | | | GGAACACACA |
| | | | | AAATAGCCAGAGAGACCGAGTGCAACCCATACACCAGACCGGAGTCGGAACGC |
| | | | | GACTGCCAAG |
| | | | | GCCCACGGTGTCCCCTCTACACTTGGAGGGCAGAGGAATGGCAAGAA |
| 56999 | ADAMTS9 | HSX2_01905746 | 18 | GATGGTACCCGAGTCGTAGGGAAATGCGGTGGTTACTGTGGAAAATGCACTCCA |
| | | | | TCCTCTGGTAC TGGCCTGGAGGTGCGAGTTTTATAGCTAAG |
| 56999 | ADAMTS9 | HSX2_01905758 | 19 | AGACCAAAGAGATTAGGAAAAGCCTGGCAGTATTCTCCAACTCCAAACAAGCTCT |
| | | | | AGAGTGCTCC |
| | | | | AGGAAAAGTTATATTCAGTATATGAATAAGTGTTATTCTCCATTATTAATGTGTTC |
| | | | | TGAAAATATAT TATGAATAAATACATCACCACACCCAA |
| 56999 | ADAMTS9 | HSX2_02560668 | 20 | TGCACTAAGCTGTGTGGTGGAGGCATAAGAACAAGACTGGTGGTCTGTCAGCGG |
| | | | | TCCAACGGTG |
| | | | | AACGGTTTCCAGATTTGAGCTGTGAAATTCTTGATAAACCTCCCGATCGTGAGCA |
| | | | | GTGTAACACA |
| | | | | CATGCTTGTCCACACGACGCTGCATGGAGTACTGGCCCTTGGAGCTCG |
| 132 | ADK | HSX2_00763371 | 21 | CTTACAGTTTCCCCAGAGTCAGTATTAAAGGTGGATGTTGATATATGAAAGTTAT |
| | | | | GCCTTCTCACC |
| | | | | ATGCTTCTGAAAACAACAGGATTTTCACTTTGAATCTATCTGCACCGTTTATTAGC |
| | | | | CAGTTCTACAA GGAATCATTG |
| 132 | ADK | HSX2_00763381 | 22 | GAAGCTGCCACTTTTGCTAGAGAGCAAGGCTTTGAG |
| 132 | ADK | HSX2_00763387 | 23 | CATTAAAGAGATAGCCAAAAAGACACAAGCCCTGCCAAAGATGAACTCAAAGAG |
| | | | | GCAGCGAATC GTGATCTTCACCCAAGGGAGAGATGACACTATAATGGCT |
| 132 | ADK | HSX2_02491081 | 24 | AGAAAATATTCTCTTTGGAATGGGAAATCCTCTGCTTGACATCTCTGCTGTAGTG |
| | | | | GACAAAGATTT CCTTGATAA |
| 132 | ADK | HSX2_02491083 | 25 | GGCTTTTTTCTTACAGTTTCCCCAGAGTCAGTATTAAAGGTGGCTCACCATGCTTC |
| | | | | TGAAAACAAC |
| | | | | AGGATTTTCACTTTGAATCTATCTGCACCGTTTATTAGCCAGTTCTACAAGGAATC |
| | | | | ATTGATGAAA GTTATGCCTTATGTTGATATACTTTTTGGAAATGAGACA |
| 132 | ADK | HSX2_02491084 | 26 | ACTAAAGACATTAAAGAGATAGCCAAAAAGACACAAGCCCTGCCAAAGATGAAC |
| | | | | TCAAAGAGGC |
| | | | | AGCGAATCGTGATCTTCACCCAAGGGAGAGATGACACTATAATGGCTACAG |
| 134 | ADORA1 | HSX2_00763516 | 27 | CTTTCCAGGCCGCCTACATCGGCATCGAGGTGCTCATCGCCCTGGTCTCTGTGCC |
| | | | | CGGGAACGTG |
| | | | | CTGGTGATCTGGGCGGTGAAGGTGAACCAGGCGCTGCGGGATGCCACCTTCTG |
| | | | | CTTCATCGTGT |
| | | | | CGCTGGCGGTGGCTGATGTGGCCGTGGGTGCCCTGGTCATCCCCCTCGCC |
| 165 | AEBP1 | HSX2_02491233 | 28 | AGTGTCCCCCCATTGGGATGGAGTCACACCGTATTGAGGACAACCAGATCCGAG |
| | | | | CCTCCTCCATG |
| | | | | CTGCGCCACGGCCTGGGGGCACAGCGCGGCCGGCTCAACATGCAG |
| 10551 | AGR2 | HSX2_01509381 | 29 | TGGAGAAAATTCCAGTGTCAGCATTCTTGCTCCTTGTGGCCCTCTCCTACACTCTG |
| | | | | GCCAGAGATA |
| | | | | CCACAGTCAAACCTGGAGCCAAAAAGGACACAAAGGACTCTCGACCCAAACTGC |
| | | | | CCCAGACCCT CTCCA |
| 126133 | ALDH16A1 | HSX2_02196227 | 30 | GTGTTCCAGGCTGGTGATGTGCCTTCGGAACGCCCATTCTATCCCCCAACCTTGG |
| | | | | TCTCCAACCTGCCCCCAGCCTCCCCATGTGCCCAGGTGGAG |
| 23541 | SEC14L2 | HSX2_02544485 | 31 | ATGTCCTGCGGTTTGACAACACCTACAGCTTCATTCAT |
| 51479 | ANKFY1 | HSX2_01767855 | 32 | CTTCTTGCGGGAGCCAAAGTGAACGAATTAACCAAGCATCGCCAGACTGCCCTC |
| | | | | CATCTTGCTGC |
| | | | | CCAGCAGGACCTGCCCACCATCTGCTCAGTCCTCCTAGAGAATGGCGTGGACTTT |
| | | | | GCTGCCGTGG ATGAGAATGGAAACAATG |
| 60314 | C12ORF10 | HSX2_02562966 | 33 | ATGTATGAGAACTTTGTGGAGGAGGTGGATGCTGTGGACAATGGGATCTCCCAG |
| | | | | TGGGCAGAG |
| | | | | GGGGAGCCTCGATATGCACTGACCACTACCCTGAGTGCACGAGTTGCTCGACTT |
| | | | | AATCCTACCTG GAACCACCCCGACCAAGACACTGAG |
| 6415 | SEPW1 | HSX2_01213456 | 34 | TTTGAAGTGATGGTAGCCGGGAAGTTGATTCACTCTAAG |
| 6415 | SEPW1 | HSX2_02518360 | 35 | TGGCGCTTGAGGCTACAAGTCCAAG |
| 55129 | ANO10 | HSX2_01833062 | 36 | GCTGCAAGCTGCAAGCTGGTCTCTCTTCCACGGTATTCATGGAGCACCAACTCAG |
| | | | | TGCCAGGCAC |
| | | | | TGTCATAGGTCCTGGGGTCTAGTCATGGCCCTGCTCTCTGGAGACCACATACCAG |
| 334 | APLP2 | HSX2_00781384 | 37 | GTAAGAATAAAGTGGATGAAAACATG |
| 143872 | ARHGAP42 | HSX2_02226497 | 38 | GTGACTCCTATAGCAGCAGCCCAGACAGCACACCTATGGGGAGCATTGAGTCAC |
| | | | | TCTCTTCTCAT |
| | | | | TCCTCTGAACAAAATAGCACTACAAAGTCAGCTTCCTGCCAGCCCAGGGAGAAA |
| | | | | TCTGGAGGGAT |
| | | | | TCCTTGGATTGCAACCCCATCATCTTCCAATGGACAGAAAAGCCTTGGTCTGTGG |
| | | | | ACAACTAGTCC |
| | | | | TGAATCAAGTTCCAGAGAAGATGCAACCAAGACAGATGCAGAATCAGACTGCCA |
| | | | | GAGTGTTGCT |
| | | | | TCAGTCACTAGCCCAGGAGACGTTTCCCCACCCATAGACCTAGTCAAGAAAGAG |
| | | | | CCTTATGGGCT |
| | | | | TTCAGGACTGAAAAGAGCTTCTGCTTCTTCTCTCAGATCCATCTCTGCAGCTGAA |
| | | | | G |
| 143872 | ARHGAP42 | HSX2_02226509 | 39 | GAAACAAGAGCTACAGTGGATCTATTCAAAGCTTAACTTCTGTAGGTTCCAAGG |
| | | | | AGACACCCAAA |
| | | | | GCTTCACCAAACCCAGACCTGCCTCCGAAAATGTGCAGGAGATTAAGACTAGAC |
| | | | | ACTGCCTCAAG CAATGGCTATCAGCGGCCTGGCTCAGT |
| 7024 | TFCP2 | HSX2_01262294 | 40 | AGATGATGTGATCCAAATCTGTGGCCCTGCAGATGGAATCAGACTTTTTAATGCA |
| | | | | TTAAAAGGCC G |
| 7024 | TFCP2 | HSX2_02521603 | 41 | GGGCAGATTTATTGAAATTAACTAGAGATGATGTGATCCAAATCTGTGGCCCTGC |
| | | | | AGATGGAATC AGACTTTTTAATGCATTAAAAGGCCG |
| 51566 | ARMCX3 | HSX2_01772667 | 42 | TGGGTCCAAGCCTCTCCCGGGTGGC |
| 51566 | ARMCX3 | HSX2_02552621 | 43 | TGGGTCCAAGCCTCTCCCGGGTGGCCAG |
| 54470 | ARMCX6 | HSX2_02553938 | 44 | GTTTGTAGGTCTGTTGGCCTGTAGGTTTCGGCACAAGTTTCAGCGAGAGAAGGA |
| | | | | GAAAACTGCCT TGGTTGGAACCTTGCAG |
| 430 | ASCL2 | HSX2_00787901 | 45 | CCTCAGCCCCGGAAGCCGAGCGAGCGGCCGGCGCGCTCATCGCCGGGGAGCCC |
| | | | | GCCAGGTGGA |
| | | | | CCGGCCCGCGCTCCGCCCCCAGCGAGCCGGGGACCCACCCACCACCCCCCGCAC |
| | | | | CGCCGACGCC |
| | | | | GCCTCGTTCGTCCGGCCCAGCCTGACCAATGCCGCGGTGGAAACGGGCTTGGAG |
| | | | | CTGGCCCCAT |
| | | | | AAGGGCTGGCGGCTTCCTCCGACGCCGCCCCTCCCCACAGCTTCTCGACTGCAGT |
| | | | | GGGGCGGGG |
| | | | | GGCACCAACACTTGGAGATTTTTCCGGAGGGGAGAGGATTTTCTAAGGGCACAG |
| | | | | AGAATCCATTT TCTACACATTAACTTGAGCTGCTGGAGGGACAC |
| 23250 | ATP11A | HSX2_01615000 | 46 | GTGTGGCTATTTACACGGGAATGGAAACCAAGATGGCATTAAATTATCAATCAA |
| | | | | AATCTCAGAAG CGATCTGCCGTGGAAAA |
| 23250 | ATP11A | HSX2_01615045 | 47 | TTCCTCAAGGCATTCACGGACTTCCTGGCCTTCATGGTCCTCTTTAACTACATCAT |
| | | | | CCCTGTGTCCA |
| | | | | TGTACGTCACGGTCGAGATGCAGAAGTTCCTCGGCTCTTACTTCATCACCTGGGA |
| | | | | CGAAGACATG |
| | | | | TTTGACGAGGAGACTGGCGAGGGGCCTCTGGTGAACACGTCGGACCTCAATGA |
| | | | | AGAGCTGGGA CAG |
| 84239 | ATP13A4 | HSX2_02091652 | 48 | CTGGAGTTCAAGAGCTGGAATCACGCGTCCTGGTGCCTGGAGATTTATTAATTTT |
| | | | | GACAGGGAAC |
| | | | | AAAGTGCTAATGCCATGTGATGCCGTTCTGATTGAAGGCAGCTGTGTGGTGGAT |
| | | | | GAAGGCATGC TGACAG |
| 84239 | ATP13A4 | HSX2_02091695 | 49 | GAAATGGCTTTTTCTGGGGACGATTTCCACATCAAGGGAGTGCCGGCACATGCC |
| | | | | ATGGTAGTTAA GCCCTGCAGAACAGCCAGCCAG |
| 51310 | SLC22A17 | HSX2_02551672 | 50 | CTTCATTGCCCATGCCATTCGCCACTGCTACCAGCCTGTGGGAGGAGGAGGGAG |
| | | | | CCCATCGGACT |
| | | | | TCTACCTGTGCTCTCTGCTGGCCAGCGGCACCGCAGCCCTGGCCTGTGTCTTCCT |
| | | | | GGGGGTCACC |
| | | | | GTGGACCGATTTGGCCGCCGGGGCATCCTTCTTCTCTCCATGACCCTTACCGGCA |
| | | | | TTGCTTCCCTG GTCCTGCTGGGCCTGTGGGATT |
| 27241 | BBS9 | HSX2_02548207 | 51 | GTGGTTGTACTACAATCCCAGAGTCAGACCTAGAAGAAAGATCAGTAGAACAAG |
| | | | | ACTCTACAGA |
| | | | | ACTGTTTACCAACCACAGACATCTCACTGCAGAGACACCCAGGCCTG |
| 607 | BCL9 | HSX2_00799653 | 52 | TAGCCCTAAGTCAAAGCAGGAGGTGATGGTCCGTCCCCCTACAGTGATGTCCCCA |
| | | | | TCTGGAAACC CCCAGCTGGATTCCAAATTCTCCAATCAGG |
| 607 | BCL9 | HSX2_00799660 | 53 | AATGTAATTCTGCTGACCACATAAAGTCCCAGGATTCCCAGCACACACCACACTC |
| | | | | GATGACCCCAT |
| | | | | CAAATGCTACAGCCCCCAGGTCTTCTACCCCCTCCCATGGCCAAACTACTGCCACA |
| | | | | GAGCCCACAC |
| | | | | CTGCTCAGAAGACTCCAGCCAAAGTGGTGTACGTGTTTTCTACTGAGATGGCCAA |
| | | | | TAA |
| 607 | BCL9 | HSX2_02493341 | 54 | TAGCCCTAAGTCAAAGCAGGAGGTGATGGTCCGTCCCCCTACAGTGATGTCCCCA |
| | | | | TCTGGAAACC |
| | | | | CCCAGCTGGATTCCAAATTCTCCAATCAGGGTAAACAGGGGGGCTCAGCCAGCC |
| | | | | AATCCCAGCCA |
| | | | | TCCCCCTGTGACTCCAAGAGTGGGGGCCATACCCCTAAAGCACTCCCTGGCCCAG |
| | | | | GTGGGAGCA |
| | | | | TGGGGCTGAAGAATGGGGCTGGAAATGGTGCCAAGGGCAAGGGGAAAAGGGA |
| | | | | GCGAAGTATT |
| | | | | TCCGCCGACTCCTTTGATCAGAGAGATCCTGGGACTCCAAACGATGACTCTGACA |
| | | | | TTAAAG |
| 6585 | SLITT | HSX2_01227688 | 55 | AATGCTGCGGAACAACCGCATCAGCTGCATCCACAACGACAGCTTCACGGGCCT |
| | | | | GCGCAACGTC |
| | | | | CGGCTCCTCTCGCTCTACGACAACCAGATCACCACCGTATCCCCAGGAGCCTTCG |
| | | | | ACACCCTCCA GTCCCTCTCCACACT |
| 6585 | SLITT | HSX2_01227758 | 56 | GTCTCCACGGCAGAGGACAATGGGATCCTTCTGTACAACGGGGACAACGACCAC |
| | | | | ATTGCAGTTG |
| | | | | AGCTGTACCAGGGCCATGTGCGTGTCAGCTACGACCCAGGCAGCTACCCCAGCT |
| | | | | CTGCCATCTAC AG |
| 627 | BDNF | HSX2_00800274 | 57 | CCATTCACCGCGGAGAGGGCTGCTCTCGCTGCCG |
| 79365 | BHLHE41 | HSX2_02006257 | 58 | ACTCTGGGACATCTGGAGAAAGCTGTAGTCTTGGAATTAACTTTGAAACACTTAA |
| | | | | AAGCTTTAACC |
| | | | | GCCTTAACCGAGCAACAGCATCAGAAGATAATTGCTTTACAGAATG |
| 440335 | SMIM22 | HSX2_02404970 | 59 | GAAAGACCCAAGGGAGTGGATAACTTGGCCCTGGAACCCTGACCCTGTGTCTCC |
| | | | | TGCCCGGTGG CAGTAACAAAGCCTTCTGTCTGCCCAGA |
| 89927 | C160RF45 | HSX2_02130811 | 60 | AAAAAGAAAAAACCAAACTGCAGAAGCAGAGAGAGGATGAGCTAATCCAGAAG |
| | | | | ATCCACAAACT |
| | | | | GGTGCAGAAGAGAGACTTCCTGGTGGACGATGCGGAGGTCGAGCGGTTAAG |
| 29798 | C2ORF27A | HSX2_01722550 | 61 | GCTTTTAGAGCACTGTGATGTAACATGTCAAGCAGAAATAGGGAGCATGTTTAC |
| | | | | AGCCATTCTAT |
| | | | | GAAAAAGTGTTCGGAATGTACAGACTAGCACAGAAGCTGGACTAATTGAACAAG |
| | | | | TATTGCTGAA AATGAGTGCTGTAGATGACATGATAGCAG |
| 84417 | C2ORF40 | 84417.001.1-T | 62 | AACTCAAGCTGATGCTTCAAAAACGAGAAGCACCTGTTCCAACTAAGACTAAAGT |
| | | | | GGCCGTTGAT |
| | | | | GAGAATAAAGCCAAAGAATTCCTTGGCAGCCTGAAGCGCCAGAAGCGGCAGCT |
| | | | | GTGGGACCGG |
| | | | | ACTCGGCCCGAGGTGCAGCAGTGGTACCAGCAGTTTCTCTACATGGGCTTTGAC |
| | | | | GAAGCGAAATT |
| | | | | TGAAGATGACATCACCTATTGGCTTAACAGAGATCGAAATGGACATGAATACTAT |
| | | | | GGCGATTACT |
| | | | | ACCAACGTCACTATGATGAAGACTCTGCAATTGGTCCCCGGAGCCCCTACGGCTT |
| | | | | TAGGCATGGA |
| | | | | GCCAGCGTCAACTACGATGACTACTAACCATGACTTGCCACACGCTGTACAAGAA |
| | | | | GCAAATAGCG |
| | | | | ATTCTCTTCATGTATCTCCTAATGCCTTACACTACTTGGTTTCTGATTTGCTCTATTT |
| | | | | CAGCAGATCT |
| | | | | TTCTACCTACTTTGGTGATCAAAAAAGAAGAGTTAAAACAACAC |
| 84417 | C2ORF40 | 84417.001.2-T | 63 | AACTCAAGCTGATGCTTCAAAAACGAGAAGCACCTGTTCCAACTAAGACTAAAGT |
| | | | | GGCCGTTGAT |
| | | | | GAGAATAAAGCCAAAGAATTCCTTGGCAGCCTGAAGCGCCAGAAGCGGCAGCT |
| | | | | GTGGGACCGG |
| | | | | ACTCGGCCCGAGGTGCAGCAGTGGTACCAGCAGTTTCTCTACATGGGCTTTGAC |
| | | | | GAAGCGAAATT |
| | | | | TGAAGATGACATCACCTATTGGCTTAACAGAGATCGAAATGGACATGAATACTAT |
| | | | | GGCGATTACT |
| | | | | ACCAACGTCACTATGATGAAGACTCTGCAATTGGTCCCCGGAGCCCCTACGGCTT |
| | | | | TAGGCATGGA |
| | | | | GCCAGCGTCAACTACGATGACTACTAACCATGACTTGCCACACGCTGTACAAGAA |
| | | | | GCAAATAGCG |
| | | | | ATTCTCTTCATGTATCTCCTAATGCCTTACACTACTTGGTTTCTGATTTGCTCTATTT |
| | | | | CAGCAGATCT |
| | | | | TTCTACCTACTTTGGTGATCAAAAAAGAAGAGTTAAAACAACAC |
| 84417 | C2ORF40 | HSX2_02098213 | 64 | AAATTTGAAGATGACATCACCTATTGGCTTAACAGAGATCGAAATGGACATGAAT |
| | | | | ACTATGGCGA |
| | | | | TTACTACCAACGTCACTATGATGAAGACTCTGCAATTGGTCCCCGGAGCCCCTAC |
| | | | | GGCTTTAGGC |
| | | | | ATGGAGCCAGCGTCAACTACGATGACTACTAACCATGACTTGCCACACGCTGTAC |
| | | | | AAGAAGCAAA |
| | | | | TAGCGATTCTCTTCATGTATCTCCTAATGCCTTACACTACTTGGTTTCTGATTTGCT |
| | | | | CTATTTCAGCA |
| | | | | GATCTTTTCTACCTACTTTGTGTGATCAAAAAAGAAGAGTTAAAACAACACATGT |
| | | | | AAATGCCTTTT |
| | | | | GATATTTCATGGGAATGCCTCTCATTTAAAAATAGAAATAAAGCATTTTGTTAAA |
| | | | | AAGAA |
| 720 | C4A | HSX2_00807218 | 65 | GCCAGGGACCTGGAAGATCTCAGCCCGATTCTCAGATGGCCTGGAATCCAACAG |
| | | | | CAGCACCCAG TTTGAGGTGAAGAAATATG |
| 721 | C4B | HSX2_00807494 | 66 | GCCAGGGACCTGGAAGATCTCAGCCCGATTCTCAGATGGCCTGGAATCCAACAG |
| | | | | CAGCACCCAG TTTGAGGTGAAGAAATATG |
| 721 | C4B | HSX2_00807696 | 67 | GCCAGGGACCTGGAAGATCTCAGCCCGATTCTCAGATGGCCTGGAATCCAACAG |
| | | | | CAGCACCCAG TTTGAGGTGAAGAAATATG |
| 100293534 | C4B_2 | HSX2_02455999 | 68 | GCCAGGGACCTGGAAGATCTCAGCCCGATTCTCAGATGGCCTGGAATCCAACAG |
| | | | | CAGCACCCAG TTTGAGGTGAAGAAATATG |
| 100293534 | C4B_2 | HSX2_02456123 | 69 | TGGATAAAAACCTGACTGTGAGCGTCCACGTGTC |
| 770 | CA11 | HSX2_00811047 | 70 | TGGATGGTGTCCCCCATGGTCGCTGAGACTCCCCTTCGAGGATTGCACCCGCCCG |
| | | | | TCCTAAGCCT |
| | | | | CCCCACAAGGCGAGGGGAGTTACCCCTAAAACAAAGCTATTAAAGGGACAGAAT |
| | | | | ACTT |
| 10241 | CALCOCO2 | HSX2_01482492 | 71 | CTTTTGAAGAGGGAGAACAGCAGATTGCTCAGTTACATGGGTCTGGATTTTAATT |
| | | | | CTTTGCCGTAT |
| | | | | CAAGTACCTACTTCAGATGAAGGAGGCGCAAGACAAAATCCAGGACTTGCCTAT |
| | | | | GGAAACCCAT ATTCTG |
| 55450 | CAMK2N1 | HSX2_01852736 | 72 | TTGTTATTGAAGATGATAGGATTGATGACGTGCTGAAAAATATGACCGACAAGG |
| | | | | CACCTCCTGGT |
| | | | | GTCTAACTCCCCCAAAGACAATGAGTTAAGGGAGAGAATAAGAACGGCGGTAA |
| | | | | CAGTTATTGGC |
| | | | | AAAAAGCATGAAAAGAGAAAGCACTTTGAAATTTATTACTAGCTTGCTACCCACG |
| | | | | ATGAAATCAA |
| | | | | CAACCTGTATCTGGTATCAGGCCGGGAGACAGATGAGGCGAGAGGAGGAGGA |
| | | | | GGAGGAGGAG |
| | | | | AAGGCTCTGGGCTCCTCTGCAAAAATAAAAATAAAAAAATAAATAAAATTTTAAA |
| | | | | AATAATAAAA |
| | | | | ATTCACTATATACACATATAAAGAAATAAAAAGAAGTCTCAGTTGCAGCTATTTG |
| | | | | TCAAAATTAAT |
| | | | | ATCCATTTCTTTTTATATACGGTGAATATTGCGCAATTATAGATCTGGATTTTGAA |
| | | | | CCACTTAATGA |
| | | | | AGCGGCAACACCAGGTGTTTTGAGGTGTTGGCATTCTTCGCTGATTTGGCTGTTC |
| | | | | CCAATGTTTAC |
| | | | | ATTATTTAATCTTGCAAAAATGGTTCTGTGCACTTGGATGTGAAATGCTGTCCAG |
| | | | | TTTTATTTTTTT |
| | | | | TATGTTGTTATCCTTGGATGTACAAAAAATTCAGAAAATGATCTCTGTAGATATTC |
| | | | | TGTTTTATTTT |
| | | | | GGTCATCTTTAGAAGTTATCAGGAATGTGTTTAAAACAAGAAGAGAACTTTTCTA |
| | | | | AGGAATGATA |
| | | | | CATAGAAAAGATTTTATTTTAAAATGAGTTGTAAAGCTTGTGTTTCTTTGTTGCTG |
| | | | | CAAGCTATCTG |
| | | | | CCCAAGTTAATGCAAATGGACACATTTTTTATGTCAGAAAAACACACACACACAC |
| | | | | ACACACACACA |
| | | | | CACACACACACGAAAAACAAAGAAAAAAATGCTTGAGCTTTTTCTAACTTCCCCT |
| | | | | TGCAGTCTGTT |
| | | | | GTGTGAGCAGCCTGTTTATTTCTCTAATATTATGTCAGTTTATTCTCTTTAATGGA |
| | | | | CTGTAAAAAAA |
| | | | | TGTAATCACAAGAGTGCCAAATATCTTGAAATGCCAAAAGGCATTTTAGTTTCTT |
| | | | | TTCTCTGTGCTC |
| | | | | TGAGTCCACGTACAGGAATGCTTGGAGTGTCTTTTCTGTTATTTATAGGGATTCT |
| | | | | CTTAAGGCACA |
| | | | | CCAGCTGCCTGTTTTGCATGGTATTTGCAAAAATGCCTCTTGCGTGAGGAAATCT |
| | | | | TTTACCATTTTT |
| | | | | TGTTTGCAACTTTGGACCTCAAGAGGTTTCCCTTCCCTTCCCCGTTCCCTCTTTTCT |
| | | | | TAATTCAATAT |
| | | | | TCTGTATGTTGCACCTTGAACCAGCACACAGGGCTATTTCTCCAATGTACAATAA |
| | | | | AAGAATTGTTC CTGTGTCTCA |
| 825 | CAPN3 | HSX2_00817525 | 73 | ATGGTAAAGATCAGCCTGTGGCTTTTACGTATTGTGCCAAAACATCCATCCTGAG |
| | | | | CCTGGAGAAG CTCTCACTCAACATTGCCACTGACTGGGAG |
| 825 | CAPN3 | HSX2_00817595 | 74 | GGAAGTTGAAAATACCATCTCCGTGGATCGGCCAGTG |
| 113201 | CASC4 | HSX2_02162192 | 75 | AGCAACTTGCTGAGCTTCGTCAGGAATTTCTTCGACAAGAAGACCAGCTTCAGG |
| | | | | ACTATAGGAAG AACAATACTTACCTTGTGAAGAGGTTAGAATATGA |
| 836 | CASP3 | HSX2_00819290 | 76 | GTGGGTGTGCCCTGCACCTGCCTCTTCCCCCATTCTCA |
| 836 | CASP3 | HSX2_02027519 | 77 | GACCCAGGACTTAATTACCCAGTGCCCAGTTGTG |
| 57639 | CCDC146 | HSX2_01937063 | 78 | AAAATTATATCAGAAATGGAGTCTAAGTTAGTAGAACAACAACTTGCAGAAGAA |
| | | | | AACAAGCTTTT |
| | | | | AAAGGAGCAAGAAAACATGAAAGAGCTAGTAGTCAACCTTCTCCGCATGACTCA |
| | | | | AATCAAAATTG |
| | | | | ATGAAAAGGAACAAAAGTCCAAGGATTTCCTGAAAGCTCAG |
| 57639 | CCDC146 | HSX2_01937168 | 79 | ATGAATGGCTATCAAAGAAGGATCAAAAATGCAACTGAGAAAATGATGGCTCTT |
| | | | | GTTGCTGAGCT |
| | | | | GTCCATGAAACAAGCCCTAACCATTGAACTCCAAAAGGAAGTCAGGGAGAAAGA |
| | | | | AGACTTCATCT |
| | | | | TCACTTGCAATTCCAGGATAGAAAAAGGTCTGCCACTCAATAAGGAAATTGAGA |
| | | | | AAGAATGGTTG |
| | | | | AAAGTCCTTCGAGATGAAGAAATGCACGCCTTGGCCATCGCTGAAAAGTCTCAG |
| 154467 | CCDC167 | HSX2_02255236 | 80 | ATCGATGGGCTAGAGGAGAAGCTGTCCCAGTGTCGGAGAGACCTGGAGGCCGT |
| | | | | GAACTCCAGA CTCCACAGCCGGGAGCTGAGCCCAGAGGCCAG |
| 146439 | CCDC64B | HSX2_02233673 | 81 | CTCACTTTCCTCTTCAGGCCAATGGAGCGACAGGGCCCAGATTGTCTTCCAAAAG |
| | | | | AAGCTGATGC |
| | | | | CCTCCCCGCCCCAGGATGCTGGGCAAGGGCTCATCGGGAGCAGGGGCTCATCCG |
| | | | | GAGCAGGGG |
| | | | | CCAGCTTTGTGGGGCAGGAGCTCCAGGTGGGTGGCAGGGGCAGGTGAACAGA |
| | | | | GCTATTTTCCGAATTAATATA |
| 114800 | CCDC85A | HSX2_02167533 | 82 | TTTTTTTTTCATCTTAATGAGTTTCTCATAAAGACACATCTTGGGGCATACCAACC |
| | | | | TTCATATTCATT |
| | | | | CAGTGTATCCATTGGTGTTAATATTAACAATTTCAACTAAAAACAACAAACATAA |
| | | | | TTTTTCTAAGTG |
| | | | | TTTCGAGCTGTGAGATGTCAATTTCTACAATTTAGTTTCTAGACATGCTGTATATT |
| | | | | TAATAAACTTT |
| | | | | ATGTAAACTTTAAAATATTGCACTGCATTTATGAAAAAAGCTTTCTTTGCCTACTG |
| | | | | CAGCTATCTAA |
| | | | | CATTTTATGAAACTGACGCATGTCCTTCATTATTGAGGCTAAAAGCTCTTGTTCAG |
| | | | | ATTGCTTGAG |
| | | | | GTTTGAAAAAGAGGTGTGCTAGCTTTGCTTAGTTTATTTCTTATTTTTGGTATACA |
| | | | | TATACATATAT |
| | | | | ATAACATTAGAATGTGTGTACTGGAAATGCTATGATAGGTATTTTGTGTTAATCC |
| | | | | AAATGCAATGA |
| | | | | TAGTTTCTTGTATGAATGTGCAAGAGGCCTGTGACCGAATGCTACGGTTGTGTG |
| | | | | GAGGAAACTTG |
| | | | | GAGATGCTGCAGGTTCGTGTCCTGGAATTAGGCAACATTTGTCAGGAAACCAGT |
| | | | | ACAAAGGACC |
| | | | | AATGTGAGATGCACTCTTTTTCAAACAGGAGATCACCACTGCCAGAAAGTGATA |
| | | | | GAAGACAAGAA |
| | | | | GAAAAAGGAAAGAGTGGGTTTCCACAAACCTGGACTCATGGAATAACATGGAG |
| | | | | TGATTGTACAT |
| | | | | TGCACATATCTCCCCTCTAAAACCTGTAGTACAACTTCTCCCCTCAAGCTGATATT |
| | | | | CTGTGTCTCTC |
| | | | | ACCTCAATGTCCACAACAGTCAATCTCAAACAAGGTAGCTTTGAACATCTCAAAC |
| | | | | AGAGTAGCTTT |
| | | | | GAAAATCAACATTTTGGTACCAGTGTTTTCATTAGAAATAAGTGTTTTTAACTCCC |
| | | | | CAGATATAATA |
| | | | | TTGTAATTGGGAAACCTGAATAGTTTTAAGAGTCCAGTGGAGCCTCAGTGTTTTG |
| | | | | AATAGAGATG |
| | | | | ACTCATTACACATAATGGTAGGAGTTTTCTACCATATCATATTGAAGATATGAAG |
| | | | | AGGTATTTTCC |
| | | | | AGCTTCCCAAGTTTGAGAGTTGGGCATTTACCTCTTACACTTCAGTCAACAAACA |
| | | | | AACTAGTAATT |
| | | | | CCTAATGCATAATTAATTTGGATGCAAGATAAGATTAAGTTTCTGGAGTTTTGTTT |
| | | | | TGACTTCTGA |
| | | | | GGTGCTAAAAAATACCATGTAAATTCTCCCTACCAGCTAGAAGGGTAGCAAGAC |
| | | | | GTTGTAGAGTG |
| | | | | CTTATCAGAGAGTACCATTTACTTCAATTGATCAATATAAATATATC |
| 83605 | CCM2 | HSX2_02073060 | 83 | CCACATCCAGTTCCACCACCAATGGGAACAGGGCCACGGGCAGCTCTGATGACC |
| | | | | GGTCGGCACC |
| | | | | CTCAGAGGGGGATGAGTGGGACCGCATGATCTCGGACATCAGCAGCGACATTG |
| | | | | AGGCGCTGGG |
| | | | | CTGCAGCATGGACCAGGACTCAGCATGATGGACAGTGGATGGGGGGGCACCCA |
| | | | | CACCTTCCGCG CAGTCGTCATAGGCCTTCCCAGAAGGAGCTGCC |
| 894 | CCND2 | HSX2_00822288 | 84 | GAGTGGGAACTGGTGGTGCTGGGGAAGTTGAAGTGGAACCTGGCAGCTGTCAC |
| | | | | TCCTCATGACT |
| | | | | TCATTGAGCACATCTTGCGCAAGCTGCCCCAGCAGCGGGAGAAGCTGTCTCTGAT |
| | | | | CCGCAAGCAT GCTCAGACCTTCATTGCTCTGTGTGCCACCG |
| 645121 | CCNI2 | HSX2_02420442 | 85 | GTTGGTGATATGCAGTACAGCTGCTGCAAGGAACTTGTAATGCAGCAACTGAGA |
| | | | | AGTCTTCAGTC |
| | | | | ATCCTCCTGCACAGACAACTTTGTGTCACCTGCCAACTAGCCCCTCTGCCTCCACC |
| | | | | CCGGGGCTTT |
| | | | | CAGAGCATAGTGTGAAACCTCCTTGCTTGGACTACCATGAGTTCTTTGGCTTGTTA |
| | | | | TGAATCCTGT |
| | | | | AAAAAGGGAAGGTGGCTCTGGAAGAGCAACTGAGAAAAAGTTCCCAACTGAGC |
| | | | | CCTTGGAAAAA |
| | | | | AAATAAAGGGGAGAGGGGAAAGGCAGGCTGAGGTCAGTAGAGGTCAGGGTGG |
| | | | | TCTGATAGACT |
| | | | | ATGACCCTGTCTTCCCTTTTTCTCCTCTGGGCCTGAAGCCAGGGAGTATGAATGA |
| | | | | ATGTTCAAATG |
| | | | | GCAGCTTGTTTTACCTTCCTTCTCCAGCAAGGGTTGGCATTGGCCCCTGGGAGCG |
| | | | | CTGGAATATG |
| | | | | AAACCAAGAGGCAGGCCTGGCTCAGGGCTGAAGGGCTGGGGCTAGCTCTGACC |
| | | | | AGTTCTTGCTG |
| | | | | CTCTACCTGCCAGAGCTGGCCAGGTCCTTCCAACTTCCCAGGCTTATCCCAAACAC |
| | | | | ATATGTGGTG CTTCCAGTCCCAACTACCCTGCC |
| 283316 | CD163L1 | HSX2_02326932 | 86 | CTTCCAGGGCTCACCAGATCAACCTCTAAATATC |
| 961 | CD47 | HSX2_00827007 | 87 | AAAGCTGTAGAGGAACCCCTTAATG |
| 961 | CD47 | HSX2_00827010 | 88 | CTATACAACCTCCTAGGAAAGCTGTAGAGGAACC |
| 1014 | CDH16 | HSX2_00832581 | 89 | ACCATGGCTTGATCGTGAGTGGACCCAGCAAGGACCCCGATCTGGCCAGTGGGC |
| | | | | ACGGTCCCTA |
| | | | | CAGCTTCACCCTTGGTCCCAACCCCACGGTGCAACGGGATTGGCGCCTCCAGACT |
| | | | | CTCAATG |
| 1001 | CDH3 | HSX2_00831293 | 90 | GGTTTGGATTTTGAGGCCAAAAACCAGCACACCCTGTACGTTGAAGTGACCAAC |
| | | | | GAGGCCCCTTT |
| | | | | TGTGCTGAAGCTCCCAACCTCCACAGCCACCATAGTGGTCCACGTGGAGGATGT |
| | | | | GAATGAGGCA |
| | | | | CCTGTGTTTGTCCCACCCTCCAAAGTCGTTGAGGTCCAGGAGGGCATCCCCACTG |
| | | | | GGGAGCCTGT |
| | | | | GTGTGTCTACACTGCAGAAGACCCTGACAAGGAGAATCAAAAGATCAG |
| 1001 | CDH3 | HSX2_00831298 | 91 | CTACCGCATCCTGAGAGACCCAGCAGGGTGGCTAGCCATGGACCCAGACAGTGG |
| | | | | GCAGGTCACA |
| | | | | GCTGTGGGCACCCTCGACCGTGAGGATGAGCAGTTTGTGAGGAACAACATCTAT |
| | | | | GAAGTCATGG TCTTGGCCATGGACAATG |
| 1001 | CDH3 | HSX2_00831310 | 92 | GTGACACAGTGGTCTTGTCCCTGAAGAAGTTCCTGAAGCAGGATACATATGACG |
| | | | | TGCACCTTTCT |
| | | | | CTGTCTGACCATGGCAACAAAGAGCAGCTGACGGTGATCAGGGCCACTGTGTGC |
| | | | | GACTGCCATG |
| | | | | GCCATGTCGAAACCTGCCCTGGACCCTGGAAGGGAGGTTTCATCCTCCCTGTGCT |
| | | | | GGGGGCTGTC CTGGCTCTGCTGT |
| 1001 | CDH3 | HSX2_00831320 | 93 | GACTATGACATCACCCAGCTCCACCGAGGTCTGGAGGCCAGGCCGGAGGTGGTT |
| | | | | CTCCGCAATG |
| | | | | ACGTGGCACCAACCATCATCCCGACACCCATGTACCGTCCTCGGCCAGCCAACCC |
| | | | | AGATGAAATC GGCAACTTTA |
| 1001 | CDH3 | HSX2_00831328 | 94 | CTGCTGTGACTGACCTACAGTGGACTTTCTCTCTGGAATGGAACCTTCTTAGGCCT |
| | | | | CCTGGTGCAA |
| | | | | CTTAATTTTTTTTTTTAATGCTATCTTCAAAACGTTAGAGAAAGTTCTTCAAAAGTG |
| | | | | CAGCCCAGAG |
| | | | | CTGCTGGGCCCACTGGCCGTCCTGCATTTCTGGTTTCCAGACCCCAATGCCTCCCA |
| | | | | TTCGGATGGA |
| | | | | TCTCTGCGTTTTTATACTGAGTGTGCCTAGGTTGCCCCTTATTTTTTATTTTCCCTG |
| | | | | TTGCGTTGCTA |
| | | | | TAGATGAAGGGTGAGGACAATCGTGTATATGTACTAGAACTTTTTTATTAAAGAA |
| | | | | ACTTTTCCCA |
| 1001 | CDH3 | HSX2_00831335 | 95 | GCAACTTTATAATTGAGAACCTGAAGGCGGCTAA |
| 1001 | CDH3 | HSX2_02495403 | 96 | GACTATGACATCACCCAGCTCCACCGAGGTCTGGAGGCCAGGCCGGAGGTGGTT |
| | | | | CTCCGCAATG |
| | | | | ACGTGGCACCAACCATCATCCCGACACCCATGTACCGTCCTCGGCCAGCCAACCC |
| | | | | AGATGAAATC GGCAACTTTATAATTGAG |
| 1001 | CDH3 | HSX2_02495404 | 97 | AACCTGAAGGCGGCTAACACAGACCCCACAGCCCCGCCCTACGACACCCTCTTGG |
| | | | | TGTTCGACTA |
| | | | | TGAGGGCAGCGGCTCCGACGCCGCGTCCCTGAGCTCCCTCACCTCCTCCGCCTCC |
| | | | | GACCAAGACC |
| | | | | AAGATTACGATTATCTGAACGAGTGGGGCAGCCGCTTCAAGAAGCTGGCAGACA |
| | | | | TGTACGGTGG |
| | | | | CGGGGAGGACGACTAGGCGGCCTGCCTGCAGGGCTGGGGACCAAACGTCAGGC |
| | | | | CACAGAGCAT |
| | | | | CTCCAAGGGGTCTCAGTTCCCCCTTCAGCTGAGGACTTCGGAGCTTGTCAGGAAG |
| | | | | TGGCCGTAGC |
| | | | | AACTTGGCGGAGACAGGCTATGAGTCTGACGTTAGAGTGGTGGCTTCCTTAGCC |
| | | | | TTTCAGGATGG |
| | | | | AGGAATGTGGGCAGTTTGACTTCAGCACTGAAAACCTCTCCACCTGGGCCAGGG |
| | | | | TTGCCTCAGA |
| | | | | GGCCAAGTTTCCAGAAGCCTCTTACCTGCCGTAAAATGCTCAACCCTGTGTCCTG |
| | | | | GGCCTGGGCC |
| | | | | TGCTGTGACTGACCTACAGTGGACTTTCTCTCTGGAATGGAACCTTCTTAGGCCTC |
| | | | | CTGGTGCAAC |
| | | | | TTAATTTTTTTTTTTAATGCTATCTTCAAAACGTTAGAGAAAGTTCTTCAAAAGTGC |
| | | | | AGCCCAGAGC |
| | | | | TGCTGGGCCCACTGGCCGTCCTGCATTTCTGGTTTCCAGACCCCAATGCCTCCCAT |
| | | | | TCGGATGGAT |
| | | | | CTCTGCGTTTTTATACTGAGTGTGCCTAGGTTGCCCCTTATTTTTTATTTTCCCTGT |
| | | | | TGCGTTGCTAT |
| | | | | AGATGAAGGGTGAGGACAATCGTGTATATGTACTAGAACTTTTTTATTAAAGAAA |
| | | | | CTTTTCCCA |
| 55755 | CDK5RAP2 | HSX2_01874332 | 98 | CCTGAGGTGCCCCTTCAGCCTGACAAACACG |
| 55755 | CDK5RAP2 | HSX2_01874383 | 99 | TTTCAGAGACTCCTCCACTCTCTGGGAATGACACGGACTCCCTCTCCTGCGACAGT |
| | | | | GGCAGTTCG |
| | | | | GCAACTAGCACTCCGTGTGTGTCCCGCCTGGTCACTGGCCACCACCTGTGGGCCA |
| | | | | GCAAGAATG |
| | | | | GCCGCCATGTCCTGGGCCTGATTGAGGACTATGAGGCCCTGCTCAAACAGATCA |
| | | | | GCCAGGGACA |
| | | | | GAGGCTCCTTGCTGAAATGGACATTCAAACCCAAGAGGCTCCCAGCTCCACAAGT |
| | | | | CAAGAGCTG GGAACAAAG |
| 55755 | CDK5RAP2 | HSX2_01874417 | 100 | ATTGGAGAAATGAAGGCAGAGGTCACCAAACTACATAAAAAATTGTTTGAACAA |
| | | | | GAAAAGAAGT |
| | | | | TGCAAAACACCATGAAGCTTTTGCAGCTGAGCAAGCGCCAGGAAAAAGTCATCT |
| | | | | TTGATC |
| 57214 | CEMIP | HSX2_01916997 | 101 | GCTGTTGTTCCTGAAGCTGAAAGCTCAGAACGAGAGAGAGAAGTTTGCTTTCTG |
| | | | | CTCCATGAAAG |
| | | | | GCTGTGAGAGGATAAAGATTAAAGCTCTGATTCCAAAGAACGCAGGCGTCAGTG |
| | | | | ACTGCACAGC |
| | | | | CACAGCTTACCCCAAGTTCACCGAGAGGGCTGTCGTAGACGTGCCGATGCCCAA |
| | | | | GAAGCTCTTTG GTTCTCAGCTG |
| 8869 | ST3GAL5 | HSX2_01367269 | 102 | AGAGCTCAGAAATATGCTCAGCAAGTCTTGCAGAAGGAATGTCGTCCCAAGTTT |
| | | | | GCCAAGACATC |
| | | | | AATGGCGCTGTTATTTGAGCACAGGTATAGCGTGGACTTACTCCCTTTTGTGCAG |
| 8869 | ST3GAL5 | HSX2_01367270 | 103 | AAGGCCCCCAAAGACAGTGAAGCTGAGTCCAAGTACGATCCTCCTTTTGGGTTC |
| | | | | CGGAAGTTCTC |
| | | | | CAGTAAAGTCCAGACCCTCTTGGAACTCTTGCCAGAGCACGACCTCCCTGAACAC |
| | | | | TTGAAAGCCA |
| | | | | AGACCTGTCGGCGCTGTGTGGTTATTGGAAGCGGAGGAATACTGCACGGATTA |
| | | | | GAACTGGGCCA CACCCTGAACCAGTTCGATGTTGTGATAAG |
| 8869 | ST3GAL5 | HSX2_01367276 | 104 | GTTAAACAGTGCACCAGTTGAGGGATATTCAGAACATGTTGGAAATAAAACTAC |
| | | | | TATAAGGATGA |
| | | | | CTTATCCAGAGGGCGCACCACTGTCTGACCTTGAATATTATTCCAATGACTTATTT |
| | | | | GTTGCTGTTTT |
| | | | | ATTTAAGAGTGTTGATTTCAACTGGCTTCAAGCAATGGTAAAAAAGGAAACCCT |
| | | | | G |
| 8869 | ST3GAL5 | HSX2_01367281 | 105 | CCATTCTGGGTACGACTCTTCTTTTGGAAGCAGGTGGCAGAAAAAATCCCACTGC |
| | | | | AGCCAAAACA |
| | | | | TTTCAGGATTTTGAATCCAGTTATCATCAAAGAGACTGCCTTTGACATCCTTCAGT |
| | | | | ACTCAGAGCC TCAGTCAAGGTTCTGGGGCCGAGATAAG |
| 4435 | CITED1 | HSX2_01064533 | 106 | CTATAAAAATGTGCCCGGCGGCAAAGCTGCAGAAGGGGCACCGCTGAGCTGCC |
| | | | | GAGAGGAAGC |
| | | | | TCGCTCTGCCCGGCTGCCCTCTTGTAGTCCGCCGGCGAGGGGCAGTTCTCGGTG |
| | | | | AGGAGGAAGA GAGC |
| 4435 | CITED1 | HSX2_01064535 | 107 | GCACAGCACCCGCGCGGGCCCTCC |
| 4435 | CITED1 | HSX2_01064539 | 108 | CACAACAGCTCCAGCTGGCAGCATCACTTCCCGCCAATTTATCCAACTTCTGCCA |
| | | | | AGGCTCTGAAA |
| | | | | TGCCAACAACGTCGAGGCCTGCACTTGATGTCAAGGGTGGCACCTCACCTGCGA |
| | | | | AGGAG |
| 4435 | CITED1 | HSX2_01064543 | 109 | GATGCCAACCAAGAGATGAGCTCCGTGGCCTACTCCAACCTTGCGGTGAAAGAT |
| | | | | CGCAAAGCAG TGGCCATTCTGCA |
| 4435 | CITED1 | HSX2_01064546 | 110 | GCCAGTGGGGCTCCCACTAGTTCCTCGGGATCTCCAATAGGCTCTCCTACAACCA |
| | | | | CCCCTCCCACT AAA |
| 4435 | CITED1 | HSX2_01064547 | 111 | CCCCCATCCTTCAACCTGCACCCCGCCCCTCACTTGCTGGCTAGTATGCACCTGCA |
| | | | | GAAACTTAAT |
| | | | | AGCCAGTATCAGGGGATGGCTGCTGCCACTCCAGGCCAACCCGGGGAGGCAGG |
| | | | | ACCCCTGCAA |
| | | | | AACTGGGACTTTGGGGCCCAGGCGGGAGGGGCAGAATCACTCTCTCCTTCTGCT |
| | | | | GGTGCCCAG |
| 4435 | CITED1 | HSX2_01064548 | 112 | AGCCCTGCTATCATCGATTCGGACCCAGTGGATGAGGAAGTGCTGATGTCGCTG |
| | | | | GTGGTGGAAC |
| | | | | TGGGGTTGGACCGAGCCAATGAGCTTCCGGAGCTGTGGCTGGGGCAGAATGAG |
| | | | | TTTGACTTCAC |
| | | | | TGCGGACTTTCCATCTAGCTGCTAATGCCAAGTGTCCCTAAAGATGGAGGAATA |
| | | | | AAGCCACCAAT TCTGTTGTAAATAAAAATAAAGTTACTTACAA |
| 4435 | CITED1 | HSX2_01064567 | 113 | GAGTCCCTGCTTCGCAGAGCACCGGACTTGGAGTCAGAGAGACCTGGCTGCGA |
| | | | | ATCCCGGCTCC GCCACTTTCTAGCGCTGTGACCTTGG |
| 4435 | CITED1 | HSX2_01064569 | 114 | TTTTCAGGTGGCGAAACTGACTGGTCTGGTTAGAGCGCGTCCAAGCCAAGCCGC |
| | | | | AGTGCTGTTTG |
| | | | | GATTTTTAAATTTCTCCTTCTGAGTGAGAAAATAGCAATCGAGATGGAACCATCC |
| | | | | G |
| 4435 | CITED1 | HSX2_01064572 | 115 | TAGCGCTGTGACCTTGGTTTTCAGGTGGCGAAAC |
| 4435 | CITED1 | HSX2_01064573 | 116 | CGAGATGGAACCATCCGCACAACAGCTCCAGCTG |
| 4435 | CITED1 | HSX2_01064574 | 117 | TAGCGCTGTGACCTTGGCACAACAGCTCCAGCTG |
| 4435 | CITED1 | HSX2_02509162 | 118 | CTATAAAAATGTGCCCGGCGGCAAAGCTGCAGAAGGGGCACCGCTGAGCTGCC |
| | | | | GAGAGGAAGC |
| | | | | TCGCTCTGCCCGGCTGCCCTCTTGTAGTCCGCCGGCGAGGGGCAGTTCTCGGTG |
| | | | | AGGAGGAAGA GAGCAGCGGACGGCACAGCACCCGCGCGGGCCCTCC |
| 4435 | CITED1 | HSX2_02509166 | 119 | GATGCCAACCAAGAGATGAGCTCCGTGGCCTACTCCAACCTTGCGGTGAAAGAT |
| | | | | CGCAAAGCAG |
| | | | | TGGCCATTCTGCACTACCCTGGGGTAGCCTCAAATGGAACCAAGGCCAGTGGGG |
| | | | | CTCCCACTAGT |
| | | | | TCCTCGGGATCTCCAATAGGCTCTCCTACAACCACCCCTCCCACTAAACCCCCATC |
| | | | | CTTCAACCTGC |
| | | | | ACCCCGCCCCTCACTTGCTGGCTAGTATGCACCTGCAGAAACTTAATAGCCAGTA |
| | | | | TCAGGGGATG |
| | | | | GCTGCTGCCACTCCAGGCCAACCCGGGGAGGCAGGACCCCTGCAAAACTGGGA |
| | | | | CTTTGGGGCCC |
| | | | | AGGCGGGAGGGGCAGAATCACTCTCTCCTTCTGCTGGTGCCCAGAGCCCTGCTA |
| | | | | TCATCGATTCG |
| | | | | GACCCAGTGGATGAGGAAGTGCTGATGTCGCTGGTGGTGGAACTGGGGTTGGA |
| | | | | CCGAGCCAAT |
| | | | | GAGCTTCCGGAGCTGTGGCTGGGGCAGAATGAGTTTGACTTCACTGCGGACTTT |
| | | | | CCATCTAGCTG |
| | | | | CTAATGCCAAGTGTCCCTAAAGATGGAGGAATAAAGCCACCAATTCTGTTGTAA |
| | | | | ATAAAAATAAA GTTACTTACAA |
| 1188 | CLCNKB | HSX2_00842841 | 120 | ATCAGTGTCGCCCCCAGGCGTCCTGTTCAGCATC |
| 9076 | CLDN1 | HSX2_01381993 | 121 | GCACATTGCAAGCAACCCGTGCCTTGATGGTGGTTGGCATCCTCCTGGGAGTGA |
| | | | | TAGCAATCTTT |
| | | | | GTGGCCACCGTTGGCATGAAGTGTATGAAGTGCTTGGAAGACGATGAGGTGCA |
| | | | | GAAGATGAGG ATGGCTGTCATTGGGGGTGCGATATTTCTTCTTGCAG |
| 9076 | CLDN1 | HSX2_01381997 | 122 | GTCTGGCTATTTTAGTTGCCACAGCATGGTATGGCAATAGAATCGTTCAAGAATT |
| | | | | CTATGACCCTA TGACCCCAGTCAATGCCAG |
| 9076 | CLDN1 | HSX2_01382002 | 123 | GTACGAATTTGGTCAGGCTCTCTTCACTGGCTGGGCTGCTGCTTCTCTCTGCCTTC |
| | | | | TGGGAGGTGC |
| | | | | CCTACTTTGCTGTTCCTGTCCCCGAAAAACAACCTCTTACCCAACACCAAGGCCCT |
| | | | | ATCCAAAACC |
| | | | | TGCACCTTCCAGCGGGAAAGACTACGTGTGACACAGAGGCAAAAGGAGAAAAT |
| | | | | CATGTTGAAAC |
| | | | | AAACCGAAAATGGACATTGAGATACTATCATTAACATTAGGACCTTAGAATTTTG |
| | | | | GGTATTGTAAT |
| | | | | CTGAAGTATGGTATTACAAAACAAACAAACAAACAAAAAACCCATGTGTTAAAAT |
| | | | | ACTCAGTGCT |
| | | | | AAACATGGCTTAATCTTATTTTATCTTCTTTCCTCAATATAGGAGGGAAGATTTTTC |
| | | | | CATTTGTATTA |
| | | | | CTGCTTCCCATTGAGTAATCATACTCAACTGGGGGAAGGGGTGCTCCTTAAATAT |
| | | | | ATATAGATATG |
| | | | | TATATATACATGTTTTTCTATTAAAAATAGACAGTAAAATACTATTCTCATTATGTT |
| | | | | GATACTAGCA |
| | | | | TACTTAAAATATCTCTAAAATAGGTAAATGTATTTAATTCCATATTGATGAAGATG |
| | | | | TT |
| 149461 | CLDN19 | HSX2_02242186 | 124 | ACACCTGTCAATGCCAGGTATGAATTTGGCCCAG |
| 53405 | CLIC5 | HSX2_01785484 | 125 | AACACCGGGAATCCAACACAGCGGGCATCGACAT |
| 79745 | CLIP4 | HSX2_02019683 | 126 | CTTTCTTTGATCCTAATGATGCATCATGCCAGGAAATTCTTTTTGATCCCAAAACTT |
| | | | | CAGTTTCAGA |
| | | | | ATTATTTGCCATTTTGAGACAGTGGGTTCCTCAGGTCCAACAAAACATTGACATTA |
| | | | | TTGGAAATGA G |
| 79745 | CLIP4 | HSX2_02019711 | 127 | ATTGACAGATATGACTCTTTTACATTATACCTGCAAATCTGGAGCTCATGGTAT |
| 79745 | CLIP4 | HSX2_02019770 | 128 | ATGTGGATGCCACTTGCAGTGATTTTAATTTTGGAACAGCTTTGCATATTGCAGC |
| | | | | ATACAACTTGT |
| | | | | GTGCAGGTGCTGTGAAGTGCCTCTTGGAGCAGGGAGCAAATCCTGCATTTAGG |
| 1271 | CNTFR | HSX2_02496357 | 129 | GCATGGCTCCAAAATTATGGTCTGTGAGAAGGACCCAGCCCTCAAGAACCGCTG |
| | | | | CCACATTCGCT |
| | | | | ACATGCACCTGTTCTCCACCATCAAGTACAAGGTCTCCATAAGTGTCAGCAATGC |
| | | | | CCTGGGCCAC AATGCCACAGCTATCACCTTTGACGAGTTCACCATTG |
| 8506 | CNTNAP1 | HSX2_01339423 | 130 | AAGAGCGGAGGACCAGGAACCAGAGAGAGAGAGAGAGAAAAGAGAGAGGAG |
| | | | | AGACAGAGCG |
| | | | | CTTGGGGGCGAAAGGAGAGAGGGAGGGAAGGGTGGGTAAGGAGGAGAGAGC |
| | | | | GGTCTGCTGC |
| | | | | AAACCCCAGGAGGAGAGCTTGGAGCCCAAGCCAGAACTCGAGCCCTAGCCGGA |
| | | | | GCCGTTCACA |
| | | | | GGGAGGCGGCTGCCGGGACCGTCAGCCCTGCATGATGCATCTCCGGCTCTTCTG |
| | | | | CATCCTGCTCG CCGCGGTCTCAGGAGCCGAGGGCTGGGGCTACT |
| 1281 | COL3A1 | HSX2_00847965 | 131 | GGTAACCCAGGACCCCCAGGTCCCAGCGGTTCTCCAGGCAAGGATGGGCCCCCA |
| | | | | GGTCCTGCGG |
| | | | | GTAACACTGGTGCTCCTGGCAGCCCTGGAGTGTCTGGACCAAAAGGTGATGCTG |
| | | | | GCCAACCAGG AGAGAAGGGATCGCCTGGTGCCCAGGGCCCACCA |
| 1290 | COL5A2 | HSX2_00850255 | 132 | GCCCAAAAGGGGAACCAGGGCCACATGGTATTCA |
| 50509 | COL5A3 | HSX2_01735527 | 133 | GGGAATACTGGATTGACCCCAACCAGGGCTGCGCGCGGGACTCGTTCAGGGTTT |
| | | | | TTTGCAACTTC |
| | | | | ACGGCGGGAGGAGAGACCTGCCTCTATCCCGACAAGAAGTTTGAGATC |
| 6271 | S100A1 | HSX2_01205429 | 134 | GCCACATTTGCAACCTTGGCCATCTGTCCAGAACCTGCTCCCACCTCAGGCCCAG |
| | | | | GCCAACC |
| 6271 | S100A1 | HSX2_01205433 | 135 | GTGCACTGCTGCAATGGGCTCTGAGCTGGAGACGGCGATGGAGACCCTCATCAA |
| | | | | CGTGTTCCAC |
| | | | | GCCCACTCGGGCAAAGAGGGGGACAAGTACAAGCTGAGCAAGAAGGAGCTGAA |
| | | | | AGAGCTGCTGCAGACGGAGCTCTCTGGCTTCCTGGAT |
| 6271 | S100A1 | HSX2_01205437 | 136 | GCCCAGAAGGATGTGGATGCTGTGGACAAGGTGATGAAGGAGCTAGACGAGAA |
| | | | | TGGAGACGG |
| | | | | GGAGGTGGACTTCCAGGAGTATGTGGTGCTTGTGGCTGCTCTCACAGTGGCCTG |
| | | | | TAACAATTTCT |
| | | | | TCTGGGAGAACAGTTGAGCAGACAGCCACATTGGGCAGCGCCCTTCCTCTCCACC |
| | | | | CTCCCAGACC |
| | | | | TGCCTCTTCCCCCTGCTTCCACCTCACCCCACTTATCCCTCTCCATAACCCCACCCTT |
| | | | | GCCCACCCC |
| | | | | ACCCCCACCCCCACCAAGGGCGCAAGAGTAGCGGTCCAAGCCTGCAACTCATCTT |
| | | | | TCATTAAAGG CTTCTCTCTCACCAGC |
| 64478 | CSMD1 | HSX2_01977629 | 137 | CGGAATGTGGTGGTCAGATCCATGCAGCCACATCAGGACGAATATTGTCCCCTG |
| | | | | GCTATCCAGCT |
| | | | | CCGTATGACAACAACCTCCACTGCACCTGGATTATAGAGGCAGACCCAGGAAAG |
| | | | | ACCATTAG |
| 1465 | CSRP1 | HSX2_00864524 | 138 | TGCAAGAAGAATCTGGACAGTACCACTGTGGCCGTGCATGGTGAGGAG |
| 1474 | CST6 | HSX2_00864973 | 139 | GAGGGCTCCGACGGCACTGACGGCCATGGCGCGTTCGAACCTCCCGCTGGCGCT |
| | | | | GGGCCTGGCC |
| | | | | CTGGTCGCATTCTGCCTCCTGGCGCTGCCACGCGACGCCCGGGCCCGGCCGCAG |
| | | | | GAGCGCATGG |
| | | | | TCGGAGAACTCCGGGACCTGTCGCCCGACGACCCGCAGGTGCAGAAGGCGGCG |
| | | | | CAGGCGGCCG |
| | | | | TGGCCAGCTACAACATGGGCAGCAACAGCATCTACTACTTCCGAGACACGCACAT |
| | | | | CATCAAGGCG CAGAGCCAG |
| 1499 | CTNNB1 | HSX2_00866738 | 140 | CTGATTTGATGGAGTTGGACATGGCCATGGAACCAGACAGAAAAGCGGCTGTTA |
| | | | | GTCACTGGCA |
| | | | | GCAACAGTCTTACCTGGACTCTGGAATCCATTCTGGTGCCACTACCACAGCTCCTT |
| | | | | CTCTGAGTGG |
| | | | | TAAAGGCAATCCTGAGGAAGAGGATGTGGATACCTCCCAAGTCCTGTATGAGTG |
| | | | | GGAACAGGGA TTTTCTCAGTCCTTCA |
| 1499 | CTNNB1 | HSX2_00866791 | 141 | CTCTTCGTCATCTGACCAGCCGACACCAAGAAGCAGAGATGGCCCAGAATGCAG |
| | | | | TTCGCCTTCAC |
| | | | | TATGGACTACCAGTTGTGGTTAAGCTCTTACACCCACCATCCCACTGGCCTCTGAT |
| | | | | AAAG |
| 1499 | CTNNB1 | HSX2_00866821 | 142 | ACTGCTGATCTTGGACTTGATATTGGTGCCCAGGGAGAACCCCTTGGATATCGCC |
| | | | | AGGATG |
| 1499 | CTNNB1 | HSX2_02497461 | 143 | CTCATCATACTGGCTAGTGGTGGACCCCAAGCTTTAGTAAATATAATGAGGACCT |
| | | | | ATACTTACGAA |
| | | | | AAACTACTGTGGACCACAAGCAGAGTGCTGAAGGTGCTATCTGTCTGCTCTAGTA |
| | | | | ATAAGCCGGC TATTGTAGAAGCTG |
| 1508 | CTSB | HSX2_00867649 | 144 | AGAATCACGTCCTGGAACTGCATGTTCTTGCGACTCTTGGGACTTCATCTTAACTT |
| | | | | CTCGCTGCCC |
| | | | | CAGCCATGTTTTCAACCATGGCATCCCTCCCCCAATTAGTTCCCTGT |
| 7050 | TGIF1 | HSX2_02521755 | 145 | GTCTGTAACTGGTTCATCAACGCCCGCCGCAGGCTCCTCCCTGACATGCTGAGAA |
| | | | | AGGATGGCAA |
| | | | | AGATCCAAATCAGTTCACAATTTCCCGCCGTGGGGCCAAGATTTCTGAAACGAGC |
| | | | | TCTGTGGAGT |
| | | | | CCGTGATGGGCATCAAAAACTTCATGCCAGCTCTAGAGGAGACCCCATTTCATTC |
| | | | | CTGTACAGCT |
| | | | | GGGCCAAACCCAACCCTAGGGAGGCCACTGTCTCCTAAGCCGTCATCCCCGGGA |
| | | | | TCAGTTTTGGC |
| | | | | TCGTCCATCAGTGATCTGCCATACCACTGTGACTGCATTGAAAGATGTCCCTTTCT |
| | | | | CTCTCTGCCA |
| | | | | GTCGGTCGGTGTGGGACAAAACACAGATATACAGCAGATAGCGGCCAAAAACTT |
| | | | | CACAGACACC |
| | | | | TCTCTCATGTACCCAGAGGACACTTGTAAATCTGGACCAAGTACGAATACACAGA |
| | | | | GTGGTCTTTTC |
| | | | | AACACTCCTCCCCCTACTCCACCGGACCTCAACCAGGACTTCAGTGGATTTCAGCT |
| | | | | TCTAGTGGAT |
| | | | | GTTGCACTCAAACGGGCTGCAGAGATGGAGCTTCAGGCAAAACTTACAGCTTAA |
| | | | | CCCATTTTCAA |
| | | | | GCAAAACAGTTCTCAGAAATGTCATGATTGCCGGGGTGAAGGCAAGAGATGAAT |
| | | | | TGCATTATTTT |
| | | | | ATATATTTTTTATTAATATTTGCACATGGGATTGCTAAAACAGCTTCCTGTTACTG |
| | | | | AGATGTCTTCA |
| | | | | ATGGAATACAGTCATTCCAAGAACTATAAACTTAAAGCTACTGTAGAAACAAAGG |
| | | | | GTTTTCTTTTT |
| | | | | TAAATGTTTCTTGGTAGATTATTCATAATGTGAGATGGTTCCCAATATCATGTGAT |
| | | | | TTTTTTTTCCTC |
| | | | | CCCTTCCCTTTTTTTGTTATTTTTTCAGACTGTGCAATACTTAGAGAACCTATAGCA |
| | | | | TCTTCTCATTC |
| | | | | CCATGTGGAACAGGATGCCCACATACTGTCTAATTAATAAATTTTCCATTTTTTTTC |
| | | | | AAACAA |
| 29087 | THYN1 | HSX2_01718539 | 146 | ATCTCCGTTGATTCCAGAATCGTCCGCACTAAAG |
| 153090 | DAB2IP | HSX2_02252381 | 147 | GTGAGTTCATCAAAGCGCTGTATGAGTCAGATGAGAACTGCGAAGTGGATCCCA |
| | | | | GCAAGTGCTC |
| | | | | GGCCGCTGACCTCCCAGAGCACCAGGGCAACCTCAAGATGTGCTGCGAGCTGGC |
| | | | | CTTCTGCAAG ATCATCAACTCCTACTG |
| 153090 | DAB2IP | HSX2_02252398 | 148 | TGTCTTCCCACGGGAGTTGAAAGAGGTGTTTGCCTCGTGGAGGCAGGAGTGCAG |
| | | | | CAGTCGCGGC |
| | | | | CGCCCGGACATCAGTGAGCGGCTCATCAGCGCCTCCCTCTTCCTGCGCTTCCTCTG |
| | | | | CCCAGCCATC |
| | | | | ATGTCGCCCTCACTCTTCAACCTGCTGCAGGAGTACCCTGATGACCGCACTGCCC |
| | | | | GCACCCTCACC CTCATCGCCAAGGTCACCCAGAACCTGGCCAACTTTGCCAA |
| 153090 | DAB2IP | HSX2_02252492 | 149 | GAGAAGCGCATTGCCTCGTTGGATGCCGCCAATGCCCGCCTCATGAGTGCCCTG |
| | | | | ACCCAGCTGAA AGAGAG |
| 1611 | DAP | HSX2_00873858 | 150 | TGAAAGCTGGTGGAATGCGAATTGTGCAGAAACACCCACATACAG |
| 1611 | DAP | HSX2_00873859 | 151 | GAGACACCAAAGAAGAGAAAGACAAGGATGACCAGGAATGGGAAAGCCCCAG |
| 1717 | DHCR7 | HSX2_00880610 | 152 | GTGGACTGGTTTTCACTGGCGAGCGTCATCTTCCTACTGCTGTTCGCCCCCTT |
| 9249 | DHRS3 | HSX2_01394249 | 153 | GCCTTCCTGCCACGTATGCTGGAGCTGCAGAATGGCCACATCGTGTGCCTCAACT |
| | | | | CCGTGCTGGC |
| | | | | ACTGTCTGCCATCCCCGGTGCCATCGACTACTGCACATCCAAAGCGTCAGCCTTC |
| | | | | GCCTTCATGGA |
| | | | | GAGCCTGACCCTGGGGCTGCTGGACTGTCCGGGAGTCAGCGCCACCACAGTGCT |
| | | | | GCCCTTCCAC ACCAGCACCGAGATGTTCCAGGGCATGAGAGTCAG |
| 9249 | DHRS3 | HSX2_02529585 | 154 | ACCACCAAGGCCTTCCTGCCACGTATGCTGGAGCTGCAGAATGGCCACATCGTGT |
| | | | | GCCTCAACTC |
| | | | | CGTGCTGGCACTGTCTGCCATCCCCGGTGCCATCGACTACTGCACATCCAAAGCG |
| | | | | TCAGCCTTCG |
| | | | | CCTTCATGGAGAGCCTGACCCTGGGGCTGCTGGACTGTCCGGGAGTCAGCGCCA |
| | | | | CCACAGTGCT |
| | | | | GCCCTTCCACACCAGCACCGAGATGTTCCAGGGCATGAGAGTCAG |
| 1730 | DIAPH2 | HSX2_00881579 | 155 | TCTAGATAAACTTTTAGGGGCTATAACAACAGCAGCAGAAAGAAATAACAGGGA |
| | | | | ACGATTTTCAC |
| | | | | CAATTGTGGAAGGTTTAGAAAATCAGGAAGCCTTGCAATTACAG |
| 1756 | DMD | HSX2_00884014 | 156 | GTGAGGAAGATCTTCTCAGTCCTCCCCAGGACACAAGCACAGGGTTAGAGGAGG |
| | | | | TGATGGAGCA ACTCAACAACTCCTTCCCTAGTTCAAGAG |
| 56171 | DNAH7 | HSX2_01892342 | 157 | CACAAAAGCTACATCGAATACACAAAGACTCTGCCACTGACCCCAGCACCAGAAA |
| | | | | TCTTTGGGAT |
| | | | | GAATGCCAATGCAGATATCACTAAGGATCAGTCAGAAACTCAGCTGCTATTTGAT |
| | | | | AACATTCTTCT TACACAG |
| 23348 | DOCK9 | HSX2_01626871 | 158 | GACATTGTTTAAGGATGCATCTGGAAATCTTGACAAAAATGCCAGATTTTCTGCC |
| | | | | ATCTACAGGCA |
| | | | | AGACAGCAATAAGCTATCCAATGATGACATGCTCAAGTTACTTGCAGACTTTCGG |
| | | | | AA |
| 23348 | DOCK9 | HSX2_01627334 | 159 | ATGTCCATTTCAACGAGGATGTGCTGATGGAGCT |
| 23348 | DOCK9 | HSX2_01627375 | 160 | GTCAATGCTGGCCCACTAGCATATGCGCGAGCTTTCTTAGATGATACAAACACAA |
| | | | | AGCGATATCC TGACAATAAAGTGAAGCTGCTTAAGGAAGTTTTCAG |
| 23348 | DOCK9 | HSX2_01627409 | 161 | AACGCCATCAGTGGGACTCCAACAAGCACAATGG |
| 54344 | DPM3 | HSX2_02553775 | 162 | AGTTCGGACCTCTGGGATCGGCGATTCCCCTCTGGCCAGGGCTGGTTTTAATGCT |
| | | | | CTCCGTGGGC |
| | | | | GGGCTTCGGTTGAGTTTGGTCCGCTTTTCCTTTCTGCTCCTCAGGGGAGCATTGCT |
| | | | | TCCTTCTCTC GCA |
| 1809 | DPYSL3 | HSX2_00890973 | 163 | CTGGAAGCTGAGGCTGTGTTCCGTGCCATCACCATTGCCAGCCAAACCAATTGCC |
| | | | | CTCTCTACGTC |
| | | | | ACAAAGGTCATGAGCAAGAGTGCAGCTGACCTCATCTCACAAGCCAGGAAAAAA |
| | | | | G |
| 1837 | DTNA | HSX2_00892824 | 164 | AGGATGAGATCATAGTTTCAGGATCCTCAGGAAACCCTGGTACTGGCAGCAGCC |
| | | | | AGCCTCTGCTGTGCCCACATGACCCACAACTCTGGC |
| 23220 | DTX4 | HSX2_01610552 | 165 | GAATCACTGGGATCCTCATGAGTGCAGCGGGGCTGCCTGTGTGTCTCACCAGGC |
| | | | | CACCAAAGCT |
| | | | | GGTCCTACACCCACCCCCCGTCAGCAAGAGTGAAATAAAATCCATCCCAGGGGTT |
| | | | | TCCAACACAA GCCGCAAGACCACCAAAAAACAAGCCAAGAAAG |
| 53905 | DUOX1 | HSX2_01787484 | 166 | GTGCTGGACATCAACCAGGCCGACGCAGGGACCCTGCCCCTGGACTCCTCCCAG |
| | | | | AAGGTGCGGG |
| | | | | AGGCCCTGACCTGTGAGCTGAGCAGGGCCGAGTTTGCCGAGTCCCTGGGCCTCA |
| | | | | AGCCCCAGGA |
| | | | | CATGTTTGTGGAGTCCATGTTCTCTCTGGCTGACAAGGATGGCAATGGCTACCTG |
| | | | | TCCTTCCGAGAGTTCCTGGACATCCTGGTGGTCTTCATGAAAG |
| 1846 | DUSP4 | HSX2_00893757 | 167 | GGCTCAAGAGATCCTCCCACCTCGGCCTCCTGAGTAGCTGGGACTGCAGGTGTG |
| | | | | TGTCATCATGA |
| | | | | CCAATGTGAATTGCTTTTGAAGCTGGTTCATGGGCATGTAGGCCACCGAAGCAAT |
| | | | | TTTAGACCAC |
| | | | | AGTAAGTCAAGCTTTTTTCCCTCCGATGATCACTGGGTGGTTGCAGCATTTTTTGC |
| | | | | ATAAACCTGC |
| | | | | CTAAGACTTGTCTATCGTCTGTGATCAATATGCCATATTACACTAAGGTGCTCCTG |
| | | | | GAAAATTGGG |
| | | | | TGCAGTTCAAATTTTCCTACAGCAAATCATTTGGCAAGGCCAGCCATTGGGGAAA |
| | | | | CCAGACAACT |
| | | | | AGAGATAACCCTGAAATGAATCCTTTTGTAAATTGAAGCACCATCTTTTCTTTTTTT |
| | | | | GCATAAATTG |
| | | | | GAGGTTTTAATTTTAGGGCAGTTACCTGAAGTGAAATATACCAACAATTTCTTGT |
| | | | | GTTCTTTAAATT |
| | | | | CCTAGTTAGGTGAATATTTTTGAAGGTCCTCTTTTGAATAAAGAGGGGAATGGAC |
| | | | | ACCACATTTCA |
| | | | | GGTCTTCTCGAAGTGTGGAAGGGCAAGAGAGCATCAGTGAGCTGATGGTGGAT |
| | | | | TGCTTACATCG |
| | | | | GATTCCATTGGTATGAATTTCCCAAACTGGAAATCAAAGCGCCAGGGTGGGGTT |
| | | | | GGGGCTGACT |
| | | | | GCTGGTGAGGGGGCTGGCCGCTGGCTCCCGTGACGTGCGTCATGGGCACGCAG |
| | | | | GCGCCATTTTG |
| | | | | AATCTATCGTCGGCACGTGGGTGCCATTTTGAATCCTTAGTTGGGCCTTTCTAAAT |
| | | | | GGAGAATGG |
| | | | | CTTTGGAGGGAGACACGTTTTCTGTGGGGAGGGTTTGGGGGGGAGGGAGGAG |
| | | | | GGAACAAGCTA |
| | | | | CATGCTATTTTGTTTGTAGTATTGTGGAACAGTCTTGTTATGGAGTGCCAGCTTAG |
| | | | | AGGTTGTTGC |
| | | | | AAACTTGTCTAGAAGTGAGAGCATGGTTTTTTTTAGCCCTTTGAGAGTCTACATCT |
| | | | | AATGAACATT |
| | | | | CTTGCTCACCCATAAATAACGTCAAGCCTCAATGTCACCGTCACGTTGGGATACTC |
| | | | | TTTCTCATCT |
| | | | | GGCATCCTAGACAGGACAAGGTTGGTTACCTTTCCTTCCATGAACCATGAACCTG |
| | | | | TGACGGCATC |
| | | | | ATTCATCCTGACTTCACCAAGCTCCGCCTGTGGGTGAGGCCAGAGCTCCCACTGG |
| | | | | CAATTTTTAG |
| | | | | AAGAGCCAGAGGCTCCCTGCTTCCTCTAGAAATAACAGTTCAGGGTGAAGCATG |
| | | | | GAGGGTTTCA |
| | | | | GTTCCCAGACAATGGAACCATTTAGAGACAACACAGTTGGACATTTCCACTTTTTC |
| | | | | CTTGATTCCT |
| | | | | GGAAGTCCAGTGGGTTCTGCAGCTGAAAAAGCCCTGGGTCCCAGCAGCAGAGA |
| | | | | GACAGGACAG |
| | | | | AGGGGATGCTTGGGCGGGGAGGGACGGTAACCTGCAGAACAGATTCCATTTTTA |
| | | | | TAGAACGAGT |
| | | | | ACACGTTTGCTAAAACAGTCCTGCTTTCCCAGACTGGATTCCCACCACAGGGACA |
| | | | | GTCGGAACTC |
| | | | | AGGACTAGCTCCAGCGACATCTTTCCTCCGAATTCAAGCCTTCTATCACAATGTCA |
| | | | | AAACAGCTAT |
| | | | | TTATAAAGCCATTTTCATTGTACTTGATAACAGCACGAGTCCCAAAACTTTTAGAA |
| | | | | ATAAAATAGG |
| | | | | ACATTGGCTTGATTGAAAAGAGGGACTTTTTAAAAATTGTTCTTTCGTCAGAAGC |
| | | | | CTTTTGGATGA |
| | | | | CTTACAATAGCTCTGATGAAGATACCACCCCAGCGTCAGTCCAATAGGTCAGTGA |
| | | | | GTTTCAACAG |
| | | | | GCATCCATCCCTCCCATGAAGGGATTCTGGTGATGGGAAGTTTCTGTAATGACAG |
| | | | | GAAAGCATTG |
| | | | | ACCCTCATTGATTGTCAACTTTGGTATTAGCCATGAAAGACAGGATGCTCATTGG |
| | | | | GTGTTCTGTAG |
| | | | | AGTGAGGAATGCTGCCTATTCCCTCCCAGAACGTCTGACCCAGGGGTGTGTGTTG |
| | | | | AGGAGCCCT |
| | | | | GGGGGAAATGGACCAAGTTTTCCCACAGAGCAGTATTAGGCTGAAGAGCAGGT |
| | | | | GACTGGTAGG |
| | | | | CCCCAGCTCCCATCATTCCCTCCCAAAGCCATTTTGTTCAGTTGCTCATCCACGCTG |
| | | | | GATTCCAGA |
| | | | | GAGTTTTCCAATTTGGGAAGCCATGAGAAAGGTTTTTAAATCTTGGGAAGATGG |
| | | | | AGAGAGGGAC |
| | | | | ATAGGATAGTTGACTCCAACATGACAGGAAGAGGCTGGAGATTGGGAATTGGC |
| | | | | CATCAACCAAG |
| 1877 | E4F1 | HSX2_00894999 | 168 | CCCCCCGTCTCCCAGGAGCTCCCCTGCTCCAGCGAGGGCAGCCGTGAGAACCTG |
| | | | | CTGCACCAG |
| 1889 | ECE1 | HSX2_00895217 | 169 | ACCCCAACGGCCTGCAGGTGAACTTCCACAGCCC |
| 1889 | ECE1 | HSX2_00895219 | 170 | GATCCCCCTCTGTGTGCCTGAGCGAAGCTTGTGTCTCAGTGACCAGCTCCATCTT |
| | | | | GAGCTCCATG |
| | | | | GACCCCACAGTGGACCCCTGCCATGACTTCTTCAGCTACGCCTGTGGGGGCTGGA |
| | | | | TCAAGGCCAA |
| | | | | CCCAGTCCCTGATGGCCACTCACGCTGGGGGACCTTCAGCAACCTCTGGGAACAC |
| | | | | AACCAAGCA ATCATCAAGCACCTCCTCG |
| 1889 | ECE1 | HSX2_00895231 | 171 | CTCGGGGGCTGGAACATCACAGGTCCCTGGGCCAAGGACAACTTCCAGGACACC |
| | | | | CTGCAGGTGG |
| | | | | TCACCGCCCACTACCGCACCTCACCCTTCTTCTCTGTCTATGTCAGTGCCGATTCCA |
| | | | | AGAACTCCAA CAGCAACGTGATCCAG |
| 1889 | ECE1 | HSX2_00895252 | 172 | ACAACTACATGATCTGGAACCTGGTGCGGAAAACAAGCTCCTTCCTTGACCAGCG |
| | | | | CTTTCAGGAC GCCGATGAGAAGTTCATGGAAGTCATGTACGGGACCAAGAAG |
| 1889 | ECE1 | HSX2_00895278 | 173 | GTGGAGCATGACCCCGCCCATGGTGAACGCCTACTACTCGCCCACCAAGAATGA |
| | | | | GATTGTGTTTC |
| | | | | CGGCCGGGATCCTGCAGGCACCATTCTACACACGCTCCTCACCCAA |
| 1889 | ECE1 | HSX2_00895288 | 174 | GACGGGAGTATGACAAGGACGGGAACCTCCGGCCATGGTGGAAGAACTCATCC |
| | | | | GTGGAGGCCT |
| | | | | TCAAGCGTCAGACCGAGTGCATGGTAGAGCAGTACAGCAACTACAGCGTGAACG |
| | | | | GGGAGCCGG |
| | | | | TGAACGGGCGGCACACCCTGGGGGAGAACATCGCCGACAACGGGGGTCTCAAG |
| | | | | GCGGCCTATC GG |
| 1893 | ECM1 | HSX2_00895741 | 175 | TGGGAGGATACCCTTGACAAATACTGTGACCGGGAGTATGCTGTGAAGACCCAC |
| | | | | CACCACTTGTG |
| | | | | TTGCCGCCACCCTCCCAGCCCTACTCGGGATGAGTGCTTTGCCCGTCGGGCTCCTT |
| | | | | ACCCCAACTA |
| | | | | TGACCGGGACATCTTGACCATTGACATCGGTCGAGTCACCCCCAACCTCATGGGC |
| | | | | CACCTCTGTGGAAACCAAAGAGTTCTCACCAAGCA |
| 2202 | EFEMP1 | HSX2_00921723 | 176 | ACAGCCCAGATTATTGTCAATAATGAACAGCCTCAG |
| 1958 | EGR1 | HSX2_00900683 | 177 | AGTCTTTTCCTGACATCTCTCTGAACAACGAGAAGGTGCTGGTGGAGACCAGTTA |
| | | | | CCCCA |
| 1958 | EGR1 | HSX2_00900684 | 178 | GCCAAACCACTCGACTGCCCCCCATCACCTATACTGGCCGCTTTTCCCTGGAGCCT |
| | | | | GCACCCAACA |
| 1958 | EGR1 | HSX2_00900686 | 179 | GCCTCCGCCTCCCAGAGCCCACCCCTGAGCTGCGCAGTGCCATCCAACGACAGCA |
| | | | | GTCCCATTTA CTCAG |
| 1958 | EGR1 | HSX2_00900687 | 180 | CGGCACCCACCTTCCCCACGCCGAACACTGACATTTTC |
| 1958 | EGR1 | HSX2_00900688 | 181 | CCTGAGCCACAAAGCCAGGCCTTCCCGGGCTCGGCAGGGACAGCGCTCCAGTAC |
| | | | | CCGCCTCCTG |
| | | | | CCTACCCTGCCGCCAAGGGTGGCTTCCAGGTTCCCATGATCCCCGACTACCTGTTT |
| | | | | CCACAGCAG |
| | | | | CAGGGGGATCTGGGCCTGGGCACCCCAGACCAGAAGCCCTTCCAGGGCCTGGA |
| | | | | GAGCCGCACC |
| | | | | CAGCAGCCTTCGCTAACCCCTCTGTCTACTATTAAGGCCTTTGCCACTCAG |
| 1958 | EGR1 | HSX2_00900689 | 182 | TCGGGCTCCCAGGACCTGAAGGCCCTCAATACCAGCTACCAGTCCCAGCTCATCA |
| | | | | AACCCAGCCG |
| | | | | CATGCGCAAGTACCCCAACCGGCCCAGCAAGACGCCCCCCCACGAACGCCCTTAC |
| | | | | GCTTGCCCAG |
| | | | | TGGAGTCCTGTGATCGCCGCTTCTCCCGCTCCGACGAGCTCACCCGCCACATCCG |
| | | | | CATCCACACA |
| | | | | GGCCAGAAGCCCTTCCAGTGCCGCATCTGCATGCGCAACTTCAGCCGCAGCGAC |
| | | | | CACCTCACCAC |
| | | | | CCACATCCGCACCCACACAGGCGAAAAGCCCTTCGCCTGCGACATCTGTGGAAG |
| | | | | AAAGTTTGCCA |
| | | | | GGAGCGATGAACGCAAGAGGCATACCAAGATCCACTTGCGGCAGAAGGACAAG |
| | | | | AAAGCAGACA |
| | | | | AAAGTGTTGTGGCCTCTTCGGCCACCTCCTCTCTCTCTTCCTACCCGTCCCCGGTT |
| | | | | GCTACCTCTTA |
| | | | | CCCGTCCCCGGTTACTACCTCTTATCCATCCCCGGCCACCACCTCATACCCATCCCC |
| | | | | TGTGCCCACC |
| | | | | TCCTTCTCCTCTCCCGGCTCCTCGACCTACCCATCCCCTGTGCACA |
| 1958 | EGR1 | HSX2_00900692 | 183 | AAAAAAAAAAGAAAAGCCAAGCAAACCAATGGTGATCCTCTATTTTGTGATGAT |
| | | | | GCTGTGACAAT |
| | | | | AAGTTTGAACCTTTTTTTTTGAAACAGCAGTCCCAGTATTCTCAGAGCATGTGTCA |
| | | | | GAGTGTTGTT |
| | | | | CCGTTAACCTTTTTGTAAATACTGCTTGACCGTACTCTCACATGTGGCAAAATATG |
| | | | | GTTTGGTTTTT |
| | | | | CTTTTTTTTTTTTTTTGAAAGTGTTTTTTCTTCGTCCTTTTGGTTTAAAAAGTTTCAC |
| | | | | GTCTTGGTGCC |
| | | | | TTTTGTGTGATGCGCCTTGCTGATGGCTTGACATGTGCAATTGTGAGGGACATGC |
| | | | | TCACCTCTAGC |
| | | | | CTTAAGGGGGGCAGGGAGTGATGATTTGGGGGAGGCTTTGGGAGCAAAATAAG |
| | | | | GAAGAGGGCT |
| | | | | GAGCTGAGCTTCGGTTCTCCAGAATGTAAGAAAACAAAATCTAAAACAAAATCTG |
| | | | | AACTCTCAAA |
| | | | | AGTCTATTTTTTTAACTGAAAATGTAAATTTATAAATATATTCAGGAGTTGGAATG |
| | | | | TTGTAGTTACC |
| | | | | TACTGAGTAGGCGGCGATTTTTGTATGTTATGAACATGCAGTTCATTATTTTGTG |
| | | | | GTTCTATTTTAC |
| | | | | TTTGTACTTGTGTTTGCTTAAACAAAGTGACTGTTTGGCTTATAAACACATTGAAT |
| | | | | GCGCTTTATTG |
| | | | | CCCATGGGATATGTGGTGTATATCCTTCCAAAAAATTAAAACGAAAATAAAGTA |
| | | | | GCTGCGATTGG G |
| 1967 | EIF2B1 | HSX2_00901053 | 184 | TAAGAAAATGGCCAAAGCCCTCTGCCACCTCAACGTCCCTGTCACTGTGGTGCTA |
| | | | | GATGCTGCT |
| 55740 | ENAH | HSX2_01871605 | 185 | TGAACAGAGTATCTGTCAGGCAAGAGCTGCTGTGATGGTTTATGATGATGCCAA |
| | | | | TAAGAAGTGG |
| | | | | GTGCCAGCTGGTGGCTCAACTGGATTCAGCAGAGTTCATATCTATCACCATACAG |
| | | | | GCAAC |
| 55740 | ENAH | HSX2_01871707 | 186 | CAATCAGGCAGGAACTGAGCAAGTCAAATACTGCATAGAGGAACAGACTAAGG |
| | | | | AGAGATAGGA |
| | | | | CTTTAATCTGGAGGAAAAATATCCTACAAACAACAACTGTTCACAACAGCAAACC |
| | | | | CCTACATTTAT |
| | | | | GAGCTGTAAGAAGAAAATGGAGACAAACAGAAGGAGGGAAAAACCAACCTACT |
| | | | | CTGAAAGCCTT |
| | | | | CAGACATTATGACTCTGGTGATAAGCTCTTTCCCTCTCCGTTTGCTGCTTTTTTCTG |
| | | | | GCCTTTACAA |
| | | | | CAGAATGGAAGAGAATCATTTAAGAGTTCCTGTAACAGTTATGCAGAAAATACTA |
| | | | | AAACCCATCA |
| | | | | GGCAAGATCACCACGCATTGAAATATTTTCATATCAAGATAAAGTCGCACATTTT |
| | | | | CCACAATACAT |
| | | | | TGCTAAAATAAAGAGGAGAAAGGCTTAGGAAGTTTTTTTGCAGAGAGTGCTGGT |
| | | | | AAAGAATTGA |
| | | | | GCAAGTTTGCTATTGTATTGTAATGTTTCTCTCAGGTTTGTTCTTCCTATCATGTTT |
| | | | | GATATTCCATG |
| | | | | AATAATTGAGATCAGCCCTATGTAAGTTAAGATCATAATATGTGGAACAAATGGA |
| | | | | ATTGTAAGTG |
| | | | | CTTTCAAAGGGTAATATTTATAAGAAAGTGTCCGAAAAATGTTTCTTCAGCTTGA |
| | | | | GAAATTTTAGA |
| | | | | ATGATAGGAAGTTTCTCGAGTTAGCCTTCATGCAATTTTGTAGATTAAAACATAA |
| | | | | AATTTGTCCAG AACTTAA |
| 55740 | ENAH | HSX2_01871807 | 187 | TATGATTCATTACACAGACCAAAATCCACACCCT |
| 55740 | ENAH | HSX2_01871851 | 188 | TCACCTGTTATCTCCAGACCAAAATCCACACCCT |
| 23052 | ENDOD1 | HSX2_01590519 | 189 | ATCGATGACCCCAACAGCAACCTTGAGGAGGCGATTAATGAGGCAGAGGCCATC |
| | | | | ACCTCTGTGA |
| | | | | ACAGCCTGGGAAGCAAGCAAGCCTTGAATACAGATTACCTTGATTCTGATTACCA |
| | | | | AAGAGGACAG |
| | | | | CTTTACCCATTCTCCCTTAGCAGTGATGTCCAGGTGGCCACATTTACTCTCACAAA |
| | | | | TTCAGCCCCAA |
| | | | | TGACTCAGTCCTTCCAGGAACGGTGGTATGTGAATCTCCACAGCCTAATGGACCG |
| | | | | GGCTTTGACC |
| | | | | CCACAGTGTGGCAGTGGGGAAGACCTATATATCCTCACAGGCACAGTGCCCTCA |
| | | | | GACTACAGAG |
| | | | | TTAAAGACAAAGTGGCAGTCCCTGAGTTTGTTTGGCTGGCAGCCTGTTGTGCTGT |
| | | | | CCCTGGAGGA |
| | | | | GGCTGGGCCATGGGCTTTGTCAAGCACACCCGGGACAGTGACATCATAGAAGAT |
| | | | | GTGATGGTAA |
| | | | | AAGATCTTCAGAAACTGCTTCCATTTAACCCTCAGCTGTTTCAGAACAACTGTGGT |
| | | | | GAAACTGAGC |
| | | | | AAGACACAGAGAAAATGAAAAAAATCCTGGAAGTGGTTAACCAAATCCAGGATG |
| | | | | AAGAACGAAT |
| | | | | GGTACAATCTCAAAAGAGTTCTAGTCCCCTTTCTAGCACCAGGAGCAAGAGGTCT |
| | | | | ACTCTGTTGC |
| | | | | CTCCAGAGGCATCTGAGGGAAGTAGTAGCTTTTTGGGAAAACTCATGGGCTTCA |
| | | | | TTGCTACCCCA |
| | | | | TTCATCAAGCTTTTTCAATTAATTTATTACCTTGTGGTAGCAATCCTGAAGAACATT |
| | | | | GTCTATTTCCT |
| | | | | GTGGTGTGTTACCAAGCAGGTGATTAATGGCATAGAAAGTTGCCTTTACCGCCTG |
| | | | | GGCTCAGCCA |
| | | | | CCATCTCATACTTCATGGCCATTGGGGAAGAGTTGGTGAGCATTCCCTGGAAGGT |
| | | | | GCTCAAGGTC |
| | | | | GTGGCCAAAGTCATCAGGGCTCTCCTCCGGATCCTTTGTTGTCTGCTGAAGGCCA |
| | | | | TTTGCCGAGTT |
| | | | | CTGAGCATCCCTGTCCGTGTCCTTGTGGATGTGGCCACTTTCCCTGTGTACACCAT |
| | | | | GGGCGCTATT |
| | | | | CCAATTGTTTGCAAGGACATTGCACTGGGCCTTGGTGGCACTGTCTCACTGCTCT |
| | | | | TTGACACTGCT |
| | | | | TTTGGTACCCTGGGTGGCCTATTTCAGGTGGTTTTTAGTGTCTGCAAGCGGATTG |
| | | | | GCTACAAGGT |
| | | | | TACTTTTGACAATTCTGGGGAGTTATAAACTCAAAAAACTAATAGTATCCAGTCA |
| | | | | CAGTGAATTTG |
| | | | | AAAGCTGGAATAGTTTGTCTTTACAATGGGTTTCTGTTCACTGTCAGTTATCATTA |
| | | | | TATTTTGGCCT |
| | | | | TTGGTGGGGATGTCTGCTTGTTTTTGCAAAAGAAGATGGCAGAATTTAGACTTGA |
| | | | | CAGAGGAGA |
| | | | | AATGCTCAGGGTGAGATTAGGTGTAGTAATCTGCTGTTTACCTCCAGTTATATGT |
| | | | | GCAAACTCCCA |
| | | | | AGCCACTAATAACTTCAGTTATGCACTCTAACACAGACGACCACCTGAAATGCAC |
| | | | | TGGTATTTATT |
| | | | | TCTGATAATTAAAAATTACAGGGGAGGGAAGAACTAGAAAAAGAACAACTTTAG |
| | | | | ACCAAAGGTG |
| | | | | TCTGAGAAAAGGAGAAAGGGAGCTTGTTCTTCCCATTGCTCTTTGTGATTTAAGG |
| | | | | CAAAACAGAT TAAAAAAAAAATCTGCAGC |
| 953 | ENTPD1 | HSX2_00825916 | 190 | TTTGCTCCAAGAATATCCTAGCCATCCTTGGCTTCTCCTCTATCATAGCTGTGATA |
| | | | | GCTTTGCTTGC TGTGGGGTTGACCCAGAACAAAGCATTGCCAGAAAACGTTAAG |
| 953 | ENTPD1 | HSX2_00825924 | 191 | TGCCAGAAAACGTTAAGTATGGGATTGTGCTGGA |
| 953 | ENTPD1 | HSX2_00825926 | 192 | GTCCTGGAATCTCAAAATTTGTTCAGAAAGTAAATGAAATAGGCATTTACCTGAC |
| | | | | TGATTGCATGG |
| | | | | AAAGAGCTAGGGAAGTGATTCCAAGGTCCCAGCACCAAGAGACACCCGTTTACC |
| | | | | TGGGAGCCAC GGCAGGCATGCGGTTG |
| 953 | ENTPD1 | HSX2_00825931 | 193 | AGAATGCAGGGTTAAAGGTCCTGGAATCTCAAAA |
| 953 | ENTPD1 | HSX2_00825934 | 194 | GGTGCCAGGATCATTACTGGCCAAGAGGAAGGTGCCTATGGCTGGATTACTATC |
| | | | | AACTATCTGCT GGGCAAATTCAGTCAG |
| 953 | ENTPD1 | HSX2_00825945 | 195 | GTTGCAAGTAATGAAATTCTCAGGGACCCATGCTTTCATCCTGGATATAAGAAGG |
| | | | | TAGTGAACGT |
| | | | | AAGTGACCTTTACAAGACCCCCTGCACCAAGAGATTTGAGATGACTCTTCCATTCC |
| | | | | AG |
| 953 | ENTPD1 | HSX2_00825947 | 196 | TTTGAAATCCAGGGTATTGGAAACTATCAACAATGCCATCAAAGCATCCTGGAGC |
| | | | | TCTTCAACACC |
| | | | | AGTTACTGCCCTTACTCCCAGTGTGCCTTCAATGGGATTTTCTTGCCACCACTCCA |
| | | | | GGGGGATTTT GGG |
| 953 | ENTPD1 | HSX2_00825952 | 197 | TGGCCAAGGACATTCAGGTTGCAAGTAATGAAAT |
| 953 | ENTPD1 | HSX2_00825954 | 198 | GCATTTTCAGCTTTTTACTTTGTGATGAAGTTTTTAAACTTGACATCAGAGAAAGT |
| | | | | CTCTCAGGAA |
| | | | | AAGGTGACTGAGATGATGAAAAAGTTCTGTGCTCAGCCTTGGGAGGAG |
| 953 | ENTPD1 | HSX2_00825959 | 199 | ATAAAAACATCTTACGCTGGAGTAAAGGAGAAGTACCTGAGTGAATACTGCTTTT |
| | | | | CTGGTACCTA |
| | | | | CATTCTCTCCCTCCTTCTGCAAGGCTATCATTTCACAGCTGATTCCTGGGAGCACA |
| | | | | TCCATTTCATT GGCAAG |
| 953 | ENTPD1 | HSX2_00825964 | 200 | ATCCAGGGCAGCGACGCCGGCTGGACTTTGGGCTACATGCTGAACCTGACCAAC |
| | | | | ATGATCCCAG |
| | | | | CTGAGCAACCATTGTCCACACCTCTCTCCCACTCCACCTATGTCTTCCTCATGGTTC |
| | | | | TATTCTCCCT |
| | | | | GGTCCTTTTCACAGTGGCCATCATAGGCTTGCTTATCTTTCACAAGCCTTCATATTT |
| | | | | CTGGAAAGAT |
| | | | | ATGGTATAGCAAAAGCAGCTGAAATATGCTGGCTGGAGTGAGGAAAAAAATCGT |
| | | | | CCAGGGAGCA |
| | | | | TTTTCCTCCATCGCAGTGTTCAAGGCCATCCTTCCCTGTCTGCCAGGGCCAGTCTT |
| | | | | GACGAGTGTG |
| | | | | AAGCTTCCTTGGCTTTTACTGAAGCCTTTCTTTTGGAGGTATTCAATATCCTTTGCC |
| | | | | TCAAGGACTT |
| | | | | CGGCAGATACTGTCTCTTTCATGAGTTTTTCCCAGCTACACCTTTCTCCTTTGTACT |
| | | | | TTGTGCTTGTA |
| | | | | TAGGTTTTAAAGACCTGACACCTTTCATAATCTTTGCTTTATAAAAGAACAATATT |
| | | | | GA |
| 953 | ENTPD1 | HSX2_00825979 | 201 | TATGGAAGATACAAAGGAGTCTAACGTGAAGACA |
| 953 | ENTPD1 | HSX2_02495027 | 202 | AGTCTAACGTGAAGACATTTTGCTCCAAGAATATCCTAGCCATCCTTGGCTTCTCC |
| | | | | TCTATCATAGC |
| | | | | TGTGATAGCTTTGCTTGCTGTGGGGTTGACCCAGAACAAAGCATTGCCAGAAAA |
| | | | | CGTTAAG |
| 953 | ENTPD1 | HSX2_02495030 | 203 | GTCCTGGAATCTCAAAATTTGTTCAGAAAGTAAATGAAATAGGCATTTACCTGAC |
| | | | | TGATTGCATGG |
| | | | | AAAGAGCTAGGGAAGTGATTCCAAGGTCCCAGCACCAAGAGACACCCGTTTACC |
| | | | | TGGGAGCCAC GGCAGGCATGCGGTTGCTCAG |
| 953 | ENTPD1 | HSX2_02495034 | 204 | CAGTTTGAAATCCAGGGTATTGGAAACTATCAACAATGCCATCAAAGCATCCTGG |
| | | | | AGCTCTTCAA |
| | | | | CACCAGTTACTGCCCTTACTCCCAGTGTGCCTTCAATGGGATTTTCTTGCCACCAC |
| | | | | TCCAGGGGGA TTTTGGG |
| 954 | ENTPD2 | HSX2_00826105 | 205 | GCCGCGGACACTGCAGTGGGCTGGGCGCTCGGCTACATGCTGAACCTGACCAAC |
| | | | | CTGATCCCCG |
| | | | | CCGACCCGCCGGGGCTGCGCAAGGGCACAGACTTCAGCTCCTGGGTCGTCCTCC |
| | | | | TGCTGCTCTTC |
| | | | | GCCTCCGCGCTCCTGGCTGCGCTTGTCCTGCTGCTGCGTCAGGTGCACTCCGCCA |
| | | | | AGCTGCCAAG |
| | | | | CACCATTTAGGGGCCGACGGGGGCAGCTGCCCCATCCCTCCCCCAACCCCTGTAT |
| | | | | CCCCACCCCG |
| | | | | TACTCCCACCCCTCCCACAACCCCTGTACCTCCCACCCCTGTATCCCCACCCCTCCA |
| | | | | CCCACCCCTC |
| | | | | TCCCAACCTCTCTCCCCGCCCCTGTATCCTGCATTCCTCCACCCACCCTCTATCCCC |
| | | | | CACCGCTCCA |
| | | | | CCCCACCACTGTCTTCTCCATCCTTCCACCCCACCCTCAGCGTCTCTGCCCCTAAGG |
| | | | | CAGCCCAGG |
| | | | | AAATAGGAACTGAGACTCTGGTACCCACAGGAGCCTGGGTGGGCAAAGAGCGC |
| | | | | TCAATCCAGCT |
| | | | | CCTTGAACCCCTCCAGCCCGCTTCAGCCTGGGCATCACTGCAGGCCCCGTGCTCC |
| | | | | TCCTCCTCCTC |
| | | | | CTCAGGGCTGGGTCTCCAGAGAGTGGGGCCTTGGTCCTGAGAATCAGCCCTTAG |
| | | | | AGGCTCCTTCT |
| | | | | GTGTAGTCTGGGTCTGTACTGGGGAGGGTCACAGCCCACGGGCTGGCAGCCAG |
| | | | | CCCAGCACCTA CTTGTAAAAATTTTGTAATAAAAAGTTTTTCCTAGAGACGTG |
| 956 | ENTPD3 | HSX2_00826288 | 206 | AAGAACCTGTGGCACATGTGGGTGCACCCGCATGGAGTGGAAACCACGGGTGC |
| | | | | CCTGGACTTAG |
| | | | | GTGGTGCCTCCACCCAAATATCCTTCGTGGCAGGAGAGAAGATGGATCTGAACA |
| | | | | CCAGCGACAT |
| | | | | CATGCAGGTGTCCCTGTATGGCTACGTATACACGCTCTACACACACAGCTTCCAG |
| | | | | TGCTATGGCC |
| | | | | GGAATGAGGCTGAGAAGAAGTTTCTGGCAATGCTCCTGCAG |
| 956 | ENTPD3 | HSX2_00826298 | 207 | GCTTTTGCAGGATTCTACTACACAGCCAGTGCTTTAAATCTTTCAGGTAGCTTTTC |
| | | | | CCTGGACACCT TCAACTCCAGCACCTGGAATTTCTGCTCACAGAATTGGAGTCAG |
| 79852 | EPHX3 | HSX2_02027236 | 208 | AGCTCGGGCCTGCGTCTGCACTATGTCTCGGCTGGACGAGGTAACGGACCCCTC |
| | | | | ATGCTGTTTCT GCACGGCTTCCCTGAGAACTG |
| 2059 | EPS8 | HSX2_00910337 | 209 | TGGCTACGGATCATCACCTACCTTTTCCCAGACGGACAGAGAACATGGTTCAAAA |
| | | | | ACAAGTGCAA AGGCCCTTTATG |
| 2059 | EPS8 | HSX2_00910352 | 210 | TTGACTACCTTTGTCCTGGATCGGAAAGATGCTATGATCACTGTTGATGATGGAA |
| | | | | TAAGGAAATT |
| | | | | GAAATTGCTTGATGCCAAGGGCAAAGTGTGGACTCAAGATATGATTCTTCAAGT |
| | | | | GGATGACAGA GCTGTGAGCCTGATTGATTTAGAATCAAAG |
| 2059 | EPS8 | HSX2_00910358 | 211 | AATGAACTGGAGAATTTTCCTTTAAACACAATCCAGCACTGCCAAGCTGTGATGC |
| | | | | ATTCATGCAGC |
| | | | | TATGATTCAGTTCTTGCACTGGTGTGCAAAGAGCCAACCCAGAACAAGCCAGATC |
| | | | | TTCATCTCTTC CAGTGTGATGAGGTTAAG |
| 2059 | EPS8 | HSX2_00910368 | 212 | GATGATTTCCAATGCAGACCCTAGTATACCGCCTCCACCCAGAGCTCCTGCCCCT |
| | | | | GCGCCCCCTG |
| | | | | GGACCGTCACCCAGGTGGATGTTAGAAGTCGAGTGGCAGCCTGGTCTGCATGGG |
| | | | | CAGCCGACCA AGGGGACT |
| 2059 | EPS8 | HSX2_00910373 | 213 | TTGAGAAACCAAGGCAGTATCATGAGCAGGAAGAAACACCTGAGATGATGGCA |
| | | | | GCCCGCATTGA CAGAGATGTG |
| 2059 | EPS8 | HSX2_00910388 | 214 | GCCAAACTGAAGTCTCATATTCAGAATCCTAGTGCTGCAGATTTGGTTCACTTTTT |
| | | | | GTTTACTCCAT TAAATATG |
| 2059 | EPS8 | HSX2_00910393 | 215 | GTGGTGCAGGCAACAGGAGGTCCTGAACTAGCCAGTTCAGTACTTAGTCCCCTAT |
| | | | | TGAATAAGGA |
| | | | | CACAATTGATTTCTTAAATTATACTGTCAATGGTGATGAACGGCAGCTGTGGATG |
| | | | | TCATTGGGAG GAACTTGGATGAAAGCCAG |
| 2059 | EPS8 | HSX2_00910398 | 216 | AGCAGAGTGGCCAAAAGAACAGTTTATTCCACCATATGTTCCACGATTCCGCAAT |
| | | | | GGCTGGGAGC |
| | | | | CCCCAATGCTGAACTTTATGGGAGCCACAATGGAACAAGATCTTTATCAACTGGC |
| | | | | AGAATCTGTG |
| | | | | GCAAATGTAGCAGAACATCAGCGCAAACAGGAAATAAAAAGATTATCCACAGAG |
| 2059 | EPS8 | HSX2_00910403 | 217 | CATTCCAGTGTATCAGAGTATCATCCAGCCGATGGCTATGCGTTCAGTAGCAACA |
| | | | | TTTACACAAG |
| | | | | AGGATCCCACCTGGACCAAGGGGAAGCTGCTGTTGCTTTTAAGCCAACTTCTAAT |
| | | | | CGCCATATAG ATAG |
| 2059 | EPS8 | HSX2_00910408 | 218 | AAATTATGAACCACTCAAAACACAACCCAAGAAATATGCCAAATCCAAGTATGAC |
| | | | | TTTGTAGCAA GGAACAACAGTGAGCTCTCGGTTCTAAAGGATGATATTTTAGAG |
| 2059 | EPS8 | HSX2_00910413 | 219 | ATACTTGATGATCGGAAGCAATGGTGGAAAGTTCGAAATGCAAGTGGAGACTCT |
| | | | | GGATTTGTGC |
| | | | | CAAATAACATTTTGGATATTGTGAGACCTCCAGAATCTGGATTGGGGCGTGCTGA |
| | | | | TCCACCTTATA CTCATACTATACAG |
| 2059 | EPS8 | HSX2_00910418 | 220 | AAACAAAGGATGGAGTATGGCCCAAGACCAGCTGATACTCCCCCTGCTCCATCAC |
| | | | | CTCCTCCAAC |
| | | | | ACCAGCTCCTGTTCCTGTTCCCCTTCCCCCTTCCACTCCAGCACCTGTTCCTGTGTC |
| | | | | AAAGGTCCCA |
| | | | | GCAAATATAACACGTCAAAACAGCAGCTCCAGTGACAGTGGTGGCAGTATCGTG |
| | | | | CGAGACAGCC AGAGACACAAACAACTTCCGGTGGACC |
| 2059 | EPS8 | HSX2_00910423 | 221 | GAAGGAAATCTCAGATGGAGGAAGTGCAAGATGAACTCATCCACAGACTGACCA |
| | | | | TTGGTCGGAG |
| | | | | TGCCGCTCAGAAGAAATTCCATGTGCCACGGCAGAACGTGCCAGTTATCAATATC |
| | | | | ACTTACGACT |
| | | | | CCACACCAGAGGATGTGAAGACGTGGTTACAGTCAAAGGGATTCAACCCTGT |
| 2059 | EPS8 | HSX2_00910433 | 222 | CCCATGCAAACCTGAGTGAGCACAGGATTTGCTTCTAGGCCCATTATTTTTATTAA |
| | | | | AACTGAAAAA |
| | | | | ATTTAAACTGAATTTTTTGACCTTGGAAAATATTTTTCTTACTTTACCAAGGTGAA |
| | | | | GTTTCCTTAATT |
| | | | | AGACTAATTATTTTATCCCCATCCCAGGGTATAAACAGGAATTGTTTTGATAGTG |
| | | | | GTGGAGTTATT |
| | | | | CACTGCAACAAAGCAACAATGTTGTCCATGATTCAAAATCTAAGCAGTTTCGATTT |
| | | | | TGCCTGTGAA |
| | | | | TATGGTGTCTGTCATTCAGGGCATAGCTCACTGTAGGCTAGCCTCTGCTTACTTAA |
| | | | | GTCTCTTCTCT |
| | | | | GACATACTCAATGGAAGAATATTTAGATTTATTTAAAGTTCTTAATGCCAACAGTT |
| | | | | TAAAAAAAAA |
| | | | | TTAAAACATTTGAATGAACTGTAAAGTACAGCCATACCTTGGACATGCAAATATA |
| | | | | AATCTATGGAG |
| | | | | CATTCTCAAGAGAGTTTGTCATGGCTCTGTTGATTGCAACTCCTTGTATAGCTTGT |
| | | | | ATTTTGATTTA |
| | | | | GTTTATATTCTGCTTATTATGTATACTGTGTTCTTATATATGAGAAAGCACAAATG |
| | | | | CGAAAGAGGT |
| | | | | CATGTCTTCTCAAAATCTAGCAAAGGAAGTAGTCTGCATTGGTGTGCATTACAGT |
| | | | | ATTTTGCTTAA |
| | | | | TGAAAGCCTCAGTTCTGAATGTTGATATGAGTAGTTAAAAGGAAGTGGGGCCAT |
| | | | | TTTATGTGTTT |
| | | | | ATCTGTGTCAAGTATTTCTGGTAATAAGAAGCACTTAATTTACACATATTTTAATC |
| | | | | CTGTGAAAGAT |
| | | | | TCCACATAGAGAAAAGAAAGATACCTAACCTTCAACAAATGTTATTTTTGGAAAC |
| | | | | ACAATTTTTGT |
| | | | | CATTAAATGTTATATTATTTCACATATATAAAACAGATGTTATGTAAGAATGTTGT |
| | | | | ATATTTTAACA |
| | | | | TAAATCATTTAGAGAAATTATCTAGATTCATTAATTTTCATAGTGCCTTTTTCACAT |
| | | | | GAGTCAGCTG |
| | | | | GAAAGTCTGCAATAAACAGTATTTGCTGTCTGTTAAGATAGCAGTGGCAGCTCCG |
| | | | | AGTTACAAGA |
| | | | | AATTATGAGAAGACGACAGGAAAAAATCAGTGCTGCCGCTAGTGATTCAGGAGT |
| | | | | GGAATCTTTT |
| | | | | GATGAAGGAAGCAGTCACTAATTTGTTTGTTTGTATTTAAACTCCATTGTTTTTGG |
| | | | | CATTATTCCAA |
| | | | | CATGCTTTGTTTTAAGAAGCCTTGAAGGGAATGTCAGATTCATTTTTCTTGATGTA |
| | | | | ATTTATCACCA TAAAAAAAAA |
| 2059 | EPS8 | HSX2_02499999 | 223 | CACTTGACTACCTTTGTCCTGGATCGGAAAGATGCTATGATCACTGTTGATGATG |
| | | | | GAATAAGGAA |
| | | | | ATTGAAATTGCTTGATGCCAAGGGCAAAGTGTGGACTCAAGATATGATTCTTCAA |
| | | | | GTGGATGACA GAGCTGTGAGCCTGATTGATTTAGAATCAAAG |
| 2065 | ERBB3 | HSX2_00910994 | 224 | AGATACACACCTCAAAGGTACTCCCTCCTCCCGG |
| 7224 | TRPC5 | HSX2_01280361 | 225 | TTTGTTGCTCAGCCCAACTGCCAACAGTTGCTTGCCACCCTGTGGTATGATGGCTT |
| | | | | CCCTGGATGG |
| | | | | CGGCGGAAACACTGGGTAGTCAAGCTTCTAACCTGCATGACCATTGGGTTCCTGT |
| | | | | TTCCCATGCT |
| | | | | GTCTATAGCCTACCTGATCTCACCCAGGAGCAACCTTGGGCTGTTCATCAAGAAA |
| | | | | CCCTTTATCAA |
| | | | | GTTTATCTGCCACACAGCATCCTATTTGACCTTCCTCTTTATGCTTCTCCTGGCTTC |
| | | | | TCAGCACATTG |
| | | | | TCAGGACAGACCTTCATGTACAGGGGCCTCCCCCAACTGTCGTGGAATGGATGA |
| | | | | TATTGCCTTGG GTTCTAG |
| 7224 | TRPC5 | HSX2_01280366 | 226 | GTTTCATTTGGGGTGAGATTAAGGAAATGTGGGATGGTGGATTTACTGAATACA |
| | | | | TCCATGACTGG |
| | | | | TGGAACCTGATGGATTTTGCAATGAACTCCCTCTACCTGGCAACTATTTCCCTGAA |
| | | | | GATTGTGGCC TATGTCAAG |
| 7224 | TRPC5 | HSX2_01280397 | 227 | TGACCATCCTCAGAATCTCATGCCATTTTCCCGCAGAGCTGAGGGATGTGGTCAG |
| | | | | CAGTCCCCAC |
| | | | | CAGCTATCAGATAAGGCAAACTACCCTGCCCATCTTTGTTGCCATTCAGTGCTACG |
| | | | | CGCTGCCCAT |
| | | | | GCTGGGTCAATGAACAGCATCAAAGGGAGGCTGAAGAGATTTCAAAGAATTGTC |
| | | | | AGCTTGTGGT |
| | | | | TTCTGGGTATAAGTGGCTTGTTTCGTTCTTAAATACTACAACTTCTTATTGTGGGG |
| | | | | GCTATTGAGTT |
| | | | | TTCTTGTTTCTTAAAGATGTGTTAGTTTCCTAATGTGTGCCAATGAAATATTTACCC |
| | | | | CATAGTATAT |
| | | | | GTCATATCTAATAAGTTAGGCGAATTATTTTTCTGTTTGTGTGTTTCAGTTAACTTA |
| | | | | CTACATTTGA |
| | | | | AAATTTGATAGTATCAAAATTAGTATTTAGAATTTGTGCTATTTTTTAAAAACATC |
| | | | | TACTACACTCT |
| | | | | AACCAATAGTCACTACATCTCATACATTGGCAAGATAAATAGTTGTCATATTTGTT |
| | | | | GGGTCCTATTC |
| | | | | TACTGTTGATAGTTTTAATTATCATTGCTATCAAATAATTTCTAAGATAAAAGCCT |
| | | | | GAATTTGAAAT |
| | | | | ATTTTTATCAGAAAAATGGCATATTGCTGAAGTGTCAAAATATGAGAAAAGAGCA |
| | | | | TCTTCATTTTA |
| | | | | ATTAACTGCTTTATAAAAATGGCAACTTTAGGTATAGATAACCTAATTCAGGTTGC |
| | | | | AGTTTTAATAT |
| | | | | TCATTGGGGGCAGTGGAATTGCATGTGTAAATTATGTTAGATATATATCATAACA |
| | | | | ACCCTAGTGG |
| | | | | ATTCATGGAATTATAAACGTGATCCAAATTCTACTGTCTTCCAAGAAAGCTACATT |
| | | | | TATACATCCAA |
| | | | | ACAAATTCTTTTCCACAGCTTAAGAGTACCTCATCAGAATACAGTGGTTCAAGTTA |
| | | | | AAAAATAAAG |
| | | | | TCCATAATGCATATGGTCATCATTCTAGACCTGATAACCACTGAGAACCTGCTGA |
| | | | | GCAAGGACGG |
| | | | | CTTTTACCAACATTAGCACAAAGGTTTCCTCAGTTTAGACTCTAACATATGACCTG |
| | | | | AAACAACTCTA |
| | | | | GGTCTGAGTCCTAGAGATTACATAAATAGTGTGTAGCCCAGAAAAGTAAAATAG |
| | | | | GTGTCACGCTG |
| | | | | TTTTTTTTAATGTCAACACCAATAGGAATCCATTTAGATTAATGTATGTAATTTCTG |
| | | | | ATAAAAGTAA |
| | | | | GAGAAATGAGGAACTTTCGAACTGCAAATATGCCTCCAAACTAGCTTTTGATAAG |
| | | | | TGTGGTTTCT |
| | | | | GTTATTTGAATGGATTCCCATCTTTGTCTCTAGCCTGGAGAAGAGCAAAATGGAG |
| | | | | GGGGAGGGG |
| | | | | CCAGAAATAGGAGGAGGTTTTGAAGAATAAAGCCTCATATCCAGGAAAAAGTGA |
| | | | | TCAAGTGAAA |
| | | | | AAGCTGCATCAAGACCCATTTGGCAAGAGGTTGTCCGAAAATAATTCCTTGATCA |
| | | | | TATCTGAAGT |
| | | | | GGTTATTTACTCAAATCAATTTCTCCCCTGCTTCGCTAAGACTGTCATAAAATGGC |
| | | | | CTTGACATTTA |
| | | | | ATCACAGGTTACTTTCCAGAAAGGTTTCTAAAGTTACAGATTCAGCTTTTCAAATT |
| | | | | GTCAAATTCAC |
| | | | | TTTGAATAATAATAGAACCTGGGCTACATTCCTAACTCATATTAACAGCAC |
| 2115 | ETV1 | HSX2_00914001 | 228 | ATTTCGCCGCCAGCTTTCTGAACCCTGTAACTCCTTTCCTCCTTTGCCGACGATGCC |
| | | | | AAGGGAAGG |
| | | | | ACGTCCTATGTACCAACGCCAGATGTCTGAGCCAAACATCCCCTTCCCACCACAA |
| | | | | GGCTTTAAGCA |
| | | | | GGAGTACCACGACCCAGTGTATGAACACAACACCATGGTTGGCAGTGCGGCCAG |
| | | | | CCAAAGCTTT |
| | | | | CCCCCTCCTCTGATGATTAAACAGGAACCCAGAGATTTTGCATATGACTCAG |
| 2119 | ETV5 | 2119.005.1-D | 229 | TCAGTCAACTTCAAGAGGCTTGGTTAGC |
| 2119 | ETV5 | HSX2_00914314 | 230 | GGTGCGCGGAGCGGTTCACCGTCTTCGGAGCGGTTCGGCCCAGCCTTTCGCCCA |
| | | | | GGCGCCCAGG |
| | | | | CCCGCTGCGCGCGTGCGTGAGCGCGCCTGCGCCGCCGGGGCCGCTGCAAGGGG |
| | | | | AGGAGAGAG GCCGCCTCAG |
| 2119 | ETV5 | HSX2_00914317 | 231 | GAGGATCCCTTTTCCCCCAGAAATTACTCAATGCTGAAACCTCTCAAAGTGGTATT |
| | | | | AGAGACGCTG |
| | | | | AAAGCACCATGGACGGGTTTTATGATCAGCAAGTCCCTTTTATGGTCCCAGGG |
| 2119 | ETV5 | HSX2_00914321 | 232 | AAATCTCGATCTGAGGAATGCAGAGGGCGGCCTGTGATTGACAGAAAGAGGAA |
| | | | | GTTTTTGGACACAGATCTGGCTCACGATTCTGAAG |
| 2119 | ETV5 | HSX2_00914326 | 233 | AGCTATTTCAGGATCTCAGTCAACTTCAAGAGGCTTGGTTAGCTGAAG |
| 2119 | ETV5 | HSX2_00914331 | 234 | CACAAGTTCCTGATGATGAACAGTTTGTCCCAGATTTTCAGTCTGATAACC |
| 2119 | ETV5 | HSX2_00914336 | 235 | TGGTGCTTCATGCCCCACCTCCAACCAAGATCAAACGGGAGCTGCACAGCCCCTC |
| | | | | CTCTGAGCTG |
| | | | | TCGTCTTGTAGCCATGAGCAGGCTCTTGGTGCTAACTATGGAGAAAAGTGCCTCT |
| | | | | ACAACTATTG |
| 2119 | ETV5 | HSX2_00914372 | 236 | AAAAACGAAAAACAAAAAACAAAAACAAACCTTTTGGTAACTGTGCACTGTGTCC |
| | | | | TTTAGCCAGG |
| | | | | GCCGTGCCAACTTATGAAGACACTGCAGCTTGAGAGGGGCTTTGCTGAGGCTTC |
| | | | | CCCTTGGCCAT |
| | | | | GTGAAAGCCCGCCTTGTTGCCTGCTTTGTGCTTTCTGCACCAGACAACCTGATGG |
| | | | | AACATTTGCAC |
| | | | | CTGAGTTGTACATTTTTGAAGTGTGCAGGGCAGCCTGGACACAAGCTTAGATTCT |
| | | | | CTATGTATAGT |
| | | | | TCCCCGTGTTCACTAACATGCCCTCTCTGGAAAGCATATGTATATAACATGTGTCA |
| | | | | TGTCCTTTGG |
| | | | | AAACCTGGTCACCTGGTGAAAACCCTTGGGATTCTTCCCTGGGCATGACTGATGA |
| | | | | CAATTTCCATT |
| | | | | TCATCAGTTTGTTTTGTTTTCCTTTTTCTTTAAATCTTGGACTTTAAACCCTACCTGT |
| | | | | GTGATTCAGT |
| | | | | AGGGTTTGAGACTTACGTGTGATACTGACAGGTAAGCAACAGTGCTAGCATTCT |
| | | | | AGATTCCTGCC |
| | | | | TTTTTTTAAAAAGAAATTATTCTCATTGCTGTATTATATTGGAAAAGTTTTAAACA |
| | | | | ACCAAGCTAAA |
| | | | | GCTATGTGAAAGTTGAGCTCAAAGTAGAGGAAAAGTTACTGGTGGTACCTTGCT |
| | | | | GCCTGCTCTGC |
| | | | | TGGTAGAATTCTGTGCTCCCCGTGACACTTAGTACATTAAGAATGACTACACTGTT |
| | | | | CCTCGTATGT |
| | | | | GAAGGAGGCAGTGCTGACTCCGTGAGTGTGAGACACGTGCTTTGAACTGCTTTT |
| | | | | CTATTCATGGA |
| | | | | GCACTCCATAGTCTCAAACTGTCCCCCTTATGACCAACAGCACATTTGTGAAGAG |
| | | | | GTTCGCAGGG |
| | | | | ATAAGGGGTGCACTTTATAGCTATGGAAACATGAGATTCTCCTCTATTGGAAGCT |
| | | | | AATTAGCCCAC |
| | | | | AAAGGTGGTAAACCTGTAGATTGGGCCTTAATTAGCATTGTACTCTAATCAAAGG |
| | | | | ACTCTTTCTAA |
| | | | | ACCATATTTATAGCTTTCTTAACCTACACATAGTCTATACATAGATGCATATTTTAC |
| | | | | CCCCAGCTGG |
| | | | | CTAGAGATTTATTTGTTGTAAATGCTGTATAGATTTGGTTTTCCTTTCTTTACTTAC |
| | | | | CCTGGTTTGGA |
| | | | | TTTTTTTTTTTTTTCTTTTGAATGGATTTATGCTGTCTTAGCAATATGACAATAATCC |
| | | | | TCTGTAGCTT |
| | | | | GAGCTACCCCTCCCCTGCTGTAACTTACGTGACCTGTGCTGTCACTGGGCATAGG |
| | | | | ACAGCGGCAT |
| | | | | CACGGTTGCATTCCCATTGGACTCATGCACCTCCCGGATGGTTTTTGTTTTTTTCG |
| | | | | GGGGTTCTTTG |
| | | | | GGGTTTGTTTGTTTGCTTCTTTTCCAGAGTGTGGAAAGTCTACAGTGCAGAAAGG |
| | | | | CTTGAACCTGC |
| | | | | CAGCTGATTTGAAATACTTTCCCCTGCGCAGGGCCGTATGCATCCTGCCAAGCTG |
| | | | | CGTTATATTCT GTACTGTGTACAATAAAGAAGTTTGCTTTTCGTTTACCAA |
| 2167 | FABP4 | HSX2_02500430 | 237 | GAATGCGTCATGAAAGGCGTCACTTCCACGAGAGTTTATGAGAGAGCATAAGCC |
| | | | | AAGGGACGTT |
| | | | | GACCTGGACTGAAGTTCGCATTGAACTCTACAACATTCTGTGGGATATATTGTTC |
| | | | | AAAAAGATATT |
| | | | | GTTGTTTTCCATGATTTAGCAAGCAACTAATTTTCTCCCAAGCTGATTTTATTCAAT |
| | | | | ATGGTTACGT TGGTTAAATAAACTTTTTTTAGATT |
| 25987 | TSKU | HSX2_02546256 | 238 | ATGCTTCCCCGGGTGCCAATGCGAGGTGGAGACCTTCGGCCTTTTCGACAGCTTC |
| | | | | AGCCTGACTC |
| | | | | GGGTGGATTGTAGCGGCCTGGGCCCCCACATCATGCCGGTGCCCATCCCTCTGG |
| | | | | ACACAGCCCA |
| | | | | CTTGGACCTGTCCTCCAACCGGCTGGAGATGGTGAATGAGTCGGTGTTGGCGGG |
| | | | | GCCGGGCTAC |
| | | | | ACGACGTTGGCTGGCCTGGATCTCAGCCACAACCTGCTCACCAGCATCTCACCCA |
| | | | | CTGCCTTCTCC |
| | | | | CGCCTTCGCTACCTGGAGTCGCTTGACCTCAGCCACAATGGCCTGACAGCCCTGC |
| | | | | CAGCCGAGAG |
| | | | | CTTCACCAGCTCACCCCTGAGCGACGTGAACCTTAGCCACAACCAGCTCCGGGAG |
| | | | | GTCTCAGTGT |
| | | | | CTGCCTTCACGACGCACAGTCAGGGCCGGGCACTACACGTGGACCTCTCCCACAA |
| | | | | CCTCATTCAC |
| | | | | CGCCTCGTGCCCCACCCCACGAGGGCCGGCCTGCCTGCGCCCACCATTCAGAGCC |
| | | | | TGAACCTGG |
| | | | | CCTGGAACCGGCTCCATGCCGTGCCCAACCTCCGAGACTTGCCCCTGCGCTACCT |
| | | | | GAGCCTGGAT |
| | | | | GGGAACCCTCTAGCTGTCATTGGTCCGGGTGCCTTCGCGGGGCTGGGAGGCCTT |
| | | | | ACACACCTGT |
| | | | | CTCTGGCCAGCCTGCAGAGGCTCCCTGAGCTGGCGCCCAGTGGCTTCCGTGAGCT |
| | | | | ACCGGGCCT |
| | | | | GCAGGTCCTGGACCTGTCGGGCAACCCCAAGCTTAACTGGGCAGGAGCTGAGGT |
| | | | | GTTTTCAGGC |
| | | | | CTGAGCTCCCTGCAGGAGCTGGACCTTTCGGGCACCAACCTGGTGCCCCTGCCTG |
| | | | | AGGCGCTGCT |
| | | | | CCTCCACCTCCCGGCACTGCAGAGCGTCAGCGTGGGCCAGGATGTGCGGTGCCG |
| | | | | GCGCCTGGTG |
| | | | | CGGGAGGGCACCTACCCCCGGAGGCCTGGCTCCAGCCCCAAGGTGGCCCTGCAC |
| | | | | TGCGTAGACA |
| | | | | CCCGGGATTCTGCTGCCAGGGGCCCCACCATCTTGTGACAAATGGTGTGGCCCA |
| | | | | GGGCCACATA |
| | | | | ACAGACTGCTGTCCTGGGCTGCCTCAGGTCCCGAGTAACTTATGTTCAATGTGCC |
| | | | | AACACCAGTG |
| | | | | GGGAGCCCGCAGGCCTATGTGGCAGCGTCACCACAGGAGTTGTGGGCCTAGGA |
| | | | | GAGGCTTTGG |
| | | | | ACCTGGGAGCCACACCTAGGAGCAAAGTCTCACCCCTTTGTCTACGTTGCTTCCCC |
| | | | | AAACCATGA |
| | | | | GCAGAGGGACTTCGATGCCAAACCAGACTCGGGTCCCCTCCTGCTTCCCTTCCCC |
| | | | | ACTTATCCCCC |
| | | | | AAGTGCCTTCCCTCATGCCTGGGCCGGCCTGACCCGCAATGGGCAGAGGGTGGG |
| | | | | TGGGACCCCC |
| | | | | TGCTGCAGGGCAGAGTTCAGGTCCACTGGGCTGAGTGTCCCCTTGGGCCCATGG |
| | | | | CCCAGTCACT |
| | | | | CAGGGGCGAGTTTCTTTTCTAACATAGCCCTTTCTTTGCCATGAGGCCATGAGGC |
| | | | | CCGCTTCATCC |
| | | | | TTTTCTATTTCCCTAGAACCTTAATGGTAGAAGGAATTGCAAAGAATCAAGTCCAC |
| | | | | CCTTCTCATG |
| | | | | TGACAGATGGGGAAACTGAGGCCTTGAGAAGGAAAAAGGCTAATCTAAGTTCCT |
| | | | | GCGGGCAGTG |
| | | | | GCATGACTGGAGCACAGCCTCCTGCCTCCCAGCCCGGACCCAATGCACTTTCTTG |
| | | | | TCTCCTCTAAT |
| | | | | AAGCCCCACCCTCCCCGCCTGGGCTCCCCTTGCTGCCCTTGCCTGTTCCCCATTAG |
| | | | | CACAGGAGTA |
| | | | | GCAGCAGCAGGACAGGCAAGAGCCTCACAAGTGGGACTCTGGGCCTCTGACCA |
| | | | | GCTGTGCGGC |
| | | | | ATGGGCTAAGTCACTCTGCCCTTCGGAGCCTCTGGAAGCTTAGGGCACATTGGTT |
| | | | | CCAGCCTAGC |
| | | | | CAGTTTCTCACCCTGGGTTGGGGTCCCCCAGCATCCAGACTGGAAACCTACCCAT |
| | | | | TTTCCCCTGAG |
| | | | | CATCCTCTAGATGCTGCCCCAAGGAGTTGCTGCAGTTCTGGAGCCTCATCTGGCT |
| | | | | GGGATCTCCA |
| | | | | AGGGGCCTCCTGGATTCAGTCCCCACTGGCCCTGAGCACGACAGCCCTTCTTACC |
| | | | | CTCCCAGGAA |
| | | | | TGCCGTGAAAGGAGACAAGGTCTGCCCGACCCATGTCTATGCTCTACCCCCAGG |
| | | | | GTAGCATCTCA |
| | | | | GCTTCCGAACCCTGGGCTGTTTCCTTAGTCTTCATTTTATAAAAGTTGTTGCCTTTT |
| | | | | TAACGGAGTG |
| 27112 | FAM155B | HSX2_01700655 | 239 | GCTGTCTACAAGGCCTGGCTGTGCTCAGAATACTTCAGCGTGACCCAGCAGGAAT |
| | | | | GCCAGCGCT |
| | | | | GGGTGCCCTGCAAGCAATACTGCCTGGAGGTGCAGACCCGGTGCCCCTTTATACT |
| | | | | CCCCGACAAT |
| | | | | GAGGAAATGGTGTACGGAGGGCTCCCTGGCTTTATCTGTACAG |
| 27112 | FAM155B | HSX2_01700664 | 240 | GAGGCGAAGAAGAAGAAGTTCAAGGAGTCTGAGGCCCCCAAAACCCACCAGCA |
| | | | | GCAATTCCACC |
| | | | | ACTCCTATTTCCACCACTACCACCAACAGTACCATCACTACCACCCCCATCATGATC |
| | | | | CCCCAGGCC |
| | | | | GTGTCAGCAACAAGCCCGCCCTGCTGCCGGTCTCTGGGGGCTCCCGCCTCAGCCC |
| | | | | TAGCAGGAT |
| | | | | CCGGCTCTGCGTCCTTGTTCTCATGCTCCTCCATACCGTGGTGTCCTTCTCCAGCA |
| | | | | ACCAGGGTGG |
| | | | | TGGGGGATTGGGGCTGGAGACACTGCCTGCCCTAGAGGAGGGCCTGACACGGG |
| | | | | AAGAGTGACA |
| | | | | GTAGGGAGGGAGGACAGACCTCCACCACACTGACATCAGCTCCAGCTCCCCCAG |
| | | | | GTTGGGGGG |
| | | | | GAGGGGGCTCCTCCCATGGGAGGTGTAGGATAAGGTGGGGGCGGGGGAAATG |
| | | | | GGGGAATGAC |
| | | | | ACATCCCCCCCAACCTACCCCCACCCCAAAAGCAGCTCCAACAGAATGGCCAGAG |
| | | | | GGCCTCAGG |
| | | | | GAGCTGCAAAACTCATCCAGGAGAAAAAGGCAGGAGCAGCAAGTGACCCCTCCC |
| | | | | AAGCTCCCAT |
| | | | | CTATGGGGCTTAGCAAAAAAAGGGAGGAAGTGGGGGCTGGATATGCCCACCCC |
| | | | | TGCCAAAAGC |
| | | | | CCTGACCCCAGGGAAGGAGGCTGCTCACCCAGCCTTGGCCCTGCAGGGAATGTT |
| | | | | GGGGGGCAC |
| | | | | AGAGGGGAGAAGCTCTTTCCCCCACTCCACATTCCTTTTGTTGCCAAGATCCTTAA |
| | | | | TTCCCTCCTG |
| | | | | CCCATATCCCTACAGGCGACGGCAGACAGTGCAATGGCCCTCCTGCCACTTCAGC |
| | | | | ACACCTTGCC |
| | | | | CCACCTGGGACCATCACACGTGAAGCACCAGGCTGGGAGATGAGGTGCACACAG |
| | | | | TTGCAGCTAG |
| | | | | GTCGGGGCCCCAGTTAAGCTGTGCCCCACCACCCTAGGCAATGAGGGGCAGGAA |
| | | | | AGGGGTACA |
| | | | | GAATGAATGGTGAAAGAGAGTGGAGATACGGAAGGGGGAGAGGAGAGGAGAA |
| | | | | ATGTGGACAG |
| | | | | AGGGCTTGAAGACATGGCCGAATAGGCTACTGCCACCCGGCTTTGGGGAGATGG |
| | | | | GCGATAAGT |
| | | | | GTTGAGGTAGGCTCGAGAACTGCTCCCCAAATCAGTGAGAATTGCATTAGGAGC |
| | | | | CCTCTGGGGA |
| | | | | AAGAGCACAGGAACTGAGTTGCTGGGCCTCAAAACAAAAGGCTGCAGGCACATG |
| | | | | GTTAGACTGC |
| | | | | CGCTGTCTGAAGGACCATATCTTACAGAAGGTGCACATACTCCCAAGGGCCACTC |
| | | | | TTGCCCTGGA |
| | | | | GATCTTGGCCTTGGGGCCAAAGACATTTCCATTTCCGATCTCCATGGGGAGGAGG |
| | | | | GACTTAAGCC |
| | | | | CAAGTGGGTCAGGCCTGGCCCAGGTCCTTATACTCCCCTATCTTGTCCTAGCCCAG |
| | | | | GCCTCCATGA |
| | | | | AGGAGAACCTGGTGGCATTGCCCCAGAGCTCTCCAGAGATGAGCCTCCCCTATCC |
| | | | | CCACCCTGTC |
| | | | | CCCGTCCCCAGCCTACCAAAGAGTATTCACACCTTTCCATCCCTGACTCTATGGGT |
| | | | | TACTGTCCCA |
| | | | | ATTGCGAAACACCCATACTCCTCTTCACCCTGTAGCAGGGTCTTCTGCTCCAGTCA |
| | | | | CCCCGGCCTC |
| | | | | TTGCAAGAGAAAGTCCAGGGACTGGCAAGAAGACCCTTTGACTAAAGATAATGT |
| | | | | GCTGTCATATC |
| | | | | TTGGCTGAGAAACTGTTGAGTCAGGCTAGAGGACTGGACAAGAGAAGAGGCAT |
| | | | | TGAGAGCCCC |
| | | | | CTTGGGAGCCATTAGCCTTGCTCTGGTCTGCTGTGTGGGGTTGGGGAGTAGGGG |
| | | | | GGATCCCACA |
| | | | | TTGCCTGGGACATCTGAATGCTGATACGAATGGACAGGAAAGGGAGCCAGAGCT |
| | | | | GTATACCTGG |
| | | | | GACAGGCCAGGGAGCCTTTAGTGCCAGCAGGGCCTGTGTCCTGGGATGTGATGC |
| | | | | TAGTGAGTGA |
| | | | | ATGGGAGGGCGGGCCTGGAGCTGCGTGAGAGTGAAACCAAGAAGCGGATGGG |
| | | | | GAAGGGAGAG |
| | | | | CTGGCCTTGGTCAGCTTAGGGATGAATGGAGAAGGAATCATTCATGGGCCTCGG |
| | | | | CTAGGTCCCA |
| | | | | GACTGATTACATGAGACTGGGGAGTCCTTCCCTGCCAGTTTTCTCATTTATTACAG |
| | | | | CCTATCCCCC |
| | | | | AACCCTACCCCTGTGCCCTGCCTACACACTCCCGTTTGGCCCTCAGCACTTTGGGC |
| | | | | CTGGTCACAT |
| 165215 | FAM171B | HSX2_02270148 | 241 | GTGCTTGGGTAAATCATGGTCGGGGAATGGTCAAGGAACATAACAATCATTTAA |
| | | | | TCTGGACATAT |
| | | | | GATGCACCACATTTGGGGTACTGGATAGCAGCTCCACTTCCAGGAACTAGAG |
| 57184 | FAM219B | HSX2_01914824 | 242 | GTGAGCTTTGAATGGGCATAGACAGGGTTCCCAGCCCAATAGTCGGGATTGGGG |
| | | | | TATAGGTTCT |
| | | | | ACATGGGAAAGTATCCAGTCTCTTGCTTTACTCAGTGTCCTGTGACCACCAGAGG |
| | | | | CATGAGAGGG |
| | | | | ACAACACACTCTTTTCGCTCTACCACAAGGGGTGACTAGAAGATGAGCAAGTCTA |
| | | | | ATAAAAAGAT ACGGCCTTA |
| 284418 | FAM71E2 | HSX2_02337017 | 243 | TGGCAGGGCCATGGCGGAGGCCCGAGTCTCCCAGCCTGGGGCATCTCCACGCTC |
| | | | | TGTAACGCTG |
| | | | | AGCTCCAGGCACCCGTGAAGCCCCACGGGTCAAGGCTGGTGGGCCGGGGCTGG |
| | | | | GAGGCCTGCA |
| | | | | CGCCTGGGTTCTGGGTCCCTAAACCAGTACCCATCCACCACAGCCACCATGATCT |
| | | | | GGCTTCGAAA |
| | | | | CAGGAGGTGCCTTGAGCCGCTCCAGGGCACCCCGAAGTGGGTCCCTGTTCTGGG |
| | | | | GGAGCTGCAA AAGACCCTCCAGAAGG |
| 26224 | FBXL3 | HSX2_02546986 | 244 | GATGAAACGAGGAGGAAGAGATAGTGACCGTAATTCATCAGAAGAAGGAACTG |
| | | | | CAGAGAAATC |
| | | | | CAAGAAACTGAGGACTACAAATGAGCATTCTCAGACTTGTGATTGGGGTAATCTC |
| | | | | CTTCAGGACA |
| | | | | TTATTCTCCAAGTATTTAAATATTTGCCTCTTCTTGACCGGGCTCATGCTTCACAAG |
| | | | | TTTGCCGCAA |
| | | | | CTGGAACCAGGTATTTCACATGCCTGACTTGTGGAGATGTTTTGAATTTGAACTG |
| | | | | AATCAGCCAG |
| | | | | CTACATCTTATTTGAAAGCTACCCATCCAGAGCTGATCAAACAGATTATTAAAAGA |
| | | | | CATTCAAACC ATCTACAATATGTCAGCTTCAAG |
| 26234 | FBXL5 | HSX2_01687167 | 245 | AATGAATATGAACAGTTAAATTATGCAAAACAACTGAAAGAGAGATTGGAGGCT |
| | | | | TTTACAAGAGA TTTTCTTCCTCACATGAAAGAGGAAGAGGAG |
| 93611 | FBXO44 | HSX2_02575024 | 246 | AATTCCCCAATGACCAGGGTTCGCAGCCAGGCCA |
| 55294 | FBXW7 | HSX2_01846734 | 247 | CCGGACACACGGGGGCACAGAATCACTGAAGGGGAAAAATACAGAAAATATGG |
| | | | | GTTTCTACGGC ACATTAAAAATGATTTTTTACAAA |
| 342184 | FMN1 | HSX2_02583328 | 248 | ATGGAGAACGTTGATAATTCTCTGGATGGCAGTGATGTGTCAGAACCAGCAAA |
| | | | | ACCAGAAGCCG |
| | | | | GACTTGAAGTGGCTCAGTCGATCCTGAGTAAGTTCTCTATGAAATCCCTATTTG |
| | | | | GGTTTACGAGTA |
| | | | | AATTAGAATCTGTGAATCCAGAAGAGGAAGATGCTGTGCTGAAAGCATTCCAT |
| | | | | AGTTTAGACGTG |
| | | | | AATCCCACATCTCAGCAGGATGATTCCAGCAATGGCTTGGATCCACAGGAAGC |
| | | | | AGGGTCTCGGG |
| | | | | TTTCACCTGACCTCGGAAATGATGAGAAAATTGCAAGCGTGGAGACAGAATCA |
| | | | | GAGGGAAGTCA |
| | | | | GAGAAAAGAAGCAGGGACATCTCTCCTTGCTCAAGAGCTGCTTCCACTGTCTA |
| | | | | CTTTGAAAGGGA |
| | | | | CAAAAGACGATGTCATTTGTGTTCGTGGGACCCTGGTGCACACCACCAGTGAT |
| | | | | TCTGACTCTGAT |
| | | | | GATGGTGGCCAGGAGCCAGAGGAGGGAAGCAGCACCAATGGCCCAAAGTCC |
| | | | | CCTAGTGGTGTT |
| | | | | TTATCTGAGCCATCTCAGGAGTCCAAAGAAAACCCAGGAGGTTTCAGGGAAAA |
| | | | | TACTGTCACTGG |
| | | | | GGAAATGAATGGTGCAGAGCTCTGTGCAGAAGATCCTCAAAGGATTCCACCT |
| | | | | GAGATGAGCAGC |
| | | | | AAACTGGAAGCTGGCAACGGTGGTCTTCAGACAGAGCGTCGCCCCAGCCAGG |
| | | | | ACCAGGTGGGA |
| | | | | GAAGAAGGGTCCCAAGACCTACCTGCAGTAACAAACCAGAATTCGAGTGTTG |
| | | | | GTATCACTGAGA |
| | | | | GTGCCTCTTCTAAAAAAGAAGTCTCTGGAGAGAAGTCATTCCAACTTCCAGCTT |
| | | | | TTTTCAGTGGGC |
| | | | | TTCGTGTGCTGAAGAAGGGGGCTACCGCTGAGGGAGGAGAGACCATCACGG |
| | | | | AAATTAAACCAA |
| | | | | AGGATGGGGACTTAGCTTTGCTCAAATTGACGCAGCCTGTGCAGAAGTCCTTA |
| | | | | GTGCAGGCAGG |
| | | | | GCTGCAGACAGTGAAGAGTGAGAAGAAAGCAACTGATCCGAAGGCCACTCCC |
| | | | | ACTCTCCTGGAG |
| | | | | CAGCTCTCTCTGCTGCTCAACATTGACATGCCCAAAACTGAACCGAAGGGAGC |
| | | | | AGACCCTGAGTC |
| | | | | GCCCAGGAGAGAAGAGATGGGCTGCAATGCTGACCAAGAGAGCCAGAGCGG |
| | | | | CCCTGGAGTACC |
| | | | | ACAAACCCAGGGTGGTGAGGTCAAGCCAAAGTCGCCAGAGACAGCCCTGGAG |
| | | | | GCTTTTAAAGCC |
| | | | | CTATTCATCCGGCCCCCCAGAAAGGGGACCACAGCTGACACCTCTGAGCTGGA |
| | | | | AGCTCTCAAGC |
| | | | | GCAAGATGAGACATGAGAAGGAGTCGCTAAGAGCTGTGTTTGAACGGTCCAA |
| | | | | TTCTAAGCCAGC GGATGGCCCCTCTGATTCCAAAAGT |
| 257019 | FRMD3 | HSX2_02319439 | 249 | GCGAGCACCGCGCCGGCCCTGAGCCTCCCGCTCGCTCCCCACGGCCGCGGTGC |
| | | | | ATGTTCGCCTC |
| | | | | CTGCCACTGTGTGCCGAGAGGCAGGAGGACCATGAAAATGATCCACTTTCGG |
| | | | | AGCTCCAGCGTC |
| | | | | AAATCGCTCAGCCAGGAGATGAGATGCACCATCCGGCTGCTGGACGACTCGG |
| | | | | AGATCTCCTGCC ACATCCAG |
| 257019 | FRMD3 | HSX2_02319452 | 250 | AGGGAAACCAAAGGGCAGTTTCTCATTGACCACATCTGCAACTACTACAGCCT |
| | | | | GCTGGAGAAGG ACTACTTTGGCATTCGCTATGTGGACCCAGAGAAGCAAAGG |
| 257019 | FRMD3 | HSX2_02319480 | 251 | CTCATCCACCATACACCATGTGCTTTAGAGTGAAATTCTACCCACATGAACCCT |
| | | | | TGAAGATTAAAG AAGAGCTCACAAG |
| 257019 | FRMD3 | HSX2_02319488 | 252 | CTTCAAGCAAATGAAAACTCATCCACCATACACC |
| 257019 | FRMD3 | HSX2_02319496 | 253 | ATACCTTTTATACCTTCAGATTAAAAGGGACATTTTTCATGGCCGCCTGCTGTG |
| | | | | CTCCTTTTCTGAT GCTGCCTACCTGGGTGCCTGTATTGTTCAAG |
| 257019 | FRMD3 | HSX2_02319512 | 254 | CTGAGCTTGGTGATTACGATCCTGATGAGCATCCTGAGAATTACATCAGTGAG |
| | | | | TTTGAGATTTTCC |
| | | | | CCAAGCAGTCACAGAAGCTGGAAAGAAAAATAGTGGAAATTCATAAAAATGA |
| | | | | ACTCA |
| 257019 | FRMD3 | HSX2_02319534 | 255 | GGGGCAGAGCCCACCAGTTGCTGAATTTAACTTGCTCCTGAAAGCTCACACTTT |
| | | | | GGAAACCTACG GGGTGGATCCTCACCCATGCAAG |
| 257019 | FRMD3 | HSX2_02319546 | 256 | GATTCAACAGGCACAACAACATTTTTAGGATTCACAGCTGCAGGCTTTGTGGT |
| | | | | CTTTCAGGGAAA TAAGAGAATCCATTTGATAAAATG |
| 257019 | FRMD3 | HSX2_02319551 | 257 | ATCCTCACCCATGCAAGGATTCAACAGGCACAAC |
| 257019 | FRMD3 | HSX2_02319566 | 258 | AAAAAAGCCATGTTGGCATTCCATACTTCAACACCAGCTGCCTGCAAACATCTT |
| | | | | TGGAAGTGTGG AGTGGAAAACCAGGCCTTTTATAA |
| 257019 | FRMD3 | HSX2_02319579 | 259 | GTATGCAAAATCCAGTCAGATCAAGACTGTATCAAGCAGCAAGATATTTTTTAA |
| | | | | AGGAAGTAGAT TTCGATATAG |
| 257019 | FRMD3 | HSX2_02319588 | 260 | AACCAGGCCTTTTATAAGTATGCAAAATCCAGTC |
| 257019 | FRMD3 | HSX2_02319595 | 261 | TGGGAAAGTTGCCAAAGAGGTGGTGGAGGCCAGTTCCAAGATCCAGAGGGA |
| | | | | GCCTCCTGAGGT GCACAG |
| 257019 | FRMD3 | HSX2_02319604 | 262 | AGTAGATTTCGATATAGTGGGAAAGTTGCCAAAG |
| 257019 | FRMD3 | HSX2_02319610 | 263 | AGCCAACATTACTCAGAGCCGCAGTTCCCACTCCTTGAACAAACAGCTCATCAT |
| | | | | TAACATGGAACC |
| | | | | CCTGCAGCCCCTGCTTCCTTCCCCCAGCGAGCAAGAAGAAGAACTTCCTCTGG |
| | | | | GTGAGG |
| 257019 | FRMD3 | HSX2_02319619 | 264 | CCTCCTGAGGTGCACAGAGCCAACATTACTCAGA |
| 257019 | FRMD3 | HSX2_02319626 | 265 | GTGTTCCATTGCCTAAAGAGGAGAACATTTCTGCTCCCTTGATCTCCAGCTCCC |
| | | | | CAGTGAAGGCA |
| | | | | GCCCGGGAGTATGAAGATCCCCCTAGTGAAGAGGAAGATAAAATAAAAGAAG |
| | | | | AACCTTTAACCA |
| | | | | TCTCTGAACTAGTGTACAACCCAAGTGCCAGCCTGCTCCCCACCCCTGTGGATG |
| | | | | ACGATGAGATT |
| | | | | GACATGCTCTTTGACTGTCCTTCTAGGCTTGAGTTGGAAAGAGAAGACACAGA |
| | | | | TTCATTTGAGGA |
| | | | | TCTGGAAGCAGATGAAAACGCCTTTTTGATTGCTGAAGAAGAGGAGCTGAAG |
| | | | | GAGGCTCGCCGT |
| | | | | GCTTTGTCGTGGAGCTATGACATTCTGACTGGCCATATTCGGGTGAACCCACT |
| | | | | GGTCAAGAGTTT |
| | | | | TTCCAGGCTCCTTGTGGTGGGCCTGGGACTGCTGCTCTTTGTATTTCCCCTGCT |
| | | | | CCTCCTCCTTTTG GAGTCAG |
| 257019 | FRMD3 | HSX2_02319629 | 266 | GTATTGATCTCTCCTTCTTATGCGAAATCCGCCAGACACCAGAGTTTGAGCAGT |
| | | | | TTCACTATGAAT |
| | | | | ACTACTGTCCCCTCAAGGAGTGGGTGGCTGGGAAAGTCCACCTCATCCTCTAC |
| | | | | ATGCTGGGTTGC |
| | | | | TCATGAAGTTAATCTCTCACGTGACTAAGGGCTATATTCAATGCTAGTGATTTC |
| | | | | TTTTTTTCAGCAA |
| | | | | ATGCCTGGTTCTGAAGGGTCACGGGGCTGTCAACAGGTGTTCCTTACTCATAA |
| | | | | TTGATTATTCAAA |
| | | | | CCTTTAAGTTAGCTTTCCATAATTCACTGCACTTAAATAAGTTTAAATCAAATAC |
| | | | | AGTTATTTTAGTT |
| | | | | ACAGGTTAGGAAGATGGTCTTTAAATAACCAAAAATATGTTTATTTTTTATTAT |
| | | | | AGTGTAGACATA |
| | | | | CCCTTCATCTATTATATCATAATACATGTTACATTGGACTGAATTAGATTTTCCC |
| | | | | ATTTCTAATAGTT |
| | | | | GGCACCATTATAAGCTATAAGGTTCAGAATCAGAATTTTAGTAACAACTCAAG |
| | | | | AGAAAGTTGTTG |
| | | | | AATATAATCCTTAGTGAAAACAGTGTCCTCTAACCAATGCCTATACAACTAAAT |
| | | | | TTATGCTGGGTT |
| | | | | TTTGGTTCTGTTTTTTTAAAAATATTTTTATGTGTTCAAACTATTTTGGTAAATTT |
| | | | | TTAGCAA |
| 257019 | FRMD3 | HSX2_02319637 | 267 | CCTCCTTTTGGAGTCAGGTATTGATCTCTCCTTC |
| 257019 | FRMD3 | HSX2_02319641 | 268 | ACTTCCTCTGGGTGAGGGTGTTCCATTGCCTAAA |
| 257019 | FRMD3 | HSX2_02581974 | 269 | CTGAGCTTGGTGATTACGATCCTGATGAGCATCCTGAGAATTACATCAGTGAG |
| | | | | TTTGAGATTTTCC |
| | | | | CCAAGCAGTCACAGAAGCTGGAAAGAAAAATAGTGGAAATTCATAAAAATGA |
| | | | | ACTCAG |
| 122786 | FRMD6 | HSX2_02188584 | 270 | ACTATATGTCGGAAACCAAAGACCTCCACTGATCGACACAGCTTGAGCCTCGA |
| | | | | TGACATCAGACT |
| | | | | TTACCAGAAAGACTTCCTGCGCATTGCAGGTCTGTGTCAGGACACTGCTCAGA |
| | | | | GTTACACCTTTG |
| | | | | GATGTGGCCATGAACTGGATGAGGAAGGCCTCTATTGCAACAGTTGCTTGGCC |
| | | | | CAGCAGTGCAT |
| | | | | CAACATCCAAGATGCTTTTCCAGTCAAAAGAACCAGCAAATACTTTTCTCTGGA |
| | | | | TCTCACTCATGA |
| | | | | TGAAGTTCCAGAGTTTGTTGTGTAAAGTCCGTCTGTGTGCAGCTGTACAGGCA |
| | | | | GCTTACTGTTTGC |
| | | | | TAGAGGATGCGAAAGTCATAAGTTCTTTACATATTACTTGTGCCATATCTTCTTC |
| | | | | ACCCTAAACATA |
| | | | | GCTCTTTCTTTATAATATTTGTGATGATGGAAACAAAAGCCTTGGAACAATTGC |
| | | | | ACTTTAAGTATTA |
| | | | | CACAGAAGTAAAAGAACTACAGAAAATGTACAGCAAGACAAGTGCCCGGAAG |
| | | | | TTCACTGATCCT |
| | | | | TCAGAAGGAAATGCGCTTTACTGATTGCAAAGCCTTCAGAATATTGGAGTGTG |
| | | | | GTGTGTTTGCTC |
| | | | | ATCTGATGCTTTTTAGTTCAGTTACATGTAACATCACATTTTTTTATCACGTGAA |
| | | | | AGATGTTAGATT |
| | | | | TGTTTGCTTATAAATTTTTTACCACTCCCACATAAAATGCTCATAGTTTGGGAGA |
| | | | | GGAAAGAGGGA |
| | | | | AGATTCTCTCTTCTTTTAACAGAGAGATGATTGCTCTGTATACCCATTGCTTCCT |
| | | | | CCCTGAGGCTGT |
| | | | | CCCAAAGTGAACACTGATGGAGTGGTCAAAATCATAAGGTTGTAGCAAGCCAA |
| | | | | AGATACGTATG |
| | | | | TGACAGAAGCACATAAGCAATAAGCAGAAAACCAGAAGTGCATGCTGTGATG |
| | | | | CCTGTGACTCCTT |
| | | | | CATCCCGCTCAGTGCCATGTCCTCTTTTGTGATCTTCCAGAAAGCTCCAGGATTC |
| | | | | ATTTGAGTTCCA CATCCAAGTAACAGATGAATTATATTCATGTTGTAATGCA |
| 8321 | FZD1 | HSX2_01329270 | 271 | GACCCCGGCGCGCCCTAGCCACCCGGGTTCTCCCCGCCGCCCGCGCTTCATGA |
| | | | | ATCGCAAGTTTC |
| | | | | CGCGGCGGCGGCGGCTGCGGTACGCAGAACAGGAGCCGGGGGAGCGGGCC |
| | | | | GAAAGCGGCTTG |
| | | | | GGCTCGACGGAGGGCACCCGCGCAGAGGTCTCCCTGGCCGCAGGGGGAGCC |
| | | | | GCCGCCGGCCGT |
| | | | | GCCCCTGGCAGCCCCAGCGGAGCGGCGCCAAGAGAGGAGCCGAGAAAGTAT |
| | | | | GGCTGAGGAGG |
| | | | | AGGCGCCTAAGAAGTCCCGGGCCGCCGGCGGTGGCGCGAGCTGGGAACTTTG |
| | | | | TGCCGGGGCGC |
| | | | | TCTCGGCCCGGCTGGCGGAGGAGGGCAGCGGGGACGCCGGTGGCCGCCGCC |
| | | | | GCCCGCCAGTTG |
| | | | | ACCCCCGGCGATTGGCGCGCCAGCTGCTGCTGCTGCTTTGGCTGCTGGAGGCT |
| | | | | CCGCTGCTGCTG |
| | | | | GGGGTCCGGGCCCAGGCGGCGGGCCAGGGGCCAGGCCAGGGGCCCGGGCC |
| | | | | GGGGCAGCAAC |
| | | | | CGCCGCCGCCGCCTCAGCAGCAACAGAGCGGGCAGCAGTACAACGGCGAGCG |
| | | | | GGGCATCTCCG |
| | | | | TCCCGGACCACGGCTATTGCCAGCCCATCTCCATCCCGCTGTGCACGGACATCG |
| | | | | CGTACAACCAG |
| | | | | ACCATCATGCCCAACCTGCTGGGCCACACGAACCAGGAGGACGCGGGCCTGG |
| | | | | AGGTGCACCAGT |
| | | | | TCTACCCTCTAGTGAAAGTGCAGTGTTCCGCTGAGCTCAAGTTCTTCCTGTGCT |
| | | | | CCATGTACGCGC |
| | | | | CCGTGTGCACCGTGCTAGAGCAGGCGCTGCCGCCCTGCCGCTCCCTGTGCGAG |
| | | | | CGCGCGCGCCA |
| | | | | GGGCTGCGAGGCGCTCATGAACAAGTTCGGCTTCCAGTGGCCAGACACGCTC |
| | | | | AAGTGTGAGAAG |
| | | | | TTCCCGGTGCACGGCGCCGGCGAGCTGTGCGTGGGCCAGAACACGTCCGACA |
| | | | | AGGGCACCCCG |
| | | | | ACGCCCTCGCTGCTTCCAGAGTTCTGGACCAGCAACCCTCAGCACGGCGGCGG |
| | | | | AGGGCACCGTG |
| | | | | GCGGCTTCCCGGGGGGCGCCGGCGCGTCGGAGCGAGGCAAGTTCTCCTGCCC |
| | | | | GCGCGCCCTCA |
| | | | | AGGTGCCCTCCTACCTCAACTACCACTTCCTGGGGGAGAAGGACTGCGGCGCA |
| | | | | CCTTGTGAGCC |
| | | | | GACCAAGGTGTATGGGCTCATGTACTTCGGGCCCGAGGAGCTGCGCTTCTCGC |
| | | | | GCACCTGGATT |
| | | | | GGCATTTGGTCAGTGCTGTGCTGCGCCTCCACGCTCTTCACGGTGCTTACGTAC |
| | | | | CTGGTGGACAT |
| | | | | GCGGCGCTTCAGCTACCCGGAGCGGCCCATCATCTTCTTGTCCGGCTGTTACAC |
| | | | | GGCCGTGGCCG |
| | | | | TGGCCTACATCGCCGGCTTCCTCCTGGAAGACCGAGTGGTGTGTAATGACAAG |
| | | | | TTCGCCGAGGA |
| | | | | CGGGGCACGCACTGTGGCGCAGGGCACCAAGAAGGAGGGCTGCACCATCCTC |
| | | | | TTCATGATGCTC |
| | | | | TACTTCTTCAGCATGGCCAGCTCCATCTGGTGGGTGATCCTGTCGCTCACCTGG |
| | | | | TTCCTGGCGGCT |
| | | | | GGCATGAAGTGGGGCCACGAGGCCATCGAAGCCAACTCACAGTATTTTCACCT |
| | | | | GGCCGCCTGGG |
| | | | | CTGTGCCGGCCATCAAGACCATCACCATCCTGGCGCTGGGCCAGGTGGACGGC |
| | | | | GATGTGCTGAG |
| | | | | CGGAGTGTGCTTCGTGGGGCTTAACAACGTGGACGCGCTGCGTGGCTTCGTGC |
| | | | | TGGCGCCCCTC |
| | | | | TTCGTGTACCTGTTTATCGGCACGTCCTTTCTGCTGGCCGGCTTTGTGTCGCTCT |
| | | | | TCCGCATCCGCA |
| | | | | CCATCATGAAGCACGATGGCACCAAGACCGAGAAGCTGGAGAAGCTCATGGT |
| | | | | GCGCATTGGCGT |
| | | | | CTTCAGCGTGCTGTACACTGTGCCAGCCACCATCGTCATCGCCTGCTACTTCTA |
| | | | | CGAGCAGGCCTT |
| | | | | CCGGGACCAGTGGGAACGCAGCTGGGTGGCCCAGAGCTGCAAGAGCTACGCT |
| | | | | ATCCCCTGCCCT |
| | | | | CACCTCCAGGCGGGCGGAGGCGCCCCGCCGCACCCGCCCATGAGCCCGGACT |
| | | | | TCACGGTCTTCA |
| | | | | TGATTAAGTACCTTATGACGCTGATCGTGGGCATCACGTCGGGCTTCTGGATCT |
| | | | | GGTCCGGCAAG |
| | | | | ACCCTCAACTCCTGGAGGAAGTTCTACACGAGGCTCACCAACAGCAAACAAGG |
| | | | | GGAGACTACAG |
| 2560 | GABRB1 | HSX2_00936262 | 272 | GATTATACACTCACCATGTATTTCCAGCAGTCTTGGAAAGACAAAAGGCTTTCT |
| | | | | TATTCTGGAATC |
| | | | | CCACTGAACCTCACCCTAGACAATAGGGTAGCTGACCAACTCTGGGTACCAGA |
| | | | | CACCTACTTTCT |
| | | | | GAATGACAAGAAATCATTTGTGCATGGGGTCACAGTGAAAAATCGAATGATTC |
| | | | | GACTGCATCCTG ATGGAACAGTTCTCTATGGACTCCG |
| 2561 | GABRB2 | HSX2_00936319 | 273 | GTCCCCCCGTGGCTGTGGGGATGAACATTGACATTGCCAGCATCGATATGGTT |
| | | | | TCTGAAGTCAAT ATG |
| 2561 | GABRB2 | HSX2_00936324 | 274 | GATTATACCTTGACAATGTACTTTCAACAAGCCTGGAGAGATAAGAGGCTGTC |
| | | | | CTATAATGTAATA |
| | | | | CCTTTAAACTTGACTCTGGACAACAGAGTGGCAGACCAGCTCTGGGTGCCTGA |
| | | | | TACCTATTTCCTG |
| | | | | AACGATAAGAAGTCATTTGTGCACGGAGTGACTGTTAAGAACCGCATGATTCG |
| | | | | CCTGCATCCTGA TGGCACCGTCCTTTATGGACTCAG |
| 2561 | GABRB2 | HSX2_00936329 | 275 | AATCACAACCACAGCTGCCTGCATGATGGACCTAAGGAGGTACCCACTGGATG |
| | | | | AACAAAACTGC ACCTTGGAAATTGAGAGCT |
| 2561 | GABRB2 | HSX2_00936349 | 276 | ATGGACCCCCATGAGAACATCTTACTGAGCACTCTCGAGATAAAAAATGAAAT |
| | | | | GGCCACATCTGA |
| | | | | GGCTGTGATGGGACTTGGAGACCCCAGAAGCACAATGCTAGCCTATGATGCCT |
| | | | | CCAGCATCCAG |
| | | | | TATCGGAAAGCTGGGTTGCCCAGGCATAGTTTTGGCCGAAATGCTCTGGAACG |
| | | | | ACATGTGGCGC |
| | | | | AAAAGAAAAGTCGCCTGAGGAGACGCGCCTCCCAACTGAAAATCACCATCCCT |
| | | | | GACTTGACTGAT |
| | | | | GTGAATGCCATAGATCGGTGGTCCCGCATATTCTTCCCAGTGGTTTTTTCCTTCT |
| | | | | TCAACATCGTCT |
| | | | | ATTGGCTTTATTATGTGAACTAAAACATGGCCTCCCACTGGAAGCAAGGACTA |
| | | | | GATTCCTCCTCAA |
| | | | | ACCAGTTGTACAGCCTGATGTAGGACTTGGAAAACACATCAATCCAGGACAAA |
| | | | | AGTGACGCTAAA |
| | | | | ATACCTTAGTTGCTGGCCTATCCTGTGGTCCATTTCATACCATTTGGGTTGCTTC |
| | | | | TGCTAA |
| 2561 | GABRB2 | HSX2_00936350 | 277 | CTAGACAGCCTGAGTGCTCCATGTTGTCTGTTTAGTATGCAGCCTACTCAGAGG |
| | | | | GGAATAATTTG |
| | | | | GAAGATATTCTTAGAAGGCTCAACAGCATTGTCAGTCATTTGGTCATAACCAG |
| | | | | ATACTCCTCATCT |
| | | | | TCCTTTTTAAGGTCGGCATGCTGTTGAGATGGCATTGTCCTTATGAAACATTAT |
| | | | | CTGTAATGCCAT |
| | | | | GCAAACACATTACTTGAAGATGATCTATGATTTTCTTACTGAGATGATAATGGT |
| | | | | GTCATGGGAAG |
| | | | | ATTATTCCCACTGGTTTCTAAAAGATTCTCTTGTGATTTGAGCAGGCTAGGGGA |
| | | | | ATCAAGACAGA |
| | | | | GACTCAGGAAACCGATTTGGCCAGTCCCTTTGCTTGAGCACGGAGAATCAACT |
| | | | | ATGATGTCTTTC |
| | | | | ATTTTGGCATTTAGAGATGAGGAGACTTCTGGACAATATCATGCATGGTGGCC |
| | | | | AAGAAAATTTTT |
| | | | | GCTAAACGGCTATTTCCTAGGTGGTTAAAGTAAGAGTTAACAACACAGAGCAG |
| | | | | AGTAGAAAATCA |
| | | | | GAAACGACGGTTGTAACAAAAAGCAAGCAAGCAAGAAAGCATGACCCTTATC |
| | | | | AGCTCCACACAG |
| | | | | CATCTGGTCTATACATACTGTGTCTAGTGGCAGAGGGTGAGTAAGAGAGATTG |
| | | | | TTTTTCCAACAT |
| | | | | CACTAGCTTTTGACAGCATACCTAAAGAGGTTTTTATAGTTTAATCGAAGATAA |
| | | | | CCATGAAATACA |
| | | | | AAAATTAGAGAAATGAGAATTGTCTTAGATATTCAGGTTAGATTCAGAGTCAT |
| | | | | TCAAATATACAAA |
| | | | | TACAGAGCACAAACTAGCAAACAGATTGTTTTTCTCAGACGAAGTCAGCTGTCC |
| | | | | CTGCAATTTTTG |
| | | | | TCCCTATAATTTTATTTCTTAATTTAATAGAGTATTTCAAGAGAAGGATTTGTTC |
| | | | | ATCACTTTAAAG |
| | | | | GATTCATGGTCCTCTCCAGCTTTTTTTTTGCTTCCAAAATTCTGATTATATTCTTA |
| | | | | GGAGACTGGCA |
| | | | | AAGTAAGGCTTTGGAGAGATTGAGTGAACTATATTTTCTTATATGCAATGAAAT |
| | | | | TCACCATTGTAT |
| | | | | TTTTCAAAGCAAATAATACTTCAAGGAGTATGAACACACAGAGAGTTAATTTGT |
| | | | | GCATTCATTGGT |
| | | | | TTGGGGCTTGATTTTCTTAAGAGTGCTTTCATTGTATATGTTGCAATCTGCCTAA |
| | | | | TCAAGGAAAAT |
| | | | | ATGAAAACTAGTGGAAAATAATAACAAAGCCACATTAACATGATAAATGTTAT |
| | | | | GAGCCTCAGTTG |
| | | | | ATATTGCAGAAAAATATATAACTGTTAATCAGGCAGGCATGATGTATTTTTTTT |
| | | | | CTATTTTTAAGTT |
| | | | | ATGCCATTGTGCTTCTTTCTACACTAATTGGACATAAGCATTCATATGTTGGACA |
| | | | | AGAGGAGAATA |
| | | | | AGCTAATATGTCTTCCTTCTATCAATATAAGGCTCATATTTTGCTTTTCTTATAGA |
| | | | | AAATAGTGAAT |
| | | | | TGCTAAATTTTCCTTAAAGTTGAATTTGATTGAACTTTTTCTTGGAGGATGACTT |
| | | | | TGCTATTTCTATT |
| | | | | TTGAAAACAAAGAAAGCTCTTCTTTTTAGAGTAATATCCCTAATTGTGCATAGC |
| | | | | AGCCAGTATCCA |
| | | | | CCTGTGAATAATGTATGTTTTCTTGATTCCTTGTTTCCTTCATGATTTAGCAGCTT |
| | | | | GGAATATGATG |
| | | | | GAGTACTATTGCCCTTGCATTCCAGCTGTCCAAGAACAACAACAAACCTTTATC |
| | | | | CTTCAGCTTTCTG |
| | | | | AATTTGCCCCAAAATGGAATGGTATTTTGTTTTTCACCAGTGTAGGAAACCTTG |
| | | | | GCCATTATATTTT |
| | | | | GATTACATAATCTTGAGCACCTCTGAGTATCATCTTTGGGCAGAATCTGACCAA |
| | | | | GATTCTTCTCAC |
| | | | | AGGCTCAGAGTAGAGATTCCTCATGTTTCTCCAGGACACACTTTCTCCTAGGCA |
| | | | | ATCATTTTGGGG |
| | | | | CATCACACTAAGTAAAGTGCCTGCCCCTTGTCAACAGGGGGTAAAGTTTGACT |
| | | | | AAAAAACATCAG |
| | | | | GAAGGAACATATTGATAACTCAGGGGCACAACTCCAAAATGCCATTCTGAAAA |
| | | | | GGCTCTTCTAGA |
| | | | | GTCAACTCAGAGCCCAAGTGTAGAAGTGGGCTCTTATGTGCAATCACTGTACA |
| | | | | AGCCATAAAAAT |
| | | | | CTTTGATATTGCCATTTATGATTACAACAAAATTTTAGATGTCCAAGAAAGTCA |
| | | | | ACAGAACAATCC |
| | | | | AGATCAAAGTGTAGAGAAGCATCTTTGCAGCTTGAGCCTCAAAAGCATAAAAC |
| | | | | CAGGAATTCATT |
| 2561 | GABRB2 | HSX2_02488302 | 278 | GCCTGGATGTCAACAAGATGGACCCCCATGAGAA |
| 2563 | GABRD | HSX2_00936493 | 279 | GAGTACACCATGACGGTGTTCCTGCACCAGAGCTGGCGGGACAGCAGGCTCT |
| | | | | CCTACAACCACA |
| | | | | CCAACGAGACCCTGGGTCTGGACAGCCGCTTCGTGGACAAGCTGTGGCTGCCC |
| | | | | GACACCTTCATC |
| | | | | GTGAACGCCAAGTCGGCCTGGTTCCACGACGTGACGGTGGAGAACAAGCTCA |
| | | | | TCCGGCTGCAGC CCGACGGCGTGATCCTGTACAGCATCCG |
| 2569 | GABRR1 | HSX2_00936876 | 280 | TAATAATGGCCGTAAGCTTAAAATAGATCCAGGGAGGAGCTCATTAACGTGAA |
| | | | | CATAGAAAGCA |
| | | | | GTTCCGCACCTCTGGCCTTACTCCTCTTGGAAATTGCTTTGGTCCATTTTTACTT |
| | | | | CCTTTTATTCGAC |
| | | | | GCACCAGAAAATAAGACTTTTACCAACATTTTTACTGCATTTGACGATGAACTA |
| | | | | ATTTAGACCGGC |
| | | | | TAAAATAATTGTTCCACTGGGACACAGGAATTCAACCTCAGTTCAGAAAATCCC |
| | | | | TGACATCTGACG |
| | | | | TAGGAGGATTTATAGGTTTAGTGGAAATTGCTTTCTCCTGCTCTCCAGATTGCA |
| | | | | TCCTGTGGGTTG |
| | | | | ATTTTTTTTTTGCATGAGTAAACATCCTTCTAATAATGAACAGACCAATAATGTC |
| | | | | TTAAGAGAGAA |
| | | | | AAAGAACAATCTTTTCCTTTTTGCTGTTTCTGGAGAGAGCTGTTTGAATTTGG |
| 2581 | GALC | HSX2_00937669 | 281 | ATTACCCATTTTTTAGTGAAGCTCCAAACTTTGCTGATCAAACTGGTGTATTTGA |
| | | | | ATATTTTACAAA |
| | | | | TATTGAAGACCCTGGCGAGCATCACTTCACGCTACGCCAAGTTCTCAACCAGA |
| | | | | GACCCATTACAT |
| | | | | GGGCTGCCGATGCATCCAACACAATCAGTATTATAGGAGACTACAACTG |
| 51809 | GALNT7 | HSX2_01783826 | 282 | GGGGAGAGCGGTGGCGGCGGCTGCGCCGGGCTGTGAGTCTCTCGCCGCCGG |
| | | | | AGGAAGATGAG |
| | | | | GCTGAAGATTGGGTTCATCTTACGCAGTTTGCTGGTGGTGGGAAGCTTCCTGG |
| | | | | GGCTAGTGGTCC |
| | | | | TCTGGTCTTCCCTGACCCCGCGGCCGGACGACCCAAGCCCGCTGAGCAGGATG |
| | | | | AGG |
| 51809 | GALNT7 | HSX2_01783838 | 283 | AAACAAGCAATTCAAGCCAGCATTAAAGAGTTTGGATTTAACATGGTGGCAAG |
| | | | | TGACATGATCTC ACTGGACCGCAGCGTCAATGACTTACGCCAAGAAGA |
| 51809 | GALNT7 | HSX2_01783857 | 284 | GTTTTGATATACCTTGATGCCCACTGTGAGGTGGCAGTTAACTGGTATGCACCA |
| | | | | CTTGTAGCTCCC ATATCTAAGGACAG |
| 51809 | GALNT7 | HSX2_01783878 | 285 | AATTATGTTAGAGTTGTGGAGGTTTGGTGGGATGAATATAAAGACTACTTCTA |
| | | | | TGCTAGTCGTCCT |
| | | | | GAATCGCAGGCATTACCATATGGGGATATATCGGAGCTGAAAAAATTTCGAGA |
| | | | | AGATCACAACT |
| | | | | GCAAAAGTTTTAAGTGGTTCATGGAAGAAATAGCTTATGATATCACCTCACACT |
| | | | | ACCCTTTGCCAC CCAAAAATGTTGACTGGGGAGAA |
| 51809 | GALNT7 | HSX2_01783883 | 286 | ATCAGAGGCTTCGAAACTGCTTACTGCATTGATAGCATGGGAAAAACAAATGG |
| | | | | AGGCTTTGTTGA ACTAGGACCCTGCCACAGGATGGGAGGGAATCAG |
| 51809 | GALNT7 | HSX2_01783895 | 287 | AACCTGCACAGATTTACTCATATTCCTTCAGGAAAGTGTTTAGATCGCTCAGAG |
| | | | | GTCCTGCATCAA |
| | | | | GTATTCATCTCCAATTGTGACTCCAGTAAAACGACTCAAAAATGGGAAATGAAT |
| | | | | AACATCCATAGT GTTTAGAGAGAA |
| 51809 | GALNT7 | HSX2_02553388 | 288 | GAAGACAGAGATGTCAATGACCCCATGCCCAACCGAGGCGGCAATGGACTAG |
| | | | | CTCCTGGGGAG |
| | | | | GACAGATTCAAACCTGTGGTACCATGGCCTCATGTTGAAGGAGTAGAAGTGG |
| | | | | ACTTAGAGTCTAT |
| | | | | TAGAAGAATAAACAAGGCCAAAAATGAACAAGAGCACCATGCTGGAGGAGAT |
| | | | | TCCCAGAAAGAT |
| | | | | ATCATGCAGAGGCAGTATCTCACATTTAAGCCTCAGACATTCACCTACCATGAT |
| | | | | CCTGTGCTTCGC |
| | | | | CCAGGGATCCTCGGTAACTTTGAACCCAAAGAACCTGAGCCTCCTGGAGTGGT |
| | | | | TGGTGGCCCTGGAGAGAAAGCCAAGCCATTGGTTTTGGGACCAGAATTC |
| 168391 | GALNTL5 | HSX2_02580131 | 289 | ACTGTGTGGCCCCGCAACCCAGCACAACCAGGTATCTGCTTGGAACCCAGCCA |
| | | | | CCATAAAGCCTG |
| | | | | CTAGCTAAAAAAAATTTTACATCTCTCAGTTCATTCGGCACAGACCCCTGCCTC |
| | | | | ATTCAGCTGTGA |
| | | | | CTCTGCTTGGAAAATTCATCAGTTACAAAGCAGCCAATGCAATTATCTCAAGG |
| 2595 | GANC | HSX2_00939002 | 290 | GGTTCTTCAGTGGGTGAGTTATATCTTGATGATGGCCATTCATTCCAATACCTC |
| | | | | CACCAGAAGCAA |
| | | | | TTTTTGCACAGGAAGTTTTCATTCTGTTCCAGTGTTCTGATCAATAG |
| 2635 | GBP3 | HSX2_00941807 | 291 | GAACAGGCCCGAGTACTAAAGGAGAGATGCCAAGGTGAAAGTACCCAACTTC |
| | | | | AAAATGAGATAC |
| | | | | AAAAGCTACAGAAGACCCTGAAAAAAAAAACCAAGAGATATATGTCGCATAA |
| | | | | GCTAAAGATCTA |
| | | | | AACAACAGAGCTTTTCTGTCATCCTAACCCAAGGCATAACTGAAACAATTTTAG |
| | | | | AATTTGGAACAA |
| | | | | GTGTCACTATATTTGATAATAATTAGATCTTGCATCATAACACTAAAAGTTTACA |
| | | | | AGAACATGCAG |
| | | | | TTCAATGATCAAAATCATGTTTTTTCCTTAAAAAGATTGTAAATTGTGCAACAA |
| | | | | AGATGCATTTACC |
| | | | | TCTGTACCAACAGAGGAGGGATCATGAGTTGCCACCACTCAGAAGTTTATTCTT |
| | | | | CCAGACGACCA |
| | | | | GTGGATACTGAGGAAAGTCTTAGGTAAAAATCTTGGGACATATTTGGGCACTG |
| | | | | GTTTGGCCAAGT |
| | | | | GTACAATGGGTCCCAATATCAGAAACAACCATCCTAGCTTCCTAGGGAAGACA |
| | | | | GTGTACAGTTCT |
| | | | | CCATTATATCAAGGCTACAAGGTCTATGAGCAATAATGTGATTTCTGGACATTG |
| | | | | CCCATGG |
| 2635 | GBP3 | HSX2_02501985 | 292 | GTACCAGTCAAAAAGATAAAAATTTTAATCTGCCCCGACTCTGTATCCGGAAGT |
| | | | | TCTTCCCAAAGA |
| | | | | AAAAATGTTTTGTCTTCGATCTGCCCATTCACCGCAGGAAGCTTGCCCAGCTTG |
| | | | | AGAAACTACAAG |
| | | | | ATGAAGAGCTGGACCCTGAATTTGTGCAACAAGTAGCAGACTTCTGTTCCTAC |
| | | | | ATCTTTAGCAATT |
| | | | | CCAAAACTAAAACTCTTTCAGGAGGCATCAAGGTCAATGGGCCTC |
| 6843 | VAMP1 | HSX2_01248411 | 293 | GGCGGGACTGAAGAGCTGGAAGAAAGCAGTCAGTACCCTCCCAACGGCCCCC |
| | | | | CTCGAAGGTCTC |
| | | | | CACTCTCCTCTGGGCTCCTCCTTGCCTAATGCAGGGGGTCACCGCTGGAGAAG |
| | | | | AACCACCACTGT |
| | | | | CCTCGATGTGTCCCAAGCCTGGAGCGAATCCGTCCTCTTGGCTCTCCCAGCCCT |
| | | | | ATCACAGGAATC |
| | | | | ATTCCTGGGTTTCTGTCCCTCTGAGGCTCACCAGGTGTAGTTGGCCTTGTTCTTT |
| | | | | GGGGGTATTAG |
| | | | | CCCTTCTACTTTCTTTCTCACCCACCCTGTACCCTCTTCTGTGTGTCTCTGCTATC |
| | | | | CCCCTTTCCTCCC |
| | | | | ACCACCCATGTGCATGAGCAAATGTGCAACAAAACCCTGGGACTTTGCAGTCA |
| | | | | AATGAAGCTGAG |
| | | | | CTTCCCACATCCCGTTCTTCGGTTTGCCATGAGACGATGTGGGGTTTCCACTGT |
| | | | | GTGTCTGTCATC |
| | | | | ACCTCTTTGTCTTTTTTTCCTAACCCTGATCTCATACTTGTATAGAGTAATAACAA |
| | | | | CAGTTGTACTTC |
| | | | | CACCAAAGGCATGTGGCATATTTACCAATGTCATGTATTCTGAACAAAGGCAA |
| | | | | AAAATACAAATT CCTACCATTAA |
| 6843 | VAMP1 | HSX2_02520727 | 294 | ATGATGATCATGCTGGGAGCCATCTGTGCCATCATCGTGGTAGTTATTGTAA |
| 10810 | WASF3 | HSX2_01528591 | 295 | GACAGAGTGCCCAGCGGGTCACATGCATCGGACG |
| 9203 | ZMYM3 | HSX2_01391230 | 296 | CCAAACCCATGCGTATCAAAGAGGATATTCTCGCCTGCTCAGCTGCTGAGCTCA |
| | | | | ACTACGGTCTG |
| | | | | GCCCAGTTTGTGAGAGAAATCACTCGGCCCAATGGTGAACGATATGAACCTGA |
| | | | | CAGTATCTACTA TTTGTGTCTTGGCATCCAGCAG |
| 140688 | NOL4L | HSX2_02221814 | 297 | GGCCCACGGACCTCAGCATGAAAGGCGGGGCCTCTACCACCTCCACCACCCCC |
| | | | | ACGCCC |
| 84527 | ZNF559 | HSX2_02102522 | 298 | CTGAGAGCCACAGTCAGGTCTGTCCTCAGGGGTC |
| 23015 | GOLGA8A | HSX2_01586004 | 299 | GAATAATCGGTGGAGATGGATTGTGAAATGGAGGTGCATAAATATCCAGTTT |
| | | | | GCCAACCGAGCT AAGAAATGCTGGATAAAAGTAAAAG |
| 440270 | GOLGA8B | HSX2_02404961 | 300 | AGCTTGGAGCAAAGAAGATGATGTCTCACTCTGT |
| 6196 | RPS6KA2 | HSX2_01198733 | 301 | CCTTTCAGACGGAAGGAAAGCTCTACCTGATCCTGGACTTCCTGCGGGGAGGG |
| | | | | GACCTCTTCACC CGGCTCTCCAAAGAG |
| 6196 | RPS6KA2 | HSX2_01198797 | 302 | GTTGTTCATCGAGACCTGAAGCCGAGTAACATCCTGTACAGGGATGAGTCGGG |
| | | | | GAGCCCAGAAT |
| | | | | CCATCCGAGTCTGCGACTTCGGCTTTGCCAAGCAGCTGCGCGCGGGGAACGG |
| | | | | GCTGCTCATGAC ACCCTGCTACACGGCCAATTTCGTGGCCCCGGAG |
| 2859 | GPR35 | HSX2_00954161 | 303 | GGTGGCCCTTTGGGCTCCCTGTCCAGGATTTGCGCTCTGGAGGGTAGGGCTTG |
| | | | | CTGGGCTGGGG |
| | | | | ACTGGAGGGGAACGTGGAGCTCCTTCTGCCTCCTTTCCTGCCCCATGACAGCA |
| | | | | GGCAGATCCCAG |
| | | | | GAGAGAAGAGCTCAGGAGATGGGAAGAGGATCTGTCCAGGGGTTAGACCTC |
| | | | | AAGGGTGACTTG |
| | | | | GAGTTCTTTACGGCACCCATGCTTTCTTTGAGGAGTTTTGTGTTTGTGGGTGTG |
| | | | | GGGTCGGGGCT |
| | | | | CACCTCCTCCCACATCCCTGCCCAGAGGTGGGCAGAGTGGGGGCAGTGCCTTG |
| | | | | CTCCCCCTGCTC ACTCTCTGCTGACCTCCGGCTCCCTGTGCTGCCCCAG |
| 51704 | GPRC5B | HSX2_01779589 | 304 | AACACGCCCAACTACTTCGACACGTCGCAGCCCAGGATGCGGGAGACGGCCTT |
| | | | | CGAGGAGGACG |
| | | | | TGCAGCTGCCGCGGGCCTATATGGAGAACAAGGCCTTCTCCATGGATGAACAC |
| | | | | AATGCAG |
| 2869 | GRK5 | HSX2_00954458 | 305 | CCCCGCGCTGTGTACTGTAAGGACGTGCTGGACATCGAGCAGTTCTCCACTGT |
| | | | | GAAGGGCGTCA |
| | | | | ATCTGGACCACACAGACGACGACTTCTACTCCAAGTTCTCCACGGGCTCTGTGT |
| | | | | CCATCCCATGGC AAAACGAG |
| 2869 | GRK5 | HSX2_00954468 | 306 | TCTACTGTCTCAGTTTACATTTTGTATATTTGTATTTAAATGAAAGTGAGACTTT |
| | | | | GAGGGTGTATAT |
| | | | | TTTCTGTGCAGCCACTGTTAAGCCATGTGTTCCAAGGCATTTTAGCGGGGAGG |
| | | | | GGGTTATCAAAA |
| | | | | AAAAAAAAATGTGACTCAAGACTTCCAGAGCCTCAAATGAGAAAATGTCTTTA |
| | | | | TTAAATGTAGAA AGTGATCCATA |
| 54103 | GSAP | HSX2_01789812 | 307 | GAAGTTTGTGTGTATGTTACAGCCCGAAGTGTGCCTCTTGGGGACAAATCACA |
| | | | | TATTCAGTGTTTT ACATTCATAAAG |
| 54103 | GSAP | HSX2_01789986 | 308 | ATCTGGATAATACAGAGAAAAATGAAAAACTGAAATTCAGTATCATTGTGCGG |
| | | | | CTTCCTCCG |
| 373156 | GSTK1 | HSX2_02371777 | 309 | GCCTTTGGGCTGCCCATCACCGTGGCCCATGTGGATGGCCAAACCCACATGTT |
| | | | | ATTTGGCTCTGA CCGGATGGAGCTGCTGGCGCACCTGCTGG |
| 84705 | GTPBP3 | HSX2_02572437 | 310 | AGGAGGGGGTCCTGGAGGCTCTGGCCCACGTGGA |
| 727936 | GXYLT2 | HSX2_02430028 | 311 | AGTGTCTCTATGTATTCCCCTGCCAGTGGAACTACCGTCCCGATCACTGCATGT |
| | | | | ACGGAAGCAACT |
| | | | | GCAGAGAGGCTGAGCATGAAGGTGTGTCTGTTCTGCATGGAAACCGAGGCGT |
| | | | | CTACCATGACGA |
| | | | | TAAGCAACCAACGTTCAGAGCACTCTATGAAGCAATACGGGAT |
| 727936 | GXYLT2 | HSX2_02430046 | 312 | TTTCCCTTTCAAGACAATCTCTTTCAATCCATGTATTACCCCCTTCAGCTGAAGTT |
| | | | | TTTGGAGACTG |
| | | | | TGCACACTTTATGTGGACGAATCCCGCAAGTTTTTCTGAAGCAAATTGAGAAA |
| | | | | ACAATGAAAAGG |
| | | | | GCTTATGAGAAACACGTCATCATCCATGTTGGCCCCAACCAGATGCACTGAAT |
| | | | | ATTTTGTCTTGTT |
| | | | | GCAAGTCAATTAGGTGTCTTGTGACCAAGGAAATACTAATCTCTAAGCTGCCT |
| | | | | GGGTCTTTT |
| 727936 | GXYLT2 | HSX2_02430047 | 313 | TGTGTGAATATTTAATGGTGCTCCATGACTGTTGAGTTTTAAAAACCTCGTTAA |
| | | | | ATTTTGCCAAATC |
| | | | | AGTTGCCCCCAAAAGGGAATATGCTTTTCCTTATTTTTTTTTCTAAAATGCTATT |
| | | | | TATCTCTAAGGA A |
| 3014 | H2AFX | HSX2_00965768 | 314 | GTTTGAGCCGTCGTGCTTCACCGGTCTACCTCGCTAGCATGTCGGGCCGCGGC |
| | | | | AAGACTGGCGG |
| | | | | CAAGGCCCGCGCCAAGGCCAAGTCGCGCTCGTCGCGCGCCGGCCTCCAGTTCC |
| | | | | CAGTGGGCCGT |
| | | | | GTACACCGGCTGCTGCGGAAGGGCCACTACGCCGAGCGCGTTGGCGCCGGCG |
| | | | | CGCCAGTGTAC |
| | | | | CTGGCGGCAGTGCTGGAGTACCTCACCGCTGAGATCCTGGAGCTGGCGGGCA |
| | | | | ATGCGGCCCGC |
| | | | | GACAACAAGAAGACGCGAATCATCCCCCGCCACCTGCAGCTGGCCATCCGCAA |
| | | | | CGACGAGGAGC |
| | | | | TCAACAAGCTGCTGGGCGGCGTGACGATCGCCCAGGGAGGCGTCCTGCCCAA |
| | | | | CATCCAGGCCGT |
| | | | | GCTGCTGCCCAAGAAGACCAGCGCCACCGTGGGGCCGAAGGCGCCCTCGGGC |
| | | | | GGCAAGAAGGC |
| | | | | CACCCAGGCCTCCCAGGAGTACTAAGAGGGCCCGCGCCGCGGCCGGCCGCCA |
| | | | | GGCCTCCCCATG |
| | | | | CCACCACAAAGGCCCTTTTAAGGGCCACCACCGCCCTCATGGAAAGAGCTGAG |
| | | | | CCGCTTCAGACT |
| | | | | GCGGGGCAAGCGGGCCGCGGCTCCCTTCCCCTCCCCTCCCCTCGCCCGCCTTCG |
| | | | | CCGCCCGGCCT |
| | | | | CGAGTCCCCGCCCGCCCCCGCTCCCGTCCCGCACCGCCTGCCGCGTCGGCCTCG |
| | | | | GGCCCTGCCCT |
| | | | | GTCCGCCGTCCGCCCTCCGGTAGGGTTCGGGCCTTCCGGATGCGGCTTGGGCG |
| | | | | CTCTTCGGGGA |
| | | | | CCTCCGTGGCGCGGAAGACCCGAGCCTGCCGGGGGGAGGCCGGCGGCGCCG |
| | | | | CACCTGCCCGCC |
| | | | | TCGGCGTTCGTGACTCAGCCGCCCCATCCCGAGTCGCTAAGGGGCTGCGGGG |
| | | | | AGGCCGCAGCAC |
| | | | | CTTCTGGAAGACTTGGCCTTCCGCTCTGACGCAGGGCCGAGGTGGGCAGTCCA |
| | | | | GGCCGAGAGGC |
| | | | | CGGCGGCCCTGAAGGTGAGTGAGGCCCTCGGCAGCTGCAGCCGGGGTGTCTG |
| | | | | GTACCCCCCCG |
| | | | | GCGTGGTGCTTAGCCCAGGACTTTCAGACGCGGCCGCTGGCCGGGAGGCTTT |
| | | | | GGTGGGAGAGA |
| | | | | CGCGATCGCCGATTTCGGTCTGGCGCCCCTTCTGCGGCCGGGACCCAGGCCTT |
| | | | | TCACATCAGCTC |
| | | | | TCCCTCCATCTTCATTCATAGGTCTGCGCTGGGGCCGGGACGAAGCACTTGGT |
| | | | | AACAGGCACATC |
| | | | | TTCCTCCCGAGTGACTGCCTCCTAGGAGGACATTTAGGGGAGGGCAGAGGCCT |
| | | | | GCAGTTTGGCT |
| | | | | TCACGGCTGGCTATGTGGACAGCAAGAGTCGTTTTCGCGGAAGCCGACTGGC |
| | | | | AGCCAGGCCTGT |
| | | | | CGGGCCCCCCGACGCCGCCCCATTTCCCTTCCAGCAAACTCAACTCGGCAATCC |
| | | | | AAGCACCTAGA |
| | | | | TACCAGCACAAGTCGGTTAATCCCTGTCTGGACTGAGCCTCCGTTGGCTTCTGA |
| | | | | ACTGGAATTCTG |
| | | | | CAGCTAACCCTTCCACGACTAGAACCTTAGGCATTGGGGAGTTTTAGATGGAC |
| | | | | TAATTTTATTAAA GGATTGTTTTTTTTTTAA |
| 57493 | HEG1 | HSX2_01924755 | 315 | TTGGAGAACATCAGCTTGCCAGCAGCTCTGAGGTGCAAAATGGAAGTCCCATG |
| | | | | TCTCAGACTGA |
| | | | | GACTGTGTCTAGGTCAGTCGCACCCATGAGAGGTGGAGAGATCACTGCACACT |
| | | | | GGCTCTTGACC |
| | | | | AACAGCACAACATCTGCAGATGTGACAGGAAGCTCTGCTTCATATCCTGAAGG |
| | | | | TGTGAATGCTTC |
| | | | | AGTGTTGACCCAGTTCTCAGACTCTACTGTACAGTCTGGAGGAAGTCACACAG |
| | | | | CATTGGGAGATA |
| | | | | GGAGTTATTCAGAGTCTTCATCTACATCTTCCTCGGAAAGCTTGAATTCATCAG |
| | | | | CACCACGTGGAG AACGTTCGA |
| 57493 | HEG1 | HSX2_01924813 | 316 | ACAGTTCACGCCTCTAGGGAGTCCAACGCGGTGG |
| 57493 | HEG1 | HSX2_01924845 | 317 | AAAGAATAAAAATGACATAAGCAAACTCATCTTCAAAAGTGGAGATTTCCAAA |
| | | | | TGTCCCCGTATG |
| | | | | CTGAATACCCCAAAAATCCTCGCTCACAAGAATGGGGCCGAGAAGCTATTGAA |
| | | | | ATGCATGAGAAT |
| | | | | GGAAGTACCAAAAACCTCCTCCAGATGACGGATGTGTACTACTCG |
| 57493 | HEG1 | HSX2_01924856 | 318 | CGGATGTGTACTACTCGCCTACAAGTGTAAGGAA |
| 9931 | HELZ | HSX2_01455269 | 319 | ATTTTTAGAACAAGGAATCTGTAGGTATGGTGCCCAGTGTACTTCAGCACATTC |
| | | | | CCAGGAAGAAC |
| | | | | TAGCAGAATGGCAGAAAAGATATGCTTCACGGCTGATAAAATTGAAACAGCA |
| | | | | AAATGAGAATAA |
| | | | | ACAGCTCTCAGGCAGTTACATGGAAACCTTGATAGAAAAGTGGATGAATTCAT |
| | | | | TGTCTCCTGAGA AAGTG |
| 9931 | HELZ | HSX2_01455291 | 320 | GTTCAACCTTAAAGTGCAATTGCAGATTCTGGCAAGCTTCATGCTCACTGGTGT |
| | | | | TTCTGGAGGTGC |
| | | | | AAAGTATGCTCAGAATGGACAACTTTTTGGTCGCTTTAAGCTTACTGAAACACT |
| | | | | TTCTGAAGATAC |
| | | | | TTTGGCTGGACGACTGGTGATGACCAAAGTCAATGCTGTTTATTTATTACCAGT |
| | | | | CCCTAAACAGAA |
| | | | | GTTAGTACAGACCCAGGGAACCAAAGAGAAGGTTTATGAAGCTACTATTGAA |
| | | | | GAAAAAACAAAG |
| | | | | GAATATATATTTTTAAGGCTATCTAGGGAATGCTGTGAAGAACTTAATCTTCGG |
| | | | | CCTGACTGTGAC ACACAG |
| 9931 | HELZ | HSX2_01455342 | 321 | TTTGGGAACGGTTTATTGCCCTGTGTCATGAAAACAGTAGCCTACATGGAATC |
| | | | | ACTTTTGAACAGA |
| | | | | TCAAAGCCCAGTTAGAGGCTTTAGAACTAAAGAAGACATATGTGTTGAATCCG |
| | | | | CTGGCACCTGAA |
| | | | | TTTATCCCCCGGGCTCTAAGACTGCAGCATTCAGGAAGTACCAACAAACAGCA |
| | | | | GCAATCACCACC CAAG |
| 9931 | HELZ | HSX2_01455369 | 322 | CTCAGCTTTCTCCTGCATATCAGGCGGGACCCAACAATGCTTTTTTTAATAGTG |
| | | | | CAGTTGCTCATC |
| | | | | GGCCACAGTCTCCTCCTGCAGAAGCTGTAATTCCGGAGCAGCAGCCCC |
| 9931 | HELZ | HSX2_02533027 | 323 | GAAATTTTGGGAACGGTTTATTGCCCTGTGTCATGAAAACAGTAGCCTACATG |
| | | | | GAATCACTTTTGA |
| | | | | ACAGATCAAAGCCCAGTTAGAGGCTTTAGAACTAAAGAAGACATATGTGTTGA |
| | | | | ATCCGCTGGCAC |
| | | | | CTGAATTTATCCCCCGGGCTCTAAGACTGCAGCATTCAGGAAGTACCAACAAA |
| | | | | CAGCAGCAATCA CCACCCAAG |
| 23493 | HEY2 | HSX2_01639712 | 324 | ACCTCACGAGTGGAAATGTGGTATTCTCTTTTTTTTCTCTCCCTTTTTTGTTTGGT |
| | | | | TCAAGGCAGCT |
| | | | | CGGTAACTGACATCAGCAACTTTTGAAAACTTCACACTTGTTACCATTTAGAAG |
| | | | | TTTCCTGGAAAA |
| | | | | TATATGGACCGTACCATCCAGCAGTGCATCAGTATGTCTGAATTGGGGAAGTA |
| | | | | AAATGCCCTGAC |
| | | | | TGAATTCTCTTGAGACTAGATGGGACATACATATATAGAGAGAGAGTGAGAG |
| | | | | AGTCGTGTTTCGT |
| | | | | AAGTGCCTGAGCTTAGGAAGTTTTCTTCTGGATATATAACATTGCACAAGGGA |
| | | | | AGACGAGTGTGG |
| | | | | AGGATAGGTTAAGAAAGGAAAGGGACAGAAGTCTTGCAATAGGCTGCAGACA |
| | | | | TTTTAATACCAT |
| | | | | GCCAGAGAAGAGTATTCTGCTGAAACCAACAGGTTTTACTGGTCAAAATGACT |
| | | | | GCTGAAAATAAT |
| | | | | TTTCAAGTTGAAAGATCTAGTTTTATCTTAGTTTGCCTTCTTTGTACAGACATGC |
| | | | | CAAGAGGTGAC |
| | | | | ATTTAGCAGTGCATTGGTATAAGCAATTATTTCATCAGTTCTCAGATTAACAAG |
| | | | | CATTTCTGCTCTG |
| | | | | CCTGCAGGCCCCCAGGCACTTTTTTTTTTGGATGGCTCAAAATATGGTGCTGCT |
| | | | | TTATATAAACCTT |
| | | | | ACATTTATATAGTGCACCTATGAGCAGTTGCCTACCATGTGTCCACCAGAGGCT |
| | | | | ATTTAATTCATG |
| | | | | CCAACTTGAAAACTCTCCAGTTTGTAGGAGTTTGGTTTAATTTATTCAGTTTCAT |
| | | | | TAGGACTATTTT |
| | | | | TATATATTTATCCTCTTCATTTTCTCCTAATGATGCAACATCTATTCTTGTCACCC |
| | | | | TTTGGGAGAAGT |
| | | | | TACATTTCTGGAGGTGATGAAGCAAGGAGGGAGCACTAGGAAGAGAAAAGCT |
| | | | | ACAATTTTTAAA |
| | | | | GCTCTTTGTCAAGTTAGTGATTGCATTTGATCCCAAAACAAGATGAATGTATGC |
| | | | | AATGGGATGTAC |
| | | | | ATAAGTTATTTTTGCCCATGCCTAAACTAGTGCTATGTAATGGGGTTGTGGTTT |
| | | | | TGTTTTTTTCGAT |
| | | | | TTCGTTTAATGACAAAATAATCTCTTAATATGCTGAAATCAAGCACGTGAGAGT |
| | | | | TTTTGTTTAAAA |
| | | | | GATAAGAGACACAGCATGTATTATGCACTTCATTTCTCTACTGTGTGGAGAAA |
| | | | | GCAATAAACATTA |
| | | | | TGAGAATGTTAAACGTTATGCAAAATTATACTTTTAAATATTTGTTTTGAAATTA |
| | | | | CTGTACCTAGTC |
| | | | | TTTTTTGCATTACTTTGTAACCTTTTTCTATGCAAGAGTCTTTACATACCACTAAT |
| | | | | TAAATGAAGTCC TTTTTGACTA |
| 3098 | HK1 | HSX2_00971987 | 325 | GACTATGATGCCAACATCGTAGCTGTGGTGAATGACACAGTGGGCACCATGAT |
| | | | | GACCTGTGGCT ATGACGACCAGCACTGTGAAGTCGGCCTGATCATCG |
| 9455 | HOMER2 | HSX2_01407795 | 326 | AGAACAGCCCATCTTCACCACCCGAGCGCATGTCTTCCAGATTGACCCCAACAC |
| | | | | CAAGAAGAACT |
| | | | | GGATGCCTGCGAGCAAGCAGGCGGTCACCGTTTCCTACTTCTATGATGTCACA |
| | | | | AGGAACAGCTAT CGGATCATCAGTGTGGACGGAGCCAAG |
| 3249 | HPN | HSX2_00981105 | 327 | AACCGGGTCCTGTCCCGATGGCGAGTGTTTGCCGGTGCCGTGGCCCAGGCCTC |
| | | | | TCCCCACGGTCT |
| | | | | GCAGCTGGGGGTGCAGGCTGTGGTCTACCACGGGGGCTATCTTCCCTTTCGGG |
| | | | | ACCCCAACAGC |
| | | | | GAGGAGAACAGCAACGATATTGCCCTGGTCCACCTCTCCAGTCCCCTGCCCCTC |
| | | | | ACAG |
| 3249 | HPN | HSX2_00981117 | 328 | GAGGCTCGAGTCCCCATAATCAGCAATGATGTCTGCAATGGCGCTGACTTCTA |
| | | | | TGGAAACCAGAT |
| | | | | CAAGCCCAAGATGTTCTGTGCTGGCTACCCCGAGGGTGGCATTGATGCCTGCC |
| | | | | AG |
| 3249 | HPN | HSX2_02504427 | 329 | GCCAACAGGCCGGGGTACTCCAGGAGGCTCGAGTCCCCATAATCAGCAATGA |
| | | | | TGTCTGCAATGG |
| | | | | CGCTGACTTCTATGGAAACCAGATCAAGCCCAAGATGTTCTGTGCTGGCTACCC |
| | | | | CGAGGGTGGCA TTGATGCCTGCCAG |
| 90161 | HS6ST2 | HSX2_02132501 | 330 | TTCAATTTTCTTTAGTTTTAATTGAAAATGTTTGCTTTTAAAACTGATAGGTATT |
| | | | | GTTGGAAAGCAG |
| | | | | GATGAAGCCTGAGCCAGTGGAAAAGCTTGTTACAGAAAAAACATTTTGTGTTA |
| | | | | TTGCTGTGGTGT |
| | | | | GCATGATTTGCAAAGATTAAGTGCATTTTCTCTGTCTATACTGATTATTGTATAT |
| | | | | AGAGGATGTTAT |
| | | | | AAATATACATATACATTTTTGCCATTATGTAAATCCCATGATTTCAACTGTAAAC |
| | | | | ATCTGTCCATTG |
| | | | | GTGTAGCTTTACAAACCATTCACTGATTTTGTGTAATTTAACAATAGATATGAA |
| | | | | ATAAAGTTTAAAT TACA |
| 163702 | IFNLR1 | HSX2_02268031 | 331 | ATTGAACCACCTTCTTTCCTGGGGCAAGAGCACCAGGCTCCAGGGCACTCGGA |
| | | | | GGCTGGTGGGG |
| | | | | TGGACTCAGGGAGGCCCAGGGCTCCTCTGGTCCCAAGCGAAGGCTCCTCTGCT |
| | | | | TGGGATTCTTCA |
| | | | | GACAGAAGCTGGGCCAGCACTGTGGACTCCTCCTGGGACAGGGCTGGGTCCT |
| | | | | CTGGCTATTTGG |
| | | | | CTGAGAAGGGGCCAGGCCAAGGGCCGGGTGGGGATGGGCACCAAGAATCTC |
| | | | | TCCCACCACCTG |
| | | | | AATTCTCCAAGGACTCGGGTTTCCTGGAAGAGCTCCCAGAAGATAACCTCTCCT |
| | | | | CCTGGGCCACC |
| | | | | TGGGGCACCTTACCACCGGAGCCGAATCTGGTCCCTGGGGGACCCCCAGTTTC |
| | | | | TCTTCAGACACT |
| | | | | GACCTTCTGCTGGGAAAGCAGCCCTGAGGAGGAAGAGGAGGCGAGGGAATC |
| | | | | AGAAATTGAGGA |
| | | | | CAGCGATGCGGGCAGCTGGGGGGCTGAGAGCACCCAGAGGACCGAGGACAG |
| | | | | GGGCCGGACAT |
| | | | | TGGGGCATTACATGGCCAGGTGAGCTGTCCCCCGACATCCCACCGAATCTGAT |
| | | | | GCTGCTGCTGCC |
| | | | | TTTGCAAGGACTACTGGGCTTCCCAAGAAACTCAAGAGCCTCCGTACCTCCCCT |
| | | | | GGGCGGCGGA |
| | | | | GGGGCATTGCACTTCCGGGAAGTCCACCTAGCGGCTGTTTGCCTGTCGGGCTG |
| | | | | AGCAACAAGAT |
| | | | | GCCCCTCCCTCCTGTGACCCGCCCTCTTTAGGCTGAGCTATAAGAGGGGTGGA |
| | | | | CACAGGGTGGG |
| | | | | CTGAGGTCAGAGGTTGGTGGGGTGTCATCACCCCCATTGTCCCTAGGGTGACA |
| | | | | GGCCAGGGGGA |
| | | | | AAAATTATCCCCGGACAACATGAAACAGGTGAGGTCAGGTCACTGCGGACATC |
| | | | | AAGGGCGGACA |
| | | | | CCACCAAGGGGCCCTCTGGAACTTGAGACCACTGGAGGCACACCTGCTATACC |
| | | | | TCATGCCTTTCC |
| | | | | CAGCAGCCACTGAACTCCCCCATCCCAGGGCTCAGCCTCCTGATTCATGGGTCC |
| | | | | CCTAGTTAGGC |
| | | | | CCAGATAAAAATCCAGTTGGCTGAGGGTTTTGGATGGGAAGGGAAGGGTGGC |
| | | | | TGTCCTCAAATC |
| | | | | CTGGTCTTTGGAGTCATGGCACTGTACGGTTTTAGTGTCAGACAGACCGGGGT |
| | | | | TCAAATCCCAGC |
| | | | | TCTGCTGTTCACTGGTTGTATGATCTTGGGGAAGACATCTTCCTTCTCTGCCTCG |
| | | | | GCTTCCTCATCT |
| | | | | GCAGCTACGCCTGGGTGTGGTGAGGGTTCTAGGGGATCTCAGATGTGTGTAG |
| | | | | CACGGAGCCTGC |
| | | | | TGTGTCCTGGGTGCTCTCTACGTGGTGGCCGGTAGAATTCTCCATCTATCCAGG |
| | | | | CTCCAGGAGAC |
| | | | | CCCTGGGCATCTCCCACCTGTGGCCCCTAAACCCAGAGTGACTGAGAGCACTT |
| | | | | AACATTCAGCTT |
| | | | | GTCTCATCCCCAGTCTACCTCCTTCCTTCTACCCTCACTGCCTCCCAGTCAGGAG |
| | | | | AGTGAGCTCTCA |
| | | | | GAAGCCAGAGCCCCACCCAAGGGGACCCTGGTCTCTCCGCCTTCACCTAGCAA |
| | | | | TGGGAACCCTG |
| | | | | CTTCCCAGGGGAGGAACCAACTGCTCCACCTTCTAGGGACCCAGTTTGTTGGA |
| | | | | GTAGGACAGTAA |
| | | | | CATGGCAGGAATCGGACTTCTGGGCCTGTAATCCCAGTTTGGATGGCACGTTA |
| | | | | GACTCTTGGTTG |
| | | | | ACCGTTGTGGTCCTTAGAAGTCCCATTCTCCCTTCCAGTTATGAGAAACCAATG |
| | | | | CCTTCTAGATTC |
| | | | | AGGTGACTATCCTTACCTGGGGGTGCTGATGCATCCTCAGTTAACCTACACCCA |
| | | | | CCTGAATATAG |
| | | | | ATGAGCGTAGCTGAGTTTTCACCCGTAGGACCGAAGTGTTTTGTGGTGGAGTA |
| | | | | TCTGAACAACCT |
| | | | | TGGCTCTGTGGCCATTCAACCTGCCAGGACTAACATTTCTGGATTTGTGAAGAA |
| | | | | GGGATCTTCAA |
| | | | | AGCCATTGAACCCACAGAGCTGTGTTGCTTTAAAGCCACCACAAGGGTACAGC |
| | | | | ATTAAATGGCAG |
| | | | | AACTGGAAAAGCTTCTTAGGGCATCTCATCCAGGGATTCTCAAACCATGTCCCC |
| | | | | CAGAGGCCTTG |
| | | | | GGCTGCAGTTGCAGGGGGCGCCATGGGGCTATAGGAGCCTCCCACTTTCACCA |
| | | | | GAGCAGCCTCA |
| | | | | CTGTGCCCTGATTCACACACTGTGGCTTTCCACGTGAGGTTTTGTTTAGAGGGA |
| | | | | TCCACTACTCAA |
| 3476 | IGBP1 | HSX2_00991063 | 332 | GCATCAACTTCTAACTCATCTCGCCAGGAGAGGCCTCCAGTGAAACCCTTCATT |
| | | | | CTCACTCGGAAC ATGGCTCAA |
| 3489 | IGFBP6 | HSX2_00992250 | 333 | GGCCCATGCCGTAGACATCTGGACTCAGTGCTGCAGCAACTCCAGACTGAGGT |
| | | | | CTACCGAGGGG |
| | | | | CTCAAACACTCTACGTGCCCAATTGTGACCATCGAGGCTTCTA |
| 3489 | IGFBP6 | HSX2_02505340 | 334 | GGCCCATGCCGTAGACATCTGGACTCAGTGCTGCAGCAACTCCAGACTGAGGT |
| | | | | CTACCGAGGGG |
| | | | | CTCAAACACTCTACGTGCCCAATTGTGACCATCGAGGCTTCTACCGGAAGCGG |
| | | | | CAG |
| 285313 | IGSF10 | HSX2_02487762 | 335 | TGATGATACTGATTTATTTAATGGATCTCAAAACAAACTTTTAACTTAAGGCAC |
| | | | | TTTTATTTTGCCA |
| | | | | ACAAATAACAATAAACAAACATTGAAACGGTTCACTATAAAATAACAAATGGC |
| | | | | TAATGTACCTGA |
| | | | | ATTTTTCAGTAAAAAAATGAACTTCTAATACCAGTTGCCTAGTGTCCACCTCCTA |
| | | | | TCAATGTTACAA |
| | | | | GCATGGCACTCAGAACAGAGACAATGGAAAATATTAAATCTGCAATCTTTATG |
| | | | | ATGTAAATTTACC |
| | | | | ATCCTGATGTATAAATATTTTGTGGTTTATAAATTTTTTTGCTAAAACCTACAGA |
| | | | | AAATAAGCACTG |
| | | | | AACTGTCTTATGATTCACCATTTGTTCACACTGAAAATTTTCATTGATGGGGAAT |
| | | | | ACAAAAATGTTT |
| | | | | TAATAACTGCATTTAAAAATAAATGCAGGCTCACTTTTCCACATGTAAAAAGCA |
| | | | | ATGTAATACCTG |
| | | | | CACATCATAAAATGTGAAAAGGGGGTTATTTTTGTCCAAATACACCCATTAAAA |
| | | | | AGGTTTTAAAAA TACAA |
| 22807 | IKZF2 | HSX2_01567897 | 336 | CCCGACATTGTCACCTCCTCTTTGAGGGGTTAGAAGAAGCTGAGATCTCCCGA |
| | | | | CAGAGCTGGAAA TG |
| 22807 | IKZF2 | HSX2_01567903 | 337 | CCTCCGTTTATCTTCAGCCCGACATTGTCACCTC |
| 22807 | IKZF2 | HSX2_01567916 | 338 | CATTGCACTTTGACTATGGAAACAGAGGCTATTGATGGCTATATAACGT |
| 22807 | IKZF2 | HSX2_01567953 | 339 | GTGAACGCCCCTTCCACTGTAACCAGTGTGGAGCTTCTTTTACTCAGAAGGGCA |
| | | | | ACCTTCTGAGA |
| | | | | CACATAAAGTTACACTCTGGAGAGAAGCCGTTCAAATGTCCTTTCTGTAGCTAC |
| | | | | GCCTGTAGAAG AAGGGACGCCCTCACAGGACACCTCAGGACCCATTCTG |
| 22807 | IKZF2 | HSX2_01567978 | 340 | TACCTCCTATGGAAGATTGTAAGGAACAAGAGCCTATTATGGACAACAATATT |
| | | | | TCTCTGGTGCCTT |
| | | | | TTGAGAGACCTGCTGTCATAGAGAAGCTCACGGGGAATATGGGAAAACGTAA |
| | | | | AAGCTCCACTCC ACAAAAGTTTGTGG |
| 22807 | IKZF2 | HSX2_01568048 | 341 | GTAAACTGAAATGTGACGTCTGTGGCATGGTTTGCATTGGGCCCAATGTGCTT |
| | | | | ATGGTACATAAA AGGAGTCACACTG |
| 22807 | IKZF2 | HSX2_01568069 | 342 | TAAAAGGAGTCACACTGGTGAACGCCCCTTCCAC |
| 386653 | IL31 | HSX2_02379521 | 343 | GTGGAGGAAGAGAAGGGCGTGCTCGTGTCCCAGAATTACACGCTGCCGTGTC |
| | | | | TCAGCCCTGACG |
| | | | | CCCAGCCGCCAAACAACATCCACAGCCCAGCCATCCGGGCATATCTCAAGACA |
| | | | | ATCAGACAGCTA |
| | | | | GACAACAAATCTGTTATTGATGAGATCATAGAGCACCTCGACAAACTCATATT |
| | | | | TCAAGATGCACCA |
| | | | | GAAACAAACATTTCTGTGCCAACAGACACCCATGAATGTAAACGCTTCATCCT |
| | | | | GACTATTTCTCAA |
| | | | | CAGTTTTCAGAGTGCATGGACCTCGCACTAAAATCATTGACCTCTGGAGCCCA |
| | | | | ACAGGCCACCAC TTAAGGCCATCTCTTCCTTTCGGAT |
| 26034 | IPCEF1 | HSX2_01676158 | 344 | GTTTTCTCACGATGAGTCGGAGGAGGATATCGTGTAAAGATCTGGGCCATGCT |
| | | | | GACTGCCAAGG |
| | | | | GTGGCTGTATAAGAAAAAGGAAAAGGGAAGTTTCCTAAGCAACAAATGGAAA |
| | | | | AAGTTCTGGGTG |
| | | | | ATACTGAAGGGGTCGTCACTGTACTGGTATAGCAATCAAATG |
| 26034 | IPCEF1 | HSX2_01676239 | 345 | AATGTTACAGTGAAAGTGAACAGGAAGATCCAGAAATAGCTGCGGAGACACC |
| | | | | ACCCCCTCCTCA CGCTTCCCAGACTCAGTCTTTG |
| 26034 | IPCEF1 | HSX2_01676280 | 346 | AAGAGACAAAAGTGTCTGAAGATGATGAAATGGAGAAGCTGTACAAATCATT |
| | | | | AGAGCAAGCTAG |
| | | | | TCTATCTCCTCTTGGGGACCGACGACCTTCGACTAAAAAGGAGTTGAGAAAAT |
| | | | | CCTTTGTTAAGC |
| | | | | GGTGTAAAAATCCATCTATAAACGAGAAACTCCACAAAATCCGAACATTGAAT |
| | | | | AGCACATTAAAG |
| 26034 | IPCEF1 | HSX2_01676301 | 347 | TGTAAAGAACATGATCTGGCCATGATTAACCAGTTGCTGGATGACCCGAAGCT |
| | | | | GACAGCCAGGA |
| | | | | AATACAGAGAGTGGAAAGTCATGAACACCCTGCTGATCCAGGACATCTATCA |
| | | | | GCAGCAGCGGGC |
| | | | | TTCGCCTGCCCCTGATGACACTGATGACACCCCCCAGGAACTCAAGAAATCAC |
| | | | | CTTCTTCTCCCTC |
| | | | | TGTTGAAAATTCCATTTGAGACAAAGTCAGGGTTTTCTCCTCTT |
| 26034 | IPCEF1 | HSX2_01676305 | 348 | AAACCCAGTCCTTCAAGCATCAGCTTCCCATCTAAAGATGCACGTTAGATGAA |
| | | | | GATAATCACCGA |
| | | | | ATACACCAGGGCTCCTCATTTCAGTCGTAACGCATCCATTGAGATTGGAATGA |
| | | | | TTGAGTTCAGTTG |
| | | | | AAAGGCGTTTTTTGTGTTCCTACTGGGAGCTCACGGTGATGTGGGGCTCTAGG |
| | | | | GGGGATGTGAG |
| | | | | AAATGTCTCTTCTGTTCATTGCTCTCCAGAGAATGCTATGGACAAGAATCAAAT |
| | | | | TTTACCAGGAGT |
| | | | | AATTGCCCATGGAAACATTCTGTTTTTCAAAAGAAGAATGTACATATTCCACTT |
| | | | | GACTGGGAAGCT |
| | | | | GTGTGGCCCAAATCACTGTTTGAGTGGAGGAATGTTTTCTAGAGCAGTGACTG |
| | | | | GAAGGACAAAC |
| | | | | GTGGTGTTGCAGAACAAAAATTCCTTGCCTTTGGTATGGAATGGTGGTGCCTT |
| | | | | TTCCATGTGATG |
| | | | | AGAAATGCTTCATAGACAGTGGCATGTGGGAGAGCTGAAGGAGGAGGCGTG |
| | | | | TTCCATATTCCAC |
| | | | | TCATTCAAAGGACAGCTTGGTGTTTTCACACTTTATGCAGGTTAGGGGATCTG |
| | | | | ATTAATGTTTCCT |
| | | | | TTCCTTACAACAGCTAGAGAAAACATCTCATCACAATTGGAGACCTCTACTTCC |
| | | | | TTTGAGCTTTCCT |
| | | | | ACTCAATGGTAGGAGATAGGAATATGTTCCGCAAAGAATGTCTACTGCTCTTG |
| | | | | TCCTTGGCTGTG |
| | | | | ATCCCTATACATAGTGTGAATTTATATCCAGGTTTCCCAGGATCAATATTGACA |
| | | | | AGACGACTTTGG |
| | | | | CATCTGAATTGTTTTAATATCTTTACTCAAAAAAACAAAAAAACAAAAACCCAT |
| | | | | GGAAGCAACAAA |
| | | | | AGAGTTAAACATGAGGGATTTATTTTTGTATGTTTATATGTCACTTGTGTGCTA |
| | | | | TGTGAATAGCTAT |
| | | | | GTTTGTTATTTAAATATATTTATAGAAGTTCAAATTACTAGTGTTTAAAAGATG |
| | | | | ATATACTATTATA |
| | | | | TATTGTGCTTAGTATATATTTGATGAGTCTATACTTTTTGCTTGAGAAAAATAG |
| | | | | AAATACTATTGAT |
| | | | | TCACTGGTGTTATGATGTTGCACTGTGTCTGAGGAATGCTCAGGAAATCTTGT |
| | | | | GGGAAAAAAATG |
| | | | | GGGCATGCATTCATAATTTTTCATTTTCTTCCCCACCCCAACCCCCATGAGCTCT |
| | | | | TAAATTTAGATT |
| | | | | TCTATCTCTTGTAAGTCACTCTTTTAACCAGCAAAGGACTTTCATCTTAGATTAT |
| | | | | CCTTTCTATTAAA |
| | | | | TAAACTTTAAGCTTTGTGCCATTTATGCCTGCAAACCTTAGACCAAAGGCAACA |
| | | | | CAACTAATGTCA |
| | | | | TCTTTTCATTAAAGACTAATAGAAATTTAGAAATCATTGTGTTAAAGAGAGCTA |
| | | | | TAGAGAAACAAT |
| | | | | ATGAAAGTCTTCCCTTACTAATCACCATGAATATTTCTCACCATAGGAATAACC |
| | | | | ACAACCCACCAA |
| | | | | GTACCCATGTTGGCCCTGCCTACCCGTAACAAATTGTTCACACAACCTATTTTG |
| | | | | TTTTATAGCTTCA |
| | | | | AAGTGTGGCTTGACATTCAAATGGAAGGAACCACCTGAATTATATCTGCAAA |
| | | | | CAAAATGGAAATA |
| | | | | GTTGTTGAATAGTTCCCCAAGCTTTACTAAGAAGCCTTAATACTCTGTAAACA |
| | | | | AACAAACAAACAA |
| | | | | AAAACAACCTTAGCATTAAGAAATAAAAACAAAAAACAAGAAGCAAGTTGTA |
| | | | | CTGCTAGAAACTT |
| | | | | CTCTAAGATAAGAGGCCAGGCCAGTCAGTTTGGCCCAGCAAACATGTGGGTA |
| | | | | CTTCTCCACCTAG |
| | | | | GGAAGCCACTGAAGATGAGTTTGACCTGTGGTGACCTGACAGAGAGAACATT |
| | | | | CAGATCCTCAGA |
| | | | | TTGCACCCTGATGCCCTGGTTTAGGAAAGCAGTGCTTTAACCTTTTGCCCCTGG |
| | | | | AAATTACTGATT |
| | | | | CTTTCTGTGATTAGCAGATACCAGAAGGGAAATTATTTAGGATTGGCAATGTA |
| | | | | AAGAGTATTGAA |
| | | | | GGAACAACAAATAACAATAGCAGAAATAACAGTGTGACATAAAGTGCCCTTC |
| | | | | CACCTATTTTTCTT |
| | | | | TGGTTTGCAACACCAGCCTGTAGACTTCCAGTTTTTCTTAAATTGAAGAGCTCC |
| | | | | AGACACTTTTGA |
| | | | | GCAGGGTTTGTGAATAGCTTTGAATGCTTTCCTTTGGCTTTTCGTGAAGACATA |
| | | | | TTTCCCCAGCCTT |
| | | | | TCAACTGTAAACTCCTTTAGTACCATTGAAACTTAAGGCAAAAAAGATCCCTG |
| | | | | GGATTACTTAGCC |
| 3679 | ITGA7 | HSX2_01011260 | 349 | GAGTACTCAGCTGTGAAGTCCCTGGAAGTGATTGTCCGGGCCAACATCACAGT |
| | | | | GAAGTCCTCCAT AAAGAACTTGATGCTCCGAGATGCCTCCACAGTG |
| 3682 | ITGAE | HSX2_01011786 | 350 | TGTGGCTTTTTTAAAAGAAAATATCAACAACTGAACTTGGAGAGCATCAGGAA |
| | | | | GGCCCAGCTGAA |
| | | | | ATCAGAGAATCTGCTCGAAGAAGAGAATTAGGACCTGCTATCCACTGGGAGA |
| | | | | GGCTATCAGCCA GTCCTGGGACTT |
| 3685 | ITGAV | HSX2_01012164 | 351 | AGCCTCAGACGCTGCGTGGAGCGGCGGAGCCGGAGGGAAGCAAAGGACCGT |
| | | | | CTGCGCTGCTGT |
| | | | | CCCCGCCCCGCGCGCTCTGCGCCCCTCGTCCCTGGCGGTCGCTCCGAAGCTCA |
| | | | | GCCCTCTTGCCT GCCCCGGAGCTGTCCCGGG |
| 3710 | ITPR3 | HSX2_01015842 | 352 | CTGGGCACCTGCCCCACCAAGGAGGACAAGGAGGCCTTTGCCATCGTGTCAG |
| | | | | TGCCCGTGTCTG |
| | | | | AGATCCGAGACCTGGACTTTGCCAATGACGCCAGCTCCATGCTGGCCAGTGCC |
| | | | | GTGGAGAAACT CAACGAGGGCTTCATCAGCCAGAATGACCGCAG |
| 3751 | KCND2 | HSX2_01017841 | 353 | CCGATCCAGTTTAAATGCCAAAATGGAAGAGTGTGTTAAACTAAACTGTGAAC |
| | | | | AACCTTATGTGA |
| | | | | CTACAGCAATAATAAGCATCCCAACACCTCCAGTAACCACACCAGAAGGAGAC |
| | | | | GATAGGCCAGA |
| | | | | ATCCCCTGAGTACTCAGGAGGAAATATTGTCAGAGTTTCTGCTTTGTAAGACA |
| | | | | ATTGGAATAAGG |
| | | | | TCTAAGAGAATTCGAGCCCTGGCTGTGAAAAGAATCTCAACATAGAAGAAAG |
| | | | | AAGAAACAATAA |
| | | | | ATATTCTGCAGATTAATGCAGCAAAGAAAGAAGGTTGGTAGTGAAACACAAA |
| | | | | GCTTCCAATCTTA |
| | | | | AGGATGTGAATAAAACCACCAAATGGCATTTCTAGACAGTTTGACCTGTTATA |
| | | | | CAGAGTAATATTC |
| | | | | TGTGGCCCTTTGACTTTGTGAATGAGCACAATGAAATGCCGCCTACTGA |
| 8645 | KCNK5 | HSX2_01350094 | 354 | GTGTGAACCCCAGCGCCAACTACCACGCCCTGTACCGCTACTTCGTGGAGCTC |
| | | | | TGGATCTACTTG |
| | | | | GGGCTGGCCTGGCTGTCCCTTTTTGTCAACTGGAAGGTGAGCATGTTTGTGGA |
| | | | | AGTCCACAAAGC |
| | | | | CATTAAGAAGCGGCGGCGGCGACGGAAGGAGTCCTTTGAGAGCTCCCCACAC |
| | | | | TCCCGGAAGGCC |
| | | | | CTGCAGGTGAAGGGGAGCACAGCCTCCAAGGACGTCAACATCTTCAGCTTTCT |
| | | | | TTCCAAGAAGGA |
| | | | | AGAGACCTACAACGACCTCATCAAGCAGATCGGGAAGAAGGCCATGAAGACA |
| | | | | AGCGGGGGTGG |
| | | | | GGAGACGGGCCCGGGCCCAGGGCTGGGGCCTCAAGGCGGTGGGCTCCCAGC |
| | | | | ACTGCCCCCTTC |
| | | | | CCTGGTGCCCCTGGTAGTCTACTCCAAGAACCGGGTGCCCACCTTGGAAGAG |
| | | | | GTGTCACAGACA |
| | | | | CTGAGGAGCAAAGGCCACGTATCAAGGTCCCCAGATGAGGAGGCTGTGGCA |
| | | | | CGGGCCCCTGAA |
| | | | | GACAGCTCCCCTGCCCCCGAGGTGTTCATGAACCAGCTGGACCGCATCAGCGA |
| | | | | GGAATGCGAGC |
| | | | | CATGGGACGCCCAGGACTACCACCCACTCATCTTCCAGGACGCCAGCATCACC |
| | | | | TTCGTGAACACG |
| | | | | GAGGCTGGCCTCTCAGACGAGGAGACCTCCAAGTCCTCGCTAGAGGACAACT |
| | | | | TGGCAGGGGAG |
| | | | | GAGAGCCCCCAGCAGGGGGCTGAAGCCAAGGCGCCCCTGAACATGGGCGAG |
| | | | | TTCCCCTCCTCCT |
| | | | | CCGAGTCCACCTTCACCAGCACTGAGTCTGAGCTCTCTGTGCCTTACGAACAG |
| | | | | CTGATGAATGAG |
| | | | | TACAACAAGGCTAACAGCCCCAAGGGCACATGAGGCAGGGCCGGCTCCCCAC |
| | | | | CCCACCTTTGAT |
| | | | | GGCCTCTTCCCCCCTCACCCTAGGGTGTCCCGAGATGACCGGGACGCCTGGCC |
| | | | | CCTGGTGGGGG |
| | | | | GGCAGCCTCGGAACTGGGAGTGGGGGGCCAGGGGCCTTCCTAACCTTCCATC |
| | | | | ATCCTC |
| 8909 | ENDOU | HSX2_01371797 | 355 | TGGGATTCTTACCCCGATGTGCTGGCAATGCAGTTCAACTGGGACGGCTACTA |
| | | | | TAAGGAAGTGG |
| | | | | GCTCTGCTTTCATCGGCAGCAGCCCTGAGTTTGAGTTTGCACTCTACTCCCTGT |
| | | | | GCTTCATCGCCA GGCCAGGCAAAGT |
| 23081 | KDM4C | HSX2_02542306 | 356 | AAATGCATCTTCTGCAGACACCGGGTTAAGAGGGTCTCTGGAGCCTGCATCCA |
| | | | | GTGTTCCTACGG |
| | | | | TCGCTGCCCGGCCTCCTTCCATGTCACTTGTGCCCATGCTGCTGGGGTACTGAT |
| | | | | GGAGCCTGATG |
| | | | | ACTGGCCTTATGTGGTGAACATTACATGCTTTCGACATAAGGTCAACCCCAAC |
| | | | | GTG |
| 90231 | KIAA2013 | HSX2_02132945 | 357 | ATCCCAGTGTCTGAGTGAACTAACAGTCCTGCTTTCAGCCACCATTTGCACAAG |
| | | | | ACACCCAGCACT |
| | | | | GAAAGTCCCGCTGCCAGGAGCAAGGGATCCTTTGGAAGCACCCGCCCTTTGT |
| | | | | GCCTTGTTGGGG |
| | | | | GAAACCGGTGACGCAGAAGTGAGTGTGGATACACCAGAGTTTGCATTGGAAG |
| | | | | GAATGAGTGTCA |
| | | | | CGTGGGGAGGGAAGGGGCCAGTGGACCTTTTGTAAGCTTTCCACTCAATAAA |
| | | | | ATGAACCTGTAT GGCAA |
| 55220 | KLHDC8A | HSX2_01840918 | 358 | GGGGATGGCTGAAGATGGAACGATCGTTCTTCCTCAAGAAGCGGCGGGCAG |
| | | | | ATTTTGTGGCTGG CTCTCTGAGTGGACGGGTCATAGTGGCTGGGGGACTTG |
| 5655 | KLK10 | HSX2_02514669 | 359 | ACCAGCGGCAGACCACAGGCAGGGCAGAGGCACGTCTGGGTCCCCTCCCTCC |
| | | | | TTCCTATCGGCGACTCCCAG |
| 5650 | KLK7 | HSX2_01154792 | 360 | TGAGTACACCGTGCACCTGGGCAGTGATACGCTGGGCGACAGGAGAGCTCAG |
| | | | | AGGATCAAGGC |
| | | | | CTCGAAGTCATTCCGCCACCCCGGCTACTCCACACAGACCCATGTTAATGACCT |
| | | | | CATGCTCGTGAA |
| | | | | GCTCAATAGCCAGGCCAGGCTGTCATCCATGGTGAAGAAAGTCAGGCTGCCC |
| | | | | TCCCGCTGCGAA |
| | | | | CCCCCTGGAACCACCTGTACTGTCTCCGGCTGGGGCACTACCACGAGCCCAGA |
| | | | | TG |
| 5650 | KLK7 | HSX2_01154804 | 361 | GGTGACTCAGGGGGACCGTTGGTGTGCAGAGGTACCCTGCAAGGTCTGGTGT |
| | | | | CCTGGGGAACTT |
| | | | | TCCCTTGCGGCCAACCCAATGACCCAGGAGTCTACACTCAAGTGTGCAAGTTC |
| | | | | ACCAAGTGGATA |
| | | | | AATGACACCATGAAAAAGCATCGCTAACGCCACACTGAGTTAATTAACTGTGT |
| | | | | GCTTCCAACAGA |
| | | | | AAATGCACAGGAGTGAGGACGCCGATGACCTATGAAGTCAAATTTGACTTTA |
| | | | | CCTTTCCTCAAAG |
| | | | | ATATATTTAAACCAACCTCATGCCCTGTTGATAAACCAATCAAATTGGTAAAGA |
| | | | | CCTAAAACCAAA |
| | | | | ACAAATAAAGAAACACAAAACCCTCAGTGCTGGAGAAGAGTCAGTGAGACCA |
| | | | | GCACTCTCAAAC ACTGGAA |
| 79875 | THSD4 | HSX2_02028559 | 362 | AACTACTACAAGACCGCCTGCTGTGCCTCCTGCACCCGTGTGGCCAACAGGCA |
| | | | | GACGGGCTTCCT |
| | | | | GGGGAGCAGATAACACTCCTGCACCCCCATCAGTAGGGCAGCATCACTGCCTT |
| | | | | CCCGGGGGCTT |
| | | | | CAGCAGTGCGCCTGGCTGGCTGCTGCTCCACCACGGGCCCCCTGGCCCAGGC |
| | | | | GCTGCCAACCAA |
| | | | | CTTAGTCACCACCCCTGCCTCCGGTGAATGCACCCCGTGGTACCCAGGGGCTT |
| | | | | TTTACACAAGAT |
| | | | | GTTTGAAAGCCACAGTCAGTCCTTTAAGCATCACCATGTACTGATGATCCCCTC |
| | | | | CTTGGACCTGGC |
| | | | | ATCTGCTAATGGTGCCCTTTGAAAGTCAAGCAGTGGGAAGTACATGGAGCTCT |
| | | | | CAGCCCTGCTCC |
| | | | | CATCTGGCACCTTCAAGTCAGCAGATGGGCCACTGACTGAGCACTGCCCCGTC |
| | | | | CCTGGTGCTACT |
| | | | | GGTCTTTCTAAACTTAGCACCCTGGAGAGTCCAAGGAGGCAGCGCCCCCAACC |
| | | | | CAGCGCCCCACT |
| | | | | AAGCCTTGCTGACACGCGTGCATCCCTCTGTGACCTCAGCCCAGATGTGCCTG |
| | | | | TTTTCATTCTCAA |
| | | | | AGACATTAGACTGTTTTCCTGCCCTATGACACAGATAGCTCACATGAATATTGT |
| | | | | GCTTTATTTAGC |
| | | | | AGGTGTACTCACAGATACTAGCTCCTTAGCAGCTCACAACATCCCAGAATGGG |
| | | | | AGGCAGGGGGT |
| | | | | GACTCATTATCCCCATTTTACTGACAGGGAAACTGAGGCTCAACTTAAGTAATT |
| | | | | GACCTGCCAGGT |
| | | | | ATATTCACCCATCCAGTGGAAGAGCTGAGTCCCCGCCCCAGTCATCTACCAGT |
| | | | | ATCCAGCCTGGG |
| | | | | GCCTGTACTTAGATGTGAAAGGTGCTGCTTCATTTCTGACCAAGAGACTGAGA |
| | | | | AGTTTCCCAGAA |
| | | | | TGCAAACAAAGCCCAGGCCCCTGAAATCTTTCCGGTCAAGCCTTTATCCCAGC |
| | | | | ACTCAGTTGTTTT |
| | | | | GGATGTCTGTTCCTACTTGCCCTTACCCCCAAAGTTACAGATCCTAGTTACAGG |
| | | | | ACTCTGCCAGCT |
| | | | | TTGTTAAACTGTCCGTGAGACAAGAAAGCCATTGGGGAAACCAGGTGATTGC |
| | | | | CTGAAATTCTTAC |
| | | | | TCCGTTCCAAGTGCTGTTCCTCCCAGGAAATCAAAGGCCAGGGTCCTTATGGC |
| | | | | CGTGGAGCCTTC |
| | | | | CCGACCACAGAGCCAACTTGTGAAGCACACAGCTCTGCAGCCTGGGCTCTGCC |
| | | | | CTGCCTCAGCCG |
| | | | | CCTCCCCCACGCTCTTCACCACGTTCCTGGAGAGTCCGGCCAACCTGTCCCAGC |
| | | | | CGAAACACTGCT |
| | | | | GTATTAGAAAAAGTCTCTTTCTGGTCTTTCTGGTTTTGTTTATGAATTTCCCTCT |
| | | | | GTGGCCACAAAT |
| | | | | TCCTCCCCTCCCCCATGACTCACAGTCCATATGGCCCACCCCCAGACTTGAGCA |
| | | | | CCAAGCTCTGCA |
| | | | | TTAATGCAGTTGGCCTGCGACAAGGAGCTGTGGACCCTTCCCCATCTCTTCCA |
| | | | | ATTCACTTTCCCC |
| | | | | AACTATCCAGTTCCAGAGGCCGCAGGCCTGGAAGGATGCAGTGCATATTGAA |
| | | | | AGGTGGACCCTC |
| | | | | TGAAAACAGTTAAGAGGAATATATGTATGTTTTACCCATTAAGAAAAAATGGC |
| | | | | AAGCTAAACAAA |
| | | | | TGTTAAACTTACAGAAAATTTGTCTTATGGTCCTGAGCATATTTCCCTTTTAGA |
| | | | | GCAAGCCTGGATT |
| | | | | CTTAGCAAAGTGTTTCCCCCATTTGCTCTTTTAGCTGACAAATCTGCCACTGTG |
| | | | | ATGATGGTTTGCA |
| | | | | GCTTTTGGAAGCAGTATGGCAACCTGGCCTGACATGCTCTTTAGGCTTCCACT |
| | | | | AACCTGGGGCTTT |
| | | | | CAGAAATTCTATTTGGCCTTTCTGTGGGTAGCTTTCCAGCTTCTCTTCTAGGGA |
| | | | | GCCCCAGGCATC |
| | | | | ATTTCCCAAAAGCATCCCCATCTCCTGATTCTCTTGGAACTCCTACAGATAAGC |
| | | | | ATCCTGGCAGAG |
| | | | | GCCCAGGCTCCCAAACCGACAAAGTGAAAAGAGACCAGAGAGGCCAAGCAT |
| | | | | ATTGACTGGTGCT |
| | | | | GTTCAGGGCCTGCTCTTTTCCACTCACCACTTGTTTTGCTGCTTGTCACGAGGA |
| | | | | GAGTTGTTCCTG |
| | | | | TATGTGGCTGCTCTCAGATCTTTCCAAGCAAGCCAGTCATTTGAAGAGGTTTTC |
| | | | | TTTTCATGCTGG |
| | | | | AGGGCAGGCTAAGATCAATGAGTGGAAGAGAGAAAGGCTGTTTTAGCTCAA |
| | | | | GTTAAAGGAACAC |
| 23355 | VPS8 | HSX2_01628191 | 363 | GAGAGTGATGGGAGTGAGAACCTGTGAATCTAGATGTCTGTTCAGTGGTTCC |
| | | | | AAGGGTGAAGTC |
| | | | | TGCTGTATTGAGCCTCTGCATTCTAAGCCTGAGTTGAAAGATCATCCCATCACA |
| | | | | CAGTTTTCATTAT TGGCCATGGCATCCTTGACAAAA |
| 23355 | VPS8 | HSX2_01628350 | 364 | AGAGTGGATCATCTCCTGAAACAAGATTGTCTTACAGAAGCGTTGGCTCTTGC |
| | | | | GTGGTCTTTCCAT GAAGGAAAAGCAAAAGCAGTAGTGG |
| 7462 | LAT2 | HSX2_01300472 | 365 | GAGCCAAGGCAAAGAGGGACCACTGTGCTCATGGACCCATCGCTGCCTTCCA |
| | | | | AGGACCATTTCCC |
| | | | | AGAGCTACTCAACTTTTAAGCCCCTGCCATGGTTGCTCCTGGAAGGAGAACCA |
| | | | | GCCACCCTGAG |
| 286256 | LCN12 | HSX2_02348587 | 366 | AGGCCAGCACTGTGACACATGGTCTTATGTGCTGATACCGGCAGCCCAGCCTG |
| | | | | GGCAGTTCACTG TGGACCACGGTGTGG |
| 3977 | LIFR | HSX2_01034760 | 367 | GGGCTCCTCATGATTTGAAGTGTGTAACTAACAATTTGCAAGTGTGGAACTGT |
| | | | | TCTTGGAAAGCA |
| | | | | CCCTCTGGAACAGGCCGTGGTACTGATTATGAAGTTTGCATTGAAAACAG |
| 200879 | LIPH | HSX2_02286232 | 368 | ATTTACATGATCGGAGTAAGTCTAGGAGCCCACATATCTGGGTTTGTTGGAGA |
| | | | | GATGTACGATGG ATGGCTGGGGAGAATTACAG |
| 200879 | LIPH | HSX2_02286275 | 369 | GATTTCAGTATTTTAAATGTGACCACCAGAGGTCTGTATACCTGTACCTGTCTT |
| | | | | CCCTGAGAGAGA |
| | | | | GCTGCACCATCACTGCGTATCCCTGTGACTCCTACCAGGATTATAGGAATGGC |
| | | | | AAGTGTGTCAGC TGCGGCACGTCACAAAAAGAGTCCTGTCCCCTTCTGG |
| 200879 | LIPH | HSX2_02286297 | 370 | GCTATTATGCTGATAATTGGAAAGACCATCTAAGGGGGAAAGATCCTCCAATG |
| | | | | ACGAAGGCATTC TTTGACACAGCTGAGGAGAGCCCATTCTGCA |
| 200879 | LIPH | HSX2_02286330 | 371 | GCCTCAGCTGTGTCGGTATGATCTTGTCCTGATGGAAAACGTTGAAACAGTCT |
| | | | | TCCAACCTATTCT |
| | | | | TTGCCCAGAGTTGCAGTTGTAACTGTTGCCAGGACACATGGCCATAAATAATA |
| | | | | GAAAGAAAGCTA |
| | | | | CAACCACAGGCTGTTTGAAAGCTTCACCTCACCTTTCTGCAAGGCAGAAAAAG |
| | | | | TATGAAAAAAAC |
| | | | | CAAGGCTTTTTTCAGTAGCGTCCTATGGATGTCACATTGTACATCAAACAACCT |
| | | | | TGTGATTATAAA |
| | | | | ACGATCCTGGGAAGGAGCCCCTAACTAGGGCAAGTCAGAAATAGCCAGGCTC |
| | | | | GCAGCAGCGCA |
| | | | | GCGCTGTGTCTGCTGTGTCCTGGGGCCTCCCTTGTTCCGACCTGTCAATTCTGC |
| | | | | TGCCTGTCACGC |
| | | | | GGGTGGTTCTGCCCATCGCGGCTGCGGGTCAAGCATCTTCAAGGGAAGGACG |
| | | | | GACTGGAGGCCT |
| | | | | CACCGTGGACTCAACTCTGCATTCTCCGTGCCACATTCCTCCAGTTCCCACACG |
| | | | | TAGAAGGGAAC |
| | | | | GAAACTGACGTCTACCTCATGGGGCTGCTGTGTGGGTTTGGGAGGCAAAAAT |
| | | | | CTATGAAGGGTT |
| | | | | TTTTGAAATCCCATAGGTGCCACATCTATGAGATGTTTGATAAATGTGAATATG |
| | | | | CTTTTACATTTG |
| | | | | GGCTTATCTAATTTGCAATAAGAGAGCCTCTCTCTATCAACACCAGCTTCTCTC |
| | | | | TCGGGCTGTTTG |
| | | | | CTCAGGGAAGGCAAGAAAGCCACGTGCTGGCCCTCTGCCTTCTCTAAAGTGCT |
| | | | | GTTGGAGCATG |
| | | | | GAGGAGCTGGAGGAGATGGGGATGGACTGACAGCTAAGAGGGCGGCTGCT |
| | | | | GGGACTAGATAG |
| | | | | TGGATGAAGAAAGAAGGACGAGGAAGCCGTGGGGCAGCCTCTTCACATGGG |
| | | | | GACAGGGGATG |
| | | | | GAGCATGAGGCAAGGGAAGGAAAAGCAGAGCTTATTTTTCACCTAAGGTGGA |
| | | | | GAAGGATCACTT |
| | | | | TACAGGCAACGCTCATTTTAAGCAACCCTTAAGAAATGTTTATGTTTCTTTATT |
| | | | | ACCAATGTAATCT ATGATTATTGAAGGAAATTTAGAAAATGCGTAGATACAA |
| 200879 | LIPH | HSX2_02580660 | 372 | GCAGAAGGAGCTTCTCTTGATGACATTTACATGATCGGAGTAAGTCTAGGAGC |
| | | | | CCACATATCTGG |
| | | | | GTTTGTTGGAGAGATGTACGATGGATGGCTGGGGAGAATTACAG |
| 4000 | LMNA | HSX2_02507428 | 373 | GTGAGTGGTAGCCGCCGCTGAGGCCGAGCCTGCACTGGGGCCACCCAGCCAG |
| | | | | GCCTGGGGGCA |
| | | | | GCCTCTCCCCAGCCTCCCCGTGCCAAAAATCTTTTCATTAAAGAATGTTTTGGA |
| | | | | ACTTT |
| 55164 | SHQ1 | HSX2_01835838 | 374 | GAATTTTTACCATTCGCCTGCCCAAAGAAACCCCTGGCCAGCATTTTGAGGGG |
| | | | | CTGAACATGTTA |
| | | | | ACTGCTCTTCTGGCACCAAGAAAATCCAGGACAGCAAAACCACTTGTGGAAG |
| 55164 | SHQ1 | HSX2_02556484 | 375 | GAATTTTTACCATTCGCCTGCCCAAAGAAACCCCTGGCCAGCATTTTGAGGGG |
| | | | | CTGAACATGTTA |
| | | | | ACTGCTCTTCTGGCACCAAGAAAATCCAGGACAGCAAAACCACTTGTGGAAGA |
| | | | | AATAG |
| 23211 | ZC3H4 | HSX2_01609614 | 376 | GAACTATGGAGGCCGCGCCCGGGACCCCCCCGCCGCCGCCATCAGAGTCGCC |
| | | | | GCCGCCGCCATC |
| | | | | GCCGCCGCCGCCATCAACGCCTTCGCCTCCTCCGTGTTCCCCCGACGCCCGCCC |
| | | | | GGCCACCCCGC ACCTCCTCCACCACCGCCTCCCGCTCCCTGACGACAG |
| 100509091 | LOC1005090 | 9HSX2_02468543 | 377 | ACAAGTGCCCTTGAATTCCATCTGTGACATGCGCGTGGCTCAGGACTCCAACC |
| | | | | TTACCTCCACACA |
| | | | | CCTGGAGAACAACCTCATTGACCGGCGCCGCATCCCGCCCACTGCCTTCTCCT |
| | | | | GCACCCGAGCCT |
| | | | | ATCACAGCGTGGTCCTCCAGCCCCAGCGGCGGGGGGAGGAGGGCTCCTAGTC |
| | | | | CTGCCTGCCGCC |
| | | | | TTCCTCCAGCACACACTCTGCTTCTCGAGGGGGTGGAGGGGCAGGCTGAGGT |
| | | | | GTGCTTGGCCTTT |
| | | | | GCACCACTGGGAGAAAGGTCTAGAAGACCCCACTGCTTCCAGTGCACCCACTT |
| | | | | CCCATTGACTGG |
| | | | | TGAGGCCGCTTCACTTAGCAAATGGCCCAGGGGCGAAAGGTGGCACTGGTTT |
| | | | | GTACTTGAACAT |
| | | | | AATAAATGCAGCTTGGGCAGGCTCACCAGCCAGCCTCCCTCCCTCCAGGAGCT |
| | | | | ACGCTGCATGTC CTGGC |
| 9697 | TRAM2 | HSX2_01428829 | 378 | GTTCAGTGCCTGGGCTGCTGTTTTTGGGGTTACCCGCCTCTTCATCCTCACCCT |
| | | | | TGCCGTGCTGGC |
| | | | | CATTGGCTTTGGACTGGCTCGCATGGAAAACCAGGCATTTGATCCCGAGAAA |
| | | | | GGGAACTTCAACA CTTTGTTTTGCAG |
| 23248 | RPRD2 | HSX2_01614852 | 379 | TCGAGGACAGGAAGGAAAAACCTGCAGAGAAGTCAGCTGTATCCACTTCTGT |
| | | | | ACCTACAAAGCC |
| | | | | AACAGAAAATATCTCAAAGGCCTCTTCATGTACCCCAGTGCCTGTGACCATGA |
| | | | | CAGCAACTCCAC |
| | | | | CTCTTCCAAAGCCTGTGAATACTTCTCTTTCCCCTTCCCCAGCATTGGCTTTGCC |
| | | | | AAACCTGGCTAA |
| | | | | TGTGGATCTGGCAAAGATCAGTTCCATCCTTAGCAGTTTAACATCAGTCATGA |
| | | | | AAAATACTG |
| 23248 | RPRD2 | HSX2_01614881 | 380 | GGGTCAGTCCTGCATCAAGACCTTCTCCAGGAACGCCCACCAGCCCCAGCAAC |
| | | | | CTCACCAGTGGC |
| | | | | CTGAAAACACCTGCACCTGCCACGACAACATCTCACAACCCTCTGGCAAATATC |
| | | | | CTCTCCAAGGTG |
| | | | | GAGATCACCCCAGAGAGCATTCTGTCTGCACTTTCCAAAACCCAGACACAGTC |
| | | | | AGCCCCTGCACT GCAAG |
| 23248 | RPRD2 | HSX2_01614918 | 381 | CCAAAAGCTTCAACTATTCTCCTAACTCATCAACTTCTGAAGTCTCTTCAACTTC |
| | | | | AGCCAGCAAGGC |
| | | | | CTCAATTGGGCAAAGCCCAGGGCTCCCAAGCACTACTTTTAAACTACCTTCCAA |
| | | | | CTCTTTGGGGTT |
| | | | | TACAGCTACCCACAATACTAGCCCTGCTGCCCCACCTACTGAAGTTACCATCTG |
| | | | | CCAATCTTCAGA |
| | | | | GGTCTCCAAGCCAAAGCTGGAGTCAGAGTCCACCTCCCCAAGCCTGGAAATG |
| | | | | AAGATTCACAACT |
| | | | | TCTTAAAAGGTAATCCTGGTTTCAGTGGCTTAAACTTAAACATCCCAATCCTGA |
| | | | | GCAGTTTGGGGT |
| | | | | CCAGCGCCCCATCAGAGAGCCATCCCTCAGACTTCCAGCGTGGCCCTACTAGC |
| | | | | ACCTCAATCGAC |
| | | | | AACATTGATGGAACCCCTGTACGGGATGAACGGAGTGGGACACCCACCCAGG |
| | | | | ATGAGATGATGG |
| | | | | ACAAGCCCACATCCAGCAGTGTAGATACTATGTCCCTGCTTTCTAAGATCATTA |
| | | | | GCCCTGGTTCCT |
| | | | | CAACACCCAGCAGTACAAGATCACCACCCCCTGGGAGAGATGAAAGCTACCC |
| | | | | CCGAGAGCTCTC |
| | | | | CAATTCTGTATCTACATATCGACCCTTTGGTCTGGGCAGTGAATCTCCCTATAA |
| | | | | GCAGCCTTCTGA |
| | | | | TGGAATGGAGAGACCATCTTCCCTGATGGACTCTTCACAGGAAAAGTTCTACC |
| | | | | CAGATACTTCTTT |
| | | | | CCAAGAAGATGAGGATTACCGAGATTTTGAGTATTCAGGGCCTCCACCCTCTG |
| | | | | CCATGATGAACC |
| | | | | TAGAGAAGAAACCAGCCAAATCTATCCTGAAATCAAGCAAGCTGTCTGATACC |
| | | | | ACCGAGTACCAG |
| | | | | CCAATTCTGTCCAGTTATAGCCACAGAGCCCAAGAATTTGGGGTAAAGTCTGC |
| | | | | CTTCCCTCCATCT |
| | | | | GTAAGGGCCCTCCTGGACTCTAGTGAGAACTGTGACCGTCTCTCATCTTCCCCT |
| | | | | GGGCTATTTGGT |
| | | | | GCCTTCAGCGTAAGAGGGAATGAACCTGGGTCTGACCGGTCACCATCACCGA |
| 23248 | RPRD2 | HSX2_01614922 | 382 | GTAAGAATGATTCATTTTTCACCCCTGACTCCAACCACAATAGCTTGTCTCAAT |
| | | | | CTACCACTGGGC |
| | | | | ATCTCAGTTTGCCACAGAAGCAGTACCCAGACTCTCCTCACCCAGTCCCACATC |
| | | | | GTTCCCTTTTCTC TCCGCAG |
| 23248 | RPRD2 | HSX2_01614923 | 383 | AACACCCTTGCCGCTCCCACGGGTCACCCACCCACGTCAGGCGTGGAGAAAGT |
| | | | | CCTGGCCTCCAC |
| | | | | CATTTCCACCACGTCGACGATTGAATTTAAGAATATGCTTAAAAACGCCTCACG |
| | | | | TAAGCCCTCAGA |
| | | | | TGATAAGCATTTTGGCCAGGCTCCCAGCAAGGGCACTCCAAGTGATGGTGTCA |
| | | | | GTCTCTCAAACC |
| | | | | TCACCCAACCCAGCTTGACCGCCACTGATCAGCAGCAACAAGAAGAGCACTAC |
| | | | | CGCATAGAAACC |
| | | | | CGCGTCTCCTCCTCCTGCTTAGACTTGCCTGATAGCACAGAAGAAAAGGGGGC |
| | | | | CCCTATAGAAAC |
| | | | | CTTGGGTTATCACAGTGCATCCAATAGGAGGATGTCAGGGGAGCCGATCCAG |
| | | | | ACCGTAGAGTCC |
| | | | | ATCCGAGTTCCTGGGAAGGGAAATAGAGGACATGGGCGTGAGGCTTCAAGG |
| | | | | GTGGGTTGGTTT |
| | | | | GATCTGAGCACATCAGGTAGCTCTTTTGACAATGGCCCTTCAAGTGCCTCTGA |
| | | | | GTTGGCATCCCTT |
| | | | | GGGGGTGGGGGCAGCGGAGGCCTCACTGGCTTTAAAACAGCACCATACAAG |
| | | | | GAACGGGCACCT |
| | | | | CAATTTCAGGAGAGTGTCGGCAGCTTTCGTTCCAACAGTTTCAACTCAACATTT |
| | | | | GAGCATCATCTT |
| | | | | CCCCCATCCCCCTTGGAACATGGGACACCCTTCCAGAGAGAGCCAGTGGGGC |
| | | | | CATCATCTGCCCC |
| | | | | ACCTGTCCCTCCTAAGGATCATGGTGGTATCTTCTCTCGAGATGCACCCACTCA |
| | | | | TCTACCCTCTGT |
| | | | | GGATCTTTCGAACCCCTTCACAAAGGAGGCAGCCCTGGCCCATGCTGCCCCAC |
| | | | | CCCCTCCTCCTG |
| | | | | GAGAGCACAGTGGAATTCCTTTCCCTACCCCACCTCCTCCTCCCCCTCCTGGGG |
| | | | | AACATAGCAGCA |
| | | | | GTGGTGGGAGTGGTGTCCCCTTTTCTACTCCACCCCCTCCTCCACCCCCTGTTG |
| | | | | ACCACTCTGGAG |
| | | | | TTGTACCCTTCCCAGCCCCACCACTGGCAGAGCACGGAGTGGCAGGGGCTGT |
| | | | | GGCAGTATTTCCC |
| | | | | AAGGACCATAGTTCCCTCCTTCAAGGGACCCTGGCTGAGCATTTTGGGGTACT |
| | | | | CCCAGGACCCAG |
| | | | | GGACCACGGGGGCCCCACCCAACGGGACCTCAACGGCCCTGGCCTTAGCCGT |
| | | | | GTACGAGAGAG |
| | | | | CCTCACCCTACCCTCCCATTCTCTGGAACACCTGGGCCCACCCCATGGAGGAG |
| | | | | GAGGTGGGGGA |
| | | | | GGCAGCAACAGCAGCAGTGGCCCCCCCTTGGGTCCCTCACACAGAGACACCA |
| | | | | TCAGCCGGAGTG |
| | | | | GTATAATCTTACGGAGTCCCCGGCCAGACTTTCGGCCTAGGGAACCTTTTCTCA |
| | | | | GCAGAGACCCA |
| | | | | TTTCACAGTTTAAAGAGACCCAGGCCACCTTTTGCTAGGGGCCCTCCGTTCTTT |
| | | | | GCACCAAAACGC |
| 84561 | SLC12A8 | HSX2_02103940 | 384 | ATTTCTTCCTGAAGTCCAGGCTCCAAGAACAAGATGTCTGGAGAAGATCCACT |
| | | | | TCTTTCTATACCC ACATGTGCAACCCCTGGGTCTCCCTGTTGGGG |
| 54532 | USP53 | HSX2_02554222 | 385 | ATTGGAACTAGATGGAAAGATGTTGTCTCCAAATGCATTCGATGCCACTTTCA |
| | | | | GCCACTACTTTTG |
| | | | | TTTTATGCAAACCCAGATGGCACAGCAGTTTCTACTGAGGATGCACTCAGGCA |
| | | | | GGTCATCAGCTG GTCACATTACAAATCTGTTGCAGAAAATATGG |
| 285180 | RUFY4 | HSX2_02341096 | 386 | GTCACGCTGCTCCAGTTCCACCCAAACCCAGGGAAGGAGACCCAGAAAAAAC |
| | | | | AAAGATGCCCCA AAGAAG |
| 164832 | LONRF2 | HSX2_02269570 | 387 | GTAATGTGTGAAGTGCTGTTTTCAGCTACAGCAAATGTGCATGAAAATTTAAC |
| | | | | ATCTTCCATCCAA |
| | | | | AGCAGATTAAAGGCTCAGGGTCACAGCCACATGAATGCCCAGGCTCTGCTGG |
| | | | | AAGAAGGTGATG CAGGGAGCTCGGAG |
| 164832 | LONRF2 | HSX2_02269581 | 388 | AATTCATCTGAGAAATCTGATATGCTTGGAAATACCAATTCTTCAGTTTTGTAT |
| | | | | TTTATACTGGGTC |
| | | | | TACACTTTGAAGAGGATAAAAAGGCGTTAGAAAGCATCCTTCCAACAGCACCC |
| | | | | AGCGCTGGCTTA |
| | | | | AAGAGACAGTTTCCGGATGACGTGGAGGATGCACCTGACCTGAACGCCCCTG |
| | | | | GAAAAATTCCCA AGAAAG |
| 164832 | LONRF2 | HSX2_02269589 | 389 | ATCTCTCACTTCAAAGGAGCCCAAACTCTGAGACAGAAGAAAGTCAGGGGCT |
| | | | | CTCGCTTGATGTA ACTGACTTTGAGTGTGCCCTCTGCATGAG |
| 164832 | LONRF2 | HSX2_02269599 | 390 | ATTGCTCTTTGAACCTGTCACTACGCCCTGTGGACACACATTTTGCCTAAAATG |
| | | | | CCTTGAGCGCTG |
| | | | | CCTTGACCACGCCCCACACTGTCCTTTGTGCAAAGACAAACTTTCGGAA |
| 164832 | LONRF2 | HSX2_02269630 | 391 | GCTTTCAGAGTATGGATGCATGCTGGAGATTAAGGACGTGAGAACGTTTCCT |
| | | | | GATGGAAGTTCTG |
| | | | | TTGTAGACGCGATTGGCATCAGTCGGTTCCGAGTGCTAAGCCACCGCCACAGA |
| | | | | GATGGCTATAAC ACAGCGGACATTGAATATCTTGAAGATGAAAAG |
| 164832 | LONRF2 | HSX2_02269640 | 392 | GTGGAGGGTCCAGAGTATGAAGAACTTGCCGCTCTCCACGATTCTGTGCATCA |
| | | | | ACAGTCTGTTTC |
| | | | | CTGGTTCGCGTCTCTCCAGGATCGCATGAAAGAACAAATTTTAAGTCATTTTG |
| | | | | GGGTAATGCCAGACAGAGAACCTGAGCCTCAG |
| 164832 | LONRF2 | HSX2_02269652 | 393 | ATTGAGTCCTCCTCAAAAATGACAATTCTGTGTCTGGTGGGATCTGACCTTGTG |
| | | | | TGAGGTTAGCCT |
| | | | | GAAGTCTGAATGGAGCCCATAGTTGGAAAACAACCTAAGAAAATCTCTTAGA |
| | | | | AGCAGGTGCTTG |
| | | | | GGGAATGCAGTTCACTGACAGCACAGGACCCTGCAGATGGTTTACATGTGGT |
| | | | | TTGGGTTTCACGA |
| | | | | GAAAGAAGGATTCACTTCCCAGTCAGCATCTGGCTCTGCCAGATGGTAAAGG |
| | | | | CGTGCTTTAGTGT |
| | | | | GTAGACAATATGGGGGAACCACGTTTTTATCTGGAAGTGGATTTCTTAGAACA |
| | | | | CAGGCTAACCAA |
| | | | | AACTACGCTTAGGCTTTGCGTGTTGCTGTGAAGTTGTCTGTGAAATCGAATAA |
| | | | | TCACACCATTGTT |
| | | | | CAGTGCAGGAGCCCAAACTAGTCCTTACCCAAGAAGTAGTAGCCTCTGGATA |
| | | | | GAACTGTGTTTAA |
| | | | | TGTCCTGTTGTAGTCCCAGGTGTTGTAAATTGCATGTTGTAATCAAACGAATGT |
| | | | | CAAAACATAAGA |
| | | | | AAGTATACCTTGGATATAGAAAAACCTGAGAACAGTATCATTCACTTGAGGAT |
| | | | | ATATATATATATA |
| | | | | TTTACACACAATAAAGTGAGTTAAAATTGTATATGCATTGGGATGTCAAACAT |
| | | | | AAAACCACCAAGT |
| | | | | GCAAAGAATGCTTTGAAAGTAGAACCTTGTCTCATTGATCAGTGGTTACTAAG |
| | | | | CATTTAGGAAAC |
| | | | | AGTCATCTTTTTCTATTGGGATTTGCATTAGAAATTAGCCCCATCAGTAAATTTC |
| | | | | AGTTTACTATCC |
| | | | | ATTTATTAATATACAAAAACATTAAAAAATCTGCCATGCCATGGTAATCTATCT |
| | | | | ATTCCAGGAAAAT |
| | | | | AGATCAGAATATATGAAGGGAGGATGGCCGGTATGGGATGTAAATATTCAGT |
| | | | | AAGTTTTTCTCCA |
| | | | | ACACCAACCTAAAATTATCTTTATTTTTTTGAAAGAAAGTTTTACGGTGATCAG |
| | | | | ACCAGGTGTTATC |
| | | | | TGTTAAAGGACAGTAGTATGACTTCCATCCTGTAATGCACGTTGGTGATATTG |
| | | | | ATTGGAATAACTG |
| | | | | AATCTGAGATACTCATATGTTCTCTCTGGCCCAACCAGCTGGTGCATTTGGTTG |
| | | | | GCTTCCTCGTCT |
| | | | | GTAAGGTGGACATTCTTAACCAGAGGGTCACGACCACATCACAGCTGCAAGTT |
| | | | | TTGCTTTGTTCAT |
| | | | | TGAAGGTGATTTTTTAGTTTTAAAATATATATATCTCATAATATACAATGTTGAT |
| | | | | GGTATATCAATC |
| | | | | CATCAAGCATTTTATTAGAAAGACTTTATAATATTATTAATTTTTCTTTAGTTTTC |
| | | | | AGTAGATAAAGT |
| | | | | AGTTTCAGTAGATAAAGACTTTATAATATTATTAATTTTTCTTTTAGTTTTCAGT |
| | | | | CTCATCATGGGA |
| | | | | GTTAGAAAATACCAGCATCACTTAATTGTAAAGAGAAAATGTGAAAGTATTTT |
| | | | | CCAATCCAATGTT |
| | | | | TCAAGTGCCTCACTTATTGTGAATCTCAAATATCATCTTGAGATGGTTTAATGG |
| | | | | CAGTTTGGCCAA |
| | | | | GTGATTTCTAAGAGATTTTATTAATTTATGCATTAACAATAGTATTCAACTTTCT |
| | | | | TAGGCATTATAA |
| | | | | CCTAACCCGATCATTTGATTGGAATAACTGAATCATTCATTGATTGGAATAAAA |
| | | | | TAAAGAAAACAA |
| | | | | AATGCATCTCACATGCCAGAAGACCTTTACCAGAAGTGGTTCATGAAAATCAG |
| | | | | TATGCATTGACC |
| | | | | ATTCTCTGCCTTTCCCTTTTGGAAAATTTTTTTGTACCTTCATATCACAGCTGAA |
| | | | | TAGTTATCAATGC |
| | | | | CATTTCCATTTGGAAGTATCTAAATAACCACAATGTGATCACAGCTTCTCCACG |
| | | | | AGCCTTCCTGAA |
| | | | | GTAAAGCTGGTATGCTTGCTATAATACTTGGAACCTGTTGCTTTCTCTTCCTCA |
| | | | | CTCTTTAAATGTC |
| | | | | ATAAGGTCACACAGTTCTTGGAATCAGAACTATTCAGTTATTTCTTCATGATTC |
| | | | | TGCCTGGCTTTCT |
| | | | | GAGTCTTCTCCTTCGGCATGTTATGTAAAAGTCTGAACAACAATGATGACGCA |
| | | | | GACGAGAGCAAC |
| | | | | ATACGTTTTTATTTTTTGATAGGTTTTTATTTTTTTGATACCAGAGGGAATTAAC |
| | | | | TATGATCACATC |
| | | | | GTGCTCTCTCTGTTCAGCATCACACGCCCACATACTGCAGTGTGGGAGTAGAT |
| | | | | TGGTACTTACTGA |
| | | | | GCACTTAGAAACATCACGCATCAGGAACCGGGTCAGGGTGACAGCATCCTAC |
| | | | | GGTCATATCATG |
| 57121 | LPAR5 | HSX2_01910604 | 394 | AGCAACACGGAGCACAGGTCTCTGCTGCTGATGA |
| 4038 | LRP4 | HSX2_01040417 | 395 | GGCCAGAGCCTGTGCTGCTGTTCGCCAATCGCATCGACATCCGGCAGGTGCTG |
| | | | | CCACACCGCTCT |
| | | | | GAGTACACACTGCTGCTTAACAACCTGGAGAATGCCATTGCCCTTGATTTCCAC |
| | | | | CACCGCCGCGA |
| | | | | GCTTGTCTTCTGGTCAGATGTCACCCTGGACCGGATCCTCCGTGCCAACCTCAA |
| | | | | CGGCAGCAACGTGGAGGAGGTTGTGTCTACTGGGCTGGAGAGCCCAG |
| 4038 | LRP4 | HSX2_01040427 | 396 | TACCATTTACTGGACAGACTGGGGCAACACCCCCCGTATTGAGGCCTCCAGCA |
| | | | | TGGATGGCTCTG |
| | | | | GACGCCGCATCATTGCCGATACCCATCTCTTCTGGCCCAATGGCCTCACCATCG |
| | | | | ACTATGCCGGG |
| | | | | CGCCGTATGTACTGGGTGGATGCTAAGCACCATGTCATCGAGAGGGCCAATC |
| | | | | TGGATGGGAGTC ACCGTAAGGCTGTCATTAGCCAGG |
| 4038 | LRP4 | HSX2_01040469 | 397 | TGTCTACACCATGTGCTATGGAGAATGGCGGCTGTAGCCACCTGTGTCTTAGG |
| | | | | TCCCCAAATCCA |
| | | | | AGCGGATTCAGCTGTACCTGCCCCACAGGCATCAACCTGCTGTCTGATGGCAA |
| | | | | GACCTGCTCACC AG |
| 4038 | LRP4 | HSX2_01040499 | 398 | GTTTTAACAAGTGCGGCTCGAGAAATGGCGGCTGCTCCCACCTCTGCTTGCCT |
| | | | | CGGCCTTCTGGC |
| | | | | TTCTCCTGTGCCTGCCCCACTGGCATCCAGCTGAAGGGAGATGGGAAGACCTG |
| | | | | TGATCCCTCTCC |
| | | | | TGAGACCTACCTGCTCTTCTCCAGCCGTGGCTCCATCCGGCGTATCTCACTGGA |
| | | | | CACCAGTGACCA |
| | | | | CACCGATGTGCATGTCCCTGTTCCTGAGCTCAACAATGTCATCTCCCTGGACTA |
| | | | | TGACAGCGTGG |
| | | | | ATGGAAAGGTCTATTACACAGATGTGTTCCTGGATGTTATCAG |
| 4038 | LRP4 | HSX2_01040509 | 399 | GTACCTCTTCTGGACAGACTGGGGCCACATTGCCAAGATCGAACGGGCAAAC |
| | | | | TTGGATGGTTCTG |
| | | | | AGCGGAAGGTCCTCATCAACACAGACCTGGGTTGGCCCAATGGCCTTACCCTG |
| | | | | GACTATGATACC CGCAG |
| 4038 | LRP4 | HSX2_01040514 | 400 | GATCTACTGGGTGGATGCGCATCTGGACCGGATCGAGAGTGCTGACCTCAAT |
| | | | | GGGAAACTGCGG |
| | | | | CAGGTCTTGGTCAGCCATGTGTCCCACCCCTTTGCCCTCACACAG |
| 4038 | LRP4 | HSX2_01040519 | 401 | CAAGACAGGTGGATCTACTGGACAGACTGGCAGACCAAGTCAATCCAGCGTG |
| | | | | TTGACAAATACT |
| | | | | CAGGCCGGAACAAGGAGACAGTGCTGGCAAATGTGGAAGGACTCATGGATA |
| | | | | TCATCGTGGTTTC CCCTCAGCGGCAGACAG |
| 4038 | LRP4 | HSX2_01040529 | 402 | TGCCTGGCCTGGTACCACCAGCTCCTAGGGCTACTGGCATGAGTGAAAAGAG |
| | | | | CCCAGTGCTACC |
| | | | | CAACACACCACCTACCACCTTGTATTCTTCAACCACCCGGACCCGCACGTCTCT |
| | | | | GGAGGAGGTGG AAGGAAG |
| 4038 | LRP4 | HSX2_01040534 | 403 | ATGCTCTGAAAGGGATGCCAGGCTGGGCCTCTGTGCACGTTCCAATGACGCT |
| | | | | GTTCCTGCTGCTC CAG |
| 4038 | LRP4 | HSX2_01040539 | 404 | GGGAAGGACTTCATATCAGCTACGCCATTGGTGGACTCCTCAGTATTCTGCTG |
| | | | | ATTTTGGTGGTGATTGCAGCTTTGATGCTGTACAG |
| 4038 | LRP4 | HSX2_01040549 | 405 | CTGGGGATGATGCTGAGTGGGATGACCTCAAGCAACTGCGAAGCTCACGGG |
| | | | | GGGGCCTCCTCCG |
| | | | | GGATCATGTATGCATGAAGACAGACACGGTGTCCATCCAGGCCAGCTCTGGC |
| | | | | TCCCTGGATGAC |
| | | | | ACAGAGACGGAGCAGCTGTTACAGGAAGAGCAGTCTGAGTGTAGCAGCGTC |
| | | | | CATACTGCAGCCA |
| | | | | CTCCAGAAAGACGAGGCTCTCTGCCAGACACGGGCTGGAAACATGAACGCAA |
| | | | | GCTCTCCTCAGA |
| | | | | GAGCCAGGTCTAAATGCCCACATTCTCTTCCCTGCCTGCCTGTTCCTTCTCCTTT |
| | | | | ATGGACGTCTAG |
| | | | | TCCTTGTGCTCGCTTACACCGCAGGCCCCGCTTCTGTGTGCTTGTCCTCCTCCTC |
| | | | | CTCCCACCCCAT |
| | | | | AACTGTTCCTAAGCCTTCACCGGAGCTGTTTACCACGTGAGTCCATAACTACCT |
| | | | | GTGCACAAGAAA |
| | | | | TGATGGCACATCACGAGAATTTAGACCTGGATTTTACCATGAACCTCACATCTT |
| | | | | GTACTCCATCCT |
| | | | | GGGCCCCCTGAAACTGCTTATTCGTGATTCCTCACCAGCGTAGAGCTCCACCTC |
| | | | | CCCTTTCCCCAG |
| | | | | TACCCTCAGTGCCTGCTTCTCAGTGCTGATGCAGCTGATGACCCAGGACTGCG |
| | | | | CTCTGCCCCATCA |
| | | | | CAGCCAGCATGACTGCTTCTCTGAGAGAACTTGCCCATCAGGGGCTGGGACAT |
| | | | | GGGGGTGTGGG |
| | | | | TAAAGACAGGGATGAAGGATAGAGGCTGAGAGAAGAAGGAAGAATCAGCCC |
| | | | | AGCAGGTATGG |
| | | | | GCATCTGGGAAACCTCCAGCCTCAAGTGTGTTAGTAACATGAAAAAGCTTTGG |
| | | | | GGGGTAGTTGG |
| | | | | ATCTGGGTGTCTGGTCCATTGCTGGCAGTGGACATTATTCTTGCCCTAAGAGA |
| | | | | CACTGCCTTTTCA |
| | | | | GCAGCAGATACTGGTGAGATGGGGGTGGCTCAGGCTGTTCTTCCTCCTCCTAG |
| | | | | AATGTCTGGAGC |
| | | | | TGTTTCTACATTCAGATAACTGGGTCCCCTATCACAAGGCTACTGGCTAATAGG |
| | | | | AATTCCCTCCTG |
| | | | | GTGCCACCACTGGCCAGTACCTTTCCTAAGTCTTTGCTCAAATTAACCAGGTTG |
| | | | | TGAGCCAGTGGC |
| | | | | TTGAGTGAATGTTAGGCCTTGGGGGCTGAGTCTCTGAAAAGTCTAAGAAGCT |
| | | | | CTGCCTAGACCAA |
| | | | | ATATGGTATACCTCCTGACCCCTCTCTCCCTCATGTCCTGGGATTCTGGGGAAG |
| | | | | AGACCTAGAAAC |
| | | | | AAGCTTTCAAAGAAAAACCAGAAGTTGTCATAAATGGTCAGAAAGAACGATC |
| | | | | AGGTTGGAGACT |
| | | | | TGGGAAACCCAGGGCCTAAAGAGAAGTATCCATGAGGGTCAAACTTCCTGTT |
| | | | | GAACTTCCTATGT |
| | | | | TCTTTCTCAAGTGCTCAGGGATCTAAGTTAGTGGACAGCAAGCCTGTGGCTAC |
| | | | | GGGGTGGTGATG |
| | | | | TTCCTCTTCCAGCTGTCCCCTCAGCTAAGGGGCTTAGTTTCCATGTGGGATGCC |
| | | | | ATCACTTGGTTC |
| | | | | ATGCTCATTCACACAAAGGGCACGTGTCTCAGCCTGGTATCAGGGAAATTGAG |
| | | | | ACTTATTTTTGCC |
| | | | | CTAAAACGTCTCCCTAGCTGTTCTTCGTGGGGTTTTTTTGTTTGTTTTTTTGCCT |
| | | | | AATTTGCTTTTTC |
| | | | | TGACCAAGCCTTGTGGCACCAGCAATCTCCAAAGTCCTGTGGTGGGAGGGCT |
| | | | | GAATAAATAAAA |
| | | | | ATACAAAGAGGTGGGTAAGGAGTAGGAAGGTAGAGAGCACCACTGATGAGG |
| | | | | CCCTCCTAGCCC |
| | | | | ATGGCAGACCCAGACCTCTTCTCCCCCAGGAATTAGAAGTGGCAGGAGAGAA |
| | | | | CAACAGGGGCTG |
| | | | | GGAATGGAGGGGAGAATTTCTAGGGGAAGTTTCCTGAGTTGAAACTTCTCCT |
| | | | | GTGGTTACTGGTA |
| | | | | TTGAGAAATCAGCTACCAAAGTGAAAAAGGACAAGATCAATTCTTTTCTAGTC |
| | | | | AGTTCTAAGACT |
| | | | | GCTAGAGAGAGATACCAGGCCCTTAGCCTTGCTCTCAGTAGCGTCAGCCCCAG |
| | | | | TTCTGAGCCTCC |
| | | | | CCACATTACACTTAACAAGCAGTAAAGGAGTGAGCACTTTGGGTCCTTAGACT |
| | | | | CACGTCTGGGGA |
| | | | | GGAAGAGCAAGTAGAAAAGTGGCATTTTCTTGATTGGAAAGGGGGAAGGAT |
| | | | | CTTATTGCACTTG |
| | | | | GGCTGTTCAGAATGTAGAAAGGACATATTTGAGGAAGTATCTATTTGAGCACT |
| | | | | GATTTACTCTGTA |
| 4038 | LRP4 | HSX2_01040550 | 406 | TGTAATGGTTTTAACGTTACTCACTGGAGAGATTGGACTTTCTGGAGTTATTTA |
| | | | | ACCACTATGTTC |
| | | | | AGTATTTTAGGACTTTATGATAATTTAATATAAATTTAGCTTTTCTTAATCA |
| 11216 | AKAP10 | HSX2_01559349 | 407 | ATCTTGTCTGGACTACCAGACTCAAGAGACCAAATCAAGCCTTTCTAAGACCCT |
| | | | | TGAACAAGTCTT |
| | | | | GCACGACACTATTGTCCTCCCTTACTTCATTCAATTCATGGAACTTCGGCGAAT |
| | | | | GGAGCATTTGGT |
| | | | | GAAATTTTGGTTAGAGGCTGAAAGTTTTCATTCAACAACTTGGTCGCGAATAA |
| | | | | GAGCACACAGTC |
| | | | | TAAACACAGTGAAGCAGAGCTCACTGGCTGAGCCTGTCTCTCCATCTAAAAAG |
| | | | | CATGAAACTACA |
| | | | | GCGTCTTTTTTAACTGATTCTCTTGATAAGAGATTGGAGGATTCTGGCTCAGCA |
| | | | | CAGTTGTTTATG |
| | | | | ACTCATTCAGAAGGAATTGACCTGAATAATAGAACTAACAGCACTCAGAATCA |
| | | | | CTTGCTGCTTTCC |
| | | | | CAGGAATGTGACAGTGCCCATTCTCTCCGTCTTGAAATGGCCAGAGCAGGAAC |
| | | | | TCACCAAGTTTC |
| | | | | CATGGAAACCCAAGAATCTTCCTCTACACTTACAGTAGCCAGTAGAAATAGTC |
| | | | | CCGCTTCTCCACT AAAAGAATTGTCAGGAAAACTAATGAAAA |
| 115353 | LRRC42 | HSX2_02172282 | 408 | TGTAACTCAGCTCCACCTGAAGGATAATTGTTTATCTGATGCTGGGGTGCGGA |
| | | | | AGATGACAGCAC |
| | | | | CAGTTCGAGTGATGAAAAGAGGCCTTGAGAATCTAACATTATTAGACTTATCA |
| | | | | T |
| 120892 | LRRK2 | HSX2_02185024 | 409 | TTCCCTGAGCAGCGGACGTTCATGCTGGGAGGGCGGCGGGTTGGAAGCAGG |
| | | | | TGCCACCATGGC |
| | | | | TAGTGGCAGCTGTCAGGGGTGCGAAGAGGACGAGGAAACTCTGAAGAAGTT |
| | | | | GATAGTCAGGCT |
| | | | | GAACAATGTCCAGGAAGGAAAACAGATAGAAACGCTGGTCCAAATCCTGGAG |
| | | | | GATCTGCTGGTG TTCACGTACTCCGAGCGCG |
| 120892 | LRRK2 | HSX2_02185048 | 410 | GTGGGTTGGTCACTTCTGTGCAAATTAATAGAAGTCTGTCCAGGTACAATGCA |
| | | | | AAGCTTAATGGG |
| | | | | ACCCCAGGATGTTGGAAATGATTGGGAAGTCCTTGGTGTTCACCA |
| 120892 | LRRK2 | HSX2_02185064 | 411 | ATTGATTCTTAAAATGCTAACAGTTCATAATGCCAGTGTAAACTTGTCAGTGAT |
| | | | | TGGACTGAAGAC CTTAGATCTCCTCCTAACTTCAG |
| 120892 | LRRK2 | HSX2_02185088 | 412 | TCTCAGAGGAGCAACTGACTGAATTTGTTGAGAACAAAGATTATATGATATTG |
| | | | | TTAAGTGCGTTA |
| | | | | ACAAATTTTAAAGATGAAGAGGAAATTGTGCTTCATGTGCTGCATTGTTTACA |
| | | | | TTCCCTAGCGATT CCTT |
| 120892 | LRRK2 | HSX2_02185098 | 413 | GCAATAATGTGGAAGTCCTCATGAGTGGCAATGTCAGGTGTTATAATATTGTG |
| | | | | GTGGAAGCTATG |
| | | | | AAAGCATTCCCTATGAGTGAAAGAATTCAAGAAGTGAGTTGCTGTTTGCTCCA |
| | | | | TAGGCTTACATTA G |
| 120892 | LRRK2 | HSX2_02185108 | 414 | GTAATTTTTTCAATATCCTGGTATTAAACGAAGTCCATGAGTTTGTGGTGAAAG |
| | | | | CTGTGCAGCAGT |
| | | | | ACCCAGAGAATGCAGCATTGCAGATCTCAGCGCTCAGCTGTTTGGCCCTCCTC |
| | | | | A |
| 120892 | LRRK2 | HSX2_02185118 | 415 | CTGAGACTATTTTCTTAAATCAAGATTTAGAGGAAAAGAATGAGAATCAAGAG |
| | | | | AATGATGATGAG |
| | | | | GGGGAAGAAGATAAATTGTTTTGGCTGGAAGCCTGTTACAAAGCATTAACGT |
| | | | | GGCATAGAAAGA ACAAGCACGTGCAG |
| 120892 | LRRK2 | HSX2_02185128 | 416 | GAGGCCGCATGCTGGGCACTAAATAATCTCCTTATGTACCAAAACAGTTTACA |
| | | | | TGAGAAGATTGG AGATGAAGATGGCCA |
| 120892 | LRRK2 | HSX2_02185137 | 417 | TTTCCCAGCTCATAGGGAAGTGATGCTCTCCATGCTGATGCATTCTTCATCAAA |
| | | | | G |
| 120892 | LRRK2 | HSX2_02185139 | 418 | GAAGTTTTCCAGGCATCTGCGAATGCATTGTCAACTCTCTTAGAACAAAATG |
| 120892 | LRRK2 | HSX2_02185153 | 419 | TTAATTTCAGAAAAATACTGTTATCAAAAGGAATACACCTGAATGTTTTGGAGT |
| | | | | TAATGCAGAAGC |
| | | | | ATATACATTCTCCTGAAGTGGCTGAAAGTGGCTGTAAAATGCTAAATCATCTTT |
| | | | | TTGAAGGAAG |
| 120892 | LRRK2 | HSX2_02185176 | 420 | GCATGCCAGAAGAATCCAGGGAGGATACAGAATTTCATCATAAGCTAAATATG |
| | | | | GTTAAAAAACA |
| | | | | GTGTTTCAAGAATGATATTCACAAACTGGTCCTAGCAGCTTTGAACAGG |
| 120892 | LRRK2 | HSX2_02185188 | 421 | TTCATTGGAAATCCTGGGATTCAGAAATGTGGATTAAAAGTAATTTCTTCTATT |
| | | | | GTACATTTTCCTG |
| | | | | ATGCATTAGAGATGTTATCCCTGGAAGGTGCTATGGATTCAGTGCTTCACACA |
| | | | | CTGCAGATGTAT CCAGATGACCAAG |
| 120892 | LRRK2 | HSX2_02185200 | 422 | AAATTCAGTGTCTGGGTTTAAGTCTTATAGGATACTTGATTACAAAGAAGAAT |
| | | | | GTGTTCATAGGAA |
| | | | | CTGGACATCTGCTGGCAAAAATTCTGGTTTCCAGCTTATACCGATTTAAGGATG |
| | | | | TTGCTGAAATAC AGACTAAA |
| 120892 | LRRK2 | HSX2_02185226 | 423 | TTTCTAAACCTCTGTTGCAAGTGTTTTGCAAAAGTAGCTATGGATGATTACTTA |
| | | | | AAAAATGTGATG |
| | | | | CTAGAGAGAGCGTGTGATCAGAATAACAGCATCATGGTTGAATGCTTGCTTCT |
| | | | | ATTGGGAGCAGA TGCCAATCAAGCAAAGGAGGGATCTTCTTTAATTTGTCAG |
| 120892 | LRRK2 | HSX2_02185236 | 424 | GTATGTGAGAAAGAGAGCAGTCCCAAATTGGTGGAACTCTTACTGAATAGTG |
| | | | | GATCTCGTGAACA |
| | | | | AGATGTACGAAAAGCGTTGACGATAAGCATTGGGAAAGGTGACAGCCAGATC |
| | | | | ATCAGCTTGCTC |
| | | | | TTAAGGAGGCTGGCCCTGGATGTGGCCAACAATAGCATTTGCCTTGGAGGATT |
| | | | | TTGTATAGGAAA |
| | | | | AGTTGAACCTTCTTGGCTTGGTCCTTTATTTCCAGATAAGACTTCTAATTTAAGG |
| | | | | AAACAAACAA |
| 120892 | LRRK2 | HSX2_02185241 | 425 | ATATAGCATCTACACTAGCAAGAATGGTGATCAGATATCAGATGAAAAGTGCT |
| | | | | GTGGAAGAAGG |
| | | | | AACAGCCTCAGGCAGCGATGGAAATTTTTCTGAAGATGTGCTGTCTAAATTTG |
| | | | | ATGAATGGACCT |
| | | | | TTATTCCTGACTCTTCTATGGACAGTGTGTTTGCTCAAAGTGATGACCTGGATA |
| | | | | GTGAAG |
| 120892 | LRRK2 | HSX2_02185269 | 426 | GGTCATCAAAACTTCAATCCCATATGAGGCATTCAGACAGCATTTCTTCTCTGG |
| | | | | CTTCTGAGAGAG |
| | | | | AATATATTACATCACTAGACCTTTCAGCAAATGAACTAAGAGATATTGATGCCC |
| | | | | TAAGCCAGAAAT |
| | | | | GCTGTATAAGTGTTCATTTGGAGCATCTTGAAAAGCTGGAGCTTCACCAGAAT |
| | | | | GCACTCACGAGC TTTCCACAACAGCTATGTGAA |
| 120892 | LRRK2 | HSX2_02185278 | 427 | ACTCTGAAGAGTTTGACACATTTGGACTTGCACAGTAATAAATTTACATCATTT |
| | | | | CCTTCTTATTTGT |
| | | | | TGAAAATGAGTTGTATTGCTAATCTTGATGTCTCTCGAAATGACATTGGACCCT |
| | | | | CAGTGGTTTTAG |
| | | | | ATCCTACAGTGAAATGTCCAACTCTGAAACAGTTTAACCTGTCATATAACCAGC |
| | | | | TGTCTTTTGTACC |
| | | | | TGAGAACCTCACTGATGTGGTAGAGAAACTGGAGCAGCTCATTTTAGAAGG |
| 120892 | LRRK2 | HSX2_02185287 | 428 | AAATAAAATATCAGGGATATGCTCCCCCTTGAGACTGAAGGAACTGAAGATTT |
| | | | | TAAACCTTAGTA |
| | | | | AGAACCACATTTCATCCCTATCAGAGAACTTTCTTGAGGCTT GTCCTAAAGTGG |
| | | | | AGAGTTTCAGTG CCAGAATGAATTTTCTTG |
| 120892 | LRRK2 | HSX2_02185299 | 429 | CTGCTATGCCTTTCTTGCCTCCTTCTATGACAATCCTAAAATTATCTCAGAACAA |
| | | | | ATTTTCCTGTATT CCAGAAGCAATTTTAAATCTTCCACA |
| 120892 | LRRK2 | HSX2_02185363 | 430 | GTCGTGAGGAATTCTATAGTACTCATCCCCATTTTATGACGCAGCGAGCATTGT |
| | | | | ACCTTGCTGTCT |
| | | | | ATGACCTCAGCAAGGGACAGGCTGAAGTTGATGCCATGAAGCCTTGGCTCTTC |
| | | | | AATATAAAG |
| 120892 | LRRK2 | HSX2_02185381 | 431 | GCTCGCGCTTCTTCTTCCCCTGTGATTCTCGTTGGCACACATTTGGATGTTTCTG |
| | | | | ATGAGAAGCAA |
| | | | | CGCAAAGCCTGCATGAGTAAAATCACCAAGGAACTCCTGAATAAGCGAGGGT |
| | | | | TCCCTGCCATAC |
| | | | | GAGATTACCACTTTGTGAATGCCACCGAGGAATCTGATGCTTTGGCAAAACTT |
| | | | | CGGAAAACCATC ATAAACGAGAGCCTTAATTTCAAG |
| 120892 | LRRK2 | HSX2_02185395 | 432 | ATCCGAGATCAGCTTGTTGTTGGACAGCTGATTCCAGACTGCTATGTAGAACTT |
| | | | | GAAAAAATCATT |
| | | | | TTATCGGAGCGTAAAAATGTGCCAATTGAATTTCCCGTAATTGACCGGAAACG |
| | | | | ATTATTACAACTA |
| | | | | GTGAGAGAAAATCAGCTGCAGTTAGATGAAAATGAGCTTCCTCACGCAGTTCA |
| | | | | CTTTCTAAATGA ATCAG |
| 120892 | LRRK2 | HSX2_02185411 | 433 | GAGTCCTTCTTCATTTTCAAGACCCAGCACTGCAGTTAAGTGACTTGTACTTTG |
| | | | | TGGAACCCAAGT GGCTTTGTAAAATCATGGCACAG |
| 120892 | LRRK2 | HSX2_02185433 | 434 | TTTGTCTGACCACAGGCCTGTGATAGAGCTTCCCCATTGTGAGAACTCTGAAAT |
| | | | | TATCATCCGACT |
| | | | | ATATGAAATGCCTTATTTTCCAATGGGATTTTGGTCAAGATTAATCAATCGATT |
| | | | | ACTTGAGATTTCA CCTTACATGCTTTCAGGGAGAG |
| 120892 | LRRK2 | HSX2_02185443 | 435 | AACGAGCACTTCGCCCAAACAGAATGTATTGGCGACAAGGCATTTACTTAAAT |
| | | | | TGGTCTCCTGAA |
| | | | | GCTTATTGTCTGGTAGGATCTGAAGTCTTAGACAATCATCCAGAGAGTTTCTTA |
| | | | | AAAATTACAGTT CCTTCTTGTAGAAAAG |
| 120892 | LRRK2 | HSX2_02185455 | 436 | GCTGTATTCTTTTGGGCCAAGTTGTGGACCACATTGATTCTCTCATGGAAGAAT |
| | | | | GGTTTCCTGGGT |
| | | | | TGCTGGAGATTGATATTTGTGGTGAAGGAGAAACTCTGTTGAAGAAATGGGC |
| | | | | ATTATATAGTTTT |
| | | | | AATGATGGTGAAGAACATCAAAAAATCTTACTTGATGACTTGATGAAGAAAGC |
| | | | | AGAGGAAG |
| 120892 | LRRK2 | HSX2_02185470 | 437 | GAGATCTCTTAGTAAATCCAGATCAACCAAGGCTCACCATTCCAATATCTCAGA |
| | | | | TTGCCCCTGACT |
| | | | | TGATTTTGGCTGACCTGCCTAGAAATATTATGTTGAATAATGATGAGTTGGAAT |
| | | | | TTGAACAAGCTC CAGAGTTTCTCCTAG |
| 120892 | LRRK2 | HSX2_02185480 | 438 | GTGATGGCAGTTTTGGATCAGTTTACCGAGCAGCCTATGAAGGAGAAGAAGT |
| | | | | GGCTGTGAAGAT TTTTAATAAACATACATCACTCAGGCTGTTAAGACAA |
| 120892 | LRRK2 | HSX2_02185490 | 439 | GAGCTTGTGGTGCTTTGCCACCTCCACCACCCCAGTTTGATATCTTTGCTGGCA |
| | | | | GCTGGGATTCGT |
| | | | | CCCCGGATGTTGGTGATGGAGTTAGCCTCCAAGGGTTCCTTGGATCGCCTGCT |
| | | | | TCAGCAGGACAA |
| | | | | AGCCAGCCTCACTAGAACCCTACAGCACAGGATTGCACTCCACGTAGCTGATG |
| | | | | GTTTGAG |
| 120892 | LRRK2 | HSX2_02185500 | 440 | ATACCTCCACTCAGCCATGATTATATACCGAGACCTGAAACCCCACAATGTGCT |
| | | | | GCTTTTCACACT |
| | | | | GTATCCCAATGCTGCCATCATTGCAAAGATTGCTGACTACGGCATTGCTCAGTA |
| | | | | CTGCTGTAGAAT GGGGATAAAAACATCAGAGGGCACACCAG |
| 120892 | LRRK2 | HSX2_02185515 | 441 | GGTTTCGTGCACCTGAAGTTGCCAGAGGAAATGTCATTTATAACCAACAGGCT |
| | | | | GATGTTTATTCAT |
| | | | | TTGGTTTACTACTCTATGACATTTTGACAACTGGAGGTAGAATAGTAGAGGGT |
| | | | | TTGAAGTTTCCAA ATGAGTTTGATGAATTAGAAATACAAGGAAAATTACCTG |
| 120892 | LRRK2 | HSX2_02185530 | 442 | ATCCAGTTAAAGAATATGGTTGTGCCCCATGGCCTATGGTTGAGAAATTAATT |
| | | | | AAACAGTGTTTGA AAGAAAATCCTCAAGAAAGGCCTACTTCTGCCCAG |
| 120892 | LRRK2 | HSX2_02185541 | 443 | GTCTTTGACATTTTGAATTCAGCTGAATTAGTCTGTCTGACGAGACGCATTTTA |
| | | | | TTACCTAAAAAC |
| | | | | GTAATTGTTGAATGCATGGTTGCTACACATCACAACAGCAGGAATGCAAGCAT |
| | | | | TTGGCTGGGCTG |
| | | | | TGGGCACACCGACAGAGGACAGCTCTCATTTCTTGACTTAAATACTGAAGGAT |
| | | | | ACACTTCTGAG |
| 120892 | LRRK2 | HSX2_02185551 | 444 | GAAGTTGCTGATAGTAGAATATTGTGCTTAGCCTTGGTGCATCTTCCTGTTGAA |
| | | | | AAGGAAAGCTG |
| | | | | GATTGTGTCTGGGACACAGTCTGGTACTCTCCTGGTCATCAATACCGAAGATG |
| | | | | GGAAAAAGAGAC |
| | | | | ATACCCTAGAAAAGATGACTGATTCTGTCACTTGTTTGTATTGCAATTCCTTTTC |
| | | | | CAAGCAAAG |
| 120892 | LRRK2 | HSX2_02185560 | 445 | CAAACAAAAAAATTTTCTTTTGGTTGGAACCGCTGATGGCAAGTTAGCAATTTT |
| | | | | TGAAGATAAGAC TGTTAAG |
| 120892 | LRRK2 | HSX2_02185571 | 446 | CTTAAAGGAGCTGCTCCTTTGAAGATACTAAATATAGGAAATGTCAGTACTCC |
| | | | | ATTGATGTGTTTG |
| | | | | AGTGAATCCACAAATTCAACGGAAAGAAATGTAATGTGGGGAGGATGTGGCA |
| | | | | CAAAGATTTTCT |
| | | | | CCTTTTCTAATGATTTCACCATTCAGAAACTCATTGAGACAAGAACAAGCCAAC |
| | | | | T |
| 120892 | LRRK2 | HSX2_02185581 | 447 | GTTTTCTTATGCAGCTTTCAGTGATTCCAACATCATAACAGTGGTGGTAGACAC |
| | | | | TGCTCTCTATATT |
| | | | | GCTAAGCAAAATAGCCCTGTTGTGGAAGTGTGGGATAAGAAAACTGAAAAAC |
| | | | | TCTGTGGACTAA TAGACTGCGTGCACTTTTTAAG |
| 120892 | LRRK2 | HSX2_02185595 | 448 | GGAGGTAATGGTAAAAGAAAACAAGGAATCAAAACACAAAATGTCTTATTCT |
| | | | | GGGAGAGTGAAA |
| | | | | ACCCTCTGCCTTCAGAAGAACACTGCTCTTTGGATAGGAACTGGAGGAGGCCA |
| | | | | TATTTTACTCCT |
| | | | | GGATCTTTCAACTCGTCGACTTATACGTGTAATTTACAACTTTTGTAATTCGGTC |
| | | | | AGAGTCATGAT GACAGCACAGCTAG |
| 120892 | LRRK2 | HSX2_02185609 | 449 | GAAGCCTTAAAAATGTCATGCTGGTATTGGGCTACAACCGGAAAAATACTGAA |
| | | | | GGTACACAAAA GCAGAAAG |
| 120892 | LRRK2 | HSX2_02185619 | 450 | AGATACAATCTTGCTTGACCGTTTGGGACATCAATCTTCCACATGAAGTGCAAA |
| | | | | ATTTAGAAAAAC |
| | | | | ACATTGAAGTGAGAAAAGAATTAGCTGAAAAAATGAGACGAACATCTGTTGA |
| | | | | GTAAGAGAGAAA |
| | | | | TAGGAATTGTCTTTGGATAGGAAAATTATTCTCTCCTCTTGTAAATATTTATTTT |
| | | | | AAAAATGTTCAC ATGGAAAGGGTACTCACA |
| 120892 | LRRK2 | HSX2_02185621 | 451 | TTTTTTGAAATAGCTCGTGTGTATGAAGGAATGTTATTATTTTTAATTTAAATAT |
| | | | | ATGTAAAAATAC |
| | | | | TTACCAGTAAATGTGTATTTTAAAGAACTATTTAAAACACAATGTTATATTTCTT |
| | | | | ATAAATACCAGT |
| | | | | TACTTTCGTTCATTAATTAATGAAAATAAATCTGTGAAGTACCTAATTTAAGTA |
| | | | | CTCATACTAAAAT |
| | | | | TTATAAGGCCGATAATTTTTTGTTTTCTTGTCTGTAATGGAGGTAAACTTTATTT |
| | | | | TAAATTCTGTGCT |
| | | | | TAAGACAGGACTATTGCTTGTCGATTTTTCTAGAAATCTGCACGGTATAATGAA |
| | | | | AATATTAAGACA |
| | | | | GTTTCCCATGTAATGTATTCCTTCTTAGATTGCATCGAAATGCACTATCATATAT |
| | | | | GCTTGTAAATAT |
| | | | | TCAAATGAATTTGCACTAATAAAGTCCTTTGTTGGTATGTGAATTCTCTTTGTTG |
| | | | | CTGTTGCAAACA |
| | | | | GTGCATCTTACACAACTTCACTCAATTCAAAAGAAAACTCCATTAAAAGTACTA |
| | | | | ATGAAAAAACAT |
| | | | | GACATACTGTCAAAGTCCTCATATCTAGGAAAGACACAGAAACTCTCTTTGTCA |
| | | | | CAGAAACTCTCT |
| | | | | GTGTCTTTCCTAGACATAATAGAGTTGTTTTTCAACTCTATGTTTGAATGTGGA |
| | | | | TACCCTGAATTTT |
| | | | | GTATAATTAGTGTAAATACAGTGTTCAGTCCTTCAAGTGATATTTTTATTTTTTT |
| | | | | ATTCATACCACTA |
| | | | | GCTACTTGTTTTCTAATCTGCTTCATTCTAATGCTTATATTCATCTTTTCCCTAAA |
| | | | | TTTGTGATGCTG |
| | | | | CAGATCCTACATCATTCAGATAGAAACCTTTTTTTTTTTCAGAATTATAGAATTC |
| | | | | CACAGCTCCTAC |
| | | | | CAAGACCATGAGGATAAATATCTAACACTTTTCAGTTGCTGAAGGAGAAAGGA |
| | | | | GCTTTAGTTATG |
| | | | | ATGGATAAAAATATCTGCCACCCTAGGCTTCCAAATTATACTTAAATTGTTTAC |
| | | | | ATAGCTTACCACA |
| | | | | ATAGGAGTATCAGGGCCAAATACCTATGTAATAATTTGAGGTCATTTCTGCTTT |
| | | | | AGGAAAAGTACT |
| | | | | TTCGGTAAATTCTTTGGCCCTGACCAGTATTCATTATTTCAGATAATTCCCTGTG |
| | | | | ATAGGACAACTA |
| | | | | GTACATTTAATATTCTCAGAACTTATGGCATTTTACTATGTGAAAACTTTAAATT |
| | | | | TATTTATATTAAG |
| | | | | GGTAATCAAATTCTTAAAGATGAAAGATTTTCTGTATTTTAAAGGAAGCTATGC |
| | | | | TTTAACTTGTTAT |
| | | | | GTAATTAACAAAAAAATCATATATAATAGAGCTCTTTGTTCCAGTGTTATCTCTT |
| | | | | TCATTGTTACTTT |
| | | | | GTATTTGCAATTTTTTTTACCAAAGACAAATTAAAAAAATGAATACCATATTTA |
| | | | | AATGGAATAATAA AGGTTTTTTAAAAACTTTAA |
| 120892 | LRRK2 | HSX2_02576451 | 452 | TTTCCCAGCTCATAGGGAAGTGATGCTCTCCATGCTGATGCATTCTTCATCAAA |
| | | | | GGAAGTTTTCCA GGCATCTGCGAATGCATTGTCAACTCTCTTAGAACAAAATG |
| 4050 | LTB | HSX2_01041650 | 453 | GTCTCAATGGGGGCACTGGGGCTGGAGGGCAGGGGTGGGAGGCTCCAGGG |
| | | | | GAGGGGTTCCCT |
| | | | | CCTGCTAGCTGTGGCAGGAGCCACTTCTCTGGTGACCTTGTTGCTGGCGGTGC |
| | | | | CTATCACTGTCCT |
| | | | | GGCTGTGCTGGCCTTAGTGCCCCAGGATCAGGGAGGACTG |
| 23499 | MACF1 | HSX2_01641182 | 454 | GATGAAAAGGAGCAGCTTATACAGTCCAAGAGTTCCGTTGCCAGTCTCGTTGG |
| | | | | GAGATCAAAAA |
| | | | | CCATCGTTCAGCTAAAACCACGCAGTCCAGACCATGTGTTAAAGAACACCATTT |
| | | | | CTGTCAAGGCT GTCTGTGACTACAGGCAGATCGAG |
| 23499 | MACF1 | HSX2_01641195 | 455 | ATTACTATTTGCAAAAATGATGAATGTGTGCTAGAAGATAATTCTCAGCGGAC |
| | | | | CAAATGGAAAGT |
| | | | | GATCAGCCCCACAGGGAACGAGGCAATGGTGCCGTCAGTCTGCTTCCTCATCC |
| | | | | CCCCACCCAATA AGGATGCCATTGAGATGGCCAGCAG |
| 23499 | MACFI | HSX2_01641210 | 456 | GGTCGAACAATCTTATCAGAAGGTTATGGCCCTTTGGCATCAGCTGCATGTTA |
| | | | | ACACCAAAAGCC |
| | | | | TTATCTCTTGGAACTATCTGCGTAAAGACCTTGACCTTGTACAGACCTGGAACC |
| | | | | TAGAAAAG |
| 9223 | MAGI1 | HSX2_01392893 | 457 | CTATGTCACCTTCACCTGCGTCGGGATTGAGCAAGGGTGAGAGAGAGAGAGA |
| | | | | AATCAATTCCAC GAATTTTGGAGAATGTCCGA |
| 4124 | MAN2A1 | HSX2_01046549 | 458 | ATGTTGGATGTTTACAGTCTAATTTCTTTTGACAATCCAGATGGTGGAGTTTGG |
| | | | | AAGCAAGGATTT |
| | | | | GACATTACTTATGAATCTAATGAATGGGACACTGAACCCCTTCAAGTCTTTGTG |
| | | | | GTGCCTCATTCC CATAACGACCCAG |
| 149175 | MANEAL | HSX2_02487371 | 459 | ATCAGTACAGCATCCAGGTGGCCTTCCACATCCA |
| 4128 | MAOA | HSX2_01047014 | 460 | ATTCCAACTGATGCACCCTGGGAGGCTCAACATGCTGACAAATGGGACAAAA |
| | | | | TGACCATGAAAG AGCTCATTGACAAAATCTGCTGGACAAA |
| 4128 | MAOA | HSX2_01047060 | 461 | GGTGATTCGTCAACCCGTGGGCAGGATTTTCTTTGCGGGCACAGAGACTGCC |
| | | | | ACAAAGTGGAGC |
| | | | | GGCTACATGGAAGGGGCAGTTGAGGCTGGAGAACGAGCAGCTAGGGAG |
| 4128 | MAOA | HSX2_01047065 | 462 | GTCTTAAATGGTCTCGGGAAGGTGACCGAGAAAGATATCTGGGTACAAGAAC |
| | | | | CTGAATCAAAG |
| 4128 | MAOA | HSX2_02507973 | 463 | GAAGAAGAAAATCTGTGAGCTCTATGCCAAAGTGCTGGGATCCCAAGAAGCT |
| | | | | TTACAT |
| 4133 | MAP2 | HSX2_01047357 | 464 | ATTCTTCAGCTTGTCTCTAACCGAGGAAGCATTGATTGGGAGCTACTCATTCA |
| | | | | GAAAATTAAAAG |
| 4133 | MAP2 | HSX2_01047371 | 465 | TTGCAGGAGAAATAACAAGGCATTGAAGAATGGCAGATGAACGGAAAGATG |
| | | | | AAGCAAAGGCAC |
| | | | | CTCACTGGACCTCAGCACCGCTAACAGAGGCATCTGCACACTCACATCCACCT |
| | | | | GAGATTAAGGAT |
| | | | | CAAGGCGGAGCAGGGGAAGGACTTGTCCGAAGCGCCAATGGATTCCCATACA |
| | | | | GGGAGGATGAA |
| | | | | GAGGGTGCCTTTGGAGAGCATGGGTCACAGGGCACCTATTCAAATACCAAAG |
| | | | | AGAATGGGATCA ACGGAGAGCTGACCTCAGCTGACAGAGAAACAGCAG |
| 4133 | MAP2 | HSX2_01047388 | 466 | CAGCAGGTGGGGAATCAGCTCTGGCTCCCAGTGTATTTAAACAGGCAAAGGA |
| | | | | CAAAGTCTCT |
| 4133 | MAP2 | HSX2_01047403 | 467 | GGTCAGAGCCAATTCGCAGAGCAGGGAAGAGTGGTACCTCAACACCCACTAC |
| | | | | CCCTGGGTCTAC TGCCATCACTCCTGGCACCCCACCAAGTTATTCTTCAC |
| 4133 | MAP2 | HSX2_01047404 | 468 | GCACACCAGGCACTCCTGGAACCCCTAGCTATCCCAGGACCCCTCACACACCA |
| | | | | GGAACCCCCAAG |
| | | | | TCTGCCATCTTGGTGCCGAGTGAGAAGAAGGTCGCCATCATACGTACTCCTCC |
| | | | | AAAATCTCCTGC |
| | | | | GACTCCCAAGCAGCTTCGGCTTATTAACCAACCACTGCCAGACCTGAAGAATG |
| | | | | TCAAATCCAAAA |
| | | | | TCGGATCAACAGACAACATCAAATACCAGCCTAAAGGGGGGCAG |
| 4133 | MAP2 | HSX2_01047415 | 469 | GTACAAATTGTTACCAAGAAAATAGACCTAAGCCATGTGACATCCAAATGTGG |
| | | | | CTCTCTGAAGAA CATCCGCCACAGGCCAG |
| 4133 | MAP2 | HSX2_01047421 | 470 | GCGGACGTGTGAAAATTGAGAGTGTAAAACTAGATTTCAAAGAAAAGGCCCA |
| | | | | AGCTAAAGTTGG |
| | | | | TTCTCTTGATAATGCTCATCATGTACCTGGAGGTGGTAATGTCAAG |
| 4133 | MAP2 | HSX2_01047427 | 471 | ATTGACAGCCAAAAGTTGAACTTCAGAGAGCATGCTAAAGCCCGTGTGGACC |
| | | | | ATGGGGCTGAGA |
| | | | | TCATTACACAGTCCCCAGGCAGATCCAGCGTGGCATCACCCCGACGACTCAGC |
| | | | | AATGTCTCCTCG |
| | | | | TCTGGAAGCATCAACCTGCTCGAATCTCCTCAGCTTGCCACTTTGGCTGAGGA |
| | | | | TGTCACTGCTGCA |
| | | | | CTCGCTAAGCAGGGCTTGTGAATATTTCTCATTTAGCATTGAAATAATAATATT |
| | | | | TAGGCATGAGCT CTTGGCAGGAGTGGGCTCTGA |
| 4133 | MAP2 | HSX2_02508006 | 472 | CAGCTGAAGAAACAGCTAATCTGCCTCCTTCTCCACCCCCATCACCTGCCTCAG |
| | | | | AACAGACTGTCA CAGTGGAGGAAG |
| 4133 | MAP2 | HSX2_02508011 | 473 | GGTCAGAGCCAATTCGCAGAGCAGGGAAGAGTGGTACCTCAACACCCACTAC |
| | | | | CCCTGGGTCTAC |
| | | | | TGCCATCACTCCTGGCACCCCACCAAGTTATTCTTCACGCACACCAGGCACTCC |
| | | | | TGGAACCCCTAG |
| | | | | CTATCCCAGGACCCCTCACACACCAGGAACCCCCAAGTCTGCCATCTTGGTGC |
| | | | | CGAGTGAGAAGA |
| | | | | AGGTCGCCATCATACGTACTCCTCCAAAATCTCCTGCGACTCCCAAGCAGCTTC |
| | | | | GGCTTATTAACC |
| | | | | AACCACTGCCAGACCTGAAGAATGTCAAATCCAAAATCGGATCAACAGACAA |
| | | | | CATCAAATACCAG CCTAAAGGGGGGCAG |
| 6441 | SFTPD | HSX2_01215290 | 474 | TTGAGCTCTTCCCAAATGGCCAAAGTGTCGGGGAGAAGATTTTCAAGACAGC |
| | | | | AGGCTTTGTAAAA |
| | | | | CCATTTACGGAGGCACAGCTGCTGTGCACACAGGCTGGTGGACAGTTGGCCT |
| | | | | CTCCACGCTCTGC |
| | | | | CGCTGAGAATGCCGCCTTGCAACAGCTGGTCGTAGCTAAGAACGAGGCTGCT |
| | | | | TTCCTGAGCATG |
| | | | | ACTGATTCCAAGACAGAGGGCAAGTTCACCTACCCCACAGGAGAGTCCCTGG |
| | | | | TCTATTCCAACTG |
| | | | | GGCCCCAGGGGAGCCCAACGATGATGGCGGGTCAGAGGACTGTGTGGAGAT |
| | | | | CTTCACCAATGG |
| | | | | CAAGTGGAATGACAGGGCTTGTGGAGAAAAGCGTCTTGTGGTCTGCGAGTTC |
| | | | | TGAGCCAACTGG |
| | | | | GGTGGGTGGGGCAGTGCTTGGCCCAGGAGTTTGGCCAGAAGTCAAGGCTTA |
| | | | | GACCCTCATGCTG CCAATATCCTAATAAAAAGGTGACCAT |
| 79143 | MBOAT7 | HSX2_02566141 | 475 | CGTGGGAATCATGACAGCCCGACGTGCCCTCCCT |
| 64769 | MEAF6 | HSX2_01981938 | 476 | TTGATCTGAAGTTAAACAAAAAACCACGA |
| 64769 | MEAF6 | HSX2_01981953 | 477 | AATAAAAACCGGCACAGGATTGATCTGAAGTTAA |
| 64769 | MEAF6 | HSX2_02564579 | 478 | GATTGATCTGAAGTTAAACAAAAAACCACGAGCT |
| 9969 | MED13 | HSX2_01458570 | 479 | GAGGAAGTATTCTGCTTCATCAGGTGGTCTATGCGAAGAAGCGACAGCTGCT |
| | | | | AAAGTGGCATCCT |
| | | | | GGGATTTTGTTGAAGCCACACAAAGAACAAATTGCAGTTGTTTGAG |
| 9969 | MED13 | HSX2_01458575 | 480 | GCACAAAAATCTCAAGTCAAGAAATGCTGGACAACAAGGACAGGCACCATCTT |
| | | | | TAGGTCAGCAA |
| | | | | CAACAAATACTTCCTAAGCACAAGACCAATGAGAAGCAAGAAAAGAGTGAAA |
| | | | | AGCCACAGAAAC |
| | | | | GCCCCTTGACTCCTTTTCACCATCGTGTGTCTGTTAGTGATGATGTTGGCATGG |
| | | | | ACGCAGATTCAG |
| | | | | CCAGCCAAAGACTTGTGATCTCTGCTCCAGACAGTCAAGTGAGATTTTCAAAT |
| | | | | ATCCGAACTAATG |
| | | | | ATGTAGCAAAGACTCCTCAGATGCATGGCACCGAAATGGCAAATTCACCTCAA |
| | | | | CCACCCCCACTT |
| | | | | AGTCCTCACCCTTGTGATGTGGTTGATGAAGGAGTGACTAAAACACCTTCAAC |
| | | | | TCCTCAGAGTCA |
| | | | | ACATTTTTATCAAATGCCAACACCAGATCCCTTGGTTCCTTCTAAACCAATGGA |
| | | | | AGATAGGATAGA |
| | | | | CAGTTTGTCCCAGTCTTTCCCACCTCAATATCAGGAAGCTGTAGAACCTACAGT |
| | | | | ATATGTTGGTAC |
| | | | | AGCAGTAAACTTGGAAGAAGATGAAGCCAATATAGCCTGGAAGTATTACAAG |
| | | | | TTCCCAAAGAAA |
| | | | | AAAGATGTAGAGTTTTTACCACCTCAACTTCCAAGTGATAAATTCAAGGATGAT |
| | | | | CCAGTTGGACCT TTTGGACAGGAAAGTGTAACATCAGTTACAGA |
| 9969 | MED13 | HSX2_01458679 | 481 | GCCTCTTTGCACCTCCACGTGCCTTCAGTGCAATCTGACGAGCTGCTTCACAGT |
| | | | | AAACACTCCCAC CCACTTGACTCAAATCAGACTTCAGATGTCCTCAG |
| 9969 | MED13 | HSX2_01458684 | 482 | GTTTGTTTTGGAACAGTACAATGCACTCTCCTGGCTAACCTGTGACCCTGCAAC |
| | | | | CCAGGACAGAC |
| | | | | GCTCATGTCTCCCAATTCATTTTGTGGTGCTGAATCAGTTATATAACTTTATTAT |
| | | | | GAATATGCTGTG |
| | | | | ATCTTCATTTGATGGAACTGTGCAAGAAAAGAACAAGGAAAAATGGATGTTTC |
| | | | | GCTGCAGGAT |
| 4233 | MET | HSX2_01055592 | 483 | ACCTCTCATAATGAAGGCCCCCGCTGTGCTTGCACCTGGCATCCTCGTGCTCCT |
| | | | | GTTTACCTTGGT |
| | | | | GCAGAGGAGCAATGGGGAGTGTAAAGAGGCACTAGCAAAGTCCGAGATGAA |
| | | | | TGTGAATATGAA |
| | | | | GTATCAGCTTCCCAACTTCACCGCGGAAACACCCATCCAGAATGTCATTCTACA |
| | | | | TGAGCATCACAT |
| | | | | TTTCCTTGGTGCCACTAACTACATTTATGTTTTAAATGAGGAAGACCTTCAGAA |
| | | | | GGTTGCTGAGTA |
| | | | | CAAGACTGGGCCTGTGCTGGAACACCCAGATTGTTTCCCATGTCAGGACTGCA |
| | | | | GCAGCAAAGCC |
| | | | | AATTTATCAGGAGGTGTTTGGAAAGATAACATCAACATGGCTCTAGTTGTCGA |
| | | | | CACCTACTATGAT |
| | | | | GATCAACTCATTAGCTGTGGCAGCGTCAACAGAGGGACCTGCCAGCGACATG |
| | | | | TCTTTCCCCACAA |
| | | | | TCATACTGCTGACATACAGTCGGAGGTTCACTGCATATTCTCCCCACAGATAGA |
| | | | | AGAGCCCAGCC |
| | | | | AGTGTCCTGACTGTGTGGTGAGCGCCCTGGGAGCCAAAGTCCTTTCATCTGTA |
| | | | | AAGGACCGGTTC |
| | | | | ATCAACTTCTTTGTAGGCAATACCATAAATTCTTCTTATTTCCCAGATCATCCAT |
| | | | | TGCATTCGATATC |
| | | | | AGTGAGAAGGCTAAAGGAAACGAAAGATGGTTTTATGTTTTTGACGGACCAG |
| | | | | TCCTACATTGATG |
| | | | | TTTTACCTGAGTTCAGAGATTCTTACCCCATTAAGTATGTCCATGCCTTTGAAA |
| | | | | GCAACAATTTTAT |
| | | | | TTACTTCTTGACGGTCCAAAGGGAAACTCTAGATGCTCAGACTTTTCACACAAG |
| | | | | AATAATCAGGTT |
| | | | | CTGTTCCATAAACTCTGGATTGCATTCCTACATGGAAATGCCTCTGGAGTGTAT |
| | | | | TCTCACAGAAAA |
| | | | | GAGAAAAAAGAGATCCACAAAGAAGGAAGTGTTTAATATACTTCAGGCTGCG |
| | | | | TATGTCAGCAAG |
| | | | | CCTGGGGCCCAGCTTGCTAGACAAATAGGAGCCAGCCTGAATGATGACATTCT |
| | | | | TTTCGGGGTGTT |
| | | | | CGCACAAAGCAAGCCAGATTCTGCCGAACCAATGGATCGATCTGCCATGTGTG |
| | | | | CATTCCCTATCA |
| | | | | AATATGTCAACGACTTCTTCAACAAGATCGTCAACAAAAACAATGTGAGATGT |
| | | | | CTCCAGCATTTTT ACGGACCCAATCATGAGCACTGCTTTAATAGG |
| 4233 | MET | HSX2_01055597 | 484 | ACACTTCTGAGAAATTCATCAGGCTGTGAAGCGCGCCGTGATGAATATCGAAC |
| | | | | AGAGTTTACCAC |
| | | | | AGCTTTGCAGCGCGTTGACTTATTCATGGGTCAATTCAGCGAAGTCCTCTTAAC |
| | | | | ATCTATATCCAC |
| | | | | CTTCATTAAAGGAGACCTCACCATAGCTAATCTTGGGACATCAGAGGGTCGCT |
| | | | | TCATGCAG |
| 4233 | MET | HSX2_01055603 | 485 | GGTTTCTCGATCAGGACCATCAACCCCTCATGTGAATTTTCTCCTGGACTCCCA |
| | | | | TCCAGTGTCTCC |
| | | | | AGAAGTGATTGTGGAGCATACATTAAACCAAAATGGCTACACACTGGTTATCA |
| | | | | CTGGGAAGAAG |
| 4233 | MET | HSX2_01055610 | 486 | TGTGGCTGGTGCCACGACAAATGTGTGCGATCGGAGGAATGCCTGAGCGGGA |
| | | | | CATGGACTCAAC AGATCTGTCTGCCTGCAATCTACAAG |
| 4233 | MET | HSX2_01055633 | 487 | TGTGTCAAACAGTATTCTTGAATGTTATACCCCAGCCCAAACCATTTCAACTGA |
| | | | | GTTTGCTGTTAA |
| | | | | ATTGAAAATTGACTTAGCCAACCGAGAGACAAGCATCTTCAGTTACCGTGAAG |
| | | | | ATCCCATTGTCTA TGAAATTCATCCAACCAAATCTTTTATTAG |
| 4233 | MET | HSX2_01055665 | 488 | ATCAGTTTCCTAATTCATCTCAGAACGGTTCATGCCGACAAGTGCAGTATCCTC |
| | | | | TGACAGACATGT |
| | | | | CCCCCATCCTAACTAGTGGGGACTCTGATATATCCAGTCCATTACTGCAAAATA |
| | | | | CTGTCCACATTG |
| | | | | ACCTCAGTGCTCTAAATCCAGAGCTGGTCCAGGCAGTGCAGCATGTAGTGATT |
| | | | | GGGCCCAGTAGCCTGATTGTGCATTTCAATGAAGTCATAGGAAGAG |
| 4233 | MET | HSX2_01055670 | 489 | GGCATTTTGGTTGTGTATATCATGGGACTTTGTTGGACAATGATGGCAAGAAA |
| | | | | ATTCACTGTGCT GTGAAATCCTTGAACA |
| 4233 | MET | HSX2_01055680 | 490 | AATCCAACTGTAAAAGATCTTATTGGCTTTGGTCTTCAAGTAGCCAAAGGCAT |
| | | | | GAAATATCTTGCA |
| | | | | AGCAAAAAGTTTGTCCACAGAGACTTGGCTGCAAGAAACTGTAT |
| 4233 | MET | HSX2_01055701 | 491 | ATATGAAGTAATGCTAAAATGCTGGCACCCTAAAGCCGAAATGCGCCCATCCT |
| | | | | TTTCTGAACTGG |
| | | | | TGTCCCGGATATCAGCGATCTTCTCTACTTTCATTGGGGAGCACTATGTCCATG |
| | | | | TGAACGCTACTT |
| | | | | ATGTGAACGTAAAATGTGTCGCTCCGTATCCTTCTCTGTTGTCATCAGAAGATA |
| | | | | ACGCTGATGATG |
| | | | | AGGTGGACACACGACCAGCCTCCTTCTGGGAGACATCATAGTGCTAGTACTAT |
| | | | | GTCAAAGCAACA |
| | | | | GTCCACACTTTGTCCAATGGTTTTTTCACTGCCTGACCTTTAAAAGGCCATCGAT |
| | | | | ATTCTTTGCTCT |
| | | | | TGCCAAAATTGCACTATTATAGGACTTGTATTGTTATTTAAATTACTGGATTCTA |
| | | | | AGGAATTTCTTA |
| | | | | TCTGACAGAGCATCAGAACCAGAGGCTTGGTCCCACAGGCCACGGACCAATG |
| | | | | GCCTGCAGCCGT GACAACACTCCTGTCATATTGGAGT |
| 4233 | MET | HSX2_01055719 | 492 | ACCAAATCTTTTATTAGTGGTGGGAGCACAATAA |
| 4233 | MET | HSX2_02508602 | 493 | ATAAACCTCTCATAATGAAGGCCCCCGCTGTGCTTGCACCTGGCATCCTCGTGC |
| | | | | TCCTGTTTACCT |
| | | | | TGGTGCAGAGGAGCAATGGGGAGTGTAAAGAGGCACTAGCAAAGTCCGAGA |
| | | | | TGAATGTGAATA |
| | | | | TGAAGTATCAGCTTCCCAACTTCACCGCGGAAACACCCATCCAGAATGTCATTC |
| | | | | TACATGAGCATC |
| | | | | ACATTTTCCTTGGTGCCACTAACTACATTTATGTTTTAAATGAGGAAGACCTTC |
| | | | | AGAAGGTTGCTG |
| | | | | AGTACAAGACTGGGCCTGTGCTGGAACACCCAGATTGTTTCCCATGTCAGGAC |
| | | | | TGCAGCAGCAAA |
| | | | | GCCAATTTATCAGGAGGTGTTTGGAAAGATAACATCAACATGGCTCTAGTTGT |
| | | | | CGACACCTACTAT |
| | | | | GATGATCAACTCATTAGCTGTGGCAGCGTCAACAGAGGGACCTGCCAGCGAC |
| | | | | ATGTCTTTCCCCA |
| | | | | CAATCATACTGCTGACATACAGTCGGAGGTTCACTGCATATTCTCCCCACAGAT |
| | | | | AGAAGAGCCCA |
| | | | | GCCAGTGTCCTGACTGTGTGGTGAGCGCCCTGGGAGCCAAAGTCCTTTCATCT |
| | | | | GTAAAGGACCG |
| | | | | GTTCATCAACTTCTTTGTAGGCAATACCATAAATTCTTCTTATTTCCCAGATCAT |
| | | | | CCATTGCATTCG |
| | | | | ATATCAGTGAGAAGGCTAAAGGAAACGAAAGATGGTTTTATGTTTTTGACGG |
| | | | | ACCAGTCCTACAT |
| | | | | TGATGTTTTACCTGAGTTCAGAGATTCTTACCCCATTAAGTATGTCCATGCCTTT |
| | | | | GAAAGCAACAA |
| | | | | TTTTATTTACTTCTTGACGGTCCAAAGGGAAACTCTAGATGCTCAGACTTTTCA |
| | | | | CACAAGAATAAT |
| | | | | CAGGTTCTGTTCCATAAACTCTGGATTGCATTCCTACATGGAAATGCCTCTGGA |
| | | | | GTGTATTCTCAC |
| | | | | AGAAAAGAGAAAAAAGAGATCCACAAAGAAGGAAGTGTTTAATATACTTCAG |
| | | | | GCTGCGTATGTC |
| | | | | AGCAAGCCTGGGGCCCAGCTTGCTAGACAAATAGGAGCCAGCCTGAATGATG |
| | | | | ACATTCTTTTCGG |
| | | | | GGTGTTCGCACAAAGCAAGCCAGATTCTGCCGAACCAATGGATCGATCTGCCA |
| | | | | TGTGTGCATTCC |
| | | | | CTATCAAATATGTCAACGACTTCTTCAACAAGATCGTCAACAAAAACAATGTGA |
| | | | | GATGTCTCCAGC ATTTTTACGGACCCAATCATGAGCACTGCTTTAATAGG |
| 196410 | METTL7B | HSX2_02278211 | 494 | AAAAGTCATTGAAGAGCTTGTGGGGCTGTGGGACCTGCGCCCTCTGGAGGAA |
| | | | | TTCCATACACCC |
| | | | | ACTCAACTCTGGCAAATAGGAAATTGTCAAGTAGGAGACAAGGAGCAAAGTC |
| | | | | CTATCACAGCGG |
| | | | | GAGGGGACGCCAGCGCCTGCAGAGGCTGAGCAGGGAAAAAGCCAGTGCCCC |
| | | | | AGCGGAAGCAC |
| | | | | AGCTCAGAGCTGGTCTGCCATGGACATCCTGGTCCCACTCCTGCAGCTGCTGG |
| | | | | TGCTGCTTCTTAC |
| | | | | CCTGCCCCTGCACCTCATGGCTCTGCTGGGCTGCTGGCAGCCCCTGTGCAAAA |
| | | | | GCTACTTCCCCTA |
| | | | | CCTGATGGCCGTGCTGACTCCCAAGAGCAACCGCAAGATGGAGAGCAAGAAA |
| | | | | CGGGAGCTCTTC |
| | | | | AGCCAGATAAAGGGGCTTACAGGAGCCTCCGGGAAAGTGGCCCTACTGGAGC |
| | | | | TGGGCTGCGGA |
| | | | | ACCGGAGCCAACTTTCAGTTCTACCCACCGGGCTGCAGGGTCACCTGCCTAGA |
| | | | | CCCAAATCCCCA |
| | | | | CTTTGAGAAGTTCCTGACAAAGAGCATGGCTGAGAACAGGCACCTCCAATATG |
| | | | | AGCGGTTTGTG |
| | | | | GTGGCTCCTGGAGAGGACATGAGACAGCTGGCTGATGGCTCCATGGATGTGG |
| | | | | TGGTCTGCACTC |
| | | | | TGGTGCTGTGCTCTGTGCAGAGCCCAAGGAAGGTCCTGCAGGAGGTCCGGAG |
| | | | | AGTACTGAGACC G |
| 4240 | MFGE8 | 4240.004.1-D | 495 | CTGGCAGCAGTAAGATCTTCCCTGGCAA |
| 4240 | MFGE8 | 4240.004.1-F | 496 | CACCCAGCAGCAATGACGATAACCCCTGGATCCAGGTGAACCTGCTGCGGAG |
| | | | | GATGTGGGTAAC |
| | | | | AGGTGTGGTGACGCAGGGTGCCAGCCGCTTGGCCAGTCATGAGTACCTGAAG |
| | | | | GCCTTCAAGGTG |
| | | | | GCCTACAGCCTTAATGGACACGAATTCGATTTCATCCATGATGTTAATAAAAAA |
| | | | | CACAAGGAGTTT |
| | | | | GTGGGTAACTGGAACAAAAACGCGGTGCATGTCAACCTGTTTGAGACCCCTGT |
| | | | | GGAGGCTCAGT |
| | | | | ACGTGAGATTGTACCCCACGAGCTGCCACACGGCCTGCACTCTGCGCTTTGAG |
| | | | | CTACTGGGCTGT |
| | | | | GAGCTGAACGGATGCGCCAATCCCCTGGGCCTGAAGAATAACAGCATCCCTG |
| | | | | ACAAGCAGATCA |
| | | | | CGGCCTCCAGCAGCTACAAGACCTGGGGCTTGCATCTCTTCAGCTGGAACCCC |
| | | | | TCCTATGCACGG |
| | | | | CTGGACAAGCAGGGCAACTTCAACGCCTGGGTTGCGGGGAGCTACGGT |
| 4240 | MFGE8 | HSX2_01056073 | 497 | GGGCTTGCATCTCTTCAGCTGGAACCCCTCCTATGCACGGCTGGACAAGCAGG |
| | | | | GCAACTTCAACG |
| | | | | CCTGGGTTGCGGGGAGCTACGGTAACGATCAGTGGCTGCAG |
| 4240 | MFGE8 | HSX2_01056081 | 498 | CCAGGGGGCCCGTAACTTTGGCTCTGTCCAGTTTGTGGCATCCTA |
| 4240 | MFGE8 | HSX2_01056085 | 499 | GTGACAGGCATCATCACCCAGGGGGCCCGTAACT |
| 4240 | MFGE8 | HSX2_01056092 | 500 | TTCCCTGGCAACTGGGACAACCACTCCCACAAGAAGAACTTGTTTGAGACGCC |
| | | | | CATCCTGGCTCG |
| | | | | CTATGTGCGCATCCTGCCTGTAGCCTGGCACAACCGCATCGCCCTGCGCCTGG |
| | | | | AGCTGCTGGGCT GTTAGTGGCCAC |
| 4240 | MFGE8 | HSX2_01056094 | 501 | CCACCCCCAGGTCTTCCTGCTTTCCATGGGCCCGCTGCCTCTTGGCTTCTCAGC |
| | | | | CCCTTTAAATCAC CATAGGGCTGGGGACTGGGGAAGGGGAGG |
| 4240 | MFGE8 | HSX2_01056095 | 502 | GTGTTCAGAGGCAGCACCACCACACAGTCACCCCTCCCTCCCTCTTTCCCACCC |
| | | | | TCCACCTCTCAC |
| | | | | GGGCCCTGCCCCAGCCCCTAAGCCCCGTCCCCTAACCCCCAGTCCTCACTGTCC |
| | | | | TGTTTTCTTAGG |
| | | | | CACTGAGGGATCTGAGTAGGTCTGGGATGGACAGGAAAGGGCAAAGTAGGG |
| | | | | CGTGTGGTTTCC |
| | | | | CTGCCCCTGTCCGGACCGCCGATCCCAGGTGCGTGTGTCTCTGTCTCTCCTAGC |
| | | | | CCCTCTCTCACA |
| | | | | CATCACATTCCCATGGTGGCCTCAAGAAAGGCCCGGAAGCGCCAGGCTGGAG |
| | | | | ATAACAGCCTCT |
| | | | | TGCCCGTCGGCCCTGCGTCGGCCCTGGGGTACCATGTGGCCACAACTGCTGTG |
| | | | | GCCCCCTGTCCC |
| | | | | CAAGACACTTCCCCTTGTCTCCCTGGTTGCCTCTCTTGCCCCTTGTCCTGAAGCC |
| | | | | CAGCGACACAG |
| | | | | AAGGGGGTGGGGCGGGTCTATGGGGAGAAAGGGAGCGAGGTCAGAGGAGG |
| | | | | GCATGGGTTGG CAGG |
| 4240 | MFGE8 | HSX2_01056096 | 503 | GTGGGCGTTTGGGGCCCTCTATGCTGGCTTTTCACCCCAGAGGACACAGGCAG |
| | | | | CTTCCAAAATAT ATTTATCTTCTTCACGGGAA |
| 4240 | MFGE8 | HSX2_01056103 | 504 | GGACTGGCAGCAGTAAGATCTTCCCTGGCAACTG |
| 4240 | MFGE8 | HSX2_02508672 | 505 | ATCTTCCCTGGCAACTGGGACAACCACTCCCACAAGAAGAACTTGTTTGAGAC |
| | | | | GCCCATCCTGGC |
| | | | | TCGCTATGTGCGCATCCTGCCTGTAGCCTGGCACAACCGCATCGCCCTGCGCCT |
| | | | | GGAGCTGCTGG |
| | | | | GCTGTTAGTGGCCACCTGCCACCCCCAGGTCTTCCTGCTTTCCATGGGCCCGCT |
| | | | | GCCTCTTGGCTT |
| | | | | CTCAGCCCCTTTAAATCACCATAGGGCTGGGGACTGGGGAAGGGGAGGGTGT |
| | | | | TCAGAGGCAGC |
| | | | | ACCACCACACAGTCACCCCTCCCTCCCTCTTTCCCACCCTCCACCTCTCACGGGC |
| | | | | CCTGCCCCAGC |
| | | | | CCCTAAGCCCCGTCCCCTAACCCCCAGTCCTCACTGTCCTGTTTTCTTAGGCACT |
| | | | | GAGGGATCTGA |
| | | | | GTAGGTCTGGGATGGACAGGAAAGGGCAAAGTAGGGCGTGTGGTTTCCCTGC |
| | | | | CCCTGTCCGGAC |
| | | | | CGCCGATCCCAGGTGCGTGTGTCTCTGTCTCTCCTAGCCCCTCTCTCACACATC |
| | | | | ACATTCCCATGG |
| | | | | TGGCCTCAAGAAAGGCCCGGAAGCGCCAGGCTGGAGATAACAGCCTCTTGCC |
| | | | | CGTCGGCCCTGC |
| | | | | GTCGGCCCTGGGGTACCATGTGGCCACAACTGCTGTGGCCCCCTGTCCCCAAG |
| | | | | ACACTTCCCCTT |
| | | | | GTCTCCCTGGTTGCCTCTCTTGCCCCTTGTCCTGAAGCCCAGCGACACAGAAGG |
| | | | | GGGTGGGGCG |
| | | | | GGTCTATGGGGAGAAAGGGAGCGAGGTCAGAGGAGGGCATGGGTTGGCAG |
| | | | | GGTGGGCGTTTG |
| | | | | GGGCCCTCTATGCTGGCTTTTCACCCCAGAGGACACAGGCAGCTTCCAAAATA |
| | | | | TATTTATCTTCTT CACGGGAA |
| 4257 | MGSTT | HSX2_01057030 | 506 | GTTTTTGCCAATCCAGAAGACTGTGTAGCATTTGGCAAAGGAGAAAATGCCAA |
| | | | | GAAGTATCTTCGAACAGATGACAGAGTAGAACGTGTAC |
| 4281 | MID1 | HSX2_01057790 | 507 | ACTGTAAAGGATCAGAAAGTCATTGGCAGAGAGAAGCAGCAGTATTACCCTA |
| | | | | GGAATAGGAGAA |
| | | | | AGTGGGTCAGCAACTCCCTACAGGCAAGACTGATGGGGAGAAGATGCATTTC |
| | | | | AAGACGCGAAAGAAAACTGACTCTAGATGTAGAGAAG |
| 54471 | MIEF1 | HSX2_01794393 | 508 | GTGGTGACAGCTGACCACATCCAACTCATTGTGCCCCTTGTGCTGGAGCAGAA |
| | | | | CCTGTGGTCATG |
| | | | | TATTCCTGGTGAAGACACCATCATGAATGTCCCTGGCTTCTTCCTGGTGCGTCG |
| | | | | TGAGAATCCAGA |
| | | | | GTACTTTCCTCGTGGGAGCAGTTACTGGGACCGCTGTGTAGTAGGGGGCTACC |
| | | | | TCTCTCCAAAGA |
| | | | | CAGTCGCAGATACATTTGAGAAGGTAGTGGCTGGCTCCATCAATTGGCCAGCC |
| | | | | ATAGGGTCCCTC TTGGACTATGTGATCCGCCCGGCCCCACCCCCAGAAG |
| 10962 | MLLTT1 | HSX2_01539260 | 509 | GTACTGGGTCACAGGGATTCCTCCTTTCCCCCCCAAATATTAACTCCAGAAACT |
| | | | | AGGCCTGACTGG |
| | | | | GGACACCTGAGAGTAGTATAGTAGTGCAAAATGGAAGACTGATTTTTGACTCT |
| | | | | ATTATAATCAGC |
| | | | | TTCAGAGATTCCTTAAACCTTCCTAATTTCCTGCTCCAGGGCAGTAAACACAAA |
| | | | | TATTTCTTCAAGG |
| | | | | GGTGATGAAAACCTCGGAAGTTTTAATTTGAGGTTATCTGCTACGAAACAGTA |
| | | | | TTTCTAAAAGGCT |
| | | | | AAAGTGATAAGTCTCTTGCTTTTTTTTGATCCTGCTCTTATATTCTTTTTTTTCCT |
| | | | | CAGAGAAATCAG |
| | | | | GAGGGTAGTTAGAGGTATAAAACAGGAGGAAATATTATGGAAAATGAAAATA |
| | | | | GGGAAAATAATT |
| | | | | GAATCATTTTAGAAGTAGCTAATTTCTTTTCTCAAAAGAGTGTCCCTTCTTCACA |
| | | | | CCTACTCACTTT |
| | | | | ACAACTTTGCTCCTAACTGTGGGTTGAAAACTCTAGCTAAAGAAAGTTATCAA |
| | | | | ATCTTAACATGCA |
| | | | | TTCCTACTATTATGATAGTTTTTAAGGTTTCAATTCAATCTTCTGAACGGCATAA |
| | | | | GTCCTATTTTAG |
| | | | | CCTTACCTCCTGCATTTGCAATACGTAATACTGATCAGTGGGCACAGTTCTTCA |
| | | | | GCTACATTGAGA |
| | | | | CCCTGAAATGAACAATTATATTCTGACTCGACATCTTGTCCCCAATCCTTCCAAA |
| | | | | AATATTGATGGT |
| | | | | GATTTGTGCTACCATTTACTCGTTTATTTAATAAAGACATTCAATCCCA |
| 4301 | MLLT4 | HSX2_01059895 | 510 | AGTAGAGTTAAGCCCAGATGGTTCTGACTCTAGA |
| 4301 | MLLT4 | HSX2_01059916 | 511 | CTGAAGATTCATTTTTGTCTGCCATTATAAATTATACTAATAGCTCTACAGTCCA |
| | | | | CTTTAAGTTGTC |
| | | | | CCCTACATATGTATTATATATGGCATGCCGGTATGTATTGTCCAACCAGTACAG |
| | | | | ACCTGACATCAG |
| | | | | CCCTACAGAGCGCACACATAAAGTCATTGCAGTCGTCAACAAGATGGTGAGCA |
| | | | | TGATGGAGGGT GTCATCCAG |
| 4301 | MLLT4 | HSX2_01060018 | 512 | GACCAAACTCTTACCCAGGATCTACTGGAGCAGCTGTTGGAGCCCATGACGCC |
| | | | | TGTCGGGATGCA AAAGAGAAGCGCTCTAAAAG |
| 4301 | MLLT4 | HSX2_01060025 | 513 | GAGAAGCGCTCTAAAAGCCAAGATGCAGATTCAC |
| 4301 | MLLT4 | HSX2_02508915 | 514 | AGGTGCTGCAGATGATGGATGGACGTCTAGCTGC |
| 84954 | MPND | HSX2_02118356 | 515 | ATCTCCTTGGCCAGCAGGACGCCCAAGGACCAGAGCCTGTGTCACGTCCTGGA |
| | | | | ACAGGTGTGCG |
| | | | | GCGTCCTCAAGCAGGGGAGCTGAGCCTTCCAGGGCAGGGTGGGCTCCAGTTG |
| | | | | TCTTGAGGGTCC GGATGGGCTCAGGTAATAAAGAAACGGAAGCAGCA |
| 10205 | MPZL2 | HSX2_01479681 | 516 | CTCTTTGGCCTATAGCAGCTGTGGAAATTTATACCTCCCGGGTGCTGGAGGCT |
| | | | | GTTAATGGGACA |
| | | | | GATGCTCGGTTAAAATGCACTTTCTCCAGCTTTGCCCCTGTGGGTGATGCTCTA |
| | | | | ACAGTGACCTGGAATTTTCGTCCTCTAGACGGGGGACCTGAGCAGTTT |
| 10205 | MPZL2 | HSX2_01479685 | 517 | GTATTCTACTACCACATAGATCCCTTCCAACCCATGAGTGGGCGGTTTAAGGAC |
| | | | | CGGGTGTCTTG |
| | | | | GGATGGGAATCCTGAGCGGTACGATGCCTCCATCCTTCTCTGGAAACTGCAGT |
| | | | | TCGACGACAATG |
| | | | | GGACATACACCTGCCAGGTGAAGAACCCACCTGATGTTGATGGGGTGATAGG |
| | | | | GGAGATCCGGCT CAGCGTCGTGCACACTG |
| 10205 | MPZL2 | HSX2_01479690 | 518 | TACGCTTCTCTGAGATCCACTTCCTGGCTCTGGCCATTGGCTCTGCCTGTGCAC |
| | | | | TG |
| 10205 | MPZL2 | HSX2_01479691 | 519 | ATGATCATAATAGTAATTGTAGTGGTCCTCTTCCAGCATTACCGGAAAAAGCG |
| | | | | ATGGGCCGAAAG AGCTCATAAAGTGGTGGAGATAAAATC |
| 10205 | MPZL2 | HSX2_01479697 | 520 | AAAAGAAGAGGAAAGGCTCAACCAAGAGAAAAAGGTCTCTGTTTATTTAGAA |
| | | | | GACACAGACTAA CAATTTTAGATG |
| 10205 | MPZL2 | HSX2_01479701 | 521 | GAAGCTGAGATGATTTCCAAGAACAAGAACCCTAGTATTTCTTGAAGTTAATG |
| | | | | GAAACTTTTCTTT |
| | | | | GGCTTTTCCAGTTGTGACCCGTTTTCCAACCAGTTCTGCAGCATATTAGATTCT |
| | | | | AGACAAGCAACA |
| | | | | CCCCTCTGGAGCCAGCACAGTGCTCCTCCATATCACCAGTCATACACAGCCTCA |
| | | | | TTATTAAGGTCT |
| | | | | TATTTAATTTCAGAGTGTAAATTTTTTCAAGTGCTCATTAGGTTTTATAAACAAG |
| | | | | AAGCTACATTTT |
| | | | | TGCCCTTAAGACACTACTTACAGTGTTATGACTTGTATACACATATATTGGTAT |
| | | | | CAAAAGGGATAA |
| | | | | AAGCCAATTTGTCTGTTACATTTCCTTTCACGTATTTCTTTTAGCAGCACTTCTG |
| | | | | CTACTAAAGTTAA |
| | | | | TGTGTTTACTCTCTTTCCTTCCCACATTCTCAATTAAAAGGTGAGCTAAGCCTCC |
| | | | | TCGGTGTTTCTG |
| | | | | ATTAACAGTAAATCCTAAATTCAAACTGTTAAATGACATTTTTATTTTTATGTCT |
| | | | | CTCCTTAACTATG |
| | | | | AGACACATCTTGTTTTACTGAATTTCTTTCAATATTCCAGGTGATAGATTTTTGT |
| | | | | TGTTTTGTTAATT |
| | | | | AATCCAAGATTTACAATAGCACAACGCTAAATCACACAGTAACTACAAAAGGT |
| | | | | TACATAGATATG |
| | | | | AAAAGATTGGCAGAGGCCATTGCAGGATGAATCACTTGTCACTTTTCTTCTGT |
| | | | | GCTGGGAAAAAT |
| | | | | AATCAACAATGTGGGTCTTTCATGAGCAGTGACGGATAGTTTAGCTTACTATG |
| | | | | TTTCCCCCCCAAT |
| | | | | TCAATGATCTATAACAACAGAGCAAAGTCTATGCTCATTTGCAGACTGGAATC |
| | | | | ATTAAGTAATTTA |
| | | | | ATAAAAAAATTGTGAAACAGCATATTACAAGTTTGAAAATTCAGGGCTGGTGA |
| | | | | AAAAAATCAACT |
| | | | | CTAAATGATGATAATTTTGTACAGTTTTATATAAAACTCTGAGAACTAGAAGAA |
| | | | | ATTATTAACTTTT |
| | | | | TTTCTTTTTTAATTCTAATTCACTTGTTTATTTTGGGGGAGGAAGACTTTGGTAT |
| | | | | GGAGCAAAGAA |
| | | | | ATACCAAAACTACTTTAAATGGAATAAAACCAACTTTATTCTTTTTTTCCCCCAT |
| | | | | ACTGGTAGATAA |
| | | | | AGCAAACTTTATAAGTGGGCTATTGAAAGAAAAGTTACAAGCTTAAGATACAG |
| | | | | AAGCATTTGTTC |
| | | | | AAAGGATAGAAAGCATCTAAAAGTTTAGGCTCAAGATCAATCTTTACAGATTG |
| | | | | ATATTTTCAGTTT |
| | | | | TTAATCGACTGGACTGCAGATGTTTTTTCTTTTAACAAACTGGAATTTTCAAAC |
| | | | | AGATTATCTGTAT |
| | | | | TTAAATGTATAGACCTTGATATTTTTCCAATACTATTTTTTAAAAAATTGTATGA |
| | | | | TTTACATATGAAC |
| | | | | CTCAGTTCTGAAATTCATTACATATCTGTCTCATTCTGCCTTTTATACTGTCTAAA |
| | | | | AAAGCAAAGTT |
| | | | | TTAAAGTGCAATTTTAAAACTGTAAATTACATCTGAAGGCTATATATCCTTTAA |
| | | | | TCACATTTTATAT |
| | | | | TTTTTCTTCACAATTCTAACCTTTGAAAATATTATAACTGGATATTTCTTCAAAC |
| | | | | AGATGTCCTGGAT |
| | | | | GATGGTCCATAAGAATAATGAAGAAGTAGTTAAAAATGTATGGACAGTTTTTC |
| | | | | CGGCAAAATTTG |
| | | | | TAGCTTATGTCTTGGCTAAATAGTCAAGGGGTAATATGGGCCTGTTGTTTAGT |
| | | | | GTCTCCTTCCTAA |
| | | | | AGAGCACTTTTGTATTGTAATTTATTTTTTATTATGCTTTAAACACTATGTAAAT |
| | | | | AAACCTTTAGTAA TAAAGAATTATCAGTTATATAA |
| 10205 | MPZL2 | HSX2_01479703 | 522 | ACTAACAATTTTAGATGGAAGCTGAGATGATTTC |
| 10205 | MPZL2 | HSX2_01479705 | 523 | GTAAGGTTCACAAATAGGTTGATTTCTTTCTTCAGCTTTCTGACATGTCCAGCC |
| | | | | CATCTCTAATGAG |
| | | | | GACTCCCAGATCATCACTTTATGGCTGTTAGGTGTTTCCCATATGAAATTAGAG |
| | | | | GAGCTGGGTCA |
| | | | | GGGAGACAAAAGTCTTCTATTAGTCTTATGGATAGCTCCTCCTTGAGTGTATTT |
| | | | | TGTGCAAAAGAT |
| | | | | TAAGAAGCTGGACTCTACTGCCATTAAAGCTGAGAGAATCCTAAGGTTATTTG |
| | | | | TGGCTTCGGGGT |
| | | | | TATATTTATTACTACTACTACTAATAAATATTCAACAAGTAAATAAATCTTTTTT |
| | | | | AAATCAA |
| 10205 | MPZL2 | HSX2_01479706 | 524 | ACTAACAATTTTAGATGGTAAGGTTCACAAATAG |
| 10205 | MPZL2 | HSX2_02534823 | 525 | TACGCTTCTCTGAGATCCACTTCCTGGCTCTGGCCATTGGCTCTGCCTGTGCAC |
| | | | | TGATGATCATAA |
| | | | | TAGTAATTGTAGTGGTCCTCTTCCAGCATTACCGGAAAAAGCGATGGGCCGAA |
| | | | | AGAGCTCATAAA GTGGTGGAGATAAAATC |
| 9902 | MRC2 | HSX2_01451912 | 526 | GGGGCCCCAGAGGAGCAGGAGTCTGGGAGGGCCCAGAGTTCACCCTCTAGT |
| | | | | GGATCCAGGAGG |
| | | | | AGCAGCACCCGAGCCCTGGAGTGGCCCAGTACCCTTCCAAGAGGCCACAGTC |
| | | | | CCAGCCAGGACA |
| | | | | AAGTATGCGGCCCATCCTGGTGCGACAGCGTGGGACAATGTGAACATGGACT |
| | | | | CGAAGACATGGC |
| | | | | CCTTTCTCTGTAGTTGATTTTTTAAATGTGCCATTATTGTTTTTAA |
| 4524 | MTHFR | HSX2_01066737 | 527 | GACGAGGCCTTTGCCCTGTGGATTGAGCGGTGGGGAAAGCTGTATGAGGAG |
| | | | | GAGTCCCCGTCCC |
| | | | | GCACCATCATCCAGTACATCCACGACAACTACTTCCTGGTCAACCTGGTGGACA |
| | | | | ATGACTTCCCAC |
| | | | | TGGACAACTGCCTCTGGCAGGTGGTGGAAGACACATTGGAGCTTCTCAACAG |
| 9617 | MTRF1 | HSX2_01419835 | 528 | GTGGATGTGAAATTGGACCCCAAGGATTTGCGAATAGATACATTTCGAGCCAA |
| | | | | AGGAGCAGGAG |
| | | | | GGCAGCATGTTAATAAAACTGATAGTGCCGTCAGACTTGTCCACATCCCCACA |
| | | | | G |
| 54587 | MXRA8 | HSX2_02554391 | 529 | GAAGGATGTTAACTTGGCGGAGTTCGCTGTGGCTGCAGGGGACCAGATGCTT |
| | | | | TACAGGAGTGAGGACATCCAGCTAG |
| 23077 | MYCBP2 | HSX2_01593794 | 530 | GGGATGTCCCACTCTTGTTCAAGCATTGCCAGGCCCTAGCACACAAGTCACTG |
| | | | | CAGGCAGCAACC |
| | | | | ATACGGCAGTACTTTTAATGGATGGACAGGTCTTCACATTTGGAAGTTTTTCT |
| 4628 | MYH10 | HSX2_01073103 | 531 | TGAAACACTCCATAAGAACAATGCCCTTAAAGTTGTGCGAGAGCTACAAGCCC |
| | | | | AAATTGCTGAAC |
| | | | | TTCAGGAAGACTTTGAATCCGAGAAGGCTTCACGGAACAAGGCCGAAAAGCA |
| | | | | GAAAAGGGACTT |
| | | | | GAGTGAGGAACTGGAAGCTCTGAAAACAGAGCTGGAGGACACGCTGGACAC |
| | | | | CACGGCAGCCCA GCAGGAACTACG |
| 4628 | MYH10 | HSX2_01073190 | 532 | CAATACAGTGGGACAGTTAGGAATGCACCCGGGGCCTCCTGCAGATTTCGGA |
| | | | | AATTGGCAAGCT |
| | | | | ACGGGATTCCTTCCTGAAAGATCAACTGTGTCTTAAGGCTCTCCAGCCTATGCA |
| | | | | TACTGTATCCTG |
| | | | | CTTCAGACTTAGGTACAATTGCTCCCCTTTTTATATATAGACACACACAGGACA |
| | | | | CATATATTAAACA |
| | | | | GATTGTTTCATCATTGCATCTATTTTCCATATAGTCATCAAGAGACCATTTTATA |
| | | | | AAACATGGTAAG |
| | | | | ACCCTTTTTAAAACAAACTCCAGGCCCTTGGTTGCGGGTCGCTGGGTTATTGG |
| | | | | GGCAGCGCCGTGGTCGTCACTCAGTCGCTCTGCA |
| 140469 | MYO3B | HSX2_02220753 | 533 | GTGTTTCAGCTCAACTCACCAGTACACGTCTGCGGAGAAACACATCTGTTGGC |
| | | | | ACCCCGTTCTGGATGGCCCCTGAG |
| 4646 | MYO6 | HSX2_01075718 | 534 | GGTACCTCTGAAAGTGGAGCAAGCAAACAATGCTCGTGATGCCCTGGCAAAG |
| | | | | ACAGTGTATAGC |
| | | | | CATCTTTTTGATCATGTGGTAAACAGAGTAAATCAGTGTTTTCCTTTTGAAACA |
| | | | | TCATCCTATTTTA TTGGAGTCCTAGATATTGCTGGTTTTG |
| 4664 | NAB1 | HSX2_01077841 | 535 | GAGAAAGACCTTTGAATCTCCGAATGCCTAATTTACAGAACAGACAACCCCAT |
| | | | | CATTTTGTGGTG |
| | | | | GATGGGGAGCTGAGCAGACTTTACCCCAGTGAGGCAAAGTCCCACTCATCAG |
| 4665 | NAB2 | HSX2_01077903 | 536 | AGTTCGAGGAAGGGCTGCTGGACAGATGTCCTGCCCCAGGACCCCATCCCGC |
| | | | | GCTGGTGGAGG |
| | | | | GTCGCAGGAGCAGCGTGAAAGTGGAGGCTGAGGCCAGCCGGCAGTGAGGGT |
| | | | | TGGACTGGTGTC |
| | | | | TTCAGACCCAGGACCTCAGACTTCTGGCTCACACAGACCCCCACGCTCTCCATC |
| | | | | CCCGGAATCTAG |
| | | | | TCACAACCCTGGATCCTTCCTCTGCCCTTCTCCTGCCTCCCCACCTGCTCCATGG |
| | | | | GCATAAGACTGT |
| | | | | GGGGCTTCAAGCAATAACAAGCAGAGGCCTGGAGAGAGGACACAAGGAGGG |
| | | | | TGCGTGGTGCCC |
| | | | | TCACCCCTGCCCAGAGCGAGGGGGCAAGGACTCTGCCTCCAGGGCATCTGGG |
| | | | | GTTTTCCCCTCCC |
| | | | | TCACACAACACACTCCCATTCTCTTTAGGTTTGCACCAGTGGTGTGAGCAGTTG |
| | | | | GACTCAGTTTGG |
| | | | | ACAAGGGGGAAAGGGGGACTTCCCTGGGAAGGTCCAGCTAAAAGTGGCAAC |
| | | | | ATTTGCCCCCAG |
| | | | | AATTGGGGGCCTGGGAACACTGGACCTGCTCCTTCTCCCCTCTCCTTCCCCGTT |
| | | | | TTTGTGCTTCTG |
| | | | | GTTTGTTTCTTTAATTAATTTAACAAGTGCTGCAGTTTGCCCTCCCATTCCCATC |
| | | | | TATCCCCCAAGTC |
| | | | | CTTTGCAATTTCTTCCCTGCCCTACATAGGGGCGGTGGGTGTGGGATCCCTTCA |
| | | | | CTGGCCCCCTCG |
| | | | | GGAGGCCTGGGTTGGACTCAGGGTCTCCTCCAGCTGGGGGCTGGACCGCAGC |
| | | | | ACCTATCTGAGC |
| | | | | AGTTAGAGCGTCTTTCTTTTCAGATTGTGTACAGTAGATTATTTATTTTGTTATT |
| | | | | TTGGAATAAAAT TTATTTTATGGCTTAGGA |
| 89797 | NAV2 | HSX2_02128541 | 537 | GAATTGCGCCAGTACCTGTCCAACCTTGCTGACCAGTGCAACAGTGAGAACAA |
| | | | | TGCTGTGGACAT |
| | | | | GCCCCTCGTCATCATCCTGGACAACCTACACCACGTGAGCTCTCTGGGAGAGA |
| | | | | TCTTCAATGGGC TGCTCAACTGCAAGTACCACAAATG |
| 26960 | NBEA | HSX2_01695002 | 538 | GAGGATATCATTGGCCACTCCTCGACAGCTTTATAAATCTTCCAATATGACTCA |
| | | | | GCGCTGGCAAAG |
| | | | | AAGGGAAATTTCAAACTTCGAATATTTGATGTTCCTTAATACTATTGCAG |
| 26960 | NBEA | HSX2_01695190 | 539 | AATCGCCACTGGGCTCGTCACGACAGCCTCCTTCAGCTCCAACCCGGCTAATTA |
| | | | | ATCCCCACTCAT GGATATAGGCACACAAACTAAAG |
| 29781 | NCAPH2 | HSX2_01722501 | 540 | AAGCGCAAGAGGAAGGGCGCTGCCAAGCTGCAGG |
| 23154 | NCDN | HSX2_01602288 | 541 | CTACCTGGGAGCCCTCCGTGAGGCCAAGAATGACAGCGAGCAGTTTGCAGCC |
| | | | | CTGCT |
| 400746 | NCMAP | HSX2_02394908 | 542 | ACCACTGTCCAAAGAGCCTCTCCAGAGTCAAGACCCAGAGGCACACTCTCTGG |
| | | | | CAGCTTCACAAT |
| | | | | GAGCTTCTTCTGGTCAGGTCGACAGAGACATCTTTGACGCAATCTCTGATGCTT |
| | | | | CCAGCAATCCTC |
| | | | | AACCTTGTCTGCCCTGCCCTACCCCAACTGTGTCCACATCCCTGCCGCCACCCC |
| | | | | ACCAAAAAGCTG |
| | | | | CAGAACATTCTTTTGTCATCTGATGAGGTAGAGCTATGTTGGGAATCCACCAAT |
| | | | | GTGGGCTTGGC |
| | | | | TTTCCCCCACACTGTAGTTAGACAGATAGACAGATAGCCCAGGAGCCAGGTGT |
| | | | | CAGGGAGCACT |
| | | | | GCTGAGAGTATCACAATAGGATCTGTCACGGGGTTCATATCAGATGAAGCGCC |
| | | | | GTATCCACTGCT |
| | | | | TCACAGAGCAAAACATTCAATCCCATAACCAGGCACAGGGGAACTAACTTGGA |
| | | | | CTAACTAACCAG |
| | | | | AAAACCTTGTTAACGTATAACTTGTTCCAGTACTACATCTCTGCCTGCTGGCTC |
| | | | | ATGACAATTGCTC |
| | | | | AGCACATTTTCCCCTCTTGAAGAAAGGTTGCAAGAAGAACTAAATTATCCTCAA |
| | | | | AAGATTTCTGCT |
| | | | | TCATTAGTAAAGAGTCAGTGATGGAATAGGGTGACTCTGCAGAATAGTGGCC |
| | | | | TCTAGGGTAGGA |
| | | | | GCTTGTTGTGTTGTCCGTGGGCCTGGAATGATCCTGGTGGCTGATCAGGGTCC |
| | | | | TTCTCCCACTCT |
| | | | | GGGCTGTATCAACCCTGACGGTCTTGGTCTTTGGCTCCCCTTTATCTGGATTCT |
| | | | | GAGCACGCTGAC |
| | | | | TGTCCTGTTAATGCCTTCCCTCCAAGGACCAGTATTTGGAGATTAATTAGATTA |
| | | | | CAACTCTATCTAT |
| | | | | GTTACCTTTGTCCTTCCTGGTCACCTTGCAGATTCAAGACATGTTCAAAGCAAC |
| | | | | ACATTCACAACC |
| | | | | CATTTCTATTCTATAGCAACCTCGTCTGTGACTCCTTAGCCTGGAGAACAATCT |
| | | | | ACCAAGAAGAGA |
| | | | | AAGTATCTGGAATTAAGAAGTCCTACCATCCAAGCCCTACTTCCTGGTTGTGTG |
| | | | | GCCTTGGAAAA |
| | | | | GTGACTCAACCTCTTTATATTCAGTTTCCTAACCATGAAGTGGAAATGATAACA |
| | | | | CCTGCCTCATTG |
| | | | | GGGCACTATAACAAGTGAAGGACTTAGGAAAACATCTGGAGTATAGCGCCTG |
| | | | | GCACCCAGGAGA |
| | | | | TGCTTAATAAATGGGAACCAGGATTCTTTTTCTTTTCTTTTTTCTTTTCTTTTTTTT |
| | | | | TTTTTTTGAGAC AGAGCCTCAATCTGTCACCTGG |
| 8202 | NCOA3 | HSX2_01324111 | 543 | CTCCTTTTCCCAGCAGCAGTTTGCCCACCAGGGGAATCCTGCAGTGTATAGTAT |
| | | | | GGTGCACATGA |
| | | | | ATGGCAGCAGTGGTCACATGGGACAGATGAACATGAACCCCATGCCCATGTCT |
| | | | | GGCATGCCTAT GGGTCCTGATCAG |
| 25915 | NDUFAF3 | HSX2_01668012 | 544 | GGTCCGGTTCGGCCTGGCAGGTGTTCGGGACGCG |
| 4753 | NELL2 | HSX2_01086049 | 545 | GGTATAATGCAAGATGTCCAATTACTTGTCATGCCCCAGGGATTTATTGCTCAG |
| | | | | TGCCCAGATCTT AATCGCA |
| 25825 | BACE2 | HSX2_01660260 | 546 | TTTACATTCAGCCCATGATGGGGGCCGGCCTGAATTATGAATGTTACCGATTC |
| | | | | GGCATTTCCCCAT |
| | | | | CCACAAATGCGCTGGTGATCGGTGCCACGGTGATGGAGGGCTTCTACGTCATC |
| | | | | TTCGACAGAGC CCAGAAGAGGGTGGGCTTCGCAGCGAGCCCCTGTGCAG |
| 4781 | NFIB | 4781.006.2-B | 547 | CCTGGAACTCTAGCACGCCGAGTGAACTTGAATCTTTGGCTATTTAAGGAGGA |
| | | | | CTGGGTTTGTTG |
| | | | | TGAAGTTGCGGTGATCCAGCGCAGAGCCCCGTCCTGATTGATCGCATCGCGG |
| | | | | GGCTCAGATGAC |
| | | | | TGTAAAATGAATAGATGAAATTCTTGCTTCTCGAAGATTTTCTTGGGCATCTCC |
| | | | | CGGAAAGTGCGT TTTAAGGCGAAGTCATGATGTATTCT |
| 4781 | NFIB | HSX2_01087767 | 548 | TTTGGCTATTTAAGGAGGACTGGGTTTGTTGTGA |
| 57185 | NIPAL3 | HSX2_01915059 | 549 | AAAAAAAAAAGTACTAGGTGCAAATAACATTATGAAGTGGTATTTAATTAAAA |
| | | | | ATCCATGCAGTG ACAGCTGA |
| 79159 | NOL12 | HSX2_02004046 | 550 | GATCTCCTCCCTCACAGCATCACTACATGCACACAGCCGCAAAAAGGTCAAGA |
| | | | | GGAAACATCCCC |
| | | | | GACGGGCCCAGGACTCCAAAAAGCCCCCAAGGGCCCCTCGTACCAGCAAGGC |
| | | | | CCAGCGCCGCC |
| | | | | GTCTCACAGGCAAAGCACGGCACAGCGGGGAGTGAGACCGAGAACGAAGCG |
| | | | | GTGCCCCAGTCT |
| | | | | AGGCTGCGGGGACCTGTCCTTGCTCAGCTTGGCTGTCCCTGTAGCCCAGCCTG |
| | | | | CACCTAG |
| 7064 | THOP1 | HSX2_01266163 | 551 | GAACCAACATGCCTGCAACCTTCGGCCATCTGGCAG |
| 7064 | THOP1 | HSX2_02521832 | 552 | GAACCAACATGCCTGCAACCTTCGGCCATCTGGCAGGTGGCTACGACGCCCAG |
| | | | | TACTACGGGTA |
| | | | | CCTGTGGAGCGAGGTGTATTCCATGGACATGTTCCACACGCGCTTCAAGCAGG |
| | | | | AGGGTGTCCTGAACAGCAAG |
| 124056 | NOX01 | HSX2_02190737 | 553 | CGGTCAGGCCAGCCTGGATGCACCACTGTTGGGA |
| 10577 | NPC2 | HSX2_01512515 | 554 | GTTCTGTGGATGGAGTTATAAAGGAAGTGAATGTGAGCCCATGCCCCACCCA |
| | | | | ACCCTGCCAGCTG AGCAAAGGACAGTCTTACAGCGTCAATGTCACCTTCACCA |
| 10577 | NPC2 | HSX2_02536964 | 555 | GTTCTGTGGATGGAGTTATAAAGGAAGTGAATGTGAGCCCATGCCCCACCCA |
| | | | | ACCCTGCCAGCTG |
| | | | | AGCAAAGGACAGTCTTACAGCGTCAATGTCACCTTCACCAGCA |
| 10577 | NPC2 | HSX2_02536987 | 556 | GTTTCTCATCTCTAAGTGCCTCATTGAGTTCGGTGCATCTGGCCAATGAGTCTG |
| | | | | CTGAGACTCTTG |
| | | | | ACAGCACCTCCAGCTCTGCTGCTTCAACAACAGTGACTTGCTCTCCAATGGTAT |
| | | | | CCAGTGATTCGT |
| | | | | TGAAGAGGAGGTGCTCTGTAGCAGAAACTGAGCTCCGGGTGGCTGGTTCTCA |
| | | | | GTGGTTGTCTCA |
| | | | | TGTCTCTTTTTCTGTCTTAGGTGGTTTCATTAAATGCAGCACTTGGTTAGCAGAT |
| | | | | GTTTAATTTTTTT |
| | | | | TTTAACAACATTAACTTGTGGCCTCTTTCTACACCTGGAAATTTACTCTTGAATA |
| | | | | AATAAAAACTCG TTTGTCTTGTC |
| 56654 | NPDC1 | HSX2_01896007 | 557 | ACCTCGAGGCCCCCGGGGAGGGGCAGGGCCTGGAGCTTCCCACTAAAAACAT |
| | | | | GTTTTGATGCTG |
| | | | | TGTGCTTTTGGCTGGGCCTCGGGCTCCAGGCCCTGGGACCCCTTGCCAGGGAG |
| | | | | ACCCCCGAACC |
| | | | | TTTGTGCCAGGACACCTCCTGGTCCCCTGCACCTCTCCTGTTCGGTTTAGACCC |
| | | | | CCAAACTGGAGG |
| | | | | GGGCATGGAGAACCGTAGAGCGCAGGAACGGGTGGGTAATTCTAGAGACAA |
| | | | | AAGCCAATTAAA GTCCATTTCAGACCTGCGGCTTCTG |
| 1728 | NQO1 | HSX2_00881265 | 558 | GCAGAAGAGCACTGATCGTACTGGCTCACTCAG |
| 1728 | NQO1 | HSX2_02498359 | 559 | AGTAAGAAGGCAGTGCTTTCCATCACCACTGGTGGCAGTGGCTCCATGTACTC |
| | | | | TCTGCAAGGGAT CCACGGGGACATGAATGTCATTCTCTGGCCAATTCAG |
| 4897 | NRCAM | HSX2_01095706 | 560 | GATCACCATTGTGGACCAAAGAAAAACTTGAACCAATCACACTTCAAAGTGGT |
| | | | | CAGTCTTTAGTA |
| | | | | CTTCCCTGCAGACCCCCAATTGGATTACCACCACCTATAATATTTTGGATGGAT |
| | | | | AATT |
| 4897 | NRCAM | HSX2_01095731 | 561 | CGGCTCCATACTGGATCACAGCCCCTCAAAATCTTGTGCTGTCCCCAGGAGAG |
| | | | | GATGGGACCTTG |
| | | | | ATCTGCAGAGCTAATGGCAACCCCAAACCCAGAATTAGCTGGTTAACAAATGG |
| | | | | AGTCCCAATAGA AA |
| 4897 | NRCAM | HSX2_01095756 | 562 | GTTCACTGTTGACAAGGATCATCTAGTGGTAGCTGATGTCAGTGACGATGACA |
| | | | | GCGGGACCTACA |
| | | | | CGTGTGTGGCCAACACCACTCTGGACAGCGTCTCCGCCAGCGCTGTGCTTAGC |
| | | | | GTTGTTG |
| 4897 | NRCAM | HSX2_01095795 | 563 | TCCCCAGTGCTCCCTCGTCTTTGAAGATTGTGAATCCAACACTGGACTCTCTCA |
| | | | | CTTTGGAATGGG |
| | | | | ATCCACCGAGCCACCCGAATGGCATTTTGACAGAGTACACCTTAAAGTATCAG |
| | | | | CCAA |
| 4897 | NRCAM | HSX2_01095798 | 564 | TAATACTCCAGAAGGAGTCCCCAGTGCTCCCTCG |
| 4897 | NRCAM | HSX2_01095800 | 565 | TTAACAGCACACATGAATTAGGCCCTCTGGTAGATTTGAAAATTCCTGCCAACA |
| | | | | AGACACGGTGG |
| | | | | ACTTTAAAAAATTTAAATTTCAGCACTCGATATAAGTTTTATTTCTATGCACAAA |
| | | | | CATCAGCAGGAT |
| | | | | CAGGAAGTCAAATTACAGAGGAAGCAGTAACAACTGTGGATGAAG |
| 4897 | NRCAM | HSX2_01095803 | 566 | CTTAAAGTATCAGCCAATTAACAGCACACATGAA |
| 4897 | NRCAM | HSX2_01095810 | 567 | TTAAAGAAAAGGAAGATGCCCATGCTGACCCTGAAATCCAGCCTATGAAGGA |
| | | | | AGATGATGGGAC ATTTGGAGAATACAG |
| 4897 | NRCAM | HSX2_01095844 | 568 | GGAAGTACCTCTTGATCTGGTACAGCCTCCAACC |
| 4907 | NT5E | HSX2_01096741 | 569 | ATTAACAACAACCTGAGACACACGGATGAAATGTTCTGGAACCACGTATCCAT |
| | | | | GTGCATTTTAAAT |
| | | | | GGAGGTGGTATCCGGTCGCCCATTGATGAACGCAACAATG |
| 4915 | NTRK2 | HSX2_02510945 | 570 | GCGTCAGCCCTCACGTCACTTCGCCAGCAGTAGCAGAGGCGGCGGCGGCGGC |
| | | | | TCCCGGAATTGG |
| | | | | GTTGGAGCAGGAGCCTCGCTGGCTGCTTCGCTCGCGCTCTACGCGCTCAGTCC |
| | | | | CCGGCGGTAGC AGGAGCCTGGACCCAGGC |
| 4926 | NUMA1 | HSX2_02511046 | 571 | CTTTCTCTCAGGTTCTCCAGCTTCTCCCATGGGTGATATCCTGCAGACCCCACA |
| | | | | GTTCCAGATGAG |
| | | | | ACGGCTGAAGAAGCAGCTTGCTGATGAGAGAAGTAATAGGGATGAGCTGGA |
| | | | | GCTGGAGCTAGC TGAGAACCGCAAGCTCCTCACCGAGAAGG |
| 9688 | NUP93 | HSX2_01427660 | 572 | GGATGAGAAAGATAGTCAAGGAGAAAACATGTTTCTGCGCTGTGTGAGTGAG |
| | | | | CTTGTGATTGAA AGCCGAGAG |
| 91775 | NXPE3 | HSX2_02574394 | 573 | AGGAAGCGGGGGGCTAGCGGCCTGGCGCTGCGGCGACCGACCGGGGCGTCA |
| | | | | GGATCCCTGGC CCCCGGAGAGCGAAGGGCGGGCGGGTCCCGGAG |
| 4967 | OGDH | HSX2_01100520 | 574 | CCCTGGGGATTTTGGATGCTGATCTGGACTCCTCCGTGCCCGCTGACATTATCT |
| | | | | CATCCACAGACA AACTT |
| 283208 | P4HA3 | HSX2_02325767 | 575 | TGGCCAACAAGTGGATACATGAGTATGGACAGGAATTCCGCAGACCCTGCAG |
| | | | | CTCCAGCCCTGA |
| | | | | AGACTGAACTGTTGGCAGAGAGAAGCTGGTGGAGTCCTGTGGCTTTCCAGAG |
| | | | | AAGCCAGGAGCC AAAAGCTG |
| 5053 | PAH | HSX2_01105450 | 576 | GAGACTCCATAAAGGCATATGGTGCTGGGCTCCTGTCATCCTTTGGTGAATTA |
| | | | | CAG |
| 5066 | PAM | HSX2_01106278 | 577 | ACAGCCTTTAATTGCTGGCATGTACCTTATGATGTCTGTTGACACTGTTATCCC |
| | | | | AGCAGGAGAAAA AG |
| 5087 | PBX1 | HSX2_01107803 | 578 | GAGCAGAAGAGGAAGAGCCGGGGGCTGCCGTAGCCTTTGGAGATGGACGAG |
| | | | | CAGCCCAGGCT |
| | | | | GATGCATTCCCATGCTGGGGTCGGGATGGCCGGACACCCCGGCCTGTCCCAG |
| | | | | CACTTGCAGGAT GGGGCCGGAGGGACCGAGGGGGAGGGCGGGAG |
| 5087 | PBX1 | HSX2_01107842 | 579 | CCAACGTGCAATCACAGGTGGATACCCTTCGCCA |
| 5090 | PBX3 | HSX2_01107983 | 580 | GAAGCAGGACATCGGCGACATCCTCCACCAGATCATGACCATCACCGACCAGA |
| | | | | GCTTGGACGAG GCGCAAGCAAA |
| 5090 | PBX3 | HSX2_01107986 | 581 | GCGGACGGCGACGGCAGGAAGCAGGACATCGGCG |
| 5090 | PBX3 | HSX2_01107988 | 582 | GAAACATGCCCTGAACTGTCACAGAATGAAACCAGCGCTCTTCAGCGTCCTGT |
| | | | | GTGAGATCAAAG AGAAAACAG |
| 5090 | PBX3 | HSX2_01107991 | 583 | GACGAGGCGCAAGCAAAGAAACATGCCCTGAACT |
| 5090 | PBX3 | HSX2_01107992 | 584 | GTCTCAGCATCAGAGGAGCCCAGGAGGAGGACCCTCCCGATCCCCAGCTAAT |
| | | | | GAGACTGGACAA TATGCTTTTGGCAGAAGGGGTTTCAGGTCCTGAGAAAG |
| 5090 | PBX3 | HSX2_01107994 | 585 | CCGCGGCAGCCTCTGGAGGTTCTTCAGATAACTCTATTGAACACTCAGATTACA |
| | | | | GAGCCAAATTG |
| | | | | ACCCAGATCAGACAAATCTATCACACAGAACTGGAGAAATATGAACAG |
| 5090 | PBX3 | HSX2_01107996 | 586 | GGCGGCAGCAGCTGCAGCCGCGGCAGCCTCTGGA |
| 5090 | PBX3 | HSX2_01107998 | 587 | GATCAAAGAGAAAACAGGTCTCAGCATCAGAGGA |
| 5090 | PBX3 | HSX2_01108000 | 588 | GCATGTAATGAATTTACTACACATGTGATGAACCTTCTCCGAGAACAGAGTAG |
| | | | | AACACGTCCCATT |
| | | | | TCTCCAAAAGAGATTGAAAGAATGGTGGGCATCATCCATCGAAAATTTA |
| 5090 | PBX3 | HSX2_01108001 | 589 | GTTCCATTCAGATGCAGCTCAAACAAAGCACTTGTGAAGCAGTTATGATTTTAA |
| | | | | GATCAAGGTTCC TTGATGCCAG |
| 5090 | PBX3 | HSX2_01108005 | 590 | TGGAGAAATATGAACAGGCATGTAATGAATTTAC |
| 5090 | PBX3 | HSX2_01108007 | 591 | ACGGAAAAGGCGTAACTTCAGTAAACAGGCCACAGAAATCTTGAATGAATATT |
| | | | | TTTACTCACACC |
| | | | | TCAGCAACCCCTACCCCAGTGAAGAAGCCAAAGAGGAGCTGGCCAAGAAATG |
| | | | | CAGCATCACAGT GTCACAG |
| 5090 | PBX3 | HSX2_01108012 | 592 | GTATCCAATTGGTTTGGCAACAAACGAATCAGGTACAAGAAGAACATTGGCA |
| | | | | AGTTTCAGGAAG |
| | | | | AAGCCAACCTCTATGCTGCAAAGACGGCCGTGACAGCTGCACACGCAGTAGC |
| | | | | AGCAGCTGTGCA GAACAACCAGACCAATTCGCCCACCACACCAAA |
| 5090 | PBX3 | HSX2_01108019 | 593 | AGTCGGAGCCAATGTGCAATCACAGGTTCTTCTGGTTCTTTTAACCTCCCAAAT |
| | | | | TCTGGGGACATG |
| | | | | TTCATGAACATGCAGAGTCTGAATGGGGATTCTTACCAAGGGTCCC |
| 5090 | PBX3 | HSX2_01108031 | 594 | GCTAATGGAGGCTGGCAGGACGCAACAACTCCATCTTCTGTGACTTCTCCTAC |
| | | | | AGAAGGCCCAGG |
| | | | | AAGTGTGCACTCGGATACCTCTAACTAATCTCTGGCCACACTTTTCCCTGAGCT |
| | | | | ACATGCCTTGAT |
| | | | | AAGTGCATTCAGAGCAATAGGAGGAAAAGGAAAGCGTTTTTGTAGCCCACCA |
| | | | | TCTACAGCTTTAC |
| | | | | TGTAAAACCTTGTCTTATTCGAGAACTTGGTAAATCTGTTTTTTAAGGAATCAT |
| | | | | AATCATTTGTATT |
| | | | | TATACTTAAAAACACACAATGTTAAAAAAAATAAAGCACTTTATCCAATTAGGC |
| | | | | CAAGATTTAACA |
| | | | | TTGTTGACAGTCCTGTAGCTATTTTATCATAATTTATTATCAATATTTTACATTA |
| | | | | ATGGTTTCACAGT |
| | | | | TGCCAATTACTTGGCCTTAAGGGTAAAAAGTACAATATACACTAAACCTCAACC |
| 5090 | PBX3 | HSX2_01108032 | 595 | GTTAAAGCAGATGCAAAAATTCACCTCACCTAAATTGAACTTCTTGCATATTTC |
| | | | | CATTACTGACTTG |
| | | | | GATTGTCTTTCTTTCATATCACTAATGGAGTTGGAATAAAGAGCTGTTTGCCTA |
| | | | | TCCCTGTTAATGA |
| | | | | TGGTTGTGTTTAAGAATCTTCCTCGTCACGTTTGTGTTCAGATCTCTTATGTTAT |
| | | | | AATTAGATCAGA |
| | | | | GACTGGTAGCATCGTTTCTCTCTCTGAAAGCACCAGTGCCCAGAGTCTGCTCG |
| | | | | GTAATAAAATTAT |
| | | | | GGATCCAGATTGTTCTGAGAGACGAAGATACTTGCTGCTGATAGAGGTGAAA |
| | | | | ACGAGATTGATC |
| | | | | CGTCTGGGGTTTTACGGTGTGCACTGGGTGCTGCACAGACTTGTCAAGGTTTG |
| | | | | CTACGTCCTCTG |
| | | | | GGCATCTGCAAAAGGCCCTGCTCTCTGGAGTGTTGTATATAGTGTAGCAAAAG |
| | | | | AGTATTTATACA |
| | | | | TCCCACCAATCAAAACACAGCTTTATTACCTCATGCGAACTCATACAAACCAAT |
| | | | | AGAATTTCAACA |
| | | | | TGTTCTGTAGCTTAGAGTGCTCACTTACTACCTCTGAACAATACTCACGCTGTA |
| | | | | GTTTGTCTCTTTC |
| | | | | TTATCTTTTTGCATCTTGTAATTAACTCTTTGTTTCCCTTCATAAAATGTAATGTA |
| | | | | CATTGTAATCTTT |
| | | | | TAAAAGAAAAATCAGGGTTGCACTTGCAACTTTTAAAAAACCGAGTGTGGAAA |
| | | | | CATTGGGTCTTA |
| | | | | ATTCAACACAGGATCGGTAAAACTGTTGTAAATACTGAGAAACATTTTGAATG |
| | | | | TTCTTCATCTTATT ACTAATCCATGCAA |
| 5090 | PBX3 | HSX2_02511701 | 596 | CCGCCGCCGCCGCCGCCGCCTCAGCCTTCGCCTCAGCCGCCGCCCGCTCCCGC |
| | | | | CCGCGCGCGGC |
| | | | | GGGATGGACGATCAATCCAGGATGCTGCAGACTCTGGCCGGGGTGAACCTGG |
| | | | | CTGGCCACTCGG |
| | | | | TGCAGGGGGGCATGGCCCTGCCGCCTCCCCCGCACGGCCACGAAGGGGCGG |
| | | | | ACGGCGACGGCA |
| | | | | GGAAGCAGGACATCGGCGACATCCTCCACCAGATCATGACCATCACCGACCA |
| | | | | GAGCTTGGACGA GGCGCAAGCAAA |
| 5090 | PBX3 | HSX2_02511702 | 597 | GTCTCAGCATCAGAGGAGCCCAGGAGGAGGACCCTCCCGATCCCCAGCTAAT |
| | | | | GAGACTGGACAA |
| | | | | TATGCTTTTGGCAGAAGGGGTTTCAGGTCCTGAGAAAGGTGGGGGATCGGCG |
| | | | | GCAGCAGCTGC |
| | | | | AGCCGCGGCAGCCTCTGGAGGTTCTTCAGATAACTCTATTGAACACTCAGATT |
| | | | | ACAGAGCCAAAT |
| | | | | TGACCCAGATCAGACAAATCTATCACACAGAACTGGAGAAATATGAACAG |
| 5090 | PBX3 | HSX2_02511706 | 598 | GCATGTAATGAATTTACTACACATGTGATGAACCTTCTCCGAGAACAGAGTAG |
| | | | | AACACGTCCCATT |
| | | | | TCTCCAAAAGAGATTGAAAGAATGGTGGGCATCATCCATCGAAAATTTAGTTC |
| | | | | CATTCAGATGCA |
| | | | | GCTCAAACAAAGCACTTGTGAAGCAGTTATGATTTTAAGATCAAGGTTCCTTG |
| | | | | ATGCCAG |
| 5090 | PBX3 | HSX2_02511710 | 599 | GTATCCAATTGGTTTGGCAACAAACGAATCAGGTACAAGAAGAACATTGGCA |
| | | | | AGTTTCAGGAAG |
| | | | | AAGCCAACCTCTATGCTGCAAAGACGGCCGTGACAGCTGCACACGCAGTAGC |
| | | | | AGCAGCTGTGCA GAACAACCAGACCAATTCGCCCACCACACCAAATTCCG |
| 5090 | PBX3 | HSX2_02511713 | 600 | GCTAATGGAGGCTGGCAGGACGCAACAACTCCATCTTCTGTGACTTCTCCTAC |
| | | | | AGAAGGCCCAGG |
| | | | | AAGTGTGCACTCGGATACCTCTAACTAATCTCTGGCCACACTTTTCCCTGAGCT |
| | | | | ACATGCCTTGAT |
| | | | | AAGTGCATTCAGAGCAATAGGAGGAAAAGGAAAGCGTTTTTGTAGCCCACCA |
| | | | | TCTACAGCTTTAC |
| | | | | TGTAAAACCTTGTCTTATTCGAGAACTTGGTAAATCTGTTTTTTAAGGAATCAT |
| | | | | AATCATTTGTATT |
| | | | | TATACTTAAAAACACACAATGTTAAAAAAAATAAAGCACTTTATCCAATTAGGC |
| | | | | CAAGATTTAACA |
| | | | | TTGTTGACAGTCCTGTAGCTATTTTATCATAATTTATTATCAATATTTTACATTAA |
| | | | | TGGTTTCACAGT |
| | | | | TGCCAATTACTTGGCCTTAAGGGTAAAAAGTACAATATACACTAAACCTCAACC |
| | | | | GTTAAAGCAGAT |
| | | | | GCAAAAATTCACCTCACCTAAATTGAACTTCTTGCATATTTCCATTACTGACTTG |
| | | | | GATTGTCTTTCTT |
| | | | | TCATATCACTAATGGAGTTGGAATAAAGAGCTGTTTGCCTATCCCTGTTAATGA |
| | | | | TGGTTGTGTTTA |
| | | | | AGAATCTTCCTCGTCACGTTTGTGTTCAGATCTCTTATGTTATAATTAGATCAGA |
| | | | | GACTGGTAGCA |
| | | | | TCGTTTCTCTCTCTGAAAGCACCAGTGCCCAGAGTCTGCTCGGTAATAAAATTA |
| | | | | TGGATCCAGATT |
| | | | | GTTCTGAGAGACGAAGATACTTGCTGCTGATAGAGGTGAAAACGAGATTGAT |
| | | | | CCGTCTGGGGTT |
| | | | | TTACGGTGTGCACTGGGTGCTGCACAGACTTGTCAAGGTTTGCTACGTCCTCT |
| | | | | GGGCATCTGCAA |
| | | | | AAGGCCCTGCTCTCTGGAGTGTTGTATATAGTGTAGCAAAAGAGTATTTATAC |
| | | | | ATCCCACCAATCA |
| | | | | AAACACAGCTTTATTACCTCATGCGAACTCATACAAACCAATAGAATTTCAACA |
| | | | | TGTTCTGTAGCTT |
| | | | | AGAGTGCTCACTTACTACCTCTGAACAATACTCACGCTGTAGTTTGTCTCTTTCT |
| | | | | TATCTTTTTGCAT |
| | | | | CTTGTAATTAACTCTTTGTTTCCCTTCATAAAATGTAATGTACATTGTAATCTTTT |
| | | | | AAAAGAAAAATC |
| | | | | AGGGTTGCACTTGCAACTTTTAAAAAACCGAGTGTGGAAACATTGGGTCTTAA |
| | | | | TTCAACACAGGA |
| | | | | TCGGTAAAACTGTTGTAAATACTGAGAAACATTTTGAATGTTCTTCATCTTATTA |
| | | | | CTAATCCATGCA A |
| 80714 | PBX4 | HSX2_02053487 | 601 | CATGGCCGCCCCGCCGCGCCCCGCGCCATCGCCCCCCGCCCCGCGGCGCCTCG |
| | | | | ACACGAGCGAC |
| | | | | GTCCTGCAGCAGATCATGGCCATCACCGACCAGAGCCTGGACGAGGCACAGG |
| | | | | CCAG |
| 80003 | PCNXL2 | HSX2_02036459 | 602 | CATGTGAGTCAGGATCTGGAAGCCTCGTCATGTTCTTCAACACAAGGAAAATT |
| | | | | TAACCGAGAGCA |
| | | | | GTTTTACAAATTTATCATTTTCCCTGGCAAGTGGATTAAAGTCTGGTATGATCG |
| | | | | ACTGACCTTGCT GGCATTACTTGATCG |
| 80003 | PCNXL2 | HSX2_02036493 | 603 | GGGCCAAAGCCAGGCACCCTCCCAGTTACGTTGTGTATGGCCTGAAGCTCTTC |
| | | | | TCTCCAGTGTTTC TACAATCAGCTAGGGACTACTTAATAG |
| 80003 | PCNXL2 | HSX2_02036518 | 604 | GAACCGTGGAGCATGCAACACATCCCGGCACTGTTTTCGGCCTTCTGTGGCCT |
| | | | | CTTGGTCGCCCTT |
| | | | | TCTTACCATCTGAGCCGTCAGAGCAGTGACCCATCTGTACTCAT |
| 80003 | PCNXL2 | HSX2_02036531 | 605 | ACGGATGTCTTAAAATGGGATCTCATCGTCTGCGCAGTGGTTGCTGTCCTCTCA |
| | | | | TTTGCAGTCAGC GCCAGCACTGTATTCCTGTCATTGCGA |
| 80003 | PCNXL2 | HSX2_02036542 | 606 | ATGTTGCCCATTTAATGTGGTTCGAAAGACTCTATGTTTGGCTTCAGTGTTTTG |
| | | | | AAAAATACATCTT |
| | | | | GTACCCAGCGCTAATTTTGAATGCCCTCACTATTGATGCATTTTTAATAAGCAA |
| | | | | TCACCGGAGACT TGGTACCCA |
| 80003 | PCNXL2 | HSX2_02036681 | 607 | CACCTCTCCGAGCCATGTGAGCCCCCTGATGCCACCGAGCAGGGGCAGCTTCA |
| | | | | TGACCGATGTCT |
| | | | | GGCTGAGGCTGTGGCGGACACTCTCGGGGTTGTCTGCAGGAGAGCAAGCCAG |
| | | | | GAGGACATGGG |
| | | | | CCTGGACGACACGGCCTCGCAGCAAAGTGTGTCAGACGAGCAGTGACGGGCG |
| | | | | TGCGGCCGGGC |
| | | | | GGGGAGGCTGGCTCCCCCACACCTCCCACCTGCATTGCTCTCCCTCGTGCTCCC |
| | | | | CAAATCACCACA |
| | | | | ACCAACCAATACCGCGATCCATGAGGGACTCCTCCTGTGGAAAAGGAGAGCT |
| | | | | GTTCCAGAACAC AGAACTGATCTCAGGTTTTTG |
| 27344 | PCSK1N | HSX2_01710668 | 608 | GTACTTGCTGGGACGGATTCTTGCGGGAAGCGCGGACTCCGAGGGGGTGGCA |
| | | | | GCCCCGCGCCG |
| | | | | CCTCCGCCGTGCCGCCGACCACGATGTGGGCTCTGAGCTGCCCCCTGAGGGCG |
| | | | | TGCTGGGGGCG |
| | | | | CTGCTGCGTGTGAAACGCCTAGAGACCCCGGCGCCCCAGGTGCCTGCACGCC |
| | | | | GCCTCTTGCCACC |
| | | | | CTGAGCACTGCCCGGATCCCGTGCACCCTGGGACCCAGAAGTGCCCCCGCCAT |
| | | | | CCCGCCACCAG |
| | | | | GACTGCTCCCCGCCAGCACGTCCAGAGCAACTTACCCCGGCCAGCCAGCCCTC |
| | | | | TCACCCGAGGAT CCCTACCCCCTGGCCCCACAATAAACATGATCTGAAGCA |
| 5125 | PCSK5 | HSX2_01110707 | 609 | GGGCAGGAGCTGATGGGTGCATTAACTGCACAGAGGGCTACTTCATGGAGGA |
| | | | | TGGGAGATGCG |
| | | | | TGCAGAGCTGTAGTATCAGCTATTACTTTGACCACTCTTCAGAGAATGGATACA |
| | | | | AATCCTGCAAAA A |
| 8654 | PDE5A | HSX2_01350859 | 610 | CGAGGCCGAGTCCTGTTCTTCTGAGGGACGGACCCCAGCTGGGGTGGAAAAG |
| | | | | CAGTACCAGAG |
| | | | | AGCCTCCGAGGCGCGCGGTGCCAACCATGGAGCGGGCCGGCCCCAGCTTCGG |
| | | | | GCAGCAGCGAC |
| | | | | AGCAGCAGCAGCCCCAGCAGCAGAAGCAGCAGCAGAGGGATCAGGACTCGG |
| | | | | TCGAAGCATGGC |
| | | | | TGGACGATCACTGGGACTTTACCTTCTCATACTTTGTTAGAAAAGCCACCAG |
| 8654 | PDE5A | HSX2_01350876 | 611 | GACTTTGCTGCTTATTTGGCATTTTGTGGTATTGTTCTTCATAATGCTCAGCTCT |
| | | | | ATGAGACTTCAC TGCTGGAGAACAAGAGAAATCAG |
| 8654 | PDE5A | HSX2_01350881 | 612 | GTGCTGCTTGACCTTGCTAGTTTAATTTTTGAAGAACAACAATCATTAGAAGTA |
| | | | | ATTTTGAAGAAA |
| | | | | ATAGCTGCCACTATTATCTCTTTCATGCAAGTGCAGAAATGCACCATTTTCATA |
| | | | | GTGGATGAAGAT TGCTCC |
| 8654 | PDE5A | HSX2_01350921 | 613 | AACAAGCTGACTGACCTGGAGATACTTGCATTGCTGATTGCTGCACTAAGCCA |
| | | | | CGATTTGGATCA CCGTGGTGTGAATAACTCTTACATACAGCG |
| 8654 | PDE5A | HSX2_01350941 | 614 | GGCAATGCTGATGACAGCTTGTGATCTTTCTGCAATTACAAAACCCTGGCCTAT |
| | | | | TCAACAACGG |
| 8654 | PDE5A | HSX2_01350956 | 615 | GCCCTGACCCACGTGTCAGAGGACTGTTTCCCTTTGCTAGATGGCT GCAGAAA |
| | | | | GAACAGGCAGA |
| | | | | AATGGCAGGCCCTTGCAGAACAGCAGGAGAAGATGCTGATTAATGGGGAAAG |
| | | | | CGGCCAGGCCA |
| | | | | AGCGGAACTGAGTGGCCTATTTCATGCAGAGTTGAAGTTTACAGAGATGGTGT |
| | | | | GTTCTGCAATAT |
| | | | | GCCTAGTTTCTTACACACTGTCTGTATAGTGTCTGTATTTGGTATATACTTTGCC |
| | | | | ACTGCTGTATTTT |
| | | | | TATTTTTGCACAACTTTTGAGAGTATAGCATGAATGTTTTTAGAGGACTATTAC |
| | | | | ATATTTTTTGTAT |
| | | | | ATTTGTTTTATGCTACTGAACTGAAAGGATCAACAACATCCACTGTTAGCACAT |
| | | | | TGATAAAAGCAT |
| | | | | TGTTTGTGATATTTCGTGTACTGCAAAGTGTATGCAGTATTCTTGCACTGAGGT |
| | | | | TTTTTTGCTTGGG |
| | | | | GATTATTTTAAATAATTGGTTTTTGTGTTTTCTGAATTACCATTTTTTCAAGAAT |
| | | | | GTTTGGAATCTTT |
| | | | | CCTTTTTCAAAAGTAGGTTAGGAGCAAATTATCATACATTCTGTGACATTTAAA |
| | | | | GCCTTTATAGGA |
| | | | | TAGTGAAAAATGCTGGCTGAGTGGATTTTAAGAGAAATAATTGTATTTGTTAA |
| | | | | CAGTGTCTTTTTT |
| | | | | TAAAAAGTTAAGGCACTCTGAAACAAATGGAAAGTCCTATGAAACTGTATTGT |
| | | | | AAAGAAAACATT |
| | | | | ATTTAATTGATATGCTGTTTTGTGAGAGAACAGGCAAGACAGAACTTTGTCACT |
| | | | | TCAGTGCAGTAC |
| | | | | ATTTTTCTGAAAGCTACCCATAAAATCACTTTCATCTCACCTACCTGATGCAAAG |
| | | | | CAGGTGAAACC |
| | | | | TTAGGAGATGATCCAGTCACTGACTTGATTGAGGGATAAGTGTGATTTAGAAA |
| | | | | TGGAATGGCCTT |
| | | | | GGATGTCTATCAGTGAAGAAAAATGTTCTGTTAGAAGATCTCTCTAAGAGTTTT |
| | | | | TTTCCTTCTGAG |
| | | | | CTTCCTTTTCAAAATAAAAGTGACAATTGTAGCATTGACTTGAAGTGAGACATG |
| | | | | GTTATAGATAAG |
| | | | | AGAGTACAAAATGACTCTTTTTCCTGTCAATTGAAATTTAAAGAAAAGTTTTAA |
| | | | | TTATATAAATAGC |
| | | | | AAAGGGCTATTGCCAATACTAGGGTCAAAAATGAATTTGAGGGAACAGTGGG |
| | | | | TAAGAAACTTTA |
| | | | | TGCCTGAATAACATTTAGCAGTATTGTGATTGAAAAATTGCCATATTTTGATGT |
| | | | | ATAGGACAAGTC |
| | | | | AACTGAGATCCAGAGAATCCTGGATGTGAATGCTAAACACTGGCCCTTAACTC |
| | | | | ACATTCAATGTAT |
| | | | | TTTCTTCCCATAACATTTAGTATAGTTAATATTTTCTTAGAATTTGAGCCCATTTA |
| | | | | AGTGGATTAATA TTCTACATGTGTGCCCCTAAAGACACATTT |
| 8654 | PDE5A | HSX2_01350958 | 616 | CTTCTGCTCTACTACAGAGAGTGAAGAAGAAAGTATTAAAGCTCACTTTACCAT |
| | | | | TCCATATACTTA |
| | | | | CTAAAAGCCTGTGTAAACATGTCTTAATGAATGTTGTTGAAAGCAATGTAAAT |
| | | | | AGTTGAAAATATA |
| | | | | AATTTATATTACAGTTTAAGAAAACCTTATGAGGCATCACTAGCCACTGTTAAT |
| | | | | ATCTATTTGTATT |
| | | | | CTTATACCTTTTCAATATATTTGAACAAATATAGTTTCTGGCACTATTTTTATACT |
| | | | | AGGAAAAGGAG |
| | | | | TTACTATGTATATTATGCTTAGCTTTTAAGGCATTTTAAATAACCATGAATGTTG |
| | | | | ATTTCATTACTTT |
| | | | | CCTTTCCTCCATCACGAGAGTCATTTCAGATGACTCTTTCATGACAAAATCACTT |
| | | | | TAAAGGAACACT |
| | | | | TACCTCCGATTCCTGTATAAAGTCATGAGATGGTCAAGGTGGTTTTCCATTGTG |
| | | | | CAAATTCTTCAC |
| | | | | CTGTCAGTGGTTTCCTCATTTTGCCATGCTTTGTAAAAATAAAAAGAATGATCA |
| | | | | AGTAGGTATGAA |
| 5162 | PDHB | HSX2_02512202 | 617 | AAGGTGGTCAGTCCCTGGTGTAGCTGCCCAGCAC |
| 5164 | PDK2 | HSX2_02512156 | 618 | GAGCGCTGCCCGCGCGGGGACCACAACCAAAGTCGCGGCCGCCGCAGCCATG |
| | | | | CGCTGGGTGTG |
| | | | | GGCGCTGCTGAAGAATGCGTCCCTGGCAGGGGCGCCCAAGTACATAGAGCAC |
| | | | | TTCAGCAAGTTC TCCCCGTCCCCGCTGTCCATGAAGCAGTTTCTGGACTTCG |
| 5166 | PDK4 | HSX2_01115087 | 619 | GTATATACAGAGCCTGATGGATTTGGTGGAATTCCATGAGAAAAGCCCAGATG |
| | | | | ACCAGAAAGCA TTATCAGA |
| 5166 | PDK4 | HSX2_01115092 | 620 | CTTTGTAGATACACTCATCAAAGTTCGAAATAGACACCATAATGTAGTCCCTAC |
| | | | | AATGGCACAAG |
| | | | | GAATCATAGAGTATAAAGATGCCTGTACAGTTGACCCAGTCACCAATCAAAAT |
| | | | | CTTCAATATTTCT |
| | | | | TGGATCGATTTTACATGAACCGTATTTCTACTCGGATGCTGATGAACCAGCACA |
| 5166 | PDK4 | HSX2_01115102 | 621 | ATGCCTTTGAGTGTTCAAGGATGCTCTGTGATCAGTATTATTTATCATCTCCAG |
| | | | | AATTAAAGCTTAC ACAAGTGAATG |
| | | | | AATGCAATGCGGGCAACAGTTGAACACCAGGAAAATCAGCCTTCCCTTACACC |
| 5166 | PDK4 | HSX2_01115112 | 622 | AATAGAGGTTAT TGTTGTCTTGGGAAAAGAAGACCTTACCATTAAG |
| 5166 | PDK4 | HSX2_01115117 | 623 | ATTTCAGACAGAGGAGGTGGTGTTCCCCTGAGAATTATTGACCGCCTCTTTAGT |
| | | | | TATACATACTCC |
| | | | | ACTGCACCAACGCCTGTGATGGATAATTCCCGGAATGCTCCTTTG |
| 5166 | PDK4 | HSX2_01115122 | 624 | GCTGGTTTTGGTTACGGCTTGCCAATTTCTCGTCTGTATGCAAAGTACTTTCAA |
| | | | | GGAGATCTGAAT |
| | | | | CTCTACTCTTTATCAGGATATGGAACAGATGCTATCATCTACTTAAAG |
| 5166 | PDK4 | HSX2_01115127 | 625 | TGAGCTCTGAGGCTGATGACTGGTGTATCCCAAGCAGGGAACCAAAGAACCT |
| | | | | GGCAAAAGAAGT |
| | | | | GGCCATGTGAAGAGGGACACTCAGGACACTTTACGGGATCAAAGTGGGTCTA |
| | | | | CACCAGTGCTGC |
| | | | | TTCCTGAATGTTTGTGTGTGAACCCTTGTTTCCTCCAAAACAAACGACAGCAAC |
| | | | | GAAAACTCCTTA |
| | | | | ATCAGAACACTGATCCAATGAGGAATGGAGCTTGTTTCTGTGACCCAGGAGAA |
| | | | | CTTAGTGCAAGA |
| | | | | CTACAGGAGTTAACAGATGGCCAGCTCCTTATTTTTTAATGTAGAATAACTCCT |
| | | | | GAGTTTATATCA |
| | | | | AATCCTGAAGAAATAAGCCTCAGTTTTCCATCTGTTTTTGATAAGAATAAGAAA |
| | | | | GGGAGTGAGTG |
| | | | | TGAAGATGGTGGTTAGCAGTTTCACTAAGACTGATATTTTAGGCCTCTTGTTCA |
| | | | | CATCAAAAGATA |
| | | | | TTGGTGTCAGAATACCAGCATTTTCCTGCCATGCAAAGGATTAAAACTTAGTTT |
| | | | | ACACTATGTGGT |
| | | | | TACAAATATATGTCAATGTACATTTTGAACATATTTATGTGCTATGGAAGGAAA |
| | | | | TGCTGGTGACTA |
| | | | | AAATAAGGTTTACTCTGAAAGAGGAGGAATTTTATTCAAAGCATTCAAACATTT |
| | | | | TATTCAAGTGTT |
| | | | | TCAAAATTCAAAGCATTGTATTCAAAGTTGCAGTGAAGGCATCAACTTATGTAA |
| | | | | AAACTCAGAAG |
| | | | | GAAGGCTCCTCTGATAAAAACACAGCTCCTTTATTATGCTGCTTTTCTTGTTCAC |
| | | | | TTTACACACTAA |
| | | | | GTAAACACTTATTGTCAGGTGCCTAGTCTTGAGTGAATTGTTAGATGTGCACTG |
| | | | | AACTCGGGATG |
| | | | | TTGGGGATTGGAGAGAGAGAATTGCCAAAGTAACAGCAAAAATATCTCTTACT |
| | | | | TTGCTTTGTTTAT |
| | | | | AAATAAATTAGTAGATTGGAAAAACTAGTGTTAGGGAAAGAAATCACATGTTC |
| | | | | AGAGCCTAATTC |
| | | | | AGTAGGAAGGGCTTTTCTCTACCCTGAAATGAAGGTAATCCAAAGGCATCCAT |
| | | | | TTTCTAGGCTTAA |
| | | | | AAGATATATTTTTGATATATTTAATTATATTCTCTACACTCCAGCATTAATATGT |
| | | | | CTGTTTAAAAATT |
| | | | | ACTAATTCTCAAATGGCTCAAGAACATTAGAATTTAAGTACCTTTTAGAGTAAT |
| | | | | TATTTTAAGCAAA |
| | | | | TAGCCTGGACGTAAGAGATTCTCATGCCAGCATGCTTTCATTTGTCAGTTGTTG |
| | | | | TGACTGAGAGAT |
| | | | | AATGAATGACACCTGAAATGCATATGGTATTTTTGGGAGAGTTAAGGTATAAT |
| | | | | TTGAAGGTTGGC |
| | | | | AGACCAGTTGCGCTGATTACTCTTAGAGAAGAAGAAATGGAAAAATGAAAGA |
| | | | | AGGCAGGAAGG |
| | | | | AAAGAAAGGATATAGGAAGAGAGGGAAGCAGAAGGCAGGCATTTTTCTATTT |
| | | | | TCCCCACAAATT |
| | | | | ATTTCAAAAAAAATCTGTATTTTCTGGGATATGTCATTGGCAAGAGGAAGAAC |
| | | | | TGGTGTTTTGAAA |
| | | | | GCAGTATGGATTCTTTAAATGCCTCTCACTCTTACAAGATAGTAGGCTTTGAGA |
| | | | | TAATAAACTTAC |
| | | | | CCGTGTCAATTAACATTTAAACTGGCATATAGAAAAAAAGGAGGATTTTTCTG |
| | | | | CATTGTAAAATAA |
| | | | | TCAGTATGGTTTATATGTTGAATTTGACATTTGTGTGTAATTTCATGGTGGCCT |
| | | | | AGTGTTGTGGTG |
| | | | | CTTCTGGTAATGGTAATAGAAGCTCAACTATTTTTTTGTGGATTTCAGTTTTTAT |
| | | | | CATCAGAAGTCC |
| | | | | TAGACAGTGACATTTCTTAATGGTGGGAGTCCAGCTCATGCATTTCTGATTATA |
| | | | | CAAAACAGTTTG |
| | | | | CAGTAGGTTATTTGTCATTTCAGTTTTTTACTGAAATTTGAGCTAAACATTTTTA |
| | | | | CATGTAAATACTT |
| | | | | GTATTTACCAAAGATTTAAATCAGTTGATTAATTAATTAACTCAAATACTGTGA |
| | | | | ACTATCTCTAAAA |
| | | | | CACTAGAAAAAAGAAATGTTAGTATCTCAATTACACCAACTGTGCAAATGAAC |
| | | | | TTTGATAAAATAG |
| | | | | AAATAATCTACATTGGCCTTTGTGAAATCTGGGGAAGAGCTTTAGGATTCTAG |
| | | | | TAGATGGATACT |
| | | | | GAATACTCAGGCCCACTTAAATTATTAATGTATACATTGTGTTTTTGTCTTTATG |
| | | | | CTATGTACAGAG |
| | | | | AAATGTGATAATTTTTTATAATAAATATTTTTTATGATGATAA |
| 5255 | PHKA1 | HSX2_01122215 | 626 | GAAATAATGGTATATCTAGCCATGTATATGCGAACCCAGCCTGGCCTCTTTGCT |
| | | | | GAAATGTTTCGA |
| | | | | CTTCGAATTGGTCTGATCATACAAGTTATGGCAACAGAACTGGCCCACTCCCTT |
| | | | | CGATGCTCAG |
| 7262 | PHLDA2 | HSX2_01281663 | 627 | ACGCTGGACGAGTCGGACCGAGGCTAGGACGTGGCCGGCGCTCTCCAGCCCT |
| | | | | GCAGCAGAAGA |
| | | | | ACTTCCCGTGCGCGCGGATCCTCGCTCCGTTGCACGGGCGCCTTAAGTTATTG |
| | | | | GACTATCTAATAT |
| | | | | CTATGTATTTATTTCGCTGGTTCTTTGTAGTCACATATTTTATAGTCTTAATATCT |
| | | | | TGTTTTTGCATC |
| | | | | ACTGTGCCCATTGCAAATAAATCACTTGGCCAGTTTGCTTTTCTACCA |
| 7262 | PHLDA2 | HSX2_02522700 | 628 | GGCCATACGCTGGACGAGTCGGACCGAGGCTAGGACGTGGCCGGCGCTCTCC |
| | | | | AGCCCTGCAGC |
| | | | | AGAAGAACTTCCCGTGCGCGCGGATCCTCGCTCCGTTGCACGGGCGCCTTAAG |
| | | | | TTATTGGACTAT |
| | | | | CTAATATCTATGTATTTATTTCGCTGGTTCTTTGTAGTCACATATTTTATAGTCTT |
| | | | | AATATCTTGTTTT |
| | | | | TGCATCACTGTGCCCATTGCAAATAAATCACTTGGCCAGTTTGCTTTTCTACCA |
| 55650 | PIGV | HSX2_02557993 | 629 | GGAGGAGCTGAGGTTCGGTTCGGTCCCCGCCGGG |
| 93035 | PKHD1L1 | HSX2_02151899 | 630 | AGTCCCAGGAGCCGAGCTCCAGCACTAGAGCCAGCTGCGAGCGGAGGGCACC |
| | | | | AACTCCGCAGA |
| | | | | ACTGGCTTTTCAATGGGACACCTGTGGCTCCTGGGTATTTGGGGCCTCTGTGG |
| | | | | GCTGCTCCTGTGTGCCGCGGATCCCAGCACAG |
| 93035 | PKHD1L1 | HSX2_02151902 | 631 | ATGGCTCTCAAATAATCCCCAAAGTCACAGAAATAATACCTAAATATGGCAGT |
| | | | | ATAAATGGAGCA ACAAGGCTGACTATAAGAGGGGAAG |
| 93035 | PKHD1L1 | HSX2_02151911 | 632 | AGCAATGCCGGAAGATTCCTACACTGTTAGAGTCAGTGTGGACGGGGTTCCTG |
| | | | | TTACGGAAAATA |
| | | | | ACACCTGCAAAGGTCACATCAACAGCTGGGAATGTACCTTCAAC |
| 93035 | PKHD1L1 | HSX2_02151919 | 633 | GCAAAAAGTTTTAGAACCCCAACAATAAGAAGCATCACACCTTTATCTGGAACT |
| | | | | CCAG |
| 93035 | PKHD1L1 | HSX2_02151939 | 634 | AGTTTACATTGGAGGAATGCCCTGTGAGCTTCTCATACCACAATCTGATAATTT |
| 93035 | PKHD1L1 | HSX2_02151983 | 635 | AGGTCACCATGATTTTCCCTTCACAAGGAAGCATTCGAGGTGGCACCACGCTG |
| | | | | ACAATAAGTGGG |
| | | | | CGTTTCTTTGATCAGACAGATTTCCCCGTCAGAGTTCTAGTTGGAG |
| 93035 | PKHD1L1 | HSX2_02151994 | 636 | GTGAACCTTGTGATATTTTGAATGTCACAGAAAATAGTATATGTTGCAAGACA |
| | | | | CCCCCCAAACCTC ATATTCTCAAAACTGTATATCCAG |
| 93035 | PKHD1L1 | HSX2_02152006 | 637 | GAGGGAGAGGCCTGAAGCTTGAGGTGTGGAATAATAGCCGTCCAATACGTTT |
| | | | | GGAAGAGATACT |
| | | | | GGAATACAATGAAAAAACGCCTGGGTACATGGGTGCCAGTTGGGTAGATTCA |
| | | | | GCTTCCTATATTT |
| | | | | GGCTCATGGAACAAGACACATTTGTTGCACGCTTTAGTGGATTTTTGGTGGCTC |
| | | | | CAGATTCTGATG |
| | | | | TTTATAGATTCTACATCAAGGGTGATGACCGTTATGCTATTTATTTTAGCCAGA |
| | | | | CTGGACTTCCAGAAGATAAG |
| 93035 | PKHD1L1 | HSX2_02152050 | 638 | GTTATAACATTGGAAAACTGGGAAACAACTAATGCAATTAATGAGGTTCAGAA |
| | | | | GATCAAGGTAAC |
| | | | | CAGCCCATGTGTGGAAGCTAATTCATGTTCACTTTACCAATATAGATTAATCTA |
| | | | | TAATATGGAAAA AACTG |
| 93035 | PKHD1L1 | HSX2_02152077 | 639 | GAGACTTTGATCTGCTTGGTTATGAAGTAGTTGAAGGGAATAATGTCACACTG |
| | | | | GATATTACAGAA |
| | | | | CAAACCAAAGGAAAACCCAACTTGGAGACATTCACACTGAATTGGGATGGGA |
| | | | | TCGCTTCTAAGCC ACTCACTCTATGGTCATCAGAAGCTGAA |
| 93035 | PKHD1L1 | HSX2_02152101 | 640 | GAACATATTAACAGAGGGCAGAAGACAGCTGAAACCGATGCTTACTGTGGTC |
| | | | | GTTATTCCCTGAA |
| | | | | AAACCCAGCTGTTCTTTTTGACTCAGCAGATGTTAAACCAAACAGACGACCATA |
| | | | | TGGAGATATTTT ATTGTTTCCTTATAATCAG |
| 93035 | PKHD1L1 | HSX2_02152113 | 641 | TTATGTTTAGCATACAAAGGATTCCTGGCAAATTATATTGGTCTAAAATTTCAG |
| | | | | TACCAAGACAAT |
| | | | | AGCAAGATTACTAGAAGCACTGATACACAGTTTACATACAACTTTGCTTATGGA |
| | | | | AACAA |
| 93035 | PKHD1L1 | HSX2_02152180 | 642 | AAAACTGAGGGTACAGTTGATATTTCAGTTACTACCAATGGATTTCAAGCCAC |
| | | | | AGCAAGGGATGC |
| | | | | TTTTAGTTATAATTGTTTACAGACACCAATTATAACTGATTTTAGTCCAAAAGTA |
| | | | | CGAACAATACTA G |
| 93035 | PKHD1L1 | HSX2_02152186 | 643 | GAGAAGTTAATTTAACAATTAAGGGCTATAATTTTGGAAATGAACTCACACAA |
| | | | | AACATGGCGGTG |
| | | | | TATGTTGGAGGAAAAACCTGCCAGATTCTTCACTGGAACTTCACAGATATTAG |
| | | | | ATGCCTTTTGCCC |
| | | | | AAGTTGTCTCCTGGAAAACATGATATCTATGTAGAAGTCAGAAACTGGGGTTT |
| | | | | TGCATCAACAAG |
| 93035 | PKHD1L1 | HSX2_02152208 | 644 | GTTCATTTTCTTACCAATTTACTTCTCCTGGAATCCATTATTATAGCAGCGGGTA |
| | | | | TGTTGATGAGGC |
| | | | | TCACTCCATTTTTCTCCAAGGAGTCATTAATGTTTTACCAGCTGAAACCAGACA |
| | | | | CATTCCCTTGCAC CTGTTTGTGGGTCGCTCTGAAGCCACATATGCTTATG |
| 93035 | PKHD1L1 | HSX2_02152220 | 645 | GTTACAATTGGTAGCTACCCCTGTGTCGTAGAAGAAAGTAGTGAGGATTCAAT |
| | | | | TACATGTCATATT |
| | | | | GACCCTCAAAACTCAATGGATGTTGGTATCAGGGAAACTGTCACTTTGACTGT |
| | | | | CTACAACCTGGG |
| | | | | CACTGCTATCAATACGTTGTCCAATGAATTTGATAGGCGATTTGTACTTTTGCC |
| | | | | AAACATTGACCT |
| | | | | GGTGTTGCCAAATGCAGGATCAACTACAGGAATGACAAGCGTGACCATAAAA |
| | | | | GGCTCTGGATTT |
| | | | | GCCGTTTCTTCTGCAGGTGTAAAAGTCCTTATGGGTCATTTCCCATGTAAAGTT |
| | | | | CTATCAGTGAAT |
| | | | | TATACGGCCATTGAATGTGAAACATCCCCTGCTGCCCAACAGCTTGTGGATGT |
| | | | | AGATCTTCTAATA |
| | | | | CATGGAGTGCCTGCCCAGTGCCAGGGAAACTGCACCTTTTCATACTTAGAAAG |
| | | | | CATCACTCCTTAC |
| | | | | ATAACAGGAGTCTTCCCAAACTCTGTCATAGGATCTGTAAAAGTTCTTATTGAA |
| | | | | GGAGAAGGTTT |
| | | | | GGGGACTGTTTTGGAGGACATTGCTGTTTTCATTGGAAATCAACAGTTCAGAG |
| | | | | CAATAGAGGTTA |
| | | | | ATGAAAACAACATCACTGCTCTTGTGACTCCTCTCCCAGTTGGACATCATTCTG |
| | | | | TTAGTGTTGTGG |
| | | | | TGGGAAGTAAAGGCTTGGCTCTGGGAAACCTGACTGTCAGCAGCCCCCCAGT |
| | | | | AGCATCTCTATCA |
| | | | | CCAACTTCTGGAAGCATTGGTGGTGGAACTACACTGGTGATCACAGGAAATG |
| | | | | GCTTCTATCCAGG |
| | | | | CAACACTACAGTCACTATTGGGGATGAACCTTGTCAAATTATTTCCATCAACCC |
| | | | | CAATGAAGTCTA |
| | | | | CTGCCGCACTCCCGCTGGGACCACTGGAATGGTCGATGTTAAAATCTTTGTTA |
| | | | | ATACAATTGCTTA |
| | | | | TCCACCTTTGCTTTTTACATATGCCCTGGAGGATACTCCATTTCTCAGAGGAAT |
| | | | | TATCCCAAGCAGAG |
| 93035 | PKHD1L1 | HSX2_02152254 | 646 | TGAAAATATGGAGGATGTTCATATCACCATAGCTGAAGCCAAATGTGATGTTG |
| | | | | AGTATTCCAACA |
| | | | | AGACACACATCATCTGCATGACAGATGCCCATACTCTATCAGGGTGGGCTCCA |
| | | | | GTTTGTGTCCAC ATCAGAGGTGTCGGCATGGCCAAACTG |
| 93035 | PKHD1L1 | HSX2_02152259 | 647 | GATAATGCTGACTTTCTTTATGTTGATGCCTGGTCCTCCAATTTCTCATGGGGG |
| | | | | GGAAAATCTCCC |
| | | | | CCAGAAGAAGGATCTCTTGTTGTTATTACAAAAGGACAGACCATTCTGCTGGA |
| | | | | TCAAAGCACCCC TATTTTGAAAATGTTGCTTATTCAGG |
| 93035 | PKHD1L1 | HSX2_02152274 | 648 | ATTGGAACAGAGACATCCCCATTCCAACACAAGGCTGTCATTACCTTGCATGGT |
| | | | | CACCTGCGATCT |
| | | | | CCTGAGCTCCCTGTCTATGGTGCCAAAACACTGGCTGTGCGGGAGGGAATCCT |
| | | | | GGATCTGCACG |
| 93035 | PKHD1L1 | HSX2_02152281 | 649 | GTGTGCCTGTTCCTGTGACCTGGACTCGCTTGGCTCATACTGCAAAGGCAGGG |
| | | | | GAAAGAATTTTA |
| | | | | ATTTTACAAGAAGCAGTAACATGGAAACCAGGAGATAACATTGTAATTGCAAG |
| | | | | CACAGGACACA G |
| 93035 | PKHD1L1 | HSX2_02152287 | 650 | ACACAGTCAAGGAGAGAATGAAAAAATGACCATTGCATCTGTGTCTGCTGATG |
| | | | | GCATAAACATAA |
| | | | | CACTAAGTAACCCACTAAATTACACACACTTAGGAATTACGGTCACACTCCCTG |
| | | | | ATGGAACTCTGT |
| | | | | TTGAAGCAAGAGCAGAAGTTGGAATTCTTACAAGAAATATTTTAATAAGAGGA |
| | | | | TCTGATAATGTT |
| | | | | GAGTGGAATAACAAAATTCCTGCATGTCCTGATGGATTTGACACAG |
| 93035 | PKHD1L1 | HSX2_02152293 | 651 | GAGAATTTGCTACACAGACCTGTCTCCAAGGAAAGTTTGGAGAAGAAATAGG |
| | | | | AAGTGACCAATTT |
| | | | | GGAGGCTGCGTTATGTTTCATGCTCCTGTACCTGGTGCTAACATGGTAACTGG |
| | | | | GAGAATAGAATA TGTAGAG |
| 93035 | PKHD1L1 | HSX2_02152298 | 652 | GTATTCCATGCTGGCCAGGCTTTCCGGTTGGGGCGATATCCAATACATTGGCA |
| | | | | CCTGCTTGGAGA |
| | | | | CTTACAGTTTAAATCTTATGTAAGAGGCTGTGCAATTCACCAGGCCTATAACAG |
| | | | | AGCTGTTACTAT |
| | | | | TCATAACACACACCATCTTCTGGTTGAGAGGAATATTATATATGATATTAAGGG |
| | | | | AGGAGCATTTTT |
| | | | | TATAGAAGATGGTATTGAACATGGCAATATCCTCCAGTATAACTTGGCAGTATT |
| | | | | TGTACAGCAAA |
| | | | | GTACCAGTCTTCTGAATGATGATGTGACCCCGGCTGCATTTTGGGTCACCAACC |
| | | | | CGAACAATACC |
| | | | | ATACGACACAATGCTGTTGCTGGTGGCACTCACTTTGGCTTTTGGTACCGGAT |
| | | | | GAACAACCACCCT |
| | | | | GATGGGCCATCCTATGACAGAAACATTTGTCAAAAAAGAGTTCCCCTTGGCGA |
| | | | | ATTTTTTAACAAT |
| | | | | ACTGTCCATTCTCAAGGTTGGTTTGGAATGTGGATCTTTGAGGAATATTTCCCC |
| | | | | ATGCAAACGGG |
| | | | | ATCTTGTACATCTACAGTGCCTGCACCTGCAATATTTAACTCACTTACTACTTGG |
| | | | | AATTGTCAAAAA |
| | | | | GGAGCTGAATGGGTCAATGGAGGTGCCCTTCAGTTCCATAACTTTGTGATGGT |
| | | | | GAATAACTATGA |
| | | | | GGCTGGAATTGAGACTAAGAGGATCCTGGCTCCTTATGTTGGAGGGTGGGGT |
| | | | | GAAACCAATGGA |
| | | | | GCGGTGATTAAAAATGCCAAAATAGTCGGCCATCTTGATGAACTGGGAATGG |
| | | | | GGTCTGCATTTTG |
| | | | | CACAGCAAAAGGCCTGGTTCTCCCATTTAGTGAAGGCTTGACTGTCTCTTCTGT |
| | | | | GCACTTTATGAA |
| | | | | CTTTGACCGTCCCAACTGTGTAGCTTTGGGAGTGACATCCATCTCTGGAGTTTG |
| | | | | TAATGACAGATG |
| | | | | TGGGGGTTGGAGTGCAAAGTTTGTTGACGTCCAGTATTCTCACACACCGAACA |
| | | | | AGGCTGGCTTTC |
| | | | | GCTGGGAACATGAAATGGTAATGATTGATGTTGATGGCTCACTTACAG |
| 93035 | PKHD1L1 | HSX2_02152308 | 653 | GGCACAAAGGACATACCGTCATTCCACACAGCTCATTGCTAGACCCTTCTCATT |
| | | | | GTACTCAGGAA |
| | | | | GCTGAGTGGAGCATTGGGTTCCCTGGATCAGTCTGTGATGCTTCAGTCAGCTT |
| | | | | TCACCGTTTAGC |
| | | | | GTTCAACCAGCCTTCTCCAGTATCTCTGCTTGAAAAGGATGTGGTTCTTTCAGA |
| | | | | CTCTTTTG |
| 93035 | PKHD1L1 | HSX2_02152318 | 654 | GCACAAGCATTATTCCATTTCAGAAGAAACGACTGACTCATATGTCTGGATGG |
| | | | | ATGGCTCTGATTC |
| | | | | CAAATGCAAATCACATTAACTGGTATTTTAAAGGTGTGGATCACATAACCAAC |
| | | | | ATTTCATATACAT CGACATTCTATGGATTCAAG |
| 93035 | PKHD1L1 | HSX2_02152328 | 655 | GAAGAAGACTATGTAATTATATCACATAACTTCACTCAAAATCCTGACATGTTT |
| | | | | AATATTATTGATA |
| | | | | TGAGGAATGGTTCCTCAAATCCATTGAATTGGAATACTAGCAAGAATGGGGAC |
| | | | | TGGCACCTTGAA GCAAACACTAGTACTCTATATTACTTGG |
| 93035 | PKHD1L1 | HSX2_02152339 | 656 | TGTCAGGAAGAAATGACCTTCATCAGAGTCAGCTCATTTCTGGGAACCTGGAT |
| | | | | CCTGATGTGAAA |
| | | | | GACGTTGTTATTAATTTCCAAGCTTACTGTTGTATTCTCCAGGATTGCTTTCCTG |
| | | | | TACATCCGCCAT CAAGAAAACCAATTCCCAAGAAGCGACCAGCCACATATAA |
| 93035 | PKHD1L1 | HSX2_02152370 | 657 | GGAGGTAGATTAATCGGTGGCTGGGAAGATAACCCTTTTAAAGGAGACTTAA |
| | | | | AGATTGTTCTTAG |
| | | | | AGGAAATCATACTACACAAGACTGGGCTCTTCCAGAAGGACCAAATCAAGGG |
| | | | | GCAAAGGTCTTA G |
| 93035 | PKHD1L1 | HSX2_02152379 | 658 | GGGTGTTTGGTGAGCTGGATCTTCATGGAATTCCACATTCAATATATAAAACTA |
| | | | | AGCTCTCAGAAA |
| | | | | CTGCATTTGCAGGTTCCAAAGTCCTGTCTCTGATGGATGCTGTGGATTGGCAG |
| 93035 | PKHD1L1 | HSX2_02152389 | 659 | GAGGGAGAAGAGATTGTGATAACAACCACAAGCTACGATTTCCACCAGACAG |
| | | | | AAACAAGAAGTA |
| | | | | TCGTTAAAATCCTGCATGATCATAAAATTCTCATTCTTAATGATAGCCTTTCCTA |
| | | | | TACTCACTTTG |
| 93035 | PKHD1L1 | HSX2_02152399 | 660 | CTGAAAAATACCATGTCCCTGGAACTGGTGAGAGCTACACGTTAGCAGCTGAT |
| | | | | GTTGGGATACTG |
| | | | | AGTAGGAACATCAAAATAGTTGGTGAAGATTACCCCGGTTGGTCTGAGGACTC |
| | | | | TTTTGGAGCACG |
| | | | | CGTACTGGTTGGCTCATTCACTGAAAATATGATGACATTTAAAG |
| 93035 | PKHD1L1 | HSX2_02152410 | 661 | GAAATGCAAGAATAAGTAATGTGGAATTTTATCACAGTGGTCAAGAAGGCTTC |
| | | | | AGGGATAGCAC AGATCCAAGATATGCTGTAACGTTTCTTAACCTAGGACAG |
| 93035 | PKHD1L1 | HSX2_02152474 | 662 | AGCTCATTAATTGTTGGAAGTAGCCCTGGGTTTAATTGCTCTGATGTCCTAACT |
| | | | | AATGATGATCCT |
| | | | | AATATTGAACTCACTGCTGCTCATCGGAGTCCTAGATCTCCATCAG |
| 93035 | PKHD1L1 | HSX2_02152481 | 663 | GTGGGAGAAGTGGGATTTGTTGGCCTACCTTTGCTTCAGCTCATAACATGGCA |
| | | | | CCCCGAAAGCCC |
| | | | | CATGCAGGAATCATGAGTTACAATGCCATCAGTGGCCTTTTGGACATCTCAG |
| 93035 | PKHD1L1 | HSX2_02152497 | 664 | GAATTATTAGAGATTCAACCTGTAAGTACCTTCCAGAGTGGCAGAGCTATCAG |
| | | | | TGCTTTGGGATG |
| | | | | GAATATGCAATGATGGTTATTGAAAGTCTGGATCCTGACACAGAAACTCGAAG |
| | | | | ACTTTCCCCAGT GGCTATAATGGGCAACGGTTATGTTGATCTTATTAATG |
| 93035 | PKHD1L1 | HSX2_02152524 | 665 | GTGACTGCCCAGCCAGTTGAAAGGTCTGCATTTCCTGTTCATCACGTGGCCTTC |
| | | | | GTGTCCTCACTC |
| | | | | TTAGTGATCACTCAGCCGGTGGCAGCACAGCCAGGACAGCCATTTCCTCAGCA |
| | | | | GCCTTCGGTAAA GGCAACAGATTCTGAC |
| 93035 | PKHD1L1 | HSX2_02152529 | 666 | GGTAACTGTGTATCAGTTGGAATTACTGCACTAACTTTGAGGGCCATACTCAA |
| | | | | GGACTCCAATAAT |
| | | | | AACCAAGTCAATGGCCTTAGTGGAAATACAACAATTCCGTTTAGCAGCTGTTG |
| | | | | GGCCAACTACAC AGACCTTACTCCCCTTAGAACAG |
| 93035 | PKHD1L1 | HSX2_02152541 | 667 | GAAGCTACTAAAGTGCTGTTCCGAAGAATAGGCTGAAACAAAAATATAAGAAT |
| | | | | TATTAGCTACTT |
| | | | | TGTTGGGCAATAGGCAAAAGTCTATAGCATTTTCATGAAAATATACTAAAAATA |
| | | | | TTTTTATGATAT |
| | | | | ATAAAATGTACTAATTAGCTTTAAACACTAAAATCAGATTTCTTCAAAATATAA |
| | | | | ATTTGTTTTGATT |
| | | | | CTTTATATTTATATGTTTTTATTTCATTTCAATAAACTTCCAGAAATTTGTCATTT |
| | | | | GGAAGTAGATAT |
| | | | | GACACCTCTAAGTTATTGTACCAACAAATCATAGAATCCTTTAAATAATGGAAA |
| | | | | GAGCTTGTCTAG |
| | | | | TTCCGACTTAAGCTCTTTACATCAGATTGGAAGCATTTTAAGTATTCTTTTTAAT |
| | | | | ACTTGAGGGGG |
| | | | | GGGGTTCTTATTTTCTCTATCCATTTACAAGGTTTTGATTTCCAATAATCTAAGC |
| | | | | CATTCTAGTTTCT |
| | | | | AAGGATTTTGGAGAGACGAGATTGTCTTCACACAATCTGAGATGACAATCTCA |
| | | | | TCTCAGATTTTTC |
| | | | | ACACTGAGTTCCTGTCTTTCAGATTCACAGTAATCCCAGGAATTCTAGACTGTC |
| | | | | CTGTATTTCTGG |
| | | | | ATCTGCCCAACTCTGGCCAATATTGGGAAAAGTTTTCTTGTCCTTGGAATGAAC |
| | | | | TTTTGGAACAAG |
| | | | | TCCAAACTCCTTCTCTGCCTACCCCTCCTTCCCACTCAAAACTGCCACTACTAAG |
| | | | | AGCAGGACTCCC |
| | | | | CTCAACCCCGGGCATGAGTTGAGGGTTGAGGGGTTCCCTTAACTGGTGGATGC |
| | | | | TTATTCACTGTT |
| | | | | TACTGGATGAATGAAGTATAAAGTGTGGGAAGTCAACAACAGAACAGAACTT |
| | | | | ATTTTCAAGAAGA |
| | | | | TAAATTAGAGAATGCAAAAAAGCTACAGACTAAGGTAGCTAAGTTAACCGAAC |
| | | | | TCTCTAACAGTA |
| | | | | TTGAAAATAGCAATTCTTTACTCAGAAAATTCTAAAGGGAATACTTAATTTGAC |
| | | | | AGAACTCCTAAT |
| | | | | AAAGACATTGTAGCCAGATCCAGAGCCTTCCAGCAAGTGACACTCAGCAAACA |
| | | | | TTTGGGACAGCT |
| | | | | GAGAGAATTCAACAAAGCCCATACCGTTGTCTCTTGTATAACATCTCCTAGTAC |
| | | | | ATTCACCTCAGA |
| | | | | TCATTGTTAGTCTTACTTAGCAGAAAATGTATAAACACTTACCAGGTAGTAAA |
| | | | | ATCAAATATCTTG GTA |
| 5315 | PKM | HSX2_01127816 | 668 | CGGCGGCCCGCAGCGGGGCTCCTTGCACAGCAGC |
| 22925 | PLA2R1 | HSX2_01578093 | 669 | AGGTAAATTTTGAGCCATTTGTTGAAGATCACTGTGGAACATTTAGTTCATTTA |
| | | | | TGCCAAGTGCCT |
| | | | | GGAGGAGTCGGGATTGTGAGTCCACCTTGCCATATATATGTAAAAAATATCTA |
| | | | | AACCACATTGAT CATGAAATAGTTG |
| 22925 | PLA2R1 | HSX2_01578104 | 670 | AAAAAGATGCGTGGAAATATTATGCTACCCACTGTGAGCCTGGCTGGAATCCC |
| | | | | TACAATCGTAAT |
| | | | | TGCTACAAACTTCAGAAAGAAGAAAAGACCTGGCATGAGGCTCTGCGTTCTTG |
| | | | | TCAGGCTGATAA |
| | | | | CAGTGCATTAATAGACATAACCTCATTAGCAGAGGTGGAGTTTCTTGTAACCCT |
| | | | | CCTTGGAGATG |
| 22925 | PLA2R1 | HSX2_01578263 | 671 | ATATTCACACTGCAGAGGCGCTGCCAGAAAAAG |
| 22925 | PLA2R1 | HSX2_01578389 | 672 | AAAACTCTGAAATCAATGCGAATAATAGTTTTTGCATTAATGTATCTCTACTAA |
| | | | | AATTTGGGGGAA |
| | | | | TTTTAAAACTAATCTGGTATCTATTCAGACATTTACCTGCACTCGTACCATTAAG |
| | | | | AAAGACAGAAA |
| | | | | GAAGCAAAAAAAAATTAATCTTGTATATGAGAGGAAAAGGAAAGGGCTTCTG |
| | | | | AGAGGATTCTTA |
| | | | | GTTGTTTCTTTTGAATTCACTTTTAATAGCAGGATTTGGAAAATACTAATTTCTG |
| | | | | TGCTTAAGGGTC |
| | | | | ACAGGTTCTGGGCTCTCAACTGATATTTAAGGTGACATTCATTTTTATTAGGTCT |
| | | | | AACATCTCAAG |
| | | | | CTAAAGGAGAAAGAAAAATACCTTCTTTTAAATGGCAAAGACCTTCATTAGCA |
| | | | | GCACACTTTTATA |
| | | | | AACAACCATATAGTTAAAATGTGGCCTTAAACTTTCAATTACTAAATGATGATT |
| | | | | AAGTTGGATATTT |
| | | | | TAAAATGTCTTATACATAAGCTTAAAGTAATATATTGAAACTTTAACAGTTGTA |
| | | | | CTGTTAAAACTCA |
| | | | | TGGACTTTCTGAAAAACTAAATATATAAAATAATATGTAACACTCTTAATAACT |
| | | | | GGAAGATATAAA |
| | | | | ATGTAACTTGGATTTAAAGGAGAAGAGCTAAAGATCTGAATTATAGTCTCATT |
| | | | | AGAACCAGACAG |
| | | | | ATGTTGTTGAGAAGTACTAAATAAATTAGTAATCTAGATATCTTTATTATGTAA |
| | | | | ATGAGTTTAGGT GTTCTATTTAATAAACTATTTTCTGG |
| 57664 | PLEKHA4 | HSX2_01939952 | 673 | AATCCGCCCAAAAGCGGCAGCCAATCGGAGCGCGAGGACGTGGTCTGGAGGT |
| | | | | ACCGCCGGAGA |
| | | | | TCTGGGACCACTCAGGGCATCAGGGGGCGTGGTCTGGTCCCCATTGCGGGCC |
| | | | | CGGGAGGGGAA |
| | | | | TGGTTTCTATGGCCAAAGTTTGGTTTTCTCAACACTGTCTAAATTTGGATTAAAA |
| | | | | CTTTGAACTTTT A |
| 57664 | PLEKHA4 | HSX2_02562176 | 674 | GTGGAAATTTGGACTCCCAGGGAGACCCTCTTCCCGGTGTGCCGCTGCCTCCTT |
| | | | | CGGACCCCACG |
| | | | | CGCCAGGAGACCCCTCCCCCCAGATCTCCCCCGGTGGCTAATTCGGGTTCCAC |
| | | | | GGGGTTCTCTCG |
| | | | | CCGAGGGAGTGGGCGTGGAGGAGGTCCCACCCCCTGGGGGCCCGCGTGGGA |
| | | | | TGCCGGGATCG |
| | | | | CCCCTCCGGTCCTGCCACAAGACGAGGGGGCATGGCCTCTGCGAGTCACTCTG |
| | | | | CTACAATCCAGC |
| | | | | TTCTAATCCGCCCAAAAGCGGCAGCCAATCGGAGCGCGAGGACGTGGTCTGG |
| | | | | AGGTACCGCCGG |
| | | | | AGATCTGGGACCACTCAGGGCATCAGGGGGCGTGGTCTGGTCCCCATTGCGG |
| | | | | GCCCGGGAGGG |
| | | | | GAATGGTTTCTATGGCCAAAGTTTGGTTTTCTCAACACTGTCTAAATTTGGATT |
| | | | | AAAACTTTGAACT TTTA |
| 5362 | PLXNA2 | HSX2_01133693 | 675 | TGGCCACACACCCCTGACCATCACAGGCTTCAACCTGGATGTCATTCAGGAGCC |
| | | | | AAGGATCCGAG TCAAATTCAATGGCAAAGAATCTGTCAAT |
| 55558 | PLXNA3 | HSX2_01855156 | 676 | TGGAAGCACTGCCATCACTGTGAGTGGGACCCACCTGCTGACGGTCCAGGAG |
| | | | | CCCCGGGTCCGT GCCAAGTACCGCGGCATTGAGACCACCAAT |
| 5365 | PLXNB3 | HSX2_02513200 | 677 | TGATCCTGTACGACTGCGCCATGGGCCACCCGGACTGCAGCCACTGCCAAGCG |
| | | | | GCCAACAGGAG |
| | | | | CCTGGGCTGCCTGTGGTGTGCTGACGGCCAGCCTGCCTGTCGCTATGGGCCCT |
| | | | | TGTGCCCGCCG |
| | | | | GGGGCTGTGGAGCTGCTGTGTCCTGCGCCCAGCATTGATGCA |
| 5371 | PML | HSX2_01134444 | 678 | CATCCATCCCACCTGCCACTACCTTGGGTGTCCTTACAGCTCTGCCCTGACCCCA |
| | | | | GCCCCT |
| 10585 | POMTT | HSX2_01513339 | 679 | AAGTACATGGGTGTGTTCACGTACGTGCTCGTGCTGGGTGTTGCAGCTGTCCA |
| | | | | TGCCTGGCACCT GCTTGGAGACCAGACTTTGTCCAAT |
| 10585 | POMTT | HSX2_02537077 | 680 | CATCAAGTACATGGGTGTGTTCACGTACGTGCTCGTGCTGGGTGTTGCAGCTG |
| | | | | TCCATGCCTGGC ACCTGCTTGGAGACCAGACTTTGTCCAAT |
| 64091 | POPDC2 | HSX2_02563563 | 681 | CGGGCCAGGTTGTCCAGGTCACTCTGACTGCTGA |
| 25833 | POU2F3 | HSX2_01660779 | 682 | TAACGTCATCCTGTTCCCCTGGGAACAACAGCAGGCCTTCATCTCCTGGCTCAG |
| | | | | GACTCCACGCC |
| | | | | AGCAGCCCCACTGCATCTCAAAATAACTCCAAAGCAGCAGTGAACTCCGCCTC |
| | | | | CAGTTTTAACTCT TCAGG |
| 25833 | POU2F3 | HSX2_01660796 | 683 | ATCTTGGTACCGATGGAATCATTCCACCTACCTCCACTGAGACCAAAAAGTTTC |
| | | | | TCCTACTCCAGC TGGCCCTGTATTCCCCTGG |
| 5504 | PPP1R2 | HSX2_01142158 | 684 | GGAAGAATCAAATCAAGGATCTACTCCAAGTGAC |
| 129285 | PPP1R21 | HSX2_02204481 | 685 | TGCACAGAGCCGCAGGGCTGAGACCACGTCCATG |
| 5567 | PRKACB | HSX2_01146811 | 686 | GGCTACAATAAGGCAGTGGATTGGTGGGCATTAGGAGTGCTAATCTATGAAA |
| | | | | TGGCAGCTGGCT |
| | | | | ATCCCCCATTCTTTGCAGACCAACCAATTCAGATTTATGAAAAGATTGTTTCTG |
| | | | | GAAAG |
| 222171 | PRR15 | HSX2_02304267 | 687 | CACAGGCCGCTGCCTCAGCATCCACCCCGCAGCCCACGTGTGGCAAGCCGGG |
| | | | | GAAGGGGTGGA |
| | | | | GTGAACGGCCGGAGACCACGTGGAGAAAGGGGCCGCTTTGGCCCTTCCATCT |
| | | | | GGGTGCCGGGA |
| | | | | GCCCCTAGGCCCTCCGGCCATGGCCGACAGCGGCGATGCTGGCAGCTCCGGC |
| | | | | CCCTGGTGGAAA |
| | | | | TCGCTCACCAACAGCAGAAAGAAAAGCAAGGAAGCCGCAGTGGGGGTGCCG |
| | | | | CCTCCCGCCCAG |
| | | | | CCCGCTCCCGGGGAGCCCACGCCACCTGCGCCGCCCAGCCCGGACTGGACCA |
| | | | | GCAGCTCCCGGG |
| | | | | AGAACCAGCACCCCAATCTCCTCGGGGGCGCCGGCGAGCCCCCCAAACCAGA |
| | | | | CAAGTTATACGG |
| | | | | GGACAAATCCGGCAGCAGCCGCCGCAATTTGAAGATCTCGCGCTCCGGCCGCT |
| | | | | TTAAGGAGAAG |
| | | | | AGGAAAGTGCGCGCCACGCTGCTCCCGGAGGCGGGCAGGTCCCCGGAGGAG |
| | | | | GCAGGCTTTCCT |
| | | | | GGTGACCCCCACGAGGACAAGCAGTAGCCCCAATAGCCTGCGCGCTCCAGGA |
| | | | | CTGCCTACCCAG |
| | | | | CACTACCCCAAACCCCCAGTTCCAAACCCGAGACTTCAGGCCCGCCCCCTTACG |
| | | | | CGTTGTCTCATT |
| | | | | CCACCAAATTCAGAATATTTACACAATGCCTTCATGATTTTATTTTTCTGGAAAT |
| | | | | TGAAGTGTCAAT |
| | | | | TGGGTTCTCAATATTTCATGACTCCAAGGATGCATTAAATATTTATTTGTGGTA |
| | | | | AGAGAAGATACC |
| | | | | TGCCGCGGAGGAGGGTGGCATAATTATTTTTTTTTCAAAAAGCTATTCTGGGG |
| | | | | CCCTGGCCCCCT |
| | | | | GGCTCCTGTCACAGCTCAGCTTGAACTCTGTAGCCTCTCAAATGAAGAAGGTG |
| | | | | GCTGTTATTTAG |
| | | | | GACTCTGTGGAAAGCAAATCACAGTGCTGTTTTTAATGCCTGAGAAATGCACT |
| | | | | TTAGGGGTAGAC |
| | | | | TTATACCTTAAGTGAAGGAGTGGGGGAGGAAGAAGCATTCTTAAGTAGAAGC |
| | | | | ATTTTAAAATAA |
| | | | | GGCAGGATTGCTTTTAATGTTAAACTACAAAACTGTACAGCCTATTTTAGTGTG |
| | | | | GACTATTAAAAC CCTTGCACTGTAAGG |
| 11098 | PRSS23 | HSX2_01548939 | 688 | CTTCATGTGGACCCCAGTGTCATAAGGGAACTCCA |
| 23362 | PSD3 | HSX2_01629459 | 689 | GATCAGCGAACAAGAGGAGCACGTTAAGGGGGAAGATGAAGACATCCTTGG |
| | | | | GCCTGGATATAC |
| | | | | GGAGGACTCCACCGACGTGTACAGCTCCCAGTTTGAAACCATTTTGGACAACA |
| | | | | CTTCTTTATACTA |
| | | | | CAGTGCAGAGTCCCTGGAGACATTATACTCAGAGCCTGATAGCTATTTTAGCTT |
| | | | | TGAAATGCCCCT |
| | | | | CACTCCAATGATACAACAGCGCATTAAAGAAGGTGGTCAGTTCTTGGAGAGGA |
| | | | | CATCAGGGGGA GGACATCAGGATATCCTG |
| 23362 | PSD3 | HSX2_01629461 | 690 | AGTGTGTCTGCAGATGGTGGCATCGTGATGGGCTATTCTAGTGGCGTCACCAA |
| | | | | TGGGCTGAATG |
| | | | | ATGCCAGCGACTCCATCTACACGAAAGGCACCCCGGAGATTGCTTTCTGGGGA |
| | | | | AG |
| 23362 | PSD3 | HSX2_01629472 | 691 | GAGAGAGACAGGGAAAGGATCAGCGAACAAGAGG |
| 23362 | PSD3 | HSX2_01629478 | 692 | CAATGCTGGGGTGAAAACAACACGGCTAGAAGCTCATTCTGAAATGGGGAGC |
| | | | | ACTGAAATTTTG |
| | | | | GAAAAGGAGACCCCAGAAAATCTCAGTAATGGTACCAGCAGCAATGTGGAAG |
| | | | | CAGCCAAAAGGT |
| | | | | TGGCCAAACGCCTTTATCAGCTGGACAGATTCAAAAGATCAGATGTTGCAAAA |
| | | | | CACCTTGGCAAGAA |
| 23362 | PSD3 | HSX2_01629528 | 693 | ATGGAGTCCATTGCCTTACCTGTGCAATAATGCTTCTTAATACCGATCTACATG |
| | | | | GCCAC |
| 23362 | PSD3 | HSX2_01629587 | 694 | AGATGATGAAGAGAAAAAAAAGTCTCCCTCAGAAAGTACTGAGGAGAAAGCT |
| | | | | AACGGAACACAT |
| | | | | CCAAAGACCATCAGTCGTATTGGAAGTACTACTAACCCATTTTTGGACATTCCT |
| | | | | CATGATCCAAAT |
| | | | | GCTGCTGTGTACAAAAGTGGATTCTTGGCTCGGAAAATTCATGCAGATATGGA |
| | | | | TGGAAAGAAGA |
| 23362 | PSD3 | HSX2_01629635 | 695 | GAGGAGCAACTGAAGTCACATGAAAGTAAGCTGAAGCAGATCACCACCGAGC |
| | | | | TGGCCGAGCACC |
| | | | | GCTCATATCCCCCCGACAAGAAGGTCAAAGCCAAGGACGTCGATGAGTACAA |
| | | | | ACTGAAAGACCA CTATCTGGAGTTTGAG |
| 23362 | PSD3 | HSX2_01629644 | 696 | AAAACCCGCTATGAAATGTATGTCAGCATTCTCAAGGAAGGAGGCAAAGAGC |
| | | | | TACTGAGTAACG |
| | | | | ATGAAAGCGAGGCTGCAGGACTGAAGAAGTCGCACTCGAGTCCTTCGCTGAA |
| | | | | CCCGGATACTTC |
| | | | | TCCAATCACTGCCAAAGTCAAGCGTAACGTGTCAGAGAGGAAGGATCACCGA |
| | | | | CCTGAAACACCA |
| | | | | AGCATTAAGCAAAAAGTTACTTAGAGTCCATCTGCGGCCAGGAAGTGCTGGTC |
| | | | | ATGGAG |
| 23362 | PSD3 | HSX2_01629645 | 697 | CAAAACGGTGCATTAGTAATTTTTTCTATTGTATATTTTTGTTAGTTTCTGTACA |
| | | | | GATTGTCTTTGCT |
| | | | | CTTGATTTCTTTTGCTTTGATGATTTTTGCAACTTGATAGCTAATGCACCTTTTCT |
| | | | | GTGAGGAGGAG |
| | | | | GGGATCGTGATTTCAGAATGAATTATGTATCCCTTCTCTTTTGGTTTTCTCTTGT |
| | | | | TTGCAGTCTGCT |
| | | | | CAGTTGTTTTATGTATTCTCATATCAACTGTTAAACTTTTTTTTAAGGTTAAAGA |
| | | | | ATTTAATCCATTG |
| | | | | TGAAACACTTAACTGGACAAACTGTAGTTTTAGTAAATTCTAGCTGGAGTTAAT |
| | | | | ATACGCCTTTAT |
| | | | | ATGTGAAATCTTGCCCAGTCACAGAGGTAGAATTGAGCACTCACAGATGCTCC |
| | | | | AGTAAGAATCAC |
| | | | | AGTGCTGGGAATCTAGTTGCTCCAATATGAGGCAGCTTCATGTGCAGCTTAGC |
| | | | | ACTTGTTGTTGA |
| | | | | GATCGGACCCTGCTGGAAGCAGGGAAAAGAAGCGTGAAGATCGTAGGATTG |
| | | | | AGAACTTAGGGA |
| | | | | AGCACATTAGCTTGCTTGAAGTGCTGATTCCATTTCAGCCAAGCAAGGGAAAG |
| | | | | AGGAAGTGGAG |
| | | | | TCATTTTGCCTTTGAAGGCTGAGGAAAGATTGATACCCAGTTAATTTTGTTTGC |
| | | | | TAAAGGATGGG |
| | | | | GGCAATAATCGGCCCTTGAGGAGCTGCAGCAGTAGGCATGTGCTCAGTCTGC |
| | | | | AGGAATTGTTAC |
| | | | | CTCACTCCCACAGGGTCTAGACTAGAAATCCATCATCTCTATCGTTGATATCCTT |
| | | | | CCATCCAGGAA |
| | | | | TAGATTTTTCTTACTCTACATATGTGTGTGTGCGTGCGTGTGTGTGCGTGTGTG |
| | | | | GGCATGGGGTT |
| | | | | GTGTCCTGGTTGTGATATTGAGGTCTTCCTTCCTAACAAATTAATACTAAAATG |
| | | | | AAACAGCTTTTCT |
| | | | | TGTGTCCTTAAGACAAAATAAGGAAGGAAAACGTAGCTGCAGTTGTCCACGAT |
| | | | | GGATATTGGTTC |
| | | | | TTTAAAATATATCTGAAAGTAGTAGTCAGAATGAATTATGGTTGGAAAACTGA |
| | | | | GGAATCTTCTGG |
| | | | | TTGCAGGTGCAAAGTGACTTTGTTTATTCTTGTCTCAGTCTCCTTGATAGCCACT |
| | | | | TCACTCTGCTAC |
| | | | | TACTCAACTTTCTCCTAAAAATACTTCATCTATTTTCAGTCCTTTCTTTCTGTCTA |
| | | | | CTCAAAATGGTT |
| | | | | CTATTAACTTTGCAGTCATGAGCTTGTTCCAGTTACAGTCCCTTTGAAGTTCAG |
| | | | | GGTGATAAACAG |
| | | | | AATATTCTTCTGTAGAGGAAGAGAAAGGAGTGAAAGTTTAGCCCACTGAGAC |
| | | | | CTAGAGCTTTGTG |
| | | | | ATTTCCTAACCTTGAAACTCTGTAATCCCTAAAGTTAAAATCTCCGCAAGTGGC |
| | | | | ACAACTTCAGAA |
| | | | | CTAATAGTATCACTTTGATTTTTCTTTTTCCTCCCTTAGAAAGTTTCTCTAGTTCT |
| | | | | ATAGTTTATTTGT |
| | | | | TGAAGGTACTATGACCAAAGAATCAGCTGCTCTACAGGAATAGCATGGTTCCA |
| | | | | GTGAATTAGAGA |
| | | | | AAACCTGCTGTAAAGCCATGGTAGTGTCTAAGTGGTATGTTATTATGATGTACT |
| | | | | AGCATTTATTTA |
| | | | | CAGAATTATTTATTAACGTTTACTTCCTTCCCCTCTGTAAATGTCCATGACTATT |
| | | | | GCCCAGAGAAGG |
| | | | | CTTACCCCTCTCTAGGGTTGCAGTTGCTTTCTTTGTAATAAGTATTTTGCCACAC |
| | | | | CTGTAAAAAAAA |
| | | | | AAACCTCACTTTTAACTCTCTGCCTTGTTTGGGTAAAGGCAGTAACTAAGTTTA |
| | | | | TGTTTCAGAACT |
| | | | | GCAAAACAAACAGGATAGTTACCAATATGGCCCATGTATCAGATTGATTTTTGT |
| | | | | AGCCTCTCACTG |
| | | | | AATCCAACATATCCACAAGCAAGTTATCTGTCTTTCTACCTGATAATCTAAATTA |
| | | | | TCAGGATATTTG |
| | | | | TTTTCTGCCTAAATGTTTATACTAAGCCGAGGGGAGAGAGGTACCTAGACCAT |
| | | | | GTCATCTACAAG |
| | | | | CTTCAGTAACTAAAGAAAAAGGAACTTCCCTGAGTGGCTTGAATGTGTTTGCC |
| | | | | CACAGTCTATATC |
| | | | | TATGTATATAGAATGTCTGTATGTATTTTACTTATTTAATATACATTGAATGGTA |
| | | | | CCTTGCTACAGT |
| | | | | ATTTCTGACATTTAGAGTAGTGTTGAAATACTCGGCTAGCATCAGCACCACTAT |
| | | | | AGCACTGTCCGT |
| | | | | GTCATATGAGTCACTAATATTAACTCCAGGGACTTCTGGATAGGCTAATAGATC |
| | | | | ATTGGATACGA |
| 5725 | PTBP1 | HSX2_02515245 | 698 | GCCTTTCCGTTCCGAACGTCCACGGCGCCCTGGCCCCCCTGGCCATCCCCTCGG |
| | | | | CGGCGGCGGCA |
| | | | | GCTGCGGCGGCAGGTCGGATCGCCATCCCGGGCCTGGCGGGGGCAGGAAAT |
| | | | | TCTGTATTGCTGGTCAGCAACCTCAACCCAGAG |
| 5747 | PTK2 | HSX2_01163391 | 699 | TTGCCATCAATACCAAAGTTGGCCAACAGCGAAA |
| 5786 | PTPRA | HSX2_01166131 | 700 | ATGACCACTCTAGAGTCCACCTGACACCGGTTGAAGGGGTTCCAGATTCTGAT |
| | | | | TACATCAATGCTT CATTCATCAAC |
| 5793 | PTPRG | HSX2_01167541 | 701 | ACAGCGACAAAGACTTGAAAGCCACCATTAGCCA |
| 25797 | QPCT | HSX2_01658589 | 702 | ACTGTTTCAGACTCCAAGCCAGATTTGTCACTCCAGCTGATCTTCTTTGATGGT |
| | | | | GAAGAGGCTTTT |
| | | | | CTTCACTGGTCTCCTCAAGATTCTCTCTATGGGTCTCGACACTTAGCTGCAAAG |
| | | | | ATGGCATCGACC |
| | | | | CCGCACCCACCTGGAGCGAGAGGCACCAGCCAACTGCATGGCATG |
| 5873 | RAB27A | HSX2_01173704 | 703 | GAGGAAGCCATAGCACTCGCAGAGAAATATGG |
| 5873 | RAB27A | HSX2_01173714 | 704 | AATTAATTTGTTGCAGCTTTGTAAATATTTCTTTAAGATTCAGCCTGAGAGTTA |
| | | | | GGAGAAATATTTC |
| | | | | AGAGCCAAAAGTGCCTTATACAACCTTAGCCTATTATAGTAAATCATTCAAGGA |
| | | | | TTCAGAATTTTG |
| | | | | CAGTCACAGAAGAGTGTATTTATTATGTAGAATGAATGAGGGTACTGTCACCT |
| | | | | GCCTTAATGTAG |
| | | | | GTAGGCCCAGAGTCTTACATTTAAGATCTTACATGCAGTTATAAAACCGCCACA |
| | | | | GTCTTCAATCCA |
| | | | | GATTTGAAGACTCATGCCATAGGTGACATTCTAAAATACCATTAAAGCCACTT |
| | | | | AAATGTTAAATAA |
| | | | | GAATATACATGCACATCAGCTCAATGTCTTTGAGTATTAATTTTATGTAAGCAT |
| | | | | TCTATTTAACATG |
| | | | | AATATAGGACAAATCATGGCTATATCTATAGACCTTGGATAAACTGGATTGAC |
| | | | | CAATTATACACTC |
| | | | | ACGGTGACTTTTTTATTGGTGGGAAGGGGATTGGGGTGGGGCAGGCTGGCTT |
| | | | | AATGTAATATGA |
| | | | | GCAACCAAAGTGGGACTTCTGTCTCCCCGCTATATTCCCATTGCTCTGAATGGT |
| | | | | TGATTGAAGGGT |
| | | | | CAGGGAACTAGATTTTATGGCTTTAGTTCACTGTGATTGTACATTTATACTTGG |
| | | | | CCTATGTGCTGG CCGCACCTGAACATAGCTGG |
| 7033 | TFF3 | HSX2_01262852 | 705 | CTGCAAACCAGTGTGCCGTGCCAGCCAAGGACAGGGTGGACTGCGGCTACCC |
| | | | | CCATGTCACCCC |
| | | | | CAAGGAGTGCAACAACCGGGGCTGCTGCTTTGACTCCAGGATCCCTGGAGTG |
| | | | | CCTTGGTGTTTCA AGCCCCTGCAGGAAGCAG |
| 5887 | RAD23B | HSX2_01174939 | 706 | GGAATCCCTGGAGATAGAGAAAGTCAGGCTGTGGTTGACCCCCCTCAAGCAG |
| | | | | CTAGTACTGGGG |
| | | | | CTCCTCAGTCTTCAGCAGTGGCTGCAGCTGCAGCAACTACGACAGCAACAACT |
| | | | | ACAACAACAAGT TCTGGAG |
| 5887 | RAD23B | HSX2_02516158 | 707 | TGACGGGTCAGTCTTACGAGAATATGGTAACTGAGATCATGTCAATGGGCTAT |
| | | | | GAACGAGAGCA |
| | | | | AGTAATTGCAGCCCTGAGAGCCAGTTTCAACAACCCTGACAGAGCAGTGGAGT |
| | | | | ATCTTTTAATG |
| 26953 | RANBP6 | HSX2_01694312 | 708 | GTGAGACAAATGGCTGCCGCACTGCTACGACGGCTTTTGTCCTCTGGGTTTGA |
| | | | | GGAGGTTTATCC |
| | | | | AAATCTGCCTGCTGATGTTCAGAGAGATGTCAAGATTGAACTGATTCTGGCTG |
| | | | | TTAAGTTAGAAA |
| | | | | CACATGCTAGCATGAGGAAAAAACTTTGTGATATTTTTGCAGTGCTGGCCAGG |
| | | | | AATTTGATAGAT |
| | | | | GAGGATGGCACTAACCACTGGCCGGAAGGTCTGAAGTTTCTTATTGATTCAAT |
| | | | | CTACTCCAAAAA |
| | | | | TGTGGTTCTATGGGAAGTTGCACTTCACGTTTTCTGGCACTTTCCTGGGATTTTT |
| | | | | GGGACCCAAGA |
| | | | | GCGGCATGATTTGGATATCATCAAACGGTTGTTGGACCAGTGTATTCAAGATC |
| | | | | AAGAACATCCAG |
| | | | | CAATCAGGACATTATCCGCTAGAGCTGCAGCTGCATTTGTACTTGCTAATGAGA |
| | | | | ATAATATTGCTC |
| | | | | TTTTCAAAGACTTTGCAGACTTGCTTCCTGGAATCTTACAGGCTGTGAATGACT |
| | | | | CATGCTACCAGG |
| | | | | ATGATGATTCAGTGCTAGAATCCCTTGTTGAGATTGCAGATACCGTACCTAAGT |
| | | | | ACTTGGGTCCTT |
| | | | | ATTTAGAAGATACTCTACAGTTGAGTTTGAAGTTATGTGGAGACTCTAGGCTTA |
| | | | | GTAATCTGCAGC |
| | | | | GCCAGCTGGCCCTCGAAGTTATAGTGACCTTGTCTGAAACTGCCACTCCGATGT |
| | | | | TGAAAAAACAT |
| | | | | ACAAATATTATTGCACAGGCAGTTCCTCATATATTAGCAATGATGGTTGATCTA |
| | | | | CAAGATGATGAG |
| | | | | GACTGGGTAAATGCTGATGAAATGGAAGAAGATGATTTTGACAGCAATGCAG |
| | | | | TTGCTGCGGAGA |
| | | | | GTGCACTAGACAGACTGGCTTGTGGGCTTGGTGGAAAAGTTGTTTTACCAATG |
| | | | | ACCAAGGAGCAT |
| | | | | ATCATGCAGATGCTTCAGAGCCCTGACTGGAAGTATCGACATGCTGGATTAAT |
| | | | | GGCCTTATCTGC |
| | | | | CATTGGAGAAGGATGCCATCAACAAATGGAATCAATTCTAGATGAAACAGTTA |
| | | | | ACTCCGTTTTGC TTTTTCTTCAG |
| 51195 | RAPGEFL1 | HSX2_01752559 | 709 | GATTCCAGAGGAGAACCAGCCACCCAGCAAGCAGGTGAAGCCACTCTTCCGCC |
| | | | | ACTTCCGCCGG |
| | | | | ATAGACTCCTGTCTGCAGACCCGGGTGGCCTTCCGGGGCTCTGATGAGA |
| 5921 | RASA1 | HSX2_01177981 | 710 | CCAGTAGAAGATAGAAGGCGTGTACGAGCTATTCTACCTTACACAAAAGTACC |
| | | | | AGACACTGATGA AATAAG |
| 158158 | RASEF | HSX2_02259119 | 711 | AGAAGAGCAAGTTAGTACCTTGTACCAAAACATCAACCTTGTGGAGCCAAGAT |
| | | | | TAATTCAGCCAT |
| | | | | ATGAACATGTTATAAAGAACTTCATCCGTGAGATCAGACTTCAAAGCACAGAA |
| | | | | ATGGAAAATTTGGCCATTGCGGTGAAGAG |
| 158158 | RASEF | HSX2_02259164 | 712 | GAAAACCAGAAAGTTAAGAAAGACCTTTTAGAAGCACAGACAAACATAGCCTT |
| | | | | TCTTCAGAGTGA |
| | | | | GTTAGATGCTTTGAAAAGTGATTATGCTGATCAGAGTCTGAATACTGAAAG |
| 158158 | RASEF | HSX2_02259175 | 713 | GGATCTGGAAATAATCCGAGCATACACAGAAGATCGAAATAGTCTTGAGAGG |
| | | | | CAAATTGAAATA CTCCA |
| 158158 | RASEF | HSX2_02259183 | 714 | AACAGCTAACCGGAAGCTACATGACAGTAATGATGGCCTTAGAAGTGCCCTTG |
| | | | | AAAACAGTTATA GCAAGTTCAACAGATCTTTG |
| 1000 | CDH2 | HSX2_00831170 | 715 | GAACATATGTGATGACCGTAACAGCAATTGATGCTGACGATCCCAATGCCCTC |
| | | | | AATGGGATGTTG |
| | | | | AGGTACAGAATCGTGTCTCAGGCTCCAAGCACCCCTTCACCCAACATGTTTACA |
| | | | | ATCAACAATGA |
| | | | | GACTGGTGACATCATCACAGTGGCAGCTGGACTTGATCGAGAA |
| 1000 | CDH2 | HSX2_00831175 | 716 | AAAGTGCAACAGTATACGTTAATAATTCAAGCTACAGACATGGAAGGCAATCC |
| | | | | CACATATGGCCT |
| | | | | TTCAAACACAGCCACGGCCGTCATCACAGTGACAGATGTCAATGACAATCCTC |
| | | | | CAGAGTTTACTG CCATGACG |
| 1000 | CDH2 | HSX2_00831180 | 717 | TTTTATGGTGAAGTTCCTGAGAACAGGGTAGACATCATAGTAGCTAATCTAACT |
| | | | | GTGACCGATAA |
| | | | | GGATCAACCCCATACACCAGCCTGGAACGCAGTGTACAGAATCAGTGGCGGA |
| | | | | GATCCTACTGGA |
| | | | | CGGTTCGCCATCCAGACCGACCCAAACAGCAACGACGGGTTAGTCACCGTGGT |
| | | | | CAAA |
| 1000 | CDH2 | HSX2_00831185 | 718 | CCAATCGACTTTGAAACAAATAGGATGTTTGTCCTTACTGTTGCTGCAGAAAAT |
| | | | | CAAGTGCCATTA |
| | | | | GCCAAGGGAATTCAGCACCCCCCTCAGTCAACTGCAACCGTGTCTGTTACAGTT |
| | | | | ATTGACGTAAA |
| | | | | TGAAAACCCTTATTTTGCCCCCAATCCTAAGATCATTCGCCAAGAAGAAGGGCT |
| | | | | TCATGCCGGTAC |
| | | | | CATGTTGACAACATTCACTGCTCAGGACCCAGATCGATATATGCAGCAAAATAT |
| | | | | TAG |
| 1000 | CDH2 | HSX2_00831190 | 719 | ATACACTAAATTATCTGATCCTGCCAATTGGCTAAAAATAGATCCTGTGAATGG |
| | | | | ACAAATAACTAC |
| | | | | AATTGCTGTTTTGGACCGAGAATCACCAAATGTGAAAAACAATATATATAATG |
| | | | | CTACTTTCCTTGC TTCTGACAATG |
| 1000 | CDH2 | HSX2_00831195 | 720 | GAATTCCTCCTATGAGTGGAACAGGAACGCTGCAGATCTATTTACTTGATATTA |
| | | | | ATGACAATGCCC |
| | | | | CTCAAGTGTTACCTCAAGAGGCAGAGACTTGCGAAACTCCAGACCCCAATTCA |
| | | | | ATTAATATTACA |
| | | | | GCACTTGATTATGACATTGATCCAAATGCTGGACCATTTGCTTTTGATCTTCCTT |
| | | | | TATCTCCAGTGA CTATTAAGAGAAATTGGACCATCACTCGGCTTAATG |
| 1000 | CDH2 | HSX2_00831200 | 721 | GTGATTTTGCTCAGCTTAATTTAAAGATAAAATTTCTTGAAGCTGGTATCTATG |
| | | | | AAGTTCCCATCAT |
| | | | | AATCACAGATTCGGGTAATCCTCCCAAATCAAATATTTCCATCCTGCGTGTGAA |
| | | | | GGTTTGCCAGTG |
| | | | | TGACTCCAACGGGGACTGCACAGATGTGGACAGGATTGTGGGTGCGGGGCTT |
| | | | | GGCACCGGTGC CATCATTGCCATCCTGCTCTGCATCATCATCCTGCTTA |
| 1000 | CDH2 | HSX2_00831205 | 722 | TCCTTGTGCTGATGTTTGTGGTATGGATGAAACGCCGGGATAAAGAACGCCAG |
| | | | | GCCAAACAACTT TTAATTGATCCAGAAGATGATGTAAGAGATAATATTTT |
| 1000 | CDH2 | HSX2_00831219 | 723 | GGCCTTAAAGCGGCTGACAATGACCCCACAGCTCCACCATATGACTCCCTGTTA |
| | | | | GTGTTTGACTAT |
| | | | | GAAGGCAGTGGCTCCACTGCTGGGTCCTTGAGCTCCCTTAATTCCTCAAGTAGT |
| | | | | GGTGGTGAGCA |
| | | | | GGACTATGATTACCTGAACGACTGGGGGCCACGGTTCAAGAAACTTGCTGACA |
| | | | | TGTATGGTGGA |
| | | | | GGTGATGACTGAACTTCAGGGTGAACTTGGTTTTTGGACAAGTACAAACAATTT |
| | | | | CAACTGATATTC |
| | | | | CCAAAAAGCATTCAGAAGCTAGGCTTTAACTTTGTAGTCTACTAGCACAGTGCT |
| | | | | TGCTGGAGGCT |
| | | | | TTGGCATAGGCTGCAAACCAATTTGGGCTCAGAGGGAATATCAGTGATCCATA |
| | | | | CTGTTTGGAAAA |
| | | | | ACACTGAGCTCAGTTACACTTGAATTTTACAGTACAGAAGCACTGGGATTTTAT |
| | | | | GTGCCTTTTTGT |
| | | | | ACCTTTTTCAGATTGGAATTAGTTTTCTGTTTAAGGCTTTAATGGTACTGATTTCT |
| | | | | GAAACGATAAG |
| | | | | TAAAAGACAAAATATTTTGTGGTGGGAGCAGTAAGTTAAACCATGATATGCTTC |
| | | | | AACACGCTTTT |
| | | | | GTTACATTGCATTTGCTTTTATTAAAATACAAAATTAAACAAACAAAAAAACTCA |
| | | | | TGGAGCGATTT |
| | | | | TATTATCTTGGGGGATGAGACCATGAGATTGGAAAATGTACATTACTTCTAGTT |
| | | | | TTAGACTTTAGT |
| | | | | TTGTTTTTTTTTTTTTCACTAAAATCTTAAAACTTACTCAGCTGGTTGCAAATAAA |
| | | | | GGGAGTTTTCAT |
| | | | | ATCACCAATTTGTAGCAAAATTGAATTTTTTCATAAACTAGAATGTTAGACACAT |
| | | | | TTTGGTCTTAAT |
| | | | | CCATGTACACTTTTTTATTTCTGTATTTTTCCACTTCACTGTAAAAATAGTATGTG |
| | | | | TACATAATGTTT |
| | | | | TATTGGCATAGTCTATGGAGAAGTGCAGAAACTTCAGAACATGTGTATGTATTA |
| | | | | TTTGGACTATG |
| | | | | GATTCAGGTTTTTTGCATGTTTATATCTTTCGTTATGGATAAAGTATTTACAAAA |
| | | | | CAGTGACATTTG |
| | | | | ATTCAATTGTTGAGCTGTAGTTAGAATACTCAATTTTTAATTTTTTTAATTTTTTT |
| | | | | ATTTTTTATTTTC |
| | | | | TTTTTGGTTTGGGGAGGGAGAAAAGTTCTTAGCACAAATGTTTTACATAATTTG |
| | | | | TACCAAAAAAAA |
| | | | | AAAAAAAGGAAAGGAAAGAAAGGGGTGGCCTGACACTGGTGGCACTACTAAG |
| | | | | TGTGTGTTTTTT |
| | | | | TAAAAAAAAAATGGAAAAAAAAAAGCTTTTAAACTGGAGAGACTTCTGACAA |
| | | | | CAGCTTTGCCTCT |
| | | | | GTATTGTGTACCAGAATATAAATGATACACCTCTGACCCCAGCGTTCTGAATA |
| | | | | AAATGCTAATTTT GGA |
| 1000 | CDH2 | HSX2_02495389 | 724 | GAGTCAGCAGAAGTTGAAGAAATAGTGTTCCCAAGACAATTCAGTAAGCACAG |
| | | | | TGGCCACCTAC |
| | | | | AAAGGCAGAAGAGAGACTGGGTCATCCCTCCAATCAACTTGCCAGAAAACTCC |
| | | | | AGGGGACCTTTT CCTCAAGAGCTTGTCAGG |
| 1000 | CDH2 | HSX2_02495398 | 725 | TCCTTGTGCTGATGTTTGTGGTATGGATGAAACGCCGGGATAAAGAACGCCAG |
| | | | | GCCAAACAACTT |
| | | | | TTAATTGATCCAGAAGATGATGTAAGAGATAATATTTTAAAATATGATGAAGAA |
| | | | | GGTGGAGGAGA AGAAGACCAG |
| 11123 | RCAN3 | HSX2_01550572 | 726 | GAGAGAAATATGAACTTCACGCGGGAACAGAGTCGACACCCAGCGTGGTGGT |
| | | | | TCATGTCTGTGA |
| | | | | AAGTGAAACTGAAGAGGAAGAAGAGACAAAAAACCCCAAACAGAAAATTGCC |
| | | | | CAGACGAGGCG |
| | | | | CCCCGACCCTCCGACCGCAGCGTTGAATGAGCCCCAGACCTTTGATTGCGCGCT |
| | | | | GTGAGGCCCTT |
| | | | | GGTTGTGGTGCGAGGCGGCTGCCCTGGTGGGCTCTGGCCATGGCGCTCTGTGC |
| | | | | CTGCGGCCGAT |
| | | | | GCGTTGCTGCGAACAGCATAGGTGAGACTCTGCCGAGTGAGGTATAGGTCTTC |
| | | | | TCACCACGCCTG |
| | | | | TACTGCAGACACGGTCGTGTAGAGTAGCAGCTGATTTGACCTGTCCCAGATTTT |
| | | | | AAGTGATATTCC |
| | | | | AAAAGGGACTTTACATTAAAGGAGAAGCCCCCAAGATGTGGCCACCCTTAACC |
| | | | | ATTTTTAAATAG |
| | | | | TAACAAATTAGGAAAACAGCTCCCCTCCCCTCCAGCCATGTAAGTCCTCCTGATT |
| | | | | CTGTATCACAT |
| | | | | GAGACACCAAAAACTGGAAATGTAGTCACACCCAGTACAGTACCATTTTTATGA |
| | | | | ATTTAAAAATA |
| | | | | GTCTTATAATTTTAATTGTTTGTGGGTATTTTTCTACTGGTATAAATAGCTTCTAA |
| | | | | TTAATAAATCGA |
| | | | | TCAAAGGTTGTTACTCAGTATACATGAAATTTTTTGCAGGAAAAGAAACCAGAA |
| | | | | ATACAGAATAA |
| | | | | TTCATAGAAATCATGGTTCTCAAGTATTTGTTCAATTAGCTTTGGGGAAGAAAT |
| | | | | GCCAAATCCACT |
| | | | | TTGAGGATGAGTCTTACGACACAGATCCACCCACATGTTGCTCTTATCTGGAAT |
| | | | | TTGGGACAA |
| 5981 | RFC1 | HSX2_01182722 | 727 | CAAAGTTTGGTAAATTTTCCGGCAAAGATGATGGCTCTAGTTTTAAAGCAGCGT |
| | | | | TGCTGTCAGGC |
| | | | | CCTCCTGGTGTTGGCAAAACCACCACAGCTTCCCTGGTGTGTCAG |
| 84220 | RGPD5 | HSX2_02090776 | 728 | AGCCTCCATTATGGCATGCTGAATTTACCAAAGAAGAATTGGTTCAGAAGCTCC |
| | | | | GTTCCACCACA |
| | | | | AAAAGTGCAGATCACTTAAACGGCCTGCTTCGGGAAATAGAGGCAACCAATGC |
| | | | | AGTCCTTATGG AGCAAATTAAG |
| 58480 | RHOU | HSX2_01949078 | 729 | GTCGTGAGCCCCTCATCCTTCCAGAACGTCAGTGAGAAATGGGTGCCGGAGAT |
| | | | | TCGATGCCACTG |
| | | | | TCCCAAAGCCCCCATCATCCTAGTTGGAACGCAGTCGGATCTCAGAGAAGATG |
| | | | | TCAAAGTCCTCA |
| | | | | TTGAGTTGGACAAATGCAAAGAAAAGCCAGTGCCTGAAGAGGCGGCTAAGCT |
| | | | | GTGCGCCGAGG |
| | | | | AAATCAAAGCCGCCTCCTACATCGAGTGTTCAGCCTTGACTCAAAAAAACCTCA |
| | | | | AAGAGGTCTTT |
| | | | | GATGCAGCCATCGTCGCTGGCATTCAATACTCGGACACTCAGCAACAGCCAAA |
| | | | | GAAGTCTAAAAG |
| | | | | CAGGACTCCAGATAAAATGAAAAACCTCTCCAAGTCCTGGTGGAAGAAGTACT |
| | | | | GCTGTTTCGTAT |
| | | | | GATGCTGGCAAGACACCCAGAAAGGCTATTTTCAGATGAAATCGATATTAGAA |
| | | | | GCTATATTAGCT |
| | | | | GAAACAACTCCTTTTACTGCGTAGAACCTATATCGAGAGTGTGTGTATATGTAT |
| | | | | TATAGGAGGAG |
| | | | | CTCTCAATTTTATGTATTCTTTCTGCCTTTAATTTTCTTGTTTGTTTGAGCTTAGG |
| | | | | GATGAGATACTT |
| | | | | ATGCAAGATATTTTTGAAGTAAATTAAACATTTTTCACATCTCTGGAAATTTAG |
| | | | | AGTTCTAGACCTC |
| | | | | TGGTTAATTTATATCTAATATGAAGAAGACACCTCTAATCTGGATGTTAAGAAT |
| | | | | GAAGTTCTGCTA |
| | | | | CATTATAATGTACAGAAGAGCAAAAGGGAGGAACACTATGGTTAACCCTCTCT |
| | | | | TGATTAAGGGCT |
| | | | | ACTTAATGCACAGTGCATTATGTACACAGGTCAACCATGGTAACAATAGTTCTT |
| | | | | AGCTTTGAAACT |
| | | | | CCATGCAAACCATGCCTTTTTTTTAAGGAGCAAAAATCTGAGAAAAAAAGTGA |
| | | | | GAGACCTCTGCC |
| | | | | TACAAAACCTCAAACCAGTCACTTTTGTCAATTGCTAATACCCAGTTACTTATGA |
| | | | | TTTAAAAACAAC |
| | | | | CAACAGAAAACATCCCACTGACTGTATGGCACTCTGTAGTCAAAAAAGGAAAC |
| | | | | TTCCTTATTGGG |
| | | | | ACTTTTCTTTCTTAGTCCAGTTGTGTTGACACATATGAACACAGACAAAGTGCT |
| | | | | ATGCGGAGGAAA |
| | | | | GCAAGTGTTGGTCAGTAGTTTCATGTTTTAGGGAGTGGTTCCTGTGGAGATCA |
| | | | | GAAAGTGACATT |
| | | | | TGCTTTCGGTACTGTAATACGTGCACCAAACTGCCTCAATCCTAGGTAACGAGG |
| | | | | GCAACAGGGAG |
| | | | | CACCTGTCTGGATTGTTTTTAAACCTCCATACTCAAGCTGTCTCTTCGGCAGGG |
| | | | | AGGTGAATACTC |
| | | | | TTGAAAGGCCAACAGCAAGTGTTTGTGGGACACAACACAGATAATTTTTTCTTA |
| | | | | AGTCGGCCAAG |
| | | | | ATGTACTTCTCTGTGTGCACACCCATGCACACTCATGCACACAGATACATAGGT |
| | | | | CTGTATGGCTGT |
| | | | | ATTTGCTGTTGATTCAGACTTTCACACCATTAATGGGGAAAAGCGTGGCCACA |
| | | | | AAAACAGATGCT |
| | | | | AGGAAGCTTGGCTTCCTCTTCTTGTTGACCCTTTTTTGAACCAACATCTTTTTTAT |
| | | | | TATATTCAGAGT |
| | | | | ATGTTTTTAAGTGTATCTTAATATATACATTTTTTAGGACATCTTAAATCTAAAC |
| | | | | AAAAAATAAAAT |
| | | | | GAACATCTCTTGAAACCTGTTAAAACAACCAGTTAAAGCCACAGATGGCTTTCA |
| | | | | GGGCAGTAGCA |
| | | | | GCAGAGGCCAGTGGACTCTGAGGACTCCTGAGGGGCGGGGCGTGTAGCCAG |
| | | | | CCAGGTGCATGC |
| | | | | CGGGACCATGGCCCCCATACTTGGCTGCTTCCTGTGACAGTGAAATACATCCTT |
| | | | | CAAGGTGGCAG |
| | | | | CTGTTAGGGCTGAATCTTCTGGAGAAAAAGGTGCCATCTCAGGAGAATAGCTT |
| | | | | TTACTCTGGTAG |
| | | | | GAATGCTTCCGAGACACCACAAGGCAGCCTGAACACTCAGTTGCAGGGTCGG |
| | | | | GCTTGCGGTGGG |
| | | | | TGACCCAGAGCCACCAAAGTCACATCCACAACTAATGAGGGAAATCTGTAAAG |
| | | | | CCAGTTAGATAG |
| | | | | AAGAATTTTATTTTTCTGTGGGTTTTGTGTTGTCTTTTTTATGTTAAAAAGAAAT |
| | | | | CCAGTTTGTGTTT |
| | | | | TTCTATAGAAAAAGTAAAAGATCAGGTTATACTTTAGGTTAGGGGTTCTATTTA |
| | | | | TTCCTGTTAGTA |
| | | | | AATAAAATTAACAAATTTCTTTGTTTAACAAAAGATTAATCTTTAAACCACTAAA |
| | | | | ATACATAGACTG |
| 63891 | RNF123 | HSX2_01961187 | 730 | CCCACCAGTGAGGAGGACCTCTGCCCCATCTGCTATGCCCACCCCATCTCTGCT |
| | | | | GTGTTCCAGCCC TGTGGCCACAAGTCCTGCAA |
| 81847 | RNF146 | HSX2_02066510 | 731 | GCCGCAACGTACTCCGGGTCGGCCTTGCGCTCGG |
| 6569 | SLC34A1 | HSX2_01226407 | 732 | TGCTGGAGGTGAGCTCTGCCATCCCCATCATCATGGGCTCCAACATCGGCACC |
| | | | | TCTGTCACCAAC |
| | | | | ACCATCGTGGCCCTGATGCAGGCGGGGGACAGGACTGACTTCCGGCG |
| 6258 | RXRG | HSX2_01203661 | 733 | CTCCGATCTAGAGGCAGATTCCTGACTAATCCCAGAGGGCTGGCCCAGCCTGT |
| | | | | GCTCCCCGGGCT |
| | | | | GCTAGGAAGCGATGACCACTCTTGTTAGCCCAAGTTGAAGAAAGCCGGGCTGT |
| | | | | GCCTGGGAGCC |
| | | | | GAGAGAGGCGGTAATATTTAGAAGCTGCACAGGAGAGGAACATGAACTGAC |
| | | | | GAGTAAACATGT |
| | | | | ATGGAAATTATTCTCACTTCATGAAGTTTCCCGCAGGCTATGGAG |
| 6258 | RXRG | HSX2_01203664 | 734 | GCTCCCCTGGCCACACTGGCTCTACATCCATGAGCCCATCAGCAGCCTTGTCCA |
| | | | | CAGGGAAGCCA |
| | | | | ATGGACAGCCACCCCAGCTACACAGATACCCCAGTGAGTGCCCCACGGACTCT |
| | | | | GAGTGCAGTGG |
| | | | | GGACCCCCCTCAATGCCCTGGGCTCTCCATATCGAGTCATCACCTCTGCCATGG |
| | | | | GCCCACCCTCAG |
| | | | | GAGCACTTGCAGCGCCTCCAGGAATCAACTTGGTTGCCCCACCCAGCTCTCAG |
| 6258 | RXRG | HSX2_01203667 | 735 | TCCCGCAGGCTATGGAGGCTCCCCTGGCCACACT |
| 6258 | RXRG | HSX2_01203684 | 736 | GTTACCAACATATGTCATGCTGCTGACAAGCAGCTTTTCACCCTCGTTGAATGG |
| | | | | GCCAAGCGTATT |
| | | | | CCCCACTTCTCTGACCTCACCTTGGAGGACCAGGTCATTTTGCTTCGGGCAG |
| 6258 | RXRG | HSX2_01203703 | 737 | TGTACTCTTTAACCCAGATGCCAAGGGCCTGTCC |
| 6258 | RXRG | HSX2_02517790 | 738 | ACAAATGACCCTGTTACCAACATATGTCATGCTGCTGACAAGCAGCTTTTCACC |
| | | | | CTCGTTGAATGG |
| | | | | GCCAAGCGTATTCCCCACTTCTCTGACCTCACCTTGGAGGACCAGGTCATTTTG |
| | | | | CTTCGGGCAG |
| 6284 | S100A13 | HSX2_01205966 | 739 | CCTGACAAAGGTCAGCTAGCCCCTTGAGGACATCAGCTTTGGCCTCAGGGTCC |
| | | | | TAATGGCAGCAG |
| | | | | AACCACTGACAGAGCTAGAGGAGTCCATTGAGACCGTGGTCACCACCTTCTTC |
| | | | | ACCTTTGCAAGG |
| | | | | CAGGAGGGCCGGAAGGATAGCCTCAGCGTCAACGAGTTCAAAGAGCTGGTTA |
| | | | | CCCAGCAGTTGC CCCATCTG |
| 6284 | S100A13 | HSX2_01205987 | 740 | AATCGACCACGGAAATTTGACACCTCCGGGCTTGGAAGCAGCTCTCTCCTCCTT |
| | | | | CCCCGCTGCTTA |
| | | | | TAAACCTCAGCCCTGAGGCTCCAGCTCACTCTACCCCATCTCCTTGCCGG |
| 6284 | S100A13 | HSX2_01205990 | 741 | CCCCATCTCCTTGCCGGGTCAGCCCTGACAAAGG |
| 6284 | S100A13 | HSX2_02517894 | 742 | CCTGACAAAGGTCAGCTAGCCCCTTGAGGACATCAGCTTTGGCCTCAGGGTCC |
| | | | | TAATGGCAGCAG |
| | | | | AACCACTGACAGAGCTAGAGGAGTCCATTGAGACCGTGGTCACCACCTTCTTC |
| | | | | ACCTTTGCAAGG |
| | | | | CAGGAGGGCCGGAAGGATAGCCTCAGCGTCAACGAGTTCAAAGAGCTGGTTA |
| | | | | CCCAGCAGTTGC CCCATCTGCTCAAG |
| 6299 | SALL1 | HSX2_01206855 | 743 | TTATTCTGCCCCAGCTGATGTTTGAGCCAGC |
| 6299 | SALL1 | HSX2_01206862 | 744 | CAGATCAAGTGGACTGCAGCGACCTTTCAGAACACAACGGACTTGACAGGGA |
| | | | | AGAGTCCATGGA |
| | | | | GGTGGAGGCCCCGGTTGCTAACAAAAGCGGCAGCGGCACTTCCA |
| 6299 | SALL1 | HSX2_01206865 | 745 | TGACACAGTTAACAAAACAGATCAAGTGGACTGC |
| 6297 | SALL2 | HSX2_01206829 | 746 | AATTCCTGCCCCATCTGCCAGAAGAAGTTCACCA |
| 401474 | SAMD12 | HSX2_02398686 | 747 | ACATGGCCTGTTCCTCACAGAAAGACTAACCCATGGCACTGCCAGAAGATATC |
| | | | | CTTGGATCGTGG |
| | | | | ACAGATTAACTGGATTAACTGAAAAAAGCACCCATTCAGTCAATATTATGGGTT |
| | | | | TTGGAAAAATCT |
| | | | | TGCTTTGCTGTGGATTTAAAATGAGGTGCATAATATAATGCCAACTCATTCCAA |
| | | | | GTGGTCATTGGC |
| | | | | AGACCATTAGCTGGAAAATTGCTTCCAGTTTTCATCACTGTGCATAACTCAAGT |
| | | | | TCCCTTTGGGGA |
| | | | | GCTGTCTGTGCCCTTCACTAGAAGTCCTGTTTTAGCATGGCAACTACTGGATTA |
| | | | | TTTTATTATGTGT |
| | | | | GCAGATGGCATGATGTTTTAGAAAAAAAAAATTACAGCAATTCTGCAATCTCA |
| | | | | GTGGAAGTGTTC |
| | | | | ACAATATTATGCCCTGGTAGGAATGTAAACACGTCTCGATTATGTTAGTTATGA |
| | | | | TGTCTAAAGGTA |
| | | | | TACTGTCCAACTCTTAAGCACTTTATATGAGAACACTACACCAAGATGAATCAG |
| | | | | CACATTCAGCAC |
| | | | | ATGTGAGAAATCAAGTATTTTCTTTTTATTTTTATAAATCTTAGTGGTTTTTTTTT |
| | | | | TTCCGGCTCAGA |
| | | | | CTGTCCAAGACAAACTCCACTACTCGCATATCCCCAGTTGTTTGTGGTAGGTTG |
| | | | | ATGTAAATATGG |
| | | | | CCACAATACTGTTAGACTTCACCTCCAGATACTTTATTGAACAACTGGTTTACTT |
| | | | | CTAGCACCAAAG |
| | | | | GGAAGAAGAAGGGGGTCCAGTGATGGTGATATCTTAGCTTGCTTCTTTGTTCA |
| | | | | CAGGCACTATTG |
| | | | | ACCACATGAACATACAGGTACTCAAATACAGGAGGCTTCTATTTGGCTCCTCCA |
| | | | | AGGGAGGAGTT |
| | | | | CCTCCAAGGGAAAAAAGGGGATGGTATTCTTTTAATCAAAGACCATGGTGGAT |
| | | | | GGATGGTAAGG |
| | | | | GCAACAGAATGCAATTCTTTTGTGTGGTCTGACGTTAACACTTCGGAAAAAATT |
| | | | | CTAAGTCAAGTT |
| | | | | GAAATCGCCATTCTCTCAAGAGAGAAACATCAAGAGAGAAACATCAAGTGACC |
| | | | | CCACTATGTGCC |
| | | | | TAGAGAAGGAAAAGGGTCAAGTCCTATACTAGGTTCCTTACTTAGGGTAGAAC |
| | | | | CTACTAGTCCCT |
| | | | | TGTTAATCCTTTCTGGGATTATACCTGTGACTAATTTTTTTTTTTTTAACTATGTC |
| | | | | TTACGACAAAGC |
| | | | | ACAGTCCTGGGAAACCAGTCAACTTAATTATTGCTTGTCTTTGAGCATTAGAGA |
| | | | | CATGTTCATATT |
| | | | | GACTTGCTACATAACACTGTAGCATGTTTTTTCCTGAAAATATCAAGTTGGTTG |
| | | | | AATGGAAAGCTC |
| | | | | AGCAAAGTATGTTCTGTGTGCCCACTGTCCCAGCCTGGGACTGACGGGCATCA |
| | | | | GAGGCTTTGCTT |
| | | | | TAAAATATGGGTAACCAGTTCTGGAAGACTTCCAACGCAAGCAAGCTTGGTGT |
| | | | | TTTAGGGTGTCT |
| | | | | CCAGATACATAAGCATTGTCTGCCTCCGCTCAGGGTCTCAGGAAATACACAGC |
| | | | | CCATTGGAAACC |
| | | | | ACCCACGCCTGAGATTTGGAAACCTGACGGAAAGGGAGGAAGGTCCAAATGA |
| | | | | GTCAGCAGCAG |
| | | | | GATGACTCACCACAAGCCTGGCAGGCTGCAGAGCCAGAGAAGGGGGCTTCCA |
| | | | | ACAAGGAACTC |
| | | | | AGAGAAGTTGCAGTCACCACCAAGATCTGGCCCTTCGGCAGGTCAGCACCAG |
| | | | | GACTGCCAGGCC |
| | | | | CTCTGCAGACCGTAATTCTCTTCACATCTATAATTAGGAGGCCAAACTCCAGCT |
| | | | | GCCAACACCCTT |
| | | | | GGACACAATATTCCAGTCTCCACTGCCCATCTCATGTGGTTCAGTTCTAATAGC |
| | | | | AGGTCTTCTTCTT |
| | | | | CCTTTCATTCCTTCCTTTATTCAGCAAGCATTTGTTGAGCATCTACTATGTGCCA |
| | | | | AGCATTGTGCTT |
| | | | | GGGCAACAACAGTCCACAAAGCAGACTCCCCACCTGCCCTTATGAAACTGTAT |
| | | | | CTCCTCCTCCCCA |
| | | | | ACATTATCACCACCCCACCATTTCCACAATGAAAACTGAATTCAGACAGCATTC |
| | | | | TCCATTCTGCCA |
| | | | | GAGTAACCTAGGAAGAAGAACATGGCAGCAGACGGATCCTCAGGGAGGAGG |
| | | | | CAGCAGAGTGAC |
| | | | | AGATGGTAAACTGCATCAGGCCACATATTTGCACCTTTCAGATACAGCCAACTC |
| | | | | TTCCAGGGCCTA |
| | | | | GACTGAGCTGGTTGGAGCTCCCCAAAGCAGCATTCAGTTGCTTTTCCTTGATAT |
| | | | | CCACCCTGGCTA |
| 6447 | SCG5 | HSX2_01215772 | 748 | AGTGTCAATCCATATCTACAAGGACAGAGACTGGATAATGTTGTTGCAAAGAA |
| | | | | GTCTGTCCCCCA |
| | | | | TTTTTCAGATGAGGATAAGGATCCAGAGTAAAGAGAAGATGCTAGACGAAAA |
| | | | | CCCACATTACCTG |
| | | | | TTAGGCCTCAGCATGGCTTATGTGCACGTGTAAATGGAGTCCCTGTGAATGAC |
| | | | | AGCATGTTTCTTA |
| | | | | CATAGATAATTATGGATACAAAGCAGCTGTATGTAGATAGTGTATTGTCTTCAC |
| | | | | ACCGATGATTCT |
| | | | | GCTTTTTGCTAAATTAGAATAAGAGCTTTTTTGTTTCTTGGGTTTTTAAAATGTG |
| | | | | AATCTGCAATGA |
| | | | | TCATAAAAATTAAAATGTGAATGTCAACAATAAAAAGCAAGACTATGAAAGGC |
| | | | | TCAGATTTCTTG |
| | | | | CAGTTTAAAATGGTGTCTGAGGTTGTACTATTTTGGCCAAGTCTGTAGAAAGCT |
| | | | | GTCATTTGATTT |
| | | | | TGATTATGTAGTTCATCCAGCCCTTGGGCATTGTTATACACCAGTAAAGAAGGC |
| | | | | TGTACTCAAGA |
| | | | | GGAGGAGCTGACACATTTCACTTGGCTGCGTCTTAATAAACATGAATGCAAGC |
| | | | | A |
| 51540 | SCLY | HSX2_01771392 | 749 | GCCGTCCCCAGTGCTGCTGAGCTACGGTGTCCCCTTCGACGTGGCCAGGAACG |
| | | | | CGCTCCGGCTC |
| | | | | AGCGTGGGCCGCAGCACCACCAGGGCCGAGGTGGACCTCGTCGTGCAGGACC |
| | | | | TGAAGCAGGCC |
| | | | | GTGGCGCAGCTGGAGGACCAGGCCTAGCACTGGGGCCGCCTTCCCCACCCCG |
| | | | | CTTCTGGGAAGC |
| | | | | CCGTGGCAGGGCACAGGGTTGTCCCTCCAGTTCCCTCCTGAGGGCTGTGCCAG |
| | | | | GATGACTGTCTC |
| | | | | ATGCCCCCTCTGCATTTTGTCCTGGAGTGCCAGCGAGTGTGCACCCCCAGTTTC |
| | | | | CTTCCCTGGACC |
| | | | | CCTGCAGAGCTCACAGGGCCCAGGACACCAACGCCGCATAGGACTGCCCACAT |
| | | | | GGGACCGCCCA |
| | | | | CATAGGACCGCCCACATAGGACCGCCCACATGGGACCGCCCACATGGGACCG |
| | | | | CCCACATGGGAC |
| | | | | CGCCCACATAGAACCGTCCTCCAGTGGTGAAGCGGAAACACTTAGCTTTATCC |
| | | | | ACCCTCCCCACT |
| | | | | GGGAACTGGGCACGCCTGTTGTGAGTGCCCTTTCCTGGAAGGTGTTTTTATCT |
| | | | | GGAAGATAGAAT |
| | | | | CCAAGTATTTATAACTCATTGTCAGCCAAATGCTATCATGAACGTAGGAAACTT |
| | | | | GATTTTTTTGTTT |
| | | | | TGATCATGGCCTCTACATGCACCTTTCCAGAGTGGTCTCTTCTCAGGCCCTTTTT |
| | | | | AGTCCCTTTCCA |
| | | | | AAGCTGCCCCCACCACCCTGCTCCTCTGGCCTCAGTGCACAGTGGCCCCCAGCC |
| | | | | TCGGCCAGGCC |
| | | | | TGCTCTGCTCAGCGCCCACCGCCCACCACCCCTCCTGCTTCCCCGAGCCTGACC |
| | | | | CTGTTCCGCCCA |
| | | | | CTGGCAATCAGGGCTGCGCACTTCCCTGTCCACGGTCCCCAGGCCTTCCTGTCT |
| | | | | TGTCCCTTTTGA |
| | | | | TCATTATTAACTCAGGGTTTCAGCTCCAACCTCGCTGAGTTGGTGCAGCTCCAG |
| | | | | GTCATTCCTGGG |
| | | | | GTGGGAATCGGATCATCCCTGACTCAGCTTTTACCTTAATTTTATTTGCAGAGG |
| | | | | ATTCTTTTCTCAA |
| | | | | AATGCTCTGGCATTTGGACACACATCACATGTCGATATTTGCATAGGAGTCATT |
| | | | | TTCAGTGGAATA |
| | | | | ACATTTTTAATGTGTGGTTTTACGGTTCAAGGAACTACTTGATGATTTTGAGGA |
| | | | | AACACTTGCCAGAAACTAAATTAACGAATAAAAGATTTCAGTGCCCGA |
| 6385 | SDC4 | HSX2_01211518 | 750 | ACTCGCCGCAGCCTGCGCGCCTTCTCCAGTCCGCGGTGCCATGGCCCCCGCCC |
| | | | | GTCTGTTCGCGC TGCTGCTGTTCTTCGTAGGCGGAGTCGCCGAGTCG |
| 6385 | SDC4 | HSX2_02518216 | 751 | ATCCGAGAGACTGAGGTCATCGACCCCCAGGACCTCCTAGAAGGCCGATACTT |
| | | | | CTCCGGAGCCCT |
| | | | | ACCAGACGATGAGGATGTAGTGGGGCCCGGGCAGGAATCTGATGACTTTGAG |
| | | | | CTGTCTGGCTCT GGAGATCTGG |
| 6385 | SDC4 | HSX2_02518220 | 752 | GTGCCTCTAGATAACCATATCCCTGAGAGGGCAGGGTCTGGGAGCCAAGTCCC |
| | | | | CACCGAACCCA |
| | | | | AGAAACTAGAGGAGAATGAGGTTATCCCCAAGAGAATCTCACCCGTTGAAGA |
| | | | | GAGTGAGGATGT |
| | | | | GTCCAACAAGGTGTCAATGTCCAGCACTGTGCAGGGCAGCAACATCTTTGAGA |
| | | | | GAACGGAGGTC CTGGCAG |
| 89866 | SEC16B | HSX2_02129253 | 753 | CTGGGAAGGGGCTTGTGGAGGCAGCTCACTTCTGCTATCTCATGGCTCACGTG |
| | | | | CCCTTTGGCCAC TACACCGTGAAGACAGACCATCTGGTCTTGCTGGGCAGCAG |
| 728492 | SERF1B | HSX2_02432910 | 754 | CATATCAGATGGACAGATGTATCATTTTGGGGTCCACCATTCAACCCACTACAA |
| | | | | GGAGTTTTTTAA ACAAAAATAGGAAA |
| 26297 | SERGEF | HSX2_01689967 | 755 | CGAGTGGCATCGTGTTCCAGTGGGGGACTGGTTTGGCATCATGTGGACGACG |
| | | | | GTTGTGCCCTGG |
| | | | | GCAGACTCTTCCATTGTTTTTCACAGCAAAGGAACCAAGCAGAGTGACAG |
| 26297 | SERGEF | HSX2_01689996 | 756 | GTGTATGTTTGGGGAAGCAACAAGCATGGGCAACTGGCTAATGAGGCTGCTT |
| | | | | TCCTTCCTGTGCC |
| | | | | CCAGAAAATAGAAGCACATTGTTTCCAGAATGAAAAGGTCACTGCCATCTGGA |
| | | | | GTGGATGGACA CACCTGGTTGCTCAGACAG |
| 5270 | SERPINE2 | HSX2_01123425 | 757 | TTGGTAAAATATTAAAGAAGATCAACAAGGCCATCGTCTCCAAGAAGAATAAA |
| | | | | GACATTGTGACA |
| | | | | GTGGCTAACGCCGTGTTTGTTAAGAATGCCTCTGAAATTGAAGTGCCTTTTGTT |
| | | | | ACAAGGAACAA |
| | | | | AGATGTGTTCCAGTGTGAGGTCCGGAATGTGAACTTTGAGGATCCAGCCTCTG |
| | | | | CCTGTGATTCCA TCAATGCATGGGTTAAAAATGAAACCAGGG |
| 5270 | SERPINE2 | HSX2_01123431 | 758 | ATATGATTGACAATCTGCTGTCCCCAGATCTTATTGATGGTGTGCTCACCAGAC |
| | | | | TGGTCCTCGTCA |
| | | | | ACGCAGTGTATTTCAAGGGTCTGTGGAAATCACGGTTCCAACCCGAGAACACA |
| | | | | AAGAAACGCACT |
| | | | | TTCGTGGCAGCCGACGGGAAATCCTATCAAGTGCCAATGCTGGCCCAGCTCTC |
| | | | | CGTGTTCCGGTGTG |
| 26470 | SEZ6L2 | HSX2_01691108 | 759 | TGACCCAGACCACAGATCCATCACGGCAGCTGGAAGGGGGGAACCTGGCCCT |
| | | | | GGCCATCCTGCT |
| | | | | GCCTCTAGGCTTGGTCATTGTCCTCGGCAGTGGCGTTTACATCTAC |
| 26470 | SEZ6L2 | HSX2_02547311 | 760 | TGACCCAGACCACAGATCCATCACGGCAGCTGGAAGGGGGGAACCTGGCCCT |
| | | | | GGCCATCCTGCT |
| | | | | GCCTCTAGGCTTGGTCATTGTCCTCGGCAGTGGCGTTTACATCTACTACACCAA |
| 253970 | SFTA3 | HSX2_02311072 | 761 | GTTCTTTTTCTTCAGGATCAGGCACAGAGACTGACTGAATGGCTCCAATTATCA |
| | | | | GGATTTGAAAAC CCAGTATCAGAATCTACCACTTTGT |
| 253970 | SFTA3 | HSX2_02311083 | 762 | ATGTTAATTATTCATACTGCATGACTGAGGATTTTGGAGGCAGAGAGAGATTC |
| | | | | ATCTGCAATATTT |
| | | | | GGAACACCAATGGAGGTCTACGTCAACACAGAATTTATACAGCAGCTGGTGCT |
| | | | | AGTCAGAGCTA |
| | | | | ATGACAGAATTTCAGTTTAATAAAAAGACCCCCAACTGAGCACACCATCTTGAA |
| | | | | AAAAGTATACTT |
| | | | | ATCAAACAGCTTTCAATCAGTTCAAGAGAGACACCTTAATTGGGGAGAGGAAG |
| | | | | AATTGCAGAGT |
| | | | | AGTTTGTAATCATGCCAATTCCAGATCAATAACTGCATGTCTGTTCTTTGGTAG |
| | | | | AAATAGCTTTTGC |
| | | | | TTTATATTAAGTAATCACATATATATTCTCTCTATTTGGATAAGGAAACCTTCGC |
| | | | | TTTATTTGACAAT |
| | | | | GTATAATGATATACTCTTCTAATTCACCTCTGTGTCTTCACAATAAACATGAGTA |
| | | | | AAATTTAGACAA |
| | | | | GTGATGGTAAAGGTCAATATAATTATTTATTTTTAAAATAAATTTTGTATCTAAC |
| | | | | AGGAAAGCAGT |
| | | | | TCTTATGAAATTTTTATATTTTCAAAAATTGTTTTGTTCAAATAAAATTTTATGA |
| | | | | GTAAAGTTAA |
| 166929 | SGMS2 | HSX2_02271509 | 763 | AGATCCGTGGGTTGTGCGTCAGCTTTGGGTGAATTTTGTTTCTACCCTGTCACG |
| | | | | GGGAAAGTTCG |
| | | | | GGTTGAGTCCAGGAGTGCACACTGCTGCTGCCACCCAATGCGCTACATATCAC |
| | | | | TTTTTTTTGTTTT |
| | | | | GTTTTGTTTTGTTTTTAAAAGATCATTTTATCTTAAAAAGGAAAGCTGATCCAAG |
| | | | | TAAACACGAAA |
| | | | | GTATTTGACACACCCCACAGATTTTACATGTGTGTAAATGTTTCACTTTAAAATC |
| | | | | TCTATGACAGAT |
| | | | | ACACAGGAAACATGAGATGGTTTCTGCTAATGAGTGGCCCTTGAGTACACACT |
| | | | | TAGATGCTGTCT |
| | | | | GCCCTGTAAATTTGGATCTGGTGCCCCAGGGCAGTCAACTCTTCTAGCACAGG |
| | | | | CTGAAAACACGT |
| | | | | GTGTGTCAACTGAGGTTCACACCACTTGGGGAATGAGCCTGTTTTCTTTCCAGG |
| | | | | TCAGGCCCTGG |
| | | | | TGTGAGATCAATTTTAGGGCTCCTAATTGGAGCACAGAAATGTATTTGGTCAA |
| | | | | ATTTCATTGAAGG |
| | | | | TGATTTCTTCCTTTTTCTGTTGTACTTTATGGAAAAACCAAGATGGAACCTGAAA |
| | | | | GTTAATGTATCC |
| | | | | TTGCTTTTAGCAAGAGACTCAGTTTCTTATAACTAGTCCTAAGGACATATGCCG |
| | | | | CAATTGAGTAAT |
| | | | | TTTACTTTTACCCTATGAAAATAACATGGTGCTGCACTATAATATACTCTCGGAA |
| | | | | TCAGTGTGTTAG |
| | | | | TTACAGCTATACAGTGAAGGGGGATAACTGTTTCTAAATTTCTTTAAGGTGATG |
| | | | | CCAAAAAATGG |
| | | | | ATTAAAAAATCTGATCAGATTTAATGTTACCAGATTTAGTGCATTATTTAATGCT |
| | | | | ATTGGATCTTTT |
| | | | | GGATAATTCTTGGCTTAATTTCTTAGCTACTTGAAGGTTAATTTGCAAGACTTTT |
| | | | | AAAACCTTAGAA |
| | | | | AAGTTTTAAGGTTGCAAAGTTATCAACACTGGGGCAGAGGGTGGAGAGGCCA |
| | | | | ATGCGGGTAGA |
| | | | | AGGAGGCAGTTATGTTTATATTGAAGGTGAAATTTTTCTTTCATTTAGAATGGA |
| | | | | AAACATCCCCAA |
| | | | | ATGTATCATTATAAACTAGTCAGCCTTGACTACACAAAATTGACCTTTAAGTTG |
| | | | | CTTGAGAAAAAC |
| | | | | ACAATGCAAATCGTTCAGAAGGGTCAACATCCTTTGGTGCTAAAATCTTGTGTA |
| | | | | TGTTTTCAGAAT |
| | | | | GGCTTTTTCTGTATGTTATAGAATAATCACTAAAGGAAAGGTAGTTGAATTTAA |
| | | | | AGTCATGAAGCA |
| | | | | AGACTCTTTAATTCAGTTATTTTAAACAAGAATTAAAACCCCAAACATCCTTGGC |
| | | | | AGGCTTTGAAG |
| | | | | CACACAGAATTTTCTAGTATTTCTTATTAAATACACCAATAATAAACATATACTT |
| | | | | AGTTTACCTGAT |
| | | | | GAGTTTAGACATAATTCTATATAATGTTCGCTTATATTCATTTATTAACTAGCAA |
| | | | | CACTTGTGCAAG |
| | | | | AACAAGGTATTCTGCTTGAAAACACTGATTTTTAAGTTCCTGTTGCCCGATTGT |
| | | | | AAAGAAAATCAT |
| | | | | CTGACACAGAAGCCTGGATTTTGCTCTCCTAACACTGGTGTTTTCCTTGAGCCTC |
| | | | | TAATTCAAATA |
| | | | | AAACAAACTGATACACCTTACAGCCTTATTCATCTGTGTCTGGATAACTCACTTT |
| | | | | TCATCTATTGAA |
| | | | | TAAGAAAAAGTGTTTAAACTTAATAAAATAAAAAATTGTGCACCAATTACTACA |
| | | | | GATCCTTGAAAC |
| | | | | AGTAAGCCTTGGATTTTAGTCAAGTAATCCAGTTTTTTTTTAATTTAAAAAAAAC |
| | | | | CATCACCTTTTA |
| | | | | AAGAACTTTAAAAACACCTGTGATGCCAATGTGGACATTGCCGCTGCCATGTCT |
| | | | | TTACACGCATGT |
| | | | | GTCGTATAGCTCTGTCATCGAGTTGAGGAAGTCCATGGTTTGCAAATAAGGGT |
| | | | | GGGGGAATCAA |
| | | | | AATTTCAAGATGGAAAAATAAAACAAATAGTAAAACCCATTTACATGTAGGAA |
| | | | | AGAAAAGTTGAG |
| | | | | CTTCAACCCAATGTGCCACTTTGATATAAAACAAGTATGATATATATATAAACT |
| | | | | GAAGATATAAAA |
| | | | | CAAGTATGATCTTGCCTTTGAAATCATAAATGTTTGGGGTACCTGTTCTTTGCCA |
| | | | | GTTAAGATACA |
| | | | | TATCTTATTATCTTTGTTTTTTTCAAGTCTATGCTCCTGTTTGAAGCTTTTCCTGT |
| | | | | AATTTAGGTTGTC |
| | | | | TGTGAAATACCTATAACATATAATTCCTATAGAGTATGCCACATTTTTTTTCTAA |
| | | | | CTCATTTCAAAT |
| 6449 | SGTA | HSX2_02518607 | 764 | GAGATGCCGCAGGACCTGAGGAGCCCCGCGCGAACCCCGCCTTCCGAGGAGG |
| | | | | ACTCAGCAGAGGCAGAGCGCCTCAAAACCGAAG |
| 57477 | SHROOM4 | HSX2_01923419 | 765 | TTGGTACTGATAGAGAAGAAGCAGCAGCTGACGGGGCAGTTGGCAGATGCCA |
| | | | | AGGAGCTGAAG |
| | | | | GAGCACGTGGACCGCCGGGAGAAGTTGGTGTTTGGCATGGTCTCCCGCTACCT |
| | | | | GCCTCAGGACC |
| | | | | AGCTCCAAGATTACCAGCACTTTGTCAAGATGAAATCTGCTCTCATCATTGAAC |
| | | | | AGCGAGAGCTG |
| | | | | GAGGAGAAGATCAAGCTCGGGGAAGAGCAACTCAAATGTCTCAGGGAGAGTC |
| | | | | TACTCCTGGGG |
| | | | | CCCAGCAATTTCTAATTCTACCAGCACTCTGCCACAGCATCCCTGCCCAGCCAT |
| | | | | GTGGGAAGTGCT |
| | | | | TTCAATCTTCTTTGTTAGCAGTTTCTCAGCAAGTAGATAGCAATTAGCAGTTTGT |
| | | | | TCCAGCCCTCTA |
| | | | | CCCTGGATGTCTCTCACTACCCCTTCCCTAGCAGTGGTCCTAACCAGCTAGGAG |
| | | | | ACCCTGGGGAA |
| | | | | GCCACAAGCTTCTACCCAAGGGAGCTGCAGCAAGGTGTGATCTTAGAACCACA |
| | | | | CTCTCCTTCCCA |
| | | | | CAGTTGCCAAGGGCAAGTACTTGCTGCACAGAGAACCAAGGAAGTGCCTTCAT |
| | | | | TCTGCTTTGTAC |
| | | | | TAGGACACCAAAGACATCAAGTACTCATCACCCACCCATATCATCAACAGCCTC |
| | | | | TAAAGGCTCAG |
| | | | | AGGGAATCTGCCTTGCAGCTCTACTCTGCCCCAGGGCTTGTGGCCAGCCATTTC |
| | | | | TCACAGAGAGC |
| | | | | TGGCTGCCTTGAGGGCATTCACCTGGCACCAGTTTCAGGGCCTCACCCAAGCTT |
| | | | | TGCAGGGGAAA |
| | | | | GCACAGAGGGAGGAATTACACTGAAAAAAATGCAAGCAAAGGTTGAGTACCC |
| | | | | CCAGGTGCCCCT |
| | | | | TAGGAAGGAACCAGGTTTAAATAGGCTCTACCCTTACCTTTCCCAGCAGCAAGT |
| | | | | TCAGGGGAAGA |
| | | | | GGCCTACTCTTAGCCCTGGCTAGTGTGACCCTCTTCCTGTCCTAAGACTTTGGTC |
| | | | | CTACCACCTCTT |
| | | | | GTTTCATCTTTCCTTTACATTGCTGGGGGTTACCGCAGGTGCCTACCCCAGGGC |
| | | | | TTCACCATATGG GCCATTAATAGCTCTACTAAAACTGACTTCTAGAT |
| 57477 | SHROOM4 | HSX2_01923422 | 766 | GCGAGAAGAGGGAAGTTGGAGAGGGAAAAGTTAGCCCAGAAGGAAAGCATT |
| | | | | TTCTGCAGATCA GCCTGAATCCACCGTGGCTAG |
| 8170 | SLC14A2 | HSX2_01323136 | 767 | ACAAGCACCTTGCCCTCCAGTTCATAGACTGGGTCCTGAGAGGGACCGCTCAG |
| | | | | GTGATGTTCATC |
| | | | | AACAATCCTCTCAGCGGCCTCATCATCTTCATAGGGCTGCTGATCCAGAATCCC |
| | | | | TGGTGGACAATC |
| | | | | ACTGGGGGCCTGGGGACAGTGGTCTCGACCTTAACAGCTCTCGCCTTGGGCCA |
| | | | | AGACAG |
| 8170 | SLC14A2 | HSX2_01323147 | 768 | CCACAGGCCACTACAACCTCTTCTTCCCCACAACACTGGTAGAGCCTGTGTCTT |
| | | | | CAGTGCCCAATA TCACCTGGACAGAGATGGAAATGCCCCTG |
| 8170 | SLC14A2 | HSX2_01323168 | 769 | GTGGGCGTGCCACCAGGCACCTGGGCCTTCTGCCTTGCCACCATCATCTTCCTG |
| | | | | CTCCTGACGAC |
| | | | | AAACAACCCAGCCATCTTCAGACTCCCACTCAGCAAAGTCACCTACCCCGAGGC |
| | | | | CAACCGCATCT |
| | | | | ACTACCTGACAGTGAAAAGCGGTGAAGAAGAGAAGGCCCCCAGCG |
| 8170 | SLC14A2 | HSX2_01323213 | 770 | CACTGTTTGCTGCCTACCTGGGTGCTGCCCTGGCTAACATGTTATCTGTG |
| 8170 | SLC14A2 | HSX2_01323231 | 771 | ACAGGACCTGAATGACCGATTGTTTCTTGCTTAC |
| 9194 | SLC16A7 | HSX2_01390568 | 772 | GTTTAGGTTTAGCCTTCAACCTGCAACCCGCCTTAACCATAATTGGCAAATACTT |
| | | | | CTATAGGAAGC |
| | | | | GACCCATGGCAAATGGATTGGCCATGGCAGGAAGTCCTGTTTTCTTAAGTTCAT |
| | | | | TGGCTCCTTTCA |
| | | | | ATCAGTACCTTTTTAATACTTTTGGCTGGAAAGGAAGCTTCCTGATTTTGGGAA |
| | | | | GTCTACTTTTGA |
| | | | | ATGCCTGTGTGGCTGGTTCCCTCATGAGACCCCTTGGACCCAATCAAACCACTT |
| | | | | CTAAGTCTAAAA |
| | | | | ATAAGACTGGCAAAACAGAAGATGATTCAAGCCCAAAGAAAATCAAAACGAA |
| | | | | GAAATCAACTTG |
| | | | | GGAAAAAGTTAATAAGTATTTAGATTTCTCCCTTTTTAAGCATAGAGGATTTCT |
| | | | | GATATATCTGTCT |
| | | | | GGAAATGTCATTATGTTCCTAGGTTTTTTTGCCCCCATTATATTCTTGGCTCCAT |
| | | | | ATGCTAAAGACC |
| | | | | AAGGAATTGATGAGTACTCGGCAGCTTTTCTGCTATCTGTTATGGCTTTCGTTG |
| | | | | ATATGTTTGCTA |
| | | | | GGCCTTCTGTAGGATTAATTGCAAACTCCAAATATATTCGACCTCGAATTCAGT |
| | | | | ACTTCTTCAGTTT |
| | | | | TGCAATCATGTTCAATGGAGTGTGTCACCTCTTGTGCCCACTGGCACAGGACTA |
| | | | | CACAAGCCTGG |
| | | | | TATTATATGCTGTATTTTTTGGCCTTGGATTTGGGAGTGTTAGCAGTGTTCTCTT |
| | | | | TGAAACTCTCAT |
| | | | | GGACCTCGTGGGTGCACCAAGATTTTCCAGTGCCGTCGGACTTGTCACAATTGT |
| | | | | GGAGTGTGGCC CAGTTCTTCTTGGCCCTCCTCTTGCAG |
| 92014 | SLC25A51 | HSX2_02146193 | 773 | GTTAAAAAAAGTCCTTCATGAAAAAGAAAGATCTTAAGCAGCATGATGGATTC |
| | | | | AGAAGCTCATGA |
| | | | | AAAGAGGCCACCAATACTAACATCTTCAAAACAAGATATATCACCTCATATTAC |
| | | | | AAATGTTGGTGA |
| | | | | GATGAAGCATTACTTGTGTGGCTGCTGTGCAGCCTTCAACAATGTCGCAATCAC |
| | | | | ATTTCCCATTCA |
| | | | | GAAGGTCCTCTTTCGACAACAGCTGTATGGCATCAAAACCCGGGATGCAATAC |
| | | | | TTCAGTTGAGAA |
| | | | | GGGATGGATTTCGAAATTTGTATCGTGGAATCCTTCCCCCATTGATGCAGAAG |
| | | | | ACAACTACGCTT |
| | | | | GCACTTATGTTTGGTCTGTATGAGGATTTATCCTGCCTTCTCCACAAGCATGTCA |
| | | | | GTGCTCCAGAG |
| | | | | TTTGCAACCAGTGGCGTGGCGGCAGTGCTTGCAGGGACAACAGAAGCAATTTT |
| | | | | CACTCCACTGG |
| | | | | AAAGAGTTCAGACATTGCTTCAAGACCACAAGCATCATGACAAATTTACCAACA |
| | | | | CTTACCAGGCTT |
| | | | | TCAAGGCACTGAAATGTCATGGAATTGGAGAGTATTATCGAGGCTTGGTGCCC |
| | | | | ATTCTTTTCCGG |
| | | | | AATGGACTCAGCAATGTCTTGTTTTTCGGCCTTCGAGGTCCCATTAAGGAGCAT |
| | | | | CTGCCTACCGCA |
| | | | | ACGACTCACAGTGCTCATCTGGTCAATGATTTTATCTGTGGAGGTCTATTGGGT |
| | | | | GCCATGTTGGG |
| | | | | ATTCTTGTTTTTTCCAATTAATGTTGTAAAAACTCGCATACAGTCTCAGATTGGT |
| | | | | GGGGAATTTCAG |
| | | | | TCTTTCCCCAAGGTTTTCCAAAAAATCTGGCTGGAACGGGACAGAAAACTGAT |
| | | | | AAATCTTTTCAGA |
| | | | | GGTGCCCATCTGAATTACCATCGGTCCCTCATCTCTTGGGGCATAATCAATGCA |
| | | | | ACTTATGAGTTC |
| | | | | TTGTTAAAGGTTATATGAAAAAACCATCAGTTAAGTGCCATTTATCAACTGAAT |
| | | | | AGACCTTCTAAGAAGAATGCAGTT |
| 28965 | SLC27A6 | HSX2_01713935 | 774 | ATTTGCTTGGAACGGTAGAAGAAATCCTTCCAAGCCTCTCAGAAAATATCAGT |
| | | | | GTTTGGGGGATG |
| | | | | AAAGATTCTGTTCCACAAGGTGTAATTTCACTCAAAGAAAAACTGAGCACCTCA |
| | | | | CCTGATGAGCC |
| | | | | CGTGCCACGCAGCCACCATGTTGTCTCACTCCTCAAGTCTACTTGTCTTTACATT |
| | | | | TTTACCTCTGGA ACAACAG |
| 28965 | SLC27A6 | HSX2_01713976 | 775 | AGAGAAGGAGAAAAGGATCATAAGGTGCGTTTGGCAATTGGAAATGGCATAC |
| | | | | GGAGTGATGTAT |
| | | | | GGAGAGAATTTTTAGACAGATTTGGAAATATAAAGGTGTGTGAACTTTATGCA |
| | | | | GCTACCGAATCA |
| | | | | AGCATATCTTTCATGAACTACACTGGGAGAATTGGAGCAATTGGGAGAACAAA |
| | | | | TTTGTTTTACAA A |
| 28965 | SLC27A6 | HSX2_01714019 | 776 | GAGAACCTGGACTTCTCATTTCTCGAGTGAATGCAAAAAATCCCTTCTTTGGCT |
| | | | | ATGCTGGGCCTT |
| | | | | ATAAGCACACAAAAGACAAATTGCTTTGTGATGTTTTTAAGAAGGGAGATGTT |
| | | | | TACCTTAATACTG |
| | | | | GAGACTTAATAGTCCAGGATCAGGACAATTTCCTTTATTTTTGGGACCGTACTG |
| | | | | GAGACACTTTCA G |
| 28965 | SLC27A6 | HSX2_01714061 | 777 | GTTATGAAGGAAGAGCAGGAATGGCTTCTATTATTTTAAAACCAAATACATCTT |
| | | | | TAGATTTGGAAA |
| | | | | AAGTTTATGAACAAGTTGTAACATTTCTACCAGCTTATGCTTGTCCACGATTTTT |
| | | | | AAGAATTCAG |
| 28965 | SLC27A6 | HSX2_01714088 | 778 | CTTTCATATGCTTTCTTAGGAAGAGTGAGAGGGGGGTATATGATTCTTTATGAA |
| | | | | ATGGGGAAAGG |
| | | | | GAGCTAACATTAATTATGCATGTACTATATTTCCTTAATATGAGAGATAATTTTT |
| | | | | TAATTGCATAAG |
| | | | | AATTTTAATTTCTTTTAATTGATATAAACAGTAGTTGATTATTCTTTTTATCTATT |
| | | | | TGGAGATTCAGT |
| | | | | GCATAACTAAGTATTTTCCTTAATACTAAAGATTTTAAATAATAAATAGTGGCT |
| | | | | AGCGGTTTGGAC |
| | | | | AATCACTAAAAATGTACTTTCTAATAAGTAAAATTTCTAATTTTGAATAAAAGAT |
| | | | | TAAATTTTACTG AAATATTTTA |
| 2542 | SLC37A4 | HSX2_00934722 | 779 | GTAGGGCCTGCTCTTTTCTACCATGGCAGCCC |
| 55652 | SLC48A1 | HSX2_01863202 | 780 | GTATATGATGGGCCTCCCCATGTGGATCCTTAGTGCTGTGGCAGAGCCCTTGTT |
| | | | | ATTGTGCTGGG |
| | | | | ATTTTCCCTCCAGCTCCCGGCCGGAAGCTGGGCTCACGTGGGAGCTCAGTGCC |
| | | | | CTCCTGCTACAG ATCTGTCTCTTCCTTACAATG |
| 9353 | SLIT2 | HSX2_01400643 | 781 | GTCACCAGTCATTTATGGCTCCTTCTTGTAGTGTTTTGCACTGCCCTGCCGCCTG |
| | | | | TACCTGTAGCA |
| | | | | ACAATATCGTAGACTGTCGTGGGAAAGGTCTCACTGAGATCCCCACAAATCTTC |
| | | | | CAGAGACCATC ACAGAAAT |
| 9353 | SLIT2 | HSX2_01400648 | 782 | ACGTTTGGAACAGAACACAATCAAAGTCATCCCTCCTGGAGCTTTCTCACCATA |
| | | | | TAAAAAGCTTAGACGAAT |
| 9122 | SLC16A4 | HSX2_02529005 | 783 | GAGCCCTTATATTATTTGGAGCTATCGCATTGAATTTGGTGCCTTCTAGTATGCT |
| | | | | CTTAAGACCCAT |
| | | | | CCATATCAAAAGTGAGAACAATTCTGGTATTAAAGATAAAGGCAGCAGTTTGT |
| | | | | CTGCACATGGTC |
| | | | | CAGAGGCACATGCAACAGAAACACACTGCCATGAGACAGAAGAGTCTACCATC |
| | | | | AAGGACAGTAC |
| | | | | TACGCAGAAGGCTGGACTACCTAGCAAAAATTTAACAGTCTCACAAAATCAAA |
| | | | | GTGAAGAGTTCT |
| | | | | ACAATGGGCCTAACAGGAACAGACTGTTATTAAAGAGTGATGAAGAAAGTGA |
| | | | | TAAGGTTATTTC |
| | | | | GTGGAGCTGCAAACAACTGTTTGACATTTCTCTCTTTAGAAATCCTTTCTTCTAC |
| | | | | ATATTTACTTGG |
| | | | | TCTTTTCTCCTCAGTCAGTTAGCATACTTCATCCCTACCTTTCACCTGGTAGCCA |
| | | | | GAGCCAAAACAC |
| | | | | TGGGGATTGACATCATGGATGCCTCTTACCTTGTTTCTGTAGCAG |
| 113444 | SMIM12 | HSX2_02575493 | 784 | ATGAGGCCATGAATGAGGTCGGGTCAGCCCAGGC |
| 57154 | SMURF1 | HSX2_01912910 | 785 | AACAAAGAACAACAGTCCAGGGCCAAGTTTACTTTTTGCATACACAGACTGGA |
| | | | | GTTAGCACGTGG CACGACCCCAGGATACCAAG |
| 83891 | SNX25 | HSX2_02570708 | 786 | TCTTGAAGCCGGTAGTGGAGTTACTGAGTAATCCAGATTACATTAACCAAATG |
| | | | | CTGCTTGCCCAG |
| | | | | CTGGCGTACAGAGAGCAAATGAATGAGCATCACAAGAGAGCCTACACCTATG |
| | | | | CCCCCTCTTACGA |
| | | | | GGACTTCATCAAGCTCATTAACAGCAACTCTGATGTGGAGTTCTTGAAGCAACT |
| | | | | AAG |
| 134548 | SOWAHA | HSX2_02214232 | 787 | GCATCGCCCTGCTCTGATCCGCCAGACGCGGAGCCCGGGCCCGGGGCAGCGA |
| | | | | AAGGGCCGCCG |
| | | | | CAGCAGAAGCCCTGTATGCTGCCGGTGCGCTGCGTCCCGGCCCCCGCCACGCT |
| | | | | CCGCCTCCGGG |
| | | | | CGGAGGAGCCCGGCCTGCGCCGGCAGCTGTCGGAGGAGCCGAGCCCGCGGA |
| | | | | GCTCCCCTCTGC |
| | | | | TGCTGAGGCGGCTCTCGGTGGAAGAGTCCGGTCTGGGCCTCGGCCTGGGCCC |
| | | | | GGGCCGCTCCCC |
| | | | | GCACCTGAGGCGCCTGTCGCGCGCCGGCCCGCGTCTGCTGAGCCCTGACGCCG |
| | | | | AGGAGTTGCCC |
| | | | | GCCGCGCCGCCGCCGTCCGCGGTGCCCCTGGAGCCGTCCGAGCACGAGTGGC |
| | | | | TCGTGCGGACT |
| | | | | GCCGGGGGCCGCTGGACCCACCAGCTGCACGGGCTGCTGCTGCGCGACCGCG |
| | | | | GCTTGGCGGCC |
| | | | | AAGCGCGACTTCATGTCTGGCTTCACGGCCCTGCATTGGGCCGCCAAGAGCGG |
| | | | | CGACGGCGAGA |
| | | | | TGGCGCTGCAGCTGGTGGAGGTCGCGCGGCGCAGTGGCGCACCAGTCGACGT |
| | | | | GAACGCACGCT |
| | | | | CCCACGGCGGCTACACGCCGCTGCACCTGGCTGCACTGCACGGCCACGAGGA |
| | | | | CGCTGCCGTGCT |
| | | | | GCTGGTGGTGCGTCTGGGTGCCCAGGTGCACGTGCGTGATCACAGCGGGCGT |
| | | | | CGCGCCTACCAG |
| | | | | TACCTGCGGCCCGGCTCCTCGTACGCGCTGCGCCGCCTTCTTGGCGATCCAGG |
| | | | | CCTGCGAGGCAC |
| | | | | CACGGAGCCAGATGCGACCGGTGGTGGAAGTGGCAGTCTTGCTGCCAGGCGC |
| | | | | CCCGTACAGGT |
| | | | | GGCCGCCACCATCCTCAGTTCCACCACCAGTGCATTTCTGGGCGTCCTGGCTGA |
| | | | | CGACCTCATGC |
| | | | | TCCAGGACCTGGCCCGAGGCTTGAAGAAGTCGAGCTCCTTCAGCAAGTTCTTG |
| | | | | AGCGCCTCGCCC |
| | | | | ATGGCTCCACGTAAAAAGACAAAGATCCGCGGTGGTCTGCCAGCCTTCTCAGA |
| | | | | AATCTCTCGTCG |
| | | | | ACCTACTCCGGGGCCTTTAGCTGGTCTAGTGCCCAGTTTTCCTCCAACAACCTG |
| | | | | AAGGTCCCTGG |
| | | | | GGCTACCACCTGGTTGATCTATCGTGGCCTGCTCGTCGCAGTCCAAACCCGCCC |
| | | | | AAGA |
| 56848 | SPHK2 | HSX2_01898098 | 788 | GTCAGGGCTAAGGCCATGGCCCCGCCCCCACCGCCACTGGCTGCCAGCACCCC |
| | | | | GCTCCTCCATG GCGAGTTT |
| 23514 | SPIDR | HSX2_01645611 | 789 | AATGCTTTGTGTAGGCTATTTCTTTTCATCCTTAACACAAATGGAAGGGAGGTA |
| | | | | TTACTGTAAATT |
| | | | | TGCAGATGGGGAAACTGAAGTCATCAAGGTTAAATAACTTGCTGAATTCCCAC |
| | | | | CACCAGTGGATC |
| | | | | ACAAAGCTGTGTGACACTGGACATGACTTCCCAAAAGAACCCCATCTTGCCATC |
| | | | | CTATGATCTGTA |
| | | | | GATGAAAGTAGGGTTTGCTGATGGCCCTGAGAAAATCTTAACAAACTGGATGA |
| | | | | GGCTCAAAGAC |
| | | | | TTTTCCAGGGATATGGCTCCAGAACTGTGACCTTCCCTTTATGGGTCCAGCATG |
| | | | | ACATCGTCCTGC TGCTCCATATGGCATCAAT |
| 6695 | SPOCK1 | HSX2_01236568 | 790 | AGAATATGAACGGGAGCTGGGACCAAAGGACAAAGAGGGGAAGCTGAGGGT |
| | | | | GCACACCCGAG |
| | | | | CCGTGACAGAGGATGATGAGGATGAGGATGATGACAAAGAGGATGAGGTCG |
| | | | | GGTACATATGGT |
| | | | | AGTGCCCACAAGAAAGAGGACACAAGTTTTGCACAAAATTGCAAGTCACTTCC |
| | | | | TATTCCTGCATTT |
| | | | | GTATCTAAGACTCCAAGGCACCAAGGTCTCTTCTCCATTGTTGCTCTCTATACCC |
| | | | | GACCTAAGGTT |
| | | | | TGGAAGACAACTGCTTGTTCCCAGAGGATTCTGATTTTGCATATGTTTGTATGG |
| | | | | GAGAAAGGGTG |
| | | | | TTGTGTTTTTTTTTTGTTGTTGTTTATTTTTTGGATAGGGAAGTCATTGGCTTAAT |
| | | | | TAGAGCCTCCTT |
| | | | | CCTTTCTGTGAGATTTTTCCAACAAGCATGTGATTTACGTGGAATTCTGACAGT |
| | | | | GCAGGGAGCCCC |
| | | | | AACCCTCTTAAATGTCAAAGACCCTTTTTGATTACCCACACTGGTGGTTATTACA |
| | | | | GCATGGTTCCC |
| | | | | AGCCTTACAGTGTCTAAGTGCTTCTCTTGTGTCCTGTAGATGTTGTGAAAAAGA |
| | | | | AAAAAACAAAA |
| | | | | AATACACCACACTGTACTTTTTCCCCCTGCCCCCGTTACTGCCGGTGATTATTAT |
| | | | | TAAAAATTAGTT |
| | | | | TTTTTCACATCATTATATCTGGCTTCCTATAAACAACAGCCTTAATTCAGTCAAG |
| | | | | ACTCCCTTTGGG |
| | | | | GAATTCATTTTATTAAAAATTGGTGTCTGGATACTTCCCTGTACATGCATAAATA |
| | | | | TGCATGCATGTA |
| | | | | CAGAAAGACTGTATGTGTGTGCCTTGCACACACACCCATACCTCTCAGAAAAA |
| | | | | GTGTTTGGGTAT |
| | | | | CTTAAAAACTCGAAAAACAATGATAAATTTCTCAGCTTGTCCAGACCTGGAACA |
| | | | | AAATTTCTGGAA |
| | | | | TAAGAAATTTGTATTAAAGTCCTTTTTTGCACTAACAGTTGGCTCTTGTAGCCTG |
| | | | | CAGGCTGAGGA |
| | | | | AGTCTCTTCTCTGTGCATCAGCAGAGTTACTGAAAGCCTCTGATTGAGAAAAAA |
| | | | | CCTCCGTCTGCC |
| | | | | TAAATCACTTTTCTCGCAGAAGCCATGCGACTCCCACACGACACGGGCAGCTTC |
| | | | | ACAAGCCATCT |
| | | | | CTTTCATTTCTGCTTGAAGCCCCTTGGCTGCAGCAATCCTGTCTGCCATAGGTTT |
| | | | | CTTCCTTCCTTA |
| | | | | CCTACTCAAGGGCTTTTTCTAAGGCATGCACACATATCTCCTGTTCTCTGAGAG |
| | | | | TACCATGGTGTT |
| | | | | CCTTAAAAGAAGAAAATTTCTAATTCTGAACTCAATGTTTTGCTTTTACTCCCTT |
| | | | | TCTACTGACAAA |
| | | | | TCATGATAAGGGCACAAAAGCTGTACAGATTTTTTTTTTTAACCACTCAATCCC |
| | | | | AAATGGAGGCCT |
| | | | | ACAAAGAACATCGTAATAACACATGGAAGCAAACCCTGGGTTTTTAAGAGCAA |
| | | | | ATTCTGTCCCCC |
| | | | | CCTCACTCCCCCAAGTGACAAGATACTAATGAAGAAAGTTCTTCACCATAGTGT |
| | | | | TTGTTTTAACTA |
| | | | | AACTCATTGGAGTCTAGTTCCAAATTTGGTAGGGTCATCATCTCTACATTCCTTA |
| | | | | GGATTTCTCTCC |
| | | | | CTATCAAGCTGGCCCAGATACAAGTACCAAACAGTAGTCTCTGAAGTTCCCCCA |
| | | | | TTTCCTTCAGTA |
| | | | | CCAGTCTATAAGCTACTGTCCGCCACTGATTTTCATTTATCAGGGTGTCCTAATC |
| | | | | AGAATCAGCCA |
| | | | | CCCAAGCAAGCCTCTCTGGCCCACATATCTATCTCTTGCCTTCCCCCATGAACTT |
| | | | | CAGCCTGTCCAC |
| | | | | ACAAAAGCCACATAAACTCAAGCAAGAAATATGTTCAGCCAAAACATGATTAT |
| | | | | AGTGGCAGCTGA |
| | | | | CCAATCCCCCATCCCAAATTGACCATTTAGATGTACCAACTCACCTTAAATTAGC |
| | | | | ATGTTCCAATCC |
| | | | | AGTCGGCATTGCCTGAATACAGTAGCATCATACCTATAGTTGGTCTTAGATAAG |
| | | | | AAATGAACTACT |
| | | | | TGATATAGCAAAGTCCTTTGGCTTCGTAAATAACCCTGAGGTTTTGTACTTACTT |
| | | | | TCCCCATAGGA |
| | | | | AGACAGACCATAGGCAAACTCTGTTTTGGGATCTCAACTCCATCACCTTTGTTT |
| | | | | CAATATTTTTTTT |
| | | | | CTCTCTTGAACAAAACTGAGATAATTTAGAAAACAGGTGCTTAATTGCAATAAA |
| | | | | ATTACTATGAAG |
| | | | | TATATTAAAAATCACGACATTGTAAAATCTCACTTTAGATCATCAAAGAAAACC |
| | | | | ATTGTTACTATCT |
| 56928 | SPPL2B | HSX2_02560330 | 791 | GATCCCCAACAACAGCCTGCCCTACTTCCACAAGCGCCCGCAGGCCCGTATGCT |
| | | | | GCTCCTGGCGC |
| | | | | TCTTCTGCGTGGCCGTCAGCGTGGTGTGGGGCGTCTTCCGCAACGAGGACCA |
| 6709 | SPTAN1 | HSX2_01237303 | 792 | CAACAAGTGGCCCCCACGGATGATGAGACTGGGAAGGAGCTGGTCTTGGCT |
| | | | | CTCTACGACTATC |
| | | | | AGGAGAAGAGTCCCCGAGAGGTCACCATGAAGAAGGGAGATATCCTTACCT |
| | | | | TACTCAACAGCAC CA |
| 6709 | SPTAN1 | HSX2_01237310 | 793 | GATTGGTGGAAAGTGGAAGTGAACGATCGTCAGGGTTTTGTGCCGGCTGCG |
| | | | | TACGTGAAGAAAT |
| | | | | TGGACCCCGCCCAGTCAGCCTCCCGGGAGAATCTCCTGGAGGAGCAAGGCA |
| | | | | GCATAGCACTGCGGCAGGAGCAGATTGACAATCA |
| 6709 | SPTAN1 | HSX2_01237518 | 794 | GAGCAGATTGACAATCAGACACGCATAACTAAGG |
| 6715 | SRD5A1 | HSX2_01238416 | 795 | GCGCGCAATCGTCAGACGAACTCAGTGTACGGCCGCCACGCGCTGCCCAGC |
| | | | | CACAGGCTCCGAG TGCCGGCGCGGGCCGCCTGGGTGGTGCAG |
| 23635 | SSBP2 | HSX2_01650778 | 796 | AATTCTCCCAATAATATGAGCCTGAGTAATCAACCGGGCACTCCAAGGGATG |
| | | | | ATGGCGAAATGG GGGGAAATTTCTTAAATCCTTTTCAGAGTGAGAGT |
| 81849 | ST6GALNAC5 | HSX2_02066372 | 797 | GGATCCCAATCACCCTTCAGTACCTTATCATTATTATGAACCTTTTGGACCTG |
| | | | | ATGAATGTACAATG |
| | | | | TACCTCTCCCATGAGCGAGGACGCAAGGGCAGTCATCACCGCTTTATCACAG |
| | | | | AGAAACGAGTCTT |
| | | | | TAAGAACTGGGCACGGACATTCAATATTCACTTTTTTCAACCAGACTGGAAA |
| | | | | CCAGAATCACTTGC |
| | | | | TATAAATCATCCTGAGAATAAACCTGTGTTCTAAGGAATGAGCATGCCAGAC |
| | | | | TGTAATCCCAGGT |
| | | | | ATTCACTGCATCAGACACCGAGACACTGAACTTCCTGAGCCACCAGACAGGA |
| | | | | AAGGGTAGCAGA |
| | | | | AAACAGCTTCACTCCTCAGGAAGTACCATGGACAGACGCCTACCAGGGGTG |
| | | | | ACAAAGCAGTGCA |
| | | | | GTTGGATTGTAAGGAAAAATTCCGGAATTAATGCATCCTAATGAATGTTGTC |
| | | | | CCCTTCAATGGTGT |
| | | | | TACCTTAGGAGCTGAACATTCAATTCAGTTACACCACTATGACTAAAAACAG |
| | | | | TTTGGATCTCTTAG |
| | | | | TATTGCCTTTGAAACTGCAACATAAGCAACTCAACAATATTAGTTGCATTCCT |
| | | | | TTATAGACATACCA |
| | | | | TGTCAAAGACGTTTTTCTATCAAGTTGTATTCTTTCCTGTTCTATAACCTTTGT |
| | | | | CATCTGTTAGACTC |
| | | | | TGTATGTGTGATTTGTAAAAAGCAGGCTGAAACTATGGACATGATTTCTGAA |
| | | | | GAGCACATCTCCA |
| | | | | CTGACTTTCATAAAGCAAATGTCCAATATTTATTTATTGAGAGTTTTTTAGTG |
| | | | | CAATCTGGGCCAGT |
| | | | | ATTTTTATAGATTATGATTATGTGGTAATTTATCCTTCCTAACTCTTTAATCCT |
| | | | | GAATGATGGTTGG |
| | | | | AAATGGCCTAGAATTAGGTTACTCTGTTCACAATGCTCATTGTTAGCATGCAA |
| | | | | TTGGTATTTGACT |
| | | | | TGGAAGTGTTGTGTTGTATTTTTTGAACCCCTAGGCTTCAGGAAAACTGCTCT |
| | | | | TTTGTAAAAAGAA |
| | | | | TAGCGATGACATTTTCTAATGTGCAGAAATGTTCCAAAAGGACAAAATTGAA |
| | | | | AACCAAAAACTAT GTTATTAAAACAAAAAAATGCTAA |
| 90627 | STARD13 | HSX2_02136138 | 798 | AAATTGAGGCAAAAGAAGCATGTGACTGGCTCCGTGCTGCCGGGTTCCCGC |
| | | | | AATACGCTCAGTTA TATGAGG |
| 202374 | STK32A | HSX2_02290257 | 799 | TCAACTTTGACCACTTTGAAATTTTGCGAGCCATTGGGAAAGGCAGTTTTGG |
| | | | | GAAG |
| 202374 | STK32A | HSX2_02290318 | 800 | CACCTGAGATGTTCAGCTCCAGAAAAGGAGCAGGCTATTCCTTTGCTGTTGA |
| | | | | CTGGTGGTCCCTG GGAGTGACGGCATATGAACTGCTGAGAGGCCGG |
| 202374 | STK32A | HSX2_02290333 | 801 | AGACCGTATCATATTCGCTCCAGTACTTCCAGCAAGGAAATTGTACACACGT |
| | | | | TTGAGACGACTGTT |
| | | | | GTAACTTACCCTTCTGCCTGGTCACAGGAAATGGTGTCACTTCTTAAAAAG |
| 202374 | STK32A | HSX2_02290361 | 802 | TGTCACTTCTTAAAAAGCTACTCGAACCTAATCC |
| 202374 | STK32A | HSX2_02580836 | 803 | AAAGGCAGGCTGAATTGTGATCCTACCTTTGAACTTGAGGAAATGATTTTGG |
| | | | | AGTCCAAACCTCT |
| | | | | ACATAAGAAAAAAAAGCGTCTGGCAAAGAAGGAGAAGGATATGAGGAAAT |
| | | | | GCGATTCTTCTCAG |
| 6812 | STXBP1 | HSX2_01245920 | 804 | TTGTGGAAGATATCAATAAGCGCAGAGAGCCGCTCCCCAGCCTGGAGGCTG |
| | | | | TGTATCTCATCACT CCATCCGAGAAG |
| 6812 | STXBP1 | HSX2_01245932 | 805 | CTTGTCCAGATGCCCTGTTTAATGAACTGGTAAAATCCCGAGCAGCCAAAGT |
| | | | | CATCAAAACTCTGACGGAAATCAATATTGCATTTCTCCCGTATGAATCCCAG |
| 6812 | STXBP1 | HSX2_01245999 | 806 | CCTGAATGAGATGCGCTGCGCCTACGAGGTGACCCAGGCCAACGGAAAGTG |
| | | | | GGAGGTGCTGAT AG |
| 6812 | STXBP1 | HSX2_02488589 | 807 | GTGGGAGGTGCTGATAGGATCCACACACATCCTC |
| 6812 | STXBP1 | HSX2_02520590 | 808 | GCATCACGGAGGAAAACCTGAACAAACTGATCCAGCACGCCCAGATACCCC |
| | | | | CGGAGGATAGTGA |
| | | | | GATCATCACCAACATGGCTCACCTCGGCGTGCCCATCGTCACCGAT |
| 6812 | STXBP1 | HSX2_02520602 | 809 | CGCCCGCTATGGGCACTGGCATAAGAACAAGGCCCCAGGCGAGTACCGCAG |
| | | | | TGGCCCCCGCCTC |
| | | | | ATCATTTTCATCCTTGGGGGTGTGAGCCTGAATGAGATGCGCTGCGCCTACG |
| | | | | AGGTGACCCAGGC CAACGGAAAGTGGGAGGTGCTGATAG |
| 125206 | SLC5A10 | HSX2_02194590 | 810 | CTTTTGACCAGATCGGTGGTTACGGGCAGCTGGAGGCAGCCTACGCCCAGG |
| | | | | CCATTCCCTCCAG |
| | | | | GACCATTGCCAACACCACCTGCCACCTGCCACGTACAGACGCCATGCACATG |
| | | | | TTTCGAGACCCCC |
| | | | | ACACAGGGGACCTGCCGTGGACCGGGATGACCTTTGGCCTGACCATCATGG |
| | | | | CCACCTGGTACTGGTGCACCGACCAG |
| 23345 | SYNE1 | HSX2_01626487 | 811 | TAACATCATGCAGGCAGCTGTGGTACAATATGAA |
| 10454 | TAB1 | HSX2_01499454 | 812 | GCCTTCGATGTGGTGGAGAGGAGCTTCCTGGAGTCCATTGACGACGCCTTG |
| | | | | GCTGAGAAGGCAA GCCTCCAGTCGCAATTGCCAGAG |
| 9882 | TBC1D4 | HSX2_01449478 | 813 | TGATGGAGAAGGGAGAAAAAGGACCTCATCTACCTGCAGCAATGAGTCCCT |
| | | | | AAGTGTGGGAGGA |
| | | | | ACCTCTGTCACTCCTCGCCGGATCTCCTGGCGGCAGCGCATTTTCCTCAGGGT |
| | | | | TGCTTCTCCCATG AACAAATCTCCCTCAGCAATGCAACAGCAAG |
| 8557 | TCAP | HSX2_01344131 | 814 | GACACCCAGAGACATGAGACCTACCACCAGCAGGGGCAGTGCCAGGTGCTG |
| | | | | GTGCAGCGCTCG |
| | | | | CCCTGGCTGATGATGCGGATGGGCATCCTCGGCCGTGGGCTGCAGGAGTAC |
| | | | | CAGCTGCCCTACC AGCGGGTACTGCCGCTGCCCATCTTCACC |
| 6920 | TCEA3 | HSX2_01254327 | 815 | ACTCCCCTGGACCCCCAAAAGGAGAAAAAGGAGAGGAAAGAGAAAAGGCA |
| | | | | AAGAAGAAGGAA |
| | | | | AAAGGGCTTGAGTGTTCAGACTGGAAGCCAGAAGCAGGCCTTTCTCCACCA |
| | | | | AGGAAAAAACGAG AAGACCCCAAAACCAG |
| 6920 | TCEA3 | HSX2_01254358 | 816 | GAAATGGCCAGTGATGAACTGAGGGAGTTGAGGAATGCCATGACCCAGGA |
| | | | | GGCCATCCGTGAG |
| | | | | CACCAGATGGCCAAGACTGGCGGCACCACCACTGACCTCTTCCAGTGCAGC |
| | | | | AAATGCAAGAAGA AGAACTGCACCTATAACCAG |
| 10178 | TENM1 | HSX2_01477556 | 817 | TGCTTGCATTAAAGGACTTCCTCATCCTTTTTTTCATGAAACTGAGCTTGCTTA |
| | | | | ATCAGAGATGGA |
| | | | | GCAAACTGACTGCAAACCCTACCAGCCTCTACCAAAAGTCAAGCATGAAATG |
| | | | | GATCTAGCTTACA |
| | | | | CCAGTTCTTCTGATGAGAGTGAAGATGGAAGAAAACCAAGACAGTCATACA |
| | | | | ACTCCAGGGAGAC |
| | | | | CCTGCACGAGTATAACCAGGAGCTGAGGATGAATTACAATAGCCAGAGTAG |
| | | | | AAAGAGGAAAGA AGTAGAAAAATCTACTCAAG |
| 10178 | TENM1 | HSX2_01477563 | 818 | GTTTCAAATTCTCTCCTGTTTGTTGTGACATGGAGGCTCAAGCTGGGTCTACT |
| | | | | CAAG |
| 10178 | TENM1 | HSX2_01477568 | 819 | ATGTGCAGAGCAGCCCACACAACCAGTTCACCTTCAGACCCCTCCCACCGCC |
| | | | | ACCTCCGCCTCCTC |
| | | | | ATGCCTGCACCTGTGCCAGGAAGCCACCCCCTGCAGCGGACTCTCTTCAGAG |
| | | | | GAGATCAATGACT |
| | | | | ACCCGCAGCCAGCCCAGCCCAGCTGCTCCAGCTCCCCCAACCAGCACGCAG |
| | | | | GATTCAGTCCATCT |
| | | | | GCATAACAGCTGGGTCCTGAACAGCAACATACCATTGGAGACCAG |
| 10178 | TENM1 | HSX2_01477573 | 820 | GCATTTCCTGTTCAAACATGGATCTGGTTCCTCTGCGATCTTCAGTGCAGCCA |
| | | | | GTCAGAACTACCC |
| | | | | TCTGACATCCAATACCGTGTACTCGCCCCCTCCCAGGCCTCTTCCTCGAAGCA |
| | | | | CCTTTTCCCGACCT |
| | | | | GCCTTTACCTTTAACAAACCTTACAGGTGCTGCAACTGGAAGTGCACAGCAT |
| | | | | TGAGCGCCACTGC |
| | | | | AATCACAGTGACTTTGGCCTTGTTACTAGCCTATGTGATTG |
| 10178 | TENM1 | HSX2_01477585 | 821 | TGTTTCAGAAGGGACGGGCGATAGACACTGGAGAAGTTGACATTGGTGCAC |
| | | | | AGGTCATGCAGAC |
| | | | | CATTCCACCTGGTTTATTCTGGCGTTTCCAGATTACTATCCACCATCCAATATA |
| | | | | TCTGAAGTTCAAT |
| | | | | ATTTCTTTAGCCAAGGACTCTCTGCTGGGAATTTATGGCAGAAGAAACATTC |
| | | | | CACCTACACATACT CAG |
| 10178 | TENM1 | HSX2_01477615 | 822 | CTCACTACTTAGATGCTGTCCGAG |
| 10178 | TENM1 | HSX2_01477625 | 823 | ATGGTTTAACCGACTGTGTGGATCCTGACTGTTGTCAACAAAGCAACTGTTA |
| | | | | TATAAGTCCTCTCT |
| | | | | GCCAGGGCTCACCAGATCCTCTTGACCTCATTCAGCAAAGCCAAACTCTCTTC |
| | | | | TCTCAGCACACTT |
| | | | | CAAGACTTTTTTATGATCGAATCAAATTCCTCATTGGCAAGGACAGTACTCAT |
| | | | | GTCATTCCTCCTGA GGTGTCATTTGACAGCAG |
| 10178 | TENM1 | HSX2_01477630 | 824 | GCGTGCCTGTGTGATTCGAGGCCAAGTGGTGGCCATAGATGGAACTCCTCT |
| | | | | AGTGGGAGTGAAT |
| | | | | GTCAGTTTCTTGCACCACAGTGATTATGGGTTTACCATCAGCCGGCAAGATG |
| | | | | GAAG |
| 10178 | TENM1 | HSX2_01477635 | 825 | CTTTGACCTCGTGGCCATCGGTGGCATCTCTGTCATCTTAATCTTCGACCGAT |
| | | | | CCCCTTTCCTGCCT |
| | | | | GAGAAGAGAACACTCTGGTTGCCTTGGAATCAGTTTATTGTGGTAGAGAAA |
| | | | | GTCACCATGCAGA |
| | | | | GAGTTGTATCAGACCCGCCATCCTGCGATATCTCCAACTTTATCAGCCCAAAC |
| | | | | CCTATTGTGCTTC |
| | | | | CTTCACCGCTCACATCATTTGGAGGGTCCTGTCCAGAGAGGGGAACTATTGT |
| | | | | TCCTGAGCTGCAG |
| 10178 | TENM1 | HSX2_01477640 | 826 | GTTGTACAGGAGGAAATTCCCATTCCCTCCAGCTTTGTGAGGCTGAGTTACC |
| | | | | TGAGCAGCCGCAC |
| | | | | CCCTGGGTATAAAACCCTGCTACGGATCCTTCTGACACATTCAACGATTCCTG |
| | | | | TAGGCATGATAAA |
| | | | | AGTACACCTCACAGTAGCTGTGGAAGGGCGACTCACACAGAAGTGGTTTCC |
| | | | | CGCCGCAATTAATC |
| | | | | TTGTCTACACATTTGCTTGGAACAAGACCGACATCTATGGACAGAAGGTTTG |
| | | | | GGGCCTGGCAGAGGCTTTGG |
| 10178 | TENM1 | HSX2_01477645 | 827 | TATCTGTGGGATATGAATATGAAACGTGCCCTGACTTTATTCTCTGGGAGCA |
| | | | | AAGGACAGTCGTT |
| | | | | TTACAAGGTTTTGAGATGGATGCTTCTAACCTAGGAGGCTGGTCTTTGAATA |
| | | | | AGCATCACATTTTG AATCCTCAAAGTG |
| 10178 | TENM1 | HSX2_01477650 | 828 | GAATCATACATAAAGGGAATGGAGAAAATATGTTCATTTCCCAGCAGCCCCC |
| | | | | AGTCATATCAACC |
| | | | | ATAATGGGTAATGGACACCAAAGGAGTGTAGCCTGCACCAACTGCAATGGC |
| | | | | CCAGCCCACAACA |
| | | | | ACAAACTCTTTGCTCCTGTCGCCTTAGCTTCTGGCCCTGATGGCAGTGTGTAT |
| | | | | GTTGGCGACTTCA |
| | | | | ATTTTGTAAGGAGAATATTTCCCTCGGGAAACTCCGTTAGTATTTTGGAATTA |
| | | | | AG |
| 10178 | TENM1 | HSX2_01477665 | 829 | GCATCACAGTTGATAGGCATGGATTTATTTACTTTGTGGATGGGACTATGAT |
| | | | | TCGCAAAATTGATG |
| | | | | AGAATGCTGTGATCACAACTGTAATCGGCTCAAATGGTCTGACTTCCACACA |
| | | | | ACCACTGAGCTGT GACTCAGGAATGGACATCACTCAG |
| 10178 | TENM1 | HSX2_01477670 | 830 | GTGCGATTAGAGTGGCCAACAGACCTTGCAGTAAATCCTATGGACAATTCAT |
| | | | | TGTATGTCTTGGA |
| | | | | TAACAACATTGTGCTGCAAATTTCTGAGAACAGGCGTGTTCGGATCATCGCA |
| | | | | GGACGCCCCATTC |
| | | | | ACTGCCAGGTGCCAGGCATCGATCATTTCCTGGTCAGCAAGGTAGCAATTCA |
| | | | | CTCCACTCTAGAG |
| | | | | TCAGCGAGGGCCATCAGTGTCTCCCACAGCGGGCTGCTCTTCATAGCTGAAA |
| | | | | CAGACGAGAGGA |
| | | | | AAGTAAACCGCATTCAGCAAGTAACCACCAATGGGGAGATCTACATCATCGC |
| | | | | TGGTGCCCCCACT |
| | | | | GACTGTGACTGCAAAATTGATCCAAACTGTGACTGTTTTTCAG |
| 10178 | TENM1 | HSX2_01477675 | 831 | GTGATGGTGGCTATGCCAAAGATGCAAAGATGAAAGCCCCTTCCTCCTTAGC |
| | | | | AGTGTCGCCTGAT |
| | | | | GGAACCCTCTATGTGGCAGACCTCGGAAATGTTCGAATTCGTACCATCAGCA |
| | | | | GGAACCAAGCCCA |
| | | | | CCTGAATGACATGAACATTTATGAGATTGCTTCACCCGCTGATCAGGAACTG |
| | | | | TACCAGTTCACTGT |
| | | | | AAATGGAACCCACCTACACACCCTGAACTTGATAACAAGGGACTATGTTTAT |
| | | | | AACTTCACCTACAA |
| | | | | TTCTGAAGGTGACTTGGGCGCGATTACCAGCAGCAATGGCAATTCAGTGCA |
| | | | | CATTCGCCGTGATG |
| | | | | CAGGCGGAATGCCGCTATGGCTTGTGGTGCCTGGCGGACAAGTATACTGGC |
| | | | | TGACTATAAGCAG |
| | | | | CAATGGAGTCCTGAAAAGAGTGTCAGCCCAAGGCTATAATCTGGCCTTAAT |
| | | | | GACCTATCCAGGAA |
| | | | | ACACAGGGCTTCTGGCTACCAAAAGTAACGAAAATGGATGGACAACCGTTT |
| | | | | ATGA |
| 10178 | TENM1 | HSX2_01477685 | 832 | AAAATACTCAAAGTACCTATCGGGTGAATCCAGATGGTTCCCTGCGTGTCAC |
| | | | | TTTTGCCAGCGGG |
| | | | | ATGGAGATCGGCCTCAGCTCAGAGCCCCACATCCTGGCAGGGGCAGTCAAC |
| | | | | CCTACCCTGGGCA |
| | | | | AATGCAACATCTCATTGCCCGGAGAGCACAATGCAAACCTCATCGAGTGGC |
| | | | | GGCAGAGGAAGGA |
| | | | | GCAAAACAAAGGCAATGTTTCGGCTTTTGAAAGGAGGCTGAGG |
| 10178 | TENM1 | HSX2_01477690 | 833 | GCCCACAACAGAAACCTACTCTCCATAGATTTTGATCATATAACCCGCACAG |
| | | | | GAAAGATCTATGAT |
| | | | | GACCATCGAAAATTCACCCTTCGAATTCTTTATGACCAGACTGGGCGACCCA |
| | | | | TTCTGTGGTCTCCT |
| | | | | GTAAGCAGATATAATGAAGTGAACATCACATATTCACCTTCGGGATTGGTGA |
| | | | | CGTTTATTCAAAG |
| | | | | AGGAACGTGGAATGAAAAAATGGAATATGACCAGAGTGGGAAAATTATTTC |
| | | | | AAGAACTTGGGCT GATGGGAAAATTTGGAGCTATACCTACTTAGAAAAA |
| 10178 | TENM1 | HSX2_01477695 | 834 | TCTGTGATGCTTCTCCTACACAGCCAGCGGCGTTACATCTTTGAGTATGACCA |
| | | | | ACCAGATTGTCTG |
| | | | | CTCTCAGTCACCATGCCTAGCATGGTGCGTCACAGCTTACAAACCATGCTTTC |
| | | | | AGTGGGCTACTAC |
| | | | | CGGAACATCTACACCCCACCGGACAGTAGCACTTCTTTTATCCAAGACTATA |
| | | | | GTCGAGATGGCCG |
| | | | | ATTGCTACAGACCCTGCATCTGGGGACAGGGCGCAGAGTCTTATACAAGTA |
| | | | | CACCAAGCAAGCA |
| | | | | AGGCTTTCTGAGGTTCTCTATGATACCACTCAGGTCACATTAACATATGAAG |
| | | | | AGTCTTCTGGAGTG |
| | | | | ATTAAGACAATACACCTGATGCATGACGGATTCATCTGCACAATCAGATACA |
| | | | | GGCAAACAG |
| 10178 | TENM1 | HSX2_01477700 | 835 | GACCTCTTATTGGACGCCAGATTTTCAGATTCAGTGAAGAAGGCCTTGTGAA |
| | | | | TGCACGGTTCGAC |
| | | | | TACAGCTACAACAATTTCCGAGTCACAAGCATGCAAGCTGTAATCAATGAAA |
| | | | | CCCCTTTGCCTATA |
| | | | | GATCTTTACCGATATGTTGATGTCTCTGGCAGAACAGAGCAGTTTGGAAAAT |
| | | | | TCAGTGTAATTAAT |
| | | | | TACGATTTAAATCAGGTCATAACTACTACAGTGATGAAACACACCAAAATCT |
| | | | | TCAGTGCCAATGGA |
| | | | | CAAGTCATTGAAGTCCAATATGAAATCCTAAAGGCAATTGCCTACTGGATGA |
| | | | | CCATTCAATATGAT |
| | | | | AATGTGGGCCGCATGGTAATATGTGACATAAGGGTAGGAGTAGATGCCAAT |
| | | | | ATAACAAGGTACT |
| | | | | TCTATGAATACGATGCTGATGGGCAACTTCAGACTGTTTCTGTAAATGACAA |
| | | | | AACCCAGTGGCGT |
| | | | | TATAGTTACGATCTGAATGGAAACATCAACCTCTTAAGCCATGGGAAGAGTG |
| | | | | CTCGTCTTACTCCT |
| | | | | CTCCGATATGACCTCCGAGACCGCATCACCAGATTAGGAGAAATTCAGTATA |
| | | | | AAATGGATGAAGA |
| | | | | TGGCTTTCTGAGGCAGAGGGGAAATGATATTTTTGAATATAATTCTAATGGC |
| | | | | CTGCTGCAGAAAG |
| | | | | CCTACAATAAGGCTTCTGGCTGGACTGTGCAGTATTACTATGATGGGCTTGG |
| | | | | GCGACGTGTCGCG |
| | | | | AGTAAGTCCAGCCTAGGGCAGCACCTTCAGTTCTTTTATGCAGACCTTACCA |
| | | | | ACCCCATAAGAGTT |
| | | | | ACTCATTTGTACAACCACACAAGCTCGGAGATTACATCTCTGTATTATGATCT |
| | | | | CCAAGGTCACCTT |
| | | | | ATTGCCATGGAGTTAAGCAGTGGTGAAGAATATTATGTAGCCTGTGATAATA |
| | | | | CAGGTACCCCACT |
| | | | | AGCTGTGTTCAGCAGCCGAGGTCAGGTCATAAAGGAGATACTATACACACC |
| | | | | TTATGGCGATATCT |
| | | | | ATCATGACACTTACCCTGACTTTCAGGTCATTATTGGTTTTCATGGAGGACTC |
| | | | | TATGATTTCCTTAC |
| | | | | TAAATTAGTGCACCTGGGGCAAAGGGATTATGATGTTGTTGCTGGCAGATG |
| | | | | GACAACGCCTAATC |
| | | | | ATCACATATGGAAACAGTTGAACCTCCTTCCTAAACCATTCAACCTCTACTCC |
| | | | | TTTGAAAATAACTA |
| | | | | CCCAGTTGGCAAAATTCAAGATGTTGCAAAGTATACCACAG |
| 10178 | TENM1 | HSX2_01477705 | 836 | ACATCAGAAGTTGGTTGGAGCTATTTGGTTTCCAATTACACAATGTACTACCT |
| | | | | GGATTTCCCAAAC |
| | | | | CTGAATTAGAAAATTTAGAATTAACTTACGAGCTTCTACGGCTTCAGACAAA |
| | | | | AACTCAAGAGTGGGATCCTGGAAAG |
| 10178 | TENM1 | HSX2_01477710 | 837 | ACTATCCTGGGCATTCAGTGTGAACTCCAGAAACAGCTCAGGAATTTCATTT |
| | | | | CCTTGGACCAACTA |
| | | | | CCTATGACTCCCCGATACAATGATGGACGGTGCCTTGAAGGAGGGAAGCAA |
| | | | | CCAAGGTTTGCTG |
| | | | | CTGTCCCTTCTGTTTTTGGGAAAGGTATAAAATTTGCCATCAAGGATGGCAT |
| | | | | AGTAACAGCTGATA |
| | | | | TTATAGGAGTAGCCAATGAAGATAGCAGGCGGCTTGCTGCCATTCTCAATAA |
| | | | | TGCCCATTACCTG |
| | | | | GAAAACCTACATTTTACCATAGAGGGGAGGGACACTCACTACTTCATTAAGC |
| | | | | TTGGGTCTCTGGA |
| | | | | GGAAGACCTGGTGCTCATCGGTAACACTGGGGGGAGGCGGATTCTGGAGA |
| | | | | ATGGTGTCAATGTC |
| | | | | ACTGTGTCCCAGATGACTTCTGTGTTGAATGGGAGGACTAGACGGTTTGCA |
| | | | | GATATTCAGCTCCA |
| | | | | GCATGGAGCCCTGTGCTTCAACATCCGGTATGGGACAACTGTCGAAGAGGA |
| | | | | AAAGAATCACGTG |
| | | | | TTGGAGATTGCCAGACAGCGCGCAGTGGCCCAGGCCTGGACTAAGGAACAA |
| | | | | AGAAGGCTGCAA |
| | | | | GAGGGGGAAGAGGGGATTAGGGCATGGACAGAAGGGGAAAAGCAGCAGC |
| | | | | TTTTGAGCACTGG |
| | | | | GCGGGTACAAGGTTACGATGGGTATTTTGTTTTGTCTGTTGAGCAGTATTTA |
| | | | | GAACTTTCTGACAG |
| | | | | TGCCAATAATATTCACTTTATGAGACAGAGCGAAATAGGCAGGAGGTAACA |
| | | | | AAAATATCTCTGCC TTTGCGTCACCAA |
| 10178 | TENM1 | HSX2_01477711 | 838 | GTAAATATGGAGGAAAAACATATCCAACTGCCTTTCAATGTGACGGAAGAT |
| | | | | GGTATTTTAATATT |
| | | | | GTTTGTTTAAACTCTTTAAGAAATGACAGAGATTTTTAGTTCTTGTGTGGCAG |
| | | | | TATTCAAAATAAC |
| | | | | ACAAGTAGAACTCAAACAGCTAAAAACAGTTTTCAGAAAGCACCACTTTCAA |
| | | | | TTTGCCGAGCCAT |
| | | | | GCATATGTTCCAATATCCAGAAAGAACCCAAGGTTCTCTATCTCTATTGTGAG |
| | | | | AAGCAGTTTCATC |
| | | | | CTTAACTGTTGGCAGAACTTACGGGCTATTTGAATAGGTGGTGCAATAGTAT |
| | | | | CTGAAACTTGCCTT |
| | | | | TCGAAAGACTGCCAGCCCTTTGACGTTTTCCAGATCTGTTATAGGAAACTTA |
| | | | | AAAACAGGTGTAA |
| | | | | AATGTCTTCAGCCACCATCTCCTAGAGTGAGGACCCAATTGCCCTTCCTTCTT |
| | | | | GATTATTCCTCCTT |
| | | | | GCTTGTTAAAGTAAATGCCATATTGTTGTGCTGTGTTTTGGCGTGTGGTGGC |
| | | | | TGGGTTCTGTCTAC |
| | | | | CATGCTTCCCTGTGGGTGTGGTAACCAGACTGTATAGCCGCTATTTGCTCGT |
| | | | | GTGTACATGATACC |
| | | | | AAAGCAGCTGGCCAGCGTGACCTCTCTCACACGACCTGTTTTGACTCAATTTT |
| | | | | TTACTAAAAGTTG |
| | | | | TTCAGCTGTATTGGTATCATGTAAACATAGCTTTTATTAACCTGGGTAGGAAT |
| | | | | TTCTCATTTATATA |
| | | | | TAGGATGTGTTTTGGTCATAGTTTCACATTAGTGATTCAGTATCTATACACTG |
| | | | | ACCCAATGGTTTTG |
| | | | | TGCACATGAACGGTAATTTACTTAAAAGTATGATTCTGGTACAAAAACAAAC |
| | | | | AAAGGCTTTAGCA |
| | | | | GGCATACGTGTCTGGGATGCCGATACATACATTAACTACTACTGCAGAAATT |
| | | | | CATAAGAGCCAAA |
| | | | | ACCTTAAAAAAATAGACCTGGTACTTAAGTGAAAGTACTAAAGGGAAGACC |
| | | | | AGACCAAACATCAC |
| | | | | AGCAGTTGCTGCCACATTGTTTCAGCCCACTTAGATTTATCTTTCAAATGTAC |
| | | | | AATTCTGTATTGAA |
| | | | | CATCTCCCAGCCATCTTCAGGAAATCGAATCAAGTAAATCCTTTCCAACCGAA |
| | | | | AACATTTCAACTA |
| | | | | ACTATAGAGAGGCAGACTCATTTTTACTAAAATAATTTATACAGTTAGTTATT |
| | | | | TTCGTTCTCCGTAC |
| | | | | TTACCCATTTATCTTTATTTAATCGTCTCTACTGCCTAGGAAAATAACTATTTT |
| | | | | CCAGGACGGGTTA |
| | | | | TTTGTTCTGCGATCATTTAAAATTTGGAGAAAGGTCAGGATTAGTGTTAATA |
| | | | | TCAGCTGCAGTTTC |
| | | | | TCAATCTCTAGGAATCCTGCAGTAAAACAAGCCCCTTGGTGAGCTGGAAGAT |
| | | | | TTGTGCCCAGTGA |
| | | | | CAAAGAGATAGTTTGTAAAATGCTGTGTAATTGTAAGTTACCACAAATGAAA |
| | | | | ATACATGACAGCA |
| | | | | CAATGTGGCCCGTAGAAAATTCCCCTGAGCCAGCTTCTGCACTTTCATCACC |
| | | | | GAATCTGAACATTT |
| | | | | GCTATGTCTGAAGGCAAATTTATGATGGAATGTTAGTTTGGATTCTTTCCAG |
| | | | | ATGCTACCTAAATG |
| | | | | CAGTGTGGGGTCATTGCCTTGCTTTGCGATGACAGTTTCTTTGAAAATATGC |
| | | | | AAAGTCATAAGCTC |
| | | | | ATGTTAAGGTTTTTCAAGAGTCTGCCTCCTACTACACAAAGGAAAGCAAGGG |
| | | | | AAAGGAAATGACC |
| | | | | CTGGCAAACAGTAGGGAAGGGTGTATTCAAACATTTCATTTTCAAAACCTTC |
| | | | | GGGTTAGAATACC |
| | | | | ACTTACACATGTATTCTGAGAGACAGAATTCATGAGGAACTCATCTCTCTTTA |
| | | | | TAACTGGAAACAC |
| | | | | ACCAGCTTGATATATTGCTAATCCATACTAAAATCATATTATTGGGTTTTTTCT |
| | | | | GAATCAGGCCTGT |
| | | | | ATTAATGGTACAGTATTTATTCAGAATGGAATTCTAAAATTACTAACAAACTT |
| | | | | GTTGAAAATTTGA |
| | | | | ATACCTCCACACCAACCTAAAAATGGACCTTAAGTTCCTAGAACCTCTGATGT |
| | | | | TCTTTTAAATTAAT |
| | | | | GGAAAAATAATTTGTGAACTGTATATAGAGAGTGCATTCATAAATGTGATTA |
| | | | | TGTATTTTATCACA |
| | | | | AATCCAAAATGTCAATATTAGAGTCTATTTTGCTTATATTTTAAGCAATTATA |
| | | | | CGTTTTTGCAATTCA |
| | | | | TTGATGATGTATCATTTTCAAACTGCTTTAAATATCCATTAGAAACAAATATT |
| | | | | TGAAGCTTTTACTT |
| 29842 | TFCP2L1 | HSX2_01723004 | 839 | CCGAAGGGAGCCGATCGGAAACAGAAGACTGACCGGGAGAAGATGGAGA |
| | | | | AAAGAACTGCCCAA |
| | | | | GAGAAGGAGAAATACCAGCCGTCCTATGAAACCACCATCCTCACAGAG |
| 7039 | TGFA | HSX2_01263636 | 840 | TGGTCTGAAGAGCCCAGAGGAGGAGTTTGGCCAGGTGGACTGTGGCAGAT |
| | | | | CAATAAAGAAAGG |
| | | | | CTTCTTCAGGACAGCACTGCCAGAGATGCCTGGGTGTGCCACAGACCTTCCT |
| | | | | ACTTGGCCTGTAA |
| | | | | TCACCTGTGCAGCCTTTTGTGGGCCTTCAAAACTCTGTCAAGAACTCCGTCTG |
| | | | | CTTGGGGTTATTC |
| | | | | AGTGTGACCTAGAGAAGAAATCAGCGGACCACGATTTCAAGACTTGTTAAA |
| | | | | AAAGAACTGCAAA |
| | | | | GAGACGGACTCCTGTTCACCTAGGTGAGGTGTGTGCAGCAGTTGGTGTCTG |
| | | | | AGTCCACATGTGT |
| | | | | GCAGTTGTCTTCTGCCAGCCATGGATTCCAGGCTATATATTTCTTTTTAATGG |
| | | | | GCCACCTCCCCACA |
| | | | | ACAGAATTCTGCCCAACACAGGAGATTTCTATAGTTATTGTTTTCTGTCATTT |
| | | | | GCCTACTGGGGAA |
| | | | | GAAAGTGAAGGAGGGGAAACTGTTTAATATCACATGAAGACCCTAGCTTTA |
| | | | | AGAGAAGCTGTAT |
| | | | | CCTCTAACCACGAGACCCTCAACCAGCCCAACATCTTCCATGGACACATGAC |
| | | | | ATTGAAGACCATCC |
| | | | | CAAGCTATCGCCACCCTTGGAGATGATGTCTTATTTATTAGATGGATAATGG |
| | | | | TTTTATTTTTAATCT |
| | | | | CTTAAGTCAATGTAAAAAGTATAAAACCCCTTCAGACTTCTA |
| 7048 | TGFBR2 | HSX2_01264514 | 841 | GAGAAAGAATGACGAGAACATAACACTAGAGACAGTTTGCCATGACCCCAA |
| | | | | GCTCCCCTACCATG |
| | | | | ACTTTATTCTGGAAGATGCTGCTTCTCCAAAGTGCATTATGAAGGAAAAAAA |
| | | | | AAAGCCTGGTGAG |
| | | | | ACTTTCTTCATGTGTTCCTGTAGCTCTGATGAGTGCAATGACAACATCATCTT |
| | | | | CTCAGAAG |
| 7069 | THRSP | HSX2_01266503 | 842 | ATCCCTTCACATGATAGAAGACAGACTCTTTGATGAGGTCGGCGGAGCAGTT |
| | | | | CACTAGCCAATGA |
| | | | | TGAGAGCAGAAAGGCCTAGACCTGCAGCCAGAAGTGAAGGCGGCTCAGTT |
| | | | | CTCCGGGATGCTTC |
| | | | | TCTACCTCCTGAGCACCAATTCCTGGATTCCAGTCACTGGCTCACCTTTAGAA |
| | | | | TGTCTGTTGCTATT |
| | | | | CACTGCTCCCCTCGCTCCTCTTAACAGCTTGGGGAGGTGACCAGTGGTTCAG |
| | | | | GAGGGACTAGACA |
| | | | | ATTACCTGTCCAGTGTGGTATGGTAGGAAGAGTGTAGGTGTTGGCACGTGA |
| | | | | CCAAAATTCACATC |
| | | | | CCTCCTCATGGCAGTCATTCAGTATGTGTACTTGTACAAGTTATTTAACCCAT |
| | | | | TGGAGCCTAAATTC |
| | | | | CCTCATCTATAAAATGGGGATAATATTATCTACCTCACAAGCTTATGAAAACT |
| | | | | AAACATGATGAAT |
| | | | | CAAAAGCACTTGGCATGTGAGGGCTATTAAAATAGCCTGATTTTTTTTTTCTC |
| | | | | CCCCTCTCCCCAAT |
| | | | | GTATTTGCTCTGGCCCTTGCTTTTTACCCTCCAGAGCTAAGAGGTAGCAGAG |
| | | | | TCTCTTGGGATGAG |
| | | | | TGATTCACCCTCTTACTTGGCGACCACTGATGAGATCAACAACAGGTGAACT |
| | | | | ATAAACCTATTATT TATTGCAGAACTAATAAAAAATCCAAAGCCTTGTA |
| 25976 | TIPARP | HSX2_01671479 | 843 | GATTTTTAGACTCTGAGGAGCAGTTGGAGCTAATCCACATTATGGAAATGGAA |
| | | | | ACCACCGAACCT |
| | | | | GAGCCAGACTGTGTAGTGCAGCCTCCCTCTCCTCCTGATGACTTTTCATGCCAA |
| | | | | ATGAGACTCTCT |
| | | | | GAGAAGATCACTCCATTGAAGACTTGTTTTAAGAAAAAGGATCAGAAAAGATT |
| | | | | GGGAACTGGAA |
| | | | | CCCTGAGGTCTTTGAGGCCAATATTAAACACTCTTCTAGAATCTGGCTCACTTG |
| | | | | ATGGGGTTTTTA |
| | | | | GATCTAGGAACCAGAGTACAGATGAGAACAGCTTACATGAACCTATGATGAAG |
| | | | | AAAGCCATGGA |
| | | | | AATCAATTCATCATGCCCACCAGCAGAAAATAATATGTCTGTTCTGATTCCTGAT |
| | | | | AGGACAAATGT |
| | | | | TGGGGACCAGATACCGGAAGCCCATCCTTCCACTGAAGCTCCAGAACGAGTGG |
| | | | | TTCCAATCCAA |
| | | | | GATCACAGCTTTCCATCAGAAACCCTCAGTGGGACGGTGGCAGATTCCACACC |
| | | | | AGCTCACTTCCA |
| | | | | GACTGATCTTTTGCACCCAGTTTCAAGTGATGTTCCTACTAGTCCTGACTGCTTA |
| | | | | GACAAAGTCAT |
| | | | | AGATTATGTTCCAGGCATTTTCCAAGAAAACAGTTTTACAATCCAATACATTCTG |
| | | | | GACACCAGTGA |
| | | | | TAAGCTGAGTACTGAGCTCTTTCAGGACAAAAGTGAAGAGGCTTCCCTTGACCT |
| | | | | CGTGTTTGAGC |
| | | | | TGGTGAACCAGTTGCAGTACCACACTCACCAAGAGAACGGAATTGAAATTTGC |
| | | | | ATGGACTTTCTG |
| | | | | CAAGGCACTTGTATTTATGGCAGGGATTGTTTGAAGCACCACACTGTCTTGCCA |
| | | | | TATCATTGGCAG |
| | | | | ATCAAAAGGACAACTACTCAAAAGTGGCAGAGTGTATTCAATGATTCTCAGGA |
| | | | | GCACTTGGAAAG |
| | | | | ATTTTACTGTAACCCAGAAAATGATAGAATGAGAATGAAGTATGG |
| 25976 | TIPARP | HSX2_01671483 | 844 | AGGACAAGAATTTTGGGCAGATTTGAATGCCATGAACGTGTATGAAACAACTG |
| | | | | AATTTGACCAAC |
| | | | | TACGAAGGCTGTCCACACCACCCTCTAGCAATGTCAACTCTATTTACCACACAGT |
| | | | | CTGGAAATTCT TCTGTAGGGACCACTTTGGATGGAGAGAGTATCCCGAG |
| 25976 | TIPARP | HSX2_01671512 | 845 | GAAAAAGGAATATATGAACAGGAAAATGTTTGGCCGTGACAGGATAATAAAT |
| | | | | GAGAGACATTTA |
| | | | | TTTCATGGAACATCCCAGGATGTGGTAGATGGAATCTGCAAACACAACTTTGAC |
| | | | | CCTCGAGTCTG |
| | | | | TGGAAAGCATGCTACAATGTTTGGACAAGGCAGTTATTTTGCAAAGAAGGCAA |
| | | | | GCTACTCTCATA |
| | | | | ACTTTTCTAAGAAGTCCTCCAAAGGAGTCCACTTCATGTTTCTGGCCAAAGTGC |
| | | | | TGACGGGCAGA |
| | | | | TACACAATGGGCAGTCATGGCATGAGAAGGCCCCCGCCAGTCAATCCTGGCAG |
| | | | | TGTCACCAGTG |
| | | | | ACCTTTATGACTCTTGTGTGGATAATTTCTTTGAGCCTCAGATTTTTGTCATTTTT |
| | | | | AATGATGACCA |
| | | | | GAGTTACCCTTATTTTGTTATCCAATATGAAGAAGTCAGTAACACTG |
| 9414 | TJP2 | HSX2_01405349 | 846 | AAAACGCACAAGCCAGACCCTGGCACGCCCCAGCACACGAG |
| 7088 | TLE1 | HSX2_01268117 | 847 | GACCCTTCTTCTCCGCGAGCAAGCCCTGCCCACTCGCCCCGGGAAAATGGAATC |
| | | | | GACAAAAATCG |
| | | | | CCTGCTAAAGAAGGATGCTTCTAGCAGTCCAGCTTCCACGGCCTCCTCG |
| 7088 | TLE1 | HSX2_01268172 | 848 | TCCAAAGAGTCCTCGTCAGTGCTTAGCTGTGACATCTCTGTGGATGATAAGTAC |
| | | | | ATAGTCACTGG |
| | | | | CTCGGGGGACAAGAAGGCTACAGTCTATGAAGTCATCTACTGAAAACATTATG |
| | | | | TGGTTTAACGTT |
| | | | | TATAGTTGAATTGGGCCAAAATGTTTCGAATTTATAGAAATAGAAAAGTTGTAA |
| | | | | CTTTAAAAGAG |
| | | | | AAAAAAAATTACAAACACCTGTTTCCAAACCTTGACAGAAAACTACTTTGAGTC |
| | | | | TACAAAGAGGA |
| | | | | GGCGACAAGTCCATCAGCAGAAAGTCACCTGTCTACATAGACCAAATGGAGCA |
| | | | | CCAAGGCCAAG |
| | | | | CGGACAGAGGGGCCATGGGTTGTAGGATTGAGGAACGGAATCTGCCGACTCA |
| | | | | CATGACAGCCC |
| | | | | ATTCTTTCTTTCTGGGTGATCTGGGGATCACGCCTTGCCCAAGTGTGAGATTAC |
| | | | | CTTTCTGTTCCTT |
| | | | | GCAGTTCACCTCACTTTCCGTCCTTTGTAGAGCAGTGGTGTCTCCAATGAACTTG |
| | | | | TTTCCTGGTTTT |
| | | | | GCATCTTGTGAAATGTTTTTTTGTATTTTTGTTGAAGGTTAAACATTTGTATAAA |
| | | | | TTGTAAATATATT TGGTTTATTACAGTAAAGGCTTTAGTACCAATAA |
| 83660 | TLN2 | HSX2_02072578 | 849 | CCTGGAAGCAAGATGGTGTCCTCTGCCAAAGCCGCAGTGCCCACCGTGAGTGA |
| | | | | CCAGGCCGCAG |
| | | | | CCATGCAGCTGAGCCAGTGTGCCAAGAACCTGGCCACCAGCTTGGCGGAGCTG |
| | | | | CGTACCGCCTC GCAGAAG |
| 25963 | TMEM87A | HSX2_01670934 | 850 | TGATGGAGAACCTTGTGACCTGTCTTTGAATATAACCTGGTATCTGAAAAGCGC |
| | | | | TGATTGTTACAA TGAAATCTATAACTTCAAG |
| 25963 | TMEM87A | HSX2_01670939 | 851 | CACTATCTTCCTGAAGTTTGATGGAGAACCTTGT |
| 25963 | TMEM87A | HSX2_01670944 | 852 | GAAGTAGAGTTGTATTTGGAAAAACTTAAGGAAAAAAGAGGCTTGTCTGGGA |
| | | | | AATATCAAACATC |
| | | | | ATCAAAATTGTTCCAGAACTGCAGTGAACTCTTTAAAACACAG |
| 25963 | TMEM87A | HSX2_01670955 | 853 | ACCTTTTCTGGAGATTTTATGCATCGACTGCCTCTTTTAGGAGAAAAAC |
| 25963 | TMEM87A | HSX2_01671067 | 854 | TTGGATTGGTGCTGTCATCTTCCTGGGAATGCTT |
| 25963 | TMEM87A | HSX2_01671111 | 855 | GGATATGGCATCGTCAAGCCACGCCTTGGAGTCA |
| 25963 | TMEM87A | HSX2_01671149 | 856 | GCCTTGTGCTGGTGGATATTTATTAGCCTGACTC |
| 25963 | TMEM87A | HSX2_01671180 | 857 | TAGTGACATGTCAGTCGGACTGGCGGGAGCTGTG |
| 25963 | TMEM87A | HSX2_01671193 | 858 | GCCTTTTCACCATTGTCTGAGGAAGAGGAGGAGGATGAACAAAAGGAGCCTAT |
| | | | | GCTGAAAGAAA GGCTTTG |
| 25963 | TMEM87A | HSX2_01671207 | 859 | TCTGCAAACAACCAGAGGTTTGCCTTTTCACCAT |
| 25963 | TMEM87A | HSX2_01671265 | 860 | GAACGAATGATCACACACTTTGAAAGGTCCAAAATGGAGTAAGGAATGGGAA |
| | | | | GATTTGCAGTTA |
| | | | | AAGATGGCTACCATCAGGGAAGAGATCAGCATCTGTGTCAGTCTTCTGTACGG |
| | | | | CTCCATGGGATT |
| | | | | AAAGGAAGCAATGACATCCTGATCTGTTCCTTGATCTTTGGGCATTGGAGTTGG |
| | | | | CGAGAGGTGTC |
| | | | | AGAACAAAGAGAACATCTTACTGAAAACAAGTTCATAAGATGAGAAAAATCTA |
| | | | | CGAGCTTCTTAT |
| | | | | TTACAACACTGCTGCCCCCTTTCCTCCCAGACTCTGACATGGATGTTCATGCAAC |
| | | | | TTAAGTGTGTT |
| | | | | GTTCCTGAACTTTCTGTAATGTTTCATTTTTTAAATCTGACAAACTAAAAAGTTT |
| | | | | AACGTCTTCTAA |
| | | | | AAGATTGTCATCAACACCATAATATGTAATCTCCAGGAGCAACTGCCTGTAATT |
| | | | | TTTATTTATTTAG |
| | | | | GGAGTTACATAGGTGATGGGGGAAATTGTTAACTACCTTTCATTTTCCTGGGAA |
| | | | | GTCAAGGTTAC |
| | | | | ATCTTGCAGAGGTTGTTTTGAGAAAAAAGGGCCCTTCTGAGTTAAGGAGCCAT |
| | | | | AGTTCTATCAAT GATCAA |
| 25963 | TMEM87A | HSX2_02546143 | 861 | TTGATGGAGAACCTTGTGACCTGTCTTTGAATATAACCTGGTATCTGAAAAGC |
| | | | | GCTGATTGTTACA ATGAAATCTATAACTTCAAG |
| 25963 | TMEM87A | HSX2_02546146 | 862 | GCAGAAGAAGTAGAGTTGTATTTGGAAAAACTTAAGGAAAAAAGAGGCTTGT |
| | | | | CTGGGAAATATC |
| | | | | AAACATCATCAAAATTGTTCCAGAACTGCAGTGAACTCTTTAAAACACAG |
| 25963 | TMEM87A | HSX2_02546153 | 863 | ACCTTTTCTGGAGATTTTATGCATCGACTGCCTCTTTTAGGAGAAAAACAGGAG |
| 25963 | TMEM87A | HSX2_02546163 | 864 | TGACTGTTGAAGTGAAGGGTCCCTATGAATACCTCACACTTGAAGACTATCCCT |
| | | | | TGATGATT |
| 26022 | TMEM98 | HSX2_01675662 | 865 | GCCCATTGTGGACCTCATTGGTGCCATGGAGACCCAGTCTGAGCCCTCTGAGT |
| | | | | TAGAACTGGACG |
| | | | | ATGTCGTTATCACCAACCCCCACATTGAGGCCATTCTGGAGAATGAAGACTGG |
| | | | | ATCGAAGAT |
| 26022 | TMEM98 | HSX2_01675678 | 866 | GGGTCTCATGTCCCACTGCATTGCCATCTTGAAG |
| 26022 | TMEM98 | HSX2_02546441 | 867 | ATTTGTCACACTCTGACAGAGAAGCTTGTTGCCATGACAATGGGCTCTGGGGC |
| | | | | CAAGATGAAGAC |
| | | | | TTCAGCCAGTGTCAGCGACATCATTGTGGTGGCCAAGCGGATCAGCCCCAG |
| 51330 | TNFRSF12A | HSX2_01760100 | 868 | GCGCTGCAGCACCTCCTGCCCCCTTCCGGCTGCTTTGGCCCATCCTTGGGGGC |
| | | | | GCTCTGAGCCTG |
| | | | | ACCTTCGTGCTGGGGCTGCTTTCTGGCTTTTTGGTCTGGAGACGATGCCGCAG |
| | | | | GAGAGAGAAGTT CACCA |
| 23043 | TNIK | HSX2_01589734 | 869 | GGGCTGAGTCACCTGCACCAGCATAAAGTGATTCATCGAGATATTAAAGGGCA |
| | | | | AAATGTCTTGCT GACTGAAAATGCAGAAGTTAAACTAG |
| 23043 | TNIK | HSX2_01589742 | 870 | TGGACTTTGGAGTCAGTGCTCAGCTTGATCGAACAGTGGGCAGGAGGAATAC |
| | | | | TTTCATTGGAACT |
| | | | | CCCTACTGGATGGCACCAGAAGTTATTGCCTGTGATGAAAACCCAGATGCCAC |
| | | | | ATATGATTTCAA G |
| 23043 | TNIK | HSX2_01589753 | 871 | CCACATATGATTTCAAGAGTGACTTGTGGTCTTT |
| 23043 | TNIK | HSX2_01589756 | 872 | CTCTCTGTGACATGCACCCCATGAGAGCTCTCTTCCTCATCCCCCGGAACCCAG |
| | | | | CGCCTCGGCTGA AGTCTAAGAAGTG |
| 23043 | TNIK | HSX2_01589779 | 873 | CTCCATCCTGAATCTGCCAGGGGAGTCGACGCTGCGGAGGGACTTTCTGAGGC |
| | | | | TGCAGCTGGCC |
| | | | | AACAAGGAGCGTTCTGAGGCCCTACGGAGGCAGCAGCTGGAGCAGCAGCAG |
| | | | | CGGGAGAATGAG |
| | | | | GAGCACAAGCGGCAGCTGCTGGCCGAGCGTCAGAAGCGCATCGAGGAGCAG |
| | | | | AAAGAGCAGAGGCGGCGGCTGGAGGAG |
| 23043 | TNIK | HSX2_01589799 | 874 | GAATATAAGCGCAAACAATTGGAAGAACAGAGACAAGCAGAAAGACTGCAG |
| | | | | AGGCAGCTAAAG |
| | | | | CAAGAAAGAGACTACTTAGTTTCCCTTCAGCATCAGCGGCAGGAGCAGAGGC |
| | | | | CTGTGGAGAAGA |
| | | | | AGCCACTGTACCATTACAAAGAAGGAATGAGTCCTAGTGAGAAGCCAGCATG |
| | | | | GGCCAAGGAG |
| 23043 | TNIK | HSX2_01589809 | 875 | GTAGAAGAACGGTCAAGGCTCAACCGGCAAAGTTCCCCTGCCATGCCTCACAA |
| | | | | GGTTGCCAACA |
| | | | | GGATATCTGACCCCAACCTGCCCCCAAGGTCGGAGTCCTTCAGCATTAGTGGA |
| | | | | GTTCAGCCTGCT CGAACACCCCCCATGCTCAGACCAGTCGATCCCCAG |
| 23043 | TNIK | HSX2_01589851 | 876 | CCAACAGTAAGTCAGAAGGATCACCTGTGCTCCCCCATGAGCCTGCGAAGGTG |
| | | | | AAACCAGAAGA ATCCAGGGACATTACCCGGCCCAGTCGACCAGCT |
| 23043 | TNIK | HSX2_01589906 | 877 | ACGTCTGGAGAGAAGAAGCGATCTGGCCACAGTGACAGCAATGGCTTTGCTG |
| | | | | GCCACATCAACC |
| | | | | TCCCTGACCTGGTGCAGCAGAGCCATTCTCCAGCTGGAACCCCGACTGAGGGA |
| | | | | CTGGGGCGCGT CTCAACCCATTCCCAGGAGATGGACTCTGGGACTGAA |
| 23043 | TNIK | HSX2_01589937 | 878 | CTCTGTTTACTAGCGAACTTCTTAGGCAAGAACAGGCCAAACTCAATGAAGCA |
| | | | | AGAAAGATTTCG |
| | | | | GTGGTAAATGTAAACCCAACCAACATTCGGCCTCATAGCGACACACCAGAAAT |
| | | | | CAGAAAATACAA GAAACGATTCAACTCAGAAATACTTTGTGCAGCTCTGTGGG |
| 23043 | TNIK | HSX2_01590045 | 879 | CCTACATTCATTCCAATCAGATAATGGGCTGGGGCGAGAAAGCTATTGAGATC |
| | | | | CGGTCAGTGGAA |
| | | | | ACAGGACATTTGGATGGAGTATTTATGCATAAGCGAGCTCAAAGGTTAAAGTT |
| | | | | TCTATGTGAAAG AAATGATAAG |
| 7159 | TP53BP2 | HSX2_01274309 | 880 | GCCTCGGATGCCCTCAAGGCCTGAATTGCTGGTGAAGCCAGCCCTGCCGGATG |
| | | | | GTTCCTTGGTCA |
| | | | | TTCAGGCTTCAGAGGGGCCGATGAAAATACAGACACTGCCCAACATGAGATCT |
| | | | | GGGGCTGCTTC ACAAACTAAAG |
| 7159 | TP53BP2 | HSX2_01274325 | 881 | GTTGCAAATAAAAATGTGGCTAAAGTACCACCTCCTGTTCCTACAAAACCAAAA |
| | | | | CAGATTAATTTG |
| | | | | CCTTATTTTGGACAAACTAATCAGCCACCTTCAGACATTAAGCCAGACGGAAGT |
| | | | | TCTCAGCAGTTG |
| | | | | TCAACAGTTGTTCCGTCCATGGGAACTAAACCAAAACCAGCAGGGCAGCAGCC |
| | | | | GAGAGTGCTGC |
| | | | | TATCTCCCAGCATACCTTCGGTTGGCCAAGACCAGACCCTTTCTCCAGGTTCTA |
| | | | | AGCAAGAAAGTC |
| | | | | CACCTGCTGCTGCCGTCCGGCCCTTTACTCCCCAGCCTTCCAAAGACACCTTACT |
| | | | | TCCACCCTTCA |
| | | | | GAAAACCCCAGACCGTGGCAGCAAGTTCAATATATTCCATGTATACGCAACAG |
| | | | | CAGGCGCCAGG |
| | | | | AAAAAACTTCCAGCAGGCTGTGCAGAGCGCGTTGACCAAGACTCATACCAGA |
| | | | | GGGCCACACTTTT CAAGTG |
| 7164 | TPD52L1 | HSX2_01274704 | 882 | TACTCCATTCGCCATTCCATAAGTATGCCTGCTATGAG |
| 7164 | TPD52L1 | HSX2_02522322 | 883 | TTACTCCATTCGCCATTCCATAAGTATGCCTGCTATGAG |
| 55521 | TRIM36 | HSX2_01854056 | 884 | GTTACCATTAAGAATATCGAAAGGGAGCTCATTTGCCCAGCATGCAAGGAGCT |
| | | | | GTTTACCCACCC |
| | | | | ATTGATTCTCCCTTGCCAACATAGTATCTGTCATAAATGTGTAAAAGAACTCCT |
| | | | | GCTGACTCTCGAT |
| | | | | GATTCATTCAACGATGTGGGATCAGACAACTCCAATCAAAGCAGTCCTCGACT |
| | | | | TCGGCTCCCCTCC CCTAGTATGGATAAAATTGACCGAATTAACAGACCAG |
| 286144 | TRIQK | HSX2_02489942 | 885 | GCCTTGGCAGAGCTGAGCAGGCATTTTGGAGATC |
| 8848 | TSC22D1 | HSX2_01365731 | 886 | GGTGTTTTTGGCTGCAATTGCATGAAATCCCAATGGTGTAGACCAGTGGCGAT |
| | | | | GGATCTAGGAGT |
| | | | | TTACCAACTGAGACATTTTTCAATTTCTTTCTTGTCATCCTTGCTGGGGACTGAA |
| | | | | AACGCTTCTGTGAGACTTGATAATAG |
| 100131187 | TSTD1 | HSX2_02584821 | 887 | CGCCCACGGTCTCGCTTCCTGAACTCCGTTCACTCCTAGCCTCCGGACGGGCCC |
| | | | | GGCTCTTCGAC |
| | | | | GTGCGCTCTCGCGAGGAGGCGGCAGCTGGGACCATCCCAGGGGCGCTCAACA |
| | | | | TCCCGG |
| 7991 | TUSC3 | HSX2_01317834 | 888 | CGGGTTTTCAGACCACCCAACTACTCTGGTACCATTGCTTTGGCCCTGTTAGTG |
| | | | | TCGCTTGTTGGA |
| | | | | GGTTTGCTTTATTTGAGAAGGAACAACTTGGAGTTCATCTATAACAAGACTGG |
| | | | | TTGGGCCATGGT GTCTCTG |
| 7991 | TUSC3 | HSX2_01317841 | 889 | CTATGACTTCTGGCCAGATGTGGAACCATATCCGTGGACCTCCATATGCTCATA |
| | | | | AGAACCCACAC AATGGACAAGTG |
| 7991 | TUSC3 | HSX2_01317865 | 890 | TTCGTTCCAAGTACCACGGCTATCCTTATA |
| 7991 | TUSC3 | HSX2_01317884 | 891 | TGATCTGGACTTTGAGTGAGAAGATGTGATTTGGACCATGGCACTTAAAAACT |
| | | | | CTATAACCTCAG |
| 7991 | TUSC3 | HSX2_02525011 | 892 | ATTCGGGTTTTCAGACCACCCAACTACTCTGGTACCATTGCTTTGGCCCTGTTA |
| | | | | GTGTCGCTTGTT |
| | | | | GGAGGTTTGCTTTATTTGAGAAGGAACAACTTGGAGTTCATCTATAACAAGAC |
| | | | | TGGTTGGGCCAT GGTGTCTCTG |
| 7991 | TUSC3 | HSX2_02525012 | 893 | TGTATAGTCTTTGCTATGACTTCTGGCCAGATGTGGAACCATATCCGTGGACCT |
| | | | | CCATATGCTCAT AAGAACCCACACAATGGACAAGTG |
| 7991 | TUSC3 | HSX2_02525016 | 894 | CTTTTTAATTAAATGAAGCCAAGTGGGATTTGCATAAAGTGAATGTTTACCATG |
| | | | | AAGATAAACTGT |
| | | | | TCCTGACTTTATACTATTTTGAATTCATTCATTTCATTGTGATCAGCTAGCTTATT |
| | | | | CTTGTGTACTTT |
| | | | | TTTTAAACTGTGGGTTTTCCTAGTAAATTTAATTTACAGAAATCAATGGTAGCA |
| | | | | TTTAGTAATCTAC |
| | | | | AAAGGAAATATCAAAGTGTTTTTCAAGCCTGTTATATTCAGTGTGTGCCACAG |
| | | | | GATTGAAATAAAT GACAATGTAATTATGAA |
| 51377 | UCHL5 | HSX2_01762885 | 895 | GCTCATTAAAGGATTCGGTTGCCGAGGAGCCCAA |
| 51377 | UCHL5 | HSX2_01762887 | 896 | GCCAGTTCATGGGTTAATTTTTCTTTTCAAGTGGCAGCCAGGAG |
| 51377 | UCHL5 | HSX2_01762911 | 897 | GTAATTAATAATGCTTGTGCTACTCAAGCCATAGTGAGTGTGTTACTGAACTGT |
| | | | | ACCCACCAGGAT |
| | | | | GTCCATTTAGGCGAGACATTATCAGAGTTTAAAGAATTTTCACAAAGTTTTGAT |
| | | | | GCAGCT |
| 51377 | UCHL5 | HSX2_01762923 | 898 | ATGAAAGGCTTGGCACTGAGCAATTCAGATGTGATTCGACAAGTACACAACAG |
| | | | | TTTCGCCAG |
| 51377 | UCHL5 | HSX2_01762946 | 899 | GTGCATGCAATCAAGATGATTGGATCAGTGCAGTAAGGCCTGTCATAGAAAAA |
| | | | | AGGATACAAAA |
| 51377 | UCHL5 | HSX2_01762958 | 900 | GTACAGTGAAGGTGAAATTCGATTTAATTTAATGGCCATTGTGTCTGACAGAA |
| | | | | AAATGATATATG AGCAGAAGATAGCAGAGTTACAAAGACAACTTGCAGAG |
| 51377 | UCHL5 | HSX2_01763188 | 901 | GCCGCCGGGGATTCCAGGTTGCCGAGGAGCCCAA |
| 51377 | UCHL5 | HSX2_02551898 | 902 | GCCAGTTCATGGGTTAATTTTTCTTTTCAAGTGGCAGCCAGGAGAAGAACCAG |
| | | | | CAGGCTCTGTGG TTCAGGACTCCCGACTTGACACGATATTTTTTGCTAAGCAG |
| 55813 | UTP6 | HSX2_02559089 | 903 | GAGAATCCTGATTATTCTGAAGAAATCCTTAAGGGCGAGTTGGCATGGATCAT |
| | | | | CTACAAAAATTC TGTAAGCATAATTAAAG |
| 4013 | VWA5A | HSX2_01038194 | 904 | GATAAAATGCCAATCAGGTTTTCGAAAGGCCTTA |
| 4013 | VWA5A | HSX2_02507460 | 905 | GATAAAATGCCAATCAGCACCATTCACCGCCTTG |
| 55339 | WDR33 | HSX2_01849878 | 906 | GGCCAGGTCATGAACATTTTCGTGATACTCCCCGCCCTGATCATCCCCCTCACG |
| | | | | ACGGTCATTCCC |
| | | | | CAGCCAGCAGAGAACGCTCCTCTTCTCTCCAAGGCATGGACATGGCATCCCTA |
| | | | | CCTCCCCGAAAG |
| | | | | CGCCCCTGGCATGATGGCCCAGGCACTTCTGAGCACAGAGAGATGGAGGCCC |
| | | | | CAGGAGGCCCTTCTGAAGACCGAGGAGGCAAAG |
| 9213 | XPR1 | HSX2_01392082 | 907 | GGAGGAAGATGGCGGGCGGGCTGCTCTGAAGAGACCTCGGCGGCGGCGGAG |
| | | | | GAGGAGAGAAG |
| | | | | CGCAGCGCCGCGCCGCGCCGGGGCCCATGTGGGGAGGAGTCGGAGTCGCTG |
| | | | | TTGCCGCCGCCG |
| | | | | CCTGTAGCTGCTGGACCCGAGTGGGAGTGAGGGGGAAACGGCAGGATGAAG |
| | | | | TTCGCCGAGCAC |
| | | | | CTCTCCGCGCACATCACTCCCGAGTGGAGGAAGCAATACATCCAGTATGAG |
| 9213 | XPR1 | HSX2_01392085 | 908 | GCTTTCAAGGATATGCTGTATTCAGCTCAGGACCAGGCACCTTCTGTGGAAG |
| 9213 | XPR1 | HSX2_01392094 | 909 | AGAAGCTCGCAGAGGCTCAGCGCAGGTTTGCTACACTTCAGAATGAGCTTCAG |
| | | | | TCATCACTGGAT |
| | | | | GCACAGAAAGAAAGCACTGGTGTTACTACGCTGCGACAACGCAGAAAGCCAG |
| | | | | TCTTCCACTTGTC |
| | | | | CCATGAGGAACGTGTCCAACATAGAAATATTAAAGACCTTAAACTGGCCTTCA |
| | | | | GTGAGTTCTACCT CAGTCTAATCCTGCTGCAGAACTATCAG |
| 9213 | XPR1 | HSX2_01392124 | 910 | TTTCGAGTATTTACAGCCCCCTTCCATAAGGTAGGCTTTGCTGATTTCTGGCTG |
| | | | | GCGGATCAGCTG |
| | | | | AACAGCCTGTCAGTGATACTGATGGACCTGGAATATATGATCTGCTTCTACAGT |
| | | | | TTGGAGCTCAA |
| | | | | ATGGGATGAAAGTAAGGGCCTGTTGCCAAATAATTCAGAAG |
| 9213 | XPR1 | HSX2_01392129 | 911 | AATCAGGAATTTGCCACAAATATACATATGGTGTGCGGGCCATTGTTCAGTGC |
| | | | | ATTCCTGCTTGGC |
| | | | | TTCGCTTCATCCAGTGCCTGCGCCGATATCGAGACACAAAAAGGGCCTTTCCTC |
| | | | | ATTTAGTTAATG |
| | | | | CTGGCAAATACTCCACAACTTTCTTCATGGTGACGTTTGCAGCCCTTTACAGCA |
| | | | | CTCACAAAG |
| XPR1 | 9213 | HSX2_01392144 | 912 | GCGATTTGTGTGGAACTTCTTCCGCCTGGAGAATGAACATCTGAATAACTGTG |
| | | | | GTGAATTCCGTG |
| | | | | CTGTGCGGGACATCTCTGTGGCCCCCCTGAACGCAGATGATCAGACTCTCCTA |
| | | | | GAACAGATGATG |
| | | | | GACCAGGATGATGGGGTACGAAACCGCCAGAAGAATCGGTCATGGAAGTACA |
| | | | | ACCAGAGCATAT CCCTGCGCCGGCCTCGCCTCGCTTCTCA |
| 65986 | ZBTB10 | HSX2_01994861 | 913 | GAGTTGATGCAGGACGGCGCGTCCCTGAGCGACAGCACCGAGGACGAGGAG |
| | | | | GAGGGGGCGAG |
| | | | | CCTGGGCGACGGCAGCGGGGCGGAAGGCGGCAGCTGCAGCAGCAGCAGGC |
| | | | | GGTCGGGCGGCG |
| | | | | ATGGCGGGGACGAAGTGGAGGGCAGCGGTGTGGGAGCTGGCGAAGGAGAG |
| | | | | ACTGTCCAGCAC |
| | | | | TTCCCGCTCGCGCGGCCCAAGTCTCTAATGCAGAAGCTCCAATGCTCCTTCCAG |
| | | | | ACCTCCTGGCTC |
| | | | | AAGGACTTTCCCTGGCTGCGCTATTCCAAGGATACTGGTCTTATGTCTTGCGGC |
| | | | | TGGTGCCAAAA |
| | | | | GACCCCTGCAGATGGGGGAAGCGTGGACCTTCCCCCAGTGGGGCATGATGAG |
| | | | | CTTTCGCGAGG |
| | | | | GACCCGCAACTACAAGAAAACCCTCCTCCTGAGGCACCACGTCTCTACCGAGC |
| | | | | ACAAACTCCACGAAGCCAACGCCCAG |
| 340595 | ZCCHC16 | HSX2_02359509 | 914 | CTTCACCTATTGACTACGATCTACCTTCAGCATAAATCAACCTTCCTGGAACCAC |
| | | | | AGCTCTGCCACC |
| | | | | CTTCCATAGAGCTATATGTTTTGTTTCCATCTAAAGACTCATGCTTTCCCAACAA |
| | | | | ACAGAACCTCCT |
| | | | | GGCTATTTACAAACCAGATTGAATGGGCATTTGAATACTGCTTGGAACTGTTG |
| | | | | GTCCCTTTTTCTTT |
| | | | | GCAGGCTAGCCCATCAGGCAGGTGTATGCTTTTAGCCAAGCCCACCAATTCCA |
| | | | | ACTTATTCCTCTC |
| | | | | TTCTCCAACAACCCTACTTGAGGTTCCAGCATGTCTTATAGCCAAGTCAATATG |
| | | | | CTCTAGGATAAG |
| | | | | GATTTCTGAAAGGTATTTAGTGAGAGTTGGGGATGGTGTGTCATAATTACTGT |
| | | | | ATCTGTAGCATG |
| | | | | TCCTTGCTGCCTTGGACAAGGGACCTGGCCAAATAAATCTCCACTGAAGACTG |
| | | | | ACAGCCAGACCC |
| | | | | TGCAGTAGTTTCTAACTCTAGAAGCCTCAATGTAGTCACCATCTAGGATTACTA |
| | | | | CTCATTGCTATTG |
| | | | | AGCTCTACCTTTATGGACCACCTCTGAAGTCCTTCATCTAATTGAGGATCTTAA |
| | | | | AAAACAATGAAT |
| | | | | GGCAAAAAACTTCCTACACTATCTCATCTGAAAAATCCTGGGGCACATACATTA |
| | | | | AAACCATTTGTG |
| | | | | CACAAAACTCAGGAAGTCGGGATTTTATTGCTTATGTTTTACTCTTGGTACACA |
| | | | | CCAGCTTTGTGT |
| | | | | AGATTCCTGATGCTTCTGAACAAAAGTAGTGTTATGCTAGAAAGGCAAAACTC |
| | | | | CCTAAAATGTTA |
| | | | | AAACTGTTCAGCAATGATCACTGCTGGTAATTACCAGCCCTCTGTTCCCTGCTT |
| | | | | CTTGTTTCTGATG |
| | | | | TATGTCCCCAAAATGGATATCCTGGAGAAATATCAGTAATCACATATTCCCTAG |
| | | | | CAACCATACCAT |
| | | | | TGGCCTTTTCCATGAAGAAGAGGCAACATTGAGTGGCGGACAACACTTTTTGG |
| | | | | CATCTCTCCAATT |
| | | | | CAGCTGTTAGCATCTCCTAGTGTCTTCTTCACAGCTAACAGCTTTGGCTACTTGC |
| | | | | TCATGACACTCC |
| | | | | GTCTCTGATCACAGAGCAAATCCACTGTCTCCATTGGTTTCCAGTACTAGTACT |
| | | | | CAATTCTACTCAA |
| | | | | TCCCACCTGATTCCAGGAGACATAATGGAGAAGTGCACGAAATCATCATCTAC |
| | | | | CATGCAGGTAGA |
| | | | | GCCTTCCTTTCTTCAGGCAGAGAATCTGATTCTGCGGCTTCAAATGCAGCATCC |
| | | | | AACCACGGAGA |
| | | | | ACACTGCTAAAAGGGGCCAAGTCATGCCTGCCCTGGCCACCACAGTGATGCCT |
| | | | | GTACCATACTCA |
| | | | | CTTGAGCATCTCACCCAGTTTCATGGTGACCCTGCCAATTGCTCAGAGTTCCTC |
| | | | | ACTCAGGTGACT |
| | | | | ACCTACTTGACAGCTCTCCAAATCTCTAATCCTGCAAATGATGCCCAGATCAAA |
| | | | | CTCTTTTTTGATT |
| | | | | ACCTATCTCAGCAGTTAGAAAGTTGTGGGATCATATCTGGGCCTGACAAGAGT |
| | | | | ACCTTACTGAAG |
| | | | | CAATATGAGAATCTTATTCTTGAGTTCCAGCAGTCATTTGGTAAACCCACAAAA |
| | | | | CAGGAAATCAAT |
| | | | | CCTCTGATGAATGCTAAGTTTGACAAAGGAGACAACTCCTCTCAGCAGGACCC |
| | | | | TGCTACTTTCCAC |
| | | | | CTCCTCGCTCAAAATCTGATCTGTAATGAAACCAATCAGAGTGGTCAGTTCGAA |
| | | | | AAGGCACTAGC |
| | | | | TGATCCCAACCAGGATGAAGAGAGTGTCACTGATATGATGGACAATCTTCCAG |
| | | | | ACCTGATCACTC |
| | | | | AGTGCATTCAGTTGGACAAGAAACATAGTGACAGGCCAGAGCTCCTACAGTCA |
| | | | | GAGACCCAGCT |
| | | | | CCCATTGTTGGCTTCCTTGATCCAACACCAAGCCCTCTTTAGCCCCACAGATCCA |
| | | | | CCACCCAAGAA |
| | | | | AGGGCCTATACAGCTGCGAGAAGGCCAGCTGCCTCTCACCCCAGCCAAACGA |
| | | | | GCCCGCCAGCAA |
| | | | | GAAACTCAGTTGTGCCTCTACTGCAGCCAATCTGGTCACTTCACAAGAGATTGC |
| | | | | CTTGCCAAACGT |
| | | | | TCTCGAGCTCCGGCAACGACAAATAACACAGCTCACCAGTAAGAGGAGACCA |
| | | | | GGAAAGTCACTT |
| | | | | TTTAAACTTACATCCCAGCCTTACTGATTTATAACCTGCATTAACTTTTAAAATA |
| | | | | CAGATGGGGAAC |
| | | | | TGTGACACCACTTTATAGGCAGTTATGAACTGACACGCTTTGGGGGAATTTAA |
| | | | | ACAATCAGATCTT |
| 51364 | ZMYND10 | HSX2_01761846 | 915 | GAGGTGTGTGAGTCAGCAGAAGACACTGTCTTGGACTTGGTAGACTATTGCCA |
| | | | | CCGCAAACTGA |
| | | | | CCCTGCTGGTGGCCCAGAGTGGCTGTGGTGGCCCCCCTGAGGGGGAGGGATC |
| | | | | CCAGGACAGCA ACCCCATGCAG |
| 155054 | ZNF425 | HSX2_02256272 | 916 | GTAACTGTGACATTTGATGATGTGGCCTTATATTTTTCGGAACAAGAGTGGGA |
| | | | | GATCCTGGAGAA |
| | | | | GTGGCAGAAGCAAATGTATAAGCAAGAGATGAAGACCAATTACGAGACCCTT |
| | | | | GATTCCCTGG |
| 57474 | ZNF490 | HSX2_01923238 | 917 | GACTCCATCTCCCTTGAGGATGTGGCTGTGAACTTCACCCTGGAGGAGTGGGC |
| | | | | TTTGCTGGATCC |
| | | | | TGGCCAGAGGAATATCTACAGAGATGTGATGCGGGCAACCTTCAAGAACCTG |
| | | | | GCCTGTATAG |
| 23140 | ZZEF1 | HSX2_01600756 | 918 | GTGCAGCAGAAGGAAAGTGGCTTAAAATATAAATCTTTTCTGGACTTCGCGGG |
| | | | | TCTTGATTTGCA GATCTTCTGGAATTTTTACAGTAAATTAAAGCAAAA |

1-27. (canceled)

28. A method for the diagnosis of thyroid cancer in a mammal, comprising measuring an expression product of at least the CITED1 and TENM1 genes in a sample from the mammal and diagnosing said thyroid cancer by comparing the measured value to a reference value.

29. The method of claim 28, for determining whether said mammal has or is likely to have a malignant thyroid lesion or nodule.

30. The method of claim 28, said method further comprising measuring an expression product of at least one further gene selected from CCM2, SCLY and IGSF10.

31. The method of claim 28, said method comprising measuring an expression product of at least the CITED1, TENM1, CCM2, SCLY and IGSF10 genes.

32. The method of claim 28, wherein measuring an expression product comprises determining the presence, absence or amount of said expression product.

33. The method of claim 28, said method comprising determining the presence, absence or amount of at least two distinct domains of an expression product of a CITED1 gene.

34. The method of claim 28, said method comprising determining the presence, absence or amount of a sequence encoded by exon4 of the CITED1 gene.

35. The method of claim 28, said method comprising determining the presence, absence or amount of at least one sequence encoded by exon4, exon3 and/or exon (−2) of the CITED1 gene.

36. The method of claim 28, said method comprising determining the presence, absence or amount of at least two distinct domains of an expression product of the TENM1 gene.

37. The method of claim 28, said method comprising determining the presence, absence or amount of at least one sequence encoded by exon1, 3, 13, 15, 17, 18, 19, 25, or 32 of the TENM1 gene.

38. The method of claim 28, said method comprising measuring an expression product of the FRMD3 gene.

39. The method of claim 28, said method comprising measuring an expression product of at least one gene listed in Table A.

40. The method of claim 28, said method comprising measuring an expression product of at least one gene listed in Table B.

41. The method of claim 28, said method comprising:
a) providing a biological sample from the subject having a thyroid condition;
b) performing a cytological exam of the sample to classify the thyroid condition in a category according to the Bethesda classification; and
c) for any sample of indeterminate cytology, diagnosing thyroid cancer by measuring in said biological sample an expression product.

42. The method of claim 28, wherein the gene products in the sample are measured simultaneously or sequentially.

43. The method of claim 28, wherein each gene product is measured by selective hybridization with specific nucleic acid probes and/or by selective amplification with specific nucleic acid primers.

44. The method of claim 28, wherein the measured value is compared to a reference value of a control benign thyroid condition and to a reference value of a control malignant thyroid condition, wherein, when the measured value is closer to the reference value of the control benign thyroid condition, then the tested thyroid condition is benign, and when the measured value is closer to the reference value of the control malignant thyroid condition, then the tested thyroid condition is malignant.

45. A method for determining in vitro or ex vivo the benign or malignant state of a thyroid condition in a subject, wherein said method comprises determining in a biological sample from the subject variation of expression of at least CITED1 and TENM1 genes.

46. The method of claim 45, wherein increased expression as compared to a control non-malignant thyroid tissue is indicative of a malignant status.

47. A product comprising a support on which are immobilised at least two distinct nucleic acid probes, each probe comprising a sequence complementary to and specific of a target nucleic acid selected from any one of the sequences listed as SEQ ID NOs: 1-918, or the complementary strand thereof.

48. The product of claim 47, comprising at least two probes selected from any one of the sequences listed as SEQ ID NOs: 919 to 968.
